(19) United States
(12) Patent Application Publication
(10) Pub. No.: US 2025/0262302 A1
(43) Pub. Date: Aug. 21, 2025

(54) Compositions and Methods for Reducing MHC Class I in a Cell

(71) Applicant: Intellia Therapeutics, Inc., Cambridge, MA (US)

(72) Inventors: Birgit Schultes, Arlington, MA (US); Yong Zhang, Bedford, MA (US); Boning Zhang, Cambridge, MA (US); Özgün Kiliç, Cambridge, MA (US); Ishina Balwani, Cambridge, MA (US); Biao Liu, Cambridge, MA (US)

(73) Assignee: Intellia Therapeutics, Inc., Cambridge, MA (US)

(21) Appl. No.: 18/980,462

(22) Filed: Dec. 13, 2024

Related U.S. Application Data
(63) Continuation of application No. PCT/US23/68498, filed on Jun. 15, 2023.
(60) Provisional application No. 63/494,208, filed on Apr. 4, 2023.
(60) Provisional application No. 63/352,991, filed on Jun. 16, 2022.

(51) Int. Cl. A61K 40/22; A61K 40/31; C12N 9/22; C12N 15/11; C12N 15/90

(52) U.S. Cl. CPC A61K 40/22; A61K 40/31; C12N 9/226; C12N 15/11; C12N 15/907; C12N 2310/20

(57) ABSTRACT

Compositions and methods for reducing MHC class I protein expression in a cell comprising genetically modifying MHC class I for use e.g., in adoptive cell transfer therapies.

### I. CROSS-REFERENCE TO RELATED APPLICATION

This application is a continuation of International Application No. PCT/US2023/068498, filed on Jun. 15, 2023, which claims the benefit under 35 USC 119(e) of U.S. Provisional Application No. 63/352,991, filed Jun. 16, 2022, and U.S. Provisional Application No. 63/494,208, filed Apr. 4, 2023, the content of each of which is herein incorporated by reference in its entirety.

### II. REFERENCE TO AN ELECTRONIC SEQUENCE LISTING

This application contains a sequence listing, which has been submitted electronically in XML file format and is hereby incorporated by reference in its entirety. Said XML file, created on Jun. 13, 2023, is named “01155-0054-OOPCT_SL.xml” and is 14,122,813 bytes in size.

### III. INTRODUCTION AND SUMMARY

The ability to downregulate MHC class I is critical for many in vivo and ex vivo utilities, e.g., when using allogeneic cells (originating from a donor) for transplantation or e.g., for creating a cell population in vitro that does not activate T cells. In particular, the transfer of allogeneic cells into a subject is of great interest to the field of cell therapy. The use of allogeneic cells has been limited due to the problem of rejection by the recipient subject's immune cells, which recognize the transplanted cells as foreign and mount an attack. To avoid the problem of immune rejection, cell-based therapies have focused on autologous approaches that use a subject's own cells as the cell source for therapy, an approach that is time-consuming and costly.

Typically, immune rejection of allogeneic cells results from a mismatching of major histocompatibility complex (MHC) molecules between the donor and recipient. Within the human population, MHC molecules exist in various forms, including e.g., numerous genetic variants of any given MHC gene, i.e., alleles, encoding different forms of MHC protein. The primary classes of MHC molecules are referred to as MHC class I and MHC class II. MHC class I molecules (e.g., HLA-A, HLA-B, and HLA-C in humans) are expressed on all nucleated cells and present antigens to activate cytotoxic T cells (CD8+ T cells or CTLs). MHC class II molecules (e.g., HLA-DP, HLA-DQ, and HLA-DR in humans) are expressed on only certain cell types (e.g., B cells, dendritic cells, and macrophages) and present antigens to activate helper T cells (CD4+ T cells or Th cells), which in turn provide signals to B cells to produce antibodies.

Slight differences, e.g., mismatches in MHC alleles between individuals can cause the T cells in a recipient to become activated. During T cell development, an individual's T cell repertoire is tolerized to one's own MHC molecules, but T cells that recognize another individual's MHC molecules may persist in circulation and are referred to as alloreactive T cells. Alloreactive T cells can become activated e.g., by the presence of another individual's cells expressing MHC molecules in the body, causing e.g., graft versus host disease and transplant rejection.

While fully matching HLA types between donor and recipient is theoretically possible as a means of reducing transplant rejection, such an approach is logistically and practically challenging given the diversity of HLA alleles across the population to fully match e.g., 10 out 10 alleles (i.e., 2 alleles for each of HLA-A, HLA-B, HLA-C, HLA-DRB1, and HLA-DQB1).

Methods and compositions for reducing the susceptibility of an allogeneic cell to rejection are of interest, including e.g., reducing the cell's expression of MHC protein to avoid recipient T cell responses. In practice, the ability to genetically modify an allogeneic cell for transplantation into a subject has been hampered by the requirement for multiple gene edits to reduce all MHC protein expression, while at the same time, avoiding other harmful recipient immune responses. For example, while strategies to deplete MHC class I protein may reduce activation of CTLs, cells that lack MHC class I on their surface are susceptible to lysis by natural killer (NK) cells of the immune system because NK cell activation is regulated by MHC class I-specific inhibitory receptors. Moreover, while several groups have studied the NK protective effects of the different MHC class I components, some inconsistencies remain in the field. See, e.g., Keystone 2022 presentation “Multiplex Base Editing Mitigates CAR-T Cell Allorejection” by Beam Therapeutics—Workshop: Therapeutic Applications session, Apr. 29, 2022 (“Keystone Presentation”). For example, the Keystone Presentation concluded that retention of HLA-A, not HLA-B or —C, afforded protection against NK killing in vitro, while other groups have shown that HLA-C is important in inhibiting NK activity (Xu et al. 2019, Cell Stem Cell 24, 566-578). Further still, earlier studies suggest HLA-E and HLA-G provide some but not complete protection. Therefore, safely reducing or eliminating expression of MHC class I has proven challenging.

Gene editing strategies to deplete MHC class II molecules have also proven difficult particularly in certain cell types for reasons including low editing efficiencies and low cell survival rates, preventing practical application as a cell therapy.

Thus, there exists a need for improved methods and compositions for modifying allogeneic cells to overcome the problem of recipient immune rejection and the technical difficulties associated with the multiple genetic modifications required to produce a safer cell for transplant.

The present disclosure provides engineered human cells with reduced or eliminated surface expression of HLA-B protein relative to an unmodified cell, wherein the cell is homozygous for HLA-A and HLA-C, or wherein the cell has reduced or eliminated surface expression of HLA-A and HLA-B protein relative to an unmodified cell and is homozygous for HLA-C. The engineered human cells disclosed herein therefore provide a “partial matching” approach to the problem of allogeneic cell transfer and MHC class I compatibility. The use of cells that have reduced or eliminated expression of HLA-B and are homozygous for HLA-A and HLA-C, or that have reduced or eliminated expression of HLA-A and HLA-B and are homozygous for HLA-C, limits the number of donors that are necessary to provide a therapy that covers a majority of recipients in population because the disclosed partial matching approach requires only one matching HLA-A allele (as opposed to two) and only one HLA-C allele (as opposed to two), or requires only one matching HLA-C allele (as opposed to two). Surprisingly, the engineered human cells that have reduced or eliminated surface expression of HLA-B protein only or both HLA-A and HLA-B protein relative to an unmodified cell, disclosed herein, demonstrate persistence and are protective against NK-mediated rejection, especially as compared to engineered cells with reduced or eliminated B2M expression. The disclosure provides methods and compositions for generating such engineered human cells with reduced or eliminated surface expression of HLA-B protein only or both HLA-A and HLA-B protein relative to an unmodified cell, wherein the cell is homozygous for both HLA-A and HLA-C or HLA-C only. In some embodiments, the disclosure provides engineered human cells, and methods and compositions for generating engineered human cells, wherein the cell further has reduced or eliminated expression of MHC class II protein on the surface of the cell, e.g., wherein the cell has a genetic modification in the CIITA gene. In some embodiments, the disclosure provides for further engineering of the cell, including to reduce or eliminate the expression of endogenous T cell receptor proteins (e.g., TRAC, TRBC), and to introduce an exogenous nucleic acid, e.g., encoding a polypeptide expressed on the cell surface or a polypeptide that is secreted by the cell. Thus, the disclosure thus provides a flexible platform for genetically engineering human cells for a variety of desired adoptive cell therapy purposes.

Provided herein is an engineered human cell, which has reduced or eliminated surface expression of HLA-B protein relative to an unmodified cell, comprising a genetic modification in the HLA-B gene, wherein the cell is homozygous for HLA-A and homozygous for HLA-C. Also provided is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: (a) chr6:31354480-31357174 or (b) chr6:31354623-31357108 or 31354497-31357157, wherein the cell is homozygous for HLA-A and homozygous for HLA-C.

Provided herein is an engineered human cell, which has reduced or eliminated surface expression of HLA-A and HLA-B protein relative to an unmodified cell, comprising (i) a genetic modification in the HLA-A gene, wherein the genetic modification in the HLA-A gene comprises at least one nucleotide within the genomic coordinates chosen from chr6: 29942854-chr6:29942913 and chr6:29943518-chr6:29943619, and (ii) a genetic modification in the HLA-B gene, wherein the genetic modification in the HLA-B gene comprises at least one nucleotide within the genomic coordinates chosen from: (a) chr6:31354480-31357174 or (b) chr6: 31354623-31357108 or 31354497-31357157, wherein the cell is homozygous for HLA-C.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355182-31355202; chr6:31355348-31355368; chr6:31355145-31355165; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355347-31355367; chr6:31355432-31355452; chr6:31355340-31355360; chr6:31355410-31355430; chr6:31355414-31355434; or chr6:31355409-31355429.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355182-31355202; chr6:31355349-31355369; chr6:31355348-31355368; or chr6:31355145-31355165.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355348-31355368; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; or chr6:31355414-31355434.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355348-31355368; chr6:31355349-31355369; chr6:31356381-31356401; chr6:31356380-31356400; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; chr6:31355410-31355430; chr6:31355414-31355434; or chr6:31355409-31355429.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355348-31355368, chr6:31355349-31355369, chr6:31355192-31355212, chr6:31355347-31355367, chr6:31355340-31355360, chr6:31355409-31355429.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355349-31355369 or chr6:31355348-31355368.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355192-31355212 or chr6:31355347-31355367.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355347-31355367; chr6:31355340-31355360; or chr6:31355409-31355429.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355348-31355368; chr6:31355145-31355165; chr6:31355347-31355367; chr6:31355432-31355452; or chr6:31355340-31355360.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; chr6:31355469-31355493; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355361-31355385; chr6:31355379-31355403; chr6:31355491-31355515; chr6:31355356-31355380; chr6:31355366-31355390; chr6:31355417-31355441; chr6:31357078-31357102; chr6:31355460-31355484; chr6:31355415-31355439; chr6:31355166-31355190; chr6:31355378-31355402; chr6:31355401-31355425; chr6:31356262-31356286; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; or chr6:31356426-31356450.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355379-31355403; chr6:31355491-31355515; chr6:31355361-31355385; chr6:31355221-31355245; chr6:31355222-31355246; chr6:31355205-31355229; chr6:31355446-31355470; chr6:31356425-31356449; or chr6:31355441-31355465.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355221-31355245; chr6:31355222-31355246; chr6:31355205-31355229; chr6:31355446-31355470; chr6:31356425-31356449; or chr6:31355441-31355465.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; chr6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

Provided herein is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: (a) chr6:31355348-31355368; or (b) chr6:31355390-31355414; chr6:31355417-31355441; or chr6: 31356386-31356410.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355182-31355202; chr6:31355348-31355368; chr6:31355145-31355165; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355347-31355367; chr6:31355432-31355452; chr6:31355340-31355360; chr6:31355410-31355430; chr6:31355414-31355434; or chr6:31355409-31355429.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355182-31355202; chr6:31355349-31355369; chr6:31355348-31355368; or chr6:31355145-31355165.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355348-31355368; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; or chr6:31355414-31355434.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355348-31355368; chr6:31355349-31355369; chr6:31356381-31356401; chr6:31356380-31356400; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; chr6:31355410-31355430; chr6:31355414-31355434; or chr6:31355409-31355429.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355348-31355368, chr6:31355349-31355369, chr6:31355192-31355212, chr6:31355347-31355367, chr6:31355340-31355360, chr6:31355409-31355429.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355349-31355369 or chr6:31355348-31355368.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355192-31355212 or chr6:31355347-31355367.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355347-31355367; chr6:31355340-31355360; or chr6:31355409-31355429.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355348-31355368; chr6:31355145-31355165; chr6:31355347-31355367; chr6:31355432-31355452; or chr6:31355340-31355360.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; chr6:31355469-31355493; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31356777-31356801; chr6:31355492-31355516; chr6:31355361-31355385; chr6:31355379-31355403; chr6:31355491-31355515; chr6:31355356-31355380; chr6:31355366-31355390; chr6:31355417-31355441; chr6:31357078-31357102; chr6:31355460-31355484; chr6:31355415-31355439; chr6:31355166-31355190; chr6:31355378-31355402; chr6:31355401-31355425; chr6:31356262-31356286; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; or chr6:31356426-31356450.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31356777-31356801; chr6:31355492-31355516; chr6:31355379-31355403; chr6:31355491-31355515; chr6:31355361-31355385.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; chr6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

Provided herein is an engineered human cell, which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: (a) chr6:31355348-31355368; or (b) chr6:31355390-31355414; chr6:31355417-31355441; or chr6: 31356386-31356410.

Provided herein is a method of making an engineered human cell, which has reduced or eliminated surface expression of HLA-B protein relative to an unmodified cell, wherein the cell is homozygous for HLA-A and homozygous for HLA-C, comprising contacting a cell with a composition comprising: (a) an HLA-B guide RNA comprising (i) a guide sequence selected from SEQ ID NOs: 1-91 and 101-185; or (ii) at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or at least 95%, 90%, 85%, 80%, 75%, or 70% identical to a sequence selected from SEQ ID NOs: 101-185; or (iv) a guide sequence that binds a target site comprising a genomic region listed in Tables 2 or 3; or (v) a guide sequence that is complementary to at least 17, 18, 19, or 20 contiguous nucleotides of a genomic region listed in Table 2, or a guide sequence that is complementary to at least 17, 18, 19, 20, 21, 22, 23, or 24 contiguous nucleotides of a genomic region listed in Table 3; or (vi) a guide sequence that is at least 95%, 90%, or 85%, 80%, 75%, or 70% identical to a sequence selected from (v); and optionally (b) an RNA-guided DNA binding agent or a nucleic acid encoding an RNA-guided DNA binding agent.

Provided herein is a method of making an engineered human cell, which as reduced or eliminated surface expression of HLA-A and HLA-B protein relative to an unmodified cell, wherein the cell is homozygous for HLA-C, comprising contacting a cell with a first composition comprising (a) an HLA-A guide RNA comprising (i) a guide sequence selected from SEQ ID NOs: 301-590; or (ii) at least 17, 18, 19, or 20 contiguous nucleotides of a sequence selected from SEQ ID NOs: 301-428 and 463-511; or at least 17, 18, 19, 20, 21, 22, 23, or 24 contiguous nucleotides of a sequence selected from SEQ ID NOs 429-462 and 512-590; or (iii) a guide sequence at least 95%, 90%, or 85% identical to a sequence selected from SEQ ID NOs: 301-428 and 463-511; or at least 95%, 90%, 85%, 80%, 75%, or 70% identical to a sequence selected from SEQ ID NOs: 429-462 and 512-590; or (iv) a guide sequence that binds a target site comprising a genomic region listed in Tables 4-7; or (v) a guide sequence that is complementary to at least 17, 18, 19, or 20 contiguous nucleotides of a genomic region listed in Table 4, Table 5B or Table 6; or a guide sequence that is complementary to at least 17, 18, 19, 20, 21, 22, 23, or 24 contiguous nucleotides of a genomic region listed in Table 5A or Table 7; or (vi) a guide sequence that is at least 95%, 90%, 85%, 80%, 75%, or 70% identical to a sequence selected from (v); and optionally (b) a first RNA-guided DNA binding agent or a nucleic acid encoding an RNA-guided DNA binding agent; and contacting a cell with a second composition comprising (a) an HLA-B guide RNA comprising (i) a guide sequence selected from SEQ ID NOs: 1-91 and 101-185; or (ii) at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or at least 95%, 90%, 85%, 80%, 75%, or 70% identical to a sequence selected from SEQ ID NOs: 101-185; or (iv) a guide sequence that binds a target site comprising a genomic region listed in Table 2 or 3; or (v) a guide sequence that is complementary to at least 17, 18, 19, or 20 contiguous nucleotides of a genomic region listed in Table 2, or a guide sequence that is complementary to at least 17, 18, 19, 20, 21, 22, 23, or 24 contiguous nucleotides of a genomic region listed in Table 3; or (vi) a guide sequence that is at least 95%, 90%, 85%, 80%, 75%, or 70% identical to a sequence selected from (v); and optionally (b) an RNA-guided DNA binding agent or a nucleic acid encoding an RNA-guided DNA binding agent.

Provided herein is a method of reducing surface expression of HLA-B protein in a human cell relative to an unmodified cell, comprising contacting a cell with composition comprising: (a) a HLA-B guide RNA comprising (i) a guide sequence selected from SEQ ID NOs: 1-91 and 101-185; or (ii) at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or at least 95%, 90%, 85%, 80%, 75%, or 70% identical to a sequence selected from SEQ ID NOs: 101-185; or (iv) a guide sequence that binds a target site comprising a genomic region listed in Table 2 or 3; or (v) a guide sequence that is complementary to at least 17, 18, 19, or 20 contiguous nucleotides of a genomic region listed in Table 2, or a guide sequence that is complementary to at least 17, 18, 19, 20, 21, 22, 23, or 24 contiguous nucleotides of a genomic region listed in Table 3; or (vi) a guide sequence that is at least 95%, 90%, 85%, 80%, 75%, or 70% identical to a sequence selected from (v); and optionally (b) an RNA-guided DNA binding agent or a nucleic acid encoding an RNA-guided DNA binding agent.

Provided herein is a method of reducing surface expression of HLA-A and HLA-B protein in a human cell relative to an unmodified cell, comprising contacting a cell with composition comprising: contacting a cell with a first composition comprising (a) an HLA-A guide RNA comprising (i) a guide sequence selected from SEQ ID NOs: 301-590; or (ii) at least 17, 18, 19, or 20 contiguous nucleotides of a sequence selected from SEQ ID NOs: 301-428 and 463-511; or at least 17, 18, 19, 20, 21, 22, 23, or 24 contiguous nucleotides of a sequence selected from SEQ ID NOs: 429-462 and 512-590; or (iii) a guide sequence at least 95%, 90%, or 85% identical to a sequence selected from SEQ ID NOs: 301-428 and 463-511; or at least 95%, 90%, 85%, 80%, 75%, or 70% identical to a sequence selected from SEQ ID NOs: 429-462 and 512-590; or (iv) a guide sequence that binds a target site comprising a genomic region listed in Tables 4-7; or (v) a guide sequence that is complementary to at least 17, 18, 19, or 20 contiguous nucleotides of a genomic region listed in Table 4, 5B or 6; or a guide sequence that is complementary to at least 17, 18, 19, 20, 21, 22, 23, or 24 contiguous nucleotides of a genomic region listed in Table 5A or Table 7; or (vi) a guide sequence that is at least 95%, 90%, 85%, 80%, 75%, or 70% identical to a sequence selected from (v); and optionally (b) a first RNA-guided DNA binding agent or a nucleic acid encoding an RNA-guided DNA binding agent; and contacting a cell with a second composition comprising (a) an HLA-B guide RNA comprising (i) a guide sequence selected from SEQ ID NOs: 1-91 and 101-185; or (ii) at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or at least 95%, 90%, 85%, 80%, 75%, or 70% identical to a sequence selected from SEQ ID NOs: 101-185; or (iv) a guide sequence that binds a target site comprising a genomic region listed in Table 2 or 3; or (v) a guide sequence that is complementary to at least 17, 18, 19, or 20 contiguous nucleotides of a genomic region listed in Table 2, or a guide sequence that is complementary to at least 17, 18, 19, 20, 21, 22, 23, or 24 contiguous nucleotides of a genomic region listed in Table 3; or (vi) a guide sequence that is at least 95%, 90%, 85%, 80%, 75%, or 70% identical to a sequence selected from (v); and optionally (b) an RNA-guided DNA binding agent or a nucleic acid encoding an RNA-guided DNA binding agent.

Provided herein is a method of administering an engineered cell to a recipient subject in need thereof, the method comprising: (a) determining the HLA-A and HLA-C alleles of the recipient subject; (b) selecting an engineered cell or cell population of any one of the preceding embodiments, or engineered cell or cell population produced by the method of any one of the preceding embodiments, wherein the engineered cell comprises at least one of the same HLA-A or HLA-C alleles as the recipient subject; (c) administering the selected engineered cell to the recipient subject.

Provided herein is a method of administering an engineered cell to a recipient subject in need thereof, the method comprising (a) determining the HLA-C alleles of the recipient subject; (b) selecting an engineered cell or cell population of any one of the preceding embodiments, or engineered cell or cell population produced by the method of any one of the preceding embodiments, wherein the engineered cell comprises at least one of the same HLA-C alleles as the recipient subject; (c) administering the selected engineered cell to the recipient subject.

Further embodiments are provided throughout and described in the claims and Figures.

### IV. BRIEF DESCRIPTION OF THE DRAWINGS

FIG. 1 shows the mean percentage of cells negative for HLA-B7 following editing at the HLA-B locus using 100-mer Spy guides.

FIG. 2 shows the percentage of T cell lysis following NK cell challenge after editing with various Spy sgRNAs.

FIGS. 3A-3E show the mean percentage of cells negative for HLA-B following editing at the HLA-B locus. FIG. 3A-3C show the mean percentage of HLA-B-cells across three donors in 100-mer Spy guides and four 91-mer Spy guides. FIG. 3D-3E show the mean percentage of HLA-B-cells across two donors in 91-mer Spy guides following editing at the HLA-B locus.

FIGS. 4A-B show the mean percentage of HLA-B knockout. FIG. 4A shows the mean percentage of HLA-B*07:02 knockout and FIG. 4B shows the mean percentage of HLA*B08:01 knockout.

FIGS. 5A-C shows the mean percentage of cells negative for HLA-B7 following editing at the HLA-B locus with various Nine sgRNAs. FIG. 5A shows HLA-B7 negative cells in cells with Nme2 BC22n guides. FIGS. 5B-C show HLA-B7 negative cells in cells treated with Nme2 Cleavase guides. FIG. 5B shows the mean percentage of HLA-B*07:02 knockout and FIG. 5C shows the mean percentage of HLA-B*08:01 knockout.

FIG. 6 shows the dose response curve for the percent of HLA-A2− of CD8+ cells with various doses of Nine sgRNA following editing at the HLA-B locus.

FIG. 7 shows the dose response curve for the percent of HLA-B7− of CD8+ cells with various doses of Nine sgRNA following editing at the HLA-B locus.

FIG. 8A shows the mean percentage of cells negative for HLA-B7 following editing using candidate guides at the HLA-B locus with an Nme2 base editor (deaminase, also referred to as BC22n). G028907 was used as a control.

FIG. 8B shows the mean percentage of cells negative for and HLA-B8 following editing using candidate guides at the HLA-B locus with an Nme2 base editor (deaminase, also referred to as BC22n). G028907 was used as a control.

FIG. 9 shows the percentage of T cell lysis following NK cell challenge to engineered T cells with HLA-A, HLA-B, or HLA-A/B knockout.

FIGS. 10A-10D show the percent editing at each sgRNA dose in either HLA-B homozygous or heterozygous donors. FIG. 10A shows percent of HLA-B7− and CD8+ cells in an HLA-B7 homozygous donor. FIG. 10B shows percent of HLA-B8− and CD8+ cells in an HLA-B7 homozygous donor. FIG. 10C shows percent of HLA-B7− and CD8+ cells in an HLA-B7 heterozygous donor. FIG. 10D shows percent of HLA-B8− and CD8+ cells in an HLA-B7 heterozygous donor.

FIG. 11 shows the total flux (photons/s) from luciferase expressing T cells present at the various time points after injection for cells edited with HLA-A, HLA-B, CIITA, TRAC, and/or B2M.

FIG. 12 shows the total flux (photons/s) from luciferase expressing T cells present at the various time points after injection for cells edited with HLA-A, HLA-B, CIITA, TRAC, and/or B2M.

FIGS. 13A and 13B show the percentage killing results in tumor cells. FIG. 13A shows the percentage killing results in HH cells for double and triple KO edits. FIG. 13B shows the percentage killing results in MOLT-4 cells for double and triple KO edits.

FIG. 14 shows the % T cell killing results with NK cells for T cells with different edits or controls of B2M/CIITA KO, unedited, or untransduced T cells.

FIGS. 15A and 15B show the percentage of host T cell proliferation when co-cultured with engineered donor T cells.

### V. DETAILED DESCRIPTION

The present disclosure provides engineered human cells, as well as methods and compositions for genetically modifying a human cell to make engineered human cells that are useful, for example, for adoptive cell transfer (ACT) therapies. The disclosure provides engineered human cells with reduced or eliminated surface expression of HLA-B protein relative to an unmodified cell, wherein the cell is homozygous for HLA-A and homozygous for HLA-C. Additionally, the disclosure provides engineered human cells with reduced or eliminated surface expression of HLA-A and HLA-B protein relative to an unmodified cell, wherein the cell is homozygous for HLA-C. Thus, the engineered human cells disclosed herein provide a “partial matching” solution to hurdles associated with allogeneic cell transfer.

In some embodiments, the disclosure provides engineered human cells with reduced or eliminated surface expression of HLA-B protein as a result of a genetic modification in the HLA-B gene, wherein the cell is homozygous for HLA-A and HLA-C. In some embodiments, the disclosure provides compositions and methods for reducing or eliminating expression of HLA-B protein relative to an unmodified cell and compositions and methods to reduce the cell's susceptibility to immune rejection. In some embodiments, the engineered human cells with reduced or eliminated surface expression of HLA-B protein relative to an unmodified cell are not susceptible to lysis by NK cells, a problem observed with other approaches that reduce or eliminate MHC class I protein expression. In some embodiments, the methods and compositions comprise reducing or eliminating surface expression of HLA-B protein by genetically modifying HLA-B with a gene editing system, and inserting an exogenous nucleic acid encoding a targeting receptor, or other polypeptide (expressed on the cell surface or secreted) into the cell by genetic modification. The engineered cell compositions produced by the methods disclosed herein have desirable properties, including e.g., reduced or eliminated expression of HLA-B, reduced immunogenicity in vitro and in vivo, increased survival, and increased genetic compatibility with greater subjects for transplant.

In some embodiments, the disclosure provides engineered human cells with reduced or eliminated surface expression of HLA-A and HLA-B protein as a result of a genetic modification in the HLA-A and HLA-B genes, wherein the cell is homozygous for HLA-C. In some embodiments, the disclosure provides compositions and methods for reducing or eliminating expression of HLA-A and HLA-B protein relative to an unmodified cell and compositions and methods to reduce the cell's susceptibility to immune rejection. In some embodiments, the engineered human cells with reduced or eliminated surface expression of HLA-A and HLA-B protein relative to an unmodified cell are not susceptible to lysis by NK cells, a problem observed with other approaches that reduce or eliminate MHC class I protein expression. In some embodiments, the methods and compositions comprise reducing or eliminating surface expression of HLA-A and HLA-B protein by genetically modifying HLA-A and HLA-B with a gene editing system, and inserting an exogenous nucleic acid encoding a targeting receptor, or other polypeptide (expressed on the cell surface or secreted) into the cell by genetic modification. The engineered cell compositions produced by the methods disclosed herein have desirable properties, including e.g., reduced or eliminated surface expression of HLA-A and HLA-B protein, reduced immunogenicity in vitro and in vivo, increased survival, and increased genetic compatibility with greater subjects for transplant.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Provided herein are the following numbered embodiments:

Embodiment 1 is an engineered human cell, which has reduced or eliminated surface expression of HLA-A and HLA-B protein relative to an unmodified cell, comprising a genetic modification in the HLA-A gene and a genetic modification in the HLA-B gene, wherein the cell is homozygous for HLA-C.

Embodiment 2 is an engineered human cell, which has reduced or eliminated surface expression of HLA-A and HLA-B protein relative to an unmodified cell, comprising a genetic modification in the HLA-A gene and a genetic modification in the HLA-B gene, wherein (i) the genetic modification in the HLA-A gene comprises at least one nucleotide within the genomic coordinates chosen from: (a) chr6:29942854-chr6:29942913 and chr6:29943518-chr6:29943619; and (b) chr6:29942540-29945459; (ii) the genetic modification in the HLA-B gene comprises at least one nucleotide within the genomic coordinates chosen from: (a) chr6:31354480-31357174 or (b) chr6: 31354497-31357157; wherein the cell is homozygous for HLA-C.

Embodiment 3 is an engineered human cell, which has reduced or eliminated surface expression of HLA-B protein relative to an unmodified cell, comprising a genetic modification in the HLA-B gene, wherein the cell is homozygous for HLA-A and homozygous for HLA-C.

Embodiment 4 is an engineered human cell, which has reduced or eliminated surface expression of HLA-B protein relative to an unmodified cell, comprising a genetic modification in the HLA-B gene, wherein the genetic modification comprises at least one nucleotide within the genomic coordinates chosen from: (a) chr6:31354480-31357174 or (b) chr6: 31354497-31357157; wherein the cell is homozygous for HLA-A and homozygous for HLA-C.

Embodiment 5 is the engineered human cell of any one of embodiments 1-4, wherein the cell has reduced or eliminated expression of at least one HLA-B allele selected from HLA-B7, HLA-B8, HLA-B35, HLA-B40, HLA-B44, HLA-B15, HLA-B14, HLA-B18 and HLA-B51.

Embodiment 6 is the engineered human cell of any one of embodiments 1, 2, or 5, wherein the cell has reduced or eliminated expression of at least one HLA-A allele selected from: HLA-A1, HLA-A2, HLA-A3, HLA-AT1, HLA-A29, HLA-A26, HLA-A33, and HLA-A24.

Embodiment 7 is the engineered cell of any one of embodiments 1-6, wherein the genetic modification in the HLA-B gene comprises at least one nucleotide within the genomic coordinates chosen from: (a) chr6:31355182-31355596 or (b) chr6: 31355203-31356461.

Embodiment 8 is the engineered cell of any one of embodiments 1-7, wherein the genetic modification in the HLA-B comprises at least one nucleotide within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; or chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

Embodiment 9 is the engineered cell of any of embodiments 1-8, wherein the genetic modification in the HLA-B comprises at least one nucleotide within the genomic coordinates chosen from: chr6:31355182-31355202; chr6:31355348-31355368; chr6:31355145-31355165; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355347-31355367; chr6:31355432-31355452; chr6:31355340-31355360; chr6:31355410-31355430; chr6:31355414-31355434; or chr6:31355409-31355429.

Embodiment 10 is the engineered cell of any of embodiments 1-9, wherein the genetic modification in the HLA-B comprises at least one nucleotide within the genomic coordinates chosen from: chr6:31355182-31355202; chr6:31355349-31355369; chr6:31355348-31355368; or chr6:31355145-31355165.

Embodiment 11 is the engineered cell of any of embodiments 1-10, wherein the genetic modification in the HLA-B comprises at least one nucleotide within the genomic coordinates chosen from: chr6:31355348-31355368; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; or chr6:31355414-31355434.

Embodiment 12 is the engineered cell of any of embodiments 1-11, wherein the genetic modification in the HLA-B comprises at least one nucleotide within the genomic coordinates chosen from: chr6:31355348-31355368; chr6:31355349-31355369; chr6:31356381-31356401; chr6:31356380-31356400; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; chr6:31355410-31355430; chr6:31355414-31355434; or chr6:31355409-31355429.

Embodiment 13 is the engineered cell of any of embodiments 1-12, wherein the genetic modification in the HLA-B comprises at least one nucleotide within the genomic coordinates chosen from: chr6:31355348-31355368, chr6:31355347-31355367, chr6:31355349-31355369, chr6:31355192-31355212, chr6:31355340-31355360, chr6:31355409-31355429.

Embodiment 14 is the engineered cell of any of embodiments 1-13, wherein the genetic modification in the HLA-B comprises at least one nucleotide within the genomic coordinates chosen from: (i) chr6:31355349-31355369 or chr6:31355348-31355368; (ii) chr6:31355192-31355212 or chr6:31355347-31355367; (iii) chr6:31355347-31355367; chr6:31355340-31355360; or chr6:31355409-31355429; or (iv) chr6:31355348-31355368; chr6:31355145-31355165; chr6:31355347-31355367; chr6:31355432-31355452; or chr6:31355340-31355360.

Embodiment 15 is the engineered cell of any of embodiments 1-14, wherein the genetic modification in the HLA-B comprises at least one nucleotide within the genomic coordinates chosen from: chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

Embodiment 16 is the engineered cell of any of embodiments 1-15, wherein the genetic modification in the HLA-B comprises at least one nucleotide within the genomic coordinates chosen from: chr6:31356777-31356801; chr6:31355492-31355516; chr6:31355361-31355385; chr6:31355379-31355403; chr6:31355491-31355515; chr6:31355356-31355380; chr6:31355366-31355390; chr6:31355417-31355441; chr6:31357078-31357102; chr6:31355460-31355484; chr6:31355415-31355439; chr6:31355166-31355190; chr6:31355378-31355402; chr6:31355401-31355425; chr6:31356262-31356286; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

Embodiment 17 is the engineered cell of any of embodiments 1-16, wherein the genetic modification in the HLA-B comprises at least one nucleotide within the genomic coordinates chosen from: chr6:31356777-31356801; chr6:31355492-31355516; chr6:31355379-31355403; ch6:31355491-31355515; chr6:31355361-31355385; chr6:31355356-31355380; chr6:31355460-31355484; chr6:31357078-31357102; chr6:31355417-31355441; chr6:31355366-31355390; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; or chr6:31356426-31356450.

Embodiment 18 is the engineered cell of any of embodiments 1-17, wherein the genetic modification in the HLA-B comprises at least one nucleotide within the genomic coordinates chosen from: chr6:31356777-31356801; chr6:31355492-31355516; chr6:31355379-31355403; ch6:31355491-31355515; chr6:31355361-31355385; chr6:31355221-31355245; chr6:31355222-31355246; chr6:31355205-31355229; chr6:31355446-31355470; chr6:31356425-31356449; or chr6:31355441-31355465.

Embodiment 19 is the engineered cell of any of embodiments 1-18, wherein the genetic modification in the HLA-B comprises at least one nucleotide within the genomic coordinates chosen from: chr6:31355221-31355245; chr6:31355222-31355246; chr6:31355205-31355229; chr6:31355446-31355470; chr6:31356425-31356449; or chr6:31355441-31355465.

Embodiment 20 is the engineered cell of any one of embodiments 1-19, wherein the genetic modification in the HLA-B comprises at least one nucleotide within the genomic coordinates chosen from: (a) chr6:31355348-31355368; or (b) chr6:31355390-31355414; chr6:31355417-31355441; or chr6: 31356386-31356410.

Embodiment 21 is the engineered cell of any one of embodiments 1-2 and 5-20, wherein the genetic modification in HLA-A comprises at least one nucleotide within the genomic coordinates chosen from: chr6:29942864-29942884; chr6:29942868-29942888; chr6:29942876-29942896; chr6:29942877-29942897; and chr6:29942883-29942903.

Embodiment 22 is the engineered cell of any one of embodiments 1-2 and 5-21, wherein the genetic modification in HLA-A comprises at least one nucleotide within the genomic coordinates chosen from: chr6:29942891-29942915; chr6:29942609-29942633; chr6:29942864-29942884; chr6:29944266-29944290; chr6:29942889-29942913; chr6:29942891-29942915chr6:29944471-29944495; chr6:29944470-29944494.

Embodiment 23 is the engineered cell of any one of embodiments 1-2 and 5-22, wherein the genetic modification in HLA-A comprises at least one nucleotide within the genomic coordinates chosen from: chr6:29942891-29942915; chr6:29942609-29942633.

Embodiment 24 is an engineered human cell, which has reduced or eliminated surface expression of HLA-B protein relative to an unmodified cell, comprising a genetic modification in the HLA-B gene, wherein the genetic modification comprises an indel, a C to T substitution, or an A to G substitution within the genomic coordinates chosen from: (a) chr6:31355348-31355368; or chr6:31355347-31355367; chr6:31355182-31355202; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355432-31355452; chr6:31355340-31355360; chr6:31355576-31355596; chr6:31355410-31355430; chr6:31355419-31355439; chr6:31355414-31355434; or chr6:31355409-31355429; or (b) chr6:31355221-31355245; chr6:31355222-31355246; chr6:31355205-31355229; chr6:31355446-31355470; chr6:31356425-31356449; chr6:31355441-31355465; chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

Embodiment 25 is an engineered human cell, which has reduced or eliminated surface expression of HLA-A and HLA-B protein relative to an unmodified cell, comprising (i) a genetic modification in the HLA-A gene comprising an indel, a C to T substitution, or an A to G substitution within the genomic coordinates chosen from: (a) chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; chr6:29944026-29944046; chr6:29934330-29934350, chr6:29943115-29943135, chr6:29943135-29943155, chr6:29943140-29943160, chr6:29943590-29943610, chr6:29943824-29943844, chr6:29943858-29943878, chr6:29944478-29944498, and chr6:29944850-29944870; or (b) chr6:29942891-29942915; chr6:29942609-29942633; chr6:29944266-29944290; chr6:29942889-29942913; chr6:29944471-29944495; and chr6:29944470-29944494; and (ii) a genetic modification in the HLA-B gene comprising an indel, a C to T substitution, or an A to G substitution within the genomic coordinates chosen from: (a) chr6:31355348-31355368; or chr6:31355347-31355367; chr6:31355182-31355202; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355432-31355452; chr6:31355340-31355360; chr6:31355576-31355596; chr6:31355410-31355430; chr6:31355419-31355439; chr6:31355414-31355434; and chr6:31355409-31355429; or (b) chr6:31355221-31355245; chr6:31355222-31355246; chr6:31355205-31355229; chr6:31355446-31355470; chr6:31356425-31356449; chr6:31355441-31355465; chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

Embodiment 26 is the engineered cell of any one of embodiment 24 or 25, wherein the genetic modification in the HLA-B comprises an indel, a C to T substitution, or an A to G substitution within the genomic coordinates chosen from: (a) chr6:31355348-31355368; or (b) chr6:31355390-31355414; chr6:31355417-31355441; or chr6: 31355390-31355414.

Embodiment 27 is the engineered cell of any one of embodiments 24-26, wherein the genetic modification in the HLA-A or the genetic modification in the HLA-B comprises at least 2, at least 3, at least 4, at least 5, at least 6, at least 7, at least 8, at least 9, or at least 10 contiguous nucleotides within the genomic coordinates, or wherein the genetic modification comprises at least 5 contiguous nucleotides within the genomic coordinates.

Embodiment 28 is the engineered cell of any one of embodiments 24-27, wherein the genetic modification in the HLA-A or the genetic modification in the HLA-B comprises at least 6, 7, 8, 9, or 10 contiguous nucleotides within the genomic coordinates.

Embodiment 29 is the engineered cell of any one of embodiments 24-28, wherein the genetic modification in the HLA-A or the genetic modification in the HLA-B comprises at least one C to T substitution or at least one A to G substitution within the genomic coordinates.

Embodiment 30 is the engineered cell of any one of embodiments 1-29, wherein the HLA-B expression is reduced or eliminated by a gene editing system that binds to an HLA-B genomic target sequence comprising at least 5 contiguous nucleotides within the genomic coordinates chosen from: chr6:31355348-31355368; or chr6:31355347-31355367; chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429.

Embodiment 31 is the engineered cell of any one of embodiments 1-30, wherein the HLA-B expression is reduced or eliminated by a gene editing system that binds to an HLA-B genomic target sequence comprising at least 5 contiguous nucleotides within the genomic coordinates chosen from: chr6:31355221-31355245; chr6:31355222-31355246; chr6:31355205-31355229; chr6:31355446-31355470; chr6:31356425-31356449; chr6:31355441-31355465; chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

Embodiment 32 is the engineered cell of any one of embodiments 1-2, 5-23, and 25-31, wherein HLA-A expression is reduced or eliminated by a gene editing system that binds to an HLA-A genomic target sequence comprising at least 5 contiguous nucleotides within the genomic coordinates chosen from: chr6:29942891-29942915; chr6:29942609-29942633; chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; chr6:29944026-29944046; chr6:29934330-29934350, chr6:29943115-29943135, chr6:29943135-29943155, chr6:29943140-29943160, chr6:29943590-29943610, chr6:29943824-29943844, chr6:29943858-29943878, and chr6:29944478-29944498, chr6:29944850-29944870.

Embodiment 33 is the engineered cell of any one of embodiments 1, 2, 5-23, and 25-32, wherein HLA-A expression is reduced or eliminated by a gene editing system that binds to an HLA-A genomic target sequence comprising at least 5 contiguous nucleotides within the genomic coordinates chosen from: chr6:29942891-29942915; chr6:29942609-29942633; chr6:29944266-29944290; chr6:29942889-29942913; chr6:29944471-29944495; and chr6:29944470-29944494.

Embodiment 34 is the engineered cell of any one of embodiments 1, 2, 5-23, and 25-33, wherein HLA-A expression is reduced or eliminated by a gene editing system that binds to an HLA-A genomic target sequence comprising at least 5 contiguous nucleotides within the genomic coordinates chosen from: chr6:29942891-29942915 or chr6:29942609-29942633.

Embodiment 35 is the engineered cell of any one of embodiments 30-34, wherein the HLA-A genomic target sequence or the HLA-B genomic target sequence comprises at least 10 contiguous nucleotides within the genomic coordinates.

Embodiment 36 is the engineered cell of any one of embodiments 30-35, wherein the HLA-A genomic target sequence or the HLA-B genomic target sequence comprises at least 15 contiguous nucleotides within the genomic coordinates.

Embodiment 37 is the engineered cell of any one of embodiments 30-36, wherein the HLA-A genomic target sequence or the HLA-B genomic target sequence comprises at least 17, 18, 19, or 20 contiguous nucleotides within the genomic coordinates.

Embodiment 38 is the engineered cell of any one of embodiments 30-36, wherein the HLA-A genomic target sequence or the HLA-B genomic target sequence comprises at least 17, 18, 19, 20, 21, 22, 23, 24 or 25 contiguous nucleotides within the genomic coordinates.

Embodiment 39 is the engineered cell of any one of embodiments 1-38, wherein the cell is homozygous for HLA-C.

Embodiment 40 is the engineered cell of any one of embodiments 1-39, wherein the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*08:01; HLA-C*03:02; HLA-C*06:02; HLA-C*16:01; HLA-C*12:03; HLA-C*04:01; HLA-C*15:02; HLA-C*07:01; HLA-C*03:04; HLA-C*12:03; HLA-C*02:10; HLA-C*05:01; HLA-C*12:02; HLA-C*14:02; HLA-C*06:02; HLA-C*04:01; HLA-C*03:03; HLA-C*07:04; HLA-C*07:04; HLA-C*04:01; HLA-C*17:01; HLA-C*01:02; and HLA-C*02:02.

Embodiment 41 is the engineered cell of any one of embodiments 1-40, wherein the HLA-C allele is HLA-C*07:02.

Embodiment 42 is the engineered cell of any one of embodiments 1-40, wherein the HLA-C allele is HLA-C*07:01.

Embodiment 43 is the engineered cell of any one of embodiments 1-40, wherein the HLA-C allele is HLA-C*05:01.

Embodiment 44 is the engineered cell of any one of embodiments 1-40, wherein the HLA-C allele is HLA-C*04:01.

Embodiment 45 is the engineered cell of any one of embodiments 1-40, wherein the HLA-C allele is HLA-C*06:02.

Embodiment 46 is the engineered cell of any one of embodiments 3-24 and 26-45, wherein the engineered cell is homozygous for HLA-A, the HLA-A allele is selected from any one of the following HLA-A alleles: HLA-A*02:01; HLA-A*01:01; HLA-A*03:01; HLA-A*11:01; HLA-A*26:01; HLA-A*68:01; HLA-A*29:02; HLA-A*31:01; HLA-A*32:01; HLA-A*30:02; HLA-A*25:01; HLA-A*33:01; HLA-A*02:02; HLA-A*74:01; HLA-A*02:02; HLA-A*29:01; HLA-A*02:03; HLA-A*02:05; HLA-A*24:07; HLA-A*11:02; HLA-A*36:01; HLA-A*02:22; HLA-A*34:02; HLA-A*01:03; HLA-A*24:02; HLA-A*02:07; HLA-A*23:01; HLA-A*30:01; HLA-A*33:03; HLA-A*02:06; HLA-A*34:02; and HLA-A*68:02.

Embodiment 47 is the engineered cell of any one of embodiments 3-24 and 26-45, wherein the engineered cell is homozygous for HLA-A and wherein the engineered cell is homozygous for HLA-C wherein the HLA-A allele is selected from any one of the following HLA-A alleles: HLA-A*02:01; HLA-A*01:01; HLA-A*03:01; HLA-A*11:01; HLA-A*26:01; HLA-A*68:01; HLA-A*29:02; HLA-A*31:01; HLA-A*32:01; HLA-A*30:02; HLA-A*25:01; HLA-A*33:01; HLA-A*02:02; HLA-A*74:01; HLA-A*02:02; HLA-A*29:01; HLA-A*02:03; HLA-A*02:05; HLA-A*24:07; HLA-A*11:02; HLA-A*36:01; HLA-A*02:22; HLA-A*34:02; HLA-A*01:03; HLA-A*24:02; HLA-A*02:07; HLA-A*23:01; HLA-A*30:01; HLA-A*33:03; HLA-A*02:06; HLA-A*34:02; and HLA-A*68:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*08:01; HLA-C*03:02; HLA-C*16:01; HLA-C*15:02; HLA-C*03:04; HLA-C*12:03; HLA-C*02:10; HLA-C*05:01; HLA-C*12:02; HLA-C*14:02; HLA-C*04:01; HLA-C*03:03; HLA-C*07:04; HLA-C*17:01; HLA-C*01:02; and HLA-C*02:02.

Embodiment 48 is the engineered cell of any one of embodiments 1-47, wherein the cell has reduced or eliminated surface expression of MHC class II protein.

Embodiment 49 is the engineered cell of any one of embodiments 1-48, wherein the cell has a genetic modification of a gene selected from CIITA, HLA-DR, HLA-DQ, HLA-DP, RFX5, RFXB/ANK, RFXAP, CREB, NF-YA, NF-YB, and NF-YC.

Embodiment 50 is the engineered cell of any one of embodiments 1-49, wherein the cell has a genetic modification in the CIITA gene.

Embodiment 51 is the engineered cell of any one of embodiments 1-50, wherein the cell has reduced or eliminated surface expression of TRAC protein.

Embodiment 52 is the engineered cell of any one of embodiments 1-51, wherein the cell has reduced or eliminated surface expression of TRBC protein.

Embodiment 53 is the engineered cell of any one of embodiments 1-52, wherein the genetic modification comprises at least 2, at least 3, at least 4, at least 5, at least 6, at least 7, at least 8, at least 9, or at least 10 contiguous nucleotides within the genomic coordinates, or wherein the genetic modification comprises at least 5 contiguous nucleotides within the genomic coordinates.

Embodiment 54 is the engineered cell of any one of embodiments 1-53, wherein the genetic modification comprises at least 6, 7, 8, 9, or 10 contiguous nucleotides within the genomic coordinates.

Embodiment 55 is the engineered cell of any one of embodiments 1-54, wherein the genetic modification comprises an indel.

Embodiment 56 is the engineered cell of any one of embodiments 1-55, wherein the genetic modification comprises at least one C to T substitution or at least one A to G substitution within the genomic coordinates.

Embodiment 57 is a pharmaceutical composition comprising the engineered cell of any one of embodiments 1-56.

Embodiment 58 is a population of cells comprising the engineered cell of any one of embodiments 1-57.

Embodiment 59 is a pharmaceutical composition comprising the population of cells of embodiment 58.

Embodiment 60 is the population of embodiment 58 or the pharmaceutical composition of embodiment 59, wherein the population of cells is at least 65%, at least 70%, at least 80%, at least 90%, at least 91%, at least 92%, at least 93%, at least 94%, at least 95%, at least 96%, at least 97%, at least 98%, or at least 99% HLA-A negative or HLA-B negative as measured by flow cytometry.

Embodiment 61 is the population or pharmaceutical composition of any one of embodiments 58-60, wherein at least 65%, at least 70%, at least 80%, at least 85%, at least 90%, at least 95%, at least 96%, at least 97%, at least 98%, or at least 99% of the population of cells comprises the genetic modification in the HLA-A gene or the genetic modification in the HLA-B gene, as measured by next-generation sequencing (NGS).

Embodiment 62 is the population or pharmaceutical composition of any one of embodiments 58-61, wherein the population of cells is at least 94%, at least 95%, at least 96%, at least 97%, at least 98%, or at least 99% CIITA negative as measured by flow cytometry.

Embodiment 63 is the population or pharmaceutical composition of any one of embodiments 58-62, wherein the population of cells is at least 95%, at least 97%, at least 98%, at least 99%, or at least 99.5% endogenous TCR protein negative as measured by flow cytometry.

Embodiment 64 is a method of administering the engineered cell, population of cells, pharmaceutical composition of any one of embodiments 1-63 to a subject in need thereof.

Embodiment 65 is a method of administering the engineered cell, population of cells, or pharmaceutical composition of any one of embodiments 1-63 to a subject as an adoptive cell transfer (ACT) therapy.

Embodiment 66 is a method of treating a disease or disorder comprising administering the engineered cell, population of cells, or pharmaceutical composition of any one of embodiments 1-63 to a subject in need thereof.

Embodiment 67 is a composition, comprising an HLA-B guide RNA, wherein the HLA-B guide RNA comprises: i. a guide sequence selected from SEQ ID NOs: 165, 166, 177, 13, 74, 1-12, 14-73, 75-91, 101-164, 167-176, 178-185; ii. at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected form SEQ ID NOs: 101-185; iii. a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185; iv. a guide sequence that binds a target site comprising a genomic region listed in Table 2 or 3; or v. a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3.

Embodiment 68 is a composition, comprising an HLA-B guide RNA and an HLA-A guide RNA, wherein the HLA-B guide RNA comprises: i. a guide sequence selected from SEQ ID NOs: 165, 166, 177, 13, 74, 1-12, 14-73, 75-91 and 101-164, 167-176, 178-185; ii. at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected form SEQ ID NOs: 101-185; iii. a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185; iv. a guide sequence that binds a target site comprising a genomic region listed in Table 2 or 3; or v. a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3, and wherein the HLA-A guide RNA comprises: i. a guide sequence selected from SEQ ID Nos: 576, 571, 301-570, 572-575, 577-590; or ii. at least 17, 18, 19, or 20 contiguous nucleotides of a sequence selected from SEQ ID NOs: 301-428 and 463-511; or at least 17, 18, 19, 20, 21, 22, 23, or 24 contiguous nucleotides of a sequence selected form SEQ ID NOs: 429-462 and 512-590; or iii. a guide sequence at least 95%, 90%, or 85% identical to a sequence selected from SEQ ID NOs: 301-428 and 463-511; or a guide sequence at least 95%, 90%, 85%, 80%, 75%, or 70% identical to a sequence selected from SEQ ID NOs: 512-590; or iv. a guide sequence that binds a target site comprising a genomic region listed in Tables 4-7; or v. a guide sequence that is complementary to at least 17, 18, 19, 20, 21, 22, 23, or 24 contiguous nucleotides of a genomic region listed in Tables 4-7.

Embodiment 69 is a method of making an engineered human cell, which has reduced or eliminated surface expression of HLA-B protein relative to an unmodified cell, wherein the cell is homozygous for HLA-A and homozygous for HLA-C, comprising: contacting a cell with a composition comprising (i) an HLA-B guide RNA and (ii) optionally an RNA-guided DNA binding agent or a nucleic acid encoding an RNA-guided DNA binding agent, wherein the HLA-B guide RNA comprises: i. a guide sequence selected from SEQ ID NOs: 165, 166, 177, 13, 74, 1-12, 14-73, 75-91, 101-164, 167-176, 178-185; ii. at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected form SEQ ID NOs: 101-185; iii. a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185; iv. a guide sequence that binds a target site comprising a genomic region listed in Table 2 or 3; or v. a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3.

Embodiment 70 is a method of reducing surface expression of HLA-B protein in a human cell relative to an unmodified cell, comprising contacting a cell with a composition comprising (i) an HLA-B guide RNA and (ii) optionally an RNA-guided DNA binding agent or a nucleic acid encoding an RNA-guided DNA binding agent, wherein the HLA-B guide RNA comprises: i. a guide sequence selected from SEQ ID NOs: 165, 166, 177, 13, 74, 1-12, 14-73, 75-91 and 101-164, 167-176, 178-185; ii. at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected form SEQ ID NOs; and 101-185; iii. a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185; iv. a guide sequence that binds a target site comprising a genomic region listed in Table 2 or 3; or v. a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3.

Embodiment 71 is a method of making an engineered human cell, which has reduced or eliminated surface expression of HLA-A and HLA-B protein relative to an unmodified cell, wherein the cell is homozygous for HLA-C, comprising: (a) contacting a cell with a first composition comprising an HLA-B guide RNA and optionally an RNA-guided DNA binding agent or a nucleic acid encoding an RNA-guided DNA binding agent, wherein the HLA-B guide RNA comprises: i. a guide sequence selected from SEQ ID NOs: 165, 166, 177, 13, 74, 1-12, 14-73, 75-91 and 101-164, 167-176, 178-185; or ii. at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected form SEQ ID NOs: 101-185; or iii. a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185; iv. a guide sequence that binds a target site comprising a genomic region listed in Table 2 or 3; or v. a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3; and (b) contacting a cell with a second composition comprising an HLA-A guide RNA and optionally an RNA-guided DNA binding agent or a nucleic acid encoding an RNA-guided DNA binding agent, wherein the HLA-A guide RNA comprises: i. a guide sequence selected from SEQ ID Nos: 576, 571, 301-570, 572-575, 577-590; or ii. at least 17, 18, 19, or 20 contiguous nucleotides of a sequence selected from SEQ ID NOs: 301-428 and 463-511; or at least 17, 18, 19, 20, 21, 22, 23, or 24 contiguous nucleotides of a sequence selected form SEQ ID NOs: 429-462 and 512-590; or iii. a guide sequence at least 95%, 90%, or 85% identical to a sequence selected from SEQ ID NOs: 301-428 and 463-511; or a guide sequence at least 95%, 90%, 85%, 80%, 75%, or 70% identical to a sequence selected from SEQ ID NOs: 512-590; or iv. a guide sequence that binds a target site comprising a genomic region listed in Tables 4-7; or a guide sequence that is complementary to at least 17, 18, 19, 20, 21, 22, 23, or 24 contiguous nucleotides of a genomic region listed in Tables 4-7.

Embodiment 72 is a method of reducing surface expression of HLA-A protein and HLA-B protein in a human cell relative to an unmodified cell, comprising (a) contacting a cell with a first composition comprising an HLA-B guide RNA and optionally an RNA-guided DNA binding agent or a nucleic acid encoding an RNA-guided DNA binding agent, wherein the HLA-B guide RNA comprises: i. a guide sequence selected from SEQ ID NOs: 165, 166, 177, 13, 74, 1-12, 14-73, 75-91 and 101-164, 167-176, 178-185; ii. at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected form SEQ ID NOs; and 101-185; iii. a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185; iv. a guide sequence that binds a target site comprising a genomic region listed in Table 2 or 3; or v. a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3; and (b) contacting a cell with a second composition comprising an HLA-A guide RNA and optionally an RNA-guided DNA binding agent or a nucleic acid encoding an RNA-guided DNA binding agent, wherein the HLA-A guide RNA comprises: i. a guide sequence selected from SEQ ID Nos: 576, 571, 301-570, 572-575, 577-590; or ii. at least 17, 18, 19, or 20 contiguous nucleotides of a sequence selected from SEQ ID NOs: 301-428 and 463-511; or at least 17, 18, 19, 20, 21, 22, 23, or 24 contiguous nucleotides of a sequence selected form SEQ ID NOs: 429-462 and 512-590; or iii. a guide sequence at least 95%, 90%, or 85% identical to a sequence selected from SEQ ID NOs: 301-428 and 463-511; or a guide sequence at least 95%, 90%, 85%, 80%, 75%, or 70% identical to a sequence selected from SEQ ID NOs: 429-462 and 512-590; or iv. a guide sequence that binds a target site comprising a genomic region listed in Tables 4-7; or v. a guide sequence that is complementary to at least 17, 18, 19, 20, 21, 22, 23, or 24 contiguous nucleotides of a genomic region listed in Tables 4-7.

Embodiment 73 is the engineered cell, population of cells, pharmaceutical composition, or method of any one of embodiments 1-72, wherein the RNA-guided DNA-binding agent or nucleic acid encoding the RNA-guided DNA binding agent is SpyCas9, and the HLA-B guide RNA comprises: (i) a guide sequence selected from SEQ ID NOs: 13, 74, 1-12, 14-73, 75-91; or (ii) a guide sequence that is at least 17, 18, 19, 20, 21, 22, 23, or 24 contiguous nucleotides of a sequence selected from SEQ ID NOs: 1-91; or (iii) a guide sequence that is at least 17, 18, 19, or 20 contiguous nucleotides of a sequence selected from SEQ ID NOs: 1-91; or (iv) a guide sequence that binds a target site comprising a genomic region listed in Table 2; or (v) a guide sequence that is complementary to at least 17, 18, 19, or 20 contiguous nucleotides of a genomic region listed in Table 2; or (vi) a guide sequence at least 95%, 90%, or 85% identical to a sequence selected from SEQ ID NOs: 1-91.

Embodiment 74 is the engineered cell, population of cells, pharmaceutical composition, or method of any one of embodiments 1-72, wherein the RNA-guided DNA-binding agent or nucleic acid encoding the RNA-guided DNA binding agent is NmeCas9, and the HLA-B guide RNA comprises: (i) a guide sequence selected from SEQ ID NOs: 165, 166, 177, 101-164, 167-176, and 178-185; or (ii) a guide sequence that is at least 17, 18, 19, 20, 21, 22, 23, or 24 contiguous nucleotides of a sequence selected from SEQ ID NOs: 101-185; or (iii) a guide sequence that is at least 17, 18, 19, 20, 21, 22, 23, or 24 contiguous nucleotides of a sequence selected from SEQ ID NOs: 101-185; or (iv) a guide sequence that binds a target site comprising a genomic region listed in Table 3; or (v) a guide sequence that is complementary to at least 17, 18, 19, 20, 21, 22, 23, or 24 contiguous nucleotides of a genomic region listed in Table 3; or (vi) a guide sequence at least 95%, 90%, 85%, 80%, 75%, or 70% identical to a sequence selected from SEQ ID NOs: 101-185.

Embodiment 75 is the engineered cell, population of cells, pharmaceutical composition, or method of any one of embodiments 1-72, wherein the RNA-guided DNA-binding agent or nucleic acid encoding the RNA-guided DNA binding agent is NmeCas9, and the HLA-B guide RNA comprises: (i) a guide sequence selected from SEQ ID NOs: 165, 166, 163, 164, 169, and 177; or (ii) a guide sequence that is at least 17, 18, 19, 20, 21, 22, 23, or 24 contiguous nucleotides of a sequence selected from SEQ ID NOs: 165, 166, 163, 164, and 177; or (iii) a guide sequence at least 95%, 90%, 85%, 80%, 75%, or 70% identical to a sequence selected from SEQ ID NOs: 165, 166, 163, 164, and 177.

Embodiment 76 is the composition or method of any one of embodiments 67-75, wherein the HLA-B guide RNA or the HLA-A guide RNA comprises at least one modification.

Embodiment 77 is the composition or method of any one of embodiments 67-76, wherein the HLA-B guide RNA or the HLA-A guide RNA comprises at least one modification, wherein the at least one modification includes a 2′-O-methyl (2′-O-Me) modified nucleotide.

Embodiment 78 is the composition or method of any one of embodiments 67-77, wherein the HLA-B guide RNA or the HLA-A guide RNA comprises at least one modification, comprising a phosphorothioate (PS) bond between nucleotides.

Embodiment 79 is the composition or method of any one of embodiments 67-78, wherein the HLA-B guide RNA or the HLA-A guide RNA comprises at least one modification, comprising a 2′-fluoro (2′-F) modified nucleotide.

Embodiment 80 is the composition or method of any one of embodiments 67-79, wherein the HLA-B guide RNA or the HLA-A guide RNA comprises at least one modification, comprising a modification at one or more of the first five nucleotides at the 5′ end of the guide RNA.

Embodiment 81 is the composition or method of any one of embodiments 67-80, wherein the HLA-B guide RNA or the HLA-A guide RNA comprises at least one modification, comprising a modification at one or more of the last five nucleotides at the 3′ end of the guide RNA.

Embodiment 82 is the composition or method of any one of embodiments 67-81, wherein the HLA-B guide RNA or the HLA-A guide RNA comprises at least one modification, comprising a PS bond between the first four nucleotides of the guide RNA.

Embodiment 83 is the composition or method of any one of embodiments 67-82, wherein the HLA-B guide RNA or the HLA-A guide RNA comprises at least one modification, comprising a PS bond between the last four nucleotides of the guide RNA.

Embodiment 84 is the composition or method of any one of embodiments 67-83, wherein the HLA-B guide RNA or the HLA-A guide RNA comprises at least one modification, comprising a 2′-O-Me modified nucleotide at the first three nucleotides at the 5′ end of the guide RNA.

Embodiment 85 is the composition or method of any one of embodiments 67-84, wherein the HLA-B guide RNA or the HLA-A guide RNA comprises at least one modification, comprising a 2′-O-Me modified nucleotide at the last three nucleotides at the 3′ end of the guide RNA.

Embodiment 86 is the method of any one of embodiments 67-85, further comprising reducing or eliminating the surface expression of MHC class II protein in the cell relative to an unmodified cell, for example by contacting the cell with a gene editing system targeting a gene selected from CIITA, HLA-DR, HLA-DQ, HLA-DP, RFX5, RFXB/ANK, RFXAP, CREB, NF-YA, NF-YB, and NF-YC.

Embodiment 87 is the method of any one of embodiments 67-86, further comprising contacting the cell with a CIITA guide RNA.

Embodiment 88 is the method of any one of embodiments 67-87, further comprising reducing or eliminating the surface expression of a TCR protein in the cell relative to an unmodified cell.

Embodiment 89 is the method of any one of embodiments 67-88, further comprising contacting the cell with an exogenous nucleic acid.

Embodiment 90 is the method of embodiment 89, further comprising contacting the cell with an exogenous nucleic acid encoding a targeting receptor.

Embodiment 91 is the method of embodiment 89, further comprising contacting the cell with an exogenous nucleic acid encoding a polypeptide that is secreted by the cell.

Embodiment 92 is the method of embodiment 89, further comprising contacting the cell with a DNA-dependent protein kinase inhibitor (DNAPKi).

Embodiment 93 is the method of embodiment 92, wherein the DNAPKi is Compound 1.

Embodiment 94 is the engineered cell, population of cells, pharmaceutical composition, or method of any one of embodiments 1-93, wherein the cell is an allogeneic cell.

Embodiment 95 is the engineered cell, population of cells, pharmaceutical composition, or method of any one of embodiments 1-94, wherein the cell is a primary cell.

Embodiment 96 is the engineered cell, population of cells, pharmaceutical composition, or method of any one of embodiments 1-94, wherein the cell is a CD4+ T cell.

Embodiment 97 is the engineered cell, population of cells, pharmaceutical composition, or method of any one of embodiments 1-94, wherein the cell is a CD8+ T cell.

Embodiment 98 is the engineered cell, population of cells, pharmaceutical composition, or method of any one of embodiments 1-94, wherein the cell is a memory T cell.

Embodiment 99 is the engineered cell, population of cells, pharmaceutical composition, or method of any one of embodiments 1-94, wherein the cell is a B cell.

Embodiment 100 is the engineered cell, population of cells, pharmaceutical composition, or method of any one of embodiments 1-94, wherein the cell is a plasma B cell.

Embodiment 101 is the engineered cell, population of cells, pharmaceutical composition, or method of any one of embodiments 1-94, wherein the cell is a memory B cell.

Embodiment 102 is the engineered cell, population of cells, pharmaceutical composition, or method of any one of embodiments 1-94, wherein the cell is a natural killer (NK) cell.

Embodiment 103 is the engineered cell, population of cells, pharmaceutical composition, or method of any one of embodiments 1-94, wherein the cell is a macrophage.

Embodiment 104 is the engineered cell, population of cells, pharmaceutical composition, or method of any one of embodiments 1-94, wherein the cell is a stem cell.

Embodiment 105 is the engineered cell, population of cells, pharmaceutical composition, or method of any one of embodiments 1-94, wherein the cell is a pluripotent stem cell (PSC).

Embodiment 106 is the engineered cell, population of cells, pharmaceutical composition, or method of any one of embodiments 1-94, wherein the cell is a hematopoietic stem cell (HSC).

Embodiment 107 is the engineered cell, population of cells, pharmaceutical composition, or method of any one of embodiments 1-94, wherein the cell is an induced pluripotent stem cell (iPSC).

Embodiment 108 is the engineered cell, population of cells, pharmaceutical composition, or method of any one of embodiments 1-94, wherein the cell is a mesenchymal stem cell (MSC).

Embodiment 109 is the engineered cell, population of cells, pharmaceutical composition, or method of any one of embodiments 1-94, wherein the cell is a neural stem cell (NSC).

Embodiment 110 is the engineered cell, population of cells, pharmaceutical composition, or method of any one of embodiments 1-94, wherein the cell is a limbal stem cell (LSC).

Embodiment 111 is the engineered cell, population of cells, pharmaceutical composition, or method of any one of embodiments 1-94, wherein the cell is a progenitor cell, e.g. an endothelial progenitor cell or a neural progenitor cell.

Embodiment 112 is the engineered cell, population of cells, pharmaceutical composition, or method of any one of embodiments 1-94, wherein the cell is a tissue-specific primary cell.

Embodiment 113 is the engineered cell, population of cells, pharmaceutical composition, or method of any one of embodiments 1-94, wherein the cell is a chosen from: chondrocyte, myocyte, and keratinocyte.

Embodiment 114 is the engineered cell, population of cells, pharmaceutical composition, or method of any one of embodiments 1-94, wherein the cell is an activated cell.

Embodiment 115 is the engineered cell, population of cells, pharmaceutical composition, or method of any one of embodiments 1-94, wherein the cell is a non-activated cell.

Embodiment 116 is the engineered cell, population of cells, pharmaceutical composition, or method of any one of embodiments 1-115, comprising an exogenous nucleic acid encoding a polypeptide that is secreted by the cell or contacting the cell with said exogenous nucleic acid, wherein the secreted polypeptide is an antibody or antibody fragment.

Embodiment 117 is the engineered cell, population of cells, pharmaceutical composition, or method of any one of embodiments 1-116, comprising an exogenous nucleic acid encoding a polypeptide that is secreted by the cell or contacting the cell with said exogenous nucleic acid, wherein the secreted polypeptide is a full-length IgG antibody.

Embodiment 118 is the engineered cell, population of cells, pharmaceutical composition, or method of any one of embodiments 1-116, comprising an exogenous nucleic acid encoding a polypeptide that is secreted by the cell or contacting the cell with said exogenous nucleic acid, wherein the secreted polypeptide is a single chain antibody.

Embodiment 119 is the engineered cell, population of cells, pharmaceutical composition, or method of any one of embodiments 1-118, comprising an exogenous nucleic acid encoding a polypeptide that is secreted by the cell or contacting the cell with said exogenous nucleic acid, wherein the secreted polypeptide is a neutralizing antibody.

Embodiment 120 is the engineered cell, population of cells, pharmaceutical composition, or method of any one of embodiments 1-115, comprising an exogenous nucleic acid encoding a polypeptide that is secreted by the cell or contacting the cell with said exogenous nucleic acid, wherein the secreted polypeptide is an enzyme.

Embodiment 121 is the engineered cell, population of cells, pharmaceutical composition, or method of any one of embodiments 1-115, comprising an exogenous nucleic acid encoding a polypeptide that is secreted by the cell or contacting the cell with said exogenous nucleic acid, wherein the secreted polypeptide is a cytokine.

Embodiment 122 is the engineered cell, population of cells, pharmaceutical composition, or method of any one of embodiments 1-121, comprising an exogenous nucleic acid encoding a polypeptide that is secreted by the cell or contacting the cell with said exogenous nucleic acid, wherein the secreted polypeptide is a fusion protein.

Embodiment 123 is the engineered cell, population of cells, pharmaceutical composition, or method of any one of embodiments 1-122, comprising an exogenous nucleic acid encoding a polypeptide that is secreted by the cell or contacting the cell with said exogenous nucleic acid, wherein the secreted polypeptide comprises a soluble receptor.

Embodiment 124 is the engineered cell, population of cells, pharmaceutical composition, or method of any one of embodiments 1-115, comprising an exogenous nucleic acid encoding a targeting receptor or contacting the cell with an exogenous nucleic acid encoding a targeting receptor, wherein the targeting receptor is a T cell receptor (TCR).

Embodiment 125 is the engineered cell, population of cells, pharmaceutical composition, or method of any one of embodiments 1-115, comprising an exogenous nucleic acid encoding a targeting receptor or contacting the cell with an exogenous nucleic acid encoding a targeting receptor, wherein the targeting receptor is a genetically modified TCR.

Embodiment 126 is the engineered cell, population of cells, pharmaceutical composition, or method of any one of embodiments 1-115, comprising an exogenous nucleic acid encoding a targeting receptor or contacting the cell with an exogenous nucleic acid encoding a targeting receptor, wherein the targeting receptor is a WT1 TCR.

Embodiment 127 is the engineered cell, population of cells, pharmaceutical composition, or method of any one of embodiments 1-115, comprising an exogenous nucleic acid encoding a targeting receptor or contacting the cell with an exogenous nucleic acid encoding a targeting receptor, wherein the targeting receptor is a CAR.

Embodiment 128 is the engineered cell, population of cells, pharmaceutical composition, or method of any one of embodiments 1-115, comprising an exogenous nucleic acid encoding a targeting receptor or contacting the cell with an exogenous nucleic acid encoding a targeting receptor, wherein the targeting receptor is a universal CAR.

Embodiment 129 is the engineered cell, population of cells, pharmaceutical composition, or method of any one of embodiments 1-127, comprising an exogenous nucleic acid encoding a targeting receptor or contacting the cell with an exogenous nucleic acid encoding a targeting receptor, wherein the targeting receptor is an anti-CD30 CAR.

Embodiment 130 is the engineered cell, population of cells, pharmaceutical composition, or method of any one of embodiments 1-129, comprising an exogenous nucleic acid encoding a targeting receptor or contacting the cell with an exogenous nucleic acid encoding a targeting receptor, wherein the targeting receptor is a proliferation-inducing ligand (APRIL).

Embodiment 131 is the engineered cell, population of cells, pharmaceutical composition, or method of any one of embodiments 1-129, wherein the cells are engineered with a gene editing system.

Embodiment 132 is the engineered cell, population of cells, pharmaceutical composition, or method of embodiment 131, wherein the gene editing system comprises a transcription activator-like effector nuclease (TALEN).

Embodiment 133 is the engineered cell, population of cells, pharmaceutical composition, or method of embodiment 131, wherein the gene editing system comprises a zinc finger nuclease.

Embodiment 134 is the engineered cell, population of cells, pharmaceutical composition, or method of embodiment 131, wherein the gene editing system comprises an RNA-guided DNA binding agent or a nucleic acid encoding an RNA-guided DNA binding agent.

Embodiment 135 is the engineered cell, population of cells, pharmaceutical composition, or method of any one of embodiments 67-134, wherein the RNA-guided DNA-binding agent or the RNA-guided DNA-binding agent encoded by the nucleic acid comprises a Cas9 protein.

Embodiment 136 is the engineered cell, population of cells, pharmaceutical composition, or method of any one of embodiments 67-135, wherein the RNA-guided DNA-binding agent or the RNA-guided DNA-binding agent encoded by the nucleic acid is S. pyogenes Cas9.

Embodiment 137 is the engineered cell, population of cells, pharmaceutical composition, or method of any one of embodiments 67-135, wherein the RNA-guided DNA-binding agent or the RNA-guided DNA-binding agent encoded by the nucleic acid is N. meningitidis Cas9, optionally Nme2Cas9.

Embodiment 138 is the engineered cell, population of cells, pharmaceutical composition, or method of any one of embodiments 67-135, wherein the RNA-guided DNA-binding agent or the RNA-guided DNA-binding agent encoded by the nucleic acid is S. thermophilus Cas9.

Embodiment 139 is the engineered cell, population of cells, pharmaceutical composition, or method of any one of embodiments 67-135, wherein the RNA-guided DNA-binding agent or the RNA-guided DNA-binding agent encoded by the nucleic acid is S. aureus Cas9.

Embodiment 140 is the engineered cell, population of cells, pharmaceutical composition, or method of any one of embodiments 67-135, wherein the RNA-guided DNA-binding agent or the RNA-guided DNA-binding agent encoded by the nucleic acid is Cpf1 from F. novicida.

Embodiment 141 is the engineered cell, population of cells, pharmaceutical composition, or method of any one of embodiments 67-135, wherein the RNA-guided DNA-binding agent or the RNA-guided DNA-binding agent encoded by the nucleic acid is Cpf1 from Acidaminococcus sp.

Embodiment 142 is the engineered cell, population of cells, pharmaceutical composition, or method of any one of embodiments 67-135, wherein the RNA-guided DNA-binding agent or the RNA-guided DNA-binding agent encoded by the nucleic acid is Cpf1 from Lachnospiraceae bacterium ND2006.

Embodiment 143 is the engineered cell, population of cells, pharmaceutical composition, or method of any one of embodiments 67-135, wherein the RNA-guided DNA-binding agent or the RNA-guided DNA-binding agent encoded by the nucleic acid is Cas12a.

Embodiment 144 is the engineered cell, population of cells, pharmaceutical composition, or method of any one of embodiments 67-135, wherein the RNA-guided DNA-binding agent or the RNA-guided DNA-binding agent encoded by the nucleic acid is CasX.

Embodiment 145 is the engineered cell, population of cells, pharmaceutical composition, or method of any one of embodiments 67-135, wherein the RNA-guided DNA-binding agent or the RNA-guided DNA-binding agent encoded by the nucleic acid is Mad7 nuclease.

Embodiment 146 is the engineered cell, population of cells, pharmaceutical composition, or method of any one of embodiments 67-135, wherein the RNA-guided DNA-binding agent or the RNA-guided DNA-binding agent encoded by the nucleic acid is an ARCUS nucleases.

Embodiment 147 is the engineered cell, population of cells, pharmaceutical composition, or method of any one of embodiments 67-135, wherein the RNA-guided DNA-binding agent or the RNA-guided DNA-binding agent encoded by the nucleic acid is an A to G base editor.

Embodiment 148 is the engineered cell, population of cells, pharmaceutical composition, or method of any one of embodiments 67-135, wherein the RNA-guided DNA-binding agent or the RNA-guided DNA-binding agent encoded by the nucleic acid is a C to T base editor.

Embodiment 149 is the engineered cell, population of cells, pharmaceutical composition, or method of any one of embodiments 67-135, wherein the RNA-guided DNA-binding agent or the RNA-guided DNA-binding agent encoded by the nucleic acid comprises a cytidine deaminase and an RNA-guided nickase.

Embodiment 150 is the engineered cell, population of cells, pharmaceutical composition, or method of any one of embodiments 1-149, wherein the cell is engineered by a base editing system comprising a C to T base editor or an A to G base editor.

Embodiment 151 is the engineered cell, population of cells, pharmaceutical composition, or method of any one of the immediately preceding embodiment, wherein the base editing system comprises a polypeptide comprising a cytidine deaminase and an RNA-guided nickase, or a nucleic acid encoding the polypeptide.

Embodiment 152 is the engineered cell, population of cells, pharmaceutical composition, or method of embodiment 149 or 151 wherein the cytidine deaminase comprises APOBEC3A deaminase (A3A).

Embodiment 153 is the engineered cell, population of cells, pharmaceutical composition, or method of embodiment 151, wherein the polypeptide comprises an amino acid sequence that is at least 80%, 85%, 90%, 95%, 98%, or 100% identical to SEQ ID NO: 811 or 976.

Embodiment 154 is the engineered cell, population of cells, pharmaceutical composition, or method of embodiment 151, wherein the nucleic acid encoding the polypeptide comprises a sequence that is at least 80%, 85%, 90%, 95%, 98%, or 100% identical to SEQ ID NO: 804 or SEQ ID NO: 822.

Embodiment 155 is the engineered cell, population of cells, pharmaceutical composition, or method of any one of embodiment 148-154, wherein the base editing system further comprises a uracil glycosylase inhibitor (UGI) in a polypeptide different from the polypeptide comprising a cytidine deaminase and an RNA-guided nickase.

Embodiment 156 is the engineered cell, population of cells, pharmaceutical composition, or method of embodiment 148-152, wherein the polypeptide comprising the cytidine deaminase and the RNA-guided nickase further comprises a uracil glycosylase inhibitor (UGI).

Embodiment 157 is the engineered cell, population of cells, pharmaceutical composition, or method of embodiment 156, wherein the polypeptide comprises an amino acid sequence that is at least 80%, 85%, 90%, 95%, 98%, or 100% identical to any one of SEQ ID NO: 977, 978, 979, and 980.

Embodiment 158 is the engineered cell, population of cells, pharmaceutical composition, or method of any one of embodiments 67-157, wherein the HLA-A guide RNA or the HLA-B guide RNA is provided to the cell in a vector.

Embodiment 159 is the engineered cell, population of cells, pharmaceutical composition, or method of any one of embodiments 67-158, wherein the RNA-guided DNA binding agent is provided to the cell in a vector, optionally in the same vector as the HLA-A guide RNA or the HLA-B guide RNA.

Embodiment 160 is the engineered cell, population of cells, pharmaceutical composition, or method of any one of embodiments 87-159, wherein the exogenous nucleic acid is provided to the cell in a vector.

Embodiment 161 is the engineered cell, population of cells, pharmaceutical composition, or method of any one of embodiments 158-160, wherein the vector is a viral vector.

Embodiment 162 is the engineered cell, population of cells, pharmaceutical composition, or method of any one of embodiments 158-160, wherein the vector is a non-viral vector.

Embodiment 163 is the engineered cell, population of cells, pharmaceutical composition, or method of any one of embodiments 158-160, wherein the vector is a lentiviral vector.

Embodiment 164 is the engineered cell, population of cells, pharmaceutical composition, or method of any one of embodiments 158-160, wherein the vector is a retroviral vector.

Embodiment 165 is the engineered cell, population of cells, pharmaceutical composition, or method of any one of embodiments 158-160, wherein the vector is an AAV.

Embodiment 166 is the engineered cell, population of cells, pharmaceutical composition, or method of any one of embodiments 67-165, wherein the guide RNA is provided to the cell in a lipid nanoparticle (LNP).

Embodiment 167 is the engineered cell, population of cells, pharmaceutical composition, or method of any one of embodiments 67-166, wherein the guide RNA is provided to the cell in a same lipid nanoparticle (LNP) as an RNA-guided DNA binding agent.

Embodiment 168 is the engineered cell, population of cells, pharmaceutical composition, or method of any one of embodiments 87-167, wherein the exogenous nucleic acid is provided to the cell in a lipid nanoparticle (LNP).

Embodiment 169 is the engineered cell, population of cells, pharmaceutical composition, or method of any one of embodiments 87-168, wherein the exogenous nucleic acid is integrated into the genome of the cell.

Embodiment 170 is the engineered cell, population of cells, pharmaceutical composition, or method of any one of embodiments 87-169, wherein the exogenous nucleic acid is integrated into the genome of the cell by homologous recombination (HR).

Embodiment 171 is the engineered cell, population of cells, pharmaceutical composition, or method of any one of embodiments 87-170, wherein the exogenous nucleic acid is integrated into a safe harbor locus in the genome of the cell.

Embodiment 172 is the engineered cell, population of cells, pharmaceutical composition, or method of any one of embodiments 87-171, wherein the exogenous nucleic acid is integrated into the gene of the cell by nonhomologous end joining (NHEJ).

Embodiment 173 is the engineered cell, population of cells, pharmaceutical composition, composition, or method of any one of embodiments 67-172, wherein the HLA-B guide RNA comprises a guide sequence selected from SEQ ID NOs: 3, 13, 18, 32, 36, 39, 48-56, 58, 64-71, 73-74, 80-82, 86, and 88-91.

Embodiment 174 is the engineered cell, population of cells, pharmaceutical composition, composition, or method of any one of embodiments 67-172, wherein the HLA-B guide RNA comprises a guide sequence selected from SEQ ID NOs: 3, 13, 36, 39, 49-56, 64-71, 74, 80-82, 88, and 90-91.

Embodiment 175 is the engineered cell, population of cells, pharmaceutical composition, composition, or method of any one of embodiments 67-172, wherein the HLA-B guide RNA comprises a guide sequence selected from SEQ ID NOs: 13, 39, 49, 52, 65, 74, 82, and 91.

Embodiment 176 is the engineered cell, population of cells, pharmaceutical composition, composition, or method of any one of embodiments 67-172, wherein the HLA-B guide RNA comprises a guide sequence selected from SEQ ID NOs: 3, 39, and 49-52.

Embodiment 177 is the engineered cell, population of cells, pharmaceutical composition, composition, or method of any one of embodiments 67-172, wherein the HLA-B guide RNA comprises a guide sequence selected from SEQ ID NOs: 3, 36, 39, 49, 50, 51, and 52.

Embodiment 178 is the engineered cell, population of cells, pharmaceutical composition, composition, or method of any one of embodiments 67-172, wherein the HLA-B guide RNA comprises a guide sequence selected from SEQ ID NOs: 39, 49, and 52.

Embodiment 179 is the engineered cell, population of cells, pharmaceutical composition, composition, or method of any one of embodiments 67-172, wherein the HLA-B guide RNA comprises a guide sequence selected from SEQ ID NOs: 49, 52-54, 55, 56, 64, 65, 67-71, 73-74, 80-82, and 90.

Embodiment 180 is the engineered cell, population of cells, pharmaceutical composition, composition, or method of any one of embodiments 67-172, wherein the HLA-B guide RNA comprises a guide sequence selected from SEQ ID NOs: 49, 51, 74, 81, and 82.

Embodiment 181 is the engineered cell, population of cells, pharmaceutical composition, composition, or method of any one of embodiments 67-172, wherein the HLA-B guide RNA comprises a guide sequence selected from SEQ ID NOs: 101, 103, 106, 107, 114, 117, 118, 125-129, 137, 138, 141, 143, 144, 145, 159, 160, 163, 164, 165, 166, 169, 171, 172, 173, 176, 177, 178, 179, and 180.

Embodiment 182 is the engineered cell, population of cells, pharmaceutical composition, composition, or method of any one of embodiments 67-172, wherein the HLA-B guide RNA comprises a guide sequence selected from SEQ ID NOs: 65 and 74.

Embodiment 183 is the engineered cell, population of cells, pharmaceutical composition, composition, or method of any one of embodiments 67-172, wherein the HLA-B guide RNA comprises a guide sequence selected from SEQ ID NOs: 49, 52-54, 56, 64-65, 67-71, 73-74, 80-82, 88, and 90-91.

Embodiment 184 is the engineered cell, population of cells, pharmaceutical composition, composition, or method of any one of embodiments 67-172, wherein the HLA-B guide RNA comprises a guide sequence selected from SEQ ID NOs: 74, 82, and 91.

Embodiment 185 is the engineered cell, population of cells, pharmaceutical composition, composition, or method of any one of embodiments 67-172, wherein the HLA-B guide RNA comprises a guide sequence selected from SEQ ID NOs: 3, 13, 18, 32, 36, 39, 48-56, 58, 64-71, 73-74, 80-82, 86, and 88-90.

Embodiment 186 is the engineered cell, population of cells, pharmaceutical composition, composition, or method of any one of embodiments 67-172, wherein the HLA-B guide RNA comprises SEQ ID NO: 3.

Embodiment 187 is the engineered cell, population of cells, pharmaceutical composition, composition, or method of any one of embodiments 67-172, wherein the HLA-B guide RNA comprises SEQ ID NO: 13.

Embodiment 188 is the engineered cell, population of cells, pharmaceutical composition, composition, or method of any one of embodiments 67-172, wherein the HLA-B guide RNA comprises SEQ ID NO: 18.

Embodiment 189 is the engineered cell, population of cells, pharmaceutical composition, composition, or method of any one of embodiments 67-172, wherein the HLA-B guide RNA comprises SEQ ID NO: 32.

Embodiment 190 is the engineered cell, population of cells, pharmaceutical composition, composition, or method of any one of embodiments 67-172, wherein the HLA-B guide RNA comprises SEQ ID NO: 36.

Embodiment 191 is the engineered cell, population of cells, pharmaceutical composition, composition, or method of any one of embodiments 67-172, wherein the HLA-B guide RNA comprises SEQ ID NO: 39.

Embodiment 192 is the engineered cell, population of cells, pharmaceutical composition, composition, or method of any one of embodiments 67-172, wherein the HLA-B guide RNA comprises SEQ ID NO: 48.

Embodiment 193 is the engineered cell, population of cells, pharmaceutical composition, composition, or method of any one of embodiments 67-172, wherein the HLA-B guide RNA comprises SEQ ID NO: 49.

Embodiment 194 is the engineered cell, population of cells, pharmaceutical composition, composition, or method of any one of embodiments 67-172, wherein the HLA-B guide RNA comprises SEQ ID NO: 50.

Embodiment 195 is the engineered cell, population of cells, pharmaceutical composition, composition, or method of any one of embodiments 67-172, wherein the HLA-B guide RNA comprises SEQ ID NO: 51.

Embodiment 196 is the engineered cell, population of cells, pharmaceutical composition, composition, or method of any one of embodiments 67-172, wherein the HLA-B guide RNA comprises SEQ ID NO: 52.

Embodiment 197 is the engineered cell, population of cells, pharmaceutical composition, composition, or method of any one of embodiments 67-172, wherein the HLA-B guide RNA comprises SEQ ID NO: 53.

Embodiment 198 is the engineered cell, population of cells, pharmaceutical composition, composition, or method of any one of embodiments 67-172, wherein the HLA-B guide RNA comprises SEQ ID NO: 54.

Embodiment 199 is the engineered cell, population of cells, pharmaceutical composition, composition, or method of any one of embodiments 67-172, wherein the HLA-B guide RNA comprises SEQ ID NO: 55.

Embodiment 200 is the engineered cell, population of cells, pharmaceutical composition, composition, or method of any one of embodiments 67-172, wherein the HLA-B guide RNA comprises SEQ ID NO: 56.

Embodiment 201 is the engineered cell, population of cells, pharmaceutical composition, composition, or method of any one of embodiments 67-172, wherein the HLA-B guide RNA comprises SEQ ID NO: 58.

Embodiment 202 is the engineered cell, population of cells, pharmaceutical composition, composition, or method of any one of embodiments 67-172, wherein the HLA-B guide RNA comprises SEQ ID NO: 64.

Embodiment 203 is the engineered cell, population of cells, pharmaceutical composition, composition, or method of any one of embodiments 67-172, wherein the HLA-B guide RNA comprises SEQ ID NO: 65.

Embodiment 204 is the engineered cell, population of cells, pharmaceutical composition, composition, or method of any one of embodiments 67-172, wherein the HLA-B guide RNA comprises SEQ ID NO: 66.

Embodiment 205 is the engineered cell, population of cells, pharmaceutical composition, composition, or method of any one of embodiments 67-172, wherein the HLA-B guide RNA comprises SEQ ID NO: 67.

Embodiment 206 is the engineered cell, population of cells, pharmaceutical composition, composition, or method of any one of embodiments 67-172, wherein the HLA-B guide RNA comprises SEQ ID NO: 68.

Embodiment 207 is the engineered cell, population of cells, pharmaceutical composition, composition, or method of any one of embodiments 67-172, wherein the HLA-B guide RNA comprises SEQ ID NO: 69.

Embodiment 208 is the engineered cell, population of cells, pharmaceutical composition, composition, or method of any one of embodiments 67-172, wherein the HLA-B guide RNA comprises SEQ ID NO: 70.

Embodiment 209 is the engineered cell, population of cells, pharmaceutical composition, composition, or method of any one of embodiments 67-172, wherein the HLA-B guide RNA comprises SEQ ID NO: 71.

Embodiment 210 is the engineered cell, population of cells, pharmaceutical composition, composition, or method of any one of embodiments 67-172, wherein the HLA-B guide RNA comprises SEQ ID NO: 73.

Embodiment 211 is the engineered cell, population of cells, pharmaceutical composition, composition, or method of any one of embodiments 67-172, wherein the HLA-B guide RNA comprises SEQ ID NO: 74.

Embodiment 212 is the engineered cell, population of cells, pharmaceutical composition, composition, or method of any one of embodiments 67-172, wherein the HLA-B guide RNA comprises SEQ ID NO: 80.

Embodiment 213 is the engineered cell, population of cells, pharmaceutical composition, composition, or method of any one of embodiments 67-172, wherein the HLA-B guide RNA comprises SEQ ID NO: 81.

Embodiment 214 is the engineered cell, population of cells, pharmaceutical composition, composition, or method of any one of embodiments 67-172, wherein the HLA-B guide RNA comprises SEQ ID NO: 82.

Embodiment 215 is the engineered cell, population of cells, pharmaceutical composition, composition, or method of any one of embodiments 67-172, wherein the HLA-B guide RNA comprises SEQ ID NO: 86.

Embodiment 216 is the engineered cell, population of cells, pharmaceutical composition, composition, or method of any one of embodiments 67-172, wherein the HLA-B guide RNA comprises SEQ ID NO: 88.

Embodiment 217 is the engineered cell, population of cells, pharmaceutical composition, composition, or method of any one of embodiments 67-172, wherein the HLA-B guide RNA comprises SEQ ID NO: 89.

Embodiment 218 is the engineered cell, population of cells, pharmaceutical composition, composition, or method of any one of embodiments 67-172, wherein the HLA-B guide RNA comprises SEQ ID NO: 90.

Embodiment 219 is the engineered cell, population of cells, pharmaceutical composition, composition, or method of any one of embodiments 67-172, wherein the HLA-B guide RNA comprises SEQ ID NO: 91.

Embodiment 220 is the engineered cell, population of cells, pharmaceutical composition, composition, or method of any one of embodiments 67-172, wherein the HLA-B guide RNA comprises a guide sequence selected from SEQ ID NOs: 101, 103, 106, 107, 114, 117, 118, 125-129, 133, 137, 138, 141, 143, 144, 145, 159, 160, 163, 164, 165, 166, 169, 171, 172, 173, 176, 177, 178, 179, and 180.

Embodiment 221 is the engineered cell, population of cells, pharmaceutical composition, composition, or method of any one of embodiments 67-172, wherein the HLA-B guide RNA comprises a guide sequence selected from SEQ ID NOs: 101, 103, 106, 117, 118, 125-128, 133, 137-138, 141, 143-144, 159, 163, 164, 165, 166, 169, 171, 173, 177, 178, and 180.

Embodiment 222 is the engineered cell, population of cells, pharmaceutical composition, composition, or method of any one of embodiments 67-172, wherein the HLA-B guide RNA comprises a guide sequence selected from SEQ ID NOs: 101, 106, 114, 117-118, 125-128, 133, 137-138, 141, 143-144, 159, 163, 164, 165, 166, 169, 171, 173, 177, 178, and 180.

Embodiment 223 is the engineered cell, population of cells, pharmaceutical composition, composition, or method of any one of embodiments 67-181172, wherein the HLA-B guide RNA comprises a guide sequence selected from SEQ ID NOs: 101, 117-118, 125-128, 137-138, 144, 159, 163, 164, 165, 166, 169, 177, 178, and 180.

Embodiment 224 is the engineered cell, population of cells, pharmaceutical composition, composition, or method of any one of embodiments 67-172, wherein the HLA-B guide RNA comprises a guide sequence selected from SEQ ID NOs: 101, 117, 127, 137-138, 163, 164, 165, 166, 169, and 177.

Embodiment 225 is the engineered cell, population of cells, pharmaceutical composition, composition, or method of any one of embodiments 67-172, wherein the HLA-B guide RNA comprises a guide sequence selected from SEQ ID NOs: 101, 103, 106, 107, 117, 125-129, 137, 138, 141, 143, 144, 145, 159, 160, 163, 164, 165, 166, 169, 171, 172, 173, 176, 177, 178, 179, and 180.

Embodiment 226 is the engineered cell, population of cells, pharmaceutical composition, composition, or method of any one of embodiments 67-172, wherein the HLA-B guide RNA comprises a guide sequence comprising a sequence of any one of SEQ ID NOs: 163-166, 169, and 177.

Embodiment 227 is the engineered cell, population of cells, pharmaceutical composition, composition, or method of any one of embodiments 67-172, wherein the HLA-B guide RNA comprises SEQ ID NO: 101.

Embodiment 228 is the engineered cell, population of cells, pharmaceutical composition, composition, or method of any one of embodiments 67-172, wherein the HLA-B guide RNA comprises SEQ ID NO: 103.

Embodiment 229 is the engineered cell, population of cells, pharmaceutical composition, composition, or method of any one of embodiments 67-172, wherein the HLA-B guide RNA comprises SEQ ID NO: 106.

Embodiment 230 is the engineered cell, population of cells, pharmaceutical composition, composition, or method of any one of embodiments 67-172, wherein the HLA-B guide RNA comprises SEQ ID NO: 107.

Embodiment 231 is the engineered cell, population of cells, pharmaceutical composition, composition, or method of any one of embodiments 67-172, wherein the HLA-B guide RNA comprises SEQ ID NO: 117.

Embodiment 232 is the engineered cell, population of cells, pharmaceutical composition, composition, or method of any one of embodiments 67-172, wherein the HLA-B guide RNA comprises SEQ ID NO: 125.

Embodiment 233 is the engineered cell, population of cells, pharmaceutical composition, composition, or method of any one of embodiments 67-172, wherein the HLA-B guide RNA comprises SEQ ID NO: 126.

Embodiment 234 is the engineered cell, population of cells, pharmaceutical composition, composition, or method of any one of embodiments 67-172, wherein the HLA-B guide RNA comprises SEQ ID NO: 127.

Embodiment 235 is the engineered cell, population of cells, pharmaceutical composition, composition, or method of any one of embodiments 67-172, wherein the HLA-B guide RNA comprises SEQ ID NO: 128.

Embodiment 236 is the engineered cell, population of cells, pharmaceutical composition, composition, or method of any one of embodiments 67-172, wherein the HLA-B guide RNA comprises SEQ ID NO: 129.

Embodiment 237 is the engineered cell, population of cells, pharmaceutical composition, composition, or method of any one of embodiments 67-172, wherein the HLA-B guide RNA comprises SEQ ID NO: 137.

Embodiment 238 is the engineered cell, population of cells, pharmaceutical composition, composition, or method of any one of embodiments 67-172, wherein the HLA-B guide RNA comprises SEQ ID NO: 138.

Embodiment 239 is the engineered cell, population of cells, pharmaceutical composition, composition, or method of any one of embodiments 67-172, wherein the HLA-B guide RNA comprises SEQ ID NO: 141.

Embodiment 240 is the engineered cell, population of cells, pharmaceutical composition, composition, or method of any one of embodiments 67-172, wherein the HLA-B guide RNA comprises SEQ ID NO: 143.

Embodiment 241 is the engineered cell, population of cells, pharmaceutical composition, composition, or method of any one of embodiments 67-172, wherein the HLA-B guide RNA comprises SEQ ID NO: 144.

Embodiment 242 is the engineered cell, population of cells, pharmaceutical composition, composition, or method of any one of embodiments 67-172, wherein the HLA-B guide RNA comprises SEQ ID NO: 145.

Embodiment 243 is the engineered cell, population of cells, pharmaceutical composition, composition, or method of any one of embodiments 67-172, wherein the HLA-B guide RNA comprises SEQ ID NO: 159.

Embodiment 244 is the engineered cell, population of cells, pharmaceutical composition, composition, or method of any one of embodiments 67-172, wherein the HLA-B guide RNA comprises SEQ ID NO: 160.

Embodiment 245 is the engineered cell, population of cells, pharmaceutical composition, composition, or method of any one of embodiments 67-172, wherein the HLA-B guide RNA comprises SEQ ID NO: 163.

Embodiment 246 is the engineered cell, population of cells, pharmaceutical composition, composition, or method of any one of embodiments 67-172, wherein the HLA-B guide RNA comprises SEQ ID NO: 164.

Embodiment 247 is the engineered cell, population of cells, pharmaceutical composition, composition, or method of any one of embodiments 67-172, wherein the HLA-B guide RNA comprises SEQ ID NO: 165.

Embodiment 248 is the engineered cell, population of cells, pharmaceutical composition, composition, or method of any one of embodiments 67-172, wherein the HLA-B guide RNA comprises SEQ ID NO: 166.

Embodiment 249 is the engineered cell, population of cells, pharmaceutical composition, composition, or method of any one of embodiments 67-172, wherein the HLA-B guide RNA comprises SEQ ID NO: 169.

Embodiment 250 is the engineered cell, population of cells, pharmaceutical composition, composition, or method of any one of embodiments 67-172, wherein the HLA-B guide RNA comprises SEQ ID NO: 171.

Embodiment 251 is the engineered cell, population of cells, pharmaceutical composition, composition, or method of any one of embodiments 67-172, wherein the HLA-B guide RNA comprises SEQ ID NO: 172.

Embodiment 252 is the engineered cell, population of cells, pharmaceutical composition, composition, or method of any one of embodiments 67-172, wherein the HLA-B guide RNA comprises SEQ ID NO: 173.

Embodiment 253 is the engineered cell, population of cells, pharmaceutical composition, composition, or method of any one of embodiments 67-172, wherein the HLA-B guide RNA comprises SEQ ID NO: 176.

Embodiment 254 is the engineered cell, population of cells, pharmaceutical composition, composition, or method of any one of embodiments 67-172, wherein the HLA-B guide RNA comprises SEQ ID NO: 177.

Embodiment 255 is the engineered cell, population of cells, pharmaceutical composition, composition, or method of any one of embodiments 67-172, wherein the HLA-B guide RNA comprises SEQ ID NO: 178.

Embodiment 256 is the engineered cell, population of cells, pharmaceutical composition, composition, or method of any one of embodiments 67-172, wherein the HLA-B guide RNA comprises SEQ ID NO: 179.

Embodiment 257 is the engineered cell, population of cells, pharmaceutical composition, composition, or method of any one of embodiments 67-172, wherein the HLA-B guide RNA comprises SEQ ID NO: 180.

Embodiment 258 is the engineered cell, population of cells, pharmaceutical composition, composition, or method of any one of embodiments 67-172, wherein the HLA-B guide RNA comprises the sequence of any one of SEQ ID NOs: 2186-2191.

Embodiment 259 is the engineered cell, population of cells, pharmaceutical composition, composition, or method of any one of embodiments 67-172, wherein the HLA-B guide RNA comprises SEQ ID NO: 2186.

Embodiment 260 is the engineered cell, population of cells, pharmaceutical composition, composition, or method of any one of embodiments 67-172, wherein the HLA-B guide RNA comprises SEQ ID NO: 2187.

Embodiment 261 is the engineered cell, population of cells, pharmaceutical composition, composition, or method of any one of embodiments 67-172, wherein the HLA-B guide RNA comprises SEQ ID NO: 2188.

Embodiment 262 is the engineered cell, population of cells, pharmaceutical composition, composition, or method of any one of embodiments 67-172, wherein the HLA-B guide RNA comprises SEQ ID NO: 2189.

Embodiment 263 is the engineered cell, population of cells, pharmaceutical composition, composition, or method of any one of embodiments 67-172, wherein the HLA-B guide RNA comprises SEQ ID NO: 2190.

Embodiment 264 is the engineered cell, population of cells, pharmaceutical composition, composition, or method of any one of embodiments 67-172, wherein the HLA-B guide RNA comprises SEQ ID NO: 2191.

Embodiment 265 is the engineered cell, population of cells, pharmaceutical composition, or method of any one of embodiments 67-264, wherein the HLA-A guide RNA comprises SEQ ID NO: 313 or 314.

Embodiment 266 is the engineered cell, population of cells, pharmaceutical composition, or method of any one of embodiments 67-264, wherein the HLA-A guide RNA comprises SEQ ID NO: 314.

Embodiment 267 is the engineered cell, population of cells, pharmaceutical composition, or method of any one of embodiments 67-264, wherein the HLA-A guide RNA comprises SEQ ID NO: 315.

Embodiment 268 is the engineered cell, population of cells, pharmaceutical composition, or method of any one of embodiments 67-264, wherein the HLA-A guide RNA comprises SEQ ID NO: 316.

Embodiment 269 is the engineered cell, population of cells, pharmaceutical composition, or method of any one of embodiments 67-264, wherein the HLA-A guide RNA comprises SEQ ID NO: 317.

Embodiment 270 is the engineered cell, population of cells, pharmaceutical composition, or method of any one of embodiments 67-264, wherein the HLA-A guide RNA comprises SEQ ID NO: 318.

Embodiment 271 is the engineered cell, population of cells, pharmaceutical composition, or method of any one of embodiments 67-264, wherein the HLA-A guide RNA comprises SEQ ID NO: 326.

Embodiment 272 is the engineered cell, population of cells, pharmaceutical composition, or method of any one of embodiments 67-264, wherein the HLA-A guide RNA comprises SEQ ID NO: 337.

Embodiment 273 is the engineered cell, population of cells, pharmaceutical composition, or method of any one of embodiments 67-264, wherein the HLA-A guide RNA comprises SEQ ID NO: 338.

Embodiment 274 is the engineered cell, population of cells, pharmaceutical composition, or method of any one of embodiments 67-264, wherein the HLA-A guide RNA comprises SEQ ID NO: 339.

Embodiment 275 is the engineered cell, population of cells, pharmaceutical composition, or method of any one of embodiments 67-264, wherein the HLA-A guide RNA comprises SEQ ID NO: 341.

Embodiment 276 is the engineered cell, population of cells, pharmaceutical composition, or method of any one of embodiments 67-264, wherein the HLA-A guide RNA comprises SEQ ID NO: 343.

Embodiment 277 is the engineered cell, population of cells, pharmaceutical composition, or method of any one of embodiments 67-264, wherein the HLA-A guide RNA comprises SEQ ID NO: 345.

Embodiment 278 is the engineered cell, population of cells, pharmaceutical composition, or method of any one of embodiments 67-264, wherein the HLA-A guide RNA comprises SEQ ID NO: 362.

Embodiment 279 is the engineered cell, population of cells, pharmaceutical composition, or method of any one of embodiments 67-257 wherein the HLA-A guide RNA comprises SEQ ID NO: 576.

Embodiment 280 is the engineered cell, population of cells, pharmaceutical composition, or method of any one of embodiments 67-257 wherein the HLA-A guide RNA comprises SEQ ID NO: 571.

Embodiment 281 is the engineered cell, population of cells, pharmaceutical composition, composition, or method of any one of embodiments 1-280, for use to express a TCR with specificity for a polypeptide expressed by cancer cells.

Embodiment 282 is the engineered cell, population of cells, pharmaceutical composition, composition, or method of any one of embodiments 1-281, for use in administering to a subject as an adoptive cell transfer (ACT) therapy.

Embodiment 283 is the engineered cell, population of cells, pharmaceutical composition, composition, or method of any one of embodiments 1-282, for use in treating a subject with cancer.

Embodiment 284 is the engineered cell, population of cells, pharmaceutical composition, composition, or method of any one of embodiments 1-283, for use in treating a subject with an infectious disease.

Embodiment 285 is the engineered cell, population of cells, pharmaceutical composition, composition, or method of any one of embodiments 1-284, for use in treating a subject with an autoimmune disease.

Embodiment 286 is a cell bank comprising: (a) the engineered cells of any one of embodiments 1-56, 73-75, 94-285, or the engineered cells produced by the method of any one of embodiments 69-285; and (b) a catalogue comprising information documenting the HLA-A and HLA-C alleles of the donor cells in the cell bank.

Embodiment 287 is the cell bank of embodiment 286, wherein the cell bank comprises at least 3, 4, 5, 6, 7, 8, 9, 10, 11, 12, 13, 14, 15, 16, 17, 18, 19, 20, 25, 30, 35, or 40 donor cells that have a unique combination of HLA-A and HLA-C alleles as compared to other donor cells in the cell bank.

Embodiment 288 is a method of administering an engineered cell to a recipient subject in need thereof, the method comprising: (a) determining the HLA-A and HLA-C alleles of the recipient subject; (b) selecting an engineered cell or cell population of embodiments 1-56, 58, 60-63, 73-75, 94-285, or an engineered cell or cell population produced by the method of any one of embodiments 69-285, wherein the engineered cell comprises at least one of the same HLA-A or HLA-C alleles as the recipient subject; (c) administering the selected engineered cell to the recipient subject.

Embodiment 289 is the method of embodiment 288, wherein the subject has the HLA-A and HLA-C alleles of the engineered cell.

Embodiment 290 is the engineered cell, population, composition, pharmaceutical composition, or method of any one of embodiments 1-285, for use in administering to a partially matched subject for an adoptive cell transfer (ACT) therapy, wherein the partially matched subject has the HLA-A and HLA-C alleles of the engineered cell or cell population.

Embodiment 291 is the engineered cell, population, composition, pharmaceutical composition, or method of any one of embodiments 64-290, wherein the engineered cell or cell population comprises HLA-A and HLA-C alleles shared with the subject.

Embodiment 292 is the engineered cell, population, composition, pharmaceutical composition, or method of any one of embodiments 64-290, wherein the HLA-A and HLA-C alleles of the engineered cell or cell population consist of alleles that match one or more HLA-A and HLA-C alleles of the subject.

Embodiment 293 is the engineered cell, population, composition, pharmaceutical composition, or method of any one of the preceding embodiments 64-290, wherein the HLA-C alleles of the engineered cell or cell population consist of alleles that match one or both HLA-C alleles of the subject.

Embodiment 294 is a cell bank comprising: (a) the engineered cells of any one of embodiments 1-56, 73-75, 94-285, or the engineered cells produced by the method of any one of any one of embodiments 69-285; and (b) a catalogue comprising information documenting the HLA-C alleles of the donor cells in the cell bank.

Embodiment 295 is a method of administering an engineered cell to a recipient subject in need thereof, the method comprising: (a) determining the HLA-C alleles of the recipient subject; (b) selecting an engineered cell or cell population of any one of embodiments 1-56, 58, 60-63, 73-75, 94-285, or engineered cell or cell population produced by the method of any one of embodiments 69-285, wherein the engineered cell is homozygous for one of the HLA-C alleles of the recipient subject; (c) administering the selected engineered cell to the recipient subject.

Embodiment 296 is the method of embodiment 295, wherein the subject is homozygous or heterozygous for the HLA-C allele of the engineered cell.

Embodiment 297 is the engineered cell, population, composition, pharmaceutical composition, or method of any one of embodiments 1-285, for use in administering to a partially matched subject for an adoptive cell transfer (ACT) therapy, wherein the partially matched subject is homozygous or heterozygous for the HLA-C allele of the engineered cell or cell population.

Embodiment 298 is the engineered cell, population, composition, pharmaceutical composition, or method of any one of embodiments 64-297, wherein the engineered cell or cell population comprises HLA-C alleles shared with the subject.

Embodiment 299 is the engineered cell, population, composition, pharmaceutical composition, or method of any one of embodiments 64-298, wherein the HLA-C alleles of the engineered cell or cell population consist of alleles that match one or more HLA-C alleles of the subject.

Embodiment 300 is the engineered cell, population, composition, pharmaceutical composition, or method of any one of embodiments 64-299, wherein the HLA-C alleles of the engineered cell or cell population consist of alleles that match one or both HLA-C alleles of the subject.

### A. Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

An “allogeneic” cell, as used herein, refers to a cell originating from a donor subject of the same species as a recipient subject, wherein the donor subject and recipient subject have genetic dissimilarity, e.g., genes at one or more loci that are not identical. Thus, e.g., a cell is allogeneic with respect to the subject to be administered the cell. As used herein, a cell that is removed or isolated from a donor, that will not be re-introduced into the original donor, is considered an allogeneic cell.

An “autologous” cell, as used herein, refers to a cell derived from the same subject to whom the material will later be re-introduced. Thus, e.g., a cell is considered autologous if it is removed from a subject and it will then be re-introduced into the same subject.

“β2M” or “B2M,” as used herein, refers to nucleic acid sequence or protein sequence of “3-2 microglobulin”; the human gene has accession number NC_000015 (range 44711492..44718877), reference GRCh38.p13. The B2M protein is associated with MHC class I molecules as a heterodimer on the surface of nucleated cells and is required for MHC class I protein expression.

“CIITA” or “CIITA” or “C2TA,” as used herein, refers to the nucleic acid sequence or protein sequence of “class II major histocompatibility complex transactivator;” the human gene has accession number NC_000016.10 (range 10866208..10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532..29945870). The HLA-A gene is known to have thousands of different genotypic versions of the HLA-A gene across the population (and an individual may receive two different alleles of the HLA-A gene). A public database for HLA-A alleles, including sequence information, may be accessed at IPD-IMGT/HLA: www.ebi.ac.uk/ipd/imgt/hla/. All alleles of HLA-A are encompassed by the terms “HLA-A” and “HLA-A gene.”

The term “HLA-B,” as used herein in the context of HLA-B protein, refers to the MHC class I protein molecule, which is a heterodimer consisting of a heavy chain (encoded by the HLA-B gene) and a light chain (i.e., beta-2 microglobulin). “HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875..31357179). The HLA-B gene is known to have thousands of different genotypic versions of the HLA-B gene across the population (and an individual may receive two different alleles of the HLA-A gene). A public database for HLA-B alleles, including sequence information, may be accessed at IPD-IMGT/HLA: www.ebi.ac.uk/ipd/imgt/hla/. All alleles of HLA-B are encompassed by the terms “HLA-B” and “HLA-B gene.”

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749..31272092).

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

As used herein, the term “homozygous” refers to having two identical alleles of a particular gene.

As used herein, an HLA “allele” can refer to a named HLA-A, HLA-B, or HLA-C gene wherein the first four digits (or the first two sets of digits separated by a colon, e.g., HLA-A*02:101:01:02N where the first two sets of digits are bolded and in italics) of the name following “HLA-A”, HLA-B”, or “HLA-C” are specified. As known in the art, the first four digits (or first two sets of digits separated by a colon) specify the protein of the allele. For example, HLA-A*02:01 and HLA-A*01:02 are distinct HLA-A alleles. Further genotypes of each allele exist, such as, e.g., HLA-A*02:01:02:01. Further genotypes of a given allele are considered to be identical alleles, e.g., HLA-A*02:01:02:01 and HLA-A*02:01 are identical alleles. Thus, HLA alleles are homozygous when the alleles are identical (i.e., when the alleles have the same first four digits or same first two sets of digits separated by a colon).

“Matching” or “matched” refers to shared alleles between the donor and the recipient, e.g., identical alleles.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or NT-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to, for example, the guide that directs an RNA-guided DNA binding agent to a target DNA and can be a single guide RNA, or the combination of a crRNA and a trRNA (also known as tracrRNA). Exemplary gRNAs include Class II Cas nuclease guide RNAs, in modified or unmodified forms. The crRNA and trRNA may be associated as a single RNA molecule (single guide RNA, sgRNA) or in two separate RNA strands (dual guide RNA, dgRNA). “Guide RNA” or “gRNA” refers to each type. The trRNA may be a naturally occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 nucleotides in length, e.g., in the case of Streptococcus pyogenes (i.e., Spy Cas9 (SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more nucleotides. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Accordingly, in the case of Neisseria meningitides (i.e., Nine Cas9 (NmeCas9)) and related Cas9 homologs/orthologs, a guide sequence may be 19, 20, 21, preferably 22, 23, or 24 nucleotides in length, or may be 20-25 nucleotides in length. In some embodiments, the target sequence is in a gene or on a chromosome, for example, and is complementary to the guide sequence. In some embodiments, the degree of complementarity or identity between a guide sequence and its corresponding target sequence is at least 80%, 85%, preferably 90%, or 95%. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, preferably 23, or 24, nucleotides, or more. In some embodiments, the guide sequence and the target region may contain 1-2 mismatches where the guide sequence comprises at least 24 nucleotides, or more. In some embodiments, the guide sequence and the target region may contain 1-2 mismatches where the guide sequence comprises 24 nucleotides. That is, the guide sequence and the target region may form a duplex region having at least 2× base pairs, or more. In certain embodiments, the duplex region may include 1-2 mismatches such that guide strand and target sequence are not fully complementary. Mismatch positions are known in the art as provided in, for example, PAM distal mismatches tend to be better tolerated than PAM proximal matches. Mismatch tolerances at other positions are known in the art (see, e.g., Edraki et al., 2019. Mol. Cell, 73:1-13).

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the presence of a PAM and the sequence of the guide RNA. Exemplary RNA-guided DNA binding agents include Cas cleavases/nickases and inactivated forms thereof (“dCas DNA binding agents”). “Cas nuclease”, as used herein, encompasses Cas cleavases, Cas nickases, and dCas DNA binding agents. The dCas DNA binding agent may be a dead nuclease comprising non-functional nuclease domains (RuvC or HNH domain). In some embodiments the Cas cleavase or Cas nickase encompasses a dCas DNA binding agent modified to permit DNA cleavage, e.g. via fusion with a FokI domain. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases.

As used herein, a “Class 2 Cas nuclease” is a single-chain polypeptide with RNA-guided DNA binding activity. Class 2 Cas nucleases include Class 2 Cas cleavases/nickases (e.g., H840A or D10A variants of Spy Cas9 and D16A and H588A of Nine Cas9, e.g., Nme2 Cas9), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

Several Cas9 orthologs have been obtained from N. meningitidis (Esvelt et al., NAT. METHODS, vol. 10, 2013, 1116-1121; Hou et al., PNAS, vol. 110, 2013, pages 15644-15649) (Nme1Cas9, Nme2Cas9, and Nme3Cas9). The Nme2Cas9 ortholog functions efficiently in mammalian cells, recognizes an N4CC PAM, and can be used for in vivo editing with cognate gRNAs (Ran et al., NATURE, vol. 520, 2015, pages 186-191; Kim et al., NAT. COMMUN., vol. 8, 2017, pages 14500). Nme2Cas9 can be specific and selective, e.g. capable of low off-target editing (Lee et al., MOL. THER., vol. 24, 2016, pages 645-654; Kim et al., 2017). See also e.g., WO/2020081568 (e.g., pages 28 and 42), describing an Nme2Cas9 D16A nickase, the contents of which are hereby incorporated by reference in its entirety. Throughout, “NmeCas9” or “Nine Cas9” is generic and encompasses any type of NmeCas9, including, Nme1Cas9, Nme2Cas9, and Nme3Cas9.

As used herein, the term “editor” refers to an agent comprising a polypeptide that is capable of making a modification within a DNA sequence. In some embodiments, the editor is a cleavase, such as a Cas9 cleavase. In some embodiments, the editor is capable of deaminating a base within a DNA molecule, and it may be called a base editor. In some embodiments, the editor is capable of deaminating a cytosine (C) in DNA. In some embodiments, the editor is a fusion protein comprising an RNA-guided nickase fused to a cytidine deaminase. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an RNA-guided nickase fused to a cytidine deaminase and a UGI. In some embodiments, the editor lacks a UGI.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)).

As used herein, the term “APOBEC3” refers to a APOBEC3 protein, such as an APOBEC3 protein expressed by any of the seven genes (A3A-A3H) of the human APOBEC3 locus. The APOBEC3 may have catalytic DNA or RNA editing activity. An amino acid sequence of APOBEC3A has been described (UniPROT accession ID: p31941) and is included herein as SEQ ID NO: 799. In some embodiments, the APOBEC3 protein is a human APOBEC3 protein or a wild-type protein. Variants include proteins having a sequence that differs from wild-type APOBEC3 protein by one or several mutations (i.e. substitutions, deletions, insertions), such as one or several single point substitutions. For instance, a shortened APOBEC3 sequence could be used, e.g. by deleting several N-term or C-term amino acids, preferably one to four amino acids at the C-terminus of the sequence. As used herein, the term “variant” refers to allelic variants, splicing variants, and natural or artificial mutants, which are homologous to a APOBEC3 reference sequence. The variant is “functional” in that it shows a catalytic activity of DNA or RNA editing. In some embodiments, an APOBEC3 (such as a human APOBEC3A) has a wild-type amino acid position 57 (as numbered in the wild-type sequence). In some embodiments, an APOBEC3 (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided DNA nickase” means a polypeptide or complex of polypeptides having DNA nickase activity, wherein the DNA nickase activity is sequence-specific and depends on the sequence of the RNA. Exemplary RNA-guided DNA nickases include Cas nickases. Cas nickases include nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include variants in which only one of the two catalytic domains is inactivated, which have RNA-guided DNA nickase activity. Class 2 Cas nickases include polypeptides in which either the HNH or RuvC catalytic domain is inactivated, for example, Cas9 for example, Cas9 (e.g., H840A, D10A, or N863A variants of SpyCas9 or D16A variant of NmeCas9). Exemplary amino acid substitutions in the HNH or HNH-like nuclease domain or RuvC or RuvC-like domains for N. meningitidis include Nme2Cas9 D16A (HNH nickase) and Nme2Cas9 H588A (RuvC nickase), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence

(SEQ ID NO: 900)
SGSETPGTSESATPES,
(SEQ ID NO: 901)
SGSETPGTSESA,
or
(SEQ ID NO: 902)
SGSETPGTSESATPEGGSGGS.

As used herein, the term “uracil glycosylase inhibitor” or “UGI” refers to a protein that is capable of inhibiting a uracil-DNA glycosylase (UDG) base-excision repair enzyme.

As used herein, “open reading frame” or “ORF” of a gene refers to a sequence consisting of a series of codons that specify the amino acid sequence of the protein that the gene codes for. The ORF begins with a start codon (e.g., ATG in DNA or AUG in RNA) and ends with a stop codon, e.g., TAA, TAG or TGA in DNA or UAA, UAG, or UGA in RNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indel” refers to an insertion or deletion mutation consisting of a number of nucleotides that are either inserted, deleted, or inserted and deleted, e.g. at the site of double-stranded breaks (DSBs), in a target nucleic acid. As used herein, when indel formation results in an insertion, the insertion is a random insertion at the site of a DSB and is not generally directed by or based on a template sequence.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced” or “eliminated” surface expression relative to an unmodified cell as evidenced by a reduction in fluorescence signal upon staining with the same antibody against the protein. A cell that has “reduced” or “eliminated” surface expression of a protein by flow cytometry relative to an unmodified cell may be referred to as “negative” for expression of that protein as evidenced by a fluorescence signal similar to a cell stained with an isotype control antibody. The “reduction” or “elimination” of protein expression can be measured by other known techniques in the field with appropriate controls known to those skilled in the art.

As used herein, “knockdown” refers to a decrease in expression of a particular gene product (e.g., protein, mRNA, or both), e.g., as compared to expression of an unedited target sequence. Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include analyzing mRNA isolated from a sample of interest. In some embodiments, “knockdown” may refer to some loss of expression of a particular gene product, for example a decrease in the amount of mRNA transcribed or a decrease in the amount of protein expressed by a cell or population of cells (including in vivo populations such as those found in tissues).

As used herein, “knockout” (or “KO”) refers to a loss of expression from a particular gene or of a particular protein in a cell. Knockout can result in a decrease in expression below the level of detection of the assay. Knockout can be measured either by detecting total cellular amount of a protein in a cell, a tissue or a population of cells.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including recurrence of the symptom.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### B. Genetically Modified Cells

### 1. Engineered Human Cell Compositions

The present disclosure provides engineered human cell compositions which have reduced or eliminated surface expression of HLA-B protein relative to an unmodified cell, comprising a genetic modification in the HLA-B gene, wherein the cell is homozygous for HLA-A and homozygous for HLA-C. Additionally, the disclosure provides engineered human cell compositions which have reduced or eliminated surface expression of HLA-A and HLA-B protein relative to an unmodified cell, comprising (i) a genetic modification in the HLA-A gene and (ii) a genetic modification in the HLA-B gene, wherein the cell is homozygous for HLA-C. In some embodiments, the engineered human cell is an allogeneic cell. In some embodiments, the engineered human cell with reduced or eliminated HLA-B expression or HLA-A and HLA-B expression is useful for adoptive cell transfer therapies. In some embodiments, the engineered human cell comprises additional genetic modifications in the genome of the cell (e.g., reducing or elimination of MHC class II proteins, or reducing or eliminating endogenous T cell receptor (TCR) proteins, or introduction of an exogenous nucleic acid for expression) to yield a cell that is desirable for allogeneic transplant purposes.

In some embodiments, the engineered human cell is an allogeneic cell therapy. In some embodiments, the engineered human cell is transferred to a recipient that has the same HLA-A allele as the engineered human cell. In some embodiments, the engineered human cell is transferred to a recipient that has the same HLA-C allele as the engineered human cell. In some embodiments, the engineered human cell is transferred to a recipient that has the same HLA-A and HLA-C alleles as the engineered human cell. Thus, the engineered human cells disclosed herein provide a partial HLA match to a recipient, thereby reducing the risk of an adverse immune response.

In some embodiments, an engineered human cell is provided which has reduced or eliminated surface expression of HLA-B protein relative to an unmodified cell, comprising a genetic modification in the HLA-B gene, wherein the cell is homozygous for HLA-A and HLA-C.

In some embodiments, an engineered human cell is provided which has reduced or eliminated surface expression of HLA-A and HLA-B protein relative to an unmodified cell, comprising a genetic modification in the HLA-B gene, wherein the cell is homozygous for HLA-C.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from (a) chr6:31354480-31357174 or (b) chr6:31357084-31354647; wherein the cell is homozygous for HLA-A and HLA-C.

In some embodiments, an engineered human cell is provided which has reduced or eliminated surface expression of HLA-A and HLA-B protein relative to an unmodified cell, comprising a genetic modification in the HLA-A and HLA-B genes, (i) wherein the genetic modification in HLA-A comprises at least one nucleotide within the genomic coordinates chosen from chr6:29942854-chr6:29942913 and chr6:29943518-chr6:29943619; and (ii) wherein the genetic modification in HLA-B comprises at least one nucleotide within the genomic coordinates chosen from: (a) chr6:31354480-31357174 or (b) chr6: 31354623-31357108 or 31354497-31357157; and wherein the cell is homozygous for HLA-C.

In some embodiments, for each given range of genomic coordinates, a range may encompass +/−10 nucleotides on either end of the specified coordinates. For example, if chr6:29942854-chr6:29942913 is given, in some embodiments the genomic target sequence or genetic modification may fall within chr6:29942844-chr6:29942923. In some embodiments, for each given range of genomic coordinates, the range may encompass +/−5 nucleotides on either end of the range.

In some embodiments, a given range of genomic coordinates may comprise a target sequence on both strands of the DNA (i.e., the plus (+) strand and the minus (−) strand).

Genetic modifications in the HLA-A or HLA-B gene are described further herein. In some embodiments, a genetic modification in the HLA-A or HLA-B genes comprises any one or more of an insertion, deletion, substitution, or deamination of at least one nucleotide in a target sequence.

The engineered human cells described herein may comprise a genetic modification in any HLA-B allele of the HLA-B gene or a genetic modification in any HLA-A allele of the HLA-A gene. The HLA gene is located in chromosome 6 in a genomic region referred to as the HLA superlocus; hundreds of HLA-A and HLA-B alleles have been reported in the art (see e.g., Shiina et al., Journal of Human Genetics 54:15-39 (2009). Sequences for HLA-A and HLA-B alleles are available in the art (see e.g., IPD-IMGT/HLA database for retrieving sequences of specific HLA-A and HLA-B alleles https://www.ebi.ac.uk/ipd/imgt/hla/allele.html).

In some embodiments, the cell has reduced or eliminated expression of at least one HLA-A allele selected from: HLA-A1, HLA-A2, HLA-A3, HLA-AT1, and HLA-A24. In some embodiments, the cell has reduced or eliminated expression of HLA-A1. In some embodiments, the cell has reduced or eliminated expression of HLA-A2. In some embodiments, the cell has reduced or eliminated expression of HLA-A3. In some embodiments, the cell has reduced or eliminated expression of HLA-A11. In some embodiments, the cell has reduced or eliminated expression of HLA-A24.

In some embodiments, the cell has reduced or eliminated expression of at least one HLA-B allele selected from: HLA-B7, HLA-B8, HLA-B13, HLA-B21, HLA-B27, HLA-B35, HLA-B37, HLA-B38, HLA-B39, HLA-B40, HLA-B41, HLA-B42, HLA-B44, HLA-B45, HLA-B46, HLA-B47, HLA-B48, HLA-B49, HLA-B50, HLA-B51, HLA-B52, HLA-B56, HLA-B67, HLA-B73, HLA-B81, and HLA-B83. In some embodiments, the cell has reduced or eliminated expression of HLA-B7. In some embodiments, the cell has reduced or eliminated expression of HLA-B8. In some embodiments, the cell has reduced or eliminated expression of HLA-B13. In some embodiments, the cell has reduced or eliminated expression of HLA-B21. In some embodiments, the cell has reduced or eliminated expression of HLA-B27. In some embodiments, the cell has reduced or eliminated expression of HLA-B35. In some embodiments, the cell has reduced or eliminated expression of HLA-B37. In some embodiments, the cell has reduced or eliminated expression of HLA-B38. In some embodiments, the cell has reduced or eliminated expression of HLA-B39. In some embodiments, the cell has reduced or eliminated expression of HLA-B40. In some embodiments, the cell has reduced or eliminated expression of HLA-B41. In some embodiments, the cell has reduced or eliminated expression of HLA-B42. In some embodiments, the cell has reduced or eliminated expression of HLA-B44. In some embodiments, the cell has reduced or eliminated expression of HLA-B45. In some embodiments, the cell has reduced or eliminated expression of HLA-B46. In some embodiments, the cell has reduced or eliminated expression of HLA-B47. In some embodiments, the cell has reduced or eliminated expression of HLA-B48. In some embodiments, the cell has reduced or eliminated expression of HLA-B49. In some embodiments, the cell has reduced or eliminated expression of HLA-B50. In some embodiments, the cell has reduced or eliminated expression of HLA-B51. In some embodiments, the cell has reduced or eliminated expression of HLA-B52. In some embodiments, the cell has reduced or eliminated expression of HLA-B56. In some embodiments, the cell has reduced or eliminated expression of HLA-B57. In some embodiments, the cell has reduced or eliminated expression of HLA-B67. In some embodiments, the cell has reduced or eliminated expression of HLA-B73. In some embodiments, the cell has reduced or eliminated expression of HLA-B81. In some embodiments, the cell has reduced or eliminated expression of HLA-B83.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355182-31355202; chr6:31355348-31355368; chr6:31355145-31355165; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355347-31355367; chr6:31355432-31355452; chr6:31355340-31355360; chr6:31355410-31355430; chr6:31355414-31355434; or chr6:31355409-31355429.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355182-31355202; chr6:31355349-31355369; chr6:31355348-31355368; or chr6:31355145-31355165.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355348-31355368; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; or chr6:31355414-31355434.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355348-31355368; chr6:31355349-31355369; chr6:31356381-31356401; chr6:31356380-31356400; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; chr6:31355410-31355430; chr6:31355414-31355434; or chr6:31355409-31355429.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355348-31355368, chr6:31355349-31355369, chr6:31355192-31355212, chr6:31355347-31355367, chr6:31355340-31355360, chr6:31355409-31355429.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355349-31355369 or chr6:31355348-31355368.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355192-31355212 or chr6:31355347-31355367.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355347-31355367; chr6:31355340-31355360; or chr6:31355409-31355429.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355348-31355368; chr6:31355145-31355165; chr6:31355347-31355367; chr6:31355432-31355452; or chr6:31355340-31355360.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; and chr6:31355469-31355493.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355361-31355385; chr6:31355379-31355403; chr6:31355491-31355515; chr6:31355356-31355380; chr6:31355366-31355390; chr6:31355417-31355441; chr6:31357078-31357102; chr6:31355460-31355484; chr6:31355415-31355439; chr6:31355166-31355190; chr6:31355378-31355402; chr6:31355401-31355425; chr6:31356262-31356286; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; or chr6:31356426-31356450.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355379-31355403; chr6:31355491-31355515; chr6:31355361-31355385; chr6:31355221-31355245; chr6:31355222-31355246; chr6:31355205-31355229; chr6:31355446-31355470; chr6:31356425-31356449; or chr6:31355441-31355465.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31355221-31355245; chr6:31355222-31355246; chr6:31355205-31355229; chr6:31355446-31355470; chr6:31356425-31356449; or chr6:31355441-31355465.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; chr6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from: (a) chr6:31355348-31355368; or (b) chr6:31355390-31355414; chr6:31355417-31355441; or chr6: 31356386-31356410.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: (a) chr6:31355145-31356401 or (b) chr6:31357084-31354647. In some embodiments, the cell is homozygous for HLA-A and homozygous for HLA-C.

In some embodiments, an engineered human cell is provided which has reduced or eliminated surface expression of HLA-A and HLA-B protein relative to an unmodified cell, comprising a genetic modification in an HLA-A and HLA-B gene, wherein the genetic modification in HLA-A comprises an indel, a C to T substitution, or an A to G substitution within the genomic coordinates chosen from chr6:29942854-chr6:29942913 and chr6:29943518-chr6:29943619; and wherein the genetic modification in HLA-B comprises an indel, a C to T substitution, or an A to G substitution within the genomic coordinates chosen from: (a) chr6:31355145-31356401 or (b) chr6:31357084-31354647. In some embodiments, the cell is homozygous for HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355182-31355202; chr6:31355348-31355368; chr6:31355145-31355165; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355347-31355367; chr6:31355432-31355452; chr6:31355340-31355360; chr6:31355410-31355430; chr6:31355414-31355434; or chr6:31355409-31355429. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355182-31355202; chr6:31355349-31355369; chr6:31355348-31355368; or chr6:31355145-31355165. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355348-31355368; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; or chr6:31355414-31355434. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355348-31355368; chr6:31355349-31355369; chr6:31356381-31356401; chr6:31356380-31356400; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; chr6:31355410-31355430; chr6:31355414-31355434; or chr6:31355409-31355429. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355348-31355368, chr6:31355349-31355369, chr6:31355192-31355212, chr6:31355347-31355367, chr6:31355340-31355360, chr6:31355409-31355429. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355349-31355369 or chr6:31355348-31355368. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: chr6:31355192-31355212 or chr6:31355347-31355367. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355347-31355367; chr6:31355340-31355360; or chr6:31355409-31355429. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355348-31355368; chr6:31355145-31355165; chr6:31355347-31355367; chr6:31355432-31355452; or chr6:31355340-31355360. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; chr6:31355469-31355493; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: chr6:31356777-31356801; chr6:31355492-31355516; chr6:31355361-31355385; chr6:31355379-31355403; chr6:31355491-31355515; chr6:31355356-31355380; chr6:31355366-31355390; chr6:31355417-31355441; chr6:31357078-31357102; chr6:31355460-31355484; chr6:31355415-31355439; chr6:31355166-31355190; chr6:31355378-31355402; chr6:31355401-31355425; chr6:31356262-31356286; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; or chr6:31356426-31356450. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31356777-31356801; chr6:31355492-31355516; chr6:31355379-31355403; chr6:31355491-31355515; chr6:31355361-31355385; chr6:31355221-31355245; chr6:31355222-31355246; chr6:31355205-31355229; chr6:31355446-31355470; chr6:31356425-31356449; or chr6:31355441-31355465. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from; chr6:31355221-31355245; chr6:31355222-31355246; chr6:31355205-31355229; chr6:31355446-31355470; chr6:31356425-31356449; or chr6:31355441-31355465. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: (a) chr6:31355348-31355368; or (b) chr6:31355390-31355414; chr6:31355417-31355441; or chr6: 31356386-31356410. In some embodiments, the cell is homozygous for HLA-A and HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791, wherein the genetic modification comprises at least 2, at least 3, at least 4, at least 5, at least 6, at least 7, at least 8, at least 9, or at least 10 contiguous nucleotides within the genomic coordinates. In some embodiments, the cell is homozygous for HLA-A. In some embodiments, the cell is homozygous for HLA-C. In some embodiments, the cell is homozygous for HLA-A and homozygous for HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791; wherein the genetic modification comprises at least 5 contiguous nucleotides within the genomic coordinates. In some embodiments, the cell is homozygous for HLA-A. In some embodiments, the cell is homozygous for HLA-C. In some embodiments, the cell is homozygous for HLA-A and homozygous for HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791, wherein the genetic modification comprises at least 6, 7, 8, 9, or 10 contiguous nucleotides within the genomic coordinates. In some embodiments, the genetic modification comprises at least 6 contiguous nucleotides within the genomic coordinates. In some embodiments, the genetic modification comprises at least 7 contiguous nucleotides within the genomic coordinates. In some embodiments, the genetic modification comprises at least 8 contiguous nucleotides within the genomic coordinates. In some embodiments, the genetic modification comprises at least 9 contiguous nucleotides within the genomic coordinates. In some embodiments, the genetic modification comprises at least 10 contiguous nucleotides within the genomic coordinates. In some embodiments, the cell is homozygous for HLA-A. In some embodiments, the cell is homozygous for HLA-C. In some embodiments, the cell is homozygous for HLA-A and homozygous for HLA-C.

In some embodiments, an engineered human cell is provided which has reduced or eliminated surface expression of HLA-B protein relative to an unmodified cell, comprising a genetic modification in an HLA-B gene, wherein the genetic modification comprises an indel, a C to T substitution, or an A to G substitution within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791; wherein the genetic modification comprises at least one C to T substitution or at least one A to G substitution within the genomic coordinates. In some embodiments, the cell is homozygous for HLA-A. In some embodiments, the cell is homozygous for HLA-C. In some embodiments, the cell is homozygous for HLA-A and homozygous for HLA-C.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; and chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791. Due to allelic polymorphism, in some embodiments, the target sequences may comprise 1, 2, or 3 mismatches from the genomic sequence of hg38. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates. In some embodiments, the gene editing system comprises an RNA-guided DNA binding agent, such as an S. pyogenes Cas9, an N. meningitidis Cas9, or a base editor that comprises an S. pyogenes or N. meningitidis Cas9 nickase.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates. In some embodiments, the gene editing system comprises an RNA-guided DNA binding agent, such as an S. pyogenes Cas9.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; chr6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates. In some embodiments, the gene editing system comprises an RNA-guided DNA binding agent, such as an N. meningitidis Cas9 or Nme2Cas9.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates. In some embodiments, the gene editing system comprises an RNA-guided DNA binding agent, such as a base editor comprising a deaminase and an S. pyogenes Cas9 nickase.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; chr6:31355409-31355429; or (b) chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chosen from: (a) chr6:31355145-31356401 or (b) chr6:31357084-31354647. In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; and chr6:31355409-31355429.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355182-31355202.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355348-31355368.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355180-31355200.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355145-31355165.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355349-31355369.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355157-31355177.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356381-31356401.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356380-31356400.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355204-31355224.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355205-31355225.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355185-31355205.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355191-31355211.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355192-31355212.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355190-31355210.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355193-31355213.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355198-31355218.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355320-31355340.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355319-31355339.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355178-31355198.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355347-31355367.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355432-31355452.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355340-31355360.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355576-31355596.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355410-31355430.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355419-31355439.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355414-31355434.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355409-31355429.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356777-31356801.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355492-31355516.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355491-31355515.

In some embodiments, an engineered human cell is provided wherein the HLA-B expression is reduced or eliminated by a gene editing system that binds to an HLA-B genomic target sequence comprising at least 5 contiguous nucleotides within the genomic coordinates 31355469-31355493.

In some embodiments, an engineered human cell is provided wherein the HLA-B expression is reduced or eliminated by a gene editing system that binds to an HLA-B genomic target sequence comprising at least 5 contiguous nucleotides within the genomic coordinates 31355460-31355484.

In some embodiments, an engineered human cell is provided wherein the HLA-B expression is reduced or eliminated by a gene editing system that binds to an HLA-B genomic target sequence comprising at least 5 contiguous nucleotides within the genomic coordinates 31355419-31355443.

In some embodiments, an engineered human cell is provided wherein the HLA-B expression is reduced or eliminated by a gene editing system that binds to an HLA-B genomic target sequence comprising at least 5 contiguous nucleotides within the genomic coordinates 31355415-31355439.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355417-31355441.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355401-31355425.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355390-31355414.

In some embodiments, an engineered human cell is provided wherein the HLA-B expression is reduced or eliminated by a gene editing system that binds to an HLA-B genomic target sequence comprising at least 5 contiguous nucleotides within the genomic coordinates 31355379-31355403.

In some embodiments, an engineered human cell is provided wherein the HLA-B expression is reduced or eliminated by a gene editing system that binds to an HLA-B genomic target sequence comprising at least 5 contiguous nucleotides within the genomic coordinates 31355378-31355402.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355369-31355393.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr 6:31355361-31355385.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr 6:31355366-31355390.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr 6:31355356-31355380.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355221-31355245.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355222-31355246.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355205-31355229.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355446-31355470.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356425-31356449.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355441-31355465.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355203-31355227.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356437-31356461.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356426-31356450.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356763-31356787.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356764-31356788.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356762-31356786.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31355204-31355228.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356436-31356460.

In some embodiments, an engineered human cell is provided wherein the HLA-B expression is reduced or eliminated by a gene editing system that binds to an HLA-B genomic target sequence comprising at least 5 contiguous nucleotides within the genomic coordinates chr6:31356767-31356791.

In some embodiments, the HLA-B genomic target sequence comprises at least 10 contiguous nucleotides within the genomic coordinates. In some embodiments, the HLA-B genomic target sequence comprises at least 15 contiguous nucleotides within the genomic coordinates.

In some embodiments, the HLA-B genomic target sequence comprises at least 17, 18, 19 20, 21, 22, 23, or 24 contiguous nucleotides within the genomic coordinates.

In some embodiments, the gene editing system comprises a transcription activator-like effector nuclease (TALEN). In some embodiments, the gene editing system comprises a zinc finger nuclease. In some embodiments, the gene editing system comprises a CRISPR/Cas system, such as a class 2 system. In some embodiments, the gene editing system comprises an RNA-guided DNA-binding agent or a nucleic acid encoding an RNA-guided DNA binding agent.

Exemplary RNA-guided DNA binding agents are shown in Table 1A below.

TABLE 1A
Exemplary RNA-guided DNA binding agents.

| RNA-guided DNA binding agent | PAM | Guide Length |
|---|---|---|
| Cas9 nuclease from S. pyogenes | NGG | 20 bp |
| Cas9 nuclease from Neisseria | NNNNG[A/C]TT | 20 bp |
| meningitidis | | |
| Cas9 nuclease from Streptococcus | NNAGAAW | 20 bp |
| thermophilus | | |
| Cas9 | NNG(A/G)(A/G)T | 20 bp |
| nuclease is from Staphylococcus | | |
| aureus | | |
| Cpf1 nuclease | TTTN | 23 bp |
| from Francisella novicida | | |
| Cpf1 nuclease | TTTV | 23 bp |
| from Acidaminococcus sp. | | |
| Cpf1 nuclease | TTTV | 23 bp |
| from Lachnospiraceae bacterium | | |
| C-to-T base editor* | NGG | 20 bp |
| A-to-G base editor* | NGG | 20 bp |
| Cas12a | same as Cpf1 | |
| CasX | TTCN | 20 bp |
| NME2 | NNNNCC | 24 bp |

*Exemplary base editor based on deaminase-SpyCas9 nickase. As is apparent, the base editor specificity, including PAM, will vary with its nickase.

In some embodiments, the RNA-guided DNA-binding agent or nucleic acid encoding the RNA-guided DNA binding agent comprises a Cas9 protein. In some embodiments, the RNA-guided DNA binding agent is selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, Mad7 nuclease, ARCUS nucleases, and CasX. In some embodiments, the RNA-guided DNA binding agent comprises a polypeptide selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX.

In some embodiments, the RNA-guided DNA-binding agent or nucleic acid encoding the RNA-guided DNA binding agent is S. pyogenes Cas9. In some embodiments, the RNA-guided DNA-binding agent or nucleic acid encoding the RNA-guided DNA binding agent is N. meningitidis Cas9, e.g. Nme2Cas9. In some embodiments, the RNA-guided DNA-binding agent or nucleic acid encoding the RNA-guided DNA binding agent is S. thermophilus Cas9. In some embodiments, the RNA-guided DNA-binding agent or nucleic acid encoding the RNA-guided DNA binding agent is S. aureus Cas9. In some embodiments, the RNA-guided DNA-binding agent or nucleic acid encoding the RNA-guided DNA binding agent is Cpf1 from F. novicida. In some embodiments, the RNA-guided DNA-binding agent or nucleic acid encoding the RNA-guided DNA binding agent is Cpf1 from Acidaminococcus sp. In some embodiments, the RNA-guided DNA-binding agent or nucleic acid encoding the RNA-guided DNA binding agent is Cpf1 from Lachnospiraceae bacterium ND2006. In some embodiments, the RNA-guided DNA-binding agent or nucleic acid encoding the RNA-guided DNA binding agent is a C to T base editor. In some embodiments, the RNA-guided DNA-binding agent or nucleic acid encoding the RNA-guided DNA binding agent is a A to G base editor. In some embodiments, the base editor comprises a deaminase and an RNA-guided nickase. In some embodiments, the RNA-guided DNA-binding agent or nucleic acid encoding the RNA-guided DNA binding agent comprises a APOBEC3A deaminase (A3A) and an RNA-guided nickase. In some embodiments, the RNA-guided nickase is a SpyCas9 nickase. In some embodiments, the RNA-guided nickase comprises an NmeCas9 nickase. In some embodiments, the RNA-guided DNA-binding agent or nucleic acid encoding the RNA-guided DNA binding agent is Cas12a. In some embodiments, the RNA-guided DNA-binding agent or nucleic acid encoding the RNA-guided DNA binding agent is CasX.

In any of the above embodiments, the C comprises an RNA-guided DNA binding agent, or a nucleic acid encoding an RNA-guided DNA binding agent. In some embodiments, the RNA-guided DNA binding agent comprises a Cas9. In some embodiments, the RNA-guided DNA binding agent is an S. pyogenes Cas9. In some embodiments, the RNA-guided DNA binding agent is a base editor. In some embodiments the base editor comprises a C to T deaminase and an RNA-guided nickase such as an S. pyogenes Cas9 nickase. In some embodiments the base editor comprises a A to G deaminase and an RNA-guided nickase such as an S. pyogenes Cas9 nickase.

In any of the above embodiments, the gene editing system comprises an RNA-guided DNA binding agent, or a nucleic acid encoding an RNA-guided DNA binding agent. In some embodiments, the RNA-guided DNA binding agent comprises a Cas9. In some embodiments, the RNA-guided DNA binding agent is an N. meningitidis or Nme2 Cas9. In some embodiments, the RNA-guided DNA binding agent is a base editor. In some embodiments the base editor comprises a C to T deaminase and an RNA-guided nickase such as an N. meningitidis or Nme2 Cas9 nickase. In some embodiments the base editor comprises a A to G deaminase and an RNA-guided nickase such as an N. meningitidis or Nme2 Cas9 nickase.

In some embodiments, the gene editing system further comprises a uracil glycosylase inhibitor (UGI), and the UGI and the base editor are comprised in a single polypeptide. In some embodiments, the gene editing system comprises a UGI, and the UGI and the base editor are comprised in different polypeptides. In some embodiments, the base editor comprises a cytidine deaminase and an RNA-guided nickase. In some embodiments, the cytidine deaminase, the RNA-guided nickase, and the UGI are comprised in a single polypeptide. In some embodiments, the cytidine deaminase, the RNA-guided nickase, and the UGI are comprised in different polypeptides. In some embodiments, the cytidine deaminase and the RNA-guided nickase are comprised in a single polypeptide, and wherein the UGI is comprised in a different polypeptide.

In some embodiments, when the engineered cell is homozygous for HLA-A, the HLA-A allele is selected from any one of the following HLA-A alleles: HLA-A*02:01; HLA-A*01:01; HLA-A*03:01; HLA-A*11:01; HLA-A*26:01; HLA-A*68:01; HLA-A*29:02; HLA-A*31:01; HLA-A*32:01; HLA-A*30:02; HLA-A*25:01; HLA-A*33:01; HLA-A*02:02; HLA-A*74:01; HLA-A*02:02; HLA-A*29:01; HLA-A*02:03; HLA-A*02:05; HLA-A*24:07; HLA-A*11:02; HLA-A*36:01; HLA-A*02:22; HLA-A*34:02; HLA-A*01:03; HLA-A*24:02; HLA-A*02:07; HLA-A*23:01; HLA-A*30:01; HLA-A*33:03; HLA-A*02:06; HLA-A*34:02; and HLA-A*68:02.

In some embodiments, when the engineered cell is homozygous for HLA-C, the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*08:01; HLA-C*03:02; HLA-C*16:01; HLA-C*15:02; HLA-C*03:04; HLA-C*12:03; HLA-C*02:10; HLA-C*05:01; HLA-C*12:02; HLA-C*14:02; HLA-C*04:01; HLA-C*03:03; HLA-C*07:04; HLA-C*17:01; HLA-C*01:02; and HLA-C*02:02.

In some embodiments, when the engineered cell is homozygous for HLA-C, the HLA-C allele is HLA-C*03:04. In some embodiments, when the engineered cell is homozygous for HLA-C, the HLA-C allele is HLA-C*06:02. In some embodiments, when the engineered cell is homozygous for HLA-C, the HLA-C allele is HLA-C*01:02. In some embodiments, when the engineered cell is homozygous for HLA-C, the HLA-C allele is HLA-C*08:01. In some embodiments, when the engineered cell is homozygous for HLA-C, the HLA-C allele is HLA-C*03:02.

In some embodiments, the engineered cell is homozygous for HLA-A and homozygous for HLA-C, the HLA-A and HLA-C allele pair is selected from the following: HLA-A*01:01 and HLA-C*07:01; HLA-A*02:01 and HLA-C*07:02; HLA-A*02:01 and HLA-C*05:01; HLA-A*03:01 and HLA-C*07:02; HLA-A*02:01 and HLA-C*04:01; HLA-A*02:01 and HLA-C*03:04; HLA-A*01:01 and HLA-C*06:02; HLA-A*03:01 and HLA-C*04:01; HLA-A*02:01 and HLA-C*07:01; HLA-A*24:02 and HLA-C*04:01; HLA-A*29:02 and HLA-C*16:01; HLA-A*02:01 and HLA-C*06:02; HLA-A*24:02 and HLA-C*07:02; HLA-A*26:01 and HLA-C*12:03; HLA-A*11:01 and HLA-C*04:01; HLA-A*25:01 and HLA-C*12:03; HLA-A*02:01 and HLA-C*02:02; HLA-A*24:02 and HLA-C*03:03; HLA-A*30:01 and HLA-C*06:02; HLA-A*02:01 and HLA-C*01:02; HLA-A*11:01 and HLA-C*07:02; HLA-A*03:01 and HLA-C*07:01; HLA-A*23:01 and HLA-C*04:01; HLA-A*24:02 and HLA-C*07:01; HLA-A*31:01 and HLA-C*03:04; HLA-A*33:01 and HLA-C*08:02; HLA-A*02:01 and HLA-C*03:03; HLA-A*11:01 and HLA-C*01:02; HLA-A*01:01 and HLA-C*04:01; HLA-A*03:01 and HLA-C*06:02.

The HLA-A and HLA-C allele pairs disclosed herein cumulatively cover about 810% of the population. The cumulative frequency of HLA-A and HLA-C allele pairs is shown in Table 1B below.

TABLE 1B
Cumulative Frequency of HLA-A and
HLA-C Alleles in the Population.

| Cumulative Frequency | Alleles |
|---|---|
| 0.136 | HLA-A*01:01 and HLA- C*07:01 |
| 0.231 | HLA-A*02:01 and HLA-C*07:02 |
| 0.299 | HLA-A*02:01 and HLA-C*05:01 |
| 0.361 | HLA-A*03:01 and HLA-C*07:02 |
| 0.417 | HLA-A*02:01 and HLA-C*04:01 |
| 0.460 | HLA-A*02:01 and HLA-C*03:04 |
| 0.493 | HLA-A*01:01 and HLA-C*06:02 |
| 0.524 | HLA-A*03:01 and HLA-C*04:01 |
| 0.554 | HLA-A*02:01 and HLA-C*07:01 |
| 0.579 | HLA-A*24:02 and HLA-C*04:01 |
| 0.600 | HLA-A*29:02 and HLA-C*16:01 |
| 0.621 | HLA-A*02:01 and HLA-C*06:02 |
| 0.640 | HLA-A*24:02 and HLA-C*07:02 |
| 0.657 | HLA-A*26:01 and HLA-C*12:03 |
| 0.673 | HLA-A*11:01 and HLA-C*04:01 |
| 0.686 | HLA-A*25:01 and HLA-C*12:03 |
| 0.698 | HLA-A*02:01 and HLA-C*02:02 |
| 0.710 | HLA-A*24:02 and HLA-C*03:03 |
| 0.720 | HLA-A*30:01 and HLA-C*06:02 |
| 0.730 | HLA-A*02:01 and HLA-C*01:02 |
| 0.740 | HLA-A*11:01 and HLA-C*07:02 |
| 0.749 | HLA-A*03:01 and HLA-C*07:01 |
| 0.758 | HLA-A*23:01 and HLA-C*04:01 |
| 0.766 | HLA-A*24:02 and HLA-C*07:01 |
| 0.773 | HLA-A*31:01 and HLA-C*03:04 |
| 0.780 | HLA-A*33:01 and HLA-C*08:02 |
| 0.787 | HLA-A*02:01 and HLA-C*03:03 |
| 0.794 | HLA-A*11:01 and HLA-C*01:02 |
| 0.800 | HLA-A*01:01 and HLA-C*04:01 |
| 0.806 | HLA-A*03:01 and HLA-C*06:02 |

In some embodiments, an engineered human cell which has reduced or eliminated surface expression of HLA-B protein relative to an unmodified cell is provided, that is homozygous for HLA-A and homozygous for HLA-C, further has reduced or eliminated surface expression of MHC class II protein. In some embodiments, the engineered human cell has a genetic modification in a gene that reduces or eliminates surface expression of MHC class II protein. In some embodiments, the engineered human cell has a genetic modification in the CIITA gene. In some embodiments, the engineered human cell has a genetic modification in the HLA-DR gene. In some embodiments, the engineered human cell has a genetic modification in the HLA-DQ gene. In some embodiments, the engineered human cell has a genetic modification in the HLA-DP gene. In some embodiments, the engineered human cell has a genetic modification in the RFX gene. In some embodiments, the engineered human cell has a genetic modification in the CREB gene. In some embodiments, the engineered human cell has a genetic modification in the Nuclear Factor (NF)-gamma gene.

In some embodiments, an engineered human cell which has reduced or eliminated surface expression of HLA-A and HLA-B protein relative to an unmodified cell is provided, that is homozygous for HLA-C, further has reduced or eliminated surface expression of MHC class II protein. In some embodiments, the engineered human cell has a genetic modification in a gene that reduces or eliminates surface expression of MHC class II protein. In some embodiments, the engineered human cell has a genetic modification in the CIITA gene. In some embodiments, the engineered human cell has a genetic modification in the HLA-DR gene. In some embodiments, the engineered human cell has a genetic modification in the HLA-DQ gene. In some embodiments, the engineered human cell has a genetic modification in the HLA-DP gene. In some embodiments, the engineered human cell has a genetic modification in the RFX gene. In some embodiments, the engineered human cell has a genetic modification in the CREB gene. In some embodiments, the engineered human cell has a genetic modification in the Nuclear Factor (NF)-gamma gene.

In some embodiments, an engineered human cell which has reduced or eliminated surface expression of HLA-B protein relative to an unmodified cell is provided, that is homozygous for HLA-A and homozygous for HLA-C, further has reduced or eliminated surface expression of TRAC protein. In some embodiments, an engineered human cell which has reduced or eliminated surface expression of HLA-B protein relative to an unmodified cell is provided, that is homozygous for HLA-A and HLA-C, further has reduced or eliminated surface expression of TRBC protein.

In some embodiments, an engineered human cell which has reduced or eliminated surface expression of HLA-B protein relative to an unmodified cell is provided, that is homozygous for HLA-A and homozygous for HLA-C, further has reduced or eliminated surface expression of TRAC protein. In some embodiments, an engineered human cell which has reduced or eliminated surface expression of HLA-A and HLA-B protein relative to an unmodified cell is provided, that is homozygous for HLA-C, further has reduced or eliminated surface expression of TRBC protein.

In some embodiments, an engineered human cell which has reduced or eliminated surface expression of HLA-A and HLA-B protein relative to an unmodified cell is provided, that is homozygous for HLA-C, further has reduced or eliminated surface expression of TRAC protein. In some embodiments, an engineered human cell which has reduced or eliminated surface expression of HLA-A and HLA-B protein relative to an unmodified cell is provided, that is homozygous for HLA-C, further has reduced or eliminated surface expression of TRBC protein.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from (a) chr6:31355145-31356401 or (b) chr6: 31357084-31354647, and wherein the engineered cell further comprises a genetic modification in a gene that reduces or eliminates the surface expression of MHC class II protein. In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from (a) chr6:31355182-31355596 or (b) chr6: 31355203-31356461, and wherein the engineered cell further comprises a genetic modification in the CIITA gene.

In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from (a) chr6:31355182-31355596 or (b) chr6: 31355203-31356461, and wherein the engineered cell further comprises a genetic modification in the TRAC gene. In some embodiments, an engineered human cell is provided which has reduced or eliminated surface expression of HLA-B protein relative to an unmodified cell, comprising a genetic modification in the HLA-B gene, wherein the genetic modification comprises at least one nucleotide within the genomic coordinates chosen from (a) chr6: 31355182-31355596 or (b) chr6: chr6:31355203-31356461, and wherein the engineered cell further comprises a genetic modification in the TRBC gene.

In some embodiments, an engineered human cell is provided which has reduced or eliminated surface expression of HLA-A B protein relative to an unmodified cell, comprising a genetic modification in the HLA-B gene, wherein the genetic modification comprises at least one nucleotide within the genomic coordinates chosen from (a) chr6:31354480-31357174; chr631355145-31356401 or (b) chr6: chr6:31355203-31356461, and wherein the engineered cell further comprises an exogenous nucleic acid. In some embodiments, the engineered cell comprises an exogenous nucleic acid encoding a targeting receptor that is expressed on the surface of the engineered cell. In some embodiments, the targeting receptor is a CAR or a universal CAR. In some embodiments, the targeting receptor is a TCR. In some embodiments, the targeting receptor is a WT1 TCR. In some embodiments, the targeting receptor is a ligand for the receptor. In some embodiments, the targeting receptor is a hybrid CAR/TCR. In some embodiments, the targeting receptor comprises an antigen recognition domain (e.g., a cancer antigen recognition domain) and a subunit of a TCR. In some embodiments, the targeting receptor is a cytokine receptor. In some embodiments, the targeting receptor is a chemokine receptor. In some embodiments, the targeting receptor is a B cell receptor (BCR). In some embodiments, the engineered cell further comprises an exogenous nucleic acid encoding a polypeptide that is secreted by the engineered cell (i.e., a soluble polypeptide). In some embodiments, the exogenous nucleic acid encodes a therapeutic polypeptide. In some embodiments, the secreted polypeptide is an antibody. In some embodiments, the secreted polypeptide is an enzyme. In some embodiments, the exogenous nucleic acid encodes an antibody encodes a cytokine. In some embodiments, the exogenous nucleic acid encodes a chemokine. In some embodiments, the exogenous nucleic acid encodes a fusion protein.

The engineered human cell may be any of the exemplary cell types disclosed herein. Further, because MHC class I molecules are expressed on all nucleated cells, the engineered human cell may be any nucleated cell. In some embodiments, the engineered cell is an immune cell. In some embodiments, the engineered cell is a stem cell such as a hematopoietic stem cell (HSC). In some embodiments, the engineered cell is an induced pluripotent stem cell (iPSC). In some embodiments, the engineered cell is a mesenchymal stem cell (MSC). In some embodiments, the engineered cell is a neural stem cell (NSC). In some embodiments, the engineered cell is a limbal stem cell (LSC). In some embodiments, the engineered cell is a progenitor cell, e.g. an endothelial progenitor cell or a neural progenitor cell. In some embodiments, the engineered cell is a tissue-specific primary cell. In some embodiments, the engineered cell is chosen from: chondrocyte, myocyte, and keratinocyte. In some embodiments, the engineered cell is a monocyte, macrophage, mast cell, dendritic cell, or granulocyte. In some embodiments, the engineered cell is monocyte. In some embodiments, the engineered cell is a macrophage. In some embodiments, the engineered cell is a mast cell. In some embodiments, the engineered cell is a dendritic cell. In some embodiments, the engineered cell is a granulocyte. In some embodiments, the engineered cell is a lymphocyte. In some embodiments, the engineered cell is a T cell. In some embodiments, the engineered cell is a CD4+ T cell. In some embodiments, the engineered cell is a CD8+ T cell. In some embodiments, the engineered cell is a memory T cell. In some embodiments, the engineered cell is a B cell. In some embodiments, the engineered cell is a plasma B cell. In some embodiments, the engineered cell is a memory B cell. In some embodiments, the engineered cell is a macrophage.

In some embodiments, the disclosure provides a pharmaceutical composition comprising any one of the engineered human cells disclosed herein. In some embodiments, the pharmaceutical composition comprises a population of any one of the engineered cells disclosed herein. In some embodiments, the population of engineered cells is at least 65% HLA-B negative as measured by flow cytometry. In some embodiments, the population of engineered cells is at least 70% HLA-B negative as measured by flow cytometry. In some embodiments, the population of engineered cells is at least 80% HLA-B negative as measured by flow cytometry. In some embodiments, the population of engineered cells is at least 90% HLA-B negative as measured by flow cytometry. In some embodiments, the population of engineered cells is at least 91% negative as measured by flow cytometry. In some embodiments, the population of engineered cells that is at least 92% HLA-B negative as measured by flow cytometry. In some embodiments, the population of engineered cells is at least 93% HLA-B negative as measured by flow cytometry. In some embodiments, the population of engineered cells is at least 94% HLA-B negative as measured by flow cytometry.

In some embodiments, the population of cells is at least 94% HLA-A negative or at least 94% HLA-B negative, as measured by flow cytometry. In some embodiments, the population of cells is at least 95% HLA-A negative or at least 95% HLA-B negative, as measured by flow cytometry. In some embodiments, the population of cells is at least 96% HLA-A negative or at least 96% HLA-B negative, as measured by flow cytometry. In some embodiments, the population of cells is at least 97% HLA-A negative or at least 97% HLA-B negative, as measured by flow cytometry. In some embodiments, the population of cells is at least 98% HLA-A negative or at least 98% HLA-B negative as measured by flow cytometry. In some embodiments, the population of cells is at least 99% HLA-A negative or at least 98% HLA-B negative as measured by flow cytometry.

In some embodiments, the population of cells is at least 94% HLA-A negative and at least 94% HLA-B negative, as measured by flow cytometry. In some embodiments, the population of cells is at least 95% HLA-A negative and at least 95% HLA-B negative, as measured by flow cytometry. In some embodiments, the population of cells is at least 96% HLA-A negative and at least 96% HLA-B negative, as measured by flow cytometry. In some embodiments, the population of cells is at least 97% HLA-A negative and at least 97% HLA-B negative, as measured by flow cytometry. In some embodiments, the population of cells is at least 98% HLA-A negative and at least 98% HLA-B negative as measured by flow cytometry. In some embodiments, the population of cells is at least 99% HLA-A negative and at least 98% HLA-B negative as measured by flow cytometry.

In some embodiments, at least 92% of the population of cells comprises the genetic modification in the HLA-A gene or 92% of the population of cells comprises the genetic modification in the HLA-B gene, as measured by next-generation sequencing (NGS). In some embodiments, the population of cells is at least 93% HLA-A negative or at least 93% HLA-B negative, as measured by flow cytometry. In some embodiments, at least 93% of the population of cells comprises the genetic modification in the HLA-A gene or at least 93% of the population of cells comprises the genetic modification in the HLA-B gene, as measured by next-generation sequencing (NGS). In some embodiments, at least 94% of the population of cells comprises the genetic modification in the HLA-A gene or at least 94% of the population of cells comprises the genetic modification in the HLA-B gene, as measured by next-generation sequencing (NGS). In some embodiments, at least 95% of the population of cells comprises the genetic modification in the HLA-A gene or at least 95% of the population of cells comprises the genetic modification in the HLA-B gene, as measured by next-generation sequencing (NGS). In some embodiments, at least 96% of the population of cells comprises the genetic modification in the HLA-A gene or at least 96% of the population of cells comprises the genetic modification in the HLA-B gene, as measured by next-generation sequencing (NGS). In some embodiments, at least 96% of the population of cells comprises the genetic modification in the HLA-A gene or at least 97% of the population of cells comprises the genetic modification in the HLA-B gene, as measured by next-generation sequencing (NGS). In some embodiments, at least 96% of the population of cells comprises the genetic modification in the HLA-A gene or at least 98% of the population of cells comprises the genetic modification in the HLA-B gene, as measured by next-generation sequencing (NGS). In some embodiments, at least 96% of the population of cells comprises the genetic modification in the HLA-A gene or at least 99% of the population of cells comprises the genetic modification in the HLA-B gene, as measured by next-generation sequencing (NGS).

In some embodiments, the population of cells is at least 95% CIITA negative as measured by flow cytometry. In some embodiments, the population of cells is at least 96% CIITA negative as measured by flow cytometry. In some embodiments, the population of cells is at least 97% CIITA negative as measured by flow cytometry. In some embodiments, the population of cells is at least 98% CIITA negative as measured by flow cytometry. In some embodiments, the population of cells is at least 99% CIITA negative as measured by flow cytometry.

In some embodiments, the population of engineered cells is at least 95% endogenous TCR protein negative as measured by flow cytometry. In some embodiments, the population of engineered cells is at least 97% endogenous TCR protein negative as measured by flow cytometry. In some embodiments, the population of engineered cells is at least 98% endogenous TCR protein negative as measured by flow cytometry. In some embodiments, the population of engineered cells is at least 99% endogenous TCR protein negative as measured by flow cytometry. In some embodiments, the population of engineered cells is at least 99.5% endogenous TCR protein negative as measured by flow cytometry.

In some embodiments, methods are provided for administering the engineered human cells or pharmaceutical compositions disclosed herein to a subject in need thereof. In some embodiments, methods are provided for administering the engineered human cells or pharmaceutical compositions disclosed herein to a subject as an ACT therapy. In some embodiments, methods are provided for administering the engineered human cells or pharmaceutical compositions disclosed herein to a subject as a treatment for cancer. In some embodiments, methods are provided for administering the engineered human cells or pharmaceutical compositions disclosed herein to a subject as a treatment for an autoimmune disease. In some embodiments, methods are provided for administering the engineered human cells or pharmaceutical compositions disclosed herein to a subject as a treatment for an infectious disease.

### C. Methods and Compositions for Reducing or Eliminating Surface Expression of HLA-B

The present disclosure provides methods and compositions for reducing or eliminating surface expression of HLA-B protein relative to an unmodified cell by genetically modifying the HLA-B gene. The disclosure also provides methods and compositions for reducing or eliminating surface expression of both HLA-A and HLA-B protein relative to an unmodified cell by genetically modifying the HLA-A and HLA-B genes. The resultant genetically modified cell may also be referred to herein as an engineered cell. In some embodiments, an already-genetically modified (or engineered) cell may be the starting cell for further genetic modification using the methods or compositions provided herein. In some embodiments, the cell is an allogeneic cell. In some embodiments, a cell with reduced or eliminated surface expression of HLA-B protein only or HLA-A and HLA-B protein is useful for adoptive cell transfer therapies. In some embodiments, editing of the HLA-A or HLA-B gene is combined with additional genetic modifications to yield a cell that is desirable for allogeneic transplant purposes.

In some embodiments, the methods comprise reducing surface expression of HLA-B protein in a human cell relative to an unmodified cell, comprising contacting a cell with composition comprising (a) a guide RNA comprising (i) a guide sequence selected from SEQ ID NOs: 1-91 and 101-185; or (ii) at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or at least 95%, 90%, 85%, 80%, 75%, or 70% identical to a sequence selected from SEQ ID NOs: 101-185; or (iv) a guide sequence that binds a target site comprising a genomic region listed in Table 2 or 3; or (v) a guide sequence that is complementary to at least 17, 18, 19, or 20 contiguous nucleotides of a genomic region listed in Table 2, or a guide sequence that is complementary to at least 17, 18, 19, 20, 21, 22, 23, or 24 contiguous nucleotides of a genomic region listed in Table 3; or (vi) a guide sequence that is at least 95%, 90%, 85%, 80%, 75%, or 70% identical to a sequence selected from (v); and optionally (b) an RNA-guided DNA binding agent or a nucleic acid encoding an RNA-guided DNA binding agent.

In some embodiments, the methods further comprise contacting the cell with an RNA-guided DNA binding agent or a nucleic acid encoding an RNA-guided DNA binding agent. In some embodiments, the RNA-guided DNA binding agent comprises a Cas9 protein. In some embodiments, the RNA-guided DNA binding agent is selected from one of S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX. In some embodiments, the RNA-guided DNA binding agent comprises a polypeptide selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments the RNA-guided DNA binding agent is N. meningitidis Cas9, e.g., Nme2Cas9. In some embodiments the RNA-guided DNA binding agent is S. thermophilus Cas9. In some embodiments the RNA-guided DNA binding agent is S. aureus Cas9. In some embodiments the RNA-guided DNA binding agent is Cpf1 from F. novicida. In some embodiments the RNA-guided DNA binding agent is Cpf1 from Acidaminococcus sp. In some embodiments the RNA-guided DNA binding agent is Cpf1 from Lachnospiraceae bacterium ND2006. In some embodiments the RNA-guided DNA binding agent is a C to T base editor. In some embodiments the RNA-guided DNA binding agent is a A to G base editor. In some embodiments, the base editor comprises a deaminase and an RNA-guided nickase. In some embodiments the RNA-guided DNA binding agent comprises a APOBEC3A deaminase (A3A) and an RNA-guided nickase. In some embodiments, the RNA-guided nickase is a SpyCas9 nickase. In some embodiments, the RNA-guided nickase comprises an NmeCas9 nickase. In some embodiments the RNA-guided DNA binding agent is Cas12a. In some embodiments the RNA-guided DNA binding agent is CasX. In some embodiments, the surface expression of HLA-A protein (i.e., engineered cell) is thereby reduced or eliminated.

In some embodiments, the methods comprise reducing surface expression of HLA-A and HLA-B protein in a human cell relative to an unmodified cell, comprising contacting a cell with composition comprising (a) an HLA-A guide RNA comprising (i) a guide sequence selected from SEQ ID NOs: 301-590; or (ii) at least 17, 18, 19, or 20 contiguous nucleotides of a sequence selected from SEQ ID NOs: 301-428 and 463-511; or at least 17, 18, 19, 20, 21, 22, 23, or 24 contiguous nucleotides of a sequence selected from SEQ ID NOs 429-462 and 512-590; or (iii) a guide sequence at least 95%, 90%, or 85% identical to a sequence selected from SEQ ID NOs: 301-428 and 463-511; or at least 95%, 90%, 85%, 80%, 75%, or 70% identical to a sequence selected from SEQ ID NOs: 429-462 and 512-590; or (iv) a guide sequence that binds a target site comprising a genomic region listed in Tables 4-7; or (v) a guide sequence that is complementary to at least 17, 18, 19, or 20 contiguous nucleotides of a genomic region listed in Table 4, Table 5B, or Table 6, or a guide sequence that is complementary to at least 17, 18, 19, 20, 21, 22, 23, or 24 contiguous nucleotides of a genomic region listed in Table 5A or Table 7; or (vi) a guide sequence that is at least 95%, 90%, 85%, 80%, 75%, or 70% identical to a sequence selected from (v); and optionally (b) a first RNA-guided DNA binding agent or a nucleic acid encoding an RNA-guided DNA binding agent; and contacting a cell with a second composition comprising (a) an HLA-B guide RNA comprising (i) a guide sequence selected from SEQ ID NOs: 1-91 and 101-185; or (ii) at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or at least 95%, 90%, 85%, 80%, 75%, or 70% identical to a sequence selected from SEQ ID NOs: 101-185; or (iv) a guide sequence that binds a target site comprising a genomic region listed in Table 2 or 3; or (v) a guide sequence that is complementary to at least 17, 18, 19, or 20 contiguous nucleotides of a genomic region listed in Table 2, or a guide sequence that is complementary to at least 17, 18, 19, 20, 21, 22, 23, or 24 contiguous nucleotides of a genomic region listed in Table 3; or (vi) a guide sequence that is at least 95%, 90%, 85%, 80%, 75%, or 70% identical to a sequence selected from (v); and optionally (b) an RNA-guided DNA binding agent or a nucleic acid encoding an RNA-guided DNA binding agent. In some embodiments, the methods further comprise contacting the cell with an RNA-guided DNA binding agent or a nucleic acid encoding an RNA-guided DNA binding agent. In some embodiments, the RNA-guided DNA binding agent comprises a Cas9 protein. In some embodiments, the RNA-guided DNA binding agent is selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX. In some embodiments, the RNA-guided DNA binding agent comprises a polypeptide selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments the RNA-guided DNA binding agent is N. meningitidis Cas9, e.g., Nme2Cas9. In some embodiments the RNA-guided DNA binding agent is S. thermophilus Cas9. In some embodiments the RNA-guided DNA binding agent is S. aureus Cas9. In some embodiments the RNA-guided DNA binding agent is Cpf1 from F. novicida. In some embodiments the RNA-guided DNA binding agent is Cpf1 from Acidaminococcus sp. In some embodiments the RNA-guided DNA binding agent is Cpf1 from Lachnospiraceae bacterium ND2006. In some embodiments the RNA-guided DNA binding agent is a C to T base editor. In some embodiments the RNA-guided DNA binding agent is a A to G base editor. In some embodiments, the base editor comprises a deaminase and an RNA-guided nickase. In some embodiments the RNA-guided DNA binding agent comprises a APOBEC3A deaminase (A3A) and an RNA-guided nickase. In some embodiments, the RNA-guided nickase is a SpyCas9 nickase. In some embodiments, the RNA-guided nickase comprises an NmeCas9 nickase. In some embodiments the RNA-guided DNA binding agent is Cas12a. In some embodiments the RNA-guided DNA binding agent is CasX. In some embodiments, the surface expression of HLA-A protein (i.e., engineered cell) is thereby reduced or eliminated.

In some embodiments, the methods comprise making an engineered human cell, which has reduced or eliminated surface expression of HLA-B protein relative to an unmodified cell, wherein the cell is homozygous for HLA-A and homozygous for HLA-C, comprising contacting a cell with composition comprising (a) a guide RNA comprising (i) a guide sequence selected from SEQ ID NOs: 1-91 and 101-185; or (ii) at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or at least 95%, 90%, 85%, 80%, 75%, or 70% identical to a sequence selected from SEQ ID NOs: 101-185; or (iv) a guide sequence that binds a target site comprising a genomic region listed in Table 2 or 3; or (v) a guide sequence that is complementary to at least 17, 18, 19, or 20 contiguous nucleotides of a genomic region listed in Table 2, or a guide sequence that is complementary to at least 17, 18, 19, 20, 21, 22, 23, or 24 contiguous nucleotides of a genomic region listed in Table 3; or (vi) a guide sequence that is at least 95%, 90%, 85%, 80%, 75%, or 70% identical to a sequence selected from (v); and optionally (b) an RNA-guided DNA binding agent or a nucleic acid encoding an RNA-guided DNA binding agent. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes or N. meningitidis (e.g., Nme2) Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments, the surface expression of HLA-A B protein (i.e., engineered cell) is thereby reduced or eliminated.

In some embodiments, the methods comprise making an engineered human cell, which has reduced or eliminated surface expression of HLA-A and HLA-B protein relative to an unmodified cell, wherein the cell is homozygous for HLA-A and homozygous for HLA-C, comprising contacting a cell with composition comprising (a) an HLA-A guide RNA comprising (i) a guide sequence selected from SEQ ID NOs: 301-590; or (ii) at least 17, 18, 19, or 20 contiguous nucleotides of a sequence selected from SEQ ID NOs: 301-428 and 463-511; or at least 17, 18, 19, 20, 21, 22, 23, or 24 contiguous nucleotides of a sequence selected from SEQ ID NOs 429-462 and 512-590; or (iii) a guide sequence at least 95%, 90%, or 85% identical to a sequence selected from SEQ ID NOs: 301-428 and 463-511; or at least 95%, 90%, 85%, 80%, 75%, or 70% identical to a sequence selected from SEQ ID NOs: 429-462 and 512-590; or (iv) a guide sequence that binds a target site comprising a genomic region listed in Tables 4-7; or (v) a guide sequence that is complementary to at least 17, 18, 19, or 20 contiguous nucleotides of a genomic region listed in Tables 4, 5B and 6, or a guide sequence that is complementary to at least 17, 18, 19, 20, 21, 22, 23, or 24 contiguous nucleotides of a genomic region listed in Table 5A or Table 7; or (vi) a guide sequence that is at least 95%, 90%, 85%, 80%, 75%, or 70% identical to a sequence selected from (v); and optionally (b) a first RNA-guided DNA binding agent or a nucleic acid encoding an RNA-guided DNA binding agent; and contacting a cell with a second composition comprising (a) an HLA-B guide RNA comprising (i) a guide sequence selected from SEQ ID NOs: 1-91 and 101-185; or (ii) at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or at least 95%, 90%, 85%, 80%, 75%, or 70% identical to a sequence selected from SEQ ID NOs: 101-185; or (iv) a guide sequence that binds a target site comprising a genomic region listed in Table 2 or 3; or (v) a guide sequence that is complementary to at least 17, 18, 19, or 20 contiguous nucleotides of a genomic region listed in Table 2, or a guide sequence that is complementary to at least 17, 18, 19, 20, 21, 22, 23, or 24 contiguous nucleotides of a genomic region listed in Table 3; or (vi) a guide sequence that is at least 95%, 90%, 85%, 80%, 75%, or 70% identical to a sequence selected from (v); and optionally (b) an RNA-guided DNA binding agent or a nucleic acid encoding an RNA-guided DNA binding agent. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes or N. meningitidis (e.g., Nme2) Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments, the surface expression of HLA-B protein (i.e., engineered cell) is thereby reduced or eliminated.

In some embodiments, the methods of reducing or eliminating surface expression of HLA-A or HLA-B protein comprise contacting a cell with any one or more of the HLA-A or HLA-B guide RNAs disclosed herein.

In some embodiments, compositions are provided comprising a) an HLA-B guide RNA comprising: (i) a guide sequence selected from SEQ ID NOs: 1-91 or 101-185; or (ii). at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185; or (iii) a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185; or (iv) a guide sequence that binds a target site comprising a genomic region listed in Tables 2-3; or (v) a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3; or (vi) a guide sequence that is at least 95%, 90%, or 85%, 80%, 75%, or 70% identical to a sequence selected from (v); and optionally b) an RNA-guided DNA binding agent or a nucleic acid encoding an RNA-guided DNA binding agent. In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase.

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the HLA-A or HLA-B genomic target sequence. In some embodiments, the composition comprises an RNA-guided DNA binding agent that generates an adenosine (A) to guanine (G) conversion with the HLA-A or HLA-B genomic target sequence.

In some embodiments, an engineered human cell produced by the methods described herein is provided. In some embodiments, the engineered human cell produced by the methods and compositions described herein is an allogeneic cell. In some embodiments, the methods produce a composition comprising an engineered human cell having reduced or eliminated surface expression of HLA-A or HLA-B protein. In some embodiments, the engineered human cell produced by the methods disclosed herein elicits a reduced response from CD8+ T cells as compared to an unmodified cell as measured in an in vitro cell culture assay containing CD8+ T cells.

In some embodiments, the compositions disclosed herein further comprise a pharmaceutically acceptable carrier. In some embodiments, a cell produced by the compositions disclosed herein comprising a pharmaceutically acceptable carrier is provided. In some embodiments, compositions comprising the cells disclosed herein are provided.

### 1. HLA-B Guide RNAs

The methods and compositions provided herein disclose guide RNAs useful for reducing or eliminating the surface expression of HLA-B protein. In some embodiments, such guide RNAs direct an RNA-guided DNA binding agent to an HLA-A genomic target sequence and may be referred to herein as “HLA-B guide RNAs.” In some embodiments, the HLA-B guide RNA directs an RNA-guided DNA binding agent to a human HLA-B genomic target sequence. In some embodiments, the HLA-B guide RNA comprises a guide sequence selected from SEQ ID NOs: 1-91. In some embodiments, the HLA-B guide RNA comprises a guide sequence selected from SEQ ID NOs: 101-185.

In some embodiments, a composition is provided comprising an -B guide RNA described herein and an RNA-guided DNA binding agent or a nucleic acid encoding an RNA-guided DNA binding agent.

In some embodiments, a composition is provided comprising an HLA-B single-guide RNA (sgRNA) comprising a guide sequence selected from SEQ ID NOs: 1-91 or 101-185. In some embodiments, a composition is provided comprising HLA-B sgRNA described herein and an RNA-guided DNA binding agent or a nucleic acid encoding an RNA-guided DNA binding agent.

In some embodiments, a composition is provided comprising an HLA-B dual-guide RNA (dgRNA) comprising a guide sequence selected from SEQ ID NOs: 1-91 or 101-185. In some embodiments, a composition is provided comprising an HLA-B dgRNA described herein and an RNA-guided DNA binding agent or a nucleic acid encoding an RNA-guided DNA binding agent.

In some embodiments, the HLA-B gRNA comprises a guide sequence selected from any one of SEQ ID NOs: 1-91 or 101-185. Exemplary HLA-B guide sequences are shown below in Table 2 (SEQ ID NOs: 1-91), and Table 3 (SEQ ID NOs: 101-185).

In some embodiments, the HLA-A gRNA is a sgRNA comprising a sequence as shown below in Table 2 (SEQ ID NOs: 1001-1091 and 2001-2091), Table 3 (SEQ ID NOs: 1101-1185, and 2101-2185), and Table 3A (SEQ ID NOs: 2186-2191).

TABLE 2
Exemplary HLA-B Spy guide RNAs

| | SEQ ID | | Exemplary | Exemplary Guide | |
|---|---|---|---|---|---|
| | NO to | | Guide RNA | RNA Modified | |
| | the | | Full Sequence | Sequence | Genomic |
| Guide | Guide | Guide | (SEQ ID NOS: | (SEQ ID NOs: | Coordinates |
| ID | Sequence | Sequence | 1001-1091) | 2001-2091) | (hg38) |
| G022008 | 1 | CCAGCCUGGACG | CCAGCCUGG | mC*mC*mA*GCCUG | chr6:31355718- |
| | | CAGGCACC | ACGCAGGCA | GACGCAGGCACCG | 31355738 |
| | | | CCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022009 | 2 | AGGCGCUUUGCA | AGGCGCUUU | mA*mG*mG*CGCUU | chr6:31355616- |
| | | UCUCUCAU | GCAUCUCUC | UGCAUCUCUCAUG | 31355636 |
| | | | AUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022010 | 3 | AACAAUGCCCAC | AACAAUGCC | mA*mA*mC*AAUGC | chr6:31355182- |
| | | GAUGGGGA | CACGAUGGG | CCACGAUGGGGAG | 31355202 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022011 | 4 | CCAGACACCAGC | CCAGACACC | mC*mC*mA*GACAC | chr6:31355711- |
| | | CUGGACGC | AGCCUGGAC | CAGCCUGGACGCG | 31355731 |
| | | | GCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022012 | 5 | CCAUCUCCUAUA | CCAUCUCCU | mC*mC*mA*UCUCC | chr6:31356120- |
| | | GGUCGCCG | AUAGGUCGC | UAUAGGUCGCCGG | 31356140 |
| | | | CGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022013 | 6 | CUUCUCUCUAGG | CUUCUCUCU | mC*mU*mU*CUCUC | chr6:31270893- |
| | | ACAAUUAA | AGGACAAUU | UAGGACAAUUAAG | 31270913 |
| | | | AAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022014 | 7 | CCCCAUCUCCUA | CCCCAUCUC | mC*mC*mC*CAUCU | chr6:31356122- |
| | | UAGGUCGC | CUAUAGGUC | CCUAUAGGUCGCG | 31356142 |
| | | | GCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022015 | 8 | GGAUCUCGGACC | GGAUCUCGG | mG*mG*mA*UCUCG | chr6:31354563- |
| | | CGGAGACU | ACCCGGAGA | GACCCGGAGACUG | 31354583 |
| | | | CUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022016 | 9 | CUUCCUCCCAGU | CUUCCUCCC | mC*mU*mU*CCUCC | chr6:31354682- |
| | | CCCCUCAC | AGUCCCCUC | CAGUCCCCUCACG | 31354702 |
| | | | ACGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022017 | 10 | CUCCUGUCCCAC | CUCCUGUCC | mC*mU*mC*CUGUC | chr6:31355900- |
| | | GUCUCCUG | CACGUCUCC | CCACGUCUCCUGG | 31355920 |
| | | | UGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022018 | 11 | CCAUUUUCCUCC | CCAUUUUCC | mC*mC*mA*UUUUC | chr6:31356086- |
| | | UCUUCUCG | UCCUCUUCU | CUCCUCUUCUCGG | 31356106 |
| | | | CGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022019 | 12 | GGUUCCCAAGGC | GGUUCCCAA | mG*mG*mU*UCCCA | chr6:31356623- |
| | | UGCUGCAG | GGCUGCUGC | AGGCUGCUGCAGG | 31356643 |
| | | | AGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022020 | 13 | ACAUGCCAUGUA | ACAUGCCAU | mA*mC*mA*UGCCA | chr6:31355348- |
| | | CAGCAUGA | GUACAGCAU | UGUACAGCAUGAG | 31355368 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022021 | 14 | CGGUUCCCAAGG | CGGUUCCCA | mC*mG*mG*UUCCC | chr6:31355899- |
| | | CUGCUGCA | AGGCUGCUG | AAGGCUGCUGCAG | 31355919 |
| | | | CAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022022 | 15 | AACGCGCCUGGG | AACGCGCCU | mA*mA*mC*GCGCC | chr6:31356539- |
| | | GCUCUCGC | GGGGCUCUC | UGGGGCUCUCGCG | 31356559 |
| | | | GCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022023 | 16 | UGGUGGGGGUGG | UGGUGGGGG | mU*mG*mG*UGGG | chr6:31357031- |
| | | GAGUGUGG | UGGGAGUGU | GGUGGGAGUGUGG | 31357051 |
| | | | GGGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022024 | 17 | GAGUCUCUGAGC | GAGUCUCUG | mG*mA*mG*UCUCU | chr6:31355713- |
| | | GGGGAACA | AGCGGGGAA | GAGCGGGGAACAG | 31355733 |
| | | | CAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022025 | 18 | GGGAGGAGCGAG | GGGAGGAGC | mG*mG*mG*AGGA | chr6:31355180- |
| | | GGGACCGC | GAGGGGACC | GCGAGGGGACCGC | 31355200 |
| | | | GCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022026 | 19 | UGAGGGGACUGG | UGAGGGGAC | mU*mG*mA*GGGG | chr6:31356601- |
| | | GAGGAAGC | UGGGAGGAA | ACUGGGAGGAAGC | 31356621 |
| | | | GCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022027 | 20 | CUGCGUCCAGGC | CUGCGUCCA | mC*mU*mG*CGUCC | chr6:31270992- |
| | | UGGUGUCU | GGCUGGUGU | AGGCUGGUGUCUG | 31271012 |
| | | | CUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022028 | 21 | CCUCGACCGGCG | CCUCGACCG | mC*mC*mU*CGACC | chr6:31356548- |
| | | AGAGCCCC | GCGAGAGCC | GGCGAGAGCCCCG | 31356568 |
| | | | CCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022029 | 22 | AACACGAGGAAA | AACACGAGG | mA*mA*mC*ACGAG | chr6:31354795- |
| | | GCAAGUGU | AAAGCAAGU | GAAAGCAAGUGUG | 31354815 |
| | | | GUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022030 | 23 | ACUUUACCUCCA | ACUUUACCU | mA*mC*mU*UUACC | chr6:31355803- |
| | | CUCAGAUC | CCACUCAGA | UCCACUCAGAUCG | 31355823 |
| | | | UCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022031 | 24 | UCUGCUCCUGAU | UCUGCUCCU | mU*mC*mU*GCUCC | chr6:31356486- |
| | | CUGAGUGG | GAUCUGAGU | UGAUCUGAGUGGG | 31356506 |
| | | | GGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022032 | 25 | AUCUCGGACCCG | AUCUCGGAC | mA*mU*mC*UCGGA | chr6:31354820- |
| | | GAGACUCG | CCGGAGACU | CCCGGAGACUCGG | 31354840 |
| | | | CGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | | GmCmU*mU*mU*m | |
| | | | | U | |
| G022033 | 26 | CCUGGGGCUCUC | CCUGGGGCU | mC*mC*mU*GGGGC | chr6:31356545- |
| | | GCCGGUCG | CUCGCCGGU | UCUCGCCGGUCGG | 31356565 |
| | | | CGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022034 | 27 | CUGGUCACAUGG | CUGGUCACA | mC*mU*mG*GUCAC | chr6:31356501- |
| | | GUGGUCCU | UGGGUGGUC | AUGGGUGGUCCUG | 31356521 |
| | | | CUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022035 | 28 | CUCCUAUAGGUC | CUCCUAUAG | mC*mU*mC*CUAUA | chr6:31356116- |
| | | GCCGGGGA | GUCGCCGGG | GGUCGCCGGGGAG | 31356136 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022036 | 29 | GUAAAGUGACUC | GUAAAGUGA | mG*mU*mA*AAGU | chr6:31355816- |
| | | AGAAGUGC | CUCAGAAGU | GACUCAGAAGUGC | 31355836 |
| | | | GCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022037 | 30 | ACACAGGGGCUA | ACACAGGGG | mA*mC*mA*CAGGG | chr6:31354983- |
| | | ACGCAGCC | CUAACGCAG | GCUAACGCAGCCG | 31355003 |
| | | | CCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022038 | 31 | AUACUUCUGGAA | AUACUUCUG | mA*mU*mA*CUUCU | chr6:31354748- |
| | | AUUCCUUU | GAAAUUCCU | GGAAAUUCCUUUG | |
| | | | UUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | 31354768 |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022039 | 32 | GUCCUAGCAGUU | GUCCUAGCA | mG*mU*mC*CUAGC | chr6:31355150- |
| | | GUGGUCAU | GUUGUGGUC | AGUUGUGGUCAUG | 31355170 |
| | | | AUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022040 | 33 | CCCAUCUCCUAU | CCCAUCUCC | mC*mC*mC*AUCUC | chr6:31356121- |
| | | AGGUCGCC | UAUAGGUCG | CUAUAGGUCGCCG | 31356141 |
| | | | CCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022041 | 34 | CGACCUAUAGGA | CGACCUAUA | mC*mG*mA*CCUAU | chr6:31356123- |
| | | GAUGGGGA | GGAGAUGGG | AGGAGAUGGGGAG | 31356143 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022042 | 35 | GAAGUAUGACUA | GAAGUAUGA | mG*mA*mA*GUAU | chr6:31354761- |
| | | CAGACCCA | CUACAGACC | GACUACAGACCCA | 31354781 |
| | | | CAGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022043 | 36 | CUCCGAUGACCA | CUCCGAUGA | mC*mU*mC*CGAUG | chr6:31355145- |
| | | CAACUGCU | CCACAACUG | ACCACAACUGCUG | 31355165 |
| | | | CUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022044 | 37 | AGUUGUGGUCAU | AGUUGUGGU | mA*mG*mU*UGUG | chr6:31355142- |
| | | CGGAGCUG | CAUCGGAGC | GUCAUCGGAGCUG | 31355162 |
| | | | UGGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022045 | 38 | UACUUCUGGAAA | UACUUCUGG | mU*mA*mC*UUCUG | chr6:31354747- |
| | | UUCCUUUU | AAAUUCCUU | GAAAUUCCUUUUG | 31354767 |
| | | | UUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022046 | 39 | CACAUGCCAUGU | CACAUGCCA | mC*mA*mC*AUGCC | chr6:31355349- |
| | | ACAGCAUG | UGUACAGCA | AUGUACAGCAUGG | 31355369 |
| | | | UGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022047 | 40 | GGAGCGAGGGGA | GGAGCGAGG | mG*mG*mA*GCGA | chr6:31357027- |
| | | CCGCAGGC | GGACCGCAG | GGGGACCGCAGGC | 31357047 |
| | | | GCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022048 | 41 | AGGAAAAGUCAC | AGGAAAAGU | mA*mG*mG*AAAA | chr6:31355888- |
| | | GGUUCCCA | CACGGUUCC | GUCACGGUUCCCA | 31355908 |
| | | | CAGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022049 | 42 | GAGCCUUCCCCA | GAGCCUUCC | mG*mA*mG*CCUUC | chr6:31356129- |
| | | UCUCCUAU | CCAUCUCCU | CCCAUCUCCUAUG | 31356149 |
| | | | AUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022050 | 43 | GACUCCCCAUCC | GACUCCCCA | mG*mA*mC*UCCCC | chr6:31356636- |
| | | CCCACGUA | UCCCCCACG | AUCCCCCACGUAG | 31356656 |
| | | | UAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022051 | 44 | CCCGGCGACCUA | CCCGGCGAC | mC*mC*mC*GGCGA | chr6:31356118- |
| | | UAGGAGAU | CUAUAGGAG | CCUAUAGGAGAUG | 31356138 |
| | | | AUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022052 | 45 | ACGGUUCCCAAG | ACGGUUCCC | mA*mC*mG*GUUCC | chr6:31355898- |
| | | GCUGCUGC | AAGGCUGCU | CAAGGCUGCUGCG | 31355918 |
| | | | GCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G022053 | 46 | GGGAGUCGUGAC | GGGAGUCGU | mG*mG*mG*AGUC | chr6:31356649- |
| | | CUGCGCCC | GACCUGCGC | GUGACCUGCGCCC | 31356669 |
| | | | CCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022054 | 47 | GAGCGAGGGGAC | GAGCGAGGG | mG*mA*mG*CGAG | chr6:31357026- |
| | | CGCAGGCG | GACCGCAGG | GGGACCGCAGGCG | 31357046 |
| | | | CGGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCAmAmC | |
| | | | UAUCAACUU | mUmUmGmAmAmA | |
| | | | GAAAAAGUG | mAmAmGmUmGmG | |
| | | | GCACCGAGU | mCmAmCmCmGmA | |
| | | | CGGUGCUUU | mGmUmCmGmGmU | |
| | | | U | mGmCmU*mU*mU* | |
| | | | | mU | |
| G022055 | 48 | CCUGGCUGUCCU | CCUGGCUGU | mC*mC*mU*GGCUG | chr6:31355157- |
| | | AGCAGUUG | CCUAGCAGU | UCCUAGCAGUUGG | 31355177 |
| | | | UGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCAmAmCm | |
| | | | UAUCAACUU | UmUmGmAmAmAm | |
| | | | GAAAAAGUG | AmAmGmUmGmGm | |
| | | | GCACCGAGU | CmAmCmCmGmAm | |
| | | | CGGUGCUUU | GmUmCmGmGmUm | |
| | | | U | GmCmU*mU*mU*m | |
| | | | | U | |
| G027488 | 49 | ACAUGCCAUGUA | ACAUGCCAU | mA*mC*mA*UGCCA | chr6:31355348- |
| | | CAGCAUGA | GUACAGCAU | UGUACAGCAUGAG | 31355368 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027489 | 50 | AACAAUGCCCAC | AACAAUGCC | mA*mA*mC*AAUGC | chr6:31355182- |
| | | GAUGGGGA | CACGAUGGG | CCACGAUGGGGAG | 31355202 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027490 | 51 | CUCCGAUGACCA | CUCCGAUGA | mC*mU*mC*CGAUG | chr6:31355145- |
| | | CAACUGCU | CCACAACUG | ACCACAACUGCUG | 31355165 |
| | | | CUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027491 | 52 | CACAUGCCAUGU | CACAUGCCA | mC*mA*mC*AUGCC | chr6:31355349- |
| | | ACAGCAUG | UGUACAGCA | AUGUACAGCAUGG | 31355369 |
| | | | UGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027973 | 53 | CCGGACGGGCGC | CCGGACGGG | mC*mC*mG*GACGG | chr6:31356381- |
| | | CUCCUCCG | CGCCUCCUC | GCGCCUCCUCCGG | 31356401 |
| | | | CGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027974 | 54 | CGGACGGGCGCC | CGGACGGGC | mC*mG*mG*ACGGG | chr6:31356380- |
| | | UCCUCCGC | GCCUCCUCC | CGCCUCCUCCGCG | 31356400 |
| | | | GCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027975 | 55 | GAGCCGUCUUCC | GUGGACUGG | mG*mU*mG*GACU | chr6:31355204- |
| | | CAGUCCAC | GAAGACGGC | GGGAAGACGGCUC | 31355224 |
| | | | UCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G027976 | 56 | AGAGCCGUCUUC | UGGACUGGG | mU*mG*mG*ACUG | chr6:31355205- |
| | | CCAGUCCA | AAGACGGCU | GGAAGACGGCUCU | 31355225 |
| | | | CUGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G027977 | 57 | GUACGGCUGCGA | GUACGGCUG | mG*mU*mA*CGGCU | chr6:31356400- |
| | | CGUGGGGC | CGACGUGGG | GCGACGUGGGGCG | 31356420 |
| | | | GCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027978 | 58 | CCGUCCCCAUCG | AAUGCCCAC | mA*mA*mU*GCCCA | chr6:31355185- |
| | | UGGGCAUU | GAUGGGGAC | CGAUGGGGACGGG | 31355205 |
| | | | GGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027979 | 59 | UCGCUGCUGUGA | UCGCUGCUG | mU*mC*mG*CUGCU | chr6:31355119- |
| | | UGUGUAGG | UGAUGUGUA | GUGAUGUGUAGGG | 31355139 |
| | | | GGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027980 | 60 | UGGUCGCUGCUG | UGGUCGCUG | mU*mG*mG*UCGCU | chr6:31355122- |
| | | UGAUGUGU | CUGUGAUGU | GCUGUGAUGUGUG | 31355142 |
| | | | GUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027981 | 61 | CCAGGCAGCGAC | CCAGGCAGC | mC*mC*mA*GGCAG | chr6:31354510- |
| | | AGUGCCCA | GACAGUGCC | CGACAGUGCCCAG | 31354530 |
| | | | CAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027982 | 62 | GGCAGCGACAGU | CCCUGGGCA | mC*mC*mC*UGGGC | chr6:31354507- |
| | | GCCCAGGG | CUGUCGCUG | ACUGUCGCUGCCG | 31354527 |
| | | | CCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027983 | 63 | UCCAGGCAGCGA | UCCAGGCAG | mU*mC*mC*AGGCA | chr6:31354511- |
| | | CAGUGCCC | CGACAGUGC | GCGACAGUGCCCG | 31354531 |
| | | | CCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027984 | 64 | AGUCCACCGUCC | CACGAUGGG | mC*mA*mC*GAUGG | chr6:31355191- |
| | | CCAUCGUG | GACGGUGGA | GGACGGUGGACUG | 31355211 |
| | | | CUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027985 | 65 | CAGUCCACCGUC | CAGUCCACC | mC*mA*mG*UCCAC | chr6:31355192- |
| | | CCCAUCGU | GUCCCCAUC | CGUCCCCAUCGUG | 31355212 |
| | | | GUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027986 | 66 | GUCCACCGUCCC | CCACGAUGG | mC*mC*mA*CGAUG | chr6:31355190- |
| | | CAUCGUGG | GGACGGUGG | GGGACGGUGGACG | 31355210 |
| | | | ACGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027987 | 67 | CCAGUCCACCGU | CCAGUCCAC | mC*mC*mA*GUCCA | chr6:31355193- |
| | | CCCCAUCG | CGUCCCCAU | CCGUCCCCAUCGG | 31355213 |
| | | | CGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027988 | 68 | UCUUCCCAGUCC | GGGACGGUG | mG*mG*mG*ACGG | chr6:31355198- |
| | | ACCGUCCC | GACUGGGAA | UGGACUGGGAAGA | 31355218 |
| | | | GAGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G027989 | 69 | CGAAGCCCCUCA | CGAAGCCCC | mC*mG*mA*AGCCC | chr6:31355320- |
| | | CCCUGAGA | UCACCCUGA | CUCACCCUGAGAG | 31355340 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027990 | 70 | GAAGCCCCUCAC | GAAGCCCCU | mG*mA*mA*GCCCC | chr6:31355319- |
| | | CCUGAGAU | CACCCUGAG | UCACCCUGAGAUG | 31355339 |
| | | | AUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027991 | 71 | UCCCCAUCGUGG | AACAAUGCC | mA*mA*mC*AAUGC | chr6:31355182- |
| | | GCAUUGUU | CACGAUGGG | CCACGAUGGGGAG | 31355202 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027992 | 72 | AGUUGUGGUCAU | AGUUGUGGU | mA*mG*mU*UGUG | chr6:31355142- |
| | | CGGAGCUG | CAUCGGAGC | GUCAUCGGAGCUG | 31355162 |
| | | | UGGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G027993 | 73 | CAUCGUGGGCAU | CAGCAACAA | mC*mA*mG*CAACA | chr6:31355178- |
| | | UGUUGCUG | UGCCCACGA | AUGCCCACGAUGG | 31355198 |
| | | | UGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027994 | 74 | CAUGCCAUGUAC | CAUGCCAUG | mC*mA*mU*GCCAU | chr6:31355347- |
| | | AGCAUGAG | UACAGCAUG | GUACAGCAUGAGG | 31355367 |
| | | | AGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027995 | 75 | GGCUGUCCUAGC | CCACAACUG | mC*mC*mA*CAACU | chr6:31355154- |
| | | AGUUGUGG | CUAGGACAG | GCUAGGACAGCCG | 31355174 |
| | | | CCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027996 | 76 | AUCGUGGGCAUU | CCAGCAACA | mC*mC*mA*GCAAC | chr6:31355177- |
| | | GUUGCUGG | AUGCCCACG | AAUGCCCACGAUG | 31355197 |
| | | | AUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027997 | 77 | CCCAUCGUGGGC | CCCAUCGUG | mC*mC*mC*AUCGU | chr6:31355180- |
| | | AUUGUUGC | GGCAUUGUU | GGGCAUUGUUGCG | 31355200 |
| | | | GCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027998 | 78 | CCUGGCUGUCCU | CCUGGCUGU | mC*mC*mU*GGCUG | chr6:31355157- |
| | | AGCAGUUG | CCUAGCAGU | UCCUAGCAGUUGG | 31355177 |
| | | | UGGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G027999 | 79 | CGUGGGCAUUGU | CGUGGGCAU | mC*mG*mU*GGGCA | chr6:31355175- |
| | | UGCUGGCC | UGUUGCUGG | UUGUUGCUGGCCG | 31355195 |
| | | | CCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G028000 | 80 | AGCAGUUGUGGU | CUCCGAUGA | mC*mU*mC*CGAUG | chr6:31355145- |
| | | CAUCGGAG | CCACAACUG | ACCACAACUGCUG | 31355165 |
| | | | CUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G028001 | 81 | CUUGUGGAGACC | CUUGUGGAG | mC*mU*mU*GUGG | chr6:31355432- |
| | | AGACCAGC | ACCAGACCA | AGACCAGACCAGC | 31355452 |
| | | | GCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G028002 | 82 | UGUACAGCAUGA | GCAGCCCCU | mG*mC*mA*GCCCC | chr6:31355340- |
| | | GGGGCUGC | CAUGCUGUA | UCAUGCUGUACAG | 31355360 |
| | | | CAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G028003 | 83 | UCGUGGGCAUUG | GCCAGCAAC | mG*mC*mC*AGCAA | chr6:31355176- |
| | | UUGCUGGC | AAUGCCCAC | CAAUGCCCACGAG | 31355196 |
| | | | GAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G028004 | 84 | GUCCUAGCAGUU | GUCCUAGCA | mG*mU*mC*CUAGC | chr6:31355150- |
| | | GUGGUCAU | GUUGUGGUC | AGUUGUGGUCAUG | 31355170 |
| | | | AUGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G028005 | 85 | GUGUCUCUCACA | GUGUCUCUC | mG*mU*mG*UCUCU | chr6:31354480- |
| | | GCUUGAAA | ACAGCUUGA | CACAGCUUGAAAG | 31354500 |
| | | | AAGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G028006 | 86 | UCAGACCCCCCA | UGUGUCUUU | mU*mG*mU*GUCU | chr6:31355576- |
| | | AAGACACA | GGGGGGUCU | UUGGGGGGUCUGA | 31355596 |
| | | | GAGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G028007 | 87 | CUCCUCAGACGC | CUCCUCAGA | mC*mU*mC*CUCAG | chr6:31357154- |
| | | CGAGAUGC | CGCCGAGAU | ACGCCGAGAUGCG | 31357174 |
| | | | GCGUUUUAG | UUUUAGAmGmCmU | |
| | | | AGCUAGAAA | mAmGmAmAmAmU | |
| | | | UAGCAAGUU | mAmGmCAAGUUAA | |
| | | | AAAAUAAGG | AAUAAGGCUAGUC | |
| | | | CUAGUCCGU | CGUUAUCACGAAA | |
| | | | UAUCACGAA | GGGCACCGAGUCG | |
| | | | AGGGCACCG | GmU*mG*mC*mU | |
| | | | AGUCGGUGC | | |
| | | | U | | |
| G028008 | 88 | GAGAUAGAACCU | GAGAUAGAA | mG*mA*mG*AUAG | chr6:31355410- |
| | | UCCAGAAG | CCUUCCAGA | AACCUUCCAGAAG | 31355430 |
| | | | AGGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G028009 | 89 | GACCAGCAGGAG | GGUUCUAUC | mG*mG*mU*UCUA | chr6:31355419- |
| | | AUAGAACC | UCCUGCUGG | UCUCCUGCUGGUC | 31355439 |
| | | | UCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G028010 | 90 | GCAGGAGAUAGA | UGGAAGGUU | mU*mG*mG*AAGG | chr6:31355414- |
| | | ACCUUCCA | CUAUCUCCU | UUCUAUCUCCUGC | 31355434 |
| | | | GCGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |
| G028011 | 91 | AGAUAGAACCUU | AGAUAGAAC | mA*mG*mA*UAGA | chr6:31355409- |
| | | CCAGAAGU | CUUCCAGAA | ACCUUCCAGAAGU | 31355429 |
| | | | GUGUUUUAG | GUUUUAGAmGmCm | |
| | | | AGCUAGAAA | UmAmGmAmAmAm | |
| | | | UAGCAAGUU | UmAmGmCAAGUUA | |
| | | | AAAAUAAGG | AAAUAAGGCUAGU | |
| | | | CUAGUCCGU | CCGUUAUCACGAA | |
| | | | UAUCACGAA | AGGGCACCGAGUC | |
| | | | AGGGCACCG | GGmU*mG*mC*mU | |
| | | | AGUCGGUGC | | |
| | | | U | | |

TABLE 3
Exemplary N. meningitidis (Nme) HLA-B guide RNAs

| | SEQ ID | | Exemplary | Exemplary Guide | |
|---|---|---|---|---|---|
| | NO to | | Guide RNA Full | RNA Modified | |
| | the | | Sequence | Sequence | Genomic |
| | Guide | | (SEQ ID NOs: | (SEQ ID NOs: | Coordinates |
| Guide ID | Sequence | Guide Sequence | 1101-1185) | 2101-2185) | (hg38) |
| G028789 | 101 | CUUCUGGAG | CUUCUGGAG | mC*mU*mU*mCmUG | chr6: 31355361- |
| | | AAGAGCAGA | AAGAGCAGA | GmAmGAmAGmAGC | 31355385 |
| | | GAUACA | GAUACAGUU | AGmAGAUmACAmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028790 | 102 | CACAUGCCAU | CACAUGCCAU | mC*mA*mC*mAmUG | chr6: 31355339- |
| | | GUACAGCAU | GUACAGCAU | CmCmAUmGUmACA | 31355363 |
| | | GAGGG | GAGGGGUUG | GCmAUGAmGGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028791 | 103 | CACUGACCUG | CACUGACCUG | mC*mA*mC*mUmGA | chr6: 31355469- |
| | | GCAGCGGGA | GCAGCGGGA | CmCmUGmGCmAGC | 31355493 |
| | | UGGCG | UGGCGGUUG | GGmGAUGmGCGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028792 | 104 | CCAUGUACA | CCAUGUACA | mC*mC*mA*mUmGU | chr6: 31355333- |
| | | GCAUGAGGG | GCAUGAGGG | AmCmAGmCAmUGA | 31355357 |
| | | GCUGCC | GCUGCCGUU | GGmGGCUmGCCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028793 | 105 | CUUACACGCA | CUUACACGCA | mC*mU*mU*mAmCA | chr6: 31354627- |
| | | GCCUGAGAG | GCCUGAGAG | CmGmCAmGCmCUG | 31354651 |
| | | UAGCU | UAGCUGUUG | AGmAGUAmGCUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028794 | 106 | AGCUGCCCAC | AGCUGCCCAC | mA*mG*mC*mUmGC | chr6: 31355401- |
| | | UUCUGGAAG | UUCUGGAAG | CmCmACmUUmCUG | 31355425 |
| | | GUUCU | GUUCUGUUG | GAmAGGUmUCUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028795 | 107 | CUUCCAGAA | CUUCCAGAA | mC*mU*mU*mCmCA | chr6: 31355390- |
| | | GUGGGCAGC | GUGGGCAGC | GmAmAGmUGmGGC | 31355414 |
| | | UGUGGU | UGUGGUGUU | AGmCUGUmGGUmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028796 | 108 | CCAUCUCAGG | CCAUCUCAGG | mC*mC*mA*mUmCU | chr6: 31355317- |
| | | GUGAGGGGC | GUGAGGGGC | CmAmGGmGUmGAG | 31355341 |
| | | UUCGG | UUCGGGUUG | GGmGCUUmCGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028797 | 109 | CAUCUCAGG | CAUCUCAGG | mC*mA*mU*mCmUC | chr6: 31355318- |
| | | GUGAGGGGC | GUGAGGGGC | AmGmGGmUGmAGG | 31355342 |
| | | UUCGGC | UUCGGCGUU | GGmCUUCmGGCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028798 | 110 | CAGCAUGAG | CAGCAUGAG | mC*mA*mG*mCmAU | chr6: 31355326- |
| | | GGGCUGCCG | GGGCUGCCG | GmAmGGmGGmCUG | 31355350 |
| | | AAGCCC | AAGCCCGUU | CCmGAAGmCCCmG | |
| | | | GUAGCUCCCU | UUGmUmAmGmCUC | |
| | | | GAAACCGUU | CCmUmGmAmAmAm | |
| | | | | CmCGUUmGmCUAm | |
| | | | GCUACAAUA | CAAU*AAGmGmCC | |
| | | | AGGCCGUCG | mGmUmCmGmAmA | |
| | | | AAAGAUGUG | mAmGmAmUGUGCm | |
| | | | CCGCAACGCU | CGmCAAmCGCUCU | |
| | | | CUGCCUUCUG | mGmCCmUmUmCmU | |
| | | | GCAUCGUU | GGCAUCG*mU*mU | |
| G028799 | 111 | CCCAUCUCAG | CCCAUCUCAG | mC*mC*mC*mAmUC | chr6: 31355316- |
| | | GGUGAGGGG | GGUGAGGGG | UmCmAGmGGmUGA | 31355340 |
| | | CUUCG | CUUCGGUUG | GGmGGCUmUCGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028800 | 112 | AUGUACAGC | AUGUACAGC | mA*mU*mG*mUmAC | chr6: 31355331- |
| | | AUGAGGGGC | AUGAGGGGC | AmGmCAmUGmAGG | 31355355 |
| | | UGCCGA | UGCCGAGUU | GGmCUGCmCGAmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028801 | 113 | CAUGUACAG | CAUGUACAG | mC*mA*mU*mGmUA | chr6: 31355332- |
| | | CAUGAGGGG | CAUGAGGGG | CmAmGCmAUmGAG | 31355356 |
| | | CUGCCG | CUGCCGGUU | GGmGCUGmCCGmG | |
| | | | GUAGCUCCCU | | |
| | | | GAAACCGUU | UUGmUmAmGmCUC | |
| | | | GCUACAAUA | CCmUmGmAmAmAm | |
| | | | AGGCCGUCG | CmCGUUmGmCUAm | |
| | | | AAAGAUGUG | CAAU*AAGmGmCC | |
| | | | CCGCAACGCU | mGmUmCmGmAmA | |
| | | | CUGCCUUCUG | mAmGmAmUGUGCm | |
| | | | GCAUCGUU | CGmCAAmCGCUCU | |
| | | | | mGmCCmUmUmCmU | |
| | | | | GGCAUCG*mU*mU | |
| G028802 | 114 | AGAUCACCCA | AGAUCACCCA | mA*mG*mA*mUmCA | chr6: 31356262- |
| | | GCGCAAGUG | GCGCAAGUG | CmCmCAmGCmGCA | 31356286 |
| | | GGAGG | GGAGGGUUG | AGmUGGGmAGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028803 | 115 | CAGAUCACCC | CAGAUCACCC | mC*mA*mG*mAmUC | chr6: 31356263- |
| | | AGCGCAAGU | AGCGCAAGU | AmCmCCmAGmCGC | 31356287 |
| | | GGGAG | GGGAGGUUG | AAmGUGGmGAGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028804 | 116 | GCCUCCCACU | GCCUCCCACU | mG*mC*mC*mUmCC | chr6: 31356267- |
| | | UGCGCUGGG | UGCGCUGGG | CmAmCUmUGmCGC | 31356291 |
| | | UGAUC | UGAUCGUUG | UGmGGUGmAUCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028805 | 117 | CCCAAUACUC | CCCAAUACUC | mC*mC*mC*mAmAU | chr6: 31356777- |
| | | CGGCCCCUCC | CGGCCCCUCC | AmCmUCmCGmGCC | 31356801 |
| | | UGCU | UGCUGUUGU | CCmUCCUmGCUmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028806 | 118 | CACUCACCGG | CACUCACCGG | mC*mA*mC*mUmCA | chr6: 31357078- |
| | | CCCAGGUCUC | CCCAGGUCUC | CmCmGGmCCmCAG | 31357102 |
| | | GGUC | GGUCGUUGU | GUmCUCGmGUCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028807 | 119 | UAGAGCAGG | UAGAGCAGG | mU*mA*mG*mAmGC | chr6: 31356774- |
| | | AGGGGCCGG | AGGGGCCGG | AmGmGAmGGmGGC | 31356798 |
| | | AGUAUU | AGUAUUGUU | CGmGAGUmAUUmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028808 | 120 | CCGGCCCAGG | CCGGCCCAGG | mC*mC*mG*mGmCC | chr6: 31357084- |
| | | UCUCGGUCA | UCUCGGUCA | CmAmGGmUCmUCG | 31357108 |
| | | GGGCC | GGGCCGUUG | GUmCAGGmGCCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028809 | 121 | ACCACUUACA | ACCACUUACA | mA*mC*mC*mAmCU | chr6: 31354623- |
| | | CGCAGCCUGA | CGCAGCCUGA | UmAmCAmCGmCAG | 31354647 |
| | | GAGU | GAGUGUUGU | CCmUGAGmAGUmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028810 | 122 | CCACUUACAC | CCACUUACAC | mC*mC*mA*mCmUU | chr6: 31354624- |
| | | GCAGCCUGA | GCAGCCUGA | AmCmACmGCmAGC | 31354648 |
| | | GAGUA | GAGUAGUUG | CUmGAGAmGUAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028811 | 123 | UAUCUGCGG | UAUCUGCGG | mU*mA*mU*mCmUG | chr6: 31356201- |
| | | AGCCACUCCA | AGCCACUCCA | CmGmGAmGCmCAC | 31356225 |
| | | CGCAC | CGCACGUUG | UCmCACGmCACmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028812 | 124 | AGCCUGAGA | AGCCUGAGA | mA*mG*mC*mCmUG | chr6: 31354636- |
| | | GUAGCUCCCU | GUAGCUCCCU | AmGmAGmUAmGCU | 31354660 |
| | | CCUUU | CCUUUGUUG | CCmCUCCmUUUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028813 | 125 | CCUGAGUUU | CCUGAGUUU | mC*mC*mU*mGmAG | chr6: 31355460- |
| | | GGUCCUCGCC | GGUCCUCGCC | UmUmUGmGUmCCU | 31355484 |
| | | AUCCC | AUCCCGUUG | CGmCCAUmCCCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028814 | 126 | GUGUAUCUC | GUGUAUCUC | mG*mU*mG*mUmA | chr6: 31355366- |
| | | UGCUCUUCUC | UGCUCUUCUC | UCmUmCUmGCmUC | 31355390 |
| | | CAGAA | CAGAAGUUG | UUCmUCCAmGAAm | |
| | | | UAGCUCCCUG | GUUGmUmAmGmCU | |
| | | | AAACCGUUG | CCCmUmGmAmAmA | |
| | | | CUACAAUAA | mCmCGUUmGmCUA | |
| | | | GGCCGUCGA | mCAAU*AAGmGmC | |
| | | | AAGAUGUGC | CmGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028815 | 127 | CUUCUCCAGA | CUUCUCCAGA | mC*mU*mU*mCmUC | chr6: 31355379- |
| | | AGGCACCACC | AGGCACCACC | CmAmGAmAGmGCA | 31355403 |
| | | ACAG | ACAGGUUGU | CCmACCAmCAGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028816 | 128 | UACAUGGCA | UACAUGGCA | mU*mA*mC*mAmUG | chr6: 31355356- |
| | | UGUGUAUCU | UGUGUAUCU | GmCmAUmGUmGUA | 31355380 |
| | | CUGCUC | CUGCUCGUU | UCmUCUGmCUCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028817 | 129 | UAUCUCUGC | UAUCUCUGC | mU*mA*mU*mCmUC | chr6: 31355369- |
| | | UCUUCUCCAG | UCUUCUCCAG | UmGmCUmCUmUCU | 31355393 |
| | | AAGGC | AAGGCGUUG | CCmAGAAmGGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028818 | 130 | UGCUAGGAC | UGCUAGGAC | mU*mG*mC*mUmAG | chr6: 31355161- |
| | | AGCCAGGCCA | AGCCAGGCCA | GmAmCAmGCmCAG | 31355185 |
| | | GCAAC | GCAACGUUG | GCmCAGCmAACmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | | CGmCAAmCGCUCU | |
| | | | UGCCUUCUG | mGmCCmUmUmCmU | |
| | | | GCAUCGUU | GGCAUCG*mU*mU | |
| G028819 | 131 | GCUAGGACA | GCUAGGACA | mG*mC*mU*mAmGG | chr6: 31355162- |
| | | GCCAGGCCAG | GCCAGGCCAG | AmCmAGmCCmAGG | 31355186 |
| | | CAACA | CAACAGUUG | CCmAGCAmACAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028820 | 132 | GCUCCGAUG | GCUCCGAUG | mG*mC*mU*mCmCG | chr6: 31355144- |
| | | ACCACAACUG | ACCACAACUG | AmUmGAmCCmACA | 31355168 |
| | | CUAGG | CUAGGGUUG | ACmUGCUmAGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028821 | 133 | UCGUGGGCA | UCGUGGGCA | mU*mC*mG*mUmGG | chr6: 31355166- |
| | | UUGUUGCUG | UUGUUGCUG | GmCmAUmUGmUUG | 31355190 |
| | | GCCUGG | GCCUGGGUU | CUmGGCCmUGGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028822 | 134 | GAUGACCAC | GAUGACCAC | mG*mA*mU*mGmAC | chr6: 31355149- |
| | | AACUGCUAG | AACUGCUAG | CmAmCAmACmUGC | 31355173 |
| | | GACAGC | GACAGCGUU | UAmGGACmAGCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028823 | 135 | GUCCUCGUUC | GUCCUCGUUC | mG*mU*mC*mCmUC | chr6: 31356326- |
| | | AGGGCGAUG | AGGGCGAUG | GmUmUCmAGmGGC | 31356350 |
| | | UAAUC | UAAUCGUUG | GAmUGUAmAUCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028824 | 136 | CGCAGCCUGA | CGCAGCCUGA | mC*mG*mC*mAmGC | chr6: 31354633- |
| | | GAGUAGCUC | GAGUAGCUC | CmUmGAmGAmGUA | 31354657 |
| | | CCUCC | CCUCCGUUGU | GCmUCCCmUCCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028825 | 137 | GGUCAGUGU | GGUCAGUGU | mG*mG*mU*mCmAG | chr6: 31355491- |
| | | GAUCUCCGCA | GAUCUCCGCA | UmGmUGmAUmCUC | 31355515 |
| | | GGGUA | GGGUAGUUG | CGmCAGGmGUAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028826 | 138 | GUCAGUGUG | GUCAGUGUG | mG*mU*mC*mAmGU | chr6: 31355492- |
| | | AUCUCCGCAG | AUCUCCGCAG | GmUmGAmUCmUCC | 31355516 |
| | | GGUAG | GGUAGGUUG | GCmAGGGmUAGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028827 | 139 | UACAUCGCCC | UACAUCGCCC | mU*mA*mC*mAmUC | chr6: 31356317- |
| | | UGAACGAGG | UGAACGAGG | GmCmCCmUGmAAC | 31356341 |
| | | ACCUG | ACCUGGUUG | GAmGGACmCUGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028828 | 140 | GGCUCCCACU | GGCUCCCACU | mG*mG*mC*mUmCC | chr6: 31356928- |
| | | CCAUGAGGU | CCAUGAGGU | CmAmCUmCCmAUG | 31356952 |
| | | AUUUC | AUUUCGUUG | AGmGUAUmUUCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028829 | 141 | UCUUCUCCAG | UCUUCUCCAG | mU*mC*mU*mUmCU | chr6: 31355378- |
| | | AAGGCACCAC | AAGGCACCAC | CmCmAGmAAmGGC | 31355402 |
| | | CACA | CACAGUUGU | ACmCACCmACAmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028830 | 142 | AGCAUGAGG | AGCAUGAGG | mA*mG*mC*mAmUG | chr6: 31355325- |
| | | GGCUGCCGA | GGCUGCCGA | AmGmGGmGCmUGC | 31355349 |
| | | AGCCCC | AGCCCCGUUG | CGmAAGCmCCCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028831 | 143 | AGACCAGACC | AGACCAGACC | mA*mG*mA*mCmCA | chr6: 31355415- |
| | | AGCAGGAGA | AGCAGGAGA | GmAmCCmAGmCAG | 31355439 |
| | | UAGAA | UAGAAGUUG | GAmGAUAmGAAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028832 | 144 | AAGGUUCUA | AAGGUUCUA | mA*mA*mG*mGmU | chr6: 31355417- |
| | | UCUCCUGCUG | UCUCCUGCUG | UCmUmAUmCUmCC | 31355441 |
| | | GUCUG | GUCUGGUUG | UGCmUGGUmCUGm | |
| | | | UAGCUCCCUG | GUUGmUmAmGmCU | |
| | | | AAACCGUUG | CCCmUmGmAmAmA | |
| | | | CUACAAUAA | mCmCGUUmGmCUA | |
| | | | GGCCGUCGA | mCAAU*AAGmGmC | |
| | | | AAGAUGUGC | CmGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028833 | 145 | GUGGAGACC | GUGGAGACC | mG*mU*mG*mGmA | chr6: 31355419- |
| | | AGACCAGCA | AGACCAGCA | GAmCmCAmGAmCC | 31355443 |
| | | GGAGAU | GGAGAUGUU | AGCmAGGAmGAUm | |
| | | | GUAGCUCCCU | GUUGmUmAmGmCU | |
| | | | GAAACCGUU | CCCmUmGmAmAmA | |
| | | | GCUACAAUA | mCmCGUUmGmCUA | |
| | | | AGGCCGUCG | mCAAU*AAGmGmC | |
| | | | AAAGAUGUG | CmGmUmCmGmAmA | |
| | | | CCGCAACGCU | mAmGmAmUGUGCm | |
| | | | | CGmCAAmCGCUCU | |
| | | | CUGCCUUCUG | mGmCCmUmUmCmU | |
| | | | GCAUCGUU | GGCAUCG*mU*mU | |
| G028834 | 146 | GCAAGGAUU | GCAAGGAUU | mG*mC*mA*mAmGG | chr6: 31356325- |
| | | ACAUCGCCCU | ACAUCGCCCU | AmUmUAmCAmUCG | 31356349 |
| | | GAACG | GAACGGUUG | CCmCUGAmACGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028835 | 147 | ACCAUGAGG | ACCAUGAGG | mA*mC*mC*mAmUG | chr6: 31355523- |
| | | CCACCCUGAG | CCACCCUGAG | AmGmGCmCAmCCC | 31355547 |
| | | GUGCU | GUGCUGUUG | UGmAGGUmGCUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028836 | 148 | GACCAUGAG | GACCAUGAG | mG*mA*mC*mCmAU | chr6: 31355524- |
| | | GCCACCCUGA | GCCACCCUGA | GmAmGGmCCmACC | 31355548 |
| | | GGUGC | GGUGCGUUG | CUmGAGGmUGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028837 | 149 | ACCACCCCAU | ACCACCCCAU | mA*mC*mC*mAmCC | chr6: 31355538- |
| | | CUCUGACCAU | CUCUGACCAU | CmCmAUmCUmCUG | 31355562 |
| | | GAGG | GAGGGUUGU | ACmCAUGmAGGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028838 | 150 | CACCACCCCA | CACCACCCCA | mC*mA*mC*mCmAC | chr6: 31355539- |
| | | UCUCUGACCA | UCUCUGACCA | CmCmCAmUCmUCU | 31355563 |
| | | UGAG | UGAGGUUGU | GAmCCAUmGAGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028839 | 151 | AUGCCCGCGG | AUGCCCGCGG | mA*mU*mG*mCmCC | chr6: 31356374- |
| | | AGGAGGCGC | AGGAGGCGC | GmCmGGmAGmGAG | 31356398 |
| | | CCGUC | CCGUCGUUG | GCmGCCCmGUCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028840 | 152 | GAGGCGCCCG | GAGGCGCCCG | mG*mA*mG*mGmCG | chr6: 31356386- |
| | | UCCGGCCCCA | UCCGGCCCCA | CmCmCGmUCmCGG | 31356410 |
| | | CGUC | CGUCGUUGU | CCmCCACmGUCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028841 | 153 | GCUGCGACG | GCUGCGACG | mG*mC*mU*mGmCG | chr6: 31356385- |
| | | UGGGGCCGG | UGGGGCCGG | AmCmGUmGGmGGC | 31356409 |
| | | ACGGGC | ACGGGCGUU | CGmGACGmGGCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028842 | 154 | GCGACGUGG | GCGACGUGG | mG*mC*mG*mAmCG | chr6: 31356382- |
| | | GGCCGGACG | GGCCGGACG | UmGmGGmGCmCGG | 31356406 |
| | | GGCGCC | GGCGCCGUU | ACmGGGCmGCCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028843 | 155 | CGUGAGGUU | CGUGAGGUU | mC*mG*mU*mGmAG | chr6: 31356830- |
| | | CGACAGCGAC | CGACAGCGAC | GmUmUCmGAmCAG | 31356854 |
| | | GCCGC | GCCGCGUUG | CGmACGCmCGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028931 | 156 | UCCUUUUCCA | UCCUUUUCCA | mU*mC*mC*mUmUU | chr6: 31354654- |
| | | CCUGUGGGA | CCUGUGGGA | UmCmCAmCCmUGU | 31354678 |
| | | AGAAA | AGAAAGUUG | GGmGAAGmAAAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028932 | 157 | CAGAGCGAG | CAGAGCGAG | mC*mA*mG*mAmGC | chr6: 31356670- |
| | | GCCGGUGAG | GCCGGUGAG | GmAmGGmCCmGGU | 31356694 |
| | | | UGACCCGUU | GAmGUGAmCCCmG | |
| | | | GUAGCUCCCU | UUGmUmAmGmCUC | |
| | | | GAAACCGUU | CCmUmGmAmAmAm | |
| | | UGACCC | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G032787 | 158 | GGUGUAGAA | GGUGUAGAA | mG*mG*mU*mGmU | chr6: 31356928- |
| | | AUACCUCAU | AUACCUCAU | AGmAmAAmUAmCC | 31356952 |
| | | GGAGUG | GGAGUGGUU | UCAmUGGAmGUGm | |
| | | | GUAGCUCCCU | GUUGmUmAmGmCU | |
| | | | GAAACCGUU | CCCmUmGmAmAmA | |
| | | | GCUACAAUA | mCmCGUUmGmCUA | |
| | | | AGGCCGUCG | mCAAU*AAGmGmC | |
| | | | AAAGAUGUG | CmGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G032788 | 159 | AGACCCUGGC | AGACCCUGGC | mA*mG*mA*mCmCC | chr6: 31356437- |
| | | CCCGGCCCCG | CCCGGCCCCG | UmGmGCmCCmCGG | 31356461 |
| | | CGGU | CGGUGUUGU | CCmCCGCmGGUmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G032789 | 160 | GAGACCCUG | GAGACCCUG | mG*mA*mG*mAmCC | chr6: 31356436- |
| | | GCCCCGGCCC | GCCCCGGCCC | CmUmGGmCCmCCG | 31356460 |
| | | CGCGG | CGCGGGUUG | GCmCCCGmCGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | | CGmCAAmCGCUCU | |
| | | | UGCCUUCUG | mGmCCmUmUmCmU | |
| | | | GCAUCGUU | GGCAUCG*mU*mU | |
| G032790 | 161 | CUCACCGGCC | CUCACCGGCC | mC*mU*mC*mAmCC | chr6: 31356682- |
| | | UCGCUCUGG | UCGCUCUGG | GmGmCCmUCmGCU | 31356706 |
| | | UUGUA | UUGUAGUUG | CUmGGUUmGUAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032791 | 162 | CUGCGCGGCU | CUGCGCGGCU | mC*mU*mG*mCmGC | chr6: 31356694- |
| | | ACUACAACCA | ACUACAACCA | GmGmCUmACmUAC | 31356718 |
| | | GAGC | GAGCGUUGU | AAmCCAGmAGCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G032792 | 163 | CAAACUCAG | CAAACUCAG | mC*mA*mA*mAmCU | chr6: 31355446- |
| | | GACACUGAG | GACACUGAG | CmAmGGmACmACU | 31355470 |
| | | CUUGUG | CUUGUGGUU | GAmGCUUmGUGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G032793 | 164 | UCAGGACAC | UCAGGACAC | mU*mC*mA*mGmGA | chr6: 31355441- |
| | | UGAGCUUGU | UGAGCUUGU | CmAmCUmGAmGCU | 31355465 |
| | | GGAGAC | GGAGACGUU | UGmUGGAmGACmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G032794 | 165 | UCUGGGAAA | UCUGGGAAA | mU*mC*mU*mGmGG | chr6: 31355222- |
| | | GGAGGGGAA | GGAGGGGAA | AmAmAGmGAmGGG | 31355246 |
| | | GAUGAG | GAUGAGGUU | GAmAGAUmGAGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G032795 | 166 | CUCUGGGAA | CUCUGGGAA | mC*mU*mC*mUmGG | chr6: 31355221- |
| | | AGGAGGGGA | AGGAGGGGA | GmAmAAmGGmAGG | 31355245 |
| | | AGAUGA | AGAUGAGUU | GGmAAGAmUGAmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G032796 | 167 | ACCCAGUUCG | ACCCAGUUCG | mA*mC*mC*mCmAG | chr6: 31356844- |
| | | UGAGGUUCG | UGAGGUUCG | UmUmCGmUGmAGG | 31356868 |
| | | ACAGC | ACAGCGUUG | UUmCGACmAGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032797 | 168 | CGCUGCCUGG | CGCUGCCUGG | mC*mG*mC*mUmGC | chr6: 31354520- |
| | | AGUAGAACA | AGUAGAACA | CmUmGGmAGmUAG | 31354544 |
| | | AAAAC | AAAACGUUG | AAmCAAAmAACmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032798 | 169 | CUGGAGGGU | CUGGAGGGU | mC*mU*mG*mGmAG | chr6: 31356425- |
| | | GUGAGACCC | GUGAGACCC | GmGmUGmUGmAGA | 31356449 |
| | | UGGCCC | UGGCCCGUU | CCmCUGGmCCCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G032799 | 170 | GCCCAGGUCU | GCCCAGGUCU | mG*mC*mC*mCmAG | chr6: 31357087- |
| | | CGGUCAGGG | CGGUCAGGG | GmUmCUmCGmGUC | 31357111 |
| | | CCAGG | CCAGGGUUG | AGmGGCCmAGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032800 | 171 | CUGUGUGUU | CUGUGUGUU | mC*mU*mG*mUmGU | chr6: 31356763- |
| | | CCGGUCCCAA | CCGGUCCCAA | GmUmUCmCGmGUC | 31356787 |
| | | UACUC | UACUCGUUG | CCmAAUAmCUCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032801 | 172 | UCUGUGUGU | UCUGUGUGU | mU*mC*mU*mGmUG | chr6: 31356762- |
| | | UCCGGUCCCA | UCCGGUCCCA | UmGmUUmCCmGGU | 31356786 |
| | | AUACU | AUACUGUUG | CCmCAAUmACUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032802 | 173 | UGUGUGUUC | UGUGUGUUC | mU*mG*mU*mGmU | chr6: 31356764- |
| | | CGGUCCCAAU | CGGUCCCAAU | GUmUmCCmGGmUC | 31356788 |
| | | ACUCC | ACUCCGUUG | CCAmAUACmUCCm | |
| | | | UAGCUCCCUG | GUUGmUmAmGmCU | |
| | | | AAACCGUUG | CCCmUmGmAmAmA | |
| | | | CUACAAUAA | mCmCGUUmGmCUA | |
| | | | GGCCGUCGA | mCAAU*AAGmGmC | |
| | | | AAGAUGUGC | CmGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032803 | 174 | CGAACCGUCC | CGAACCGUCC | mC*mG*mA*mAmCC | chr6: 31357116- |
| | | UCCUGCUGCU | UCCUGCUGCU | GmUmCCmUCmCUG | 31357140 |
| | | CUCG | CUCGGUUGU | CUmGCUCmUCGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G032804 | 175 | UCUGGAGGG | UCUGGAGGG | mU*mC*mU*mGmGA | chr6: 31356424- |
| | | UGUGAGACC | UGUGAGACC | GmGmGUmGUmGAG | 31356448 |
| | | CUGGCC | CUGGCCGUU | ACmCCUGmGCCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | | CGmCAAmCGCUCU | |
| | | | CUGCCUUCUG | mGmCCmUmUmCmU | |
| | | | GCAUCGUU | GGCAUCG*mU*mU | |
| G032805 | 176 | CCCAGAGCCG | CCCAGAGCCG | mC*mC*mC*mAmGA | chr6: 31355204- |
| | | UCUUCCCAGU | UCUUCCCAGU | GmCmCGmUCmUUC | 31355228 |
| | | CCAC | CCACGUUGU | CCmAGUCmCACmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G032806 | 177 | UCCCAGAGCC | UCCCAGAGCC | mU*mC*mC*mCmAG | chr6: 31355205- |
| | | GUCUUCCCAG | GUCUUCCCAG | AmGmCCmGUmCUU | 31355229 |
| | | UCCA | UCCAGUUGU | CCmCAGUmCCAmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G032807 | 178 | CCAGAGCCGU | CCAGAGCCGU | mC*mC*mA*mGmAG | chr6: 31355203- |
| | | CUUCCCAGUC | CUUCCCAGUC | CmCmGUmCUmUCC | 31355227 |
| | | CACC | CACCGUUGU | CAmGUCCmACCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G032808 | 179 | GUGUUCCGG | GUGUUCCGG | mG*mU*mG*mUmUC | chr6: 31356767- |
| | | UCCCAAUACU | UCCCAAUACU | CmGmGUmCCmCAA | 31356791 |
| | | CCGGC | CCGGCGUUG | UAmCUCCmGGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032809 | 180 | UGGAGGGUG | UGGAGGGUG | mU*mG*mG*mAmG | chr6: 31356426- |
| | | UGAGACCCU | UGAGACCCU | GGmUmGUmGAmGA | 31356450 |
| | | GGCCCC | GGCCCCGUUG | CCCmUGGCmCCCm | |
| | | | UAGCUCCCUG | GUUGmUmAmGmCU | |
| | | | AAACCGUUG | CCCmUmGmAmAmA | |
| | | | CUACAAUAA | mCmCGUUmGmCUA | |
| | | | GGCCGUCGA | mCAAU*AAGmGmC | |
| | | | AAGAUGUGC | CmGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032810 | 181 | CCGGCCCCUC | CCGGCCCCUC | mC*mC*mG*mGmCC | chr6: 31356786- |
| | | CUGCUCUAUC | CUGCUCUAUC | CmCmUCmCUmGCU | 31356810 |
| | | CACG | CACGGUUGU | CUmAUCCmACGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G032811 | 182 | UCCCACUUGC | UCCCACUUGC | mU*mC*mC*mCmAC | chr6: 31356270- |
| | | GCUGGGUGA | GCUGGGUGA | UmUmGCmGCmUGG | 31356294 |
| | | UCUGA | UCUGAGUUG | GUmGAUCmUGAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G032812 | 183 | CACAGACUG | CACAGACUG | mC*mA*mC*mAmGA | chr6: 31356723- |
| | | ACCGAGAGA | ACCGAGAGA | CmUmGAmCCmGAG | 31356747 |
| | | GCCUGC | GCCUGCGUU | AGmAGCCmUGCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G032813 | 184 | UGCUGGUCA | UGCUGGUCA | mU*mG*mC*mUmGG | chr6: 31357133- |
| | | UGGCGCCCCG | UGGCGCCCCG | UmCmAUmGGmCGC | 31357157 |
| | | AACCG | AACCGGUUG | CCmCGAAmCCGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028919 | 185 | CACAUCAGA | CACAUCAGA | mC*mA*mC*mAmUC | chr6: 31354497- |
| | | GCCCUGGGCA | GCCCUGGGCA | AmGmAGmCCmCUG | 31354521 |
| | | CUGUC | CUGUCGUUG | GGmCACUmGUCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |

*The guide sequence disclosed in this Table may be unmodified, modified with the exemplary
modification pattern shown in the Table, or modified with a different modification pattern
disclosed herein or available in the art.

TABLE 3A
Additional Exemplary N. meningitidis (Nme) HLA-B guide RNAs

| | | Exemplary Guide RNA Modified Sequence |
|---|---|---|
| Guide ID | Guide Sequence | (SEQ ID NOs: 2186-2191) |
| G034206 | CAAACUCAG | mC*mA*mA*mAmCUCmAmGGmACmACUGAmGCUUmGUGmGUUGm |
| | GACACUGAG | UmAmGmCUCCCmUmGmAmAmAmCmCGUUmGmCUAmCAAUAAGm |
| | CUUGUG (SEQ | GmCCmGmUmCmGmAmAmAmGmAmUGUGCmCGmCAAmCGCUCUm |
| | ID NO: 163) | GmCCmUmUmCmUGGCAUCG*mU*mU (SEQ ID NO: 2186) |
| G034207 | UCAGGACAC | mU*mC*mA*mGmGACmAmCUmGAmGCUUGmUGGAmGACmGUUGm |
| | UGAGCUUGU | UmAmGmCUCCCmUmGmAmAmAmCmCGUUmGmCUAmCAAUAAGm |
| | GGAGAC (SEQ | GmCCmGmUmCmGmAmAmAmGmAmUGUGCmCGmCAAmCGCUCUm |
| | ID NO: 164) | GmCCmUmUmCmUGGCAUCG*mU*mU (SEQ ID NO: 2187) |
| G034208 | UCUGGGAAA | mU*mC*mU*mGmGGAmAmAGmGAmGGGGAmAGAUmGAGmGUUG |
| | GGAGGGGAA | mUmAmGmCUCCCmUmGmAmAmAmCmCGUUmGmCUAmCAAUAAG |
| | GAUGAG (SEQ | mGmCCmGmUmCmGmAmAmAmGmAmUGUGCmCGmCAAmCGCUCU |
| | ID NO: 165) | mGmCCmUmUmCmUGGCAUCG*mU*mU (SEQ ID NO: 2188) |
| G034209 | CUCUGGGAA | mC*mU*mC*mUmGGGmAmAAmGGmAGGGGmAAGAmUGAmGUUGm |
| | AGGAGGGGA | UmAmGmCUCCCmUmGmAmAmAmCmCGUUmGmCUAmCAAUAAGm |
| | AGAUGA (SEQ | GmCCmGmUmCmGmAmAmAmGmAmUGUGCmCGmCAAmCGCUCUm |
| | ID NO: 166) | GmCCmUmUmCmUGGCAUCG*mU*mU (SEQ ID NO: 2189) |
| G034210 | CUGGAGGGU | mC*mU*mG*mGmAGGmGmUGmUGmAGACCmCUGGmCCCmGUUGm |
| | GUGAGACCC | UmAmGmCUCCCmUmGmAmAmAmCmCGUUmGmCUAmCAAUAAGm |
| | UGGCCC (SEQ | GmCCmGmUmCmGmAmAmAmGmAmUGUGCmCGmCAAmCGCUCUm |
| | ID NO: 169) | GmCCmUmUmCmUGGCAUCG*mU*mU (SEQ ID NO: 2190) |
| G034211 | UCCCAGAGCC | mU*mC*mC*mCmAGAmGmCCmGUmCUUCCmCAGUmCCAmGUUGm |
| | GUCUUCCCAG | UmAmGmCUCCCmUmGmAmAmAmCmCGUUmGmCUAmCAAUAAGm |
| | UCCA (SEQ ID | GmCCmGmUmCmGmAmAmAmGmAmUGUGCmCGmCAAmCGCUCUm |
| | NO: 177) | GmCCmUmUmCmUGGCAUCG*mU*mU (SEQ ID NO: 2191) |

*The guide sequence disclosed in this Table may be unmodified, modified with
the exemplary modification pattern shown in the Table, or modified with a
different modification pattern disclosed herein or available in the art.

In some embodiments, the HLA-B gRNA comprises a guide sequence selected from any one of SEQ ID NOs: 1-91. In some embodiments, the HLA-B gRNA comprises a guide sequence selected from any one of SEQ ID NOs: 3, 13, 18, 32, 36, 39, 48-56, 58, 64-71, 73-73, 80-82, 86, and 88-91. In some embodiments, the HLA-B gRNA comprises a guide sequence selected from any one of SEQ ID NOs: 3, 13, 36, 39, 49-56, 64-71, 74, 80-82, 88, and 90-91. In some embodiments, the HLA-B gRNA comprises a guide sequence selected from any one of SEQ ID NOs: 13, 39, 49, 52, 65, 74, 82, and 91. In some embodiments, the HLA-B gRNA comprises a guide sequence selected from any one of SEQ ID NOs: 3, 39, and 49-52. In some embodiments, the HLA-B gRNA comprises a guide sequence selected from any one of SEQ ID NOs: 3, 36, 39, 49, 50, 51, and 52. In some embodiments, the HLA-B gRNA comprises a guide sequence selected from any one of SEQ ID NOs: 39, 49, and 52. In some embodiments, the HLA-B gRNA comprises a guide sequence selected from any one of SEQ ID NOs: 49, 52-54, 55, 56, 64, 65, 67-71, 73-74, 80-82, and 90. In some embodiments, the HLA-B gRNA comprises a guide sequence selected from any one of SEQ ID NOs: 49, 51, 74, 81, and 82. In some embodiments, the HLA-B gRNA comprises a guide sequence of SEQ ID NO: 13 or 74. In some embodiments, the HLA-B gRNA comprises a guide sequence of SEQ ID NO: 13. In some embodiments, the HLA-B gRNA comprises a guide sequence of SEQ ID NO: 74.

In some embodiments, the HLA-B gRNA comprises a guide sequence selected from any one of SEQ ID NOs: 101-185. In some embodiments, the HLA-B gRNA comprises a guide sequence selected from any one of SEQ ID NOs: 101, 103, 106, 107, 114, 117, 118, 125-129,137,138, 141, 143, 144, 145, 159, 160, 163, 164, 165, 166, 169, 171, 172, 173, 176, 177, 178, 179, and 180. In some embodiments, the HLA-B gRNA comprises a guide sequence selected from any one of SEQ ID NOs: 65 and 74. In some embodiments, the HLA-B gRNA comprises a guide sequence selected from any one of SEQ ID NOs: 49, 52-54, 56, 64-65, 67-71, 73-74, 80-82, 88, and 90-91. In some embodiments, the HLA-B gRNA comprises a guide sequence selected from any one of SEQ ID NOs: 74, 82, and 91.

In some embodiments, the gRNA comprises a guide sequence selected from any one of SEQ ID NOs: 1-91 or 101-185. In some embodiments, the gRNA comprises a guide sequence selected from any one of SEQ ID NOs: 101, 103, 106, 107, 117, 125-129, 137, 138, 141, 143, 144, 145, 159, 160, 163, 164, 165, 166, 169, 171, 172, 173, 176, 177, 178, 179, and 180. In some embodiments, the gRNA comprises a guide sequence selected from any one of SEQ ID NOs: 101, 103, 106, 117, 118, 125-128, 133, 137-138, 141, 143-144, 159, 163, 164, 165, 166, 169, 171, 173, 177, 178, and 180. In some embodiments, the gRNA comprises a guide sequence selected from any one of SEQ ID NOs: 101, 106, 114, 117-118, 125-128, 133, 137-138, 141, 143-144, 159, 163, 164, 165, 166, 169, 171, 173, 177, 178, and 180. In some embodiments, the gRNA comprises a guide sequence selected from any one of SEQ ID NOs: 101, 117-118, 125-128, 137-138, 144, 159, 163, 164, 165, 166, 169, 177, 178, and 180. In some embodiments, the gRNA comprises a guide sequence selected from any one of SEQ ID NOs: 101, 117, 127, 137-138, 163, 164, 165, 166, 169, and 177. In some embodiments, the gRNA comprises a guide sequence selected from any one of SEQ ID NOs: 163-166, 169, and 177. In some embodiments, the gRNA comprises a sequence selected from any one of SEQ ID NOs: 2186-2191. In some embodiments, the gRNA comprises a guide sequence comprising a sequence of SEQ ID NO: 163. In some embodiments, the gRNA comprises a guide sequence comprising a sequence of SEQ ID NO: 164. In some embodiments, the gRNA comprises a guide sequence comprising a sequence of SEQ ID NO: 165. In some embodiments, the gRNA comprises a guide sequence comprising a sequence of SEQ ID NO: 166. In some embodiments, the gRNA comprises a guide sequence comprising a sequence of SEQ ID NO: 169. In some embodiments, the gRNA comprises a guide sequence comprising a sequence of SEQ ID NO: 177.

In some embodiments, the HLA-B guide RNA comprises a guide sequence that is at least 17, 18, 19, 20, 21, 22, 23, or 24 contiguous nucleotides of a sequence selected from SEQ ID NOs: 1-91 or 101-185. In some embodiments, the HLA-B guide RNA comprises a guide sequence that is at least 95%, 90%, 85%, 80%, 75%, or 70% identical to a sequence selected from SEQ ID NOs: 1-91 or 101-185. In some embodiments, the HLA-B guide RNA comprises a guide sequence that is at least 95% identical to a sequence selected from SEQ ID NOs: 1-91 or 101-185.

In some embodiments, the HLA-B guide RNA comprises a guide sequence that comprises at least 10 contiguous nucleotides ±10 nucleotides of a genomic coordinate listed in Tables 2-3. As used herein, at least 10 contiguous nucleotides ±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Tables 2-3. For example, an HLA-B guide RNA may comprise 10 contiguous nucleotides within the genomic coordinates (a) chr6:31355348-31355368; or (b) chr6:31355390-31355414; chr6:31355417-31355441; or chr6: 31356386-31356410, including the boundary nucleotides of these ranges. In some embodiments, the HLA-B guide RNA comprises a guide sequence that is at least 17, 18, 19, or 20 contiguous nucleotides of a sequence that comprises 10 contiguous nucleotides ±10 nucleotides of a genomic coordinate listed in Table 2, or a guide sequence that is complementary to at least 17, 18, 19, 20, 21, 22, 23, or 24 contiguous nucleotides of a sequence that comprises 10 contiguous nucleotides ±10 nucleotides of a genomic coordinate listed in Table 3. In some embodiments, the HLA-B guide RNA comprises a guide sequence that is at least 95%, 90%, or 85% identical to a sequence selected from a sequence that is 17, 18, 19, or 20 contiguous nucleotides of a sequence that comprises 10 contiguous nucleotides ±10 nucleotides of a genomic coordinate listed in Table 2, or a guide sequence that is complementary to at least 17, 18, 19, 20, 21, 22, 23, or 24 contiguous nucleotides of a sequence that comprises 10 contiguous nucleotides ±10 nucleotides of a genomic coordinate listed in Table 3.

In some embodiments, the Tables 2-3 guide RNA comprises a guide sequence that comprises at least 15 contiguous nucleotides ±10 nucleotides of a genomic coordinate listed in Tables 2-3. In some embodiments, the HLA-B guide RNA comprises a guide sequence that comprises at least 20 contiguous nucleotides ±10 nucleotides of a genomic coordinate listed in Tables 2-3. In some embodiments, the HLA-B guide RNA comprises SEQ ID NO: 1. In some embodiments, the HLA-B guide RNA comprises SEQ ID NO: 2. In some embodiments, the HLA-B guide RNA comprises SEQ ID NO: 3. In some embodiments, the HLA-B guide RNA comprises SEQ ID NO: 4. In some embodiments, the HLA-B guide RNA comprises SEQ ID NO: 5. In some embodiments, the HLA-B guide RNA comprises SEQ ID NO: 6. In some embodiments, the HLA-B guide RNA comprises SEQ ID NO: 7. In some embodiments, the HLA-B guide RNA comprises SEQ ID NO: 8. In some embodiments, the HLA-B guide RNA comprises SEQ ID NO: 9. In some embodiments, the HLA-B guide RNA comprises SEQ ID NO: 10. In some embodiments, the HLA-A guide RNA comprises SEQ ID NO: 11. In some embodiments, the HLA-B guide RNA comprises SEQ ID NO: 12. In some embodiments, the HLA-B guide RNA comprises SEQ ID NO: 13. In some embodiments, the HLA-B guide RNA comprises SEQ ID NO: 14. In some embodiments, the HLA-B guide RNA comprises SEQ ID NO: 15. In some embodiments, the HLA-B guide RNA comprises SEQ ID NO: 16. In some embodiments, the HLA-B guide RNA comprises SEQ ID NO: 17. In some embodiments, the HLA-B guide RNA comprises SEQ ID NO: 18. In some embodiments, the HLA-B guide RNA comprises SEQ ID NO: 19. In some embodiments, the HLA-B guide RNA comprises SEQ ID NO: 20. In some embodiments, the HLA-B guide RNA comprises SEQ ID NO: 21. In some embodiments, the HLA-B guide RNA comprises SEQ ID NO: 22. In some embodiments, the HLA-B guide RNA comprises SEQ ID NO: 23. In some embodiments, the HLA-B guide RNA comprises SEQ ID NO: 24. In some embodiments, the HLA-B guide RNA comprises SEQ ID NO: 25. In some embodiments, the HLA-B guide RNA comprises SEQ ID NO: 26. In some embodiments, the HLA-B guide RNA comprises SEQ ID NO: 27. In some embodiments, the HLA-B guide RNA comprises SEQ ID NO: 28. In some embodiments, the HLA-B guide RNA comprises SEQ ID NO: 29. In some embodiments, the HLA-B guide RNA comprises SEQ ID NO: 30. In some embodiments, the HLA-B guide RNA comprises SEQ ID NO: 31. In some embodiments, the HLA-B guide RNA comprises SEQ ID NO: 32. In some embodiments, the HLA-B guide RNA comprises SEQ ID NO: 33. In some embodiments, the HLA-B guide RNA comprises SEQ ID NO: 34. In some embodiments, the HLA-B guide RNA comprises SEQ ID NO: 35. In some embodiments, the HLA-B guide RNA comprises SEQ ID NO: 36. In some embodiments, the HLA-B guide RNA comprises SEQ ID NO: 37. In some embodiments, the HLA-B guide RNA comprises SEQ ID NO: 38. In some embodiments, the HLA-B guide RNA comprises SEQ ID NO: 39. In some embodiments, the HLA-B guide RNA comprises SEQ ID NO: 40. In some embodiments, the HLA-B guide RNA comprises SEQ ID NO: 41. In some embodiments, the HLA-B guide RNA comprises SEQ ID NO: 42. In some embodiments, the HLA-B guide RNA comprises SEQ ID NO: 43. In some embodiments, the HLA-B guide RNA comprises SEQ ID NO: 44. In some embodiments, the HLA-B guide RNA comprises SEQ ID NO: 45. In some embodiments, the HLA-B guide RNA comprises SEQ ID NO: 46. In some embodiments, the HLA-B guide RNA comprises SEQ ID NO: 47. In some embodiments, the HLA-B guide RNA comprises SEQ ID NO: 48. In some embodiments, the HLA-B guide RNA comprises SEQ ID NO: 49. In some embodiments, the HLA-B guide RNA comprises SEQ ID NO: 50. In some embodiments, the HLA-B guide RNA comprises SEQ ID NO: 51. In some embodiments, the HLA-B guide RNA comprises SEQ ID NO: 52. In some embodiments, the HLA-B guide RNA comprises SEQ ID NO: 53. In some embodiments, the HLA-B guide RNA comprises SEQ ID NO: 54. In some embodiments, the HLA-B guide RNA comprises SEQ ID NO: 55. In some embodiments, the HLA-B guide RNA comprises SEQ ID NO: 56. In some embodiments, the HLA-B guide RNA comprises SEQ ID NO: 57. In some embodiments, the HLA-B guide RNA comprises SEQ ID NO: 58. In some embodiments, the HLA-B guide RNA comprises SEQ ID NO: 59. In some embodiments, the HLA-B guide RNA comprises SEQ ID NO: 60. In some embodiments, the HLA-B guide RNA comprises SEQ ID NO: 61. In some embodiments, the HLA-B guide RNA comprises SEQ ID NO: 62. In some embodiments, the HLA-B guide RNA comprises SEQ ID NO: 63. In some embodiments, the HLA-B guide RNA comprises SEQ ID NO: 64. In some embodiments, the HLA-B guide RNA comprises SEQ ID NO: 65. In some embodiments, the HLA-B guide RNA comprises SEQ ID NO: 66. In some embodiments, the HLA-B guide RNA comprises SEQ ID NO: 67. In some embodiments, the HLA-B guide RNA comprises SEQ ID NO: 68. In some embodiments, the HLA-B guide RNA comprises SEQ ID NO: 69. In some embodiments, the HLA-B guide RNA comprises SEQ ID NO: 70. In some embodiments, the HLA-B guide RNA comprises SEQ ID NO: 71. In some embodiments, the HLA-B guide RNA comprises SEQ ID NO: 72. In some embodiments, the HLA-B guide RNA comprises SEQ ID NO: 73. In some embodiments, the HLA-B guide RNA comprises SEQ ID NO: 74. In some embodiments, the HLA-B guide RNA comprises SEQ ID NO: 75. In some embodiments, the HLA-B guide RNA comprises SEQ ID NO: 76. In some embodiments, the HLA-B guide RNA comprises SEQ ID NO: 77. In some embodiments, the HLA-B guide RNA comprises SEQ ID NO: 78. In some embodiments, the HLA-B guide RNA comprises SEQ ID NO: 79. In some embodiments, the HLA-B guide RNA comprises SEQ ID NO: 80. In some embodiments, the HLA-B guide RNA comprises SEQ ID NO: 81. In some embodiments, the HLA-B guide RNA comprises SEQ ID NO: 82. In some embodiments, the HLA-B guide RNA comprises SEQ ID NO: 83. In some embodiments, the HLA-B guide RNA comprises SEQ ID NO: 84. In some embodiments, the HLA-B guide RNA comprises SEQ ID NO: 85. In some embodiments, the HLA-B guide RNA comprises SEQ ID NO: 86. In some embodiments, the HLA-B guide RNA comprises SEQ ID NO: 87. In some embodiments, the HLA-B guide RNA comprises SEQ ID NO: 88. In some embodiments, the HLA-B guide RNA comprises SEQ ID NO: 89. In some embodiments, the HLA-B guide RNA comprises SEQ ID NO: 90. In some embodiments, the HLA-B guide RNA comprises SEQ ID NO: 91.

In some embodiments, the HLA-B guide RNA comprises SEQ ID NO: 101. In some embodiments, the HLA-B guide RNA comprises SEQ ID NO: 102. In some embodiments, the HLA-B guide RNA comprises SEQ ID NO: 103. In some embodiments, the HLA-B guide RNA comprises SEQ ID NO: 104. In some embodiments, the HLA-B guide RNA comprises SEQ ID NO: 105. In some embodiments, the HLA-B guide RNA comprises SEQ ID NO: 106. In some embodiments, the HLA-B guide RNA comprises SEQ ID NO: 107. In some embodiments, the HLA-B guide RNA comprises SEQ ID NO: 108. In some embodiments, the HLA-B guide RNA comprises SEQ ID NO: 109. In some embodiments, the HLA-B guide RNA comprises SEQ ID NO: 110. In some embodiments, the HLA-B guide RNA comprises SEQ ID NO: 111. In some embodiments, the HLA-B guide RNA comprises SEQ ID NO: 112. In some embodiments, the HLA-B guide RNA comprises SEQ ID NO: 113. In some embodiments, the HLA-B guide RNA comprises SEQ ID NO: 114. In some embodiments, the HLA-B guide RNA comprises SEQ ID NO: 115. In some embodiments, the HLA-B guide RNA comprises SEQ ID NO: 116. In some embodiments, the HLA-B guide RNA comprises SEQ ID NO: 117. In some embodiments, the HLA-B guide RNA comprises SEQ ID NO: 118. In some embodiments, the HLA-B guide RNA comprises SEQ ID NO: 119. In some embodiments, the HLA-B guide RNA comprises SEQ ID NO: 120. In some embodiments, the HLA-B guide RNA comprises SEQ ID NO: 121. In some embodiments, the HLA-B guide RNA comprises SEQ ID NO: 122. In some embodiments, the HLA-B guide RNA comprises SEQ ID NO: 123. In some embodiments, the HLA-B guide RNA comprises SEQ ID NO: 124. In some embodiments, the HLA-B guide RNA comprises SEQ ID NO: 125. In some embodiments, the HLA-B guide RNA comprises SEQ ID NO: 126. In some embodiments, the HLA-B guide RNA comprises SEQ ID NO: 127. In some embodiments, the HLA-B guide RNA comprises SEQ ID NO: 128. In some embodiments, the HLA-B guide RNA comprises SEQ ID NO: 129. In some embodiments, the HLA-B guide RNA comprises SEQ ID NO: 130. In some embodiments, the HLA-B guide RNA comprises SEQ ID NO: 131. In some embodiments, the HLA-B guide RNA comprises SEQ ID NO: 132. In some embodiments, the HLA-B guide RNA comprises SEQ ID NO: 133. In some embodiments, the HLA-B guide RNA comprises SEQ ID NO: 134. In some embodiments, the HLA-B guide RNA comprises SEQ ID NO: 135. In some embodiments, the HLA-B guide RNA comprises SEQ ID NO: 136. In some embodiments, the HLA-B guide RNA comprises SEQ ID NO: 137. In some embodiments, the HLA-B guide RNA comprises SEQ ID NO: 138. In some embodiments, the HLA-B guide RNA comprises SEQ ID NO: 139. In some embodiments, the HLA-B guide RNA comprises SEQ ID NO: 140. In some embodiments, the HLA-B guide RNA comprises SEQ ID NO: 141. In some embodiments, the HLA-B guide RNA comprises SEQ ID NO: 142. In some embodiments, the HLA-B guide RNA comprises SEQ ID NO: 143. In some embodiments, the HLA-B guide RNA comprises SEQ ID NO: 144. In some embodiments, the HLA-B guide RNA comprises SEQ ID NO: 145. In some embodiments, the HLA-B guide RNA comprises SEQ ID NO: 146. In some embodiments, the HLA-B guide RNA comprises SEQ ID NO: 147. In some embodiments, the HLA-B guide RNA comprises SEQ ID NO: 148. In some embodiments, the HLA-B guide RNA comprises SEQ ID NO: 149. In some embodiments, the HLA-B guide RNA comprises SEQ ID NO: 150. In some embodiments, the HLA-B guide RNA comprises SEQ ID NO: 151. In some embodiments, the HLA-B guide RNA comprises SEQ ID NO: 152. In some embodiments, the HLA-B guide RNA comprises SEQ ID NO: 153. In some embodiments, the HLA-B guide RNA comprises SEQ ID NO: 154. In some embodiments, the HLA-B guide RNA comprises SEQ ID NO: 155. In some embodiments, the HLA-B guide RNA comprises SEQ ID NO: 156. In some embodiments, the HLA-B guide RNA comprises SEQ ID NO: 157. In some embodiments, the HLA-B guide RNA comprises SEQ ID NO: 158. In some embodiments, the HLA-B guide RNA comprises SEQ ID NO: 159. In some embodiments, the HLA-B guide RNA comprises SEQ ID NO: 160. In some embodiments, the HLA-B guide RNA comprises SEQ ID NO: 161. In some embodiments, the HLA-B guide RNA comprises SEQ ID NO: 162. In some embodiments, the HLA-B guide RNA comprises SEQ ID NO: 163. In some embodiments, the HLA-B guide RNA comprises SEQ ID NO: 164. In some embodiments, the HLA-B guide RNA comprises SEQ ID NO: 165. In some embodiments, the HLA-B guide RNA comprises SEQ ID NO: 166. In some embodiments, the HLA-B guide RNA comprises SEQ ID NO: 167. In some embodiments, the HLA-B guide RNA comprises SEQ ID NO: 168. In some embodiments, the HLA-B guide RNA comprises SEQ ID NO: 169. In some embodiments, the HLA-B guide RNA comprises SEQ ID NO: 170. In some embodiments, the HLA-B guide RNA comprises SEQ ID NO: 171. In some embodiments, the HLA-B guide RNA comprises SEQ ID NO: 172. In some embodiments, the HLA-B guide RNA comprises SEQ ID NO: 173. In some embodiments, the HLA-B guide RNA comprises SEQ ID NO: 174. In some embodiments, the HLA-B guide RNA comprises SEQ ID NO: 175. In some embodiments, the HLA-B guide RNA comprises SEQ ID NO: 176. In some embodiments, the HLA-B guide RNA comprises SEQ ID NO: 177. In some embodiments, the HLA-B guide RNA comprises SEQ ID NO: 178. In some embodiments, the HLA-B guide RNA comprises SEQ ID NO: 179. In some embodiments, the HLA-B guide RNA comprises SEQ ID NO: 180. In some embodiments, the HLA-B guide RNA comprises SEQ ID NO: 181. In some embodiments, the HLA-B guide RNA comprises SEQ ID NO: 182. In some embodiments, the HLA-B guide RNA comprises SEQ ID NO: 183. In some embodiments, the HLA-B guide RNA comprises SEQ ID NO: 184. In some embodiments, the HLA-B guide RNA comprises SEQ ID NO: 185. In some embodiments, the HLA-B guide RNA comprises SEQ ID NO: 2186. In some embodiments, the HLA-B guide RNA comprises SEQ ID NO: 2187. In some embodiments, the HLA-B guide RNA comprises SEQ ID NO: 2188. In some embodiments, the HLA-B guide RNA comprises SEQ ID NO: 2189. In some embodiments, the HLA-B guide RNA comprises SEQ ID NO: 2190. In some embodiments, the HLA-B guide RNA comprises SEQ ID NO: 2191.

Additional embodiments of HLA-B guide RNAs are provided herein, including e.g., exemplary modifications to the guide RNA. 2. Genetic modifications to HLA-B

In some embodiments, the methods and compositions disclosed herein genetically modify at least one nucleotide in the HLA-B gene in a cell. Genetic modifications encompass the population of modifications that results from contact with a gene editing system (e.g., the population of edits that result from Cas9 and an HLA-B guide RNA, or the population of edits that result from BC22 and an HLA-B guide RNA).

In some embodiments, the genetic modification comprises at least one nucleotide within the genomic coordinates chosen from: (a) chr6: 31355182-31355596 or (b) chr6:31355203-31356461.

In some embodiments, the genetic modification comprises at least one nucleotide within the genomic coordinates chosen from: chr6:31355182-31355202; chr6:31355348-31355368; chr6:31355145-31355165; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355347-31355367; chr6:31355432-31355452; chr6:31355340-31355360; chr6:31355410-31355430; chr6:31355414-31355434; or chr6:31355409-31355429.

In some embodiments, the genetic modification comprises at least one nucleotide within the genomic coordinates chosen from: chr6:31355182-31355202; chr6:31355349-31355369; chr6:31355348-31355368; or chr6:31355145-31355165.

In some embodiments, the genetic modification comprises at least one nucleotide within the genomic coordinates chosen from: chr6:31355348-31355368; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; or chr6:31355414-31355434.

In some embodiments, the genetic modification comprises at least one nucleotide within the genomic coordinates chosen from: chr6:31355348-31355368; chr6:31355349-31355369; chr6:31356381-31356401; chr6:31356380-31356400; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; chr6:31355410-31355430; chr6:31355414-31355434; or chr6:31355409-31355429.

In some embodiments, the genetic modification comprises at least one nucleotide within the genomic coordinates chosen from: chr6:31355348-31355368; chr6:31355349-31355369; chr6:31355192-31355212; chr6:31355347-31355367; chr6:31355340-31355360; and chr6:31355409-31355429. In some embodiments, the genetic modification comprises at least one nucleotide within the genomic coordinates chosen from: chr6:31355349-31355369 or chr6:31355348-31355368. In some embodiments, the genetic modification comprises at least one nucleotide within the genomic coordinates chosen from: chr6:31355192-31355212 or chr6:31355347-31355367. In some embodiments, the genetic modification comprises at least one nucleotide within the genomic coordinates chosen from: chr6:31355347-31355367; chr6:31355340-31355360; or chr6:31355409-31355429. In some embodiments, the genetic modification comprises at least one nucleotide within the genomic coordinates chosen from; chr6:31355348-31355368; chr6:31355145-31355165; chr6:31355347-31355367; chr6:31355432-31355452; or chr6:31355340-31355360.

In some embodiments, the genetic modification comprises at least one nucleotide within the genomic coordinates chosen from: chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

In some embodiments, the genetic modification comprises at least one nucleotide within the genomic coordinates chosen from: chr6:31356777-31356801; chr6:31355492-31355516; chr6:31355361-31355385; chr6:31355379-31355403; chr6:31355491-31355515; chr6:31355356-31355380; chr6:31355366-31355390; chr6:31355417-31355441; chr6:31357078-31357102; chr6:31355460-31355484; chr6:31355415-31355439; chr6:31355166-31355190; chr6:31355378-31355402; chr6:31355401-31355425; chr6:31356262-31356286; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

In some embodiments, the genetic modification comprises at least one nucleotide within the genomic coordinates chosen from: chr6:31356777-31356801; chr6:31355492-31355516; chr6:31355379-31355403; ch6:31355491-31355515; chr6:31355361-31355385; chr6:31355356-31355380; chr6:31355460-31355484; chr6:31357078-31357102; chr6:31355417-31355441; chr6:31355366-31355390; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

In some embodiments, the genetic modification comprises at least one nucleotide within the genomic coordinates chosen from: chr6:31356777-31356801; chr6:31355492-31355516; chr6:31355379-31355403; ch6:31355491-31355515; chr6:31355361-31355385; chr6:31355221-31355245; chr6:31355222-31355246; chr6:31355205-31355229; chr6:31355446-31355470; chr6:31356425-31356449; or chr6:31355441-31355465.

In some embodiments, the genetic modification comprises at least one nucleotide within the genomic coordinates chosen from: chr6:31355221-31355245; chr6:31355222-31355246; chr6:31355205-31355229; chr6:31355446-31355470; chr6:31356425-31356449; or chr6:31355441-31355465.

In some embodiments, the genetic modification comprises at least one nucleotide within the genomic coordinates chosen from: (a) chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; and chr6:31355409-31355429; or (b) chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

In some embodiments, the genetic modification comprises at least one nucleotide within the genomic coordinates chosen from: (a) chr6:31355348-31355368; or (b) chr6:31355390-31355414; chr6:31355417-31355441; or chr6: 31356386-31356410.

In some embodiments, the genetic modification comprises an indel, a C to T substitution, or an A to G substitution within the genomic coordinates chosen from; chr6:31355182-31355202; chr6:31355348-31355368; chr6:31355145-31355165; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355347-31355367; chr6:31355432-31355452; chr6:31355340-31355360; chr6:31355410-31355430; chr6:31355414-31355434; or chr6:31355409-31355429.

In some embodiments, the genetic modification comprises an indel, a C to T substitution, or an A to G substitution within the genomic coordinates chosen from; chr6:31355182-31355202; chr6:31355349-31355369; chr6:31355348-31355368; or chr6:31355145-31355165.

In some embodiments, the genetic modification comprises an indel, a C to T substitution, or an A to G substitution within the genomic coordinates chosen from; chr6:31355348-31355368; chr6:31355349-31355369; chr6:31356381-31356401; chr6:31356380-31356400; chr6:31355204-31355224; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; or chr6:31355414-31355434.

In some embodiments, the genetic modification comprises an indel, a C to T substitution, or an A to G substitution within the genomic coordinates chosen from; chr6:31355348-31355368; chr6:31355349-31355369; chr6:31356381-31356401; chr6:31356380-31356400; chr6:31355205-31355225; chr6:31355191-31355211; chr6:31355192-31355212; chr6:31355193-31355213; chr6:31355198-31355218; chr6:31355320-31355340; chr6:31355319-31355339; chr6:31355182-31355202; chr6:31355178-31355198; chr6:31355347-31355367; chr6:31355145-31355165; chr6:31355432-31355452; chr6:31355340-31355360; chr6:31355410-31355430; chr6:31355414-31355434; or chr6:31355409-31355429.

In some embodiments, the genetic modification comprises an indel, a C to T substitution, or an A to G substitution within the genomic coordinates chosen from; chr6:31355348-31355368, chr6:31355349-31355369, chr6:31355192-31355212, chr6:31355347-31355367, chr6:31355340-31355360, chr6:31355409-31355429. In some embodiments, the genetic modification comprises an indel, a C to T substitution, or an A to G substitution within the genomic coordinates chosen from: chr6:31355349-31355369 or chr6:31355348-31355368. In some embodiments, the genetic modification comprises an indel, a C to T substitution, or an A to G substitution within the genomic coordinates chosen from: chr6:31355192-31355212 or chr6:31355347-31355367. In some embodiments, the genetic modification comprises an indel, a C to T substitution, or an A to G substitution within the genomic coordinates chosen from: chr6:31355347-31355367; chr6:31355340-31355360; or chr6:31355409-31355429. In some embodiments, the genetic modification comprises an indel, a C to T substitution, or an A to G substitution within the genomic coordinates chosen from: chr6:31355348-31355368; chr6:31355145-31355165; chr6:31355347-31355367; chr6:31355432-31355452; or chr6:31355340-31355360.

In some embodiments, the genetic modification comprises an indel, a C to T substitution, or an A to G substitution within the genomic coordinates chosen from; chr6:31356777-31356801; chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

In some embodiments, the genetic modification comprises an indel, a C to T substitution, or an A to G substitution within the genomic coordinates chosen from; chr6:31356777-31356801; chr6:31355492-31355516; chr6:31355361-31355385; chr6:31355379-31355403; chr6:31355491-31355515; chr6:31355356-31355380; chr6:31355366-31355390; chr6:31355417-31355441; chr6:31357078-31357102; chr6:31355460-31355484; chr6:31355415-31355439; chr6:31355166-31355190; chr6:31355378-31355402; chr6:31355401-31355425; and chr6:31356262-31356286; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

In some embodiments, the genetic modification comprises an indel, a C to T substitution, or an A to G substitution within the genomic coordinates chosen from; chr6:31356777-31356801; chr6:31355492-31355516; chr6:31355379-31355403; ch6:31355491-31355515; chr6:31355361-31355385; chr6:31355356-31355380; chr6:31355460-31355484; chr6:31357078-31357102; chr6:31355417-31355441; chr6:31355366-31355390; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; or chr6:31356764-31356788.

In some embodiments, the genetic modification comprises an indel, a C to T substitution, or an A to G substitution within the genomic coordinates chosen from; chr6:31356777-31356801; chr6:31355492-31355516; chr6:31355379-31355403; ch6:31355491-31355515; chr6:31355361-31355385; chr6:31355221-31355245; chr6:31355222-31355246; chr6:31355205-31355229; chr6:31355446-31355470; chr6:31356425-31356449; or chr6:31355441-31355465.

In some embodiments, the genetic modification comprises an indel, a C to T substitution, or an A to G substitution within the genomic coordinates chosen from; chr6:31355221-31355245; chr6:31355222-31355246; chr6:31355205-31355229; chr6:31355446-31355470; chr6:31356425-31356449; or chr6:31355441-31355465.

In some embodiments, the genetic modification comprises an indel, a C to T substitution, or an A to G substitution within the genomic coordinates chosen from; chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; and chr6:31355409-31355429; or (b) chr6:31356777-31356801; chr6:31355492-31355516; chr6:31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286; chr6:31355419-31355443; chr6:31355390-31355414; chr6:31355369-31355393; chr6:31355221-31355245; chr6:31355222-31355246; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; or chr6:31356767-31356791.

In some embodiments, the genetic modification comprises an indel, a C to T substitution, or an A to G substitution within the genomic coordinates chosen from: (a) chr6:31355348-31355368; or (b) chr6:31355390-31355414; chr6:31355417-31355441; or chr6: 31356386-31356410.

In some embodiments, the modification to HLA-B comprises any one or more of an insertion, deletion, substitution, or deamination of at least one nucleotide in a target sequence. In some embodiments, the modification to HLA-B comprises an insertion of 1, 2, 3, 4 or 5 or more nucleotides in a target sequence. In some embodiments, the modification to HLA-B comprises a deletion of 1, 2, 3, 4 or 5 or more nucleotides in a target sequence. In other embodiments, the modification to HLA-B comprises an insertion of 1, 2, 3, 4, 5, 6, 7, 8, 9, 10, 15, 20 or 25 or more nucleotides in a target sequence. In other embodiments, the modification to HLA-B comprises a deletion of 1, 2, 3, 4, 5, 6, 7, 8, 9, 10, 15, 20 or 25 or more nucleotides in a target sequence. In some embodiments, the modification to HLA-B comprises an indel, which is generally defined in the art as an insertion or deletion of less than 1000 base pairs (bp). In some embodiments, the modification to HLA-B comprises an indel which results in a frameshift mutation in a target sequence. In some embodiments, the modification to HLA-B comprises a substitution of 1, 2, 3, 4, 5, 6, 7, 8, 9, 10, 15, 20 or 25 or more nucleotides in a target sequence. In some embodiments, the modification to HLA-B comprises one or more of an insertion, deletion, or substitution of nucleotides resulting from the incorporation of a template nucleic acid. In some embodiments, the modification to HLA-B comprises an insertion of a donor nucleic acid in a target sequence. In some embodiments, the modification to HLA-B is not transient.

### 3. HLA-A Guide RNAs

The methods and compositions provided herein disclose guide RNAs useful for reducing or eliminating the surface expression of HLA-A protein. In some embodiments, such guide RNAs direct an RNA-guided DNA binding agent to an HLA-A genomic target sequence and may be referred to herein as “HLA-A guide RNAs.” In some embodiments, the HLA-A guide RNA directs an RNA-guided DNA binding agent to a human HLA-A genomic target sequence. In some embodiments, the HLA-A guide RNA comprises a guide sequence selected from SEQ ID NO: 301-428, 429-462, 463-511 and 512-590. Further detailed description of the guide RNAs for reducing or eliminating the surface expression of HLA-A protein and for genetic modifications of HLA-A are provided in PCT/US2021/064930, the entire contents of which is incorporated herein by reference.

In some embodiments, a composition is provided comprising an HLA-A guide RNA described herein and an RNA-guided DNA binding agent or a nucleic acid encoding an RNA-guided DNA binding agent.

In some embodiments, a composition is provided comprising an HLA-A single-guide RNA (sgRNA) comprising a guide sequence selected from SEQ ID NO: 301-590. In some embodiments, a composition is provided comprising HLA-A sgRNA described herein and an RNA-guided DNA binding agent or a nucleic acid encoding an RNA-guided DNA binding agent.

In some embodiments, a composition is provided comprising an HLA-A dual-guide RNA (dgRNA) comprising a guide sequence selected from SEQ ID NO: 301-590. In some embodiments, a composition is provided comprising an HLA-A dgRNA described herein and an RNA-guided DNA binding agent or a nucleic acid encoding an RNA-guided DNA binding agent.

In some embodiments, the HLA-A gRNA comprises a guide sequence selected from any one of SEQ ID NOs: 301-590. Exemplary HLA-A guide sequences are shown below in Table 4 (SEQ ID NOs: 301-428), Table 5A and Table 5B (SEQ ID NOs: 429-482), and Table 6 (SEQ ID NOs:483-498, 500-511), and Table 7 (SEQ ID NOs: 512-590). In some embodiments, the HLA-A gRNA is a sgRNA comprising a sequence as shown below in Table 4 (SEQ ID NOs: 1301-1428 and 2301-2428), Table 6 (SEQ ID NOs: 1483-1498, 1500-1511, 2483-2498, 2500-2511), Table 7 (SEQ ID NOs: 1512-1590 and 2512-2590), and Table 9A (SEQ ID NOs: 3111 and 3112).

TABLE 4
Exemplary Spy HLA-A guide RNAs

| | | | | Exemplary Mod | |
|---|---|---|---|---|---|
| | | | | Sequence | |
| | | | | (four terminal U | |
| | SEQ ID | | Exemplary | residues are optional | |
| | NO to the | | Full Sequence | and may include 0, 1, | Genomic |
| | Guide | | (SEQ ID NOS: | 2, 3, 4, or more Us) | Coordinates |
| Guide ID | Sequence | Guide Sequence | 1301-1428) | (SEQ ID NOs: 2301-2428) | (hg38) |
| G018983 | 301 | UGGAGGGCC | UGGAGGGCC | mU*mG*mG*AGG | chr6: 29945290- |
| | | UGAUGUGUG | UGAUGUGUG | GCCUGAUGUGUG | 29945310 |
| | | UU | UUGUUUUAG | UUGUUUUAGAmG | (mismatch to |
| | | | AGCUAGAAA | mCmUmAmGmAm | hg38 = 2) |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018984 | 302 | GCCUGAUGU | GCCUGAUGU | mG*mC*mC*UGAU | chr6: 29945296- |
| | | GUGUUGGGU | GUGUUGGGU | GUGUGUUGGGUG | 29945316 |
| | | GU | GUGUUUUAG | UGUUUUAGAmGm | (mismatch to |
| | | | AGCUAGAAA | CmUmAmGmAmA | hg38 = 2) |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018985 | 303 | CCUGAUGUG | CCUGAUGUG | mC*mC*mU*GAUG | chr6: 29945297- |
| | | UGUUGGGUG | UGUUGGGUG | UGUGUUGGGUGU | 29945317 |
| | | UU | UUGUUUUAG | UGUUUUAGAmGm | (mismatch to |
| | | | AGCUAGAAA | CmUmAmGmAmA | hg38 = 1) |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018986 | 304 | CCCAACACCC | CCCAACACCC | mC*mC*mC*AACA | chr6: 29945300- |
| | | AACACACAUC | AACACACAUC | CCCAACACACAU | 29945320 |
| | | | GUUUUAGAG | CGUUUUAGAmGm | (mismatch to |
| | | | CUAGAAAUA | CmUmAmGmAmA | hg38 = 1) |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018965 | 305 | UCAGGAAAC | UCAGGAAAC | mU*mC*mA*GGA | chr6: 29890117- |
| | | AUGAAGAAA | AUGAAGAAA | AACAUGAAGAAA | |
| | | GC | GCGUUUUAG | GCGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | 29890137 |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G019018 | 306 | AGGCGCCUG | AGGCGCCUG | mA*mG*mG*CGCC | chr6: 29927058- |
| | | GGCCUCUCCC | GGCCUCUCCC | UGGGCCUCUCCC | 29927078 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018937 | 307 | CGGGCUGGCC | CGGGCUGGCC | mC*mG*mG*GCUG | chr6: 29934330- |
| | | UCCCACAAGG | UCCCACAAGG | GCCUCCCACAAG | 29934350 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018990 | 308 | ACGGCCAUCC | ACGGCCAUCC | mA*mC*mG*GCCA | chr6: 29942541- |
| | | UCGGCGUCU | UCGGCGUCU | UCCUCGGCGUCU | 29942561 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018991 | 309 | GACGGCCAUC | GACGGCCAUC | mG*mA*mC*GGCC | chr6: 29942542- |
| | | CUCGGCGUCU | CUCGGCGUCU | AUCCUCGGCGUC | 29942562 |
| | | | GUUUUAGAG | UGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018992 | 310 | GACGCCGAG | GACGCCGAG | mG*mA*mC*GCCG | chr6: 29942543- |
| | | GAUGGCCGU | GAUGGCCGU | AGGAUGGCCGUC | 29942563 |
| | | CA | CAGUUUUAG | AGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018993 | 311 | UGACGGCCA | UGACGGCCA | mU*mG*mA*CGGC | chr6: 29942543- |
| | | UCCUCGGCGU | UCCUCGGCGU | CAUCCUCGGCGU | 29942563 |
| | | C | CGUUUUAGA | CGUUUUAGAmGm | |
| | | | GCUAGAAAU | CmUmAmGmAmA | |
| | | | AGCAAGUUA | mAm UmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018994 | 312 | GGCGCCAUG | GGCGCCAUG | mG*mG*mC*GCCA | chr6: 29942550- |
| | | ACGGCCAUCC | ACGGCCAUCC | UGACGGCCAUCC | 29942570 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018995 | 313 | ACAGCGACGC | ACAGCGACGC | mA*mC*mA*GCGA | chr6: 29942864- |
| | | CGCGAGCCAG | CGCGAGCCAG | CGCCGCGAGCCA | 29942884 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018996 | 314 | CGACGCCGCG | CGACGCCGCG | mC*mG*mA*CGCC | chr6: 29942868- |
| | | AGCCAGAGG | AGCCAGAGG | GCGAGCCAGAGG | 29942888 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018997 | 315 | CGAGCCAGA | CGAGCCAGA | mC*mG*mA*GCCA | chr6: 29942876- |
| | | GGAUGGAGC | GGAUGGAGC | GAGGAUGGAGCC | 29942896 |
| | | CG | CGGUUUUAG | GGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018998 | 316 | CGGCUCCAUC | CGGCUCCAUC | mC*mG*mG*CUCC | chr6: 29942876- |
| | | CUCUGGCUCG | CUCUGGCUCG | AUCCUCUGGCUC | 29942896 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018999 | 317 | GAGCCAGAG | GAGCCAGAG | mG*mA*mG*CCAG | chr6: 29942877- |
| | | GAUGGAGCC | GAUGGAGCC | AGGAUGGAGCCG | 29942897 |
| | | GC | GCGUUUUAG | CGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G019000 | 318 | GCGCCCGCGG | GCGCCCGCGG | mG*mC*mG*CCCG | chr6: 29942883- |
| | | CUCCAUCCUC | CUCCAUCCUC | CGGCUCCAUCCU | 29942903 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019001 | 319 | GCCCGUCCGU | GCCCGUCCGU | mG*mC*mC*CGUC | chr6: 29943062- |
| | | GGGGGAUGA | GGGGGAUGA | CGUGGGGGAUGA | 29943082 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019002 | 320 | UCAUCCCCCA | UCAUCCCCCA | mU*mC*mA*UCCC | chr6: 29943063- |
| | | CGGACGGGCC | CGGACGGGCC | CCACGGACGGGC | 29943083 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019003 | 321 | AUCUCGGACC | AUCUCGGACC | mA*mU*mC*UCGG | chr6: 29943092- |
| | | CGGAGACUG | CGGAGACUG | ACCCGGAGACUG | 29943112 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019004 | 322 | GGGGUCCCGC | GGGGUCCCGC | mG*mG*mG*GUCC | chr6: 29943115- |
| | | GGCUUCGGG | GGCUUCGGG | CGCGGCUUCGGG | 29943135 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019005 | 323 | CUCGGGGUCC | CUCGGGGUCC | mC*mU*mC*GGGG | chr6: 29943118- |
| | | CGCGGCUUCG | CGCGGCUUCG | UCCCGCGGCUUC | 29943138 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019006 | 324 | UCUCGGGGU | UCUCGGGGU | mU*mC*mU*CGGG | chr6: 29943119- |
| | | CCCGCGGCUU | CCCGCGGCUU | GUCCCGCGGCUU | 29943139 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019007 | 325 | GUCUCGGGG | GUCUCGGGG | mG*mU*mC*UCGG | chr6: 29943120- |
| | | UCCCGCGGCU | UCCCGCGGCU | GGUCCCGCGGCU | 29943140 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019008 | 326 | GCAAGGGUC | GCAAGGGUC | mG*mC*mA*AGG | chr6: 29943126- |
| | | UCGGGGUCCC | UCGGGGUCCC | GUCUCGGGGUCC | 29943146 |
| | | G | GGUUUUAGA | CGGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019009 | 327 | GGACCCCGAG | GGACCCCGAG | mG*mG*mA*CCCC | chr6: 29943128- |
| | | ACCCUUGCCC | ACCCUUGCCC | GAGACCCUUGCC | 29943148 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019010 | 328 | GACCCCGAGA | GACCCCGAGA | mG*mA*mC*CCCG | chr6: 29943129- |
| | | CCCUUGCCCC | CCCUUGCCCC | AGACCCUUGCCC | 29943149 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019011 | 329 | CGAGACCCUU | CGAGACCCUU | mC*mG*mA*GACC | chr6: 29943134- |
| | | GCCCCGGGAG | GCCCCGGGAG | CUUGCCCCGGGA | 29943154 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019012 | 330 | CUCCCGGGGC | CUCCCGGGGC | mC*mU*mC*CCGG | chr6: 29943134- |
| | | AAGGGUCUC | AAGGGUCUC | GGCAAGGGUCUC | 29943154 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019013 | 331 | UCUCCCGGGG | UCUCCCGGGG | mU*mC*mU*CCCG | chr6: 29943135- |
| | | CAAGGGUCU | CAAGGGUCU | GGGCAAGGGUCU | 29943155 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019014 | 332 | CUCUCCCGGG | CUCUCCCGGG | mC*mU*mC*UCCC | chr6: 29943136- |
| | | GCAAGGGUC | GCAAGGGUC | GGGGCAAGGGUC | 29943156 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019015 | 333 | CCUUGCCCCG | CCUUGCCCCG | mC*mC*mU*UGCC | chr6: 29943140- |
| | | GGAGAGGCC | GGAGAGGCC | CCGGGAGAGGCC | 29943160 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019016 | 334 | CUGGGCCUCU | CUGGGCCUCU | mC*mU*mG*GGCC | chr6: 29943142- |
| | | CCCGGGGCAA | CCCGGGGCAA | UCUCCCGGGGCA | 29943162 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019017 | 335 | CCUGGGCCUC | CCUGGGCCUC | mC*mC*mU*GGGC | chr6: 29943143- |
| | | UCCCGGGGCA | UCCCGGGGCA | CUCUCCCGGGGC | 29943163 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G019019 | 336 | UUUAGGCCA | UUUAGGCCA | mU*mU*mU*AGG | chr6: 29943188- |
| | | AAAAUCCCCC | AAAAUCCCCC | CCAAAAAUCCCC | 29943208 |
| | | C | CGUUUUAGA | CCGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021208 | 337 | CGCUGCAGCG | CGCUGCAGCG | mC*mG*mC*UGCA | chr6: 29943528- |
| | | CACGGGUACC | CACGGGUACC | GCGCACGGGUAC | 29943548 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021209 | 338 | GCUGCAGCGC | GCUGCAGCGC | mG*mC*mU*GCAG | chr6: 29943529- |
| | | ACGGGUACC | ACGGGUACC | CGCACGGGUACC | 29943549 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021210 | 339 | CUGCAGCGCA | CUGCAGCGCA | mC*mU*mG*CAGC | chr6: 29943530- |
| | | CGGGUACCA | CGGGUACCA | GCACGGGUACCA | 29943550 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018932 | 340 | CGCACGGGU | CGCACGGGU | mC*mG*mC*ACGG | chr6: 29943536- |
| | | ACCAGGGGCC | ACCAGGGGCC | GUACCAGGGGCC | 29943556 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018933 | 341 | GCACGGGUA | GCACGGGUA | mG*mC*mA*CGGG | chr6: 29943537- |
| | | CCAGGGGCCA | CCAGGGGCCA | UACCAGGGGCCA | 29943557 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | | |
| | | | AAAAAGUGG | UCAmAmCmUmU | |
| | | | CACCGAGUCG | mGmAmAmAmAm | |
| | | | GUGCUUUU | AmGmUmGmGmC | |
| | | | | mAmCmCmGmAm | |
| | | | | GmUmCmGmGmU | |
| | | | | mGmCmU*mU*mU | |
| | | | | *mU | |
| G018934 | 342 | CACGGGUACC | CACGGGUACC | mC*mA*mC*GGGU | chr6: 29943538- |
| | | AGGGGCCAC | AGGGGCCAC | ACCAGGGGCCAC | 29943558 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018935 | 343 | GGGAGGCGC | GGGAGGCGC | mG*mG*mG*AGG | chr6: 29943549- |
| | | CCCGUGGCCC | CCCGUGGCCC | CGCCCCGUGGCC | 29943569 |
| | | C | CGUUUUAGA | CCGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018936 | 344 | GCGAUCAGG | GCGAUCAGG | mG*mC*mG*AUCA | chr6: 29943556- |
| | | GAGGCGCCCC | GAGGCGCCCC | GGGAGGCGCCCC | 29943576 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021211 | 345 | UCCUUGUGG | UCCUUGUGG | mU*mC*mC*UUGU | chr6: 29943589- |
| | | GAGGCCAGCC | GAGGCCAGCC | GGGAGGCCAGCC | 29943609 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018938 | 346 | CUCCUUGUG | CUCCUUGUG | mC*mU*mC*CUUG | chr6: 29943590- |
| | | GGAGGCCAG | GGAGGCCAG | UGGGAGGCCAGC | 29943610 |
| | | CC | CCGUUUUAG | CGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018939 | 347 | GGCUGGCCUC | GGCUGGCCUC | mG*mG*mC*UGGC | chr6: 29943590- |
| | | CCACAAGGA | CCACAAGGA | CUCCCACAAGGA | 29943610 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018940 | 348 | UUGUCUCCCC | UUGUCUCCCC | mU*mU*mG*UCUC | chr6: 29943599- |
| | | UCCUUGUGG | UCCUUGUGG | CCCUCCUUGUGG | 29943619 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018941 | 349 | CCACAAGGA | CCACAAGGA | mC*mC*mA*CAAG | chr6: 29943600- |
| | | GGGGAGACA | GGGGAGACA | GAGGGGAGACAA | 29943620 |
| | | AU | AUGUUUUAG | UGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018942 | 350 | CACAAGGAG | CACAAGGAG | mC*mA*mC*AAGG | chr6: 29943601- |
| | | GGGAGACAA | GGGAGACAA | AGGGGAGACAAU | 29943621 |
| | | UU | UUGUUUUAG | UGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018943 | 351 | CAAUUGUCU | CAAUUGUCU | mC*mA*mA*UUG | chr6: 29943602- |
| | | CCCCUCCUUG | CCCCUCCUUG | UCUCCCCUCCUU | 29943622 |
| | | U | UGUUUUAGA | GUGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018944 | 352 | CCAAUUGUC | CCAAUUGUC | mC*mC*mA*AUUG | chr6: 29943603- |
| | | UCCCCUCCUU | UCCCCUCCUU | UCUCCCCUCCUU | 29943623 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018945 | 353 | AUCCCUCGAA | AUCCCUCGAA | mA*mU*mC*CCUC | chr6: 29943774- |
| | | UACUGAUGA | UACUGAUGA | GAAUACUGAUGA | 29943794 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018946 | 354 | AACCACUCAU | AACCACUCAU | mA*mA*mC*CACU | chr6: 29943779- |
| | | CAGUAUUCG | CAGUAUUCG | CAUCAGUAUUCG | 29943799 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018947 | 355 | GAACCACUCA | GAACCACUCA | mG*mA*mA*CCAC | chr6: 29943780- |
| | | UCAGUAUUC | UCAGUAUUC | UCAUCAGUAUUC | 29943800 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018948 | 356 | GAGGAAAAG | GAGGAAAAG | mG*mA*mG*GAA | chr6: 29943822- |
| | | UCACGGGCCC | UCACGGGCCC | AAGUCACGGGCC | 29943842 |
| | | A | AGUUUUAGA | CAGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018949 | 357 | GGCCCGUGAC | GGCCCGUGAC | mG*mG*mC*CCGU | chr6: 29943824- |
| | | UUUUCCUCUC | UUUUCCUCUC | GACUUUUCCUCU | 29943844 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018950 | 358 | UGCUUCACAC | UGCUUCACAC | mU*mG*mC*UUCA | chr6: 29943857- |
| | | UCAAUGUGU | UCAAUGUGU | CACUCAAUGUGU | 29943877 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018951 | 359 | GCUUCACACU | GCUUCACACU | mG*mC*mU*UCAC | chr6: 29943858- |
| | | CAAUGUGUG | CAAUGUGUG | ACUCAAUGUGUG | 29943878 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018952 | 360 | CUUCACACUC | CUUCACACUC | mC*mU*mU*CACA | chr6: 29943859- |
| | | AAUGUGUGU | AAUGUGUGU | CUCAAUGUGUGU | 29943879 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018953 | 361 | UUCACACUCA | UUCACACUCA | mU*mU*mC*ACAC | chr6: 29943860- |
| | | AUGUGUGUG | AUGUGUGUG | UCAAUGUGUGUG | 29943880 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018954 | 362 | UUGAGAAUG | UUGAGAAUG | mU*mU*mG*AGA | chr6: 29944026- |
| | | GACAGGACA | GACAGGACA | AUGGACAGGACA | 29944046 |
| | | CC | CCGUUUUAG | CCGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021205 | 363 | AGGCAUUUU | AGGCAUUUU | mA*mG*mG*CAU | chr6: 29944077- |
| | | GCAUCUGUC | GCAUCUGUC | UUUGCAUCUGUC | 29944097 |
| | | AU | AUGUUUUAG | AUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021206 | 364 | CAGGCAUUU | CAGGCAUUU | mC*mA*mG*GCAU | chr6: 29944078- |
| | | UGCAUCUGU | UGCAUCUGU | UUUGCAUCUGUC | 29944098 |
| | | CA | CAGUUUUAG | AGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018955 | 365 | AGGGGCCCU | AGGGGCCCU | mA*mG*mG*GGCC | chr6: 29944458- |
| | | GACCCUGCUA | GACCCUGCUA | CUGACCCUGCUA | 29944478 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018956 | 366 | UGGGAAAAG | UGGGAAAAG | mU*mG*mG*GAA | chr6: 29944478- |
| | | AGGGGAAGG | AGGGGAAGG | AAGAGGGGAAGG | 29944498 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018957 | 367 | UGGAGGAGG | UGGAGGAGG | mU*mG*mG*AGG | chr6: 29944597- |
| | | AAGAGCUCA | AAGAGCUCA | AGGAAGAGCUCA | 29944617 |
| | | GG | GGGUUUUAG | GGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018958 | 368 | UGAGAUUUC | UGAGAUUUC | mU*mG*mA*GAU | chr6: 29944642- |
| | | UUGUCUCAC | UUGUCUCAC | UUCUUGUCUCAC | 29944662 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018959 | 369 | GAGAUUUCU | GAGAUUUCU | mG*mA*mG*AUU | chr6: 29944643- |
| | | UGUCUCACU | UGUCUCACU | UCUUGUCUCACU | 29944663 |
| | | GA | GAGUUUUAG | GAGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018960 | 370 | UAAAGCACC | UAAAGCACC | mU*mA*mA*AGC | chr6: 29944772- |
| | | UGUUAAAAU | UGUUAAAAU | ACCUGUUAAAAU | 29944792 |
| | | GA | GAGUUUUAG | GAGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018961 | 371 | AAUCUGUCC | AAUCUGUCC | mA*mA*mU*CUG | chr6: 29944782- |
| | | UUCAUUUUA | UUCAUUUUA | UCCUUCAUUUUA | 29944802 |
| | | AC | ACGUUUUAG | ACGUUUUAGAmG | |
| | | | AGCUAGAAA | mCmUmAmGmAm | |
| | | | UAGCAAGUU | AmAm UmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018962 | 372 | GUCACAGGG | GUCACAGGG | mG*mU*mC*ACAG | chr6: 29944850- |
| | | GAAGGUCCC | GAAGGUCCC | GGGAAGGUCCCU | 29944870 |
| | | UG | UGGUUUUAG | GGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018964 | 373 | AAACAUGAA | AAACAUGAA | mA*mA*mA*CAU | chr6: 29944907- |
| | | GAAAGCAGG | GAAAGCAGG | GAAGAAAGCAGG | 29944927 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018966 | 374 | UGUCCUGUG | UGUCCUGUG | mU*mG*mU*CCUG | chr6: 29945024- |
| | | AGAUACCAG | AGAUACCAG | UGAGAUACCAGA | 29945044 |
| | | AA | AAGUUUUAG | AGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018967 | 375 | AUGAAGGAG | AUGAAGGAG | mA*mU*mG*AAG | chr6: 29945097- |
| | | GCUGAUGCC | GCUGAUGCC | GAGGCUGAUGCC | 29945117 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018968 | 376 | AGGCUGAUG | AGGCUGAUG | mA*mG*mG*CUG | chr6: 29945104- |
| | | CCUGAGGUCC | CCUGAGGUCC | AUGCCUGAGGUC | 29945124 |
| | | U | UGUUUUAGA | CUGUUUUAGAmG | |
| | | | GCUAGAAAU | mCmUmAmGmAm | |
| | | | AGCAAGUUA | AmAm UmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018969 | 377 | GGCUGAUGC | GGCUGAUGC | mG*mG*mC*UGA | chr6: 29945105- |
| | | CUGAGGUCC | CUGAGGUCC | UGCCUGAGGUCC | 29945125 |
| | | UU | UUGUUUUAG | UUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018970 | 378 | CACAAUAUCC | CACAAUAUCC | mC*mA*mC*AAUA | chr6: 29945116- |
| | | CAAGGACCUC | CAAGGACCUC | UCCCAAGGACCU | 29945136 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018971 | 379 | GGUCCUUGG | GGUCCUUGG | mG*mG*mU*CCUU | chr6: 29945118- |
| | | GAUAUUGUG | GAUAUUGUG | GGGAUAUUGUGU | 29945138 |
| | | UU | UUGUUUUAG | UGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018972 | 380 | GUCCUUGGG | GUCCUUGGG | mG*mU*mC*CUUG | chr6: 29945119- |
| | | AUAUUGUGU | AUAUUGUGU | GGAUAUUGUGUU | 29945139 |
| | | UU | UUGUUUUAG | UGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018973 | 381 | CUCCCAAACA | CUCCCAAACA | mC*mU*mC*CCAA | chr6: 29945124- |
| | | CAAUAUCCCA | CAAUAUCCCA | ACACAAUAUCCC | 29945144 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018974 | 382 | UCCUCUAGCC | UCCUCUAGCC | mU*mC*mC*UCUA | chr6: 29945176- |
| | | ACAUCUUCU | ACAUCUUCU | GCCACAUCUUCU | 29945196 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018975 | 383 | ACAGAAGAU | ACAGAAGAU | mA*mC*mA*GAA | chr6: 29945177- |
| | | GUGGCUAGA | GUGGCUAGA | GAUGUGGCUAGA | 29945197 |
| | | GG | GGGUUUUAG | GGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018976 | 384 | CCUCUAGCCA | CCUCUAGCCA | mC*mC*mU*CUAG | chr6: 29945177- |
| | | CAUCUUCUG | CAUCUUCUG | CCACAUCUUCUG | 29945197 |
| | | U | UGUUUUAGA | UGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018977 | 385 | CCCACAGAAG | CCCACAGAAG | mC*mC*mC*ACAG | chr6: 29945180- |
| | | AUGUGGCUA | AUGUGGCUA | AAGAUGUGGCUA | 29945200 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018978 | 386 | GUCAGAUCCC | GUCAGAUCCC | mG*mU*mC*AGA | chr6: 29945187- |
| | | ACAGAAGAU | ACAGAAGAU | UCCCACAGAAGA | 29945207 |
| | | G | GGUUUUAGA | UGGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018979 | 387 | AUCUUCUGU | AUCUUCUGU | mA*mU*mC*UUCU | chr6: 29945188- |
| | | GGGAUCUGA | GGGAUCUGA | GUGGGAUCUGAC | 29945208 |
| | | CC | CCGUUUUAG | CGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018980 | 388 | CCCAGGCAGU | CCCAGGCAGU | mC*mC*mC*AGGC | chr6: 29945228- |
| | | GACAGUGCCC | GACAGUGCCC | AGUGACAGUGCC | 29945248 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018981 | 389 | CUGGGCACU | CUGGGCACU | mC*mU*mG*GGCA | chr6: 29945230- |
| | | GUCACUGCCU | GUCACUGCCU | CUGUCACUGCCU | 29945250 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018982 | 390 | CCUGGGCACU | CCUGGGCACU | mC*mC*mU*GGGC | chr6: 29945231- |
| | | GUCACUGCCU | GUCACUGCCU | ACUGUCACUGCC | 29945251 |
| | | | GUUUUAGAG | UGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021207 | 391 | CCCUGGGCAC | CCCUGGGCAC | mC*mC*mC*UGGG | chr6: 29945232- |
| | | UGUCACUGCC | UGUCACUGCC | CACUGUCACUGC | 29945252 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G018987 | 392 | UUGGGUGUU | UUGGGUGUU | mU*mU*mG*GGU | chr6: 29945308- |
| | | GGGCGGAAC | GGGCGGAAC | GUUGGGCGGAAC | 29945328 |
| | | AG | AGGUUUUAG | AGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018988 | 393 | UGGAUGUAU | UGGAUGUAU | mU*mG*mG*AUG | chr6: 29945361- |
| | | UGAGCAUGC | UGAGCAUGC | UAUUGAGCAUGC | 29945381 |
| | | GA | GAGUUUUAG | GAGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018989 | 394 | GGAUGUAUU | GGAUGUAUU | mG*mG*mA*UGU | chr6: 29945362- |
| | | GAGCAUGCG | GAGCAUGCG | AUUGAGCAUGCG | 29945382 |
| | | AU | AUGUUUUAG | AUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G018963 | 395 | AACAUGAAG | AACAUGAAG | mA*mA*mC*AUG | chr6: 31382543- |
| | | AAAGCAGGU | AAAGCAGGU | AAGAAAGCAGGU | 31382563 |
| | | GU | GUGUUUUAG | GUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021885 | 396 | UAGCCCACGG | UAGCCCACGG | mU*mA*mG*CCCA | chr6: 29942815- |
| | | CGAUGAAGC | CGAUGAAGC | CGGCGAUGAAGC | 29942835 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021886 | 397 | GUAGCCCACG | GUAGCCCACG | mG*mU*mA*GCCC | chr6: 29942816- |
| | | GCGAUGAAG | GCGAUGAAG | ACGGCGAUGAAG | 29942836 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021887 | 398 | CGUAGCCCAC | CGUAGCCCAC | mC*mG*mU*AGCC | chr6: 29942817- |
| | | GGCGAUGAA | GGCGAUGAA | CACGGCGAUGAA | 29942837 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021888 | 399 | CUUCAUCGCC | CUUCAUCGCC | mC*mU*mU*CAUC | chr6: 29942817- |
| | | GUGGGCUAC | GUGGGCUAC | GCCGUGGGCUAC | 29942837 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021889 | 400 | CGUGUCGUCC | CGUGUCGUCC | mC*mG*mU*GUCG | chr6: 29942828- |
| | | ACGUAGCCCA | ACGUAGCCCA | UCCACGUAGCCC | 29942848 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021890 | 401 | UGGACGACA | UGGACGACA | mU*mG*mG*ACG | chr6: 29942837- |
| | | CGCAGUUCG | CGCAGUUCG | ACACGCAGUUCG | 29942857 |
| | | UG | UGGUUUUAG | UGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021891 | 402 | GGAUGGAGC | GGAUGGAGC | mG*mG*mA*UGG | chr6: 29942885- |
| | | CGCGGGCGCC | CGCGGGCGCC | AGCCGCGGGCGC | 29942905 |
| | | G | GGUUUUAGA | CGGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021892 | 403 | GCGGGCGCCG | GCGGGCGCCG | mG*mC*mG*GGCG | chr6: 29942895- |
| | | UGGAUAGAG | UGGAUAGAG | CCGUGGAUAGAG | 29942915 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021893 | 404 | UGCUCUAUCC | UGCUCUAUCC | mU*mG*mC*UCUA | chr6: 29942896- |
| | | ACGGCGCCCG | ACGGCGCCCG | UCCACGGCGCCC | 29942916 |
| | | | GUUUUAGAG | GGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021894 | 405 | GGCGCCGUG | GGCGCCGUG | mG*mG*mC*GCCG | chr6: 29942898- |
| | | GAUAGAGCA | GAUAGAGCA | UGGAUAGAGCAG | 29942918 |
| | | GG | GGGUUUUAG | GGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021895 | 406 | GCGCCGUGG | GCGCCGUGG | mG*mC*mG*CCGU | chr6: 29942899- |
| | | AUAGAGCAG | AUAGAGCAG | GGAUAGAGCAGG | 29942919 |
| | | GA | GAGUUUUAG | AGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021896 | 407 | CGCCGUGGA | CGCCGUGGA | mC*mG*mC*CGUG | chr6: 29942900- |
| | | UAGAGCAGG | UAGAGCAGG | GAUAGAGCAGGA | 29942920 |
| | | AG | AGGUUUUAG | GGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021897 | 408 | GUGGAUAGA | GUGGAUAGA | mG*mU*mG*GAU | chr6: 29942904- |
| | | GCAGGAGGG | GCAGGAGGG | AGAGCAGGAGGG | 29942924 |
| | | GC | GCGUUUUAG | GCGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021898 | 409 | GGCCCCUCCU | GGCCCCUCCU | mG*mG*mC*CCCU | chr6: 29942905- |
| | | GCUCUAUCCA | GCUCUAUCCA | CCUGCUCUAUCC | 29942925 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021899 | 410 | AGCAGGAGG | AGCAGGAGG | mA*mG*mC*AGG | chr6: 29942912- |
| | | GGCCGGAGU | GGCCGGAGU | AGGGGCCGGAGU | 29942932 |
| | | AU | AUGUUUUAG | AUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021900 | 411 | GCAGGAGGG | GCAGGAGGG | mG*mC*mA*GGA | chr6: 29942913- |
| | | GCCGGAGUA | GCCGGAGUA | GGGGCCGGAGUA | 29942933 |
| | | UU | UUGUUUUAG | UUGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021901 | 412 | GGAGUGGCU | GGAGUGGCU | mG*mG*mA*GUG | chr6: 29943490- |
| | | CCGCAGAUAC | CCGCAGAUAC | GCUCCGCAGAUA | 29943510 |
| | | C | CGUUUUAGA | CCGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021902 | 413 | CUCCGCAGAU | CUCCGCAGAU | mC*mU*mC*CGCA | chr6: 29943497- |
| | | ACCUGGAGA | ACCUGGAGA | GAUACCUGGAGA | 29943517 |
| | | A | AGUUUUAGA | AGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021903 | 414 | UCCGCAGAU | UCCGCAGAU | mU*mC*mC*GCAG | chr6: 29943498- |
| | | ACCUGGAGA | ACCUGGAGA | AUACCUGGAGAA | 29943518 |
| | | AC | ACGUUUUAG | CGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021904 | 415 | CAGAUACCU | CAGAUACCU | mC*mA*mG*AUAC | chr6: 29943502- |
| | | GGAGAACGG | GGAGAACGG | CUGGAGAACGGG | 29943522 |
| | | GA | GAGUUUUAG | AGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021905 | 416 | UCCCGUUCUC | UCCCGUUCUC | mU*mC*mC*CGUU | chr6: 29943502- |
| | | CAGGUAUCU | CAGGUAUCU | CUCCAGGUAUCU | 29943522 |
| | | G | GGUUUUAGA | GGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021906 | 417 | GCGUCUCCUU | GCGUCUCCUU | mG*mC*mG*UCUC | chr6: 29943511- |
| | | CCCGUUCUCC | CCCGUUCUCC | CUUCCCGUUCUC | 29943531 |
| | | | GUUUUAGAG | CGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021907 | 418 | GAAGGAGAC | GAAGGAGAC | mG*mA*mA*GGA | chr6: 29943520- |
| | | GCUGCAGCGC | GCUGCAGCGC | GACGCUGCAGCG | 29943540 |
| | | A | AGUUUUAGA | CAGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021908 | 419 | AAGGAGACG | AAGGAGACG | mA*mA*mG*GAG | chr6: 29943521- |
| | | CUGCAGCGCA | CUGCAGCGCA | ACGCUGCAGCGC | 29943541 |
| | | C | CGUUUUAGA | ACGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021909 | 420 | AGAUCUACA | AGAUCUACA | mA*mG*mA*UCU | chr6: 29943566- |
| | | GGCGAUCAG | GGCGAUCAG | ACAGGCGAUCAG | 29943586 |
| | | GG | GGGUUUUAG | GGGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021910 | 421 | UGAUCGCCU | UGAUCGCCU | mU*mG*mA*UCGC | chr6: 29943569- |
| | | GUAGAUCUC | GUAGAUCUC | CUGUAGAUCUCC | 29943589 |
| | | CC | CCGUUUUAG | CGUUUUAGAmGm | |
| | | | AGCUAGAAA | CmUmAmGmAmA | |
| | | | UAGCAAGUU | mAmUmAmGmCA | |
| | | | AAAAUAAGG | AGUUAAAAUAAG | |
| | | | CUAGUCCGU | GCUAGUCCGUUA | |
| | | | UAUCAACUU | UCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021911 | 422 | GGGAGAUCU | GGGAGAUCU | mG*mG*mG*AGA | chr6: 29943569- |
| | | ACAGGCGAU | ACAGGCGAU | UCUACAGGCGAU | 29943589 |
| | | CA | CAGUUUUAG | CAGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021912 | 423 | CGGGAGAUC | CGGGAGAUC | mC*mG*mG*GAG | chr6: 29943570- |
| | | UACAGGCGA | UACAGGCGA | AUCUACAGGCGA | 29943590 |
| | | UC | UCGUUUUAG | UCGUUUUAGAmG | |
| | | | AGCUAGAAA | mCmUmAmGmAm | |
| | | | UAGCAAGUU | AmAmUmAmGmC | |
| | | | AAAAUAAGG | AAGUUAAAAUAA | |
| | | | CUAGUCCGU | GGCUAGUCCGUU | |
| | | | UAUCAACUU | AUCAmAmCmUmU | |
| | | | GAAAAAGUG | mGmAmAmAmAm | |
| | | | GCACCGAGUC | AmGmUmGmGmC | |
| | | | GGUGCUUUU | mAmCmCmGmAm | |
| | | | | GmUmCmGmGmU | |
| | | | | mGmCmU*mU*mU | |
| | | | | *mU | |
| G021913 | 424 | CGCCUGUAG | CGCCUGUAG | mC*mG*mC*CUGU | chr6: 29943573- |
| | | AUCUCCCGGG | AUCUCCCGGG | AGAUCUCCCGGG | 29943593 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021914 | 425 | GGCCAGCCCG | GGCCAGCCCG | mG*mG*mC*CAGC | chr6: 29943578- |
| | | GGAGAUCUA | GGAGAUCUA | CCGGGAGAUCUA | 29943598 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021915 | 426 | UCCCGGGCUG | UCCCGGGCUG | mU*mC*mC*CGGG | chr6: 29943585- |
| | | GCCUCCCACA | GCCUCCCACA | CUGGCCUCCCAC | 29943605 |
| | | | GUUUUAGAG | AGUUUUAGAmGm | |
| | | | CUAGAAAUA | CmUmAmGmAmA | |
| | | | GCAAGUUAA | mAmUmAmGmCA | |
| | | | AAUAAGGCU | AGUUAAAAUAAG | |
| | | | AGUCCGUUA | GCUAGUCCGUUA | |
| | | | UCAACUUGA | UCAmAmCmUmU | |
| | | | AAAAGUGGC | mGmAmAmAmAm | |
| | | | ACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021916 | 427 | GGGCUGGCC | GGGCUGGCC | mG*mG*mG*CUG | chr6: 29943589- |
| | | UCCCACAAGG | UCCCACAAGG | GCCUCCCACAAG | 29943609 |
| | | A | AGUUUUAGA | GAGUUUUAGAmG | |
| | | | GCUAGAAAU | mCmUmAmGmAm | |
| | | | AGCAAGUUA | AmAmUmAmGmC | |
| | | | AAAUAAGGC | AAGUUAAAAUAA | |
| | | | UAGUCCGUU | GGCUAGUCCGUU | |
| | | | AUCAACUUG | AUCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |
| G021917 | 428 | CUGAUCGCCU | CUGAUCGCCU | mC*mU*mG*AUCG | chr6: 29943568- |
| | | GUAGAUCUC | GUAGAUCUC | CCUGUAGAUCUC | 29943588 |
| | | C | CGUUUUAGA | CGUUUUAGAmGm | |
| | | | GCUAGAAAU | CmUmAmGmAmA | |
| | | | AGCAAGUUA | mAmUmAmGmCA | |
| | | | AAAUAAGGC | AGUUAAAAUAAG | |
| | | | UAGUCCGUU | GCUAGUCCGUUA | |
| | | | AUCAACUUG | UCAmAmCmUmU | |
| | | | AAAAAGUGG | mGmAmAmAmAm | |
| | | | CACCGAGUCG | AmGmUmGmGmC | |
| | | | GUGCUUUU | mAmCmCmGmAm | |
| | | | | GmUmCmGmGmU | |
| | | | | mGmCmU*mU*mU | |
| | | | | *mU | |

*The guide sequence disclosed in this Table may be unmodified, modified with the exemplary modification
pattern shown in the Table, or modified with a different modification pattern disclosed herein or
available in the art.

TABLE 5A
Additional exemplary HLA-A guide sequences

| SEQ ID | | | RNA-guided DNA | Genomic |
|---|---|---|---|---|
| NO | Guide Sequence | PAM | binding agent | Coordinates (hg38) |
| 429 | AGGAUGGAGCCGCGGGCGCC | GTGGA | S. aureus Cas9 | chr6: 29942884- |
| | | T | | 29942904 |
| 430 | GGAAGGAGACGCUGCAGCGC | ACGGG | S. aureus Cas9 | chr6: 29943519- |
| | | T | | 29943539 |
| 431 | GACAGCGACGCCGCGAGCCA | GAGGA | S. aureus Cas9 | chr6: 29942863- |
| | | T | | 29942883 |
| 432 | CGGGAAGGAGACGCUGCAGC | TTCT | CasX | chr6: 29943517- |
| | | | | 29943537 |
| 433 | CCGUGCGCUGCAGCGUCUCC | TTCC | CasX | chr6: 29943523- |
| | | | | 29943543 |
| 434 | ACGCAGUUCGUGCGGUUCGA | NNNNC | NME2 | chr6: 29942845- |
| | CAGC | C | | 29942869 |
| 435 | UCGUGCGGUUCGACAGCGAC | NNNNC | NME2 | chr6: 29942852- |
| | GCCG | C | | 29942876 |
| 436 | CAGCGACGCCGCGAGCCAGA | NNNNC | NME2 | chr6: 29942865- |
| | GGAU | C | | 29942889 |
| 437 | GCUCUAUCCACGGCGCCCGC | NNNNC | NME2 | chr6: 29942891- |
| | GGCU | C | | 29942915 |
| 438 | UCCUGCUCUAUCCACGGCGC | NNNNC | NME2 | chr6: 29942895- |
| | CCGC | C | | 29942919 |
| 439 | CCGGCCCCUCCUGCUCUAUC | NNNNC | NME2 | chr6: 29942903- |
| | CACG | C | | 29942927 |
| 440 | UCCGGCCCCUCCUGCUCUAU | NNNNC | NME2 | chr6: 29942904- |
| | CCAC | C | | 29942928 |
| 441 | GGGAAGGAGACGCUGCAGCG | NNNNC | NME2 | chr6: 29943518- |
| | CACG | C | | 29943542 |
| 442 | AGACGCUGCAGCGCACGGGU | NNNNC | NME2 | chr6: 29943525- |
| | ACCA | C | | 29943549 |
| 443 | GCGCACGGGUACCAGGGGCC | NNNNC | NME2 | chr6: 29943535- |
| | ACGG | C | | 29943559 |
| 444 | CACGGGUACCAGGGGCCACG | NNNNC | NME2 | chr6: 29943538- |
| | GGGC | C | | 29943562 |
| 445 | ACGGGUACCAGGGGCCACGG | NNNNC | NME2 | chr6: 29943539- |
| | GGCG | C | | 29943563 |
| 446 | CAGGGGCCACGGGGCGCCUC | NNNNC | NME2 | chr6: 29943547- |
| | CCUG | C | | 29943571 |
| 447 | CAGGGAGGCGCCCCGUGGCC | NNNNC | NME2 | chr6: 29943547- |
| | CCUG | C | | 29943571 |
| 448 | UCAGGGAGGCGCCCCGUGGC | NNNNC | NME2 | chr6: 29943548- |
| | CCCU | C | | 29943572 |
| 449 | CAGGCGAUCAGGGAGGCGCC | NNNNC | NME2 | chr6: 29943555- |
| | CCGU | C | | 29943579 |
| 450 | ACAGGCGAUCAGGGAGGCGC | NNNNC | NME2 | chr6: 29943556- |
| | CCCG | C | | 29943580 |
| 451 | UACAGGCGAUCAGGGAGGCG | NNNNC | NME2 | chr6: 29943557- |
| | CCCC | C | | 29943581 |
| 452 | GGGCGCCUCCCUGAUCGCCU | NNNNC | NME2 | chr6: 29943558- |
| | GUAG | C | | 29943582 |
| 453 | GGCGCCUCCCUGAUCGCCUG | NNNNC | NME2 | chr6: 29943559- |
| | UAGA | C | | 29943583 |
| 454 | GAGAUCUACAGGCGAUCAGG | NNNNC | NME2 | chr6: 29943563- |
| | GAGG | C | | 29943587 |
| 455 | GGAGAUCUACAGGCGAUCAG | NNNNC | NME2 | chr6: 29943564- |
| | GGAG | C | | 29943588 |
| 456 | GGGAGAUCUACAGGCGAUCA | NNNNC | NME2 | chr6: 29943565- |
| | GGGA | C | | 29943589 |
| 457 | CUGAUCGCCUGUAGAUCUCC | NNNNC | NME2 | chr6: 29943568- |
| | CGGG | C | | 29943592 |
| 458 | AUCGCCUGUAGAUCUCCCGG | NNNNC | NME2 | chr6: 29943571- |
| | GCUG | C | | 29943595 |
| 459 | UCGCCUGUAGAUCUCCCGGG | NNNNC | NME2 | chr6: 29943572- |
| | CUGG | C | | 29943596 |
| 460 | UUGUCUCCCCUCCUUGUGGG | NNNNC | NME2 | chr6: 29943595- |
| | AGGC | C | | 29943619 |
| 461 | AUUGUCUCCCCUCCUUGUGG | NNNNC | NME2 | chr6: 29943596- |
| | GAGG | C | | 29943620 |
| 462 | CCCAAUUGUCUCCCCUCCUU | NNNNC | NME2 | chr6: 29943600- |
| | GUGG | C | | 29943624 |

TABLE 5B
Exemplary Spy HLA-A guide sequences

| | | | | |
|---|---|---|---|---|
| 463 | GGAUGGAGCCGCGGGCGCCG | NGG | Spy + | chr6: |
| | | | Base_ | 29942885- |
| | | | Editor | 29942905 |
| 464 | GCGGGCGCCGUGGAUAGAGC | NGG | Spy + | chr6: |
| | | | Base_ | 29942895- |
| | | | Editor | 29942915 |
| 465 | UGCUCUAUCCACGGCGCCCG | NGG | Spy + | chr6: |
| | | | Base_ | 29942896- |
| | | | Editor | 29942916 |
| 466 | GGCGCCGUGGAUAGAGCAGG | NGG | Spy + | chr6: |
| | | | Base_ | 29942898- |
| | | | Editor | 29942918 |
| 467 | GCGCCGUGGAUAGAGCAGGA | NGG | Spy + | chr6: |
| | | | Base_ | 29942899- |
| | | | Editor | 29942919 |
| 468 | CGCCGUGGAUAGAGCAGGAG | NGG | Spy + | chr6: |
| | | | Base_ | 29942900- |
| | | | Editor | 29942920 |
| 469 | GUGGAUAGAGCAGGAGGGGC | NGG | Spy + | chr6: |
| | | | Base_ | 29942904- |
| | | | Editor | 29942924 |
| 470 | GCGUCUCCUUCCCGUUCUCC | NGG | Spy + | chr6: |
| | | | Base_ | 29943511- |
| | | | Editor | 29943531 |
| 471 | GAAGGAGACGCUGCAGCGCA | NGG | Spy + | chr6: |
| | | | Base_ | 29943520- |
| | | | Editor | 29943540 |
| 472 | AAGGAGACGCUGCAGCGCAC | NGG | Spy + | chr6: |
| | | | Base_ | 29943521- |
| | | | Editor | 29943541 |
| 473 | GCUGCAGCGCACGGGUACCA | NGG | Spy + | chr6: |
| | | | Base_ | 29943529- |
| | | | Editor | 29943549 |
| 474 | AGAUCUACAGGCGAUCAGGG | NGG | Spy + | chr6: |
| | | | Base_ | 29943566- |
| | | | Editor | 29943586 |
| 475 | CUGAUCGCCUGUAGAUCUCC | NGG | Spy + | chr6: |
| | | | Base_ | 29943568- |
| | | | Editor | 29943588 |
| 476 | UGAUCGCCUGUAGAUCUCCC | NGG | Spy + | chr6: |
| | | | Base_ | 29943569- |
| | | | Editor | 29943589 |
| 477 | GGGAGAUCUACAGGCGAUCA | NGG | Spy + | chr6: |
| | | | Base_ | 29943569- |
| | | | Editor | 29943589 |
| 478 | CGGGAGAUCUACAGGCGAUC | NGG | Spy + | chr6: |
| | | | Base_ | 29943570- |
| | | | Editor | 29943590 |
| 479 | CGCCUGUAGAUCUCCCGGGC | NGG | Spy + | chr6: |
| | | | Base_ | 29943573- |
| | | | Editor | 29943593 |
| 480 | GGCCAGCCCGGGAGAUCUAC | NGG | Spy + | chr6: |
| | | | Base_ | 29943578- |
| | | | Editor | 29943598 |
| 481 | UCCCGGGCUGGCCUCCCACA | NGG | Spy + | chr6: |
| | | | Base_ | 29943585- |
| | | | Editor | 29943605 |
| 482 | GGGCUGGCCUCCCACAAGGA | NGG | Spy + | chr6: |
| | | | Base_ | 29943589- |
| | | | Editor | 29943609 |

[000632] Table 6. Additional Exemplary HLA-A guide sequences.

| | | | | Exemplary Guide RNA | |
|---|---|---|---|---|---|
| | | | Exemplary | Modified Sequence | |
| | | | Guide RNA Full | (four terminal U | |
| | | | Sequence | residues are optional | |
| | SEQ ID NO | | with PAM | and may include 0, | |
| | to the | | (SEQ ID NOs: | 1, 2, 3, 4, or more Us) | Genomic |
| Guide | Guide | Guide | 1483-1498 and | (SEQ ID NOs: 2483-2498 | Coordinates |
| ID | Sequence | Sequence | 1500-1511) | and 2500-2511) | (hg38) |
| G021857 | 483 | ACGACAC | ACGACACUGAUU | mA*mC*mG*ACACUGAU | chr6: 29942469- |
| | | UGAUUGG | GGCUUCUCGUUU | UGGCUUCUCGUUUUAG | 29942489 |
| | | CUUCUC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021858 | 484 | ACCCCUC | ACCCCUCAUCCC | mA*mC*mC*CCUCAUCC | chr6: 29943058- |
| | | AUCCCCC | CCACGGACGUUU | CCCACGGACGUUUUAG | 29943078 |
| | | ACGGAC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021859 | 485 | GGCCCGU | GGCCCGUCCGUG | mG*mG*mC*CCGUCCGU | chr6: 29943063- |
| | | CCGUGGG | GGGGAUGAGUUU | GGGGGAUGAGUUUUAG | 29943083 |
| | | GGAUGA | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021860 | 486 | GCCAGGU | GCCAGGUCGCCC | mG*mC*mC*AGGUCGCC | chr6: 29943080- |
| | | CGCCCAC | ACAGUCUCGUUU | CACAGUCUCGUUUUAG | 29943100 |
| | | AGUCUC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021861 | 487 | GUUUAGG | GUUUAGGCCAAA | mG*mU*mU*UAGGCCAA | chr6: 29943187- |
| | | CCAAAAA | AAUCCCCCGUUU | AAAUCCCCCGUUUUAG | 29943207 |
| | | UCCCCC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021862 | 488 | GGCCAAA | GGCCAAAAAUCC | mG*mG*mC*CAAAAAUC | chr6: 29943192- |
| | | AAUCCCC | CCCCGGGUGUUU | CCCCCGGGUGUUUUAG | 29943212 |
| | | CCGGGU | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021863 | 489 | GACCAAC | GACCAACCCGGG | mG*mA*mC*CAACCCGG | chr6: 29943197- |
| | | CCGGGGG | GGGAUUUUGUUU | GGGGAUUUUGUUUUAG | 29943217 |
| | | GAUUUU | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021864 | 490 | CACGGGC | CACGGGCCCAAG | mC*mA*mC*GGGCCCAA | chr6: 29943812- |
| | | CCAAGGC | GCUGCUGCGUUU | GGCUGCUGCGUUUUAG | 29943832 |
| | | UGCUGC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021865 | 491 | ACCCUCA | ACCCUCAUGCUG | mA*mC*mC*CUCAUGCU | chr6: 29944349- |
| | | UGCUGCA | CACAUGGCGUUU | GCACAUGGCGUUUUAG | 29944369 |
| | | CAUGGC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021866 | 492 | CCUCUAG | CCUCUAGGACCU | mC*mC*mU*CUAGGACC | chr6: 29944996- |
| | | GACCUUA | UAAGGCCCGUUU | UUAAGGCCCGUUUUAG | 29945016 |
| | | AGGCCC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021867 | 493 | GCUCCUU | GCUCCUUUCUGG | mG*mC*mU*CCUUUCUG | chr6: 29945018- |
| | | UCUGGUA | UAUCUCACGUUU | GUAUCUCACGUUUUAG | 29945038 |
| | | UCUCAC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021868 | 494 | GCUAUGG | GCUAUGGGGUUU | mG*mC*mU*AUGGGGUU | chr6: 29945341- |
| | | GGUUUCU | CUUUGCAUGUUU | UCUUUGCAUGUUUUAG | 29945361 |
| | | UUGCAU | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021869 | 495 | GCCUUUG | GCCUUUGCAGAA | mG*mC*mC*UUUGCAGA | chr6: 29945526- |
| | | CAGAAAC | ACAAAGUCGUUU | AACAAAGUCGUUUUAG | 29945546 |
| | | AAAGUC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021870 | 496 | UGGACCA | UGGACCAACCGC | mU*mG*mG*ACCAACCG | chr6: 29944880- |
| | | ACCGCCC | CCUCCUGAGUUU | CCCUCCUGAGUUUUAG | 29944900 |
| | | UCCUGA | UAGAGCUAGAAA | AmGmCmUmAmGmAmAm | (mismatch to |
| | | | UAGCAAGUUAAA | AmUmAmGmCAAGUUAA | hg38 = 2) |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021871 | 497 | AGCCUCU | AGCCUCUCUGAC | mA*mG*mC*CUCUCUGA | Na |
| | | CUGACCU | CUUUAGCAGUUU | CCUUUAGCAGUUUUAG | |
| | | UUAGCA | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021872 | 498 | CGCCCUC | CGCCCUCCUGAA | mC*mG*mC*CCUCCUGA | Na |
| | | CUGAAGG | GGUCCUCAGUUU | AGGUCCUCAGUUUUAG | |
| | | UCCUCA | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021873 | 500 | CCGCCCU | CCGCCCUCCUGA | mC*mC*mG*CCCUCCUG | Na |
| | | CCUGAAG | AGGUCCUCGUUU | AAGGUCCUCGUUUUAG | |
| | | GUCCUC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021874 | 501 | UGGUUCC | UGGUUCCCUUUG | mU*mG*mG*UUCCCUUU | chr6: 29943794- |
| | | CUUUGAC | ACACACACGUUU | GACACACACGUUUUAG | 29943814 |
| | | ACACAC | UAGAGCUAGAAA | AmGmCmUmAmGmAmAm | (mismatch to |
| | | | UAGCAAGUUAAA | AmUmAmGmCAAGUUAA | hg38 = 3) |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021875 | 502 | GACCCUG | GACCCUGCUAAA | mG*mA*mC*CCUGCUAA | na |
| | | CUAAAGG | GGUCAGAGGUUU | AGGUCAGAGGUUUUAG | |
| | | UCAGAG | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021876 | 503 | AGGACCU | AGGACCUUCAGG | mA*mG*mG*ACCUUCAG | na |
| | | UCAGGAG | AGGGCGGUGUUU | GAGGGCGGUGUUUUAG | |
| | | GGCGGU | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021877 | 504 | GCACACU | GCACACUUCUAC | mG*mC*mA*CACUUCUA | chr6: 29944671- |
| | | UCUACCU | CUGGGUCUGUUU | CCUGGGUCUGUUUUAG | 29944691 |
| | | GGGUCU | UAGAGCUAGAAA | AmGmCmUmAmGmAmAm | (mismatch to |
| | | | UAGCAAGUUAAA | AmUmAmGmCAAGUUAA | hg38 = 3) |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021878 | 505 | GAGCCUC | GAGCCUCUCUGA | mG*mA*mG*CCUCUCUG | na |
| | | UCUGACC | CCUUUAGCGUUU | ACCUUUAGCGUUUUAG | |
| | | UUUAGC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021879 | 506 | ACACUCC | ACACUCCUCCAG | mA*mC*mA*CUCCUCCA | chr6: 29944054- |
| | | UCCAGCA | CACACAUGGUUU | GCACACAUGGUUUUAG | 29944074 |
| | | CACAUG | UAGAGCUAGAAA | AmGmCmUmAmGmAmAm | (mismatch to |
| | | | UAGCAAGUUAAA | AmUmAmGmCAAGUUAA | hg38 = 2) |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAm AmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021880 | 507 | CUCUGAC | CUCUGACCUUUA | mC*mU*mC*UGACCUUU | na |
| | | CUUUAGC | GCAGGGUCGUUU | AGCAGGGUCGUUUUAG | |
| | | AGGGUC | UAGAGCUAGAAA | AmGmCmUmAmGmAmAm | |
| | | | UAGCAAGUUAAA | AmUmAmGmCAAGUUAA | |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021881 | 508 | CAAGAUA | CAAGAUAGCCAC | mC*mA*mA*GAUAGCCA | chr6: 29944043- |
| | | GCCACAU | AUGUGUGCGUUU | CAUGUGUGCGUUUUAG | 29944063 |
| | | GUGUGC | UAGAGCUAGAAA | AmGmCmUmAmGmAmAm | (mismatch to |
| | | | UAGCAAGUUAAA | AmUmAmGmCAAGUUAA | hg38 = 2) |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021882 | 509 | UCUGACC | UCUGACCUUUAG | mU*mC*mU*GACCUUUA | chr6: 29944450- |
| | | UUUAGCA | CAGGGUCAGUUU | GCAGGGUCAGUUUUAG | 29944470 |
| | | GGGUCA | UAGAGCUAGAAA | AmGmCmUmAmGmAmAm | (mismatch to |
| | | | UAGCAAGUUAAA | AmUmAmGmCAAGUUAA | hg38 = 3) |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021883 | 510 | UGUAAAG | UGUAAAGGUGAG | mU*mG*mU*AAAGGUGA | chr6: 29945274- |
| | | GUGAGAG | AGCCUGGAGUUU | GAGCCUGGAGUUUUAG | 29945294 |
| | | CCUGGA | UAGAGCUAGAAA | AmGmCmUmAmGmAmAm | (mismatch to |
| | | | UAGCAAGUUAAA | AmUmAmGmCAAGUUAA | hg38 = 1) |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |
| G021884 | 511 | GAAGGUC | GAAGGUCCCUGA | mG*mA*mA*GGUCCCUG | chr6: 29944859- |
| | | CCUGAGG | GGACCUUCGUUU | AGGACCUUCGUUUUAG | 29944879 |
| | | ACCUUC | UAGAGCUAGAAA | AmGmCmUmAmGmAmAm | (mismatch to |
| | | | UAGCAAGUUAAA | AmUmAmGmCAAGUUAA | hg38 = 3) |
| | | | AUAAGGCUAGUC | AAUAAGGCUAGUCCGU | |
| | | | CGUUAUCAACUU | UAUCAmAmCmUmUmGm | |
| | | | GAAAAAGUGGCA | AmAmAmAmAmGmUmG | |
| | | | CCGAGUCGGUGC | mGmCmAmCmCmGmAmG | |
| | | | UUUU | mUmCmGmGmUmGmCmU | |
| | | | | *mU*mU*mU | |

*The guide sequence disclosed in this Table may be unmodified, modified with the exemplary modification pattern shown in the Table, or modified with a different modification pattern disclosed herein or available in the art.

TABLE 7
Additional Exemplary HLA-A Nme guide sequences

| | SEQ ID | | Exemplary | Exemplary Guide | |
|---|---|---|---|---|---|
| | NO to | | Guide RNA Full | RNA Modified | |
| | the | | Sequence | Sequence | Genomic |
| Guide | Guide | Guide | (SEQ ID NOs: | (SEQ ID NOs: | Coordinates |
| ID | Sequence | Sequence | 1512-1590) | 2512-2590) | (hg38) |
| G028854 | 512 | CCUGGGUCU | CCUGGGUCU | mC*mC*mU*mGmGG | chr6:29944223- |
| | | GGUCCUCCCC | GGUCCUCCCC | UmCmUGmGUmCCU | 29944247 |
| | | AUCCC | AUCCCGUUG | CCmCCAUmCCCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028855 | 513 | GUGGAGACC | GUGGAGACC | mG*mU*mG*mGmA | chr6:29944264- |
| | | AGGCCUGCA | AGGCCUGCA | GAmCmCAmGGmCC | 29944288 |
| | | GGGGAU | GGGGAUGUU | UGCmAGGGmGAUm | |
| | | | GUAGCUCCCU | GUUGmUmAmGmCU | |
| | | | GAAACCGUU | CCCmUmGmAmAmA | |
| | | | GCUACAAUA | mCmCGUUmGmCUA | |
| | | | AGGCCGUCG | mCAAU*AAGmGmC | |
| | | | AAAGAUGUG | CmGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028856 | 514 | CCGUGUCCUG | CCGUGUCCUG | mC*mC*mG*mUmGU | chr6:29944229- |
| | | GGUCUGGUC | GGUCUGGUC | CmCmUGmGGmUCU | 29944253 |
| | | CUCCC | CUCCCGUUGU | GGmUCCUmCCCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028857 | 515 | UCCGUGUCCU | UCCGUGUCCU | mU*mC*mC*mGmUG | chr6:29944230- |
| | | GGGUCUGGU | GGGUCUGGU | UmCmCUmGGmGUC | 29944254 |
| | | CCUCC | CCUCCGUUGU | UGmGUCCmUCCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028858 | 516 | CACAUGGCA | CACAUGGCA | mC*mA*mC*mAmUG | chr6:29944327- |
| | | GGUGUAUCU | GGUGUAUCU | GmCmAGmGUmGUA | 29944351 |
| | | CUGCUC | CUGCUCGUU | UCmUCUGmCUCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028859 | 517 | UGCUGCACA | UGCUGCACA | mU*mG*mC*mUmGC | chr6:29944332- |
| | | UGGCAGGUG | UGGCAGGUG | AmCmAUmGGmCAG | 29944356 |
| | | UAUCUC | UAUCUCGUU | GUmGUAUmCUCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028860 | 518 | CCGCACGAAC | CCGCACGAAC | mC*mC*mG*mCmAC | chr6:29942830- |
| | | UGCGUGUCG | UGCGUGUCG | GmAmACmUGmCGU | 29942854 |
| | | UCCAC | UCCACGUUG | GUmCGUCmCACmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028861 | 519 | CGCACGAACU | CGCACGAACU | mC*mG*mC*mAmCG | chr6:29942829- |
| | | GCGUGUCGU | GCGUGUCGU | AmAmCUmGCmGUG | 29942853 |
| | | CCACG | CCACGGUUG | UCmGUCCmACGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028862 | 520 | GUGCGCUGC | GUGCGCUGC | mG*mU*mG*mCmGC | chr6:29943511- |
| | | AGCGUCUCCU | AGCGUCUCCU | UmGmCAmGCmGUC | 29943535 |
| | | UCCCG | UCCCGGUUG | UCmCUUCmCCGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028863 | 521 | CCGAGGAUG | CCGAGGAUG | mC*mC*mG*mAmGG | chr6:29942547- |
| | | GCCGUCAUG | GCCGUCAUG | AmUmGGmCCmGUC | 29942571 |
| | | GCGCCC | GCGCCCGUUG | AUmGGCGmCCCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028864 | 522 | CGAGGAUGG | CGAGGAUGG | mC*mG*mA*mGmGA | chr6:29942548- |
| | | CCGUCAUGGC | CCGUCAUGGC | UmGmGCmCGmUCA | 29942572 |
| | | GCCCC | GCCCCGUUGU | UGmGCGCmCCCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028865 | 523 | AAGGUUCCA | AAGGUUCCA | mA*mA*mG*mGmU | chr6:29944266- |
| | | UCCCCUGCAG | UCCCCUGCAG | UCmCmAUmCCmCC | 29944290 |
| | | GCCUG | GCCUGGUUG | UGCmAGGCmCUGm | |
| | | | UAGCUCCCUG | GUUGmUmAmGmCU | |
| | | | AAACCGUUG | CCCmUmGmAmAmA | |
| | | | CUACAAUAA | mCmCGUUmGmCUA | |
| | | | GGCCGUCGA | mCAAU*AAGmGmC | |
| | | | AAGAUGUGC | CmGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028866 | 524 | AGACCAGGCC | AGACCAGGCC | mA*mG*mA*mCmCA | chr6:29944268- |
| | | UGCAGGGGA | UGCAGGGGA | GmGmCCmUGmCAG | 29944292 |
| | | UGGAA | UGGAAGUUG | GGmGAUGmGAAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028867 | 525 | ACUUCUGGA | ACUUCUGGA | mA*mC*mU*mUmCU | chr6:29944274- |
| | | AGGUUCCAU | AGGUUCCAU | GmGmAAmGGmUUC | 29944298 |
| | | CCCCUG | CCCCUGGUUG | CAmUCCCmCUGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028868 | 526 | CGAUGAAGC | CGAUGAAGC | mC*mG*mA*mUmGA | chr6:29942795- |
| | | GGGGCUCCCC | GGGGCUCCCC | AmGmCGmGGmGCU | 29942819 |
| | | GCGGC | GCGGCGUUG | CCmCCGCmGGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028869 | 527 | UCCGUGUCCC | UCCGUGUCCC | mU*mC*mC*mGmUG | chr6:29942785- |
| | | GGCCCGGCCG | GGCCCGGCCG | UmCmCCmGGmCCC | 29942809 |
| | | CGGG | CGGGGUUGU | GGmCCGCmGGGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028870 | 528 | CCGUGUCCCG | CCGUGUCCCG | mC*mC*mG*mUmGU | chr6:29942786- |
| | | GCCCGGCCGC | GCCCGGCCGC | CmCmCGmGCmCCG | 29942810 |
| | | GGGG | GGGGGUUGU | GCmCGCGmGGGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028871 | 529 | CAGACGCCGA | CAGACGCCGA | mC*mA*mG*mAmCG | chr6:29942541- |
| | | GGAUGGCCG | GGAUGGCCG | CmCmGAmGGmAUG | 29942565 |
| | | UCAUG | UCAUGGUUG | GCmCGUCmAUGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028872 | 530 | CCAGACGCCG | CCAGACGCCG | mC*mC*mA*mGmAC | chr6:29942540- |
| | | AGGAUGGCC | AGGAUGGCC | GmCmCGmAGmGAU | 29942564 |
| | | GUCAU | GUCAUGUUG | GGmCCGUmCAUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028873 | 531 | AGACGCCGA | AGACGCCGA | mA*mG*mA*mCmGC | chr6:29942542- |
| | | GGAUGGCCG | GGAUGGCCG | CmGmAGmGAmUGG | 29942566 |
| | | UCAUGG | UCAUGGGUU | CCmGUCAmUGGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028874 | 532 | CUGUCGAACC | CUGUCGAACC | mC*mU*mG*mUmCG | chr6:29942838- |
| | | GCACGAACU | GCACGAACU | AmAmCCmGCmACG | 29942862 |
| | | GCGUG | GCGUGGUUG | AAmCUGCmGUGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028875 | 533 | UCGUGCGGU | UCGUGCGGU | mU*mC*mG*mUmGC | chr6:29942852- |
| | | UCGACAGCG | UCGACAGCG | GmGmUUmCGmACA | 29942876 |
| | | ACGCCG | ACGCCGGUU | GCmGACGmCCGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028876 | 534 | CCAUCCCCAU | CCAUCCCCAU | mC*mC*mA*mUmCC | chr6:29944517- |
| | | CGUGGGCAU | CGUGGGCAU | CmCmAUmCGmUGG | 29944541 |
| | | CAUUG | CAUUGGUUG | GCmAUCAmUUGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028877 | 535 | UCCUCUGGCU | UCCUCUGGCU | mU*mC*mC*mUmCU | chr6:29942858- |
| | | CGCGGCGUCG | CGCGGCGUCG | GmGmCUmCGmCGG | 29942882 |
| | | CUGU | CUGUGUUGU | CGmUCGCmUGUmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028878 | 536 | CUCUGGUUG | CUCUGGUUG | mC*mU*mC*mUmGG | chr6:29942988- |
| | | UAGUAGCCG | UAGUAGCCG | UmUmGUmAGmUAG | 29943012 |
| | | CGCAGG | CGCAGGGUU | CCmGCGCmAGGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028879 | 537 | ACCUGGGGA | ACCUGGGGA | mA*mC*mC*mUmGG | chr6:29942984- |
| | | CCCUGCGCGG | CCCUGCGCGG | GmGmACmCCmUGC | 29943008 |
| | | CUACU | CUACUGUUG | GCmGGCUmACUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028880 | 538 | CGCUCUGGU | CGCUCUGGU | mC*mG*mC*mUmCU | chr6:29942990- |
| | | UGUAGUAGC | UGUAGUAGC | GmGmUUmGUmAGU | 29943014 |
| | | CGCGCA | CGCGCAGUU | AGmCCGCmGCAmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028881 | 539 | GCUCUGGUU | GCUCUGGUU | mG*mC*mU*mCmUG | chr6:29942989- |
| | | GUAGUAGCC | GUAGUAGCC | GmUmUGmUAmGUA | 29943013 |
| | | GCGCAG | GCGCAGGUU | GCmCGCGmCAGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028882 | 540 | UAUUUUUUU | UAUUUUUUU | mU*mA*mU*mUmU | chr6:29945435- |
| | | CUAUAGUGU | CUAUAGUGU | UUmUmUCmUAmUA | 29945459 |
| | | GAGACA | GAGACAGUU | GUGmUGAGmACAm | |
| | | | GUAGCUCCCU | GUUGmUmAmGmCU | |
| | | | GAAACCGUU | CCCmUmGmAmAmA | |
| | | | GCUACAAUA | mCmCGUUmGmCUA | |
| | | | AGGCCGUCG | mCAAU*AAGmGmC | |
| | | | AAAGAUGUG | CmGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028883 | 541 | ACGCCUACGA | ACGCCUACGA | mA*mC*mG*mCmCU | chr6:29943342- |
| | | CGGCAAGGA | CGGCAAGGA | AmCmGAmCGmGCA | 29943366 |
| | | UUACA | UUACAGUUG | AGmGAUUmACAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028884 | 542 | UUUGUUCUA | UUUGUUCUA | mU*mU*mU*mGmU | chr6:29945218- |
| | | CCCCAGGCAG | CCCCAGGCAG | UCmUmACmCCmCA | 29945242 |
| | | UGACA | UGACAGUUG | GGCmAGUGmACAm | |
| | | | UAGCUCCCUG | GUUGmUmAmGmCU | |
| | | | AAACCGUUG | CCCmUmGmAmAmA | |
| | | | CUACAAUAA | mCmCGUUmGmCUA | |
| | | | GGCCGUCGA | mCAAU*AAGmGmC | |
| | | | AAGAUGUGC | CmGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028885 | 543 | UUUUGUUCU | UUUUGUUCU | mU*mU*mU*mUmG | chr6:29945217- |
| | | ACCCCAGGCA | ACCCCAGGCA | UUmCmUAmCCmCC | 29945241 |
| | | GUGAC | GUGACGUUG | AGGmCAGUmGACm | |
| | | | UAGCUCCCUG | GUUGmUmAmGmCU | |
| | | | AAACCGUUG | CCCmUmGmAmAmA | |
| | | | CUACAAUAA | mCmCGUUmGmCUA | |
| | | | GGCCGUCGA | mCAAU*AAGmGmC | |
| | | | AAGAUGUGC | CmGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028886 | 544 | AGCGACCACA | AGCGACCACA | mA*mG*mC*mGmAC | chr6:29944558- |
| | | GCUCCAGUG | GCUCCAGUG | CmAmCAmGCmUCC | 29944582 |
| | | AUCAC | AUCACGUUG | AGmUGAUmCACmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028887 | 545 | GAGCUCCGU | GAGCUCCGU | mG*mA*mG*mCmUC | chr6:29944234- |
| | | GUCCUGGGU | GUCCUGGGU | CmGmUGmUCmCUG | 29944258 |
| | | CUGGUC | CUGGUCGUU | GGmUCUGmGUCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028888 | 546 | UCCACGAGCU | UCCACGAGCU | mUmC*mC*mAmCG | chr6:29944239- |
| | | CCGUGUCCUG | CCGUGUCCUG | AmGmCUmCCmGUG | 29944263 |
| | | GGUC | GGUCGUUGU | UCmCUGGmGUCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028889 | 547 | ACGAGCUCCG | ACGAGCUCCG | mA*mC*mG*mAmGC | chr6:29944236- |
| | | UGUCCUGGG | UGUCCUGGG | UmCmCGmUGmUCC | 29944260 |
| | | UCUGG | UCUGGGUUG | UGmGGUCmUGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028890 | 548 | CGAGCUCCGU | CGAGCUCCGU | mC*mG*mA*mGmCU | chr6:29944235- |
| | | GUCCUGGGU | GUCCUGGGU | CmCmGUmGUmCCU | 29944259 |
| | | CUGGU | CUGGUGUUG | GGmGUCUmGGUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028891 | 549 | GCAGGCCUG | GCAGGCCUG | mG*mC*mA*mGmGC | chr6:29944251- |
| | | GUCUCCACGA | GUCUCCACGA | CmUmGGmUCmUCC | 29944275 |
| | | GCUCC | GCUCCGUUG | ACmGAGCmUCCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028892 | 550 | UCCCCUGCAG | UCCCCUGCAG | mU*mC*mC*mCmCU | chr6:29944257- |
| | | GCCUGGUCUC | GCCUGGUCUC | GmCmAGmGCmCUG | 29944281 |
| | | CACG | CACGGUUGU | GUmCUCCmACGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028893 | 551 | AUCCGUGUCC | AUCCGUGUCC | mA*mU*mC*mCmGU | chr6:29942784- |
| | | CGGCCCGGCC | CGGCCCGGCC | GmUmCCmCGmGCC | 29942808 |
| | | GCGG | GCGGGUUGU | CGmGCCGmCGGmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028894 | 552 | CCUUCCCGUU | CCUUCCCGUU | mC*mC*mU*mUmCC | chr6:29943495- |
| | | CUCCAGGUA | CUCCAGGUA | CmGmUUmCUmCCA | 29943519 |
| | | UCUGC | UCUGCGUUG | GGmUAUCmUGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028895 | 553 | CCGUUCUCCA | CCGUUCUCCA | mC*mC*mG*mUmUC | chr6:29943490- |
| | | GGUAUCUGC | GGUAUCUGC | UmCmCAmGGmUAU | 29943514 |
| | | GGAGC | GGAGCGUUG | CUmGCGGmAGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028896 | 554 | ACCUGCCAUG | ACCUGCCAUG | mA*mC*mC*mUmGC | chr6:29944345- |
| | | UGCAGCAUG | UGCAGCAUG | CmAmUGmUGmCAG | 29944369 |
| | | AGGGU | AGGGUGUUG | CAmUGAGmGGUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028897 | 555 | CACCUGCCAU | CACCUGCCAU | mC*mA*mC*mCmUG | chr6:29944344- |
| | | GUGCAGCAU | GUGCAGCAU | CmCmAUmGUmGCA | 29944368 |
| | | GAGGG | GAGGGGUUG | GCmAUGAmGGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028898 | 556 | ACCUGGCAGC | ACCUGGCAGC | mA*mC*mC*mUmGG | chr6:29944219- |
| | | GGGAUGGGG | GGGAUGGGG | CmAmGCmGGmGAU | 29944243 |
| | | AGGAC | AGGACGUUG | GGmGGAGmGACmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028899 | 557 | CACUGACCUG | CACUGACCUG | mC*mA*mC*mUmGA | chr6:29944214- |
| | | GCAGCGGGA | GCAGCGGGA | CmCmUGmGCmAGC | 29944238 |
| | | UGGGG | UGGGGGUUG | GGmGAUGmGGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028900 | 558 | CCUGGCAGCG | CCUGGCAGCG | mC*mC*mU*mGmGC | chr6:29944220- |
| | | GGAUGGGGA | GGAUGGGGA | AmGmCGmGGmAUG | 29944244 |
| | | GGACC | GGACCGUUG | GGmGAGGmACCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028901 | 559 | CCAUCUCAGG | CCAUCUCAGG | mC*mC*mA*mUmCU | chr6:29944366- |
| | | GUGAGGGGC | GUGAGGGGC | CmAmGGmGUmGAG | 29944390 |
| | | UUGGG | UUGGGGUUG | GGmGCUUmGGGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028902 | 560 | UGGCCGUCA | UGGCCGUCA | mU*mG*mG*mCmCG | chr6:29942554- |
| | | UGGCGCCCCG | UGGCGCCCCG | UmCmAUmGGmCGC | 29942578 |
| | | AACCC | AACCCGUUG | CCmCGAAmCCCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028903 | 561 | AUGUCCGCCG | AUGUCCGCCG | mA*mU*mG*mUmCC | chr6:29943379- |
| | | CGGUCCAAG | CGGUCCAAG | GmCmCGmCGmGUC | 29943403 |
| | | AGCGC | AGCGCGUUG | CAmAGAGmCGCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028904 | 562 | GGUACCCGU | GGUACCCGU | mG*mG*mU*mAmCC | chr6:29943518- |
| | | GCGCUGCAGC | GCGCUGCAGC | CmGmUGmCGmCUG | 29943542 |
| | | GUCUC | GUCUCGUUG | CAmGCGUmCUCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028905 | 563 | GGGAAGGAG | GGGAAGGAG | mG*mG*mG*mAmA | chr6:29943518- |
| | | ACGCUGCAGC | ACGCUGCAGC | GGmAmGAmCGmCU | 29943542 |
| | | GCACG | GCACGGUUG | GCAmGCGCmACGm | |
| | | | UAGCUCCCUG | GUUGmUmAmGmCU | |
| | | | AAACCGUUG | CCCmUmGmAmAmA | |
| | | | CUACAAUAA | mCmCGUUmGmCUA | |
| | | | GGCCGUCGA | mCAAU*AAGmGmC | |
| | | | AAGAUGUGC | CmGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028906 | 564 | GCGGGCGCCG | GCGGGCGCCG | mG*mC*mG*mGmGC | chr6:29942895- |
| | | UGGAUAGAG | UGGAUAGAG | GmCmCGmUGmGAU | 29942919 |
| | | CAGGA | CAGGAGUUG | AGmAGCAmGGAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028907 | 565 | UCCUGCUCUA | UCCUGCUCUA | mU*mC*mC*mUmGC | chr6:29942889- |
| | | UCCACGGCGC | UCCACGGCGC | UmCmUAmUCmCAC | 29942913 |
| | | CCGC | CCGCGUUGU | GGmCGCCmCGCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028908 | 566 | CCCCUGGUAC | CCCCUGGUAC | mC*mC*mC*mCmUG | chr6:29943523- |
| | | CCGUGCGCUG | CCGUGCGCUG | GmUmACmCCmGUG | 29943547 |
| | | CAGC | CAGCGUUGU | CGmCUGCmAGCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028909 | 567 | UGGUACCCG | UGGUACCCG | mU*mG*mG*mUmAC | chr6:29943519- |
| | | UGCGCUGCA | UGCGCUGCA | CmCmGUmGCmGCU | |
| | | GCGUCU | GCGUCUGUU | GCmAGCGmUCUmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | 29943543 |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028910 | 568 | CAUCUCAGG | CAUCUCAGG | mC*mA*mU*mCmUC | chr6:29944365- |
| | | GUGAGGGGC | GUGAGGGGC | AmGmGGmUGmAGG | 29944389 |
| | | UUGGGC | UUGGGCGUU | GGmCUUGmGGCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028911 | 569 | ACGCAGUUC | ACGCAGUUC | mA*mC*mG*mCmAG | chr6:29942845- |
| | | GUGCGGUUC | GUGCGGUUC | UmUmCGmUGmCGG | 29942869 |
| | | GACAGC | GACAGCGUU | UUmCGACmAGCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028912 | 570 | CGCCCAGGUC | CGCCCAGGUC | mC*mG*mC*mCmCA | chr6:29942595- |
| | | UGGGUCAGG | UGGGUCAGG | GmGmUCmUGmGGU | 29942619 |
| | | GCCAG | GCCAGGUUG | CAmGGGCmCAGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028913 | 571 | CACUCACCCG | CACUCACCCG | mC*mA*mC*mUmCA | chr6:29942609- |
| | | CCCAGGUCUG | CCCAGGUCUG | CmCmCGmCCmCAG | 29942633 |
| | | GGUC | GGUCGUUGU | GUmCUGGmGUCmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028914 | 572 | CCGCCCAGGU | CCGCCCAGGU | mC*mC*mG*mCmCC | chr6:29942596- |
| | | CUGGGUCAG | CUGGGUCAG | AmGmGUmCUmGGG | 29942620 |
| | | GGCCA | GGCCAGUUG | UCmAGGGmCCAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028915 | 573 | CCCGCCCAGG | CCCGCCCAGG | mC*mC*mC*mGmCC | chr6:29942597- |
| | | UCUGGGUCA | UCUGGGUCA | CmAmGGmUCmUGG | 29942621 |
| | | GGGCC | GGGCCGUUG | GUmCAGGmGCCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028916 | 574 | CAGCGACGCC | CAGCGACGCC | mC*mA*mG*mCmGA | chr6:29942865- |
| | | GCGAGCCAG | GCGAGCCAG | CmGmCCmGCmGAG | 29942889 |
| | | AGGAU | AGGAUGUUG | CCmAGAGmGAUmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028917 | 575 | CGCGAGCCAG | CGCGAGCCAG | mC*mG*mC*mGmAG | chr6:29942874- |
| | | AGGAUGGAG | AGGAUGGAG | CmCmAGmAGmGAU | 29942898 |
| | | CCGCG | CCGCGGUUG | GGmAGCCmGCGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028918 | 576 | GCUCUAUCCA | GCUCUAUCCA | mG*mC*mU*mCmUA | chr6:29942891- |
| | | CGGCGCCCGC | CGGCGCCCGC | UmCmCAmCGmGCG | 29942915 |
| | | GGCU | GGCUGUUGU | CCmCGCGmGCUmG | |
| | | | AGCUCCCUGA | UUGmUmAmGmCUC | |
| | | | AACCGUUGC | CCmUmGmAmAmAm | |
| | | | UACAAUAAG | CmCGUUmGmCUAm | |
| | | | GCCGUCGAA | CAAU*AAGmGmCC | |
| | | | AGAUGUGCC | mGmUmCmGmAmA | |
| | | | GCAACGCUCU | mAmGmAmUGUGCm | |
| | | | GCCUUCUGGC | CGmCAAmCGCUCU | |
| | | | AUCGUU | mGmCCmUmUmCmU | |
| | | | | GGCAUCG*mU*mU | |
| G028919 | 577 | CACAUCAGA | CACAUCAGA | mC*mA*mC*mAmUC | chr6:29945232- |
| | | GCCCUGGGCA | GCCCUGGGCA | AmGmAGmCCmCUG | 29945256 |
| | | CUGUC | CUGUCGUUG | GGmCACUmGUCmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028920 | 578 | CAGACCCAGG | CAGACCCAGG | mC*mA*mG*mAmCC | chr6:29944243- |
| | | ACACGGAGC | ACACGGAGC | CmAmGGmACmACG | 29944267 |
| | | UCGUG | UCGUGGUUG | GAmGCUCmGUGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028921 | 579 | CCAGGACACG | CCAGGACACG | mC*mC*mA*mGmGA | chr6:29944248- |
| | | GAGCUCGUG | GAGCUCGUG | CmAmCGmGAmGCU | 29944272 |
| | | GAGAC | GAGACGUUG | CGmUGGAmGACmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028922 | 580 | CUCUGGGAA | CUCUGGGAA | mC*mU*mC*mUmGG | chr6:29944471- |
| | | AAGAGGGGA | AAGAGGGGA | GmAmAAmAGmAGG | 29944495 |
| | | AGGUGA | AGGUGAGUU | GGmAAGGmUGAmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028923 | 581 | UCUGGGAAA | UCUGGGAAA | mU*mC*mU*mGmGG | chr6:29944470- |
| | | AGAGGGGAA | AGAGGGGAA | AmAmAAmGAmGGG | 29944494 |
| | | GGUGAG | GGUGAGGUU | GAmAGGUmGAGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028924 | 582 | AGACGCUGC | AGACGCUGC | mA*mG*mA*mCmGC | chr6:29943525- |
| | | AGCGCACGG | AGCGCACGG | UmGmCAmGCmGCA | 29943549 |
| | | GUACCA | GUACCAGUU | CGmGGUAmCCAmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028925 | 583 | UCCUUUUCU | UCCUUUUCU | mU*mC*mC*mUmUU | Unavailable. |
| | | AUCUGUGGG | AUCUGUGGG | UmCmUAmUCmUGU | Imperfect |
| | | AAGAAA | AAGAAAGUU | GGmGAAGmAAAmG | alignment to |
| | | | GUAGCUCCCU | UUGmUmAmGmCUC | human genome. |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028926 | 584 | CACUGCCUGG | CACUGCCUGG | mC*mA*mC*mUmGC | chr6:29945209- |
| | | GGUAGAACA | GGUAGAACA | CmUmGGmGGmUAG | 29945233 |
| | | AAAAC | AAAACGUUG | AAmCAAAmAACmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028927 | 585 | ACAACCAGA | ACAACCAGA | mA*mC*mA*mAmCC | chr6:29943008- |
| | | GCGAGGCCG | GCGAGGCCG | AmGmAGmCGmAGG | 29943032 |
| | | GUGAGU | GUGAGUGUU | CCmGGUGmAGUmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028928 | 586 | CUACAACCAG | CUACAACCAG | mC*mU*mA*mCmAA | chr6:29943006- |
| | | AGCGAGGCC | AGCGAGGCC | CmCmAGmAGmCGA | 29943030 |
| | | GGUGA | GGUGAGUUG | GGmCCGGmUGAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028929 | 587 | UACAACCAG | UACAACCAG | mU*mA*mC*mAmAC | chr6:29943007- |
| | | AGCGAGGCC | AGCGAGGCC | CmAmGAmGCmGAG | 29943031 |
| | | GGUGAG | GGUGAGGUU | GCmCGGUmGAGmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028930 | 588 | CCAGAGCGA | CCAGAGCGA | mC*mC*mA*mGmAG | chr6:29943012- |
| | | GGCCGGUGA | GGCCGGUGA | CmGmAGmGCmCGG | 29943036 |
| | | GUGACC | GUGACCGUU | UGmAGUGmACCmG | |
| | | | GUAGCUCCCU | UUGmUmAmGmCUC | |
| | | | GAAACCGUU | CCmUmGmAmAmAm | |
| | | | GCUACAAUA | CmCGUUmGmCUAm | |
| | | | AGGCCGUCG | CAAU*AAGmGmCC | |
| | | | AAAGAUGUG | mGmUmCmGmAmA | |
| | | | CCGCAACGCU | mAmGmAmUGUGCm | |
| | | | CUGCCUUCUG | CGmCAAmCGCUCU | |
| | | | GCAUCGUU | mGmCCmUmUmCmU | |
| | | | | GGCAUCG*mU*mU | |
| G028933 | 589 | CCAUCCCGCU | CCAUCCCGCU | mC*mC*mA*mUmCC | chr6:29944206- |
| | | GCCAGGUCA | GCCAGGUCA | CmGmCUmGCmCAG | 29944230 |
| | | GUGUG | GUGUGGUUG | GUmCAGUmGUGmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |
| G028934 | 590 | GUAUCUGCG | GUAUCUGCG | mG*mU*mA*mUmCU | chr6:29943479- |
| | | GAGCCACUCC | GAGCCACUCC | GmCmGGmAGmCCA | 29943503 |
| | | ACGCA | ACGCAGUUG | CUmCCACmGCAmG | |
| | | | UAGCUCCCUG | UUGmUmAmGmCUC | |
| | | | AAACCGUUG | CCmUmGmAmAmAm | |
| | | | CUACAAUAA | CmCGUUmGmCUAm | |
| | | | GGCCGUCGA | CAAU*AAGmGmCC | |
| | | | AAGAUGUGC | mGmUmCmGmAmA | |
| | | | CGCAACGCUC | mAmGmAmUGUGCm | |
| | | | UGCCUUCUG | CGmCAAmCGCUCU | |
| | | | GCAUCGUU | mGmCCmUmUmCmU | |
| | | | | GGCAUCG*mU*mU | |

In some embodiments, the HLA-A guide RNA comprises a guide sequence selected from any one of SEQ ID NOs: 301-590. In some embodiments, the HLA-A guide RNA comprises a guide sequence that is at least 17, 18, 19, 20, 21, 22, 23, or 24 contiguous nucleotides of a sequence selected from SEQ ID NOs: 301-590. In some embodiments, the HLA-A guide RNA comprises a guide sequence that is at least 95%, 90%, 85%, 80%, 75%, or 70% identical to a sequence selected from SEQ ID NOs: 301-590. In some embodiments, the HLA-A guide RNA comprises a guide sequence that is at least 95% identical to a sequence selected from SEQ ID NOs: 301-590.

In some embodiments, the HLA-A gRNA comprises a guide sequence selected from any one of SEQ ID NOs: 301-395. In some embodiments, the HLA-A gRNA comprises a guide sequence selected from any one of SEQ ID NOs: 307, 313-318, 322, 326, 331, 333, 337-341, 343, 345, 347, 357, 359, 362, 366, 387. In some embodiments, the HLA-A gRNA comprises a guide sequence selected from any one of SEQ ID NOs: 313-318, 326, 337-339, 341, 343, 345, 362. In some embodiments, the HLA-A gRNA comprises a guide sequence selected from any one of SEQ ID NOs: 313-318. In some embodiments, the HLA-A gRNA comprises a guide sequence selected from any one of SEQ ID NOs: 313-317. n some embodiments, the HLA-A gRNA comprises a guide sequence selected from any one of SEQ ID NOs: 337-339, 341, 343, and 345. In some embodiments, the HLA-A gRNA comprises a guide sequence selected from any one of SEQ ID NOs: 337-339. In some embodiments, the HLA-A gRNA comprises a guide sequence selected from any one of SEQ ID NOs: 523, 565, 571, 576, 580, 581.

In some embodiments, the HLA-A guide RNA comprises a guide sequence that comprises at least 10 contiguous nucleotides ±10 nucleotides of a genomic coordinate listed in Tables 4-7. As used herein, at least 10 contiguous nucleotides ±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Tables 4-7. For example, an HLA-A guide RNA may comprise 10 contiguous nucleotides within the genomic coordinates chr6:29942864 to chr6: 29942903 or chr6:29943528 to chr6:29943609, including the boundary nucleotides of these ranges. In some embodiments, the HLA-A guide RNA comprises a guide sequence that is at least 17, 18, 19, or 20 contiguous nucleotides of a sequence that comprises 10 contiguous nucleotides ±10 nucleotides of a genomic coordinate listed in Tables 4, 5B and 6, or a guide sequence that is complementary to at least 17, 18, 19, 20, 21, 22, 23, or 24 contiguous nucleotides of a sequence that comprises 10 contiguous nucleotides ±10 nucleotides of a genomic coordinate listed in Tables 5A and 7. In some embodiments, the HLA-A guide RNA comprises a guide sequence that is at least 95%, 90%, or 85% identical to a sequence selected from a sequence that is 17, 18, 19, or 20 contiguous nucleotides of a sequence that comprises 10 contiguous nucleotides ±10 nucleotides of a genomic coordinate listed in Tables 4, 5B and 6, or a guide sequence that is complementary to at least 17, 18, 19, 20, 21, 22, 23, or 24 contiguous nucleotides of a sequence that comprises 10 contiguous nucleotides ±10 nucleotides of a genomic coordinate listed in Tables 5A and 7.

In some embodiments, the guide RNA comprises a guide sequence that comprises at least 15 contiguous nucleotides ±10 nucleotides of a genomic coordinate listed in Tables 4-7. In some embodiments, the HLA-A guide RNA comprises a guide sequence that comprises at least 20 contiguous nucleotides ±10 nucleotides of a genomic coordinate listed in Tables 4-7.

In some embodiments, the HLA-A guide RNA comprises SEQ ID NO: 301. In some embodiments, the HLA-A guide RNA comprises SEQ ID NO: 302. In some embodiments, the HLA-A guide RNA comprises SEQ ID NO: 303. In some embodiments, the HLA-A guide RNA comprises SEQ ID NO: 304. In some embodiments, the HLA-A guide RNA comprises SEQ ID NO: 305. In some embodiments, the HLA-A guide RNA comprises SEQ ID NO: 306. In some embodiments, the HLA-A guide RNA comprises SEQ ID NO: 307. In some embodiments, the HLA-A guide RNA comprises SEQ ID NO: 308. In some embodiments, the HLA-A guide RNA comprises SEQ ID NO: 309. In some embodiments, the HLA-A guide RNA comprises SEQ ID NO: 310. In some embodiments, the HLA-A guide RNA comprises SEQ ID NO: 311. In some embodiments, the HLA-A guide RNA comprises SEQ ID NO: 312. In some embodiments, the HLA-A guide RNA comprises SEQ ID NO: 313. In some embodiments, the HLA-A guide RNA comprises SEQ ID NO: 314. In some embodiments, the HLA-A guide RNA comprises SEQ ID NO: 315. In some embodiments, the HLA-A guide RNA comprises SEQ ID NO: 316. In some embodiments, the HLA-A guide RNA comprises SEQ ID NO: 317. In some embodiments, the HLA-A guide RNA comprises SEQ ID NO: 318. In some embodiments, the HLA-A guide RNA comprises SEQ ID NO: 319. In some embodiments, the HLA-A guide RNA comprises SEQ ID NO: 320. In some embodiments, the HLA-A guide RNA comprises SEQ ID NO: 321. In some embodiments, the HLA-A guide RNA comprises SEQ ID NO: 322. In some embodiments, the HLA-A guide RNA comprises SEQ ID NO: 323. In some embodiments, the HLA-A guide RNA comprises SEQ ID NO: 324. In some embodiments, the HLA-A guide RNA comprises SEQ ID NO: 325. In some embodiments, the HLA-A guide RNA comprises SEQ ID NO: 326. In some embodiments, the HLA-A guide RNA comprises SEQ ID NO: 327. In some embodiments, the HLA-A guide RNA comprises SEQ ID NO: 328. In some embodiments, the HLA-A guide RNA comprises SEQ ID NO: 329. In some embodiments, the HLA-A guide RNA comprises SEQ ID NO: 330. In some embodiments, the HLA-A guide RNA comprises SEQ ID NO: 331. In some embodiments, the HLA-A guide RNA comprises SEQ ID NO: 332. In some embodiments, the HLA-A guide RNA comprises SEQ ID NO: 333. In some embodiments, the HLA-A guide RNA comprises SEQ ID NO: 334. In some embodiments, the HLA-A guide RNA comprises SEQ ID NO: 335. In some embodiments, the HLA-A guide RNA comprises SEQ ID NO: 336. In some embodiments, the HLA-A guide RNA comprises SEQ ID NO: 337. In some embodiments, the HLA-A guide RNA comprises SEQ ID NO: 338. In some embodiments, the HLA-A guide RNA comprises SEQ ID NO: 339. In some embodiments, the HLA-A guide RNA comprises SEQ ID NO: 340. In some embodiments, the HLA-A guide RNA comprises SEQ ID NO: 341. In some embodiments, the HLA-A guide RNA comprises SEQ ID NO: 342. In some embodiments, the HLA-A guide RNA comprises SEQ ID NO: 343. In some embodiments, the HLA-A guide RNA comprises SEQ ID NO: 344. In some embodiments, the HLA-A guide RNA comprises SEQ ID NO: 345. In some embodiments, the HLA-A guide RNA comprises SEQ ID NO: 346. In some embodiments, the HLA-A guide RNA comprises SEQ ID NO: 347. In some embodiments, the HLA-A guide RNA comprises SEQ ID NO: 348. In some embodiments, the HLA-A guide RNA comprises SEQ ID NO: 349. In some embodiments, the HLA-A guide RNA comprises SEQ ID NO: 350. In some embodiments, the HLA-A guide RNA comprises SEQ ID NO: 351. In some embodiments, the HLA-A guide RNA comprises SEQ ID NO: 352. In some embodiments, the HLA-A guide RNA comprises SEQ ID NO: 353. In some embodiments, the HLA-A guide RNA comprises SEQ ID NO: 354. In some embodiments, the HLA-A guide RNA comprises SEQ ID NO: 355. In some embodiments, the HLA-A guide RNA comprises SEQ ID NO: 356. In some embodiments, the HLA-A guide RNA comprises SEQ ID NO: 357. In some embodiments, the HLA-A guide RNA comprises SEQ ID NO: 358. In some embodiments, the HLA-A guide RNA comprises SEQ ID NO: 359. In some embodiments, the HLA-A guide RNA comprises SEQ ID NO: 360. In some embodiments, the HLA-A guide RNA comprises SEQ ID NO: 361. In some embodiments, the HLA-A guide RNA comprises SEQ ID NO: 362. In some embodiments, the HLA-A guide RNA comprises SEQ ID NO: 363. In some embodiments, the HLA-A guide RNA comprises SEQ ID NO: 364. In some embodiments, the HLA-A guide RNA comprises SEQ ID NO: 365. In some embodiments, the HLA-A guide RNA comprises SEQ ID NO: 366. In some embodiments, the HLA-A guide RNA comprises SEQ ID NO: 367. In some embodiments, the HLA-A guide RNA comprises SEQ ID NO: 368. In some embodiments, the HLA-A guide RNA comprises SEQ ID NO: 369. In some embodiments, the HLA-A guide RNA comprises SEQ ID NO: 370. In some embodiments, the HLA-A guide RNA comprises SEQ ID NO: 371. In some embodiments, the HLA-A guide RNA comprises SEQ ID NO: 372. In some embodiments, the HLA-A guide RNA comprises SEQ ID NO: 373. In some embodiments, the HLA-A guide RNA comprises SEQ ID NO: 374. In some embodiments, the HLA-A guide RNA comprises SEQ ID NO: 375. In some embodiments, the HLA-A guide RNA comprises SEQ ID NO: 376. In some embodiments, the HLA-A guide RNA comprises SEQ ID NO: 377. In some embodiments, the HLA-A guide RNA comprises SEQ ID NO: 378. In some embodiments, the HLA-A guide RNA comprises SEQ ID NO: 379. In some embodiments, the HLA-A guide RNA comprises SEQ ID NO: 380. In some embodiments, the HLA-A guide RNA comprises SEQ ID NO: 381. In some embodiments, the HLA-A guide RNA comprises SEQ ID NO: 382. In some embodiments, the HLA-A guide RNA comprises SEQ ID NO: 383. In some embodiments, the HLA-A guide RNA comprises SEQ ID NO: 384. In some embodiments, the HLA-A guide RNA comprises SEQ ID NO: 385. In some embodiments, the HLA-A guide RNA comprises SEQ ID NO: 386. In some embodiments, the HLA-A guide RNA comprises SEQ ID NO: 387. In some embodiments, the HLA-A guide RNA comprises SEQ ID NO: 388. In some embodiments, the HLA-A guide RNA comprises SEQ ID NO: 389. In some embodiments, the HLA-A guide RNA comprises SEQ ID NO: 390. In some embodiments, the HLA-A guide RNA comprises SEQ ID NO: 391. In some embodiments, the HLA-A guide RNA comprises SEQ ID NO: 392. In some embodiments, the HLA-A guide RNA comprises SEQ ID NO: 393. In some embodiments, the HLA-A guide RNA comprises SEQ ID NO: 394. In some embodiments, the HLA-A guide RNA comprises SEQ ID NO: 395. In some embodiments, the HLA-A guide RNA comprises SEQ ID NO: 396. In some embodiments, the HLA-A guide RNA comprises SEQ ID NO: 397. In some embodiments, the HLA-A guide RNA comprises SEQ ID NO: 398. In some embodiments, the HLA-A guide RNA comprises SEQ ID NO: 399. In some embodiments, the HLA-A guide RNA comprises SEQ ID NO: 400. In some embodiments, the HLA-A guide RNA comprises SEQ ID NO: 401. In some embodiments, the HLA-A guide RNA comprises SEQ ID NO: 402. In some embodiments, the HLA-A guide RNA comprises SEQ ID NO: 403. In some embodiments, the HLA-A guide RNA comprises SEQ ID NO: 404. In some embodiments, the HLA-A guide RNA comprises SEQ ID NO: 405. In some embodiments, the HLA-A guide RNA comprises SEQ ID NO: 406. In some embodiments, the HLA-A guide RNA comprises SEQ ID NO: 407. In some embodiments, the HLA-A guide RNA comprises SEQ ID NO: 408. In some embodiments, the HLA-A guide RNA comprises SEQ ID NO: 409. In some embodiments, the HLA-A guide RNA comprises SEQ ID NO: 410. In some embodiments, the HLA-A guide RNA comprises SEQ ID NO: 411. In some embodiments, the HLA-A guide RNA comprises SEQ ID NO: 412. In some embodiments, the HLA-A guide RNA comprises SEQ ID NO: 413. In some embodiments, the HLA-A guide RNA comprises SEQ ID NO: 414. In some embodiments, the HLA-A guide RNA comprises SEQ ID NO: 415. In some embodiments, the HLA-A guide RNA comprises SEQ ID NO: 416. In some embodiments, the HLA-A guide RNA comprises SEQ ID NO: 417. In some embodiments, the HLA-A guide RNA comprises SEQ ID NO: 418. In some embodiments, the HLA-A guide RNA comprises SEQ ID NO: 419. In some embodiments, the HLA-A guide RNA comprises SEQ ID NO: 420. In some embodiments, the HLA-A guide RNA comprises SEQ ID NO: 421. In some embodiments, the HLA-A guide RNA comprises SEQ ID NO: 422. In some embodiments, the HLA-A guide RNA comprises SEQ ID NO: 423. In some embodiments, the HLA-A guide RNA comprises SEQ ID NO: 424. In some embodiments, the HLA-A guide RNA comprises SEQ ID NO: 425. In some embodiments, the HLA-A guide RNA comprises SEQ ID NO: 426. In some embodiments, the HLA-A guide RNA comprises SEQ ID NO: 427. In some embodiments, the HLA-A guide RNA comprises SEQ ID NO: 428. In some embodiments, the HLA-A guide RNA comprises SEQ ID NO: 429. In some embodiments, the HLA-A guide RNA comprises SEQ ID NO: 430. In some embodiments, the HLA-A guide RNA comprises SEQ ID NO: 431. In some embodiments, the HLA-A guide RNA comprises SEQ ID NO: 432. In some embodiments, the HLA-A guide RNA comprises SEQ ID NO: 433. In some embodiments, the HLA-A guide RNA comprises SEQ ID NO: 434. In some embodiments, the HLA-A guide RNA comprises SEQ ID NO: 435. In some embodiments, the HLA-A guide RNA comprises SEQ ID NO: 436. In some embodiments, the HLA-A guide RNA comprises SEQ ID NO: 437. In some embodiments, the HLA-A guide RNA comprises SEQ ID NO: 438. In some embodiments, the HLA-A guide RNA comprises SEQ ID NO: 439. In some embodiments, the HLA-A guide RNA comprises SEQ ID NO: 440. In some embodiments, the HLA-A guide RNA comprises SEQ ID NO: 441. In some embodiments, the HLA-A guide RNA comprises SEQ ID NO: 442. In some embodiments, the HLA-A guide RNA comprises SEQ ID NO: 443. In some embodiments, the HLA-A guide RNA comprises SEQ ID NO: 444. In some embodiments, the HLA-A guide RNA comprises SEQ ID NO: 445. In some embodiments, the HLA-A guide RNA comprises SEQ ID NO: 446. In some embodiments, the HLA-A guide RNA comprises SEQ ID NO: 447. In some embodiments, the HLA-A guide RNA comprises SEQ ID NO: 448. In some embodiments, the HLA-A guide RNA comprises SEQ ID NO: 449. In some embodiments, the HLA-A guide RNA comprises SEQ ID NO: 450. In some embodiments, the HLA-A guide RNA comprises SEQ ID NO: 451. In some embodiments, the HLA-A guide RNA comprises SEQ ID NO: 452. In some embodiments, the HLA-A guide RNA comprises SEQ ID NO: 453. In some embodiments, the HLA-A guide RNA comprises SEQ ID NO: 454. In some embodiments, the HLA-A guide RNA comprises SEQ ID NO: 455. In some embodiments, the HLA-A guide RNA comprises SEQ ID NO: 456. In some embodiments, the HLA-A guide RNA comprises SEQ ID NO: 457. In some embodiments, the HLA-A guide RNA comprises SEQ ID NO: 458. In some embodiments, the HLA-A guide RNA comprises SEQ ID NO: 459. In some embodiments, the HLA-A guide RNA comprises SEQ ID NO: 460. In some embodiments, the HLA-A guide RNA comprises SEQ ID NO: 461. In some embodiments, the HLA-A guide RNA comprises SEQ ID NO: 462. In some embodiments, the HLA-A guide RNA comprises SEQ ID NO: 463. In some embodiments, the HLA-A guide RNA comprises SEQ ID NO: 464. In some embodiments, the HLA-A guide RNA comprises SEQ ID NO: 465. In some embodiments, the HLA-A guide RNA comprises SEQ ID NO: 466. In some embodiments, the HLA-A guide RNA comprises SEQ ID NO: 467. In some embodiments, the HLA-A guide RNA comprises SEQ ID NO: 468. In some embodiments, the HLA-A guide RNA comprises SEQ ID NO: 469. In some embodiments, the HLA-A guide RNA comprises SEQ ID NO: 470. In some embodiments, the HLA-A guide RNA comprises SEQ ID NO: 471. In some embodiments, the HLA-A guide RNA comprises SEQ ID NO: 472. In some embodiments, the HLA-A guide RNA comprises SEQ ID NO: 473. In some embodiments, the HLA-A guide RNA comprises SEQ ID NO: 474. In some embodiments, the HLA-A guide RNA comprises SEQ ID NO: 475. In some embodiments, the HLA-A guide RNA comprises SEQ ID NO: 476. In some embodiments, the HLA-A guide RNA comprises SEQ ID NO: 477. In some embodiments, the HLA-A guide RNA comprises SEQ ID NO: 478. In some embodiments, the HLA-A guide RNA comprises SEQ ID NO: 479. In some embodiments, the HLA-A guide RNA comprises SEQ ID NO: 480. In some embodiments, the HLA-A guide RNA comprises SEQ ID NO: 481. In some embodiments, the HLA-A guide RNA comprises SEQ ID NO: 482. In some embodiments, the HLA-A guide RNA comprises SEQ ID NO: 483. In some embodiments, the HLA-A guide RNA comprises SEQ ID NO: 484. In some embodiments, the HLA-A guide RNA comprises SEQ ID NO: 485. In some embodiments, the HLA-A guide RNA comprises SEQ ID NO: 486. In some embodiments, the HLA-A guide RNA comprises SEQ ID NO: 487. In some embodiments, the HLA-A guide RNA comprises SEQ ID NO: 488. In some embodiments, the HLA-A guide RNA comprises SEQ ID NO: 489. In some embodiments, the HLA-A guide RNA comprises SEQ ID NO: 490. In some embodiments, the HLA-A guide RNA comprises SEQ ID NO: 491. In some embodiments, the HLA-A guide RNA comprises SEQ ID NO: 492. In some embodiments, the HLA-A guide RNA comprises SEQ ID NO: 493. In some embodiments, the HLA-A guide RNA comprises SEQ ID NO: 494. In some embodiments, the HLA-A guide RNA comprises SEQ ID NO: 495. In some embodiments, the HLA-A guide RNA comprises SEQ ID NO: 496. In some embodiments, the HLA-A guide RNA comprises SEQ ID NO: 497. In some embodiments, the HLA-A guide RNA comprises SEQ ID NO: 498. In some embodiments, the HLA-A guide RNA comprises SEQ ID NO: 499. In some embodiments, the HLA-A guide RNA comprises SEQ ID NO: 500. In some embodiments, the HLA-A guide RNA comprises SEQ ID NO: 501. In some embodiments, the HLA-A guide RNA comprises SEQ ID NO: 502. In some embodiments, the HLA-A guide RNA comprises SEQ ID NO: 503. In some embodiments, the HLA-A guide RNA comprises SEQ ID NO: 504. In some embodiments, the HLA-A guide RNA comprises SEQ ID NO: 505. In some embodiments, the HLA-A guide RNA comprises SEQ ID NO: 506. In some embodiments, the HLA-A guide RNA comprises SEQ ID NO: 507. In some embodiments, the HLA-A guide RNA comprises SEQ ID NO: 508. In some embodiments, the HLA-A guide RNA comprises SEQ ID NO: 509. In some embodiments, the HLA-A guide RNA comprises SEQ ID NO: 510. In some embodiments, the HLA-A guide RNA comprises SEQ ID NO: 511. In some embodiments, the HLA-A guide RNA comprises SEQ ID NO: 512. In some embodiments, the HLA-A guide RNA comprises SEQ ID NO: 513. In some embodiments, the HLA-A guide RNA comprises SEQ ID NO: 514. In some embodiments, the HLA-A guide RNA comprises SEQ ID NO: 515. In some embodiments, the HLA-A guide RNA comprises SEQ ID NO: 516. In some embodiments, the HLA-A guide RNA comprises SEQ ID NO: 517. In some embodiments, the HLA-A guide RNA comprises SEQ ID NO: 518. In some embodiments, the HLA-A guide RNA comprises SEQ ID NO: 519. In some embodiments, the HLA-A guide RNA comprises SEQ ID NO: 520. In some embodiments, the HLA-A guide RNA comprises SEQ ID NO: 521. In some embodiments, the HLA-A guide RNA comprises SEQ ID NO: 522. In some embodiments, the HLA-A guide RNA comprises SEQ ID NO: 523. In some embodiments, the HLA-A guide RNA comprises SEQ ID NO: 524. In some embodiments, the HLA-A guide RNA comprises SEQ ID NO: 525. In some embodiments, the HLA-A guide RNA comprises SEQ ID NO: 526. In some embodiments, the HLA-A guide RNA comprises SEQ ID NO: 527. In some embodiments, the HLA-A guide RNA comprises SEQ ID NO: 528. In some embodiments, the HLA-A guide RNA comprises SEQ ID NO: 529. In some embodiments, the HLA-A guide RNA comprises SEQ ID NO: 530. In some embodiments, the HLA-A guide RNA comprises SEQ ID NO: 531. In some embodiments, the HLA-A guide RNA comprises SEQ ID NO: 532. In some embodiments, the HLA-A guide RNA comprises SEQ ID NO: 533. In some embodiments, the HLA-A guide RNA comprises SEQ ID NO: 534. In some embodiments, the HLA-A guide RNA comprises SEQ ID NO: 535. In some embodiments, the HLA-A guide RNA comprises SEQ ID NO: 536. In some embodiments, the HLA-A guide RNA comprises SEQ ID NO: 537. In some embodiments, the HLA-A guide RNA comprises SEQ ID NO: 538. In some embodiments, the HLA-A guide RNA comprises SEQ ID NO: 540. In some embodiments, the HLA-A guide RNA comprises SEQ ID NO: 541. In some embodiments, the HLA-A guide RNA comprises SEQ ID NO: 542. In some embodiments, the HLA-A guide RNA comprises SEQ ID NO: 543. In some embodiments, the HLA-A guide RNA comprises SEQ ID NO: 544. In some embodiments, the HLA-A guide RNA comprises SEQ ID NO: 545. In some embodiments, the HLA-A guide RNA comprises SEQ ID NO: 546. In some embodiments, the HLA-A guide RNA comprises SEQ ID NO: 547. In some embodiments, the HLA-A guide RNA comprises SEQ ID NO: 548. In some embodiments, the HLA-A guide RNA comprises SEQ ID NO: 549. In some embodiments, the HLA-A guide RNA comprises SEQ ID NO: 550. In some embodiments, the HLA-A guide RNA comprises SEQ ID NO: 551. In some embodiments, the HLA-A guide RNA comprises SEQ ID NO: 552. In some embodiments, the HLA-A guide RNA comprises SEQ ID NO: 553. In some embodiments, the HLA-A guide RNA comprises SEQ ID NO: 554. In some embodiments, the HLA-A guide RNA comprises SEQ ID NO: 555. In some embodiments, the HLA-A guide RNA comprises SEQ ID NO: 556. In some embodiments, the HLA-A guide RNA comprises SEQ ID NO: 557. In some embodiments, the HLA-A guide RNA comprises SEQ ID NO: 558. In some embodiments, the HLA-A guide RNA comprises SEQ ID NO: 559. In some embodiments, the HLA-A guide RNA comprises SEQ ID NO: 560. In some embodiments, the HLA-A guide RNA comprises SEQ ID NO: 561. In some embodiments, the HLA-A guide RNA comprises SEQ ID NO: 562. In some embodiments, the HLA-A guide RNA comprises SEQ ID NO: 563. In some embodiments, the HLA-A guide RNA comprises SEQ ID NO: 564. In some embodiments, the HLA-A guide RNA comprises SEQ ID NO: 565. In some embodiments, the HLA-A guide RNA comprises SEQ ID NO: 566. In some embodiments, the HLA-A guide RNA comprises SEQ ID NO: 567. In some embodiments, the HLA-A guide RNA comprises SEQ ID NO: 568. In some embodiments, the HLA-A guide RNA comprises SEQ ID NO: 569. In some embodiments, the HLA-A guide RNA comprises SEQ ID NO: 570. In some embodiments, the HLA-A guide RNA comprises SEQ ID NO: 571. In some embodiments, the HLA-A guide RNA comprises SEQ ID NO: 572. In some embodiments, the HLA-A guide RNA comprises SEQ ID NO: 573. In some embodiments, the HLA-A guide RNA comprises SEQ ID NO: 574. In some embodiments, the HLA-A guide RNA comprises SEQ ID NO: 575. In some embodiments, the HLA-A guide RNA comprises SEQ ID NO: 576. In some embodiments, the HLA-A guide RNA comprises SEQ ID NO: 577. In some embodiments, the HLA-A guide RNA comprises SEQ ID NO: 580. In some embodiments, the HLA-A guide RNA comprises SEQ ID NO: 581. In some embodiments, the HLA-A guide RNA comprises SEQ ID NO: 582. In some embodiments, the HLA-A guide RNA comprises SEQ ID NO: 583. In some embodiments, the HLA-A guide RNA comprises SEQ ID NO: 584. In some embodiments, the HLA-A guide RNA comprises SEQ ID NO: 585. In some embodiments, the HLA-A guide RNA comprises SEQ ID NO: 586. In some embodiments, the HLA-A guide RNA comprises SEQ ID NO: 587. In some embodiments, the HLA-A guide RNA comprises SEQ ID NO: 588. In some embodiments, the HLA-A guide RNA comprises SEQ ID NO: 589. In some embodiments, the HLA-A guide RNA comprises SEQ ID NO: 590.

Additional embodiments of HLA-A guide RNAs are provided herein, including e.g., exemplary modifications to the guide RNA.

### 4. Genetic Modifications to HLA-A

In some embodiments, the methods and compositions disclosed herein genetically modify at least one nucleotide in the HLA-A gene in a cell. Genetic modifications encompass the population of modifications that results from contact with a gene editing system (e.g., the population of edits that result from Cas9 and an HLA-A guide RNA, or the population of edits that result from BC22 and an HLA-A guide RNA). Methods and compositions for genetic modification of the HLA-A gene are provided in PCT/US2021/064930, the entire contents of which is incorporated herein by reference.

The following embodiments are directed to the genetic modification in the HLA-A gene:

In some embodiments, the genetic modification comprises at least one nucleotide within the genomic coordinates chosen from: chr6:29942854-chr6:29942913 and chr6:29943518-chr6: 29943619.

In some embodiments, the genetic modification comprises at least one nucleotide within the genomic coordinates chr6:29942864-chr6: 29942903.

In some embodiments, the genetic modification comprises at least one nucleotide within the genomic coordinates chr6:29943528-chr6:29943609.

In some embodiments, the genetic modification comprises at least one nucleotide within the genomic coordinates chr6:29942864-29942884; chr6:29942868-29942888; chr6:29942876-29942896; chr6:29942877-29942897; and chr6:29942883-29942903.

In some embodiments, the genetic modification comprises at least one nucleotide within the genomic coordinates chr6:29942864-29942884; chr6:29942864-29942884; chr6:29944266-29944290; chr6:29942889-29942913; chr6:29942609-29942633; chr6:29942891-29942915; chr6:29944471-29944495; and chr6:29944470-29944494.

In some embodiments, the genetic modification comprises at least one nucleotide within the genomic coordinates chr6:29942609-29942633; and chr6:29942891-29942915.

In some embodiments, the genetic modification comprises at least one nucleotide within the genomic coordinates chr6:29943528-29943548; chr6:29943529-29943549; chr6:29943530-29943550; chr6:29943537-29943557; chr6:29943549-29943569; and chr6:29943589-29943609.

In some embodiments, the genetic modification comprises at least one nucleotide within the genomic coordinates chr6:29942876-29942897.

In some embodiments, the genetic modification comprises at least one nucleotide within the genomic coordinates chr6:29943528-chr629943550.

In some embodiments, the genetic modification comprises at least one nucleotide within the genomic coordinates chosen from: chr6:29942864-29942884, chr6:29942868-29942888, chr6:29942876-29942896, and chr6:29942877-29942897.

In some embodiments, the genetic modification comprises at least one nucleotide within the genomic coordinates chosen from: chr6:29943528-29943548, chr6:29943529-29943549, and chr6:29943530-29943550.

In some embodiments, the genetic modification comprises at least one nucleotide within the genomic coordinates chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

In some embodiments, the genetic modification comprises an indel, a C to T substitution, or an A to G substitution within the genomic coordinates chosen from; chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

In some embodiments, the genetic modification comprises an indel, a C to T substitution, or an A to G substitution within the genomic coordinates chosen from; chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, chr6:29934330-29934350, chr6:29943115-29943135, chr6:29943135-29943155, chr6:29943140-29943160, chr6:29943590-29943610, chr6:29943824-29943844, chr6:29943858-29943878, chr6:29944478-29944498, and chr6:29944850-29944870.

In some embodiments, the genetic modification comprises an indel, a C to T substitution, or an A to G substitution within the genomic coordinates chosen from; chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

In some embodiments, the genetic modification comprises an indel, a C to T substitution, or an A to G substitution within the genomic coordinates chosen from; chr6:29942864-29942884; chr6:29942868-29942888; chr6:29942876-29942896; chr6:29942877-29942897; chr6:29942883-29942903; chr6:29943528-29943548; chr6:29943529-29943549; chr6:29943530-29943550; chr6:29943537-29943557; chr6:29943549-29943569; and chr6:29943589-29943609.

In some embodiments, the genetic modification comprises an indel, a C to T substitution, or an A to G substitution within the genomic coordinates chosen from; chr6:29942864-29942884; chr6:29942868-29942888; chr6:29942876-29942896; chr6:29942877-29942897; and chr6:29942883-29942903.

In some embodiments, the genetic modification comprises an indel, a C to T substitution, or an A to G substitution within the genomic coordinates chosen from; chr6:29943528-29943548; chr6:29943529-29943549; chr6:29943530-29943550; chr6:29943537-29943557; chr6:29943549-29943569; and chr6:29943589-29943609.

In some embodiments, the genetic modification comprises an indel, a C to T substitution, or an A to G substitution within the genomic coordinates chosen from; chr6:29890117-29890137, chr6:29927058-29927078, chr6:29934330-29934350, chr6:29942541-29942561, chr6:29942542-29942562, chr6:29942543-29942563, chr6:29942543-29942563, chr6:29942550-29942570, chr6:29942864-29942884, chr6:29942868-29942888, chr6:29942876-29942896, chr6:29942876-29942896, chr6:29942877-29942897, chr6:29942883-29942903, chr6:29943062-29943082, chr6:29943063-29943083, chr6:29943092-29943112, chr6:29943115-29943135, chr6:29943118-29943138, chr6:29943119-29943139, chr6:29943120-29943140, chr6:29943126-29943146, chr6:29943128-29943148, chr6:29943129-29943149, chr6:29943134-29943154, chr6:29943134-29943154, chr6:29943135-29943155, chr6:29943136-29943156, chr6:29943140-29943160, chr6:29943142-29943162, chr6:29943143-29943163, chr6:29943188-29943208, chr6:29943528-29943548, chr6:29943529-29943549, chr6:29943530-29943550, chr6:29943536-29943556, chr6:29943537-29943557, chr6:29943538-29943558, chr6:29943549-29943569, chr6:29943556-29943576, chr6:29943589-29943609, chr6:29943590-29943610, chr6:29943590-29943610, chr6:29943599-29943619, chr6:29943600-29943620, chr6:29943601-29943621, chr6:29943602-29943622, chr6:29943603-29943623, chr6:29943774-29943794, chr6:29943779-29943799, chr6:29943780-29943800, chr6:29943822-29943842, chr6:29943824-29943844, chr6:29943857-29943877, chr6:29943858-29943878, chr6:29943859-29943879, chr6:29943860-29943880, chr6:29944026-29944046, chr6:29944077-29944097, chr6:29944078-29944098, chr6:29944458-29944478, chr6:29944478-29944498, chr6:29944597-29944617, chr6:29944642-29944662, chr6:29944643-29944663, chr6:29944772-29944792, chr6:29944782-29944802, chr6:29944850-29944870, chr6:29944907-29944927, chr6:29945024-29945044, chr6:29945097-29945117, chr6:29945104-29945124, chr6:29945105-29945125, chr6:29945116-29945136, chr6:29945118-29945138, chr6:29945119-29945139, chr6:29945124-29945144, chr6:29945176-29945196, chr6:29945177-29945197, chr6:29945177-29945197, chr6:29945180-29945200, chr6:29945187-29945207, chr6:29945188-29945208, chr6:29945228-29945248, chr6:29945230-29945250, chr6:29945231-29945251, chr6:29945232-29945252, chr6:29945308-29945328, chr6:29945361-29945381, chr6:29945362-29945382, and chr6:31382543-31382563.

In some embodiments, the genetic modification comprises an indel, a C to T substitution, or an A to G substitution within the genomic coordinates chosen from; chr6:29942815-29942835, chr6:29942816-29942836, chr6:29942817-29942837, chr6:29942817-29942837, chr6:29942828-29942848, chr6:29942837-29942857, chr6:29942885-29942905, chr6:29942895-29942915, chr6:29942896-29942916, chr6:29942898-29942918, chr6:29942899-29942919, chr6:29942900-29942920, chr6:29942904-29942924, chr6:29942905-29942925, chr6:29942912-29942932, chr6:29942913-29942933, chr6:29943490-29943510, chr6:29943497-29943517, chr6:29943498-29943518, chr6:29943502-29943522, chr6:29943502-29943522, chr6:29943511-29943531, chr6:29943520-29943540, chr6:29943521-29943541, chr6:29943566-29943586, chr6:29943569-29943589, chr6:29943569-29943589, chr6:29943570-29943590, chr6:29943573-29943593, chr6:29943578-29943598, chr6:29943585-29943605, chr6:29943589-29943609, chr6:29943568-29943588, and chr6:29942815-29942835.

In some embodiments, the genetic modification comprises an indel, a C to T substitution, or an A to G substitution within the genomic coordinates chosen from; chr6:29942884-29942904, chr6:29943519-29943539, chr6:29942863-29942883.

In some embodiments, the genetic modification comprises an indel, a C to T substitution, or an A to G substitution within the genomic coordinates chosen from; chr6:29943517-29943537, and chr6:29943523-29943543.

In some embodiments, the genetic modification comprises an indel, a C to T substitution, or an A to G substitution within the genomic coordinates chosen from; chr6:29942845-29942869, chr6:29942852-29942876, chr6:29942865-29942889, chr6:29942891-29942915, chr6:29942895-29942919, chr6:29942903-29942927, chr6:29942904-29942928, chr6:29943518-29943542, chr6:29943525-29943549, chr6:29943535-29943559, chr6:29943538-29943562, chr6:29943539-29943563, chr6:29943547-29943571, chr6:29943547-29943571, chr6:29943548-29943572, chr6:29943555-29943579, chr6:29943556-29943580, chr6:29943557-29943581, chr6:29943558-29943582, chr6:29943559-29943583, chr6:29943563-29943587, chr6:29943564-29943588, chr6:29943565-29943589, chr6:29943568-29943592, chr6:29943571-29943595, chr6:29943572-29943596, chr6:29943595-29943619, chr6:29943596-29943620, chr6:29943600-29943624.

In some embodiments, the genetic modification comprises an indel, a C to T substitution, or an A to G substitution within the genomic coordinates chosen from; chr6:29942885-29942905, chr6:29942895-29942915, chr6:29942896-29942916, chr6:29942898-29942918, chr6:29942899-29942919, chr6:29942900-29942920, chr6:29942904-29942924, chr6:29943511-29943531, chr6:29943520-29943540, chr6:29943521-29943541, chr6:29943529-29943549, chr6:29943566-29943586, chr6:29943568-29943588, chr6:29943569-29943589, chr6:29943569-29943589, chr6:29943570-29943590, chr6:29943573-29943593, chr6:29943578-29943598, chr6:29943585-29943605, and chr6:29943589-29943609.

In some embodiments, the genetic modification comprises an indel, a C to T substitution, or an A to G substitution within the genomic coordinates chosen from; chr6:29942469-29942489, chr6:29943058-29943078, chr6:29943063-29943083, chr6:29943080-29943100, chr6:29943187-29943207, chr6:29943192-29943212, chr6:29943197-29943217, chr6:29943812-29943832, chr6:29944349-29944369, chr6:29944996-29945016, chr6:29945018-29945038, chr6:29945341-29945361, chr6:29945526-29945546.

In some embodiments, the genetic modification comprises an indel, a C to T substitution, or an A to G substitution within the genomic coordinates: chr6:29942876-29942897.

In some embodiments, the genetic modification comprises an indel, a C to T substitution, or an A to G substitution within the genomic coordinates chosen from; chr6:29942864-29942884, chr6:29942868-29942888, chr6:29942876-29942896, and chr6:29942877-29942897.

In some embodiments, the genetic modification comprises an indel, a C to T substitution, or an A to G substitution within the genomic coordinates: chr6:29943528-chr629943550.

In some embodiments, the genetic modification comprises an indel, a C to T substitution, or an A to G substitution within the genomic coordinates chosen from; chr6:29943528-29943548, chr6:29943529-29943549, and chr6:29943530-29943550.

In some embodiments, the modification to HLA-A comprises any one or more of an insertion, deletion, substitution, or deamination of at least one nucleotide in a target sequence. In some embodiments, the modification to HLA-A comprises an insertion of 1, 2, 3, 4 or 5 or more nucleotides in a target sequence. In some embodiments, the modification to HLA-A comprises a deletion of 1, 2, 3, 4 or 5 or more nucleotides in a target sequence. In other embodiments, the modification to HLA-A comprises an insertion of 1, 2, 3, 4, 5, 6, 7, 8, 9, 10, 15, 20 or 25 or more nucleotides in a target sequence. In other embodiments, the modification to HLA-A comprises a deletion of 1, 2, 3, 4, 5, 6, 7, 8, 9, 10, 15, 20 or 25 or more nucleotides in a target sequence. In some embodiments, the modification to HLA-A comprises an indel, which is generally defined in the art as an insertion or deletion of less than 1000 base pairs (bp). In some embodiments, the modification to HLA-A comprises an indel which results in a frameshift mutation in a target sequence. In some embodiments, the modification to HLA-A comprises a substitution of 1, 2, 3, 4, 5, 6, 7, 8, 9, 10, 15, 20 or 25 or more nucleotides in a target sequence. In some embodiments, the modification to HLA-A comprises one or more of an insertion, deletion, or substitution of nucleotides resulting from the incorporation of a template nucleic acid. In some embodiments, the modification to HLA-A comprises an insertion of a donor nucleic acid in a target sequence. In some embodiments, the modification to HLA-A is not transient.

### 5. Efficacy of HLA-A and HLA-B Guide RNAs

The efficacy of an HLA-B guide RNA may be determined by techniques available in the art that assess the editing efficiency of a guide RNA, and the surface expression of HLA-A or HLA-B protein. In some embodiments, the reduction or elimination of surface expression of HLA-A or HLA-B protein may be determined by comparison to an unmodified cell (or “relative to an unmodified cell”). An engineered cell or cell population may also be compared to a population of unmodified cells.

An “unmodified cell” (or “unmodified cells”) refers to a control cell (or cells) of the same type of cell in an experiment or test, wherein the “unmodified” control cell has not been contacted with an HLA-A or HLA-B guide. Therefore, an unmodified cell (or cells) may be a cell that has not been contacted with a guide RNA, or a cell that has been contacted with a guide RNA that does not target HLA-A or HLA-B.

In some embodiments, the efficacy of an HLA-A or HLA-B guide RNA is determined by measuring levels of surface expression of HLA-A or HLA-B protein. In some embodiments, HLA-A or HLA-B protein levels are measured by flow cytometry (e.g., with an antibody against HLA-B7/HLA-B8). Surface expression of HLA-A or HLA-B protein may be measured by flow cytometry as commonly known in the art. One skilled in the art will be familiar with techniques for measuring surface expression of protein such as HLA-A or HLA-B protein, by flow cytometry. An exemplary measurement of levels of surface expression of HLA-A or HLA-B protein by flow cytometry is discussed in Examples 2-3 and 5-8. In some embodiments, the population of cells is enriched (e.g., by FACS or MACS) and is at least 65%, 70%, 80%, 90%, 91%, 92%, 93%, or 94% HLA-A or HLA-B negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% HLA-A or HLA-B negative as measured by flow cytometry relative to a population of unmodified cells. In some embodiments, the population of cells is at least 65% HLA-A or HLA-B negative as measured by flow cytometry relative to a population of unmodified cells. In some embodiments, the population of cells is at least 70% HLA-A or HLA-B negative as measured by flow cytometry relative to a population of unmodified cells. In some embodiments, the population of cells is at least 80% HLA-A or HLA-B negative as measured by flow cytometry relative to a population of unmodified cells. In some embodiments, the population of cells is at least 90% HLA-A or HLA-B negative as measured by flow cytometry relative to a population of unmodified cells. In some embodiments, the population of cells is at least 95% MHC I negative as measured by flow cytometry relative to a population of unmodified cells. In some embodiments, the population of cells is at least 100% HLA-A or HLA-B negative as measured by flow cytometry relative to a population of unmodified cells.

In some embodiments, an effective HLA-A or HLA-B guide RNA may be determined by measuring the response of immune cells in vitro or in vivo (e.g., CD8+ T cells) to the genetically modified target cell. For example, a reduced response from CD8+ T cells is indicative of an effective HLA-A or HLA-B guide RNA. A CD8+ T cell response may be evaluated by an assay that measures CD8+ T cell activation responses, e.g., CD8+ T cell proliferation, expression of activation markers, or cytokine production (IL-2, IFN-γ, TNF-α) (e.g., flow cytometry, ELISA). The CD8+ T cell response may be assessed in vitro or in vivo. In some embodiments, the CD8+ T cell response may be evaluated by co-culturing the genetically modified cell with CD8+ T cells in vitro. In some embodiments, CD8+ T cell activity may be evaluated in an in vivo model, e.g., a rodent model. In an in vivo model, e.g., genetically modified cells may be administered with CD8+ T cell; survival of the genetically modified cells is indicative of the ability to avoid CD8+ T cell lysis. In some embodiments, the methods produce a composition comprising a cell that survives in vivo in the presence of CD8+ T cells for greater than 1, 2, 3, 4, 5, or 6 weeks or more. In some embodiments, the methods produce a composition comprising a cell that survives in vivo in the presence of CD8+ T cells for at least one week to six weeks. In some embodiments, the methods produce a composition comprising a cell that survives in vivo in the presence of CD8+ T cells for at least two to four weeks. In some embodiments, the methods produce a composition comprising a cell that survives in vivo in the presence of CD8+ T cells for at least four to six weeks. In some embodiments, the methods produce a composition comprising a cell that survives in vivo in the presence of CD8+ T cells for more than six weeks.

The efficacy of an HLA-A or HLA-B guide RNA may also be assessed by the survival of the cell post-editing. In some embodiments, the cell survives post editing for at least one week to six weeks. In some embodiments, the cell survives post editing for at least two weeks. In some embodiments, the cell survives post editing for at least three weeks. In some embodiments, the cell survives post editing for at least four weeks. In some embodiments, the cell survives post editing for at least five weeks. In some embodiments, the cell survives post editing for at least six weeks. In some embodiments, the cell survives post editing for at least one week to twelve weeks. The viability of a genetically modified cell may be measured using standard techniques, including e.g., by measures of cell death, by flow cytometry live/dead staining, or cell proliferation.

In some embodiments, the engineered cell is assessed by the persistence of the engineered human cell which has reduced or eliminated surface expression of HLA-B protein and is homozygous for HLA-A and homozygous for HLA-C. In some embodiments, the engineered cell is assessed by the persistence of the engineered human cell which has reduced or eliminated HLA-A and HLA-B expression and is homozygous for HLA-C. As used herein, “persistence” refers to the ability of the engineered cell to exist in an in vitro or in vivo environment with reactive or responding T cells or NK cells present, e.g., the ability to exist in vivo after transfer into a recipient. In some embodiments, the engineered human T cells are protective against NK-mediated rejection. In some embodiments, the ratio of viable engineered cells in vivo in the presence of NK cells relative to viable engineered cells in vivo in the absence of NK cells is at least 0.3:1 or greater, at least 20 days, at least 30 days, at least 40 days, at least 50 days, at least 60 days, at least 70 days, at least 80 days, or at least 90 days after transfer into a recipient, as demonstrated herein. In some embodiments, at least 90 days after transfer into a recipient, the ratio of viable engineered cells in vivo in the presence of NK cells relative to viable engineered cells in vivo in the absence of NK cells is at least 0.4:1 or greater, 0.5:1 or greater, 0.6:1 or greater, 0.7:1 or greater, 0.8:1 or greater, or 0.9:1 or greater, as demonstrated herein. In some embodiments, the engineered human T cells are protective against CD8+ T cell-mediated rejection.

In some embodiments, the engineered cells may be assessed using a mixed lymphocyte reaction (MLR). (See e.g., DeWolf et al., Transplantation 100:1639-1649 (2017). In some embodiments, engineered human cells are mixed with labeled unedited (non-engineered) responding T cells, and the MLR assay measures proliferation of responding T cells activated by allorecognition (i.e., through mismatched HLA molecules on the surface of the engineered human cell).

### D. Methods and Compositions for Reducing or Eliminating MHC Class II, Additional Modifications, and Edited Cells

In some embodiments, multiplex gene editing may be performed in a cell. In some embodiments, the methods comprise reducing or eliminating surface expression of HLA-B protein comprising genetically modifying the HLA-B gene comprising contacting the cell with a composition comprising a HLA-B guide RNA disclosed herein; and optionally an RNA-guided DNA binding agent or a nucleic acid encoding an RNA-guided DNA binding agent, the method further comprising contacting with one or more compositions selected from: (a) a guide RNA that directs an RNA-guided DNA binding agent to the CIITA gene; (b) a guide RNA that directs an RNA-guided DNA binding agent to a locus in the genome of the cell other than HLA-B or CIITA; and (c) a donor nucleic acid for insertion in the genome of the cell.

In some embodiments, multiplex gene editing may be performed in a cell. In some embodiments, the methods comprise reducing or eliminating surface expression of HLA-A and HLA-B protein comprising genetically modifying the HLA-A and HLA-B genes, comprising contacting the cell with a first composition comprising a HLA-A guide RNA disclosed herein; and optionally a first RNA-guided DNA binding agent or a nucleic acid encoding an RNA-guided DNA binding agent; and a second composition comprising a HLA-B guide RNA disclosed herein; and optionally a second RNA-guided DNA binding agent or a nucleic acid encoding an RNA-guided DNA binding agent; the method further comprising contacting with one or more compositions selected from: (a) a guide RNA that directs an RNA-guided DNA binding agent to the CIITA gene; (b) a guide RNA that directs an RNA-guided DNA binding agent to a locus in the genome of the cell other than HLA-A and HLA-B or CIITA; and (c) a donor nucleic acid for insertion in the genome of the cell.

In some embodiments, multiplex gene editing may be performed in a cell. In some embodiments, the methods comprise reducing or eliminating surface expression of HLA-A and HLA-B protein and reducing or eliminating expression of CIITA protein, comprising genetically modifying the HLA-A, HLA-B, and CIITA genes, comprising contacting the cell with a first composition comprising a HLA-A guide RNA disclosed herein; and optionally a first RNA-guided DNA binding agent or a nucleic acid encoding an RNA-guided DNA binding agent; and a second composition comprising a HLA-B guide RNA disclosed herein; and optionally a second RNA-guided DNA binding agent or a nucleic acid encoding an RNA-guided DNA binding agent; and a third composition comprising a CIITA guide RNA disclosed herein; and optionally a third RNA-guided DNA binding agent or a nucleic acid encoding an RNA-guided DNA binding agent; the method further optionally comprising contacting with one or more compositions selected from: (a) a guide RNA that directs an RNA-guided DNA binding agent to a locus in the genome of the cell other than HLA-A, HLA-B, and CIITA, such as TRAC, TRBC1, and/or TRBC2; and (b) a donor nucleic acid for insertion in the genome of the cell.

In some embodiments, one or more compositions for multiplex gene editing in a cell are provided. In some embodiments, the one or more compositions comprise a HLA-A guide RNA disclosed herein, a HLA-B guide RNA disclosed herein, and a CIITA guide RNA disclosed herein; and optionally (a) a guide RNA that directs an RNA-guided DNA binding agent to a locus in the genome of the cell other than HLA-A, HLA-B, and CIITA, such as TRAC, TRBC1, and/or TRBC2; and (b) a donor nucleic acid for insertion in the genome of the cell.

In some embodiments, in any of the methods and compositions disclosed herein, the HLA-A guide RNA is an HLA-A guide RNA that comprises a guide sequence disclosed herein, such as a guide sequence selected from SEQ ID NOs: 301-590. In some embodiments, the HLA-A guide RNA comprises a sequence selected from SEQ ID NOs: 571, 576, 1571, 1576, 2571, 2576, 3111, and 3112. In some embodiments, the HLA-A guide RNA comprises the sequence of SEQ ID NO: 571. In some embodiments, the HLA-A guide RNA comprises the sequence of SEQ ID NO: 576. In some embodiments, the HLA-A guide RNA comprises the sequence of SEQ ID NO: 1571. In some embodiments, the HLA-A guide RNA comprises the sequence of SEQ ID NO: 1576. In some embodiments, the HLA-A guide RNA comprises the sequence of SEQ ID NO: 2571. In some embodiments, the HLA-A guide RNA comprises the sequence of SEQ ID NO: 2576. In some embodiments, the HLA-A guide RNA comprises the sequence of SEQ ID NO: 3111. In some embodiments, the HLA-A guide RNA comprises the sequence of SEQ ID NO: 3112. In some embodiments, in any of the methods and compositions disclosed herein, the HLA-B guide RNA is an HLA-B guide RNA that comprises a guide sequence disclosed herein, such as a guide sequence selected from SEQ ID NOs: 1-91 and 101-185. In some embodiments, the HLA-B guide RNA comprises a sequence selected from SEQ ID NOs: 13, 74, 163-166, 169, 177, 1013, 1074, 1163-1166, 1169, 1177, 2013, 2074, 2163-2166, 2169, 2177, and 2186-2191. In some embodiments, the HLA-B guide RNA comprises the sequence of SEQ ID NO: 13. In some embodiments, the HLA-B guide RNA comprises the sequence of SEQ ID NO: 74. In some embodiments, the HLA-B guide RNA comprises the sequence of SEQ ID NO: 163. In some embodiments, the HLA-B guide RNA comprises the sequence of SEQ ID NO: 164. In some embodiments, the HLA-B guide RNA comprises the sequence of SEQ ID NO: 165. In some embodiments, the HLA-B guide RNA comprises the sequence of SEQ ID NO: 166. In some embodiments, the HLA-B guide RNA comprises the sequence of SEQ ID NO: 169. In some embodiments, the HLA-B guide RNA comprises the sequence of SEQ ID NO: 177. In some embodiments, the HLA-B guide RNA comprises the sequence of SEQ ID NO: 1013. In some embodiments, the HLA-B guide RNA comprises the sequence of SEQ ID NO: 1074. In some embodiments, the HLA-B guide RNA comprises the sequence of SEQ ID NO: 1163. In some embodiments, the HLA-B guide RNA comprises the sequence of SEQ ID NO: 1164. In some embodiments, the HLA-B guide RNA comprises the sequence of SEQ ID NO: 1165. In some embodiments, the HLA-B guide RNA comprises the sequence of SEQ ID NO: 1166. In some embodiments, the HLA-B guide RNA comprises the sequence of SEQ ID NO: 1169. In some embodiments, the HLA-B guide RNA comprises the sequence of SEQ ID NO: 1177. In some embodiments, the HLA-B guide RNA comprises the sequence of SEQ ID NO: 2013. In some embodiments, the HLA-B guide RNA comprises the sequence of SEQ ID NO: 2074. In some embodiments, the HLA-B guide RNA comprises the sequence of SEQ ID NO: 2163. In some embodiments, the HLA-B guide RNA comprises the sequence of SEQ ID NO: 2164. In some embodiments, the HLA-B guide RNA comprises the sequence of SEQ ID NO: 2165. In some embodiments, the HLA-B guide RNA comprises the sequence of SEQ ID NO: 2166. In some embodiments, the HLA-B guide RNA comprises the sequence of SEQ ID NO: 2169. In some embodiments, the HLA-B guide RNA comprises the sequence of SEQ ID NO: 2177. In some embodiments, the HLA-B guide RNA comprises the sequence of SEQ ID NO: 2186. In some embodiments, the HLA-B guide RNA comprises the sequence of SEQ ID NO: 2187. In some embodiments, the HLA-B guide RNA comprises the sequence of SEQ ID NO: 2188. In some embodiments, the HLA-B guide RNA comprises the sequence of SEQ ID NO: 2189. In some embodiments, the HLA-B guide RNA comprises the sequence of SEQ ID NO: 2190. In some embodiments, the HLA-B guide RNA comprises the sequence of SEQ ID NO: 2191. In some embodiments, in any of the methods and compositions disclosed herein, the CIITA guide RNA is a CIITA guide RNA that comprises a guide sequence disclosed herein, such as SEQ ID NO: 608 or 609. In some embodiments, the CIITA guide RNA comprises a sequence selected from SEQ ID NOs: 608, 609, 1608, 1609, 2608, 2609, 3116, and 3117. In some embodiments, the CIITA guide RNA comprises the sequence of SEQ ID NO: 608. In some embodiments, the CIITA guide RNA comprises the sequence of SEQ ID NO: 609. In some embodiments, the CIITA guide RNA comprises the sequence of SEQ ID NO: 1608. In some embodiments, the CIITA guide RNA comprises the sequence of SEQ ID NO: 1609. In some embodiments, the CIITA guide RNA comprises the sequence of SEQ ID NO: 2608. In some embodiments, the CIITA guide RNA comprises the sequence of SEQ ID NO: 2609. In some embodiments, the CIITA guide RNA comprises the sequence of SEQ ID NO: 3116. In some embodiments, the CIITA guide RNA comprises the sequence of SEQ ID NO: 3117. In some embodiments, in any of the methods and compositions disclosed herein, the guide RNA that directs an RNA-guided DNA binding agent to a locus in the genome of the cell other than HLA-A, HLA-B, and CIITA comprises a TRAC guide RNA and/or a TRBC guide RNA. In some embodiments, the TRAC guide RNA comprises a sequence selected from SEQ ID NOs: 605, 606, 613, 1605, 1606, 1613, 2605, 2606, 2613, 3113, and 3114. In some embodiments, the TRAC guide RNA comprises the sequence of SEQ ID NO: 605. In some embodiments, the TRAC guide RNA comprises the sequence of SEQ ID NO: 606. In some embodiments, the TRAC guide RNA comprises the sequence of SEQ ID NO: 613. In some embodiments, the TRAC guide RNA comprises the sequence of SEQ ID NO: 1605. In some embodiments, the TRAC guide RNA comprises the sequence of SEQ ID NO: 1606. In some embodiments, the TRAC guide RNA comprises the sequence of SEQ ID NO: 1613. In some embodiments, the TRAC guide RNA comprises the sequence of SEQ ID NO: 2605. In some embodiments, the TRAC guide RNA comprises the sequence of SEQ ID NO: 2606. In some embodiments, the TRAC guide RNA comprises the sequence of SEQ ID NO: 2613. In some embodiments, the TRAC guide RNA comprises the sequence of SEQ ID NO: 3113. In some embodiments, the TRAC guide RNA comprises the sequence of SEQ ID NO: 3114. In some embodiments, the TRBC guide RNA comprises a sequence selected from SEQ ID NOs: 607, 1607, 2607, and 3115. In some embodiments, the TRBC guide RNA comprises the sequence of SEQ ID NO: 607. In some embodiments, the TRBC guide RNA comprises the sequence of SEQ ID NO: 1607. In some embodiments, the TRBC guide RNA comprises the sequence of SEQ ID NO: 2607. In some embodiments, the TRBC guide RNA comprises the sequence of SEQ ID NO: 3115.

In some embodiments, edited cells obtained by the multiplex gene editing methods or compositions are provided. In some embodiments, the edited cells comprise a genetic modification in the HLA-A gene, a genetic modification in the HLA-B gene, and a genetic modification in the CIITA gene; and optionally a genetic modification in a gene other than HLA-A, HLA-B, and CIITA, such as TRAC, TRBC1, and/or TRBC2. In some embodiments, the genetic modification in the HLA-A gene comprises at least one nucleotide within the genomic coordinates chosen from: chr6:29942891-29942915; or chr6:29942609-29942633. In some embodiments, the genetic modification in the HLA-A gene comprises at least one nucleotide within the genomic coordinates chr6:29942891-29942915. In some embodiments, the genetic modification in the HLA-A gene comprises at least one nucleotide within the genomic coordinates chr6:29942609-29942633. In some embodiments, the genetic modification in the HLA-B gene comprises at least one nucleotide within the genomic coordinates chosen from: (a) chr6:31355348-31355368; or chr6:31355347-31355367; or (b) chr6:31355221-31355245; chr6:31355222-31355246; chr6:31355205-31355229; chr6:31355446-31355470; chr6:31356425-31356449; or chr6:31355441-31355465. In some embodiments, the genetic modification in the HLA-B gene comprises at least one nucleotide within the genomic coordinates chr6:31355348-31355368. In some embodiments, the genetic modification in the HLA-B gene comprises at least one nucleotide within the genomic coordinates chr6:31355347-31355367. In some embodiments, the genetic modification in the HLA-B gene comprises at least one nucleotide within the genomic coordinates chr6:31355221-31355245. In some embodiments, the genetic modification in the HLA-B gene comprises at least one nucleotide within the genomic coordinates chr6:31355222-31355246. In some embodiments, the genetic modification in the HLA-B gene comprises at least one nucleotide within the genomic coordinates chr6:31355205-31355229. In some embodiments, the genetic modification in the HLA-B gene comprises at least one nucleotide within the genomic coordinates chr6:31355446-31355470. In some embodiments, the genetic modification in the HLA-B gene comprises at least one nucleotide within the genomic coordinates chr6:31356425-31356449. In some embodiments, the genetic modification in the HLA-B gene comprises at least one nucleotide within the genomic coordinates chr6:31355441-31355465. In some embodiments, the genetic modification in the CIITA gene comprises at least one nucleotide within the genomic coordinates chosen from: chr16:10907504-10907528 or chr16:10906643-10906667. In some embodiments, the genetic modification in the CIITA gene comprises at least one nucleotide within the genomic coordinates chr16:10907504-10907528. In some embodiments, the genetic modification in the CIITA gene comprises at least one nucleotide within the genomic coordinates chr16:10906643-10906667. In some embodiments, the genetic modification in the TRAC gene comprises at least one nucleotide within the genomic coordinates chosen from: chr14:22550574-22550598 or chr14:22550544-22550568. In some embodiments, the genetic modification in the TRAC gene comprises at least one nucleotide within the genomic coordinates chr14:22550574-22550598. In some embodiments, the genetic modification in the TRAC gene comprises at least one nucleotide within the genomic coordinates chr14:22550544-22550568. In some embodiments, the genetic modification in the TRBC gene comprises at least one nucleotide within the genomic coordinates chr7:142792690-142792714.

### 1. MHC Class H Knock Out

In some embodiments, methods for reducing or eliminating surface expression of HLA-B by genetically modifying HLA-B as disclosed herein are provided, wherein the methods and compositions further provide for reducing or eliminating surface expression of MHC class II protein relative to an unmodified cell. In some embodiments, MHC class II protein expression is reduced or eliminated by contacting the cell with a CIITA guide RNA. In some embodiments, the cell is an allogeneic cell. In some embodiments, the cell is homozygous for HLA-A and homozygous for HLA-C.

In some embodiments, methods for reducing or eliminating surface expression of HLA-A and HLA-B protein by genetically modifying HLA-A and HLA-B genes as disclosed herein are provided, wherein the methods and compositions further provide for reducing or eliminating surface expression of MHC class II protein relative to an unmodified cell. In some embodiments, MHC class II protein expression is reduced or eliminated by contacting the cell with a CIITA guide RNA. In some embodiments, the cell is an allogeneic cell. In some embodiments, the cell is homozygous for HLA-C.

In some embodiments, methods are provided for reducing surface expression of MHC class II protein on the engineered human cell. MHC class II expression is impacted by a variety of proteins. (See e.g., Crivello et al., Journal Immunology 202:1895-1903 (2019).) For example, the CIITA protein functions as a transcriptional activator (activating the MHC class II promoter) and is essential for MHC class II protein expression. In some embodiments, MHC class II protein expression is reduced or eliminated by genetically modifying a gene selected from: CIITA, HLA-DR, HLA-DQ, HLA-DP, RFX5, RFXB/ANK, RFXAP, CREB, NF-YA, NF-YB, and NF-YC. In some embodiments, MHC class II protein expression is reduced or eliminated by genetically modifying the CIITA gene. In some embodiments, MHC class II protein expression is reduced or eliminated by genetically modifying the HLA-DR gene. In some embodiments, MHC class II protein expression is reduced or eliminated by genetically modifying the HLA-DQ gene. In some embodiments, MHC class II protein expression is reduced or eliminated by genetically modifying the HLA-DP gene. In some embodiments, MHC class II protein expression is reduced or eliminated by genetically modifying the RFX5 gene. In some embodiments, MHC class II protein expression is reduced or eliminated by genetically modifying the RFXB/ANK gene. In some embodiments, MHC class II protein expression is reduced or eliminated by genetically modifying the RFXAP gene. In some embodiments, MHC class II protein expression is reduced or eliminated by genetically modifying the CREB gene. In some embodiments, MHC class II protein expression is reduced or eliminated by genetically modifying the NK-YA gene. In some embodiments, MHC class II protein expression is reduced or eliminated by genetically modifying the NK-YB gene. In some embodiments, MHC class II protein expression is reduced or eliminated by genetically modifying the NK-YC gene.

In some embodiments, methods are provided for making an engineered human cell which has reduced or eliminated expression of HLA-B protein relative to an unmodified cell, wherein the cell is homozygous for HLA-A and homozygous for HLA-C, further comprising reducing or eliminating the surface expression of MHC class II protein in the cell relative to an unmodified cell. In some embodiments, the methods comprise contacting the cell with a CIITA guide RNA.

In some embodiments, methods are provided for making an engineered human cell which has reduced or eliminated expression of HLA-A and HLA-B protein relative to an unmodified cell, wherein the cell homozygous for HLA-C, further comprising reducing or eliminating the surface expression of MHC class II protein in the cell relative to an unmodified cell. In some embodiments, the methods comprise contacting the cell with a CIITA guide RNA.

In some embodiments, the efficacy of a CIITA guide RNA is determined by measuring levels of CIITA protein in a cell. The levels of CIITA protein may be detected by, e.g., cell lysate and western blot with an anti-CIITA antibody. In some embodiments, the efficacy of a CIITA guide RNA is determined by measuring levels of CIITA protein in the cell nucleus. In some embodiments, the efficacy of a CIITA guide RNA is determined by measuring levels of CIITA mRNA in a cell. The levels of CIITA mRNA may be detected by e.g., RT-PCR. In some embodiments, a decrease in the levels CIITA protein or CIITA mRNA in the target cell as compared to an unmodified cell is indicative of an effective CIITA guide RNA.

In some embodiments, the efficacy of a CIITA guide RNA is determined by measuring the reduction or elimination of MHC class II protein expression by the target cells. The CIITA protein functions as a transactivator, activating the MHC class II promoter, and is essential for the expression of MHC class II protein. In some embodiments, MHC class II protein expression may be detected on the surface of the target cells. In some embodiments, MHC class II protein expression is measured by flow cytometry. In some embodiments, an antibody against MHC class II protein (e.g., anti-HLA-DR, -DQ, -DP) may be used to detect MHC class II protein expression e.g., by flow cytometry. In some embodiments, a reduction or elimination in MHC class II protein on the surface of a cell (or population of cells) as compared to an unmodified cell (or population of unmodified cells) is indicative of an effective CIITA guide RNA. In some embodiments, a cell (or population of cells) that has been contacted with a particular CIITA guide RNA and RNA-guided DNA binding agent that is negative for MHC class II protein by flow cytometry is indicative of an effective CIITA guide RNA.

In some embodiments, the MHC class II protein expression is reduced or eliminated in a population of cells using the methods and compositions disclosed herein. In some embodiments, the population of cells is enriched (e.g., by FACS or MACS) and is at least 65%, 70%, 80%, 90%, 91%, 92%, 93%, or 94% MHC class II negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% MHC class II negative as measured by flow cytometry relative to a population of unmodified cells.

In some embodiments, the population of cells is at least 65% MHC II negative as measured by flow cytometry relative to a population of unmodified cells. In some embodiments, the population of cells is at least 70% MHC class II negative as measured by flow cytometry relative to a population of unmodified cells. In some embodiments, the population of cells is at least 80% MHC II negative as measured by flow cytometry relative to a population of unmodified cells. In some embodiments, the population of cells is at least 90% MHC class II negative as measured by flow cytometry relative to a population of unmodified cells. In some embodiments, the population of cells is at least 91% MHC class II negative as measured by flow cytometry relative to a population of unmodified cells. In some embodiments, the population of cells is at least 92% MHC II negative as measured by flow cytometry relative to a population of unmodified cells. In some embodiments, the population of cells is at least 93% MHC class II negative as measured by flow cytometry relative to a population of unmodified cells. In some embodiments, the population of cells is at least 94% MHC class II negative as measured by flow cytometry relative to a population of unmodified cells.

In some embodiments, the population of cells elicits a reduced response from immune cells in vitro or in vivo (e.g., CD4+ T cells). A CD4+ T cell response may be evaluated by an assay that measures the activation response of CD4+ T cells e.g., CD4+ T cell proliferation, expression of activation markers, or cytokine production (IL-2, IL-12, IFN-γ) (e.g., flow cytometry, ELISA). The response of CD4+ T cells may be evaluated in in vitro cell culture assays in which the genetically modified cell is co-cultured with cells comprising CD4+ T cells. For example, the engineered cell may be co-cultured e.g., with PBMCs, purified CD3+ T cells comprising CD4+ T cells, purified CD4+ T cells, or a CD4+ T cell line. The CD4+ T cell response elicited from the engineered cell may be compared to the response elicited from an unmodified cell.

In some embodiments, an engineered human cell is provided wherein the cell has reduced or eliminated surface expression of HLA-B and MHC class II protein wherein the cell comprises a genetic modification in the HLA-B gene, wherein the cell is homozygous for HLA-A and HLA-C, and wherein the cell comprises a modification in the CIITA gene. In some embodiments, the engineered cell elicits a reduced response from CD4+ T cells and elicits a reduced response from CD8+ T cells.

In some embodiments, an engineered human cell is provided wherein the cell has reduced or eliminated surface expression of HLA-A, HLA-B, and MHC class II protein, wherein the cell comprises a genetic modification in the HLA-A and HLA-B genes, wherein the cell is homozygous for HLA-C, and wherein the cell comprises a modification in the CIITA gene. In some embodiments, the engineered cell elicits a reduced response from CD4+ T cells and elicits a reduced response from CD8+ T cells.

### 2. Exogenous Nucleic Acids Knock in

In some embodiments, the present disclosure provides methods and compositions for reducing or eliminating surface expression of HLA-B protein by genetically modifying HLA-B as disclosed herein, wherein the methods and compositions further provide for expression of a protein encoded by an exogenous nucleic acid (e.g., an antibody, chimeric antigen receptor (CAR), T cell receptor (TCR), cytokine or cytokine receptor, chemokine or chemokine receptor, enzyme, fusion protein, or other type of cell-surface bound or soluble polypeptide). In some embodiments, the exogenous nucleic acid encodes a protein that is expressed on the cell surface. For example, in some embodiments, the exogenous nucleic acid encodes a targeting receptor expressed on the cell surface (described further herein). In some embodiments, the genetically modified cell may function as a “cell factory” for the expression of a secreted polypeptide encoded by an exogenous nucleic acid, including e.g., as a source for continuous production of a polypeptide in vivo (as described further herein). In some embodiments, the cell is an allogeneic cell. In some embodiments, the cell is homozygous for HLA-A and homozygous for HLA-C.

In some embodiments, the present disclosure provides methods and compositions for reducing or eliminating surface expression of HLA-A and HLA-B protein by genetically modifying HLA-A and HLA-B as disclosed herein, wherein the methods and compositions further provide for expression of a protein encoded by an exogenous nucleic acid (e.g., an antibody, chimeric antigen receptor (CAR), T cell receptor (TCR), cytokine or cytokine receptor, chemokine or chemokine receptor, enzyme, fusion protein, or other type of cell-surface bound or soluble polypeptide). In some embodiments, the exogenous nucleic acid encodes a protein that is expressed on the cell surface. For example, in some embodiments, the exogenous nucleic acid encodes a targeting receptor expressed on the cell surface (described further herein). In some embodiments, the targeting receptor is a CAR. In some embodiments, the targeting receptor is a universal CAR. In some embodiments, the targeting receptor is an anti-CD30 CAR. In some embodiments, the anti-CD30 CAR is any one of the anti-CD30 CARs disclosed in International Application No. PCT/US2023/018946, the content of which is incorporated herein by reference. In some embodiments, the genetically modified cell may function as a “cell factory” for the expression of a secreted polypeptide encoded by an exogenous nucleic acid, including e.g., as a source for continuous production of a polypeptide in vivo (as described further herein). In some embodiments, the cell is an allogeneic cell. In some embodiments, the cell is homozygous for HLA-C.

In some embodiments, the methods comprise reducing surface expression of HLA-B protein comprising genetically modifying the HLA-B gene comprising contacting the cell with a composition comprising an HLA-B guide RNA disclosed herein, the method further comprising contacting the cell with an exogenous nucleic acid.

In some embodiments, the methods comprise reducing surface expression of HLA-A and HLA-B protein comprising genetically modifying the HLA-A and HLA-B genes comprising contacting the cell with a first composition comprising an HLA-A guide RNA disclosed herein and a second composition comprising an HLA-B guide RNA disclosed herein, the method further comprising contacting the cell with an exogenous nucleic acid.

In some embodiments, the methods comprise reducing or eliminating surface expression of HLA-B protein, comprising genetically modifying the cell with one or more compositions comprising an HLA-B guide RNA as disclosed herein, an exogenous nucleic acid encoding a polypeptide (e.g., a targeting receptor), and an RNA-guided DNA binding agent or a nucleic acid encoding an RNA-guided DNA binding agent.

In some embodiments, the methods comprise reducing or eliminating surface expression of HLA-A and HLA-B protein, comprising genetically modifying the cell with one or more compositions comprising a first composition comprising an HLA-A guide RNA as disclosed herein, a second composition comprising an HLA-B guide RNA as disclosed herein, an exogenous nucleic acid encoding a polypeptide (e.g., a targeting receptor), and one or more RNA-guided DNA binding agent or a nucleic acid encoding an RNA-guided DNA binding agent.

In some embodiments, the methods comprise reducing or eliminating surface expression of HLA-B protein and MHC class II protein, comprising genetically modifying the cell with one or more compositions comprising a HLA-B guide RNA as disclosed herein, a CIITA guide RNA, an exogenous nucleic acid encoding a polypeptide (e.g., a targeting receptor), and an RNA-guided DNA binding agent or a nucleic acid encoding an RNA-guided DNA binding agent.

In some embodiments, the methods comprise reducing or eliminating surface expression of HLA-A and HLA-B protein and MHC class II protein, comprising genetically modifying the cell with one or more compositions comprising a first composition comprising an HLA-A guide RNA as disclosed herein, a second composition comprising an HLA-B guide RNA as disclosed herein, a CIITA guide RNA, an exogenous nucleic acid encoding a polypeptide (e.g., a targeting receptor), and one or more RNA-guided DNA binding agent or a nucleic acid encoding an RNA-guided DNA binding agent.

In some embodiments, the exogenous nucleic acid encodes a polypeptide that is expressed on the surface of the cell. In some embodiments, the exogenous nucleic acid encodes a soluble polypeptide. As used herein, “soluble” polypeptide refers to a polypeptide that is secreted by the cell. In some embodiments, the soluble polypeptide is a therapeutic polypeptide. In some embodiments, the soluble polypeptide is an antibody. In some embodiments, the soluble polypeptide is an enzyme. In some embodiments, the soluble polypeptide is a cytokine. In some embodiments, the soluble polypeptide is a chemokine. In some embodiments, the soluble polypeptide is a fusion protein.

In some embodiments, the exogenous nucleic acid encodes an antibody. In some embodiments, the exogenous nucleic acid encodes an antibody fragment (e.g., Fab, Fab2). In some embodiments, the exogenous nucleic acid encodes is a full-length antibody. In some embodiments, the exogenous nucleic acid encodes is a single-chain antibody (e.g., scFv). In some embodiments, the antibody is an IgG, IgM, IgD, IgA, or IgE. In some embodiments, the antibody is an IgG antibody. In some embodiments, the antibody is an IgG1 antibody. In some embodiments, the antibody is an IgG4 antibody. In some embodiments, the heavy chain constant region contains mutations known to reduce effector functions. In some embodiments, the heavy chain constant region contains mutations known to enhance effector functions. In some embodiments, the antibody is a bispecific antibody. In some embodiments, the antibody is a single-domain antibody (e.g., VH domain-only antibody).

In some embodiments, the exogenous nucleic acid encodes a neutralizing antibody. A neutralizing antibody neutralizes the activity of its target antigen. In some embodiments, the antibody is a neutralizing antibody against a virus antigen. In some embodiments, the antibody neutralizes a target viral antigen, blocking the ability of the virus to infect a cell. In some embodiments, a cell-based neutralization assay may be used to measure the neutralizing activity of an antibody. The particular cells and readout will depend on the target antigen of the neutralizing antibody. The half maximal effective concentration (EC₅₀) of the antibody can be measured in a cell-based neutralization assay, wherein a lower EC₅₀ is indicative of more potent neutralizing antibody.

In some embodiments, the exogenous nucleic acid encodes an antibody that binds to an antigen associated with a disease or disorder (see e.g., diseases and disorders described in Section IV).

In some embodiments, the exogenous nucleic acid encodes a polypeptide that is expressed on the surface of the cell (i.e., a cell-surface bound protein). In some embodiments, the exogenous nucleic acid encodes a targeting receptor. A “targeting receptor” is a receptor present on the surface of a cell, e.g., a T cell, to permit binding of the cell to a target site, e.g., a specific cell or tissue in an organism. In some embodiments, the targeting receptor is a CAR. In some embodiments, the targeting receptor is a universal CAR (UniCAR). In some embodiments, the targeting receptor is a proliferation-inducing ligand (APRIL). In some embodiments, the targeting receptor is a TCR. In some embodiments, the targeting receptor is a TRuC. In some embodiments, the targeting receptor is a B cell receptor (BCR) (e.g., expressed on a B cell). In some embodiments, the targeting receptor is chemokine receptor. In some embodiments, the targeting receptor is a cytokine receptor.

In some embodiments, targeting receptors include a chimeric antigen receptor (CAR), a T-cell receptor (TCR), and a receptor for a cell surface molecule operably linked through at least a transmembrane domain in an internal signaling domain capable of activating a T cell upon binding of the extracellular receptor portion. In some embodiments, a CAR refers to an extracellular antigen recognition domain, e.g., an scFv, VHH, nanobody; operably linked to an intracellular signaling domain, which activates the T cell when an antigen is bound. CARs are composed of four regions: an antigen recognition domain, an extracellular hinge region, a transmembrane domain, and an intracellular T-cell signaling domain. Such receptors are well known in the art (see, e.g., WO2020092057, WO2019191114, WO2019147805, WO2018208837). A universal CAR (UniCAR) for recognizing various antigens (see, e.g., EP 2 990 416 A1) and a reversed universal CAR (RevCAR) that promotes binding of an immune cell to a target cell through an adaptor molecule (see, e.g., WO2019238722) are also contemplated. CARs can be targeted to any antigen to which an antibody can be developed and are typically directed to molecules displayed on the surface of a cell or tissue to be targeted. In some embodiments, the targeting receptor comprises an antigen recognition domain (e.g., a cancer antigen recognition domain and a subunit of a TCR (e.g., a TRuC). (See Baeuerle et al. Nature Communications 2087 (2019).)

In some embodiments, the exogenous nucleic acid encodes a TCR. In some embodiments, the exogenous nucleic acid encodes a genetically modified TCR. In some embodiments, the exogenous nucleic acid encodes is a genetically modified TCR with specificity for a polypeptide expressed by cancer cells. In some embodiments, the exogenous nucleic acid encodes a targeting receptor specific for Wilms' tumor gene (WT1) antigen. In some embodiments, the exogenous nucleic acid encodes the WT1-specific TCR (see e.g., WO2020/081613A1).

In some embodiments, an exogenous nucleic acid is inserted into the genome of the target cell. In some embodiments, the exogenous nucleic acid is integrated into the genome of the target cell. In some embodiments, the exogenous nucleic acid is integrated into the genome of the target cell by homologous recombination (HR). In some embodiments, the exogenous nucleic acid is integrated into the genome of the target cell by blunt end insertion. In some embodiments, the exogenous nucleic acid is integrated into the genome of the target cell by non-homologous end joining. In some embodiments, the exogenous nucleic acid is integrated into a safe harbor locus in the genome of the cell. In some embodiments, the exogenous nucleic acid is integrated into one of the TRAC locus, B2M locus, AAVS1 locus, or CIITA locus. In some embodiments, the lipid nucleic acid assembly composition is a lipid nanoparticle (LNP).

In some embodiments, the methods produce a composition comprising an engineered cell having reduced or eliminated surface expression of HLA-B protein and comprising an exogenous nucleic acid. In some embodiments, the methods produce a composition comprising an engineered cell having reduced or eliminated surface expression of HLA-B protein and that secretes or expresses a polypeptide encoded by an exogenous nucleic acid integrated into the genome of the cell. In some embodiments, the methods produce a composition comprising an engineered cell having reduced or eliminated surface expression of HLA-B protein, or reduced or eliminated HLA-B levels in the cell nucleus, and having reduced or eliminated surface expression of MHC class II protein expression, and secreting or expressing a polypeptide encoded by an exogenous nucleic acid integrated into the genome of the cell. In some embodiments, the engineered cell elicits a reduced response from CD4+ T cells, or CD8+ T cells.

In some embodiments, the methods produce a composition comprising an engineered cell having reduced or eliminated surface expression of HLA-A and HLA-B protein and comprising an exogenous nucleic acid. In some embodiments, the methods produce a composition comprising an engineered cell having reduced or eliminated surface expression of HLA-A and HLA-B protein and that secretes or expresses a polypeptide encoded by an exogenous nucleic acid integrated into the genome of the cell. In some embodiments, the methods produce a composition comprising an engineered cell having reduced or eliminated surface expression of HLA-A and HLA-B protein, or reduced or eliminated HLA-A and HLA-B levels in the cell nucleus, and having reduced surface expression of MHC class II protein, and secreting or expressing a polypeptide encoded by an exogenous nucleic acid integrated into the genome of the cell. In some embodiments, the engineered cell elicits a reduced response from CD4+ T cells, or CD8+ T cells.

In some embodiments, an allogeneic cell is provided wherein the cell has reduced or eliminated surface expression of MHC class II and HLA-B protein, wherein the cell comprises a modification in the HLA-B gene as disclosed herein, wherein the cell comprises a modification in the CIITA gene, and wherein the cell further comprises an exogenous nucleic acid encoding a polypeptide (e.g., a targeting receptor).

In some embodiments, an allogeneic cell is provided wherein the cell has reduced or eliminated surface expression of MHC class II, HLA-A, and HLA-B protein, wherein the cell comprises a modification in the HLA-A and HLA-B gene as disclosed herein, wherein the cell comprises a modification in the CIITA gene, and wherein the cell further comprises an exogenous nucleic acid encoding a polypeptide (e.g., a targeting receptor).

In some embodiments, the present disclosure provides methods for reducing or eliminating surface expression of HLA-B protein by genetically modifying HLA-B as disclosed herein, wherein the methods further provide for reducing expression of one or more additional target genes (e.g., TRAC, TRBC). In some embodiments, the additional genetic modifications provide further advantages for use of the genetically modified cells for adoptive cell transfer applications. In some embodiments, the cell is an allogeneic cell. In some embodiments, the cell is homozygous for HLA-A and homozygous for HLA-C.

In some embodiments, the present disclosure provides methods for reducing or eliminating surface expression of HLA-A and HLA-B protein by genetically modifying HLA-A and HLA-B as disclosed herein, wherein the methods further provide for reducing expression of one or more additional target genes (e.g., TRAC, TRBC). In some embodiments, the additional genetic modifications provide further advantages for use of the genetically modified cells for adoptive cell transfer applications. In some embodiments, the cell is an allogeneic cell. In some embodiments, the cell is homozygous for HLA-C.

In some embodiments, the methods comprise reducing or eliminating surface expression of HLA-B protein, comprising genetically modifying the cell with one or more compositions comprising a HLA-B guide RNA as disclosed herein, a CIITA guide RNA, an exogenous nucleic acid encoding polypeptide (e.g., a targeting receptor), a guide RNA that directs an RNA-guided DNA binding agent to a target sequence located in an another gene, thereby reducing or eliminating expression of the other gene, and an RNA-guided DNA binding agent or a nucleic acid encoding an RNA-guided DNA binding agent. In some embodiments, the additional target gene is TRAC. In some embodiments, the additional target gene is TRBC.

In some embodiments, the methods comprise reducing or eliminating surface expression of HLA-A and HLA-B protein, comprising genetically modifying the cell with one or more compositions comprising a HLA-B guide RNA as disclosed herein, a CIITA guide RNA, an exogenous nucleic acid encoding polypeptide (e.g., a targeting receptor), a guide RNA that directs an RNA-guided DNA binding agent to a target sequence located in an another gene, thereby reducing or eliminating expression of the other gene, and an RNA-guided DNA binding agent or a nucleic acid encoding an RNA-guided DNA binding agent. In some embodiments, the additional target gene is TRAC. In some embodiments, the additional target gene is TRBC.

In some embodiments, the method disclosed herein further comprises contacting the cell with a DNA-dependent protein kinase inhibitor (DNAPK), optionally wherein the DNAPKi is Compound 1 or “DNAPKI Compound 1”: 9-(4,4-difluorocyclohexyl)-7-methyl-2-((7-methyl-[1,2,4]triazolo[1,5-a]pyridin-6-yl)amino)-7,9-dihydro-8H-purin-8-one, also depicted as:

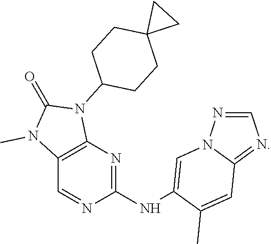 [SMILES: Cc1cc2ncnn2cc1Nc1ncc2c(n1)n(C1CCC3(CC1)CC3)c(=O)n2C]

### E. Exemplary Cell Types

In some embodiments, methods and compositions disclosed herein genetically modify a human cell. In some embodiments, the cell is an allogeneic cell. In some embodiments the genetically modified cell is referred to as an engineered cell. An engineered cell refers to a cell (or progeny of a cell) comprising an engineered genetic modification, e.g. that has been contacted with a gene editing system and genetically modified by the gene editing system. The terms “engineered cell” and “genetically modified cell” are used interchangeably throughout. The engineered human cell may be any of the exemplary cell types disclosed herein. Further, because MHC class I molecules are expressed on all nucleated cells, the engineered human cell may be any nucleated cell.

In some embodiments, when the cell is homozygous for HLA-A, the HLA-A allele is selected from any one of the following HLA-A alleles: HLA-A*02:01; HLA-A*01:01; HLA-A*03:01; HLA-A*11:01; HLA-A*26:01; HLA-A*68:01; HLA-A*29:02; HLA-A*31:01; HLA-A*32:01; HLA-A*30:02; HLA-A*25:01; HLA-A*33:01; HLA-A*02:02; HLA-A*74:01; HLA-A*02:02; HLA-A*29:01; HLA-A*02:03; HLA-A*02:05; HLA-A*24:07; HLA-A*11:02; HLA-A*36:01; HLA-A*02:22; HLA-A*34:02; HLA-A*01:03; HLA-A*24:02; HLA-A*02:07; HLA-A*23:01; HLA-A*30:01; HLA-A*33:03; HLA-A*02:06; HLA-A*34:02; and HLA-A*68:02.

In some embodiments, when the cell is homozygous for HLA-C, the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*08:01; HLA-C*03:02; HLA-C*16:01; HLA-C*15:02; HLA-C*03:04; HLA-C*12:03; HLA-C*02:10; HLA-C*05:01; HLA-C*12:02; HLA-C*14:02; HLA-C*04:01; HLA-C*03:03; HLA-C*07:04; HLA-C*17:01; HLA-C*01:02; and HLA-C*02:02.

In some embodiments, when the cell is homozygous for HLA-C, the HLA-C allele is HLA-C*03:04. In some embodiments, when the cell is homozygous for HLA-C, the HLA-C allele is HLA-C*06:02. In some embodiments, when the cell is homozygous for HLA-C, the HLA-C allele is HLA-C*01:02. In some embodiments, when the cell is homozygous for HLA-C, the HLA-C allele is HLA-C*08:01. In some embodiments, when the cell is homozygous for HLA-C, the HLA-C allele is HLA-C*03:02.

In some embodiments, when the cell is homozygous for HLA-A and homozygous for HLA-C, and the HLA-A allele is selected from any one of the following HLA-A alleles: HLA-A*02:01; HLA-A*01:01; HLA-A*03:01; HLA-A*11:01; HLA-A*26:01; HLA-A*68:01; HLA-A*29:02; HLA-A*31:01; HLA-A*32:01; HLA-A*30:02; HLA-A*25:01; HLA-A*33:01; HLA-A*02:02; HLA-A*74:01; HLA-A*02:02; HLA-A*29:01; HLA-A*02:03; HLA-A*02:05; HLA-A*24:07; HLA-A*11:02; HLA-A*36:01; HLA-A*02:22; HLA-A*34:02; HLA-A*01:03; HLA-A*24:02; HLA-A*02:07; HLA-A*23:01; HLA-A*30:01; HLA-A*33:03; HLA-A*02:06; HLA-A*34:02; and HLA-A*68:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*08:01; HLA-C*03:02; HLA-C*16:01; HLA-C*15:02; HLA-C*03:04; HLA-C*12:03; HLA-C*02:10; HLA-C*05:01; HLA-C*12:02; HLA-C*14:02; HLA-C*04:01; HLA-C*03:03; HLA-C*07:04; HLA-C*17:01; HLA-C*01:02; and HLA-C*02:02.

In some embodiments, when the cell is homozygous for HLA-A and homozygous for HLA-C, and the HLA-A and HLA-C alleles are selected from any one of the following HLA-A and HLA-C alleles: HLA-A*01:01 and HLA-C*07:01; HLA-A*02:01 and HLA-C*07:02; HLA-A*02:01 and HLA-C*05:01; HLA-A*03:01 and HLA-C*07:02; HLA-A*02:01 and HLA-C*04:01; HLA-A*02:01 and HLA-C*03:04; HLA-A*01:01 and HLA-C*06:02; HLA-A*03:01 and HLA-C*04:01; HLA-A*02:01 and HLA-C*07:01; HLA-A*24:02 and HLA-C*04:01; HLA-A*29:02 and HLA-C*16:01; HLA-A*02:01 and HLA-C*06:02; HLA-A*24:02 and HLA-C*07:02; HLA-A*26:01 and HLA-C*12:03; HLA-A*11:01 and HLA-C*04:01; HLA-A*25:01 and HLA-C*12:03; HLA-A*02:01 and HLA-C*02:02; HLA-A*24:02 and HLA-C*03:03; HLA-A*30:01 and HLA-C*06:02; HLA-A*02:01 and HLA-C*01:02; HLA-A*11:01 and HLA-C*07:02; HLA-A*03:01 and HLA-C*07:01; HLA-A*23:01 and HLA-C*04:01; HLA-A*24:02 and HLA-C*07:01; HLA-A*31:01 and HLA-C*03:04; HLA-A*33:01 and HLA-C*08:02; HLA-A*02:01 and HLA-C*03:03; HLA-A*11:01 and HLA-C*01:02; HLA-A*01:01 and HLA-C*04:01; HLA-A*03:01 and HLA-C*06:02.

In some embodiments, the cell is homozygous for HLA-A and homozygous for HLA-C. In some embodiments, the HLA-A and HLA-C alleles are HLA-A*01:01 and HLA-C*07:01. In some embodiments, the HLA-A and HLA-C alleles are HLA-A*02:01 and HLA-C*07:02. In some embodiments, the HLA-A and HLA-C alleles are HLA-A*02:01 and HLA-C*05:01. In some embodiments, the HLA-A and HLA-C alleles are HLA-A*03:01 and HLA-C*07:02. In some embodiments, the HLA-A and HLA-C alleles are HLA-A*02:01 and HLA-C*04:01. In some embodiments, the HLA-A and HLA-C alleles are HLA-A*02:01 and HLA-C*03:04. In some embodiments, the HLA-A and HLA-C alleles are HLA-A*01:01 and HLA-C*06:02. In some embodiments, the HLA-A and HLA-C alleles are HLA-A*03:01 and HLA-C*04:01. In some embodiments, the HLA-A and HLA-C alleles are HLA-A*02:01 and HLA-C*07:01. In some embodiments, the HLA-A and HLA-C alleles are HLA-A*24:02 and HLA-C*04:01. In some embodiments, the HLA-A and HLA-C alleles are HLA-A*29:02 and HLA-C*16:01. In some embodiments, the HLA-A and HLA-C alleles are HLA-A*02:01 and HLA-C*06:02. In some embodiments, the HLA-A and HLA-C alleles are HLA-A*24:02 and HLA-C*07:02. In some embodiments, the HLA-A and HLA-C alleles are HLA-A*26:01 and HLA-C*12:03. In some embodiments, the HLA-A and HLA-C alleles are HLA-A*11:01 and HLA-C*04:01. In some embodiments, the HLA-A and HLA-C alleles are HLA-A*25:01 and HLA-C*12:03. In some embodiments, the HLA-A and HLA-C alleles are HLA-A*02:01 and HLA-C*02:02. In some embodiments, the HLA-A and HLA-C alleles are HLA-A*24:02 and HLA-C*03:03. In some embodiments, the HLA-A and HLA-C alleles are HLA-A*30:01 and HLA-C*06:02. In some embodiments, the HLA-A and HLA-C alleles are HLA-A*02:01 and HLA-C*01:02. In some embodiments, the HLA-A and HLA-C alleles are HLA-A*11:01 and HLA-C*07:02. In some embodiments, the HLA-A and HLA-C alleles are HLA-A*03:01 and HLA-C*07:01. In some embodiments, the HLA-A and HLA-C alleles are HLA-A*23:01 and HLA-C*04:01. In some embodiments, the HLA-A and HLA-C alleles are HLA-A*24:02 and HLA-C*07:01. In some embodiments, the HLA-A and HLA-C alleles are HLA-A*31:01 and HLA-C*03:04. In some embodiments, the HLA-A and HLA-C alleles are HLA-A*33:01 and HLA-C*08:02. In some embodiments, the HLA-A and HLA-C alleles are HLA-A*02:01 and HLA-C*03:03. In some embodiments, the HLA-A and HLA-C alleles are HLA-A*11:01 and HLA-C*01:02. In some embodiments, the HLA-A and HLA-C alleles are HLA-A*01:01 and HLA-C*04:01; HLA-A*03:01 and HLA-C*06:02.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3+, CD4+ and CD8+ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell. In some embodiments, the cell is a macrophage. In some embodiments, the lymphocyte is allogeneic.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker. In some embodiments, the T cell is allogeneic.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell.

As used herein, a “central memory T cell” can be defined as an antigen-experienced T cell, and for example, may expresses CD62L and CD45RO. A central memory T cell may be detected as CD62L+ and CD45RO+ by Central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO− CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 or CD20, or B cell mature antigen (“BCMA”), and therefore a B cell is CD19+, or CD20+, or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+ or may have a class-switched B cell receptor (e.g., IgG+, or IgA+). In some embodiments, the B cell is allogeneic.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”). In some embodiments, the mononuclear cell is allogeneic.

Cells used in ACT or tissue regenerative therapy are included, such as stem cells, progenitor cells, and primary cells. Stem cells, for example, include pluripotent stem cells (PSCs); induced pluripotent stem cells (iPSCs); embryonic stem cells (ESCs); mesenchymal stem cells (MSCs, e.g., isolated from bone marrow (BM), peripheral blood (PB), placenta, umbilical cord (UC) or adipose); hematopoietic stem cells (HSCs; e.g. isolated from BM or UC); neural stem cells (NSCs); tissue specific progenitor stem cells (TSPSCs); and limbal stem cells (LSCs). Progenitor and primary cells include mononuclear cells (MNCs, e.g., isolated from BM or PB); endothelial progenitor cells (EPCs, e.g. isolated from BM, PB, and UC); neural progenitor cells (NPCs); and tissue-specific primary cells or cells derived therefrom (TSCs) including chondrocytes, myocytes, and keratinocytes. Cells for organ or tissue transplantations such as islet cells, cardiomyocytes, thyroid cells, thymocytes, neuronal cells, skin cells, and retinal cells are also included.

In some embodiments, the human cell is isolated from a human subject. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

### F. Exemplary Gene Editing Systems

Various suitable gene editing systems may be used to make the engineered cells disclosed herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in gene editing and gene therapy.

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for gene editing in situ, a technique known as gene editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In some embodiments, the gene editing system is a CRISPR/Cas system, including e.g., a CRISPR guide RNA comprising a guide sequence and RNA-guided DNA binding agent, and described further herein.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the presence of a PAM and the sequence of the guide RNA. Exemplary RNA-guided DNA binding agents include Cas cleavases/nickases and inactivated forms thereof (“dCas DNA binding agents”). “Cas nuclease”, as used herein, encompasses Cas cleavases, Cas nickases, and dCas DNA binding agents. The dCas DNA binding agent may be a dead nuclease comprising non-functional nuclease domains (RuvC or HNH domain). In some embodiments the Cas cleavase or Cas nickase encompasses a dCas DNA binding agent modified to permit DNA cleavage, e.g. via fusion with a FokI domain. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases.

As used herein, a “Class 2 Cas nuclease” is a single-chain polypeptide with RNA-guided DNA binding activity. Class 2 Cas nucleases include Class 2 Cas cleavases/nickases (e.g., H840A or D10A variants of Spy Cas9 and D16A and H588A of Nine Cas9, e.g., Nme2 Cas9), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

In some embodiments the gene editing system comprises a base editor comprising a deaminase and an RNA-guided nickase. In some embodiments the gene editing system comprises a base editor comprising a cytidine deaminase and an RNA-guided nickase. In some embodiments, the gene editing system comprises a DNA polymerase. Further description of the gene editing system methods and compositions for use therein are known in the art. See e.g., WO2019/067910, WO2021/188840A1, WO2019/051097, and PCT/US2021/062922 filed Dec. 10, 2021, and U.S. Provisional Application No. 63/275,425 filed Nov. 3, 2021, the contents of each of which are hereby incorporated in their entireties.

Exemplary nucleotide and polypeptide sequences for the gene editing system disclosed herein are provided below in Table 9. Methods for identifying alternate nucleotide sequences encoding polypeptide sequences provided herein, including alternate naturally occurring variants, are known in the art. Sequences with at least 75%, 80%, 85%, 90%, 95%, 96%, 97%, 98%, or 99% identity to any of the nucleic acid sequences, or nucleic acid sequences encoding the amino acid sequences provided herein are also contemplated.

### G. CRISPR Guide RNA

Provided herein are guide sequences useful for modifying a target sequence, e.g., using a guide RNA comprising a disclosed guide sequence with an RNA-guided DNA binding agent (e.g., a CRISPR/Cas system). Guide sequences are shown in Tables 2, 3, 3A, 4, 5A, 5B, 6, 7, and 9A (e.g., SEQ ID NOs: 1-91, 101-185, 301-498, and 500-590), as are the genomic coordinates that these guide RNAs target.

In some embodiments, a gRNA provided herein comprises a guide region (guide sequence) and a conserved region comprising a repeat/anti-repeat region, a hairpin 1 region, and a hairpin 2 region, wherein one or more of the repeat/anti-repeat region, the hairpin 1 region, and the hairpin 2 region are shortened. In some embodiments, the gRNA is from S. pyogenes Cas9 (SpyCas9). In some embodiments, the gRNA is from N. meningitidis Cas9 (NmeCas9).

An exemplary conserved region of an SpyCas9 guide RNA is shown in Table 8A (SEQ ID NO: 600). An exemplary conserved region of an NmeCas9 guide RNA is shown in Table 8B (SEQ ID NO: 3126). The first row shows the numbering of the nucleotides; the second row shows an exemplary sequence; and the third (and fourth) rows show the regions. “Shortened” with respect to an sgRNA means that its conserved region lacks at least one nucleotide shown in Table 8A-8B, as discussed in detail below.

Each of SpyCas9 guide RNAs disclosed herein may further comprise additional nucleotides to form a crRNA, e.g., with the following exemplary nucleotide sequence following the guide sequence at its 3′ end: GUUUUAGAGCUAUGCUGUUUUG (SEQ ID NO: 601) in 5′ to 3′ orientation. In the case of a sgRNA, the above guide sequences may further comprise additional nucleotides (scaffold sequence) to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 602) or GUUUUAGAGCUAGAAAUAGCAAGUUAAAAUAAGGCUAGUCCGUUAUCAACUU GAAAAAGUGGCACCGAGUCGGUGC (SEQ ID NO: 603, which is SEQ ID NO: 602 without the four terminal U's) in 5′ to 3′ orientation. In some embodiments, the four terminal U's of SEQ ID NO: 602 are not present. In some embodiments, only 1, 2, or 3 of the four terminal U's of SEQ ID NO: 602 are present.

In some embodiments, the SpyCas9 sgRNA comprises any one of the SpyCas9 guide sequences (e.g., HLA-B guide sequences of SEQ ID NOs: 1-91 or any one of the HLA-A guide sequences of SEQ ID NOs: 301-428 and 463-511) and additional nucleotides to form a crRNA, e.g., with the following exemplary scaffold nucleotide sequence following the guide sequence at its 3′ end: GUUUUAGAGCUAGAAAUAGCAAGUUAAAAUAAGGCUAGUCCGUUAUCAACUU GGCACCGAGUCGGUGC (SEQ ID NO: 604) in 5′ to 3′ orientation. SEQ ID NO: 604 lacks 8 nucleotides with reference to a wild-type guide RNA conserved sequence: GUUUUAGAGCUAGAAAUAGCAAGUUAAAAUAAGGCUAGUCCGUUAUCAACUU GAAAAAGUGGCACCGAGUCGGUGC (SEQ ID NO: 600). Other exemplary scaffold nucleotide sequences are provided in Table 9. In some embodiments, the sgRNA comprises any one of the guide sequences of SEQ ID NOs: 1-91, 301-428, or 463-511 and additional guide scaffold sequences, in 5′ to 3′ orientation, in Table 9, including modified versions of the scaffold sequences, as shown.

In some embodiments, a gRNA provided herein comprises a guide region and a conserved region comprising a repeat/anti-repeat region, a hairpin 1 region, and a hairpin 2 region, wherein one or more of the repeat/anti-repeat region, the hairpin 1 region, and the hairpin 2 region are shortened. In some embodiments, the gRNA is from N. meningitidis Cas9 (NmeCas9).

In some embodiments, the guide RNA comprises a modified sgRNA. In some embodiments, the sgRNA comprises any one of the modification patterns of the modified sgRNA sequences provided in Tables 2, 3, 3A, 4, 5A, 5B, 6, 7, 8A, 8B, 9, and 9A. In some embodiments, the conserved region comprises any one of modified conserved region Nine guide RNA motifs in Tables 8B, 9, and 9A, and wherein the conserved region is 3′ of the guide region (guide sequence). In some embodiments, the conserved region comprises a modified sequence comprising any one of SEQ ID NOs: 715-723, and wherein the conserved region is 3′ of the guide region (guide sequence). In some embodiments, the guide RNA comprises a nucleotide sequence selected from any one of SEQ ID NOs: 708 and 712-714, where the N's represent collectively any guide sequence disclosed herein, including the guide sequences provided in Tables 3, 3A, 5A, 7, and 9A. In certain embodiments, the N's represent collectively a guide sequence that is at least 80%, 85%, 90%, 95%, or 100% identical to or complementary to any one of the guide sequences provided in Tables 3, 3A, 5A, 7, and 9A. In certain embodiments, the N's represent collectively any one of the guide sequences provided in Tables 3, 3A, 5A, 7, and 9A. In certain embodiments, when the N's represent collectively a guide sequence, within (N)₂₀₋₂₅, each N of the (N)₂₀₋₂₅ may be independently modified, e.g., modified with a 2′-OMe modification, optionally further with a PS modification, particularly at 1, 2, or 3 terminal nucleotides. In certain embodiments, the (N)₂₀₋₂₅ has the following sequence and modification pattern:

mN*mN*mN*mNmNNNmNmNNmNNmNNNNNmNNNNmNNN.

An exemplary conserved region of an NmeCas9 single guide RNA (Nine sgRNA) is shown in Table 8B (SEQ ID NO: 3126). The first row shows the numbering of the nucleotides; the second row shows an exemplary sequence; and the third (and fourth) rows show the regions. “Shortened” with respect to an sgRNA means that its conserved region lacks at least one nucleotide shown in Table 8B, as discussed in detail below.

In some embodiments, the NmeCas9 sgRNA comprises any one of the Nine Cas9 guide sequences disclosed herein (e.g., SEQ ID NOs: 101-185) and additional nucleotides to form a crRNA, e.g., with the following exemplary scaffold nucleotide sequence following the guide sequence at its 3′ end:

(SEQ ID NO: 699)
GUUGUAGCUCCCUUUCUCAUUUCGGAAACGAAAUGAGAACCGUUGCUAC
AAUAAGGCCGUCUGAAAAGAUGUGCCGCAACGCUCUGCCCCUUAAAGCU
UCUGCUUUAAGGGGCAUCGUUUA.

In some embodiments, the NmeCas9sgRNA comprises any one of the guide sequences of SEQ ID NOs: 101-185 and additional nucleotides to form a crRNA with the following nucleotide sequence following the guide sequence at its 3′ end:

(SEQ ID NO: 701)
GUUGUAGCUCCCUGAAACCGUUGCUACAAUAAGGCCGUCGAAAGAUGU
GCCGCAACGCUCUGCCUUCUGGCAUCGUU;
(SEQ ID NO: 702)
GUUGUAGCUCCCUGAAACCGUUGCUACAAUAAGGCCGUCGAAAGAUGU
GCCGCAACGCUCUGCCUUCUGGCAUCGUUUAUU;
(SEQ ID NO: 703)
GUUGUAGCUCCCUGGAAACCCGUUGCUACAAUAAGGCCGUCGAAAGA
UGUGCCGCAACGCUCUGCCUUCUGGCAUCGUUUAUU.

In some embodiments, the guide RNA is a chemically modified guide RNA. In some embodiments, the guide RNA is a chemically modified single guide RNA. The chemically modified guide RNAs may comprise one or more of the modifications as shown in Tables 2-7. The chemically modified guide RNAs may comprise one or more of modified nucleotides of any one of SEQ ID NOs: 705-714.

In some embodiments, the guide RNA is a sgRNA comprising the modification pattern shown in SEQ ID NO: 705-714.

In some embodiments, the guide RNA comprises a sgRNA comprising the modification pattern shown in SEQ ID NO: 705, 708, 711, 712, 713, or 714. In some embodiments, the guide RNA comprises a sgRNA comprising the modified nucleotides of SEQ ID NO: 705, 708, 711, 712, 713, or 714, including a guide sequence disclosed herein (e.g., SEQ ID NOs: 1-91). In some embodiments, the guide RNA is a sgRNA comprising a sequence of SEQ ID NO: 705, 708, 711, 712, 713, or 714 or a sequence that is at least 99%, 98%, 97%, 96%, 95%, 94%, 93%, 92%, 91%, or 90% identical to SEQ ID NO: 705, 708, 711, 712, 713, or 714.

The guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, a crRNA or trRNA sequence may be referred to as a “scaffold” or “conserved portion” of a guide RNA.

In each of the compositions, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown in Tables 2-3, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Tables 2-3, covalently linked to a trRNA. The sgRNA may comprise 17, 18, 19, or 20 contiguous nucleotides of a guide sequence shown in Tables 2-3. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition comprising one or more guide RNAs comprising a guide sequence of any one in Tables 2-3 (for HLA-B SpyCas9 and NmeCas9 guides) and Tables 4, 5B and 6 (for HLA-A SpyCas9 guides) and Table 5A and 7 (for HLA-A NmeCas9 guides) is provided. In some embodiments, a composition comprising one or more guide RNAs comprising a guide sequence of any one in Tables 2-3 is provided, wherein the nucleotides of SEQ ID NO: 601-604 follow the guide sequence at its 3′ end. In some embodiments, the one or more guide RNAs comprising a guide sequence of any one in Tables 2-3 (for HLA-B SpyCas9 and NmeCas9 guides) and Tables 4, 5B and 6 (for HLA-A SpyCas9 guides), wherein the nucleotides of SEQ ID NO: 601-604 follow the guide sequence at its 3′ end, is modified according to the modification pattern of any one of SEQ ID NOs: 3003, 3007-3009, and 3011-3014.

In some embodiments, a composition comprising one or more guide RNAs comprising a guide sequence of any one in Tables 2-3 (for HLA-B SpyCas9 and NmeCas9 guides) and Tables 4, 5B and 6 (for HLA-A SpyCas9 guides) is provided. In one aspect, a composition comprising one or more gRNAs is provided, comprising a guide sequence that is at least 99%, 98%, 97%, 96%, 95%, 94%, 93%, 92%, 91%, or 90% identical to any of the nucleic acids of SEQ ID NOs: 1-91, 301-428, and 463-511.

In other embodiments, a composition is provided that comprises at least one, e.g., at least two gRNA's comprising guide sequences selected from any two or more of the guide sequences shown in Tables 2-3 (for HLA-B SpyCas9 and NmeCas9 guides) and Tables 4, 5B and 6 (for HLA-A SpyCas9 guides). In some embodiments, the composition comprises at least two gRNA's that each comprise a guide sequence at least 99%, 98%, 97%, 96%, 95%, 94%, 93%, 92%, 91%, or 90% identical to any of the guide sequences shown in Tables 2-3 (for HLA-B SpyCas9 and NmeCas9 guides) and Tables 4, 5B, and 6 (for HLA-A SpyCas9 and NmeCas9 guides).

In some embodiments, the guide RNA compositions of the present invention are designed to recognize (e.g., hybridize to) a target sequence in HLA-B. For example, the HLA-B target sequence may be recognized and cleaved by a provided Cas cleavase comprising a guide RNA. In some embodiments, an RNA-guided DNA binding agent, such as a Cas cleavase, may be directed by a guide RNA to a target sequence in HLA-B, where the guide sequence of the guide RNA hybridizes with the target sequence and the RNA-guided DNA binding agent, such as a Cas cleavase, cleaves the target sequence.

In some embodiments, the guide RNA compositions of the present invention are designed to recognize (or hybridize to) a target sequence in HLA-A and HLA-B. For example, the HLA-A and HLA-B target sequence may be recognized and cleaved by a provided Cas cleavase comprising a guide RNA. In some embodiments, an RNA-guided DNA binding agent, such as a Cas cleavase, may be directed by a guide RNA to a target sequence in HLA-A and HLA-B, where the guide sequence of the guide RNA hybridizes with the target sequence and the RNA-guided DNA binding agent, such as a Cas cleavase, cleaves the target sequence.

In some embodiments, the selection of the one or more HLA-B guide RNAs is determined based on target sequences within HLA-B. In some embodiments, the compositions comprising one or more guide sequences comprise a guide sequence that is complementary to the corresponding genomic region shown in Tables 2-3, according to coordinates from human reference genome hg38. Guide sequences of further embodiments may be complementary to sequences in the close vicinity of the genomic coordinate listed in any of the Tables 2-3 within HLA-B. For example, guide sequences of further embodiments may be complementary to sequences that comprise 10 contiguous nucleotides ±10 nucleotides of a genomic coordinate listed in Tables 2-3.

In some embodiments, the selection of the one or more HLA-A guide RNAs is determined based on target sequences within HLA-A. In some embodiments, the compositions comprising one or more guide sequences comprise a guide sequence that is complementary to the corresponding genomic region shown in Tables 4-7, according to coordinates from human reference genome hg38. Guide sequences of further embodiments may be complementary to sequences in the close vicinity of the genomic coordinate listed in any of the Tables 4-7 within HLA-A. For example, guide sequences of further embodiments may be complementary to sequences that comprise 10 contiguous nucleotides ±10 nucleotides of a genomic coordinate listed in Tables 4-7. Without being bound by any particular theory, modifications (e.g., frameshift mutations resulting from indels occurring as a result of a nuclease-mediated DSB) in certain regions of the target gene may be less tolerable than mutations in other regions, thus the location of a DSB is an important factor in the amount or type of protein knockdown that may result. In some embodiments, a gRNA complementary or having complementarity to a target sequence within the target gene used to direct an RNA-guided DNA binding agent to a particular location in the target gene.

In some embodiments, the guide sequence is at least 99%, 98%, 97%, 96%, 95%, 94%, 93%, 92%, 91%, 90%, 85%, or 80% identical to a target sequence present in the target gene. In some embodiments, the guide sequence is at least 99%, 98%, 97%, 96%, 95%, 94%, 93%, 92%, 91%, 90%, 85%, or 80% identical to a target sequence present in the human HLA-A or HLA-B gene.

In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

### H. Modified gRNAs and mRNAs

In some embodiments, the gRNA (e.g., sgRNA, short-sgRNA, dgRNA, or crRNA) is modified. The term “modified” or “modification” in the context of a gRNA described herein includes, the modifications described above, including, for example, (a) end modifications, e.g., 5′ end modifications or 3′ end modifications, including 5′ or 3′ protective end modifications, (b) nucleobase (or “base”) modifications, including replacement or removal of bases, (c) sugar modifications, including modifications at the 2′, 3′, or 4′ positions, (d) internucleoside linkage modifications, and (e) backbone modifications, which can include modification or replacement of the phosphodiester linkages or the ribose sugar. A modification of a nucleotide at a given position includes a modification or replacement of the phosphodiester linkage immediately 3′ of the sugar of the nucleotide. Thus, for example, a nucleic acid comprising a phosphorothioate between the first and second sugars from the 5′ end is considered to comprise a modification at position 1. The term “modified gRNA” generally refers to a gRNA having a modification to the chemical structure of one or more of the base, the sugar, and the phosphodiester linkage or backbone portions, including nucleotide phosphates, all as detailed and exemplified herein.

Further description and exemplary patterns of modifications are provided in Table 1 of WO2019/237069 published Dec. 12, 2019, the entire contents of which are incorporated herein by reference.

In some embodiments, a gRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, 2′-H, inosine, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

In some embodiments, the guide sequence (or guide region) of a gRNA comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications. A modified guide region YA site comprises a YA modification.

In some embodiments, a modified guide region YA site is within 20, 19, 18, 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, a sgRNA can comprise a 5′ end modification as described herein and further comprise a modified guide region YA site. Alternatively, a sgRNA can comprise an unmodified 5′ end and a modified guide region YA site. Alternatively, a short-sgRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above. The guide region of a gRNA may be modified according to any embodiment comprising a modified guide region set forth herein. Any embodiments set forth elsewhere in this disclosure may be combined to the extent feasible with any of the foregoing embodiments.

In some embodiments, the 5′ or 3′ terminus regions of a gRNA are modified.

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, the 3′ end modification comprises or further comprises any one or more of the following: a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof. In some embodiments, the 3′ end modification comprises or further comprises modifications of 1, 2, 3, 4, 5, 6, or 7 nucleotides at the 3′ end of the gRNA. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides. In some embodiments, the 3′ end modification comprises or further comprises PS linkages between any one or more of the last 2, 3, 4, 5, 6, or 7 nucleotides. In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified. In some embodiments, a gRNA is provided comprising a 3′ protective end modification. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the gRNA does not comprise a 3′ tail.

In some embodiments, the 5′ terminus region is modified, for example, the first 1, 2, 3, 4, 5, 6, or 7 nucleotides of the gRNA are modified. Throughout, this modification may be referred to as a “5′ end modification”. In some embodiments, the first 1, 2, 3, 4, 5, 6, or 7 nucleotides of the 5′ terminus region comprise more than one modification. In some embodiments, at least one of the terminal (i.e., first) 1, 2, 3, 4, 5, 6, or 7 nucleotides at the 5′ end are modified. In some embodiments, both the 5′ and 3′ terminus regions (e.g., ends) of the gRNA are modified. In some embodiments, only the 5′ terminus region of the gRNA is modified. In some embodiments, only the 3′ terminus region (plus or minus a 3′ tail) of the conserved portion of a gRNA is modified. In some embodiments, the gRNA comprises modifications at 1, 2, 3, 4, 5, 6, or 7 of the first 7 nucleotides at a 5′ terminus region of the gRNA. In some embodiments, the gRNA comprises modifications at 1, 2, 3, 4, 5, 6, or 7 of the 7 terminal nucleotides at a 3′ terminus region. In some embodiments, 2, 3, or 4 of the first 4 nucleotides at the 5′ terminus region, or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds. In some embodiments, the modification to the 5′ terminus or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed.

In some embodiments, a gRNA is provided comprising a 5′ end modification and a 3′ end modification. In some embodiments, the gRNA comprises modified nucleotides that are not at the 5′ or 3′ ends.

In some embodiments, a sgRNA is provided comprising an upper stem modification, wherein the upper stem modification comprises a modification to any one or more of US1-US12 in the upper stem region. In some embodiments, a sgRNA is provided comprising an upper stem modification, wherein the upper stem modification comprises a modification of at least 1, 2, 3, 4, 5, 6, 7, 8, 9, 10, 11, or all 12 nucleotides in the upper stem region. In some embodiments, an sgRNA is provided comprising an upper stem modification, wherein the upper stem modification comprises 1, 2, 3, 4, or 5 YA modifications in a YA site. In some embodiments, the upper stem modification comprises a 2′-OMe modified nucleotide, a 2′-O-moe modified nucleotide, a 2′-F modified nucleotide, or combinations thereof. Other modifications described herein, such as a 5′ end modification or a 3′ end modification may be combined with an upper stem modification.

In some embodiments, the sgRNA comprises a modification in the hairpin region. In some embodiments, the hairpin region modification comprises at least one modified nucleotide selected from a 2′-O-methyl (2′-OMe) modified nucleotide, a 2′-fluoro (2′-F) modified nucleotide, or combinations thereof. In some embodiments, the hairpin region modification is in the hairpin 1 region. In some embodiments, the hairpin region modification is in the hairpin 2 region. In some embodiments, the hairpin modification comprises 1, 2, or 3 YA modifications in a YA site. In some embodiments, the hairpin modification comprises at least 1, 2, 3, 4, 5, or 6 YA modifications. Other modifications described herein, such as an upper stem modification, a 5′ end modification, or a 3′ end modification may be combined with a modification in the hairpin region.

In some embodiments, a gRNA comprises a substituted and optionally shortened hairpin 1 region, wherein at least one of the following pairs of nucleotides are substituted in the substituted and optionally shortened hairpin 1 with Watson-Crick pairing nucleotides: H1-1 and H1-12, H1-2 and H1-11, H1-3 and H1-10, or H1-4 and H1-9. “Watson-Crick pairing nucleotides” include any pair capable of forming a Watson-Crick base pair, including A-T, A-U, T-A, U-A, C-G, and G-C pairs, and pairs including modified versions of any of the foregoing nucleotides that have the same base pairing preference. In some embodiments, the hairpin 1 region lacks any one or two of H1-5 through H1-8. In some embodiments, the hairpin 1 region lacks one, two, or three of the following pairs of nucleotides: H1-1 and H1-12, H1-2 and H1-11, H1-3 and H1-10 or H1-4 and H1-9. In some embodiments, the hairpin 1 region lacks 1-8 nucleotides of the hairpin 1 region. In any of the foregoing embodiments, the lacking nucleotides may be such that the one or more nucleotide pairs substituted with Watson-Crick pairing nucleotides (H1-1 and H1-12, H1-2 and H1-11, H1-3 and H1-10, or H1-4 and H1-9) form a base pair in the gRNA.

In some embodiments, the gRNA further comprises an upper stem region lacking at least 1 nucleotide, e.g., any of the shortened upper stem regions indicated in Table 7 of WO/2021/119275, the contents of which are hereby incorporated by reference in its entirety, or described elsewhere herein, which may be combined with any of the shortened or substituted hairpin 1 regions described herein.

In some embodiments, an sgRNA provided herein is a short-single guide RNAs (short-sgRNAs), e.g., comprising a conserved portion of an sgRNA comprising a hairpin region, wherein the hairpin region lacks at least 5-10 nucleotides or 6-10 nucleotides. In some embodiments, the 5-10 nucleotides or 6-10 nucleotides are consecutive.

In some embodiments, a short-sgRNA lacks at least nucleotides 54-58 (AAAAA) of the conserved portion of a spyCas9 sgRNA. In some embodiments, a short-sgRNA is a non-spyCas9 sgRNA that lacks nucleotides corresponding to nucleotides 54-58 (AAAAA) of the conserved portion of a spyCas9 as determined, for example, by pairwise or structural alignment.

In some embodiments, the short-sgRNA described herein comprises a conserved portion comprising a hairpin region, wherein the hairpin region lacks 5, 6, 7, 8, 9, 10, 11, or 12 nucleotides. In some embodiments, the lacking nucleotides are 5-10 lacking nucleotides or 6-10 lacking nucleotides. In some embodiments, the lacking nucleotides are consecutive. In some embodiments, the lacking nucleotides span at least a portion of hairpin 1 and a portion of hairpin 2. In some embodiments, the 5-10 lacking nucleotides comprise or consist of nucleotides 54-58, 54-61, or 53-60 of SEQ ID NO: 600.

In some embodiments, the short-sgRNA described herein further comprises a nexus region, wherein the nexus region lacks at least one nucleotide (e.g., 1, 2, 3, 4, 5, 6, 7, 8, 9, or 10 nucleotides in the nexus region). In some embodiments, the short-sgRNA lacks each nucleotide in the nexus region.

In some embodiments, the SpyCas9 short-sgRNA described herein comprises a sequence of

(SEQ ID NO: 3002)
NNNNNNNNNNNNNNNNNNNNGUUUUAGAGCUAGAAAUAGCAAGUUAAAA
UAAGGCUAGUCCGUUAUCACGAAAGGGCACCGAGUCGGUGCU.

In some embodiments, the short-sgRNA described herein comprises a modification pattern as shown in SEQ ID NO: 3003: mN*mN*mN*NNNNNNNNNNNNNNNNNGUUUUAGAmGmCmUmAmGmAmAmAmU mAmGmCAAGUUAAAAUAAGGCUAGUCCGUUAUCACGAAAGGGCACCGAGUCG GmUmGmC*mU (SEQ ID NO: 3003), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides.

In certain embodiments, using SEQ ID NO: 600 (“Exemplary SpyCas9 sgRNA-1”) as an example, the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 6-8 nucleotides, preferably 6 nucleotides; and
      a. one or more of positions H1-1, H1-2, or H1-3 is deleted or substituted relative to Exemplary SpyCas9 sgRNA-1 (SEQ ID NO: 600) or
      b. one or more of positions H1-6 through H1-10 is substituted relative to Exemplary SpyCas9 sgRNA-1 (SEQ ID NO: 600); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 600); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 600); or
C. a substitution relative to Exemplary SpyCas9 sgRNA-1 (SEQ ID NO: 600) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. Exemplary SpyCas9 sgRNA-1 (SEQ ID NO: 600) with an upper stem region, wherein the upper stem modification comprises a modification to any one or more of US1-US12 in the upper stem region, wherein
   1. the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or
   2. the modified nucleotide optionally includes a 2′-OMe modified nucleotide.

In certain embodiments, Exemplary SpyCas9 sgRNA-1, or an sgRNA, such as an sgRNA comprising Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide, or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In some embodiments, the NmeCas9 gRNA described herein further comprises a nexus region, wherein the nexus region lacks at least one nucleotide.

In some embodiments, the HLA-B NmeCas9 sgRNA chosen from SEQ ID NOs 101-185 comprises a conserved portion comprising a repeat/anti-repeat region, a hairpin 1 region, and a hairpin 2 region, wherein one or more of the repeat/anti-repeat region, the hairpin 1 region, and the hairpin 2 region are shortened.

In some embodiments, the guide RNA is a Nine sgRNA comprising a conserved portion comprising a repeat/anti-repeat region, a hairpin 1 region, and a hairpin 2 region, wherein one or more of the repeat/anti-repeat region, the hairpin 1 region, and the hairpin 2 region are shortened. In some embodiments, the sgRNA described herein further comprises a guide region and a conserved region, wherein the conserved region comprises one or more of: (a) a shortened repeat/anti-repeat region, wherein the shortened repeat/anti-repeat region lacks 2-24 nucleotides, wherein (i) one or more of nucleotides 37-48 and 53-64 is deleted and optionally one or more of nucleotides 37-64 is substituted relative to SEQ ID NO: 3126; and (ii) nucleotide 36 is linked to nucleotide 65 by at least 2 nucleotides; or (b) a shortened hairpin 1 region, wherein the shortened hairpin 1 lacks 2-10, optionally 2-8 nucleotides, wherein one or more of nucleotides 82-86 and 91-95 is deleted and optionally one or more of positions 82-96 is substituted relative to SEQ ID NO: 3126; and nucleotide 81 is linked to nucleotide 96 by at least 4 nucleotides; or (c) a shortened hairpin 2 region, wherein the shortened hairpin 2 lacks 2-18, optionally 2-16 nucleotides, wherein (i) one or more of nucleotides 113-121 and 126-134 is deleted and optionally one or more of nucleotides 113-134 is substituted relative to SEQ ID NO: 3126; and (ii) nucleotide 112 is linked to nucleotide 135 by at least 4 nucleotides; wherein one or both nucleotides 144-145 are optionally deleted relative to SEQ ID NO: 3126; and wherein at least 10 nucleotides are modified nucleotides.

In some embodiment, the gRNA disclosed herein is a sgRNA.

In some embodiments, in the guide sequence, nucleotides 1-4 are modified nucleotides. In some embodiments, in the guide sequence, nucleotides 5, 8, 9, 11, 13, 18, and 22 are modified nucleotides. In some embodiments, in the guide sequence, nucleotides 1-5, 8, 9, 11, 13, 18, and 22 are modified nucleotides. In some embodiments, the modified nucleotides are 2′-O-methyl (2′-O-Me) modified nucleotides. In some embodiments, in the guide sequence, nucleotide 1 is linked to nucleotide 2 by a phosphorothioate (PS) linkage, nucleotide 2 is linked to nucleotide 3 by a PS linkage, and/or nucleotide 3 is linked to nucleotide 4 by a PS linkage.

In some embodiments, one or both nucleotides 144-145 are deleted relative to SEQ ID NO: 3126.

In some embodiments, at least 10 nucleotides of the conserved region are modified nucleotides.

In some embodiments, a repeat/anti-repeat region of a gRNA is a shortened repeat/anti-repeat region lacking 2-24 nucleotides, e.g., any of the repeat/anti-repeat regions indicated in the numbered embodiments above or Tables 3, 3A, 5A, 7, and 9A or described elsewhere herein, which may be combined with any of the shortened hairpin 1 region or hairpin 2 region described herein, including but not limited to combinations indicated in the numbered embodiments above and represented in the sequences of Tables 3, 3A, 5A, 7, and 9A or described elsewhere herein. In some embodiments, one or more of positions 49-52, 87-90, or 122-125 is substituted relative to SEQ ID NO: 3126. In some embodiments, all of positions 49-52, 87-90, or 122-125 are substituted relative to SEQ ID NO: 3126. In some embodiments, the 3′ tail provided in Tables 3, 3A, 5A, 7, and 9A or described herein is deleted.

In some embodiments, the shortened repeat/anti-repeat region of the gRNA lacks 18 nucleotides. In some embodiments, the shortened repeat/anti-repeat region of the gRNA lacks 22 nucleotides.

In some embodiments, in the shortened repeat/anti-repeat region of the gRNA, nucleotide 36 is linked to nucleotide 65 by 6 nucleotides. In some embodiments, in the shortened repeat/anti-repeat region of the gRNA, nucleotide 36 is linked to nucleotide 65 by 7 nucleotides. In some embodiments, in the shortened repeat/anti-repeat region of the gRNA, nucleotide 36 is linked to nucleotide 65 by 8 nucleotides. In some embodiments, in the shortened repeat/anti-repeat region of the gRNA, nucleotide 36 is linked to nucleotide 65 by 9 nucleotides. In some embodiments, in the shortened repeat/anti-repeat region of the gRNA, nucleotide 36 is linked to nucleotide 65 by 10 nucleotides.

In some embodiments, in the shortened repeat/anti-repeat region of the gRNA, nucleotides 38-48 and 53-63 are deleted relative to SEQ ID NO: 3126. In some embodiments, in the shortened repeat/anti-repeat region of the gRNA, nucleotides 38, 41-48, 53-60, and 63 are deleted relative to SEQ ID NO: 3126.

In some embodiments, in the shortened repeat/anti-repeat region of the gRNA, nucleotide 36 is linked to nucleotide 65 by 6 nucleotides. In some embodiments, in the shortened repeat/anti-repeat region of the gRNA, nucleotides 38-48 and 53-63 are deleted relative to SEQ ID NO: 3126, and nucleotide 36 is linked to nucleotide 65 by nucleotides 37, 49-52, and 64.

In some embodiments, in the shortened repeat/anti-repeat region of the gRNA, nucleotide 36 is linked to nucleotide 65 by 10 nucleotides. In some embodiments, in the shortened repeat/anti-repeat region of the gRNA, nucleotides 38, 41-48, 53-60, and 63 are deleted relative to SEQ ID NO: 3126, and nucleotide 36 is linked to nucleotide 65 by nucleotides 37, 39, 40, 49-52, 61, 62, and 64.

In some embodiments, all of nucleotides 38-48 and nucleotides 53-63 of the upper stem of the shortened repeat/anti-repeat region are deleted relative to SEQ ID NO: 3126.

In some embodiments, all of nucleotides 39-48 and nucleotides 53-62 of the upper stem of the shortened repeat/anti-repeat region are deleted relative to SEQ ID NO: 3126, and nucleotides 38 and 63 is substituted.

In some embodiments, the shortened repeat/anti-repeat region has 14 modified nucleotides. In some embodiments, the shortened repeat/anti-repeat region has 15 modified nucleotides. In some embodiments, the shortened repeat/anti-repeat region has 16 modified nucleotides. In some embodiments, the shortened repeat/anti-repeat region has 17 modified nucleotides. In some embodiments, the shortened repeat/anti-repeat region has 18 modified nucleotides. In some embodiments, the shortened repeat/anti-repeat region has 19 modified nucleotides. In some embodiments, the shortened repeat/anti-repeat region has 20 modified nucleotides. In some embodiments, in the shortened repeat/anti-repeat region, nucleotides 25, 29, 30, 31, 32, 37, 49-52, 64, 65, 69, 70, and 73 are modified nucleotides. In some embodiments, the modified nucleotides are 2′-O-Me modified nucleotides.

In some embodiments, between the shortened repeat/anti-repeat region and the shortened hairpin 1 region, nucleotide 76 is linked to nucleotide 77 by a PS linkage.

In some embodiments, the shortened hairpin 1 region lacks 2 nucleotides. In some embodiments, the shortened hairpin 1 region lacks 21 nucleotides. In some embodiments, the shortened hairpin 1 region lacks 2 nucleotides, and nucleotides 86 and 91 are deleted relative to SEQ ID NO: 3126. In some embodiments, the shortened hairpin 1 region lacks 2 nucleotides, and nucleotides 85 and 92 are deleted relative to SEQ ID NO: 3126. In some embodiments, in the shortened hairpin 1 region, nucleotide 81 is linked to nucleotide 96 by 12 nucleotides. In some embodiments, in the shortened hairpin 1 region, nucleotide 81 is linked to nucleotide 96 by 12 nucleotides. In some embodiments, in the shortened hairpin 1 region, nucleotides 86 and 91 are deleted relative to SEQ ID NO: 3126, and nucleotide 81 is linked to nucleotide 96 by nucleotides 82-85, 87-90, and 92-95. In some embodiments, in the shortened hairpin 1 region, nucleotides 85 and 92 are deleted relative to SEQ ID NO: 3126, and nucleotide 81 is linked to nucleotide 96 by nucleotides 82-84, 86-91, and 93-95.

In some embodiments, the shortened hairpin 1 region has a duplex portion of 7 base paired nucleotides in length. In some embodiments, the shortened hairpin 1 region has a duplex portion of 8 base paired nucleotides in length.

In the stem of the shortened hairpin 1 region is seven base paired nucleotides in length. In some embodiments, nucleotides 85-86 and nucleotides 91-92 of the shortened hairpin 1 region are deleted.

In some embodiments, the shortened hairpin 1 region has 13 modified nucleotides. In some embodiments, in the shortened hairpin 1 region, nucleotides 80, 81, 83, 84, 85, 87-90, 92-94, and 99 are modified nucleotides. In some embodiments, the modified nucleotides are 2′-O-Me modified nucleotides.

In some embodiments, between the shortened hairpin 1 region and the shortened hairpin 2 region, nucleotide 101 is a modified nucleotide. In some embodiments, the modified nucleotide is a 2′-O-Me modified nucleotide.

In some embodiments, the shortened hairpin 2 lacks 18 nucleotides. In some embodiments, the shortened hairpin 2 has 24 nucleotides. In some embodiments, in the shortened hairpin 2 nucleotides 113-121 and 126-134 are deleted relative to SEQ ID NO: 3126. In some embodiments, the shortened hairpin 2 lacks 18 nucleotides, and nucleotides 113-121 and 126-134 are deleted relative to SEQ ID NO: 3126. In some embodiments, in the shortened hairpin 2 region, nucleotide 112 is linked to nucleotide 135 by 4 nucleotides. In some embodiments, in the shortened hairpin 2 region, nucleotides 113-121 and 126-134 are deleted relative to SEQ ID NO: 3126 and nucleotide 112 is linked to nucleotide 135 by nucleotides 122-125. In some embodiments, in the shortened hairpin 2 region, nucleotides 112-120 and 127-135 are deleted relative to SEQ ID NO: 3126. In some embodiments, the shortened hairpin 2 region lacks 18 nucleotides, and nucleotides 112-120 and 127-135 are deleted relative to SEQ ID NO: 3126.

In some embodiments, the shortened repeat/anti-repeat region has a length of 28 nucleotides. In some embodiments, the shortened repeat/anti-repeat region has a length of 32 nucleotides.

In some embodiments, the upper stem of the shortened repeat/anti-repeat region comprises no more than one base pair. In some embodiments, the upper stem of the shortened repeat/anti-repeat region comprises no more than three base pairs.

In some embodiments, the shortened hairpin 2 region has 8 modified nucleotides. In some embodiments, the shortened hairpin 2 region has 9 modified nucleotides. In some embodiments, the shortened hairpin 2 region has 13 modified nucleotides. In some embodiments, in the shortened hairpin 2 region, nucleotides 104, 110, 111, 122-125, 142, and 143 are modified nucleotides. In some embodiments, in the shortened hairpin 2 region, nucleotides 104, 106-111, 122-125, 142, and 143 are modified nucleotides. In some embodiments, the modified nucleotides are 2′-O-Me modified nucleotides.

In some embodiments, in the shortened hairpin 2 region, nucleotide 141 is linked to nucleotide 142 by a PS linkage, and/or nucleotide 142 is linked to nucleotide 143 by a PS linkage.

In some embodiments, a guide RNA (gRNA) comprises a guide region and a conserved region, the conserved region comprising:
(a) a shortened repeat/anti-repeat region, wherein the shortened repeat/anti-repeat region lacks 18-22 nucleotides relative to SEQ ID NO: 3126, wherein
   (i) nucleotides 38-48 and 53-63 are deleted; and
   (ii) nucleotide 36 is linked to nucleotide 65 by 6-10 nucleotides;
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 3126; and
(c) a shortened hairpin 2 region, wherein the shortened hairpin 2 lacks 18 nucleotides, wherein nucleotides 113-121 and 126-134 are deleted relative to SEQ ID NO: 3126; and wherein nucleotides 144-145 are deleted relative to SEQ ID NO: 3126; wherein at least 10 nucleotides are modified nucleotides.

In some embodiments, a guide RNA (gRNA) comprises a guide region and a conserved region, the conserved region comprising:
(a) a shortened repeat/anti-repeat region, wherein the shortened repeat/anti-repeat region lacks 18-22 nucleotides relative to SEQ ID NO: 3126, wherein
   (i) nucleotides 38, 41-48, 53-60, and 63 are deleted; and
   (ii) nucleotide 36 is linked to nucleotide 65 by 6-10 nucleotides;
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 3126;
(c) a shortened hairpin 2 region, wherein the shortened hairpin 2 lacks 18 nucleotides, wherein nucleotides 113-121 and 126-134 are deleted relative to SEQ ID NO: 3126; and
wherein nucleotides 144-145 are deleted relative to SEQ ID NO: 3126;
wherein at least 10 nucleotides are modified nucleotides.

In some embodiments, a guide RNA (gRNA) is provided, the gRNA comprising a guide region and a conserved region, the conserved region comprising one or more of:
(a) a shortened repeat/anti-repeat region, wherein the shortened repeat/anti-repeat region lacks 18-22 nucleotides relative to SEQ ID NO: 3126, wherein
   (i) nucleotides 37-48 and 53-64 are deleted; and
   (ii) nucleotide 36 is linked to nucleotide 65 by 6-10 nucleotides; or
(b) a shortened hairpin 1 region, wherein the shortened hairpin 1 lacks 2 nucleotides, wherein nucleotides 86 and 91 are deleted or nucleotides 85 and 92 are deleted relative to SEQ ID NO: 3126; or
(c) a shortened hairpin 2 region, wherein the shortened hairpin 2 lacks 18 nucleotides, wherein nucleotides 113-121 and 126-134 are deleted relative to SEQ ID NO: 3126; and
wherein nucleotides 144-145 are deleted relative to SEQ ID NO: 3126;
wherein at least 10 nucleotides are modified nucleotides.

In further embodiments, the shortened repeat/anti-repeat region, wherein the shortened repeat/anti-repeat region lacks 22 nucleotides relative to SEQ ID NO: 3126. In further embodiments, nucleotide 36 is linked to nucleotide 65 by a sequence comprising the nucleotide sequence UGAAAC. In further embodiments, the nucleotide 36 is linked to nucleotide 65 by 10 nucleotides. In further embodiments, the nucleotide 36 is linked to nucleotide 65 by a sequence comprising the nucleotide sequence UUCGAAAGAC (SEQ ID NO: 3122).

In some embodiments, a guide RNA (gRNA) is provided, the gRNA comprising: a guide sequence comprising:
2′-O-Me modified nucleotides at the first four nucleotides 1-4;
PS linkages between nucleotides 1-2, 2-3, and 3-4; and
2′-O-Me modified nucleotides at nucleotides 5, 8, 9, 11, 13, 18, and 22 of the guide sequence;
   a shortened repeat/anti-repeat region, wherein nucleotides 38-48 and 53-63 are deleted relative to SEQ ID NO: 3126, comprising:
2′-O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
   a shortened hairpin 1 region, wherein nucleotides 86 and 91 are deleted relative to SEQ ID NO: 3126, comprising:
2′-O-Me modified nucleotides at nucleotides 80, 81, 83, 84, 85, 87-90, 92-94, and 99; 2′-O-Me modified nucleotide at nucleotide 101 between the shortened hairpin 1 region and the shortened hairpin 2 region;
   a shortened hairpin 2 region, wherein nucleotides 112-120 and 127-135 are deleted relative to SEQ ID NO: 3126, comprising:
2′-O-Me modified nucleotides at nucleotides 104, 110, 111, 122-125, 142, and 143,
PS linkages between nucleotides 141-142 and 142-143,
   wherein one or both nucleotides 144-145 are optionally deleted relative to SEQ ID NO: 3126.

In some embodiments, a guide RNA (gRNA) is provided, the gRNA comprising: a guide sequence comprising:
2′-O-Me modified nucleotides at the first four nucleotides 1-4;
PS linkages between nucleotides 1-2, 2-3, and 3-4; and
2′-O-Me modified nucleotides at nucleotides 5, 8, 9, 11, 13, 18, and 22 of the guide sequence;
   a shortened repeat/anti-repeat region, wherein nucleotides 38-48 and 53-63 are deleted relative to SEQ ID NO: 3126, comprising:
2′-O-Me modified nucleotides at nucleotides 25, 29, 30, 31, 32, 37, 49-52, 64, 65, 69, 70, and 73;
   a shortened hairpin 1 region, wherein nucleotides 86 and 91 are deleted relative to SEQ ID NO: 3126, comprising:
2′-O-Me modified nucleotides at nucleotides 80, 81, 83, 84, 85, 87-90, 92-94, and 99; 2′-O-Me modified nucleotide at nucleotide 101 between the shortened hairpin 1 region and the shortened hairpin 2 region;
   a shortened hairpin 2 region, wherein nucleotides 112-120 and 127-135 are deleted relative to SEQ ID NO: 3126, comprising:
2′-O-Me modified nucleotides at nucleotides 104, 110, 111, 122-125, 142, and 143, PS linkages between nucleotides 141-142 and 142-143, wherein one or both nucleotides 144-145 are optionally deleted relative to SEQ ID NO: 3126.

In some embodiments, a guide RNA (gRNA) is provided, the gRNA comprising: a guide sequence comprising:
2′-O-Me modified nucleotides at the first four nucleotides 1-4;
PS linkages between nucleotides 1-2, 2-3, and 3-4; and
2′-O-Me modified nucleotides at nucleotides 5, 8, 9, 11, 13, 18, and 22 of the guide sequence;
   a shortened repeat/anti-repeat region, wherein nucleotides 38-48 and 53-63 are deleted relative to SEQ ID NO: 3126, comprising:
2′-O-Me modified nucleotides at nucleotides 25, 29, 30, 31, 32, 37, 49-52, 64, 65, 69, 70, and 73;
   a PS linkage between nucleotides 76-77 between the shortened repeat/anti-repeat region and the shortened hairpin 1 region;
   a shortened hairpin 1 region, wherein nucleotides 86 and 91 are deleted relative to SEQ ID NO: 3126, comprising:
2′-O-Me modified nucleotides at nucleotides 80, 81, 83, 84, 85, 87-90, 92-94, and 99; 2′-O-Me modified nucleotide at nucleotide 101 between the shortened hairpin 1 region and the shortened hairpin 2 region;
   a shortened hairpin 2 region, wherein nucleotides 112-120 and 127-135 are deleted relative to SEQ ID NO: 3126, comprising:
2′-O-Me modified nucleotides at nucleotides 104, 106-111, 122-125, 142, and 143, PS linkages between nucleotides 141-142 and 142-143, wherein one or both nucleotides 144-145 are optionally deleted relative to SEQ ID NO: 3126.

In some embodiments, a guide RNA (gRNA) is provided, the gRNA comprising: a guide sequence comprising:
2′-O-Me modified nucleotides at the first four nucleotides 1-4;
PS linkages between nucleotides 1-2, 2-3, and 3-4; and
2′-O-Me modified nucleotides at nucleotides 5, 8, 9, 11, 13, 18, and 22 of the guide sequence;
   a shortened repeat/anti-repeat region, wherein nucleotides 38-48 and 53-63 are deleted relative to SEQ ID NO: 3126, comprising:
2′-O-Me modified nucleotides at nucleotides 25, 29, 30, 31, 32, 37, 49-52, 64, 65, 69, 70, and 73;
   a shortened hairpin 1 region, wherein nucleotides 86 and 91 are deleted relative to SEQ ID NO: 3126, comprising:
2′-O-Me modified nucleotides at nucleotides 80, 81, 83, 84, 85, 87-90, 92-94, and 99; 2′-O-Me modified nucleotide at nucleotide 101 between the shortened hairpin 1 region and the shortened hairpin 2 region;
   a shortened hairpin 2 region, wherein nucleotides 112-120 and 127-135 are deleted relative to SEQ ID NO: 3126, comprising:
2′-O-Me modified nucleotides at nucleotides 104, 106-111, 122-125, 142, and 143,
PS linkages between nucleotides 141-142 and 142-143,
   wherein one or both nucleotides 144-145 are optionally deleted relative to SEQ ID NO: 3126.

In some embodiments, the guide RNA (gRNA) of the previous embodiment comprising a guide region and a conserved region, the conserved region comprising:
(a) a shortened repeat/anti-repeat region, wherein the shortened repeat/anti-repeat region lacks 18-22 nucleotides, wherein
   (i) nucleotides 37-48 and 53-64 are deleted relative to SEQ ID NO: 3126; and
   (ii) nucleotide 36 is linked to nucleotide 65 by 6-10 nucleotides;
(b) a shortened hairpin 1 region, wherein the shortened hairpin 1 lacks 2 nucleotides relative to SEQ ID NO: 3126, wherein nucleotides 86 and 91 are deleted or nucleotides 85 and 92 are deleted;
(c) a shortened hairpin 2 region, wherein the shortened hairpin 2 lacks 18 nucleotides, wherein nucleotides 113-121 and 126-134 are deleted relative to SEQ ID NO: 3126; and
(d) wherein nucleotides 144-145 are deleted relative to SEQ ID NO: 3126;
wherein at least 10 nucleotides are modified nucleotides.

In further embodiments, the shortened repeat/anti-repeat region, wherein the shortened repeat/anti-repeat region lacks 22 nucleotides relative to SEQ ID NO: 3126. In further embodiments, nucleotide 36 is linked to nucleotide 65 by a sequence comprising the nucleotide sequence UGAAAC. In further embodiments, the nucleotide 36 is linked to nucleotide 65 by 10 nucleotides. In further embodiments, the nucleotide 36 is linked to nucleotide 65 by a sequence comprising the nucleotide sequence UUCGAAAGAC (SEQ ID NO: 3122).

In some embodiments, the sgRNA comprises one of the following sequences in 5′ to 3′ orientation: (N)₂₀₋₂₅ GUUGUAGCUCCCUGAAACCGUUGCUACAAUAAGGCCGUCGAAAGAUGUGCCGC AACGCUCUGCCUUCUGGCAUCGUU (SEQ ID NO: 3119); (N)₂₀₋₂₅ GUUGUAGCUCCCUGAAACCGUUGCUACAAUAAGGCCGUCGAAAGAUGUGCCGC AACGCUCUGCCUUCUGGCAUCGUUUAUU (SEQ ID NO: 3120); (N)₂₀₋₂₅ GUUGUAGCUCCCUGGAAACCCGUUGCUACAAUAAGGCCGUCGAAAGAUGUGCC GCAACGCUCUGCCUUCUGGCAUCGUUUAUU (SEQ ID NO: 3121), where A, C, G, U, and N are adenine, cytosine, guanine, uracil, and any ribonucleotide, respectively, unless otherwise indicated. In some embodiments, N equals 24. In some embodiments, N equals 25.

In some embodiments, at least 10 nucleotides of the conserved portion of the sgRNA are modified nucleotides.

In some embodiments, the sgRNA comprises a conserved region comprising one of the following sequences in 5′ to 3′ orientation:

(SEQ ID NO: 707)
GUUGmUmAmGmCUCCCmUmGmAmAmAmCmCGUUmGmCUAmCAAU*AAGm
GmCCmGmUmCmGmAmAmAmGmAmUGUGCmCGCmAmAmCmGCUCUmGmCC
mUmUmCmUGmGCmAmUC*mG*mU*mU;
or
(SEQ ID NO: 710)
GUUGmUmAmGmCUCCCmUmGmAmAmAmCmCGUUmGmCUAmCAAU*AAGm
GmCCmGmUmCmGmAmAmAmGmAmUGUGCmCGmCAAmCGCUCUmGmCCmU
mUmCmUGGCAUCG*mU*mU;

or any other modified conserved region motifs disclosed in Table 9, including any one of SEQ ID NOs: 715-723, where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides.

In certain embodiments, the HLA-B guide sequence is 20-25 nucleotides in length ((N)₂₀₋₂₅), wherein each nucleotide may be independently modified. In certain embodiments, each of nucleotides 1-3 of the 5′ end of the guide is independently modified. In certain embodiments, each of nucleotides 1-3 of the 5′ end of the guide is independently modified with a 2′-OMe modification. In certain embodiments, each of nucleotides 1-3 of the 5′ end of the guide is independently modified with a phosphorothioate linkage to the adjacent nucleotide residue. In certain embodiments, each of nucleotides 1-3 of the 5′ end of the guide is independently modified with a 2′-OMe modification and a phosphorothioate linkage to the adjacent nucleotide residue.

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20. In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges. In some embodiments, the 2′ hydroxyl group modification can included “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH2CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a gRNA molecule are deoxynucleotides.

In some embodiments, the gRNAs disclosed herein comprise one of the modification patterns disclosed in WO2018/107028 A1, published Jun. 14, 2018 the contents of which are hereby incorporated by reference in their entirety.

The terms “mA,” “mC,” “mU,” or “mG” may be used to denote a nucleotide that has been modified with 2′-O-Me. The terms “TA,” “fC,” “fU,” or “fG” may be used to denote a nucleotide that has been substituted with 2′-F. A “*” may be used to depict a PS modification. The terms A*, C*, U*, or G* may be used to denote a nucleotide that is linked to the next (e.g., 3′) nucleotide with a PS bond. The terms “mA*,” “mC*,” “mU*,” or “mG*” may be used to denote a nucleotide that has been substituted with 2′-O-Me and that is linked to the next (e.g., 3′) nucleotide with a PS bond.

TABLE 8A
Exemplary spyCas9 sgRNA-1 (SEQ ID NO: 600)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B3-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | CC | | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 8B
exemplary NmeCas9 sgRNA (SEQ ID NO: 3126)

| 1-24 | | | | 25 | 26 | 27 | 28 | 29 | 30 | 31 | 32 | 33 | 34 |
|---|---|---|---|---|---|---|---|---|---|---|---|---|---|
| NNNNNNNNNNNNNNN | | | | G | U | U | G | U | A | G | C | U | C |
| NNNNNNNNN | | | | | | | | | | | | | |
| | | | | Lower stem | | | | | | | | | |
| Guide region | | | | Repeat/Anti-Repeat region | | | | | | | | | |
| 35 | 36 | 37 | 38 | 39 | 40 | 41 | 42 | 43 | 44 | 45 | 46 | 47 | 48 |
| C | C | U | U | U | C | U | C | A | U | U | U | C | G |

Upper stem

Repeat/Anti-Repeat region

| 49 | 50 | 51 | 52 | 53 | 54 | 55 | 56 | 57 | 58 | 59 | 60 | 61 | 62 |
|---|---|---|---|---|---|---|---|---|---|---|---|---|---|
| G | A | A | A | C | G | A | A | A | U | G | A | G | A |

| | |
|---|---|
| Loop | Upper stem |

Repeat/Anti-Repeat region

| 63 | 64 | 65 | 66 | 67 | 68 | 69 | 70 | 71 | 72 | 73 | 74 | 75 | 76 |
|---|---|---|---|---|---|---|---|---|---|---|---|---|---|
| A | C | C | G | U | U | G | C | U | A | C | A | A | U |

Lower Stem

Repeat/Anti-Repeat region

| 77 | 78 | 79 | 80 | 81 | 82 | 83 | 84 | 85 | 86 | 87 | 88 | 89 | 90 |
|---|---|---|---|---|---|---|---|---|---|---|---|---|---|
| A | A | G | G | C | C | G | U | C | U | G | A | A | A |
| | Stem | | | | | | | | | Loop | | | |
| | Hairpin 1 | | | | | | | | | | | | |
| 91 | 92 | 93 | 94 | 95 | 96 | 97 | 98 | 99 | 100 | 101 | 102 | 103 | 104 |
| A | G | A | U | G | U | G | C | C | G | C | A | A | C |
| Stem (96: unpaired) | | | | | | | | | | | Lower stem | | |
| Hairpin 1 | | | | | | | | | | | Hairpin 2 | | |
| 105 | 106 | 107 | 108 | 109 | 110 | 111 | 112 | 113 | 114 | 115 | 116 | 117 | 118 |
| G | C | U | C | U | G | C | C | C | C | U | U | A | A |

| | |
|---|---|
| Bulge | Upper Stem |

Hairpin 2

| 119 | 120 | 121 | 122 | 123 | 124 | 125 | 126 | 127 | 128 | 129 | 130 | 131 | 132 |
|---|---|---|---|---|---|---|---|---|---|---|---|---|---|
| A | G | C | U | U | C | U | G | C | U | U | U | A | A |

| | |
|---|---|
| Loop | Upper Stem |

Hairpin 2

| 133 | 134 | 135 | 136 | 137 | 138 | 139 | 140 | 141 | 142 | 143 | 144 | 145 |
|---|---|---|---|---|---|---|---|---|---|---|---|---|
| G | G | G | G | C | A | U | C | G | U | U | U | A |

| | | |
|---|---|---|
| Upper Stem | Bulge | Lower Stem |

Hairpin 2

### I. Ribonucleoprotein Complex

In some embodiments, the disclosure provides compositions comprising one or more gRNAs comprising one or more guide sequences from Tables 2-7 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, N. meningitidis, S. aureus, and other prokaryotes (see e.g., the list in the next paragraph), and modified (e.g., engineered or mutant) versions thereof. See e.g., US2016/0312198 A1; US 2016/0312199 A1. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015). In some embodiments, the RNA-guided DNA-binding agent comprises a Cas nickase. In some embodiments, the RNA-guided nickase is modified or derived from a Cas protein, such as a Class 2 Cas nuclease (which may be, e.g., a Cas nuclease of Type II, V, or VI). Class 2 Cas nuclease include, for example, Cas9, Cpf1, C2c1, C2c2, and C2c3 proteins and modifications thereof.

Non-limiting exemplary species that the Cas nuclease or Cas nickase can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the Cas nickase is derived from the Cas9 nuclease from Streptococcus pyogenes. In some embodiments, the Cas nickase is derived from the Cas9 nuclease from Streptococcus thermophilus. In some embodiments, the Cas nickase is a nickase form of the Cas9 nuclease from Neisseria meningitidis. See e.g., WO/2020081568, describing an Nme2Cas9 D16A nickase fusion protein. In some embodiments, the Cas nickase is derived from the Cas9 nuclease is from Staphylococcus aureus. In some embodiments, the Cas nickase is derived from the Cpf1 nuclease from Francisella novicida. In some embodiments, the Cas nickase is derived from the Cpf1 nuclease from Acidaminococcus sp. In some embodiments, the Cas nickase is derived from the Cpf1 nuclease from Lachnospiraceae bacterium ND2006. In further embodiments, the Cas nickase is derived from the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nickase is derived from a Cpf1 nuclease from an Acidaminococcus or Lachnospiraceae. As discussed elsewhere, a nickase may be derived from a nuclease by inactivating one of the two catalytic domains, e.g., by mutating an active site residue essential for nucleolysis, such as D10, H840, of N863 in Spy Cas9. One skilled in the art will be familiar with techniques for easily identifying corresponding residues in other Cas proteins, such as sequence alignment and structural alignment, which is discussed in detail below.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease or Cas nickase may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein. See, e.g., Zetsche et al. (2015). Exemplary amino acid substitutions in the HNH or HNH-like nuclease domain or RuvC or RuvC-like domains for N. meningitidis include Nme2Cas9D16A (HNH nickase) and Nme2Cas9H588A (RuvC nickase). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA binding agent comprises one or more heterologous functional domains (e.g., is or comprises a fusion polypeptide).

In some embodiments, the RNA-guided DNA binding agent comprises a APOBEC3 deaminase. In some embodiments, a APOBEC3 deaminase is a APOBEC3A (A3A). In some embodiments, the A3A is a human A3A. In some embodiments, the A3A is a wild-type A3A.

In some embodiments, the RNA-guided DNA binding agent comprises a deaminase and an RNA-guided nickase. In some embodiments, the mRNA further comprises a linker to link the sequencing encoding A3A to the sequence sequencing encoding RNA-guided nickase. In some embodiments, the linker is an organic molecule, group, polymer, or chemical moiety. In some embodiments, the linker is a peptide linker. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 900), SGSETPGTSESA (SEQ ID NO: 901), or SGSETPGTSESATPEGGSGGS (SEQ ID NO: 902).

In some embodiments, the peptide linker comprises a (GGGGS)ₙ (SEQ ID NO: 910), a (G)ₙ, an (EAAAK)ₙ (SEQ ID NO: 911), a (GGS)ₙ, an SGSETPGTSESATPES (SEQ ID NO: 906) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif, or a combination of any of these, wherein n is independently an integer between 1 and 30 (SEQ DI NO: 3123). See, WO2015089406, e.g., paragraph [0012], the entire content of which is incorporated herein by reference.

In some embodiments, the peptide linker comprises one or more sequences selected from SEQ ID NOs: 906-970.

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be fused at the N-terminus or the C-terminus of the RNA-guided DNA-binding agent sequence. In some embodiments, the NLS is not fused to the C-terminus. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two NLS sequences (e.g., SV40) fused at the carboxy terminus. In some embodiments, the RNA-guided DNA-binding agent may be fused with two NLSs, one at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 903) or PKKKRRV (SEQ ID NO: 904). In some embodiments, the NLS may be a bipartite sequence, such as the NLS of nucleoplasmin, KRPAATKKAGQAKKKK (SEQ ID NO: 905). In a specific embodiment, a single PKKKRKV (SEQ ID NO: 903) NLS may be fused at the C-terminus of the RNA-guided DNA-binding agent. One or more linkers are optionally included at the fusion site.

In some embodiments, the RNA-guided DNA binding agent comprises an editor. An exemplary editor is BC22n which includes a H. sapiens APOBEC3A fused to S. pyogenes-DIOA Cas9 nickase by an XTEN linker, and mRNA encoding BC22n. An mRNA encoding BC22n is provided (SEQ ID NO: 804).

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUBI), membrane-anchored UBL (MUB), ubiquitin fold-modifier-1 (UFMI), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His (SEQ ID NO: 3124), 8×His (SEQ ID NO: 3125), biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain such as an editor domain. When the RNA-guided DNA-binding agent is directed to its target sequence, e.g., when a Cas nuclease is directed to a target sequence by a gRNA, the effector such as an editor domain may modify or affect the target sequence. In some embodiments, the effector such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

### J. Determination of Efficacy of Guide RNAs

In some embodiments, the efficacy of a guide RNA is determined when delivered or expressed together with other components (e.g., an RNA-guided DNA binding agent) forming an RNP. In some embodiments, the guide RNA is expressed together with an RNA-guided DNA binding agent, such as a Cas protein, e.g., Cas9. In some embodiments, the guide RNA is delivered to or expressed in a cell line that already stably expresses an RNA-guided DNA nuclease, such as a Cas nuclease or nickase, e.g., Cas9 nuclease or nickase. In some embodiments the guide RNA is delivered to a cell as part of a RNP. In some embodiments, the guide RNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and a guide RNA disclosed herein can lead to DSBs, SSBs, or site-specific binding that results in nucleic acid modification in the DNA or pre-mRNA which can produce errors in the form of insertion/deletion (indel) mutations upon repair by cellular machinery. Many mutations due to indels alter the reading frame, introduce premature stop codons, or induce exon skipping and, therefore, produce a non-functional protein.

In some embodiments, the efficacy of particular guide RNAs is determined based on in vitro models. In some embodiments, the in vitro model is T cell line. In some embodiments, the in vitro model is HEK293 T cells. In some embodiments, the in vitro model is HEK293 cells stably expressing Cas9 (HEK293_Cas9). In some embodiments, the in vitro model is a lymphoblastoid cell line. In some embodiments, the in vitro model is primary human T cells. In some embodiments, the in vitro model is primary human B cells. In some embodiments, the in vitro model is primary human peripheral blood lymphocytes. In some embodiments, the in vitro model is primary human peripheral blood mononuclear cells.

In some embodiments, the number of off-target sites at which a deletion or insertion occurs in an in vitro model is determined, e.g., by analyzing genomic DNA from the cells transfected in vitro with Cas9 mRNA and the guide RNA. In some embodiments, such a determination comprises analyzing genomic DNA from cells transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of a guide RNA is evaluated by on target cleavage efficiency. In some embodiments, the efficacy of a guide RNA is measured by percent editing at the target location, e.g., HLA-A, HLA-B, or CIITA. In some embodiments, deep sequencing may be utilized to identify the presence of modifications (e.g., insertions, deletions) introduced by gene editing. Indel percentage can be calculated from next generation sequencing “NGS.”

In some embodiments, the efficacy of a guide RNA is measured by the number or frequency of indels at off-target sequences within the genome of the target cell type. In some embodiments, efficacious guide RNAs are provided which produce indels at off target sites at very low frequencies (e.g., <5%) in a cell population or relative to the frequency of indel creation at the target site. Thus, the disclosure provides for guide RNAs which do not exhibit off-target indel formation in the target cell type (e.g., T cells or B cells), or which produce a frequency of off-target indel formation of <5% in a cell population or relative to the frequency of indel creation at the target site. In some embodiments, the disclosure provides guide RNAs which do not exhibit any off target indel formation in the target cell type (e.g., T cells or B cells). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., T cells or B cells) genome.

In some embodiments, linear amplification is used to detect gene editing events, such as the formation of insertion/deletion (“indel”) mutations, translocations, and homology directed repair (HDR) events in target DNA. For example, linear amplification with a unique sequence-tagged primer and isolating the tagged amplification products (herein after referred to as “UnIT,” or “Unique Identifier Tagmentation” method) may be used.

In some embodiments, the efficacy of a guide RNA is measured by the number of chromosomal rearrangements within the target cell type. Kromatid dGH assay may used to detect chromosomal rearrangements, including e.g., translocations, reciprocal translocations, translocations to off-target chromosomes, deletions (i.e., chromosomal rearrangements where fragments were lost during the cell replication cycle due to the editing event). In some embodiments, the target cell type has less than 10, less than 8, less than 5, less than 4, less than 3, less than 2, or less than 1 chromosomal rearrangement. In some embodiments, the target cell type has no chromosomal rearrangements.

### K. Delivery of gRNA Compositions

Lipid nanoparticles (LNP compositions) are a well-known means for delivery of nucleotide and protein cargo and may be used for delivery of the guide RNAs, compositions, or pharmaceutical formulations disclosed herein. In some embodiments, the LNP compositions deliver nucleic acid, protein, or nucleic acid together with protein.

In some embodiments, the invention comprises a method for delivering any one of the gRNAs disclosed herein to a subject, wherein the gRNA is formulated as an LNP. In some embodiments, the LNP comprises the gRNA and a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNP compositions comprise cationic lipids. In some embodiments, the LNP compositions comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) (Lipid A) or another ionizable lipid. See, e.g., lipids of WO/2017/173054 and references described therein. In some embodiments, the LNP compositions comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, the LNP comprises a lipid component, and the lipid component comprises: about 35 mol % Lipid A; about 15 mol % neutral lipid (e.g., distearoylphosphatidylcholine (DSPC)); about 47.5 mol % helper lipid (e.g., cholesterol); and about 2.5 mol % stealth lipid (e.g., 1,2-dimyristoyl-rac-gly cero-3-methylpolyoxyethylene glycol 2000 (PEG2k-DMG)), and wherein the N/P ratio of the LNP composition is about 3-7.

In some embodiments, the gRNAs disclosed herein are formulated as LNP compositions for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is formulated as an LNP or not formulated as an LNP. In some embodiments, the LNP comprises the gRNA and a Cas9 or an mRNA encoding Cas9.

In some embodiments, the guide RNA compositions described herein, alone or encoded on one or more vectors, are formulated in or administered via a lipid nanoparticle; see e.g., WO/2017/173054 and WO 2019/067992, the contents of which are hereby incorporated by reference in their entirety.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from S. pyogenes (i.e., Spy Cas9). In one embodiment, the Cas9 nuclease is from N. meningitidis (i.e., Nine Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

### L. Therapeutic Methods and Uses

Any of the engineered human cells and compositions described herein can be used in a method of treating a variety of diseases and disorders, as described herein. In some embodiments, the genetically modified cell (engineered cell) or population of genetically modified cells (engineered cells) and compositions may be used in methods of treating a variety of diseases and disorders. In some embodiments, a method of treating any one of the diseases or disorders described herein is encompassed, comprising administering any one or more composition described herein.

In some embodiments, the methods and compositions described herein may be used to treat diseases or disorders in need of delivery of a therapeutic agent. In some embodiments, the invention provides a method of providing an immunotherapy in a subject, the method including administering to the subject an effective amount of an engineered cell (or population of engineered cells) as described herein, for example, a cell of any of the aforementioned cell aspects and embodiments.

In some embodiments, the methods comprise administering to a subject a composition comprising an engineered cell described herein as an adoptive cell transfer therapy. In some embodiments, the engineered cell is an allogeneic cell.

In some embodiments, the methods comprise administering to a subject a composition comprising an engineered cell described herein, wherein the cell produces, secretes, or expresses a polypeptide (e.g., a targeting receptor) useful for treatment of a disease or disorder in a subject. In some embodiments, the cell acts as a cell factory to produce a soluble polypeptide. In some embodiments, the cell acts as a cell factory to produce an antibody. In some embodiments, the cell continuously secretes the polypeptide in vivo. In some embodiments, the cell continuously secretes the polypeptide following transplantation in vivo for at least 1, 2, 3, 4, 5, or 6 weeks. In some embodiments, the cell continuously secretes the polypeptide following transplantation in vivo for more than 6 weeks. In some embodiments, the soluble polypeptide (e.g., an antibody) is produced by the cell at a concentration of at least 10², 10³, 104, 105, 10⁶, 107, or 10⁸ copies per day. In some embodiments, the polypeptide is an antibody and is produced by the cell at a concentration of at least 10⁸ copies per day.

In some embodiments of the methods, the method includes administering a lymphodepleting agent or immunosuppressant prior to administering to the subject an effective amount of the engineered cell (or engineered cells) as described herein, for example, a cell of any of the aforementioned cell aspects and embodiments. In another aspect, the invention provides a method of preparing engineered cells (e.g., a population of engineered cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer (NK) cells, cytotoxic T lymphocytes (CTLs), T helper cells, B cells, or their progenitors such as hematopoietic stem cells (HSC) or induced pluripotent stem cells (iPSC) can be programmed to act in response to abnormal antigens expressed on the surface of tumor cells. Thus, cancer immunotherapy allows components of the immune system to destroy tumors or other cancerous cells. Cell-based immunotherapies have also been demonstrated to be effective in the treatment of autoimmune diseases or transplant rejection. Immune effector cells such as regulatory T cells (Tregs) or mesenchymal stem cells can be programmed to act in response to autoantigens or transplant antigens expressed on the surface of normal tissues.

In some embodiments, the invention provides a method of preparing engineered cells (e.g., a population of engineered cells). The population of engineered cells may be used for immunotherapy.

In some embodiments, the invention provides a method of treating a subject in need thereof that includes administering engineered cells prepared by a method of preparing cells described herein, for example, a method of any of the aforementioned aspects and embodiments of methods of preparing cells.

In some embodiments, the engineered cells can be used to treat cancer, infectious diseases, inflammatory diseases, autoimmune diseases, cardiovascular diseases, neurological diseases, ophthalmologic diseases, renal diseases, liver diseases, musculoskeletal diseases, red blood cell diseases, or transplant rejections. In some embodiments, the engineered cells can be used in cell transplant, e.g., to the heart, liver, lung, kidney, pancreas, skin, or brain. (See e.g., Deuse et al., Nature Biotechnology 37:252-258 (2019).)

In some embodiments, the engineered cells can be used as a cell therapy comprising an allogeneic stem cell therapy. In some embodiments, the cell therapy comprises induced pluripotent stem cells (iPSCs). iPSCs may be induced to differentiate into other cell types including e.g., cardiomyocytes, beta islet cells, neurons, and blood cells. In some embodiments, the cell therapy comprises hematopoietic stem cells. In some embodiments, the stem cells comprise mesenchymal stem cells that can develop into bone, cartilage, muscle, and fat cells. In some embodiments, the stem cells comprise ocular stem cells. In some embodiments, the allogeneic stem cell transplant comprises allogeneic bone marrow transplant. In some embodiments, the stem cells comprise pluripotent stem cells (PSCs). In some embodiments, the stem cells comprise induced embryonic stem cells (ESCs).

The engineered human cells disclosed herein are suitable for further engineering, e.g., by introduction of further edited, or modified genes or alleles. Cells of the invention may also be suitable for further engineering by introduction of an exogenous nucleic acid encoding e.g., a targeting receptor, e.g., a TCR, CAR, UniCAR. CARs are also known as chimeric immunoreceptors, chimeric T cell receptors or artificial T cell receptors. In some embodiments, the TCR is a wild-type or variant TCR.

In some embodiments, the cell therapy is a transgenic T cell therapy. In some embodiments, the cell therapy comprises a Wilms' Tumor 1 (WT1) targeting transgenic T cell. In some embodiments, the cell therapy comprises a targeting receptor or a donor nucleic acid encoding a targeting receptor of a commercially available T cell therapy, such as a CAR T cell therapy. There are number of targeting receptors currently approved for cell therapy. The cells and methods provided herein can be used with these known constructs. Commercially approved cell products that include targeting receptor constructs for use as cell therapies include e.g., Kymriah® (tisagenlecleucel); Yescarta® (axicabtagene ciloleucel); Tecartus™ (brexucabtagene autoleucel); Tabelecleucel (Tab-cel®); Viralym-M (ALVR105); and Viralym-C.

In some embodiments, the methods provide for administering the engineered cells to a subject, wherein the administration is an injection. In some embodiments, the methods provide for administering the engineered cells to a subject, wherein the administration is an intravascular injection or infusion. In some embodiments, the methods provide for administering the engineered cells to a subject, wherein the administration is a single dose.

In some embodiments, the methods provide for reducing a sign or symptom associated of a subject's disease treated with a composition disclosed herein. In some embodiments, the subject has a response to treatment with a composition disclosed herein that lasts more than one week. In some embodiments, the subject has a response to treatment with a composition disclosed herein that lasts more than two weeks. In some embodiments, the subject has a response to treatment with a composition disclosed herein that lasts more than three weeks. In some embodiments, the subject has a response to treatment with a composition disclosed herein that lasts more than one month.

In some embodiments, the methods provide for administering the engineered cells to a subject, and wherein the subject has a response to the administered cell that comprises a reduction in a sign or symptom associated with the disease treated by the cell therapy. In some embodiments, the subject has a response that lasts more than one week. In some embodiments, the subject has a response that lasts more than one month. In some embodiments, the subject has a response that lasts for at least 1-6 weeks.

TABLE 9
VI. ADDITIONAL SEQUENCES
*The guide sequence disclosed in this Table may be unmodified, modified with the exemplary modification pattern shown in the Table, or
modified with a different modification pattern disclosed herein or available in the art.

| | SEQ ID | |
|---|---|---|
| Description | NO | Sequence |
| Exemplary SpyCas9 | 600 | GUUUUAGAGCUAGAAAUAGCAAGUUAAAAUAAGGCUAGUCCGUUAUCAACUUGAAAAAGUGGCACCGAGUCGGUGC |
| sgRNA scaffold | | |
| Exemplary SpyCas 9 | 601 | GUUUUAGAGCUAUGCUGUUUUG |
| crRNA 3′ Sequence | | |
| Exemplary SpyCas 9 | 602 | GUUUUAGAGCUAGAAAUAGCAAGUUAAAAUAAGGCUAGUCCGUUAUCAACUUGAAAAAGUGGCACCGAGUCGGUGCUUUU |
| sgRNA Scaffold | | |
| Exemplary SpyCas9 | 603 | GUUUUAGAGCUAGAAAUAGCAAGUUAAAAUAAGGCUAGUCCGUUAUCAACUUGAAAAAGUGGCACCGAGUCGGUGC |
| sgRNA Scaffold | | |
| Exemplary SpyCas9 | 604 | GUUUUAGAGCUAGAAAUAGCAAGUUAAAAUAAGGCUAGUCCGUUAUCAACUUGGCACCGAGUCGGUGC |
| crRNA Scaffold | | |
| | 605-612 | See Table 9A |
| | 613-698 | Not used |
| Exemplary NmeCas9 | 699 | GUUGUAGCUCCCUUUCUCAUUUCGGAAACGAAAUGAGAACCGUUGCUACAAUAAGGCCGUCUGAAAAGAUGUGCCGCAACGCUCU |
| guide RNA scaffold | | GCCCCUUAAAGCUUCUGCUUUAAGGGGCAUCGUUUA |
| Exemplary NmeCas 9 | 700 | (N)₂₀₋₂₅ |
| guide RNA | | GUUGUAGCUCCCUUUCUCAUUUCGGAAACGAAAUGAGAACCGUUGCUACAAUAAGGCCGUCUGAAAAGAUGUGCCGCAACGCUCU |
| | | GCCCCUUAAAGCUUCUGCUUUAAGGGGCAUCGUUUA |
| Shortened/unmodified | 3119 | (N)₂₀₋₂₅GUUGUAGCUCCCUGAAACCGUUGCUACAAUAAGGCCGUCGAAAGAUGUGCCGCAACGCUCUGCCUUCUGGCAUCGUU |
| NmeCas9 guide RNA | | |
| motif | | |
| Shortened/unmodified | 3120 | (N)₂₀₋₂₅ |
| NmeCas9 guide RNA | | GUUGUAGCUCCCUGAAACCGUUGCUACAAUAAGGCCGUCGAAAGAUGUGCCGCAACGCUCUGCCUUCUGGCAUCGUUUAUU |
| motif | | |
| Shortened/unmodified | 3121 | (N)₂₀₋₂₅ |
| NmeCas9 guide RNA | | GUUGUAGCUCCCUGGAAACCCGUUGCUACAAUAAGGCCGUCGAAAGAUGUGCCGCAACGCUCUGCCUUCUGGCAUCGUUUAUU |
| motif | | |
| Shortened/unmodified | 704 | (N)₂₀₋₂₅ |
| NmeCas9 guide RNA | | GUUGUAGCUCCCUUCGAAAGACCGUUGCUACAAUAAGGCCGUCGAAAGAUGUGCCGCAACGCUCUGCCUUCUGGCAUCGUU |
| motif | | |
| Shortened/modified | 705 | mN*mNNNNNNNNmNNNmNNNNNNNNNNNNmGUUGmUmAmGmCUCCCmUmGmAmAmAmCmCGUUmGmCUAmCAAU*AAGmGmCCmG |
| NmeCas9 guide RNA | | mUmCmGmAmAmAmGmAmUGUGCmCGCmAmAmCmGCUCUmGmCCmUmUmCmUGmGCmAmUC*mG*mU*mU |
| motif (101-mer) | | |
| Shortened/modified | 706 | (N)₂₀₋₂₅GUUGmUmAmGmCUCCCmUmGmAmAmAmCmCGUUmGmCUAmCAAU*AAGmGmCCmGmUmCmGm |
| NmeCas9 guide RNA | | AmAmAmGmAmUGUGCmCGCmAmAmCmGCUCUmGmCCmUmUmCmUGmGCmAmUC*mG*mU*mU |
| motif | | |
| Shortened/modified | 707 | GUUGmUmAmGmCUCCCmUmGmAmAmAmCmCGUUmGmCUAmCAAU*AAGmGmCCmGmUmCmGmAmAmAmGmAmUGUGCmCGCmAmA |
| NmeCas9 guide RNA | | mCmGCUCUmGmCCmUmUmCmUGmGCmAmUC*mG*mU*mU |
| motif | | |
| Shortened/modified | 708 | mN*mN*mN*mNmNNNmNmNNmNNmNNNNNmNNNNmNNNmGUUGmUmAmGmCUCCCmUmGmAmAmAmCmCGUUmGmCUAmCAAU*A |
| NmeCas9 guide RNA | | AGmGmCCmGmUmCmGmAmAmAmGmAmUGUGCmCGmCAAmCGCUCUmGmCCmUmUmCmUGGCAUCG*mU*mU |
| motif (101-mer) | | |
| Shortened/modified | 709 | (N)₂₀₋₂₅GUUGmUmAmGmCUCCCmUmGmAmAmAmCmCGUUmGmCUAmCAAU*AAGmGmCCmGmUmCmGm |
| NmeCas9 guide RNA | | AmAmAmGmAmUGUGCmCGmCAAmCGCUCUmGmCCmUmUmCmUGGCAUCG*mU*mU |
| motif | | |
| Shortened/modified | 710 | GUUGmUmAmGmCUCCCmUmGmAmAmAmCmCGUUmGmCUAmCAAU*AAGmGmCCmGmUmCmGmAmAmAmGmAmUGUGCmCGmCAAm |
| NmeCas9 guide RNA | | CGCUCUmGmCCmUmUmCmUGGCAUCG*mU*mU |
| motif | | |
| Shortened/modified | 711 | mN*mN*mN*mNmNNNmNmNNmNNmNNNNNmNNNNmNNNmGUUGmUmAmGmCUCCCmUmUmCmGmAmAmAmGmAmCmCGUUmGmCU |
| NmeCas9 guide RNA | | AmCAAU*AAGmGmCCmGmUmCmGmAmAmAmGmAmUGUGCmCGmCAAmCGCUCUmGmCCmUmUmCmUGGCAUCG*mU*mU |
| motif (105-mer) | | |
| Shortened/modified | 712 | mN*mN*mN*mNmNNNmNmNNmNNmNNNNNmNNNNmNNNmGUUGmUmAmGmCUCCCmUmGmAmAmAmCmCGUUmGmCUAmCAAUAA |
| NmeCas9 guide RNA | | GmGmCCmGmUmCmGmAmAmAmGmAmUGUGCmCGmCAAmCGCUCUmGmCCmUmUmCmUGGCAUCG*mU*mU |
| motif (101-mer) | | |
| Shortened/modified | 713 | mN*mN*mN*mNmNNNmNmNNmNNmNNNNNmNNNNmNNNmGUUGmUmAmGmCUCCCmUmGmAmAmAmCmCGUUmGmCUAmCAAU*A |
| NmeCas9 guide RNA | | AGmGmCCmGmUmCmGmAmAmAmGmAmUGUGCmCGmCAAmCGmCmUmCmUmGmCCmUmUmCmUGGCAUCG*mU*mU |
| motif (101-mer) | | |
| Shortened/modified | 714 | mN*mN*mN*mNmNNNmNmNNmNNmNNNNNmNNNNmNNNmGUUGmUmAmGmCUCCCmUmGmAmAmAmCmCGUUmGmCUAmCAAUAA |
| NmeCas9 guide RNA | | GmGmCCmGmUmCmGmAmAmAmGmAmUGUGCmCGmCAAmCGmCmUmCmUmGmCCmUmUmCmUGGCAUCG*mU*mU |
| motif (101-mer) | | |
| Guide scaffold | 715 | mGUUGmUmAmGmCUCCCmUmGmAmAmAmCmCGUUmGmCUAmCAAU*AAGmGmCCmGmUmCmGmAmAmAmGmAmUGUGCmCGCmAm |
| | | AmCmGCUCUmGmCCmUmUmCmUGmGCmAmUC*mG*mU*mU |
| Guide scaffold | 716 | mGUUGmUmAmGmCUCCCmUmGmAmAmAmCmCGUUmGmCUAmCAAU*AAGmGmCCmGmUmCmGmAmAmAmGmAmUGUGCmCGmCAA |
| | | mCGCUCUmGmCCmUmUmCmUGGCAUCG*mU*mU |
| Guide scaffold | 717 | mGUUGmUmAmGmCUCCCmUmGmAmAmAmCmCGUUmGmCUAmCAAUAAGmGmCCmGmUmCmGmAmAmAmGmAmUGUGCmCGmCAAm |
| | | CGCUCUmGmCCmUmUmCmUGGCAUCG*mU*mU |
| Guide scaffold | 718 | mGUUGmUmAmGmCUCCCmUmGmAmAmAmCmCGUUmGmCUAmCAAU*AAGmGmCCmGmUmCmGmAmAmAmGmAmUGUGCmCGmCAA |
| | | mCGmCmUmCmUmGmCCmUmUmCmUGGCAUCG*mU*mU |
| Guide scaffold | 719 | mGUUGmUmAmGmCUCCCmUmGmAmAmAmCmCGUUmGmCUAmCAAUAAGmGmCCmGmUmCmGmAmAmAmGmAmUGUGCmCGmCAAm |
| | | CGmCmUmCmUmGmCCmUmUmCmUGGCAUCG*mU*mU |
| Guide scaffold | 720 | mGUUGmUmAmGmCUCCCmUmUmCmGmAmAmAmGmAmCmCGUUmGmCUAmCAAU*AAGmGmCCmGmUmCmGmAmAmAmGmAmUGUG |
| | | CmCGmCAAmCGCUCUmGmCCmUmUmCmUGGCAUCG*mU*mU |
| Guide scaffold | 721 | mGUUGmUmAmGmCUCCCmUmUmCmGmAmAmAmGmAmCmCGUUmGmCUAmCAAUAAGmGmCCmGmUmCmGmAmAmAmGmAmUGUGC |
| | | mCGmCAAmCGCUCUmGmCCmUmUmCmUGGCAUCG*mU*mU |
| Guide scaffold | 722 | mGUUGmUmAmGmCUCCCmUmUmCmGmAmAmAmGmAmCmCGUUmGmCUAmCAAU*AAGmGmCCmGmUmCmGmAmAmAmGmAmUGUG |
| | | CmCGmCAAmCGmCmUmCmUmGmCCmUmUmCmUGGCAUCG*mU*mU |
| Guide scaffold | 723 | mGUUGmUmAmGmCUCCCmUmUmCmGmAmAmAmGmAmCmCGUUmGmCUAmCAAUAAGmGmCCmGmUmCmGmAmAmAmGmAmUGUGC |
| | | mCGmCAAmCGmCmUmCmUmGmCCmUmUmCmUGGCAUCG*mU*mU |
| | 724-799 | Not used |
| Recombinant Cas9-NLS | 800 | MDKKYSIGLDIGTNSVGWAVITDEYKVPSKKFKVLGNTDRHSIKKNLIGALLFDSGETAEATRLKRTARRRYTRRKNRICYLQEI |
| amino acid sequence | | FSNEMAKVDDSFFHRLEESFLVEEDKKHERHPIFGNIVDEVAYHEKYPTIYHLRKKLVDSTDKADLRLIYLALAHMIKFRGHFLI |
| | | EGDLNPDNSDVDKLFIQLVQTYNQLFEENPINASGVDAKAILSARLSKSRRLENLIAQLPGEKKNGLFGNLIALSLGLTPNFKSN |
| | | FDLAEDAKLQLSKDTYDDDLDNLLAQIGDQYADLFLAAKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEELLVKLNREDLLRKQRTFDNGSIPHQIHLGELHAILRR |
| | | QEDFYPFLKDNREKIEKILTFRIPYYVGPLARGNSRFAWMTRKSEETITPWNFEEVVDKGASAQSFIERMTNFDKNLPNEKVLPK |
| | | HSLLYEYFTVYNELTKVKYVTEGMRKPAFLSGEQKKAIVDLLFKTNRKVTVKQLKEDYFKKIECFDSVEISGVEDRFNASLGTYH |
| | | DLLKIIKDKDFLDNEENEDILEDIVLTLTLFEDREMIEERLKTYAHLEDDKVMKQLKRRRYTGWGRLSRKLINGIRDKQSGKTIL |
| | | DFLKSDGFANRNFMQLIHDDSLTFKEDIQKAQVSGQGDSLHEHIANLAGSPAIKKGILQTVKVVDELVKVMGRHKPENIVIEMAR |
| | | ENQTTQKGQKNSRERMKRIEEGIKELGSQILKEHPVENTQLQNEKLYLYYLQNGRDMYVDQELDINRLSDYDVDHIVPQSELKDD |
| | | SIDNKVLTRSDKNRGKSDNVPSEEVVKKMKNYWRQLLNAKLITQRKFDNLTKAERGGLSELDKAGFIKRQLVETRQITKHVAQIL |
| | | DSRMNTKYDENDKLIREVKVITLKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIMERSSFEKNPIDFLEAKGYKEV |
| | | KKDLIIKLPKYSLFELENGRKRMLASAGELQKGNELALPSKYVNFLYLASHYEKLKGSPEDNEQKQLFVEQHKHYLDEIIEQISE |
| | | FSKRVILADANLDKVLSAYNKHRDKPIREQAENIIHLFTLTNLGAPAAFKYFDTTIDRKRYTSTKEVLDATLIHQSITGLYETRI |
| | | DLSQLGGDGGGSPKKKRKV |
| ORF encoding Spy Cas9 | 801 | ATGGACAAGAAGTACAGCATCGGACTGGACATCGGAACAAACAGCGTCGGATGGGCAGTCATCACAGACGAATACAAGGTCCCGA |
| | | GCAAGAAGTTCAAGGTCCTGGGAAACACAGACAGACACAGCATCAAGAAGAACCTGATCGGAGCACTGCTGTTCGACAGCGGAGA |
| | | AACAGCAGAAGCAACAAGACTGAAGAGAACAGCAAGAAGAAGATACACAAGAAGAAAGAACAGAATCTGCTACCTGCAGGAAATC |
| | | TTCAGCAACGAAATGGCAAAGGTCGACGACAGCTTCTTCCACAGACTGGAAGAAAGCTTCCTGGTCGAAGAAGACAAGAAGCACG |
| | | AAAGACACCCGATCTTCGGAAACATCGTCGACGAAGTCGCATACCACGAAAAGTACCCGACAATCTACCACCTGAGAAAGAAGCT |
| | | GGTCGACAGCACAGACAAGGCAGACCTGAGACTGATCTACCTGGCACTGGCACACATGATCAAGTTCAGAGGACACTTCCTGATC |
| | | GAAGGAGACCTGAACCCGGACAACAGCGACGTCGACAAGCTGTTCATCCAGCTGGTCCAGACATACAACCAGCTGTTCGAAGAAA |
| | | ACCCGATCAACGCAAGCGGAGTCGACGCAAAGGCAATCCTGAGCGCAAGACTGAGCAAGAGCAGAAGACTGGAAAACCTGATCGC |
| | | ACAGCTGCCGGGAGAAAAGAAGAACGGACTGTTCGGAAACCTGATCGCACTGAGCCTGGGACTGACACCGAACTTCAAGAGCAAC |
| | | TTCGACCTGGCAGAAGACGCAAAGCTGCAGCTGAGCAAGGACACATACGACGACGACCTGGACAACCTGCTGGCACAGATCGGAG |
| | | ACCAGTACGCAGACCTGTTCCTGGCAGCAAAGAACCTGAGCGACGCAATCCTGCTGAGCGACATCCTGAGAGTCAACACAGAAAT |
| | | CACAAAGGCACCGCTGAGCGCAAGCATGATCAAGAGATACGACGAACACCACCAGGACCTGACACTGCTGAAGGCACTGGTCAGA |
| | | CAGCAGCTGCCGGAAAAGTACAAGGAAATCTTCTTCGACCAGAGCAAGAACGGATACGCAGGATACATCGACGGAGGAGCAAGCC |
| | | AGGAAGAATTCTACAAGTTCATCAAGCCGATCCTGGAAAAGATGGACGGAACAGAAGAACTGCTGGTCAAGCTGAACAGAGAAGA |
| | | CCTGCTGAGAAAGCAGAGAACATTCGACAACGGAAGCATCCCGCACCAGATCCACCTGGGAGAACTGCACGCAATCCTGAGAAGA |
| | | CAGGAAGACTTCTACCCGTTCCTGAAGGACAACAGAGAAAAGATCGAAAAGATCCTGACATTCAGAATCCCGTACTACGTCGGAC |
| | | CGCTGGCAAGAGGAAACAGCAGATTCGCATGGATGACAAGAAAGAGCGAAGAAACAATCACACCGTGGAACTTCGAAGAAGTCGT |
| | | CGACAAGGGAGCAAGCGCACAGAGCTTCATCGAAAGAATGACAAACTTCGACAAGAACCTGCCGAACGAAAAGGTCCTGCCGAAG |
| | | CACAGCCTGCTGTACGAATACTTCACAGTCTACAACGAACTGACAAAGGTCAAGTACGTCACAGAAGGAATGAGAAAGCCGGCAT |
| | | TCCTGAGCGGAGAACAGAAGAAGGCAATCGTCGACCTGCTGTTCAAGACAAACAGAAAGGTCACAGTCAAGCAGCTGAAGGAAGA |
| | | CTACTTCAAGAAGATCGAATGCTTCGACAGCGTCGAAATCAGCGGAGTCGAAGACAGATTCAACGCAAGCCTGGGAACATACCAC |
| | | GACCTGCTGAAGATCATCAAGGACAAGGACTTCCTGGACAACGAAGAAAACGAAGACATCCTGGAAGACATCGTCCTGACACTGA |
| | | CACTGTTCGAAGACAGAGAAATGATCGAAGAAAGACTGAAGACATACGCACACCTGTTCGACGACAAGGTCATGAAGCAGCTGAA |
| | | GAGAAGAAGATACACAGGATGGGGAAGACTGAGCAGAAAGCTGATCAACGGAATCAGAGACAAGCAGAGCGGAAAGACAATCCTG |
| | | GACTTCCTGAAGAGCGACGGATTCGCAAACAGAAACTTCATGCAGCTGATCCACGACGACAGCCTGACATTCAAGGAAGACATCC |
| | | AGAAGGCACAGGTCAGCGGACAGGGAGACAGCCTGCACGAACACATCGCAAACCTGGCAGGAAGCCCGGCAATCAAGAAGGGAAT |
| | | CCTGCAGACAGTCAAGGTCGTCGACGAACTGGTCAAGGTCATGGGAAGACACAAGCCGGAAAACATCGTCATCGAAATGGCAAGA |
| | | GAAAACCAGACAACACAGAAGGGACAGAAGAACAGCAGAGAAAGAATGAAGAGAATCGAAGAAGGAATCAAGGAACTGGGAAGCC |
| | | AGATCCTGAAGGAACACCCGGTCGAAAACACACAGCTGCAGAACGAAAAGCTGTACCTGTACTACCTGCAGAACGGAAGAGACAT |
| | | GTACGTCGACCAGGAACTGGACATCAACAGACTGAGCGACTACGACGTCGACCACATCGTCCCGCAGAGCTTCCTGAAGGACGAC |
| | | AGCATCGACAACAAGGTCCTGACAAGAAGCGACAAGAACAGAGGAAAGAGCGACAACGTCCCGAGCGAAGAAGTCGTCAAGAAGA |
| | | TGAAGAACTACTGGAGACAGCTGCTGAACGCAAAGCTGATCACACAGAGAAAGTTCGACAACCTGACAAAGGCAGAGAGAGGAGG |
| | | ACTGAGCGAACTGGACAAGGCAGGATTCATCAAGAGACAGCTGGTCGAAACAAGACAGATCACAAAGCACGTCGCACAGATCCTG |
| | | GACAGCAGAATGAACACAAAGTACGACGAAAACGACAAGCTGATCAGAGAAGTCAAGGTCATCACACTGAAGAGCAAGCTGGTCA |
| | | GCGACTTCAGAAAGGACTTCCAGTTCTACAAGGTCAGAGAAATCAACAACTACCACCACGCACACGACGCATACCTGAACGCAGT |
| | | CGTCGGAACAGCACTGATCAAGAAGTACCCGAAGCTGGAAAGCGAATTCGTCTACGGAGACTACAAGGTCTACGACGTCAGAAAG |
| | | ATGATCGCAAAGAGCGAACAGGAAATCGGAAAGGCAACAGCAAAGTACTTCTTCTACAGCAACATCATGAACTTCTTCAAGACAG |
| | | AAATCACACTGGCAAACGGAGAAATCAGAAAGAGACCGCTGATCGAAACAAACGGAGAAACAGGAGAAATCGTCTGGGACAAGGG |
| | | AAGAGACTTCGCAACAGTCAGAAAGGTCCTGAGCATGCCGCAGGTCAACATCGTCAAGAAGACAGAAGTCCAGACAGGAGGATTC |
| | | AGCAAGGAAAGCATCCTGCCGAAGAGAAACAGCGACAAGCTGATCGCAAGAAAGAAGGACTGGGACCCGAAGAAGTACGGAGGAT |
| | | TCGACAGCCCGACAGTCGCATACAGCGTCCTGGTCGTCGCAAAGGTCGAAAAGGGAAAGAGCAAGAAGCTGAAGAGCGTCAAGGA |
| | | ACTGCTGGGAATCACAATCATGGAAAGAAGCAGCTTCGAAAAGAACCCGATCGACTTCCTGGAAGCAAAGGGATACAAGGAAGTC |
| | | AAGAAGGACCTGATCATCAAGCTGCCGAAGTACAGCCTGTTCGAACTGGAAAACGGAAGAAAGAGAATGCTGGCAAGCGCAGGAG |
| | | AACTGCAGAAGGGAAACGAACTGGCACTGCCGAGCAAGTACGTCAACTTCCTGTACCTGGCAAGCCACTACGAAAAGCTGAAGGG |
| | | AAGCCCGGAAGACAACGAACAGAAGCAGCTGTTCGTCGAACAGCACAAGCACTACCTGGACGAAATCATCGAACAGATCAGCGAA |
| | | TTCAGCAAGAGAGTCATCCTGGCAGACGCAAACCTGGACAAGGTCCTGAGCGCATACAACAAGCACAGAGACAAGCCGATCAGAG |
| | | AACAGGCAGAAAACATCATCCACCTGTTCACACTGACAAACCTGGGAGCACCGGCAGCATTCAAGTACTTCGACACAACAATCGA |
| | | CAGAAAGAGATACACAAGCACAAAGGAAGTCCTGGACGCAACACTGATCCACCAGAGCATCACAGGACTGTACGAAACAAGAATC |
| | | GACCTGAGCCAGCTGGGAGGAGACGGAGGAGGAAGCCCGAAGAAGAAGAGAAAGGTCTAG |
| ORF encoding Spy Cas9 | 802 | ATGGACAAGAAGTACTCCATCGGCCTGGACATCGGCACCAACTCCGTGGGCTGGGCCGTGATCACCGACGAGTACAAGGTGCCCT |
| | | CCAAGAAGTTCAAGGTGCTGGGCAACACCGACCGGCACTCCATCAAGAAGAACCTGATCGGCGCCCTGCTGTTCGACTCCGGCGA |
| | | GACCGCCGAGGCCACCCGGCTGAAGCGGACCGCCCGGCGGCGGTACACCCGGCGGAAGAACCGGATCTGCTACCTGCAGGAGATC |
| | | TTCTCCAACGAGATGGCCAAGGTGGACGACTCCTTCTTCCACCGGCTGGAGGAGTCCTTCCTGGTGGAGGAGGACAAGAAGCACG |
| | | AGCGGCACCCCATCTTCGGCAACATCGTGGACGAGGTGGCCTACCACGAGAAGTACCCCACCATCTACCACCTGCGGAAGAAGCT |
| | | GGTGGACTCCACCGACAAGGCCGACCTGCGGCTGATCTACCTGGCCCTGGCCCACATGATCAAGTTCCGGGGCCACTTCCTGATC |
| | | GAGGGCGACCTGAACCCCGACAACTCCGACGTGGACAAGCTGTTCATCCAGCTGGTGCAGACCTACAACCAGCTGTTCGAGGAGA |
| | | ACCCCATCAACGCCTCCGGCGTGGACGCCAAGGCCATCCTGTCCGCCCGGCTGTCCAAGTCCCGGCGGCTGGAGAACCTGATCGC |
| | | CCAGCTGCCCGGCGAGAAGAAGAACGGCCTGTTCGGCAACCTGATCGCCCTGTCCCTGGGCCTGACCCCCAACTTCAAGTCCAAC |
| | | TTCGACCTGGCCGAGGACGCCAAGCTGCAGCTGTCCAAGGACACCTACGACGACGACCTGGACAACCTGCTGGCCCAGATCGGCG |
| | | ACCAGTACGCCGACCTGTTCCTGGCCGCCAAGAACCTGTCCGACGCCATCCTGCTGTCCGACATCCTGCGGGTGAACACCGAGAT |
| | | CACCAAGGCCCCCCTGTCCGCCTCCATGATCAAGCGGTACGACGAGCACCACCAGGACCTGACCCTGCTGAAGGCCCTGGTGCGG |
| | | CAGCAGCTGCCCGAGAAGTACAAGGAGATCTTCTTCGACCAGTCCAAGAACGGCTACGCCGGCTACATCGACGGCGGCGCCTCCC |
| | | AGGAGGAGTTCTACAAGTTCATCAAGCCCATCCTGGAGAAGATGGACGGCACCGAGGAGCTGCTGGTGAAGCTGAACCGGGAGGA |
| | | CCTGCTGCGGAAGCAGCGGACCTTCGACAACGGCTCCATCCCCCACCAGATCCACCTGGGCGAGCTGCACGCCATCCTGCGGCGG |
| | | CAGGAGGACTTCTACCCCTTCCTGAAGGACAACCGGGAGAAGATCGAGAAGATCCTGACCTTCCGGATCCCCTACTACGTGGGCC |
| | | CCCTGGCCCGGGGCAACTCCCGGTTCGCCTGGATGACCCGGAAGTCCGAGGAGACCATCACCCCCTGGAACTTCGAGGAGGTGGT |
| | | GGACAAGGGCGCCTCCGCCCAGTCCTTCATCGAGCGGATGACCAACTTCGACAAGAACCTGCCCAACGAGAAGGTGCTGCCCAAG |
| | | CACTCCCTGCTGTACGAGTACTTCACCGTGTACAACGAGCTGACCAAGGTGAAGTACGTGACCGAGGGCATGCGGAAGCCCGCCT |
| | | TCCTGTCCGGCGAGCAGAAGAAGGCCATCGTGGACCTGCTGTTCAAGACCAACCGGAAGGTGACCGTGAAGCAGCTGAAGGAGGA |
| | | CTACTTCAAGAAGATCGAGTGCTTCGACTCCGTGGAGATCTCCGGCGTGGAGGACCGGTTCAACGCCTCCCTGGGCACCTACCAC |
| | | GACCTGCTGAAGATCATCAAGGACAAGGACTTCCTGGACAACGAGGAGAACGAGGACATCCTGGAGGACATCGTGCTGACCCTGA |
| | | CCCTGTTCGAGGACCGGGAGATGATCGAGGAGCGGCTGAAGACCTACGCCCACCTGTTCGACGACAAGGTGATGAAGCAGCTGAA |
| | | GCGGCGGCGGTACACCGGCTGGGGCCGGCTGTCCCGGAAGCTGATCAACGGCATCCGGGACAAGCAGTCCGGCAAGACCATCCTG |
| | | GACTTCCTGAAGTCCGACGGCTTCGCCAACCGGAACTTCATGCAGCTGATCCACGACGACTCCCTGACCTTCAAGGAGGACATCC |
| | | AGAAGGCCCAGGTGTCCGGCCAGGGCGACTCCCTGCACGAGCACATCGCCAACCTGGCCGGCTCCCCCGCCATCAAGAAGGGCAT |
| | | CCTGCAGACCGTGAAGGTGGTGGACGAGCTGGTGAAGGTGATGGGCCGGCACAAGCCCGAGAACATCGTGATCGAGATGGCCCGG |
| | | GAGAACCAGACCACCCAGAAGGGCCAGAAGAACTCCCGGGAGCGGATGAAGCGGATCGAGGAGGGCATCAAGGAGCTGGGCTCCC |
| | | AGATCCTGAAGGAGCACCCCGTGGAGAACACCCAGCTGCAGAACGAGAAGCTGTACCTGTACTACCTGCAGAACGGCCGGGACAT |
| | | GTACGTGGACCAGGAGCTGGACATCAACCGGCTGTCCGACTACGACGTGGACCACATCGTGCCCCAGTCCTTCCTGAAGGACGAC |
| | | TCCATCGACAACAAGGTGCTGACCCGGTCCGACAAGAACCGGGGCAAGTCCGACAACGTGCCCTCCGAGGAGGTGGTGAAGAAGA |
| | | TGAAGAACTACTGGCGGCAGCTGCTGAACGCCAAGCTGATCACCCAGCGGAAGTTCGACAACCTGACCAAGGCCGAGCGGGGCGG |
| | | CCTGTCCGAGCTGGACAAGGCCGGCTTCATCAAGCGGCAGCTGGTGGAGACCCGGCAGATCACCAAGCACGTGGCCCAGATCCTG |
| | | GACTCCCGGATGAACACCAAGTACGACGAGAACGACAAGCTGATCCGGGAGGTGAAGGTGATCACCCTGAAGTCCAAGCTGGTGT |
| | | CCGACTTCCGGAAGGACTTCCAGTTCTACAAGGTGCGGGAGATCAACAACTACCACCACGCCCACGACGCCTACCTGAACGCCGT |
| | | GGTGGGCACCGCCCTGATCAAGAAGTACCCCAAGCTGGAGTCCGAGTTCGTGTACGGCGACTACAAGGTGTACGACGTGCGGAAG |
| | | ATGATCGCCAAGTCCGAGCAGGAGATCGGCAAGGCCACCGCCAAGTACTTCTTCTACTCCAACATCATGAACTTCTTCAAGACCG |
| | | AGATCACCCTGGCCAACGGCGAGATCCGGAAGCGGCCCCTGATCGAGACCAACGGCGAGACCGGCGAGATCGTGTGGGACAAGGG |
| | | CCGGGACTTCGCCACCGTGCGGAAGGTGCTGTCCATGCCCCAGGTGAACATCGTGAAGAAGACCGAGGTGCAGACCGGCGGCTTC |
| | | TCCAAGGAGTCCATCCTGCCCAAGCGGAACTCCGACAAGCTGATCGCCCGGAAGAAGGACTGGGACCCCAAGAAGTACGGCGGCT |
| | | TCGACTCCCCCACCGTGGCCTACTCCGTGCTGGTGGTGGCCAAGGTGGAGAAGGGCAAGTCCAAGAAGCTGAAGTCCGTGAAGGA |
| | | GCTGCTGGGCATCACCATCATGGAGCGGTCCTCCTTCGAGAAGAACCCCATCGACTTCCTGGAGGCCAAGGGCTACAAGGAGGTG |
| | | AAGAAGGACCTGATCATCAAGCTGCCCAAGTACTCCCTGTTCGAGCTGGAGAACGGCCGGAAGCGGATGCTGGCCTCCGCCGGCG |
| | | AGCTGCAGAAGGGCAACGAGCTGGCCCTGCCCTCCAAGTACGTGAACTTCCTGTACCTGGCCTCCCACTACGAGAAGCTGAAGGG |
| | | CTCCCCCGAGGACAACGAGCAGAAGCAGCTGTTCGTGGAGCAGCACAAGCACTACCTGGACGAGATCATCGAGCAGATCTCCGAG |
| | | TTCTCCAAGCGGGTGATCCTGGCCGACGCCAACCTGGACAAGGTGCTGTCCGCCTACAACAAGCACCGGGACAAGCCCATCCGGG |
| | | AGCAGGCCGAGAACATCATCCACCTGTTCACCCTGACCAACCTGGGCGCCCCCGCCGCCTTCAAGTACTTCGACACCACCATCGA |
| | | CCGGAAGCGGTACACCTCCACCAAGGAGGTGCTGGACGCCACCCTGATCCACCAGTCCATCACCGGCCTGTACGAGACCCGGATC |
| | | GACCTGTCCCAGCTGGGCGGCGACGGCGGCGGCTCCCCCAAGAAGAAGCGGAAGGTGTGA |
| Open reading frame | 803 | AUGGACAAGAAGUACUCCAUCGGCCUGGACAUCGGCACCAACUCCGUGGGCUGGGCCGUGAUCACCGACGAGUACAAGGUGCCCU |
| for Spy Cas9 with | | CCAAGAAGUUCAAGGUGCUGGGCAACACCGACCGGCACUCCAUCAAGAAGAACCUGAUCGGCGCCCUGCUGUUCGACUCCGGCGA |
| Hibit tag | | GACCGCCGAGGCCACCCGGCUGAAGCGGACCGCCCGGCGGCGGUACACCCGGCGGAAGAACCGGAUCUGCUACCUGCAGGAGAUC |
| | | UUCUCCAACGAGAUGGCCAAGGUGGACGACUCCUUCUUCCACCGGCUGGAGGAGUCCUUCCUGGUGGAGGAGGACAAGAAGCACG |
| | | AGCGGCACCCCAUCUUCGGCAACAUCGUGGACGAGGUGGCCUACCACGAGAAGUACCCCACCAUCUACCACCUGCGGAAGAAGCU |
| | | GGUGGACUCCACCGACAAGGCCGACCUGCGGCUGAUCUACCUGGCCCUGGCCCACAUGAUCAAGUUCCGGGGCCACUUCCUGAUC |
| | | GAGGGCGACCUGAACCCCGACAACUCCGACGUGGACAAGCUGUUCAUCCAGCUGGUGCAGACCUACAACCAGCUGUUCGAGGAGA |
| | | ACCCCAUCAACGCCUCCGGCGUGGACGCCAAGGCCAUCCUGUCCGCCCGGCUGUCCAAGUCCCGGCGGCUGGAGAACCUGAUCGC |
| | | CCAGCUGCCCGGCGAGAAGAAGAACGGCCUGUUCGGCAACCUGAUCGCCCUGUCCCUGGGCCUGACCCCCAACUUCAAGUCCAAC |
| | | UUCGACCUGGCCGAGGACGCCAAGCUGCAGCUGUCCAAGGACACCUACGACGACGACCUGGACAACCUGCUGGCCCAGAUCGGCG |
| | | ACCAGUACGCCGACCUGUUCCUGGCCGCCAAGAACCUGUCCGACGCCAUCCUGCUGUCCGACAUCCUGCGGGUGAACACCGAGAU |
| | | CACCAAGGCCCCCCUGUCCGCCUCCAUGAUCAAGCGGUACGACGAGCACCACCAGGACCUGACCCUGCUGAAGGCCCUGGUGCGG |
| | | CAGCAGCUGCCCGAGAAGUACAAGGAGAUCUUCUUCGACCAGUCCAAGAACGGCUACGCCGGCUACAUCGACGGCGGCGCCUCCC |
| | | AGGAGGAGUUCUACAAGUUCAUCAAGCCCAUCCUGGAGAAGAUGGACGGCACCGAGGAGCUGCUGGUGAAGCUGAACCGGGAGGA |
| | | CCUGCUGCGGAAGCAGCGGACCUUCGACAACGGCUCCAUCCCCCACCAGAUCCACCUGGGCGAGCUGCACGCCAUCCUGCGGCGG |
| | | CAGGAGGACUUCUACCCCUUCCUGAAGGACAACCGGGAGAAGAUCGAGAAGAUCCUGACCUUCCGGAUCCCCUACUACGUGGGCC |
| | | CCCUGGCCCGGGGCAACUCCCGGUUCGCCUGGAUGACCCGGAAGUCCGAGGAGACCAUCACCCCCUGGAACUUCGAGGAGGUGGU |
| | | GGACAAGGGCGCCUCCGCCCAGUCCUUCAUCGAGCGGAUGACCAACUUCGACAAGAACCUGCCCAACGAGAAGGUGCUGCCCAAG |
| | | CACUCCCUGCUGUACGAGUACUUCACCGUGUACAACGAGCUGACCAAGGUGAAGUACGUGACCGAGGGCAUGCGGAAGCCCGCCU |
| | | UCCUGUCCGGCGAGCAGAAGAAGGCCAUCGUGGACCUGCUGUUCAAGACCAACCGGAAGGUGACCGUGAAGCAGCUGAAGGAGGA |
| | | CUACUUCAAGAAGAUCGAGUGCUUCGACUCCGUGGAGAUCUCCGGCGUGGAGGACCGGUUCAACGCCUCCCUGGGCACCUACCAC |
| | | GACCUGCUGAAGAUCAUCAAGGACAAGGACUUCCUGGACAACGAGGAGAACGAGGACAUCCUGGAGGACAUCGUGCUGACCCUGA |
| | | CCCUGUUCGAGGACCGGGAGAUGAUCGAGGAGCGGCUGAAGACCUACGCCCACCUGUUCGACGACAAGGUGAUGAAGCAGCUGAA |
| | | GCGGCGGCGGUACACCGGCUGGGGCCGGCUGUCCCGGAAGCUGAUCAACGGCAUCCGGGACAAGCAGUCCGGCAAGACCAUCCUG |
| | | GACUUCCUGAAGUCCGACGGCUUCGCCAACCGGAACUUCAUGCAGCUGAUCCACGACGACUCCCUGACCUUCAAGGAGGACAUCC |
| | | AGAAGGCCCAGGUGUCCGGCCAGGGCGACUCCCUGCACGAGCACAUCGCCAACCUGGCCGGCUCCCCCGCCAUCAAGAAGGGCAU |
| | | CCUGCAGACCGUGAAGGUGGUGGACGAGCUGGUGAAGGUGAUGGGCCGGCACAAGCCCGAGAACAUCGUGAUCGAGAUGGCCCGG |
| | | GAGAACCAGACCACCCAGAAGGGCCAGAAGAACUCCCGGGAGCGGAUGAAGCGGAUCGAGGAGGGCAUCAAGGAGCUGGGCUCCC |
| | | AGAUCCUGAAGGAGCACCCCGUGGAGAACACCCAGCUGCAGAACGAGAAGCUGUACCUGUACUACCUGCAGAACGGCCGGGACAU |
| | | GUACGUGGACCAGGAGCUGGACAUCAACCGGCUGUCCGACUACGACGUGGACCACAUCGUGCCCCAGUCCUUCCUGAAGGACGAC |
| | | UCCAUCGACAACAAGGUGCUGACCCGGUCCGACAAGAACCGGGGCAAGUCCGACAACGUGCCCUCCGAGGAGGUGGUGAAGAAGA |
| | | UGAAGAACUACUGGCGGCAGCUGCUGAACGCCAAGCUGAUCACCCAGCGGAAGUUCGACAACCUGACCAAGGCCGAGCGGGGCGG |
| | | CCUGUCCGAGCUGGACAAGGCCGGCUUCAUCAAGCGGCAGCUGGUGGAGACCCGGCAGAUCACCAAGCACGUGGCCCAGAUCCUG |
| | | GACUCCCGGAUGAACACCAAGUACGACGAGAACGACAAGCUGAUCCGGGAGGUGAAGGUGAUCACCCUGAAGUCCAAGCUGGUGU |
| | | CCGACUUCCGGAAGGACUUCCAGUUCUACAAGGUGCGGGAGAUCAACAACUACCACCACGCCCACGACGCCUACCUGAACGCCGU |
| | | GGUGGGCACCGCCCUGAUCAAGAAGUACCCCAAGCUGGAGUCCGAGUUCGUGUACGGCGACUACAAGGUGUACGACGUGCGGAAG |
| | | AUGAUCGCCAAGUCCGAGCAGGAGAUCGGCAAGGCCACCGCCAAGUACUUCUUCUACUCCAACAUCAUGAACUUCUUCAAGACCG |
| | | AGAUCACCCUGGCCAACGGCGAGAUCCGGAAGCGGCCCCUGAUCGAGACCAACGGCGAGACCGGCGAGAUCGUGUGGGACAAGGG |
| | | CCGGGACUUCGCCACCGUGCGGAAGGUGCUGUCCAUGCCCCAGGUGAACAUCGUGAAGAAGACCGAGGUGCAGACCGGCGGCUUC |
| | | UCCAAGGAGUCCAUCCUGCCCAAGCGGAACUCCGACAAGCUGAUCGCCCGGAAGAAGGACUGGGACCCCAAGAAGUACGGCGGCU |
| | | UCGACUCCCCCACCGUGGCCUACUCCGUGCUGGUGGUGGCCAAGGUGGAGAAGGGCAAGUCCAAGAAGCUGAAGUCCGUGAAGGA |
| | | GCUGCUGGGCAUCACCAUCAUGGAGCGGUCCUCCUUCGAGAAGAACCCCAUCGACUUCCUGGAGGCCAAGGGCUACAAGGAGGUG |
| | | AAGAAGGACCUGAUCAUCAAGCUGCCCAAGUACUCCCUGUUCGAGCUGGAGAACGGCCGGAAGCGGAUGCUGGCCUCCGCCGGCG |
| | | AGCUGCAGAAGGGCAACGAGCUGGCCCUGCCCUCCAAGUACGUGAACUUCCUGUACCUGGCCUCCCACUACGAGAAGCUGAAGGG |
| | | CUCCCCCGAGGACAACGAGCAGAAGCAGCUGUUCGUGGAGCAGCACAAGCACUACCUGGACGAGAUCAUCGAGCAGAUCUCCGAG |
| | | UUCUCCAAGCGGGUGAUCCUGGCCGACGCCAACCUGGACAAGGUGCUGUCCGCCUACAACAAGCACCGGGACAAGCCCAUCCGGG |
| | | AGCAGGCCGAGAACAUCAUCCACCUGUUCACCCUGACCAACCUGGGCGCCCCCGCCGCCUUCAAGUACUUCGACACCACCAUCGA |
| | | CCGGAAGCGGUACACCUCCACCAAGGAGGUGCUGGACGCCACCCUGAUCCACCAGUCCAUCACCGGCCUGUACGAGACCCGGAUC |
| | | GACCUGUCCCAGCUGGGCGGCGACGGCGGCGGCUCCCCCAAGAAGAAGCGGAAGGUGUCCGAGUCCGCCACCCCCGAGUCCGUGU |
| | | CCGGCUGGCGGCUGUUCAAGAAGAUCUCCUGA |
| Open Reading frame | 804 | AUGgaggccucccccgccuccggcccccggcaccugauggacccccacaucuucaccuccAACUUCAACAACggcAUCggccggC |
| for BC22n | | ACAAGaccUACCUGUGCUACgagguggagcggCUGGACAACggcaccuccgugAAGAUGGACCAGCACcggggcUUCCUGCACAA |
| | | CCAGgccAAGAACCUGCUGUGCggcUUCUACggccggCACgccgagCUGcggUUCCUGGACCUGgugcccuccCUGCAGCUGGAC |
| | | cccgccCAGAUCUACcgggugaccUGGUUCAUCuccUGGucccccUGCUUCuccUGGggcUGCgccggcgaggugcgggccUUCC |
| | | UGCAGgagAACaccCACgugcggCUGcggAUCUUCgccgcccggAUCUACGACUACGACcccCUGUACAAGgaggccCUGCAGAU |
| | | GCUGcggGACgccggcgccCAGguguccAUCAUGaccUACGACgagUUCAAGCACUGCUGGGACaccUUCgugGACCACCAGggc |
| | | UGCcccUUCCAGcccUGGGACggcCUGGACgagCACuccCAGgccCUGuccggccggCUGcgggccAUCCUGCAGAACCAGggcA |
| | | ACuccggcuccgagacccccggcaccuccgaguccgccacccccgaguccgacaagaaguacuccaucggccuggCcaucggcac |
| | | caacuccgugggcugggccgugaucaccgacgaguacaaggugcccuccaagaaguucaaggugcugggcaacaccgaccggcac |
| | | uccaucaagaagaaccugaucggcgcccugcuguucgacuccggcgagaccgccgaggccacccggcugaagcggaccgcccggc |
| | | ggcgguacacccggcggaagaaccggaucugcuaccugcaggagaucuucuccaacgagauggccaagguggacgacuccuucuu |
| | | ccaccggcuggaggaguccuuccugguggaggaggacaagaagcacgagcggcaccccaucuucggcaacaucguggacgaggug |
| | | gccuaccacgagaaguaccccaccaucuaccaccugcggaagaagcugguggacuccaccgacaaggccgaccugcggcugaucu |
| | | accuggcccuggcccacaugaucaaguuccggggccacuuccugaucgagggcgaccugaaccccgacaacuccgacguggacaa |
| | | gcuguucauccagcuggugcagaccuacaaccagcuguucgaggagaaccccaucaacgccuccggcguggacgccaaggccauc |
| | | cuguccgcccggcuguccaagucccggcggcuggagaaccugaucgcccagcugcccggcgagaagaagaacggccuguucggca |
| | | accugaucgcccugucccugggccugacccccaacuucaaguccaacuucgaccuggccgaggacgccaagcugcagcuguccaa |
| | | ggacaccuacgacgacgaccuggacaaccugcuggcccagaucggcgaccaguacgccgaccuguuccuggccgccaagaaccug |
| | | uccgacgccauccugcuguccgacauccugcgggugaacaccgagaucaccaaggccccccuguccgccuccaugaucaagcggu |
| | | acgacgagcaccaccaggaccugacccugcugaaggcccuggugcggcagcagcugcccgagaaguacaaggagaucuucuucga |
| | | ccaguccaagaacggcuacgccggcuacaucgacggcggcgccucccaggaggaguucuacaaguucaucaagcccauccuggag |
| | | aagauggacggcaccgaggagcugcuggugaagcugaaccgggaggaccugcugcggaagcagcggaccuucgacaacggcucca |
| | | ucccccaccagauccaccugggcgagcugcacgccauccugcggcggcaggaggacuucuaccccuuccugaaggacaaccggga |
| | | gaagaucgagaagauccugaccuuccggauccccuacuacgugggcccccuggcccggggcaacucccgguucgccuggaugacc |
| | | cggaaguccgaggagaccaucacccccuggaacuucgaggaggugguggacaagggcgccuccgcccaguccuucaucgagcgga |
| | | ugaccaacuucgacaagaaccugcccaacgagaaggugcugcccaagcacucccugcuguacgaguacuucaccguguacaacga |
| | | gcugaccaaggugaaguacgugaccgagggcaugcggaagcccgccuuccuguccggcgagcagaagaaggccaucguggaccug |
| | | cuguucaagaccaaccggaaggugaccgugaagcagcugaaggaggacuacuucaagaagaucgagugcuucgacuccguggaga |
| | | ucuccggcguggaggaccgguucaacgccucccugggcaccuaccacgaccugcugaagaucaucaaggacaaggacuuccugga |
| | | caacgaggagaacgaggacauccuggaggacaucgugcugacccugacccuguucgaggaccgggagaugaucgaggagcggcug |
| | | aagaccuacgcccaccuguucgacgacaaggugaugaagcagcugaagcggcggcgguacaccggcuggggccggcugucccgga |
| | | agcugaucaacggcauccgggacaagcaguccggcaagaccauccuggacuuccugaaguccgacggcuucgccaaccggaacuu |
| | | caugcagcugauccacgacgacucccugaccuucaaggaggacauccagaaggcccagguguccggccagggcgacucccugcac |
| | | gagcacaucgccaaccuggccggcucccccgccaucaagaagggcauccugcagaccgugaaggugguggacgagcuggugaagg |
| | | ugaugggccggcacaagcccgagaacaucgugaucgagauggcccgggagaaccagaccacccagaagggccagaagaacucccg |
| | | ggagcggaugaagcggaucgaggagggcaucaaggagcugggcucccagauccugaaggagcaccccguggagaacacccagcug |
| | | cagaacgagaagcuguaccuguacuaccugcagaacggccgggacauguacguggaccaggagcuggacaucaaccggcuguccg |
| | | acuacgacguggaccacaucgugccccaguccuuccugaaggacgacuccaucgacaacaaggugcugacccgguccgacaagaa |
| | | ccggggcaaguccgacaacgugcccuccgaggagguggugaagaagaugaagaacuacuggcggcagcugcugaacgccaagcug |
| | | aucacccagcggaaguucgacaaccugaccaaggccgagcggggCggccuguccgagcuggacaaggccggcuucaucaagcggc |
| | | agcugguggagacccggcagaucaccaagcacguggcccagauccuggacucccggaugaacaccaaguacgacgagaacgacaa |
| | | gcugauccgggaggugaaggugaucacccugaaguccaagcugguguccgacuuccggaaggacuuccaguucuacaaggugcgg |
| | | gagaucaacaacuaccaccacgcccacgacgccuaccugaacgccguggugggcaccgcccugaucaagaaguaccccaagcugg |
| | | aguccgaguucguguacggcgacuacaagguguacgacgugcggaagaugaucgccaaguccgagcaggagaucggcaaggccac |
| | | cgccaaguacuucuucuacuccaacaucaugaacuucuucaagaccgagaucacccuggccaacggcgagauccggaagcggccc |
| | | cugaucgagaccaacggcgagaccggcgagaucgugugggacaagggccgggacuucgccaccgugcggaaggugcuguccaugc |
| | | cccaggugaacaucgugaagaagaccgaggugcagaccggcggcuucuccaaggaguccauccugcccaagcggaacuccgacaa |
| | | gcugaucgcccggaagaaggacugggaccccaagaaguacggcggcuucgacucccccaccguggccuacuccgugcugguggug |
| | | gccaagguggagaagggcaaguccaagaagcugaaguccgugaaggagcugcugggcaucaccaucauggagcgguccuccuucg |
| | | agaagaaccccaucgacuuccuggaggccaagggcuacaaggaggugaagaaggaccugaucaucaagcugcccaaguacucccu |
| | | guucgagcuggagaacggccggaagcggaugcuggccuccgccggcgagcugcagaagggcaacgagcuggcccugcccuccaag |
| | | uacgugaacuuccuguaccuggccucccacuacgagaagcugaagggcucccccgaggacaacgagcagaagcagcuguucgugg |
| | | agcagcacaagcacuaccuggacgagaucaucgagcagaucuccgaguucuccaagcgggugauccuggccgacgccaaccugga |
| | | caaggugcuguccgccuacaacaagcaccgggacaagcccauccgggagcaggccgagaacaucauccaccuguucacccugacc |
| | | aaccugggcgcccccgccgccuucaaguacuucgacaccaccaucgaccggaagcgguacaccuccaccaaggaggugcuggacg |
| | | ccacccugauccaccaguccaucaccggccuguacgagacccggaucgaccugucccagcugggcggcgacggcggcggcucccc |
| | | caagaagaagcggaaggugUgA |
| Open reading frame | 805 | AUGgaggccucccccgccuccggcccccggcaccugauggacccccacaucuucaccuccAACUUCAACAACggcAUCggccggC |
| for BC22n with Hibit | | ACAAGaccUACCUGUGCUACgagguggagcggCUGGACAACggcaccuccgugAAGAUGGACCAGCACcggggcUUCCUGCACAA |
| tag | | CCAGgccAAGAACCUGCUGUGCggcUUCUACggccggCACgccgagCUGcggUUCCUGGACCUGgugcccuccCUGCAGCUGGAC |
| | | cccgccCAGAUCUACcgggugaccUGGUUCAUCuccUGGucccccUGCUUCuccUGGggcUGCgccggcgaggugcgggccUUCC |
| | | UGCAGgagAACaccCACgugcggCUGcggAUCUUCgccgcccggAUCUACGACUACGACcccCUGUACAAGgaggccCUGCAGAU |
| | | GCUGcggGACgccggcgccCAGguguccAUCAUGaccUACGACgagUUCAAGCACUGCUGGGACaccUUCgugGACCACCAGggc |
| | | UGCcccUUCCAGcccUGGGACggcCUGGACgagCACuccCAGgccCUGuccggccggCUGcgggccAUCCUGCAGAACCAGggcA |
| | | ACuccggcuccgagacccccggcaccuccgaguccgccacccccgaguccgacaagaaguacuccaucggccuggCcaucggcac |
| | | caacuccgugggcugggccgugaucaccgacgaguacaaggugcccuccaagaaguucaaggugcugggcaacaccgaccggcac |
| | | uccaucaagaagaaccugaucggcgcccugcuguucgacuccggcgagaccgccgaggccacccggcugaagcggaccgcccggc |
| | | ggcgguacacccggcggaagaaccggaucugcuaccugcaggagaucuucuccaacgagauggccaagguggacgacuccuucuu |
| | | ccaccggcuggaggaguccuuccugguggaggaggacaagaagcacgagcggcaccccaucuucggcaacaucguggacgaggug |
| | | gccuaccacgagaaguaccccaccaucuaccaccugcggaagaagcugguggacuccaccgacaaggccgaccugcggcugaucu |
| | | accuggcccuggcccacaugaucaaguuccggggccacuuccugaucgagggcgaccugaaccccgacaacuccgacguggacaa |
| | | gcuguucauccagcuggugcagaccuacaaccagcuguucgaggagaaccccaucaacgccuccggcguggacgccaaggccauc |
| | | cuguccgcccggcuguccaagucccggcggcuggagaaccugaucgcccagcugcccggcgagaagaagaacggccuguucggca |
| | | accugaucgcccugucccugggccugacccccaacuucaaguccaacuucgaccuggccgaggacgccaagcugcagcuguccaa |
| | | ggacaccuacgacgacgaccuggacaaccugcuggcccagaucggcgaccaguacgccgaccuguuccuggccgccaagaaccug |
| | | uccgacgccauccugcuguccgacauccugcgggugaacaccgagaucaccaaggccccccuguccgccuccaugaucaagcggu |
| | | acgacgagcaccaccaggaccugacccugcugaaggcccuggugcggcagcagcugcccgagaaguacaaggagaucuucuucga |
| | | ccaguccaagaacggcuacgccggcuacaucgacggcggcgccucccaggaggaguucuacaaguucaucaagcccauccuggag |
| | | aagauggacggcaccgaggagcugcuggugaagcugaaccgggaggaccugcugcggaagcagcggaccuucgacaacggcucca |
| | | ucccccaccagauccaccugggcgagcugcacgccauccugcggcggcaggaggacuucuaccccuuccugaaggacaaccggga |
| | | gaagaucgagaagauccugaccuuccggauccccuacuacgugggcccccuggcccggggcaacucccgguucgccuggaugacc |
| | | cggaaguccgaggagaccaucacccccuggaacuucgaggaggugguggacaagggcgccuccgcccaguccuucaucgagcgga |
| | | ugaccaacuucgacaagaaccugcccaacgagaaggugcugcccaagcacucccugcuguacgaguacuucaccguguacaacga |
| | | gcugaccaaggugaaguacgugaccgagggcaugcggaagcccgccuuccuguccggcgagcagaagaaggccaucguggaccug |
| | | cuguucaagaccaaccggaaggugaccgugaagcagcugaaggaggacuacuucaagaagaucgagugcuucgacuccguggaga |
| | | ucuccggcguggaggaccgguucaacgccucccugggcaccuaccacgaccugcugaagaucaucaaggacaaggacuuccugga |
| | | caacgaggagaacgaggacauccuggaggacaucgugcugacccugacccuguucgaggaccgggagaugaucgaggagcggcug |
| | | aagaccuacgcccaccuguucgacgacaaggugaugaagcagcugaagcggcggcgguacaccggcuggggccggcugucccgga |
| | | agcugaucaacggcauccgggacaagcaguccggcaagaccauccuggacuuccugaaguccgacggcuucgccaaccggaacuu |
| | | caugcagcugauccacgacgacucccugaccuucaaggaggacauccagaaggcccagguguccggccagggcgacucccugcac |
| | | gagcacaucgccaaccuggccggcucccccgccaucaagaagggcauccugcagaccgugaaggugguggacgagcuggugaagg |
| | | ugaugggccggcacaagcccgagaacaucgugaucgagauggcccgggagaaccagaccacccagaagggccagaagaacucccg |
| | | ggagcggaugaagcggaucgaggagggcaucaaggagcugggcucccagauccugaaggagcaccccguggagaacacccagcug |
| | | cagaacgagaagcuguaccuguacuaccugcagaacggccgggacauguacguggaccaggagcuggacaucaaccggcuguccg |
| | | acuacgacguggaccacaucgugccccaguccuuccugaaggacgacuccaucgacaacaaggugcugacccgguccgacaagaa |
| | | ccggggcaaguccgacaacgugcccuccgaggagguggugaagaagaugaagaacuacuggcggcagcugcugaacgccaagcug |
| | | aucacccagcggaaguucgacaaccugaccaaggccgagcggggcggccuguccgagcuggacaaggccggcuucaucaagcggc |
| | | agcugguggagacccggcagaucaccaagcacguggcccagauccuggacucccggaugaacaccaaguacgacgagaacgacaa |
| | | gcugauccgggaggugaaggugaucacccugaaguccaagcugguguccgacuuccggaaggacuuccaguucuacaaggugcgg |
| | | gagaucaacaacuaccaccacgcccacgacgccuaccugaacgccguggugggcaccgcccugaucaagaaguaccccaagcugg |
| | | aguccgaguucguguacggcgacuacaagguguacgacgugcggaagaugaucgccaaguccgagcaggagaucggcaaggccac |
| | | cgccaaguacuucuucuacuccaacaucaugaacuucuucaagaccgagaucacccuggccaacggcgagauccggaagcggccc |
| | | cugaucgagaccaacggcgagaccggcgagaucgugugggacaagggccgggacuucgccaccgugcggaaggugcuguccaugc |
| | | cccaggugaacaucgugaagaagaccgaggugcagaccggcggcuucuccaaggaguccauccugcccaagcggaacuccgacaa |
| | | gcugaucgcccggaagaaggacugggaccccaagaaguacggcggcuucgacucccccaccguggccuacuccgugcugguggug |
| | | gccaagguggagaagggcaaguccaagaagcugaaguccgugaaggagcugcugggcaucaccaucauggagcgguccuccuucg |
| | | agaagaaccccaucgacuuccuggaggccaagggcuacaaggaggugaagaaggaccugaucaucaagcugcccaaguacucccu |
| | | guucgagcuggagaacggccggaagcggaugcuggccuccgccggcgagcugcagaagggcaacgagcuggcccugcccuccaag |
| | | uacgugaacuuccuguaccuggccucccacuacgagaagcugaagggcucccccgaggacaacgagcagaagcagcuguucgugg |
| | | agcagcacaagcacuaccuggacgagaucaucgagcagaucuccgaguucuccaagcgggugauccuggccgacgccaaccugga |
| | | caaggugcuguccgccuacaacaagcaccgggacaagcccauccgggagcaggccgagaacaucauccaccuguucacccugacc |
| | | aaccugggcgcccccgccgccuucaaguacuucgacaccaccaucgaccggaagcgguacaccuccaccaaggaggugcuggacg |
| | | ccacccugauccaccaguccaucaccggccuguacgagacccggaucgaccugucccagcugggcggcgacggcggcggcucccc |
| | | caagaagaagcggaagguguccgaguccgccacccccgaguccguguccggcuggcggcuguucaagaagaucuccUgA |
| ORF encoding Spy Cas9 | 806 | ATGgaggccTcccccgccTccggcccccggcaccTgaTggacccccacaTcTTcaccTccAACTTCAACAACggcATCggccggC |
| | | ACAAGaccTACCTGTGCTACgaggTggagcggCTGGACAACggcaccTccgTgAAGATGGACCAGCACcggggcTTCCTGCACAA |
| | | CCAGgccAAGAACCTGCTGTGCggcTTCTACggccggCACgccgagCTGcggTTCCTGGACCTGgTgcccTccCTGCAGCTGGAC |
| | | cccgccCAGATCTACcgggTgaccTGGTTCATCTccTGGTCCCCCTGCTTCTccTGGggcTGCgccggcgaggTgcgggccTTCC |
| | | TGCAGgagAACaccCACgTgcggCTGcggATCTTCgccgcccggATCTACGACTACGACcccCTGTACAAGgaggccCTGCAGAT |
| | | GCTGcggGACgccggcgccCAGgTgTccATCATGaccTACGACgagTTCAAGCACTGCTGGGACaccTTCgTgGACCACCAGggc |
| | | TGCcccTTCCAGcccTGGGACggcCTGGACgagCACTccCAGgccCTGTccggccggCTGcgggccATCCTGCAGAACCAGggcA |
| | | ACTccggcTccgagacccccggcaccTccgagTccgccacccccgagTccgacaagaagTacTccaTcggccTggCcaTcggcac |
| | | caacTccgTgggcTgggccgTgaTcaccgacgagTacaaggTgcccTccaagaagTTcaaggTgcTgggcaacaccgaccggcac |
| | | TccaTcaagaagaaccTgaTcggcgcccTgcTgTTcgacTccggcgagaccgccgaggccacccggcTgaagcggaccgcccggc |
| | | ggcggTacacccggcggaagaaccggaTcTgcTaccTgcaggagaTcTTcTccaacgagaTggccaaggTggacgacTccTTcTT |
| | | ccaccggcTggaggagTccTTccTggTggaggaggacaagaagcacgagcggcaccccaTcTTcggcaacaTcgTggacgaggTg |
| | | gccTaccacgagaagTaccccaccaTcTaccaccTgcggaagaagcTggTggacTccaccgacaaggccgaccTgcggcTgaTcT |
| | | accTggcccTggcccacaTgaTcaagTTccggggccacTTccTgaTcgagggcgaccTgaaccccgacaacTccgacgTggacaa |
| | | gcTgTTcaTccagcTggTgcagaccTacaaccagcTgTTcgaggagaaccccaTcaacgccTccggcgTggacgccaaggccaTc |
| | | cTgTccgcccggcTgTccaagTcccggcggcTggagaaccTgaTcgcccagcTgcccggcgagaagaagaacggccTgTTcggca |
| | | accTgaTcgcccTgTcccTgggccTgacccccaacTTcaagTccaacTTcgaccTggccgaggacgccaagcTgcagcTgTccaa |
| | | ggacaccTacgacgacgaccTggacaaccTgcTggcccagaTcggcgaccagTacgccgaccTgTTccTggccgccaagaaccTg |
| | | TccgacgccaTccTgcTgTccgacaTccTgcgggTgaacaccgagaTcaccaaggccccccTgTccgccTccaTgaTcaagcggT |
| | | acgacgagcaccaccaggaccTgacccTgcTgaaggcccTggTgcggcagcagcTgcccgagaagTacaaggagaTcTTcTTcga |
| | | ccagTccaagaacggcTacgccggcTacaTcgacggcggcgccTcccaggaggagTTcTacaagTTcaTcaagcccaTccTggag |
| | | aagaTggacggcaccgaggagcTgcTggTgaagcTgaaccgggaggaccTgcTgcggaagcagcggaccTTcgacaacggcTcca |
| | | TcccccaccagaTccaccTgggcgagcTgcacgccaTccTgcggcggcaggaggacTTcTaccccTTccTgaaggacaaccggga |
| | | gaagaTcgagaagaTccTgaccTTccggaTccccTacTacgTgggcccccTggcccggggcaacTcccggITcgccTggaTgacc |
| | | cggaagTccgaggagaccaTcacccccTggaacTTcgaggaggTggTggacaagggcgccTccgcccagTccTTcaTcgagcgga |
| | | TgaccaacTTcgacaagaaccTgcccaacgagaaggTgcTgcccaagcacTcccTgcTgTacgagTacTTcaccgTgTacaacga |
| | | gcTgaccaaggTgaagTacgTgaccgagggcaTgcggaagcccgccTTccTgTccggcgagcagaagaaggccaTcgTggaccTg |
| | | cTgTTcaagaccaaccggaaggTgaccgTgaagcagcTgaaggaggacTacTTcaagaagaTcgagTgcTTcgacTccgTggaga |
| | | TcTccggcgTggaggaccggTTcaacgccTcccTgggcaccTaccacgaccTgcTgaagaTcaTcaaggacaaggacTTccTgga |
| | | caacgaggagaacgaggacaTccTggaggacaTcgTgcTgacccTgacccTgTTcgaggaccgggagaTgaTcgaggagcggcTg |
| | | aagaccTacgcccaccTgTTcgacgacaaggTgaTgaagcagcTgaagcggcggcggTacaccggcTggggccggcTgTcccgga |
| | | agcTgaTcaacggcaTccgggacaagcagTccggcaagaccaTccTggacTTccTgaagTccgacggcTTcgccaaccggaacTT |
| | | caTgcagcTgaTccacgacgacTcccTgaccTTcaaggaggacaTccagaaggcccaggTgTccggccagggcgacTcccTgcac |
| | | gagcacaTcgccaaccTggccggcTcccccgccaTcaagaagggcaTccTgcagaccgTgaaggTggTggacgagcTggTgaagg |
| | | TgaTgggccggcacaagcccgagaacaTcgTgaTcgagaTggcccgggagaaccagaccacccagaagggccagaagaacTcccg |
| | | ggagcggaTgaagcggaTcgaggagggcaTcaaggagcTgggcTcccagaTccTgaaggagcaccccgTggagaacacccagcTg |
| | | cagaacgagaagcTgTaccTgTacTaccTgcagaacggccgggacaTgTacgTggaccaggagcTggacaTcaaccggcTgTccg |
| | | acTacgacgTggaccacaTcgTgccccagTccTTccTgaaggacgacTccaTcgacaacaaggTgcTgacccggTccgacaagaa |
| | | ccggggcaagTccgacaacgTgcccTccgaggaggTggTgaagaagaTgaagaacTacTggcggcagcTgcTgaacgccaagcTg |
| | | aTcacccagcggaagTTcgacaaccTgaccaaggccgagcggggcggccTgTccgagcTggacaaggccggcTTcaTcaagcggc |
| | | agcTggTggagacccggcagaTcaccaagcacgTggcccagaTccTggacTcccggaTgaacaccaagTacgacgagaacgacaa |
| | | gcTgaTccgggaggTgaaggTgaTcacccTgaagTccaagcTggTgTccgacTTccggaaggacTTccagTTcTacaaggTgcgg |
| | | gagaTcaacaacTaccaccacgcccacgacgccTaccTgaacgccgTggTgggcaccgcccTgaTcaagaagTaccccaagcTgg |
| | | agTccgagTTcgTgTacggcgacTacaaggTgTacgacgTgcggaagaTgaTcgccaagTccgagcaggagaTcggcaaggccac |
| | | cgccaagTacTTcTTcTacTccaacaTcaTgaacTTcTTcaagaccgagaTcacccTggccaacggcgagaTccggaagcggccc |
| | | cTgaTcgagaccaacggcgagaccggcgagaTcgTgTgggacaagggccgggacTTcgccaccgTgcggaaggTgcTgTccaTgc |
| | | cccaggTgaacaTcgTgaagaagaccgaggTgcagaccggcggcTTcTccaaggagTccaTccTgcccaagcggaacTccgacaa |
| | | gcTgaTcgcccggaagaaggacTgggaccccaagaagTacggcggcTTcgacTcccccaccgTggccTacTccgTgcTggTggTg |
| | | gccaaggTggagaagggcaagTccaagaagcTgaagTccgTgaaggagcTgcTgggcaTcaccaTcaTggagcggTccTccTTcg |
| | | agaagaaccccaTcgacTTccTggaggccaagggcTacaaggaggTgaagaaggaccTgaTcaTcaagcTgcccaagTacTcccT |
| | | gTTcgagcTggagaacggccggaagcggaTgcTggccTccgccggcgagcTgcagaagggcaacgagcTggcccTgcccTccaag |
| | | TacgTgaacTTccTgTaccTggccTcccacTacgagaagcTgaagggcTcccccgaggacaacgagcagaagcagcTgTTcgTgg |
| | | agcagcacaagcacTaccTggacgagaTcaTcgagcagaTcTccgagTTcTccaagcgggTgaTccTggccgacgccaaccTgga |
| | | caaggTgcTgTccgccTacaacaagcaccgggacaagcccaTccgggagcaggccgagaacaTcaTccaccTgTTcacccTgacc |
| | | aaccTgggcgcccccgccgccTTcaagTacTTcgacaccaccaTcgaccggaagcggTacaccTccaccaaggaggTgcTggacg |
| | | ccacccTgaTccaccagTccaTcaccggccTgTacgagacccggaTcgaccTgTcccagcTgggcggcgacggcggcggcTcccc |
| | | caagaagaagcggaaggTgTgA |
| Open reading frame | 807 | AUGGGACCGAAGAAGAAGAGAAAGGUCGGAGGAGGAAGCACAAACCUGUCGGACAUCAUCGAAAAGGAAACAGGAAAGCAGCUGG |
| for UGI | | UCAUCCAGGAAUCGAUCCUGAUGCUGCCGGAAGAAGUCGAAGAAGUCAUCGGAAACAAGCCGGAAUCGGACAUCCUGGUCCACAC |
| | | AGCAUACGACGAAUCGACAGACGAAAACGUCAUGCUGCUGACAUCGGACGCACCGGAAUACAAGCCGUGGGCACUGGUCAUCCAG |
| | | GACUCGAACGGAGAAAACAAGAUCAAGAUGCUGUGA |
| Open reading frame | 808 | AUGACCAACCUGUCCGACAUCAUCGAGAAGGAGACCGGCAAGCAGCUGGUGAUCCAGGAGUCCAUCCUGAUGCUGCCCGAGGAGG |
| for UGI | | UGGAGGAGGUGAUCGGCAACAAGCCCGAGUCCGACAUCCUGGUGCACACCGCCUACGACGAGUCCACCGACGAGAACGUGAUGCU |
| | | GCUGACCUCCGACGCCCCCGAGUACAAGCCCUGGGCCCUGGUGAUCCAGGACUCCAACGGCGAGAACAAGAUCAAGAUGCUGUCC |
| | | GGCGGCUCCAAGCGGACCGCCGACGGCUCCGAGUUCGAGUCCCCCAAGAAGAAGCGGAAGGUGGAGUGA |
| Amino acid sequence | 809 | MDKKYSIGLDIGTNSVGWAVITDEYKVPSKKFKVLGNTDRHSIKKNLIGALLFDSGETAEATRLKRTARRRYTRRKNRICYLQEI |
| for Cas9 encoded by | | FSNEMAKVDDSFFHRLEESFLVEEDKKHERHPIFGNIVDEVAYHEKYPTIYHLRKKLVDSTDKADLRLIYLALAHMIKFRGHFLI |
| SEQ ID Nos. 801-802 | | EGDLNPDNSDVDKLFIQLVQTYNQLFEENPINASGVDAKAILSARLSKSRRLENLIAQLPGEKKNGLFGNLIALSLGLTPNFKSN |
| | | FDLAEDAKLQLSKDTYDDDLDNLLAQIGDQYADLELAAKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEELLVKLNREDLLRKQRTFDNGSIPHQIHLGELHAILRR |
| | | QEDFYPFLKDNREKIEKILTFRIPYYVGPLARGNSRFAWMTRKSEETITPWNFEEVVDKGASAQSFIERMTNFDKNLPNEKVLPK |
| | | HSLLYEYFTVYNELTKVKYVTEGMRKPAFLSGEQKKAIVDLLFKTNRKVTVKQLKEDYFKKIECFDSVEISGVEDRFNASLGTYH |
| | | DLLKIIKDKDFLDNEENEDILEDIVLTLTLFEDREMIEERLKTYAHLFDDKVMKQLKRRRYTGWGRLSRKLINGIRDKQSGKTIL |
| | | DFLKSDGFANRNFMQLIHDDSLTFKEDIQKAQVSGQGDSLHEHIANLAGSPAIKKGILQTVKVVDELVKVMGRHKPENIVIEMAR |
| | | ENQTTQKGQKNSRERMKRIEEGIKELGSQILKEHPVENTQLQNEKLYLYYLQNGRDMYVDQELDINRLSDYDVDHIVPQSFLKDD |
| | | SIDNKVLTRSDKNRGKSDNVPSEEVVKKMKNYWRQLLNAKLITQRKFDNLTKAERGGLSELDKAGFIKRQLVETRQITKHVAQIL |
| | | DSRMNTKYDENDKLIREVKVITLKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIMERSSFEKNPIDELEAKGYKEV |
| | | KKDLIIKLPKYSLFELENGRKRMLASAGELQKGNELALPSKYVNFLYLASHYEKLKGSPEDNEQKQLFVEQHKHYLDEIIEQISE |
| | | FSKRVILADANLDKVLSAYNKHRDKPIREQAENIIHLFTLTNLGAPAAFKYEDTTIDRKRYTSTKEVLDATLIHQSITGLYETRI |
| | | DLSQLGGDGGGSPKKKRKV |
| Amino acid sequence | 810 | MDKKYSIGLDIGTNSVGWAVITDEYKVPSKKFKVLGNTDRHSIKKNLIGALLFDSGETAEATRLKRTARRRYTRRKNRICYLQEI |
| for Cas9 with Hibit | | FSNEMAKVDDSFFHRLEESFLVEEDKKHERHPIFGNIVDEVAYHEKYPTIYHLRKKLVDSTDKADLRLIYLALAHMIKFRGHFLI |
| tag | | EGDLNPDNSDVDKLFIQLVQTYNQLFEENPINASGVDAKAILSARLSKSRRLENLIAQLPGEKKNGLFGNLIALSLGLTPNFKSN |
| | | FDLAEDAKLQLSKDTYDDDLDNLLAQIGDQYADLFLAAKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEELLVKLNREDLLRKQRTFDNGSIPHQIHLGELHAILRR |
| | | QEDFYPFLKDNREKIEKILTFRIPYYVGPLARGNSRFAWMTRKSEETITPWNFEEVVDKGASAQSFIERMTNFDKNLPNEKVLPK |
| | | HSLLYEYFTVYNELTKVKYVTEGMRKPAFLSGEQKKAIVDLLFKTNRKVTVKQLKEDYFKKIECFDSVEISGVEDRFNASLGTYH |
| | | DLLKIIKDKDFLDNEENEDILEDIVLTLTLFEDREMIEERLKTYAHLFDDKVMKQLKRRRYTGWGRLSRKLINGIRDKQSGKTIL |
| | | DFLKSDGFANRNFMQLIHDDSLTFKEDIQKAQVSGQGDSLHEHIANLAGSPAIKKGILQTVKVVDELVKVMGRHKPENIVIEMAR |
| | | ENQTTQKGQKNSRERMKRIEEGIKELGSQILKEHPVENTQLQNEKLYLYYLQNGRDMYVDQELDINRLSDYDVDHIVPQSFLKDD |
| | | SIDNKVLTRSDKNRGKSDNVPSEEVVKKMKNYWRQLLNAKLITQRKFDNLTKAERGGLSELDKAGFIKRQLVETRQITKHVAQIL |
| | | DSRMNTKYDENDKLIREVKVITLKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIMERSSFEKNPIDFLEAKGYKEV |
| | | KKDLIIKLPKYSLFELENGRKRMLASAGELQKGNELALPSKYVNFLYLASHYEKLKGSPEDNEQKQLFVEQHKHYLDEIIEQISE |
| | | FSKRVILADANLDKVLSAYNKHRDKPIREQAENIIHLFTLTNLGAPAAFKYEDTTIDRKRYTSTKEVLDATLIHQSITGLYETRI |
| | | DLSQLGGDGGGSPKKKRKVSESATPESVSGWRLFKKIS |
| Amino acid sequence | 811 | MEASPASGPRHLMDPHIFTSNFNNGIGRHKTYLCYEVERLDNGTSVKMDQHRGELHNQAKNLLCGFYGRHAELRFLDLVPSLQLD |
| for BC22n | | PAQIYRVTWFISWSPCFSWGCAGEVRAFLQENTHVRLRIFAARIYDYDPLYKEALQMLRDAGAQVSIMTYDEFKHCWDTFVDHQG |
| | | CPFQPWDGLDEHSQALSGRLRAILQNQGNSGSETPGTSESATPESDKKYSIGLAIGTNSVGWAVITDEYKVPSKKFKVLGNTDRH |
| | | SIKKNLIGALLFDSGETAEATRLKRTARRRYTRRKNRICYLQEIFSNEMAKVDDSFFHRLEESFLVEEDKKHERHPIFGNIVDEV |
| | | AYHEKYPTIYHLRKKLVDSTDKADLRLIYLALAHMIKERGHFLIEGDLNPDNSDVDKLFIQLVQTYNQLFEENPINASGVDAKAI |
| | | LSARLSKSRRLENLIAQLPGEKKNGLFGNLIALSLGLTPNEKSNEDLAEDAKLQLSKDTYDDDLDNLLAQIGDQYADLFLAAKNL |
| | | SDAILLSDILRVNTEITKAPLSASMIKRYDEHHQDLTLLKALVRQQLPEKYKEIFFDQSKNGYAGYIDGGASQEEFYKFIKPILE |
| | | KMDGTEELLVKLNREDLLRKQRTFDNGSIPHQIHLGELHAILRRQEDFYPFLKDNREKIEKILTFRIPYYVGPLARGNSRFAWMT |
| | | RKSEETITPWNFEEVVDKGASAQSFIERMTNEDKNLPNEKVLPKHSLLYEYFTVYNELTKVKYVTEGMRKPAFLSGEQKKAIVDL |
| | | LFKTNRKVTVKQLKEDYFKKIECFDSVEISGVEDRFNASLGTYHDLLKIIKDKDFLDNEENEDILEDIVLTLTLFEDREMIEERL |
| | | KTYAHLFDDKVMKQLKRRRYTGWGRLSRKLINGIRDKQSGKTILDFLKSDGFANRNEMQLIHDDSLTFKEDIQKAQVSGQGDSLH |
| | | EHIANLAGSPAIKKGILQTVKVVDELVKVMGRHKPENIVIEMARENQTTQKGQKNSRERMKRIEEGIKELGSQILKEHPVENTQL |
| | | QNEKLYLYYLQNGRDMYVDQELDINRLSDYDVDHIVPQSFLKDDSIDNKVLTRSDKNRGKSDNVPSEEVVKKMKNYWRQLLNAKL |
| | | ITQRKFDNLTKAERGGLSELDKAGFIKRQLVETRQITKHVAQILDSRMNTKYDENDKLIREVKVITLKSKLVSDFRKDFQFYKVR |
| | | EINNYHHAHDAYLNAVVGTALIKKYPKLESEFVYGDYKVYDVRKMIAKSEQEIGKATAKYFFYSNIMNFFKTEITLANGEIRKRP |
| | | LIETNGETGEIVWDKGRDFATVRKVLSMPQVNIVKKTEVQTGGFSKESILPKRNSDKLIARKKDWDPKKYGGEDSPTVAYSVLVV |
| | | AKVEKGKSKKLKSVKELLGITIMERSSFEKNPIDFLEAKGYKEVKKDLIIKLPKYSLFELENGRKRMLASAGELQKGNELALPSK |
| | | YVNFLYLASHYEKLKGSPEDNEQKQLFVEQHKHYLDEIIEQISEFSKRVILADANLDKVLSAYNKHRDKPIREQAENIIHLFTLT |
| | | NLGAPAAFKYFDTTIDRKRYTSTKEVLDATLIHQSITGLYETRIDLSQLGGDGGGSPKKKRKV* |
| Amino acid sequence | 812 | MEASPASGPRHLMDPHIFTSNFNNGIGRHKTYLCYEVERLDNGTSVKMDQHRGFLHNQAKNLLCGFYGRHAELRFLDLVPSLQLD |
| for BC22n with Hibit | | PAQIYRVTWFISWSPCFSWGCAGEVRAFLQENTHVRLRIFAARIYDYDPLYKEALQMLRDAGAQVSIMTYDEFKHCWDTFVDHQG |
| tag | | CPFQPWDGLDEHSQALSGRLRAILQNQGNSGSETPGTSESATPESDKKYSIGLAIGTNSVGWAVITDEYKVPSKKEKVLGNTDRH |
| | | SIKKNLIGALLFDSGETAEATRLKRTARRRYTRRKNRICYLQEIFSNEMAKVDDSFFHRLEESELVEEDKKHERHPIFGNIVDEV |
| | | AYHEKYPTIYHLRKKLVDSTDKADLRLIYLALAHMIKFRGHFLIEGDLNPDNSDVDKLFIQLVQTYNQLFEENPINASGVDAKAI |
| | | LSARLSKSRRLENLIAQLPGEKKNGLFGNLIALSLGLTPNFKSNEDLAEDAKLQLSKDTYDDDLDNLLAQIGDQYADLFLAAKNL |
| | | SDAILLSDILRVNTEITKAPLSASMIKRYDEHHQDLTLLKALVRQQLPEKYKEIFFDQSKNGYAGYIDGGASQEEFYKFIKPILE |
| | | KMDGTEELLVKLNREDLLRKQRTEDNGSIPHQIHLGELHAILRRQEDFYPELKDNREKIEKILTFRIPYYVGPLARGNSRFAWMT |
| | | RKSEETITPWNFEEVVDKGASAQSFIERMTNEDKNLPNEKVLPKHSLLYEYFTVYNELTKVKYVTEGMRKPAFLSGEQKKAIVDL |
| | | LFKTNRKVTVKQLKEDYFKKIECFDSVEISGVEDRFNASLGTYHDLLKIIKDKDFLDNEENEDILEDIVLTLTLFEDREMIEERL |
| | | KTYAHLEDDKVMKQLKRRRYTGWGRLSRKLINGIRDKQSGKTILDFLKSDGFANRNFMQLIHDDSLTFKEDIQKAQVSGQGDSLH |
| | | EHIANLAGSPAIKKGILQTVKVVDELVKVMGRHKPENIVIEMARENQTTQKGQKNSRERMKRIEEGIKELGSQILKEHPVENTQL |
| | | QNEKLYLYYLQNGRDMYVDQELDINRLSDYDVDHIVPQSFLKDDSIDNKVLTRSDKNRGKSDNVPSEEVVKKMKNYWRQLLNAKL |
| | | ITQRKFDNLTKAERGGLSELDKAGFIKRQLVETRQITKHVAQILDSRMNTKYDENDKLIREVKVITLKSKLVSDFRKDFQFYKVR |
| | | EINNYHHAHDAYLNAVVGTALIKKYPKLESEFVYGDYKVYDVRKMIAKSEQEIGKATAKYFFYSNIMNFFKTEITLANGEIRKRP |
| | | LIETNGETGEIVWDKGRDFATVRKVLSMPQVNIVKKTEVQTGGFSKESILPKRNSDKLIARKKDWDPKKYGGEDSPTVAYSVLVV |
| | | AKVEKGKSKKLKSVKELLGITIMERSSFEKNPIDFLEAKGYKEVKKDLIIKLPKYSLFELENGRKRMLASAGELQKGNELALPSK |
| | | YVNFLYLASHYEKLKGSPEDNEQKQLFVEQHKHYLDEIIEQISEFSKRVILADANLDKVLSAYNKHRDKPIREQAENIIHLETLT |
| | | NLGAPAAFKYFDTTIDRKRYTSTKEVLDATLIHQSITGLYETRIDLSQLGGDGGGSPKKKRKVSESATPESVSGWRLFKKIS |
| | 813 | Not used |
| Amino acid sequence | 814 | MTNLSDIIEKETGKQLVIQESILMLPEEVEEVIGNKPESDILVHTAYDESTDENVMLLTSDAPEYKPWALVIQDSNGENKIKMLS |
| for UGI | | GGSKRTADGSEFESPKKKRKVE |
| | 815-816 | Not used |
| Open reading frame | 817 | AUGGACAAGAAGUACAGCAUCGGACUGGACAUCGGAACAAACAGCGUCGGAUGGGCAGUCAUCACAGACGAAUACAAGGUCCCGA |
| for Cas9 | | GCAAGAAGUUCAAGGUCCUGGGAAACACAGACAGACACAGCAUCAAGAAGAACCUGAUCGGAGCACUGCUGUUCGACAGCGGAGA |
| | | AACAGCAGAAGCAACAAGACUGAAGAGAACAGCAAGAAGAAGAUACACAAGAAGAAAGAACAGAAUCUGCUACCUGCAGGAAAUC |
| | | UUCAGCAACGAAAUGGCAAAGGUCGACGACAGCUUCUUCCACAGACUGGAAGAAAGCUUCCUGGUCGAAGAAGACAAGAAGCACG |
| | | AAAGACACCCGAUCUUCGGAAACAUCGUCGACGAAGUCGCAUACCACGAAAAGUACCCGACAAUCUACCACCUGAGAAAGAAGCU |
| | | GGUCGACAGCACAGACAAGGCAGACCUGAGACUGAUCUACCUGGCACUGGCACACAUGAUCAAGUUCAGAGGACACUUCCUGAUC |
| | | GAAGGAGACCUGAACCCGGACAACAGCGACGUCGACAAGCUGUUCAUCCAGCUGGUCCAGACAUACAACCAGCUGUUCGAAGAAA |
| | | ACCCGAUCAACGCAAGCGGAGUCGACGCAAAGGCAAUCCUGAGCGCAAGACUGAGCAAGAGCAGAAGACUGGAAAACCUGAUCGC |
| | | ACAGCUGCCGGGAGAAAAGAAGAACGGACUGUUCGGAAACCUGAUCGCACUGAGCCUGGGACUGACACCGAACUUCAAGAGCAAC |
| | | UUCGACCUGGCAGAAGACGCAAAGCUGCAGCUGAGCAAGGACACAUACGACGACGACCUGGACAACCUGCUGGCACAGAUCGGAG |
| | | ACCAGUACGCAGACCUGUUCCUGGCAGCAAAGAACCUGAGCGACGCAAUCCUGCUGAGCGACAUCCUGAGAGUCAACACAGAAAU |
| | | CACAAAGGCACCGCUGAGCGCAAGCAUGAUCAAGAGAUACGACGAACACCACCAGGACCUGACACUGCUGAAGGCACUGGUCAGA |
| | | CAGCAGCUGCCGGAAAAGUACAAGGAAAUCUUCUUCGACCAGAGCAAGAACGGAUACGCAGGAUACAUCGACGGAGGAGCAAGCC |
| | | AGGAAGAAUUCUACAAGUUCAUCAAGCCGAUCCUGGAAAAGAUGGACGGAACAGAAGAACUGCUGGUCAAGCUGAACAGAGAAGA |
| | | CCUGCUGAGAAAGCAGAGAACAUUCGACAACGGAAGCAUCCCGCACCAGAUCCACCUGGGAGAACUGCACGCAAUCCUGAGAAGA |
| | | CAGGAAGACUUCUACCCGUUCCUGAAGGACAACAGAGAAAAGAUCGAAAAGAUCCUGACAUUCAGAAUCCCGUACUACGUCGGAC |
| | | CGCUGGCAAGAGGAAACAGCAGAUUCGCAUGGAUGACAAGAAAGAGCGAAGAAACAAUCACACCGUGGAACUUCGAAGAAGUCGU |
| | | CGACAAGGGAGCAAGCGCACAGAGCUUCAUCGAAAGAAUGACAAACUUCGACAAGAACCUGCCGAACGAAAAGGUCCUGCCGAAG |
| | | CACAGCCUGCUGUACGAAUACUUCACAGUCUACAACGAACUGACAAAGGUCAAGUACGUCACAGAAGGAAUGAGAAAGCCGGCAU |
| | | UCCUGAGCGGAGAACAGAAGAAGGCAAUCGUCGACCUGCUGUUCAAGACAAACAGAAAGGUCACAGUCAAGCAGCUGAAGGAAGA |
| | | CUACUUCAAGAAGAUCGAAUGCUUCGACAGCGUCGAAAUCAGCGGAGUCGAAGACAGAUUCAACGCAAGCCUGGGAACAUACCAC |
| | | GACCUGCUGAAGAUCAUCAAGGACAAGGACUUCCUGGACAACGAAGAAAACGAAGACAUCCUGGAAGACAUCGUCCUGACACUGA |
| | | CACUGUUCGAAGACAGAGAAAUGAUCGAAGAAAGACUGAAGACAUACGCACACCUGUUCGACGACAAGGUCAUGAAGCAGCUGAA |
| | | GAGAAGAAGAUACACAGGAUGGGGAAGACUGAGCAGAAAGCUGAUCAACGGAAUCAGAGACAAGCAGAGCGGAAAGACAAUCCUG |
| | | GACUUCCUGAAGAGCGACGGAUUCGCAAACAGAAACUUCAUGCAGCUGAUCCACGACGACAGCCUGACAUUCAAGGAAGACAUCC |
| | | AGAAGGCACAGGUCAGCGGACAGGGAGACAGCCUGCACGAACACAUCGCAAACCUGGCAGGAAGCCCGGCAAUCAAGAAGGGAAU |
| | | CCUGCAGACAGUCAAGGUCGUCGACGAACUGGUCAAGGUCAUGGGAAGACACAAGCCGGAAAACAUCGUCAUCGAAAUGGCAAGA |
| | | GAAAACCAGACAACACAGAAGGGACAGAAGAACAGCAGAGAAAGAAUGAAGAGAAUCGAAGAAGGAAUCAAGGAACUGGGAAGCC |
| | | AGAUCCUGAAGGAACACCCGGUCGAAAACACACAGCUGCAGAACGAAAAGCUGUACCUGUACUACCUGCAGAACGGAAGAGACAU |
| | | GUACGUCGACCAGGAACUGGACAUCAACAGACUGAGCGACUACGACGUCGACCACAUCGUCCCGCAGAGCUUCCUGAAGGACGAC |
| | | AGCAUCGACAACAAGGUCCUGACAAGAAGCGACAAGAACAGAGGAAAGAGCGACAACGUCCCGAGCGAAGAAGUCGUCAAGAAGA |
| | | UGAAGAACUACUGGAGACAGCUGCUGAACGCAAAGCUGAUCACACAGAGAAAGUUCGACAACCUGACAAAGGCAGAGAGAGGAGG |
| | | ACUGAGCGAACUGGACAAGGCAGGAUUCAUCAAGAGACAGCUGGUCGAAACAAGACAGAUCACAAAGCACGUCGCACAGAUCCUG |
| | | GACAGCAGAAUGAACACAAAGUACGACGAAAACGACAAGCUGAUCAGAGAAGUCAAGGUCAUCACACUGAAGAGCAAGCUG |
| | | GUCAGCGACUUCAGAAAGGACUUCCAGUUCUACAAGGUCAGAGAAAUCAACAACUACCACCACGCACACGACGCAUACCUGAACG |
| | | CAGUCGUCGGAACAGCACUGAUCAAGAAGUACCCGAAGCUGGAAAGCGAAUUCGUCUACGGAGACUACAAGGUCUACGACGUCAG |
| | | AAAGAUGAUCGCAAAGAGCGAACAGGAAAUCGGAAAGGCAACAGCAAAGUACUUCUUCUACAGCAACAUCAUGAACUUCUUCAAG |
| | | ACAGAAAUCACACUGGCAAACGGAGAAAUCAGAAAGAGACCGCUGAUCGAAACAAACGGAGAAACAGGAGAAAUCGUCUGGGACA |
| | | AGGGAAGAGACUUCGCAACAGUCAGAAAGGUCCUGAGCAUGCCGCAGGUCAACAUCGUCAAGAAGACAGAAGUCCAGACAGGAGG |
| | | AUUCAGCAAGGAAAGCAUCCUGCCGAAGAGAAACAGCGACAAGCUGAUCGCAAGAAAGAAGGACUGGGACCCGAAGAAGUACGGA |
| | | GGAUUCGACAGCCCGACAGUCGCAUACAGCGUCCUGGUCGUCGCAAAGGUCGAAAAGGGAAAGAGCAAGAAGCUGAAGAGCGUCA |
| | | AGGAACUGCUGGGAAUCACAAUCAUGGAAAGAAGCAGCUUCGAAAAGAACCCGAUCGACUUCCUGGAAGCAAAGGGAUACAAGGA |
| | | AGUCAAGAAGGACCUGAUCAUCAAGCUGCCGAAGUACAGCCUGUUCGAACUGGAAAACGGAAGAAAGAGAAUGCUGGCAAGCGCA |
| | | GGAGAACUGCAGAAGGGAAACGAACUGGCACUGCCGAGCAAGUACGUCAACUUCCUGUACCUGGCAAGCCACUACGAAAAGCUGA |
| | | AGGGAAGCCCGGAAGACAACGAACAGAAGCAGCUGUUCGUCGAACAGCACAAGCACUACCUGGACGAAAUCAUCGAACAGAUCAG |
| | | CGAAUUCAGCAAGAGAGUCAUCCUGGCAGACGCAAACCUGGACAAGGUCCUGAGCGCAUACAACAAGCACAGAGACAAGCCGAUC |
| | | AGAGAACAGGCAGAAAACAUCAUCCACCUGUUCACACUGACAAACCUGGGAGCACCGGCAGCAUUCAAGUACUUCGACACAACAA |
| | | UCGACAGAAAGAGAUACACAAGCACAAAGGAAGUCCUGGACGCAACACUGAUCCACCAGAGCAUCACAGGACUGUACGAAACAAG |
| | | AAUCGACCUGAGCCAGCUGGGAGGAGACGGAGGAGGAAGCCCGAAGAAGAAGAGAAAGGUCUAG |
| Open reading frame | 818 | AUGGAAGCAAGCCCGGCAAGCGGACCGAGACACCUGAUGGACCCGCACAUCUUCACAAGCAACUUCAACAACGGAAUCGGAAGAC |
| for BC22 | | ACAAGACAUACCUGUGCUACGAAGUCGAAAGACUGGACAACGGAACAAGCGUCAAGAUGGACCAGCACAGAGGAUUCCUGCACAA |
| | | CCAGGCAAAGAACCUGCUGUGCGGAUUCUACGGAAGACACGCAGAACUGAGAUUCCUGGACCUGGUCCCGAGCCUGCAGCUGGAC |
| | | CCGGCACAGAUCUACAGAGUCACAUGGUUCAUCAGCUGGAGCCCGUGCUUCAGCUGGGGAUGCGCAGGAGAAGUCAGAGCAUUUC |
| | | UGCAGGAAAACACACACGUCAGACUGAGAAUCUUCGCAGCAAGAAUCUAC |
| | | GACUACGACCCGCUGUACAAGGAAGCACUGCAGAUGCUGAGAGACGCAGGAGCACAGGUCAGCAUCAUGACAUACGACGAAUUCA |
| | | AGCACUGCUGGGACACAUUCGUCGACCACCAGGGAUGCCCGUUCCAGCCGUGGGACGGACUGGACGAACACAGCCAGGCACUGAG |
| | | CGGAAGACUGAGAGCAAUCCUGCAGAACCAGGGAAACAGCGGAAGCGAAACACCGGGAACAAGCGAAAGCGCAACACCGGAAAGC |
| | | GACAAGAAGUACAGCAUCGGACUGGCCAUCGGAACAAACAGCGUCGGAUGGGCAGUCAUCACAGACGAAUACAAGGUCCCGAGCA |
| | | AGAAGUUCAAGGUCCUGGGAAACACAGACAGACACAGCAUCAAGAAGAACCUGAUCGGAGCACUGCUGUUCGACAGCGGAGAAAC |
| | | AGCAGAAGCAACAAGACUGAAGAGAACAGCAAGAAGAAGAUACACAAGAAGAAAGAACAGAAUCUGCUACCUGCAGGAAAUCUUC |
| | | AGCAACGAAAUGGCAAAGGUCGACGACAGCUUCUUCCACAGACUGGAAGAAAGCUUCCUGGUCGAAGAAGACAAGAAGCACGAAA |
| | | GACACCCGAUCUUCGGAAACAUCGUCGACGAAGUCGCAUACCACGAAAAGUACCCGACAAUCUACCACCUGAGAAAGAAGCUGGU |
| | | CGACAGCACAGACAAGGCAGACCUGAGACUGAUCUACCUGGCACUGGCACACAUGAUCAAGUUCAGAGGACACUUCCUGAUCGAA |
| | | GGAGACCUGAACCCGGACAACAGCGACGUCGACAAGCUGUUCAUCCAGCUGGUCCAGACAUACAACCAGCUGUUCGAAGAAAACC |
| | | CGAUCAACGCAAGCGGAGUCGACGCAAAGGCAAUCCUGAGCGCAAGACUGAGCAAGAGCAGAAGACUGGAAAACCUGAUCGCACA |
| | | GCUGCCGGGAGAAAAGAAGAACGGACUGUUCGGAAACCUGAUCGCACUGAGCCUGGGACUGACACCGAACUUCAAGAGCAACUUC |
| | | GACCUGGCAGAAGACGCAAAGCUGCAGCUGAGCAAGGACACAUACGACGACGACCUGGACAACCUGCUGGCACAGAUCGGAGACC |
| | | AGUACGCAGACCUGUUCCUGGCAGCAAAGAACCUGAGCGACGCAAUCCUGCUGAGCGACAUCCUGAGAGUCAACACAGAAAUCAC |
| | | AAAGGCACCGCUGAGCGCAAGCAUGAUCAAGAGAUACGACGAACACCACCAGGACCUGACACUGCUGAAGGCACUGGUCAGACAG |
| | | CAGCUGCCGGAAAAGUACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGCUGAACAGAGAAGACCU |
| | | GCUGAGAAAGCAGAGAACAUUCGACAACGGAAGCAUCCCGCACCAGAUCCACCUGGGAGAACUGCACGCAAUCCUGAGAAGACAG |
| | | GAAGACUUCUACCCGUUCCUGAAGGACAACAGAGAAAAGAUCGAAAAGAUCCUGACAUUCAGAAUCCCGUACUACGUCGGACCGC |
| | | UGGCAAGAGGAAACAGCAGAUUCGCAUGGAUGACAAGAAAGAGCGAAGAAACAAUCACACCGUGGAACUUCGAAGAAGUCGUCGA |
| | | CAAGGGAGCAAGCGCACAGAGCUUCAUCGAAAGAAUGACAAACUUCGACAAGAACCUGCCGAACGAAAAGGUCCUGCCGAAGCAC |
| | | AGCCUGCUGUACGAAUACUUCACAGUCUACAACGAACUGACAAAGGUCAAGUACGUCACAGAAGGAAUGAGAAAGCCGGCAUUCC |
| | | UGAGCGGAGAACAGAAGAAGGCAAUCGUCGACCUGCUGUUCAAGACAAACAGAAAGGUCACAGUCAAGCAGCUGAAGGAAGACUA |
| | | CUUCAAGAAGAUCGAAUGCUUCGACAGCGUCGAAAUCAGCGGAGUCGAAGACAGAUUCAACGCAAGCCUGGGAACAUACCACGAC |
| | | CUGCUGAAGAUCAUCAAGGACAAGGACUUCCUGGACAACGAAGAAAACGAAGACAUCCUGGAAGACAUCGUCCUGACACUGACAC |
| | | UGUUCGAAGACAGAGAAAUGAUCGAAGAAAGACUGAAGACAUACGCACACCUGUUCGACGACAAGGUCAUGAAGCAGCUGAAGAG |
| | | AAGAAGAUACACAGGAUGGGGAAGACUGAGCAGAAAGCUGAUCAACGGAAUCAGAGACAAGCAGAGCGGAAAGACAAUCCUGGAC |
| | | UUCCUGAAGAGCGACGGAUUCGCAAACAGAAACUUCAUGCAGCUGAUCCACGACGACAGCCUGACAUUCAAGGAAGACAUCCAGA |
| | | AGGCACAGGUCAGCGGACAGGGAGACAGCCUGCACGAACACAUCGCAAACCUGGCAGGAAGCCCGGCAAUCAAGAAGGGAAUCCU |
| | | GCAGACAGUCAAGGUCGUCGACGAACUGGUCAAGGUCAUGGGAAGACACAAGCCGGAAAACAUCGUCAUCGAAAUGGCAAGAGAA |
| | | AACCAGACAACACAGAAGGGACAGAAGAACAGCAGAGAAAGAAUGAAGAGAAUCGAAGAAGGAAUCAAGGAACUGGGAAGCCAGA |
| | | UCCUGAAGGAACACCCGGUCGAAAACACACAGCUGCAGAACGAAAAGCUGUACCUGUACUACCUGCAGAACGGAAGAGACAUGUA |
| | | CGUCGACCAGGAACUGGACAUCAACAGACUGAGCGACUACGACGUCGACCACAUCGUCCCGCAGAGCUUCCUGAAGGACGACAGC |
| | | AUC |
| | | GACAACAAGGUCCUGACAAGAAGCGACAAGAACAGAGGAAAGAGCGACAACGUCCCGAGCGAAGAAGUCGUCAAGAAGAUGAAGA |
| | | ACUACUGGAGACAGCUGCUGAACGCAAAGCUGAUCACACAGAGAAAGUUCGACAACCUGACAAAGGCAGAGAGAGGAGGACUGAG |
| | | CGAACUGGACAAGGCAGGAUUCAUCAAGAGACAGCUGGUCGAAACAAGACAGAUCACAAAGCACGUCGCACAGAUCCUGGACAGC |
| | | AGAAUGAACACAAAGUACGACGAAAACGACAAGCUGAUCAGAGAAGUCAAGGUCAUCACACUGAAGAGCAAGCUGGUCAGCGACU |
| | | UCAGAAAGGACUUCCAGUUCUACAAGGUCAGAGAAAUCAACAACUACCACCACGCACACGACGCAUACCUGAACGCAGUCGUCGG |
| | | AACAGCACUGAUCAAGAAGUACCCGAAGCUGGAAAGCGAAUUCGUCUACGGAGACUACAAGGUCUACGACGUCAGAAAGAUGAUC |
| | | GCAAAGAGCGAACAGGAAAUCGGAAAGGCAACAGCAAAGUACUUCUUCUACAGCAACAUCAUGAACUUCUUCAAGACAGAAAUCA |
| | | CACUGGCAAACGGAGAAAUCAGAAAGAGACCGCUGAUCGAAACAAACGGAGAAACAGGAGAAAUCGUCUGGGACAAGGGAAGAGA |
| | | CUUCGCAACAGUCAGAAAGGUCCUGAGCAUGCCGCAGGUCAACAUCGUCAAGAAGACAGAAGUCCAGACAGGAGGAUUCAGCAAG |
| | | GAAAGCAUCCUGCCGAAGAGAAACAGCGACAAGCUGAUCGCAAGAAAGAAGGACUGGGACCCGAAGAAGUACGGAGGAUUCGACA |
| | | GCCCGACAGUCGCAUACAGCGUCCUGGUCGUCGCAAAGGUCGAAAAGGGAAAGAGCAAGAAGCUGAAGAGCGUCAAGGAACUGCU |
| | | GGGAAUCACAAUCAUGGAAAGAAGCAGCUUCGAAAAGAACCCGAUCGACUUCCUGGAAGCAAAGGGAUACAAGGAAGUCAAGAAG |
| | | GACCUGAUCAUCAAGCUGCCGAAGUACAGCCUGUUCGAACUGGAAAACGGAAGAAAGAGAAUGCUGGCAAGCGCAGGAGAACUGC |
| | | AGAAGGGAAACGAACUGGCACUGCCGAGCAAGUACGUCAACUUCCUGUACCUGGCAAGCCACUACGAAAAGCUGAAGGGAAGCCC |
| | | GGAAGACAACGAACAGAAGCAGCUGUUCGUCGAACAGCACAAGCACUACCUGGACGAAAUCAUCGAACAGAUCAGCGAAUUCAGC |
| | | AAGAGAGUCAUCCUGGCAGACGCAAACCUGGACAAGGUCCUGAGCGCAUACAACAAGCACAGAGACAAGCCGAUCAGAGAACAGG |
| | | CAGAAAACAUCAUCCACCUGUUCACACUGACAAACCUGGGAGCACCGGCAGCAUUCAAGUACUUCGACACAACAAUCGACAGAAA |
| | | GAGAUACACAAGCACAAAGGAAGUCCUGGACGCAACACUGAUCCACCAGAGCAUCACAGGACUGUACGAAACAAGAAUCGAUCUG |
| | | AGCCAGCUGGGAGGAGACAGCGGAGGAAGCACAAACCUGAGCGACAUCAUCGAAAAGGAAACAGGAAAGCAGCUGGUCAUCCAGG |
| | | AAAGCAUCCUGAUGCUGCCGGAAGAAGUCGAAGAAGUCAUCGGAAACAAGCCGGAAAGCGACAUCCUGGUCCACACAGCAUACGA |
| | | CGAAAGCACAGACGAAAACGUCAUGCUGCUGACAAGCGACGCACCGGAAUACAAGCCGUGGGCACUGGUCAUCCAGGACAGCAAC |
| | | GGAGAAAACAAGAUCAAGAUGCUGAGCGGAGGAAGCCCGAAGAAGAAGAGAAAGGUCUAA |
| Open reading frame | 819 | AUGGGACCGAAGAAGAAGAGAAAGGUCGGAGGAGGAAGCACAAACCUGUCGGACAUCAUCGAAAAGGAAACAGGAAAGCAGCUGG |
| for UGI | | UCAUCCAGGAAUCGAUCCUGAUGCUGCCGGAAGAAGUCGAAGAAGUCAUCGGAAACAAGCCGGAAUCGGACAUCCUGGUCCACAC |
| | | AGCAUACGACGAAUCGACAGACGAAAACGUCAUGCUGCUGACAUCGGACGCACCGGAAUACAAGCCGUGGGCACUGGUCAUCCAG |
| | | GACUCGAACGGAGAAAACAAGAUCAAGAUGCUGUGA |
| mRNA encoding UGI | 821 | GGGAAGCUCAGAAUAAACGCUCAACUUUGGCCGGAUCUGCCACCAUGACCAACCUGUCCGACAUCAUCGAGAAGGAGACCGGCAA |
| | | GCAGCUGGUGAUCCAGGAGUCCAUCCUGAUGCUGCCCGAGGAGGUGGAGGAGGUGAUCGGCAACAAGCCCGAGUCCGACAUCCUG |
| | | GUGCACACCGCCUACGACGAGUCCACCGACGAGAACGUGAUGCUGCUGACCUCCGACGCCCCCGAGUACAAGCCCUGGGCCCUGG |
| | | UGAUCCAGGACUCCAACGGCGAGAACAAGAUCAAGAUGCUGUCCGGCGGCUCCAAGCGGACCGCCGACGGCUCCGAGUUCGAGUC |
| | | CCCCAAGAAGAAGCGGAAGGUGGAGUGAUAGCUAGCACCAGCCUCAAGAACACCCGAAUGGAGUCUCUAAGCUACAUAAUACCAA |
| | | CUUACACUUUACAAAAUGUUGUCCCCCAAAAUGUAGCCAUUCGUAUCUGCUCCUAAUAAAAAGAAAGUUUCUUCACAUUCUCUCG |
| | | AGAAAAAAAAAAAAUGGAAAAAAAAAAAACGGAAAAAAAAAAAAGGUAAAAAAAAAAAAUAUAAAAAAAAAAAACAUAAAAAAAA |
| | | AAAACGAAAAAAAAAAAACGUAAAAAAAAAAAACUCAAAAAAAAAAAAGAUAAAAAAAAAAAACCUAAAAAAAAAAAAUGUAAAA |
| | | AAAAAAAAGGGAAAAAAAAAAAACGCAAAAAAAAAAAACACAAAAAAAAAAAAUGCAAAAAAAAAAAAUCGAAAAAAAAAAAAUC |
| | | UAAAAAAAAAAAACGAAAAAAAAAAAACCCAAAAAAAAAAAAGACAAAAAAAAAAAAUAGAAAAAAAAAAAAGUUAAAAAAAAAA |
| | | AACUGAAAAAAAAAAAAUUUAAAAAAAAAAAAUCUAG |
| mRNA encoding | 822 | GGGAAGCUCAGAAUAAACGCUCAACUUUGGCCGGAUCUGCCACCAUGGACGGCUCCGGCGGCGGCUCCCCCAAGAAGAAGCGGAA |
| Nme2Cas9 base editor | | GGUGGAGGACAAGCGGCCCGCCGCCACCAAGAAGGCCGGCCAGGCCAAGAAGAAGAAGGGCGGCUCCGGCGGCGGCGAGGCCUCC |
| | | CCCGCCUCCGGCCCCCGGCACCUGAUGGACCCCCACAUCUUCACCUCCAACUUCAACAACGGCAUCGGCCGGCACAAGACCUACC |
| | | UGUGCUACGAGGUGGAGCGGCUGGACAACGGCACCUCCGUGAAGAUGGACCAGCACCGGGGCUUCCUGCACAACCAGGCCAAGAA |
| | | CCUGCUGUGCGGCUUCUACGGCCGGCACGCCGAGCUGCGGUUCCUGGACCUGGUGCCCUCCCUGCAGCUGGACCCCGCCCAGAUC |
| | | UACCGGGUGACCUGGUUCAUCUCCUGGUCCCCCUGCUUCUCCUGGGGCUGCGCCGGCGAGGUGCGGGCCUUCCUGCAGGAGAACA |
| | | CCCACGUGCGGCUGCGGAUCUUCGCCGCCCGGAUCUACGACUACGACCCCCUGUACAAGGAGGCCCUGCAGAUGCUGCGGGACGC |
| | | CGGCGCCCAGGUGUCCAUCAUGACCUACGACGAGUUCAAGCACUGCUGGGACACCUUCGUGGACCACCAGGGCUGCCCCUUCCAG |
| | | CCCUGGGACGGCCUGGACGAGCACUCCCAGGCCCUGUCCGGCCGGCUGCGGGCCAUCCUGCAGAACCAGGGCAACUCCGGCUCCG |
| | | AGACCCCCGGCACCUCCGAGUCCGCCACCCCCGAGUCCGCAGCGUUCAAACCAAAUCCCAUCAACUACAUCCUGGGCCUGGCCAU |
| | | CGGCAUCGCCUCCGUGGGCUGGGCCAUGGUGGAGAUCGACGAGGAGGAGAACCCCAUCCGGCUGAUCGACCUGGGCGUGCGGGUG |
| | | UUCGAGCGGGCCGAGGUGCCCAAGACCGGCGACUCCCUGGCCAUGGCCCGGCGGCUGGCCCGGUCCGUGCGGCGGCUGACCCGGC |
| | | GGCGGGCCCACCGGCUGCUGCGGGCCCGGCGGCUGCUGAAGCGGGAGGGCGUGCUGCAGGCCGCCGACUUCGACGAGAACGGCCU |
| | | GAUCAAGUCCCUGCCCAACACCCCCUGGCAGCUGCGGGCCGCCGCCCUGGACCGGAAGCUGACCCCCCUGGAGUGGUCCGCCGUG |
| | | CUGCUGCACCUGAUCAAGCACCGGGGCUACCUGUCCCAGCGGAAGAACGAGGGCGAGACCGCCGACAAGGAGCUGGGCGCCCUGC |
| | | UGAAGGGCGUGGCCAACAACGCCCACGCCCUGCAGACCGGCGACUUCCGGACCCCCGCCGAGCUGGCCCUGAACAAGUUCGAGAA |
| | | GGAGUCCGGCCACAUCCGGAACCAGCGGGGCGACUACUCCCACACCUUCUCCCGGAAGGACCUGCAGGCCGAGCUGAUCCUGCUG |
| | | UUCGAGAAGCAGAAGGAGUUCGGCAACCCCCACGUGUCCGGCGGCCUGAAGGAGGGCAUCGAGACCCUGCUGAUGACCCAGCGGC |
| | | CCGCCCUGUCCGGCGACGCCGUGCAGAAGAUGCUGGGCCACUGCACCUUCGAGCCCGCCGAGCCCAAGGCCGCCAAGAACACCUA |
| | | CACCGCCGAGCGGUUCAUCUGGCUGACCAAGCUGAACAACCUGCGGAUCCUGGAGCAGGGCUCCGAGCGGCCCCUGACCGACACC |
| | | GAGCGGGCCACCCUGAUGGACGAGCCCUACCGGAAGUCCAAGCUGACCUACGCCCAGGCCCGGAAGCUGCUGGGCCUGGAGGACA |
| | | CCGCCUUCUUCAAGGGCCUGCGGUACGGCAAGGACAACGCCGAGGCCUCCACCCUGAUGGAGAUGAAGGCCUACCACGCCAUCUC |
| | | CCGGGCCCUGGAGAAGGAGGGCCUGAAGGACAAGAAGUCCCCCCUGAACCUGUCCUCCGAGCUGCAGGACGAGAUCGGCACCGCC |
| | | UUCUCCCUGUUCAAGACCGACGAGGACAUCACCGGCCGGCUGAAGGACCGGGUGCAGCCCGAGAUCCUGGAGGCCCUGCUGAAGC |
| | | ACAUCUCCUUCGACAAGUUCGUGCAGAUCUCCCUGAAGGCCCUGCGGCGGAUCGUGCCCCUGAUGGAGCAGGGCAAGCGGUACGA |
| | | CGAGGCCUGCGCCGAGAUCUACGGCGACCACUACGGCAAGAAGAACACCGAGGAGAAGAUCUACCUGCCCCCCAUCCCCGCCGAC |
| | | GAGAUCCGGAACCCCGUGGUGCUGCGGGCCCUGUCCCAGGCCCGGAAGGUGAUCAACGGCGUGGUGCGGCGGUACGGCUCCCCCG |
| | | CCCGGAUCCACAUCGAGACCGCCCGGGAGGUGGGCAAGUCCUUCAAGGACCGGAAGGAGAUCGAGAAGCGGCAGGAGGAGAACCG |
| | | GAAGGACCGGGAGAAGGCCGCCGCCAAGUUCCGGGAGUACUUCCCCAACUUCGUGGGCGAGCCCAAGUCCAAGGACAUCCUGAAG |
| | | CUGCGGCUGUACGAGCAGCAGCACGGCAAGUGCCUGUACUCCGGCAAGGAGAUCAACCUGGUGCGGCUGAACGAGAAGGGCUACG |
| | | UGGAGAUCGACCACGCCCUGCCCUUCUCCCGGACCUGGGACGACUCCUUCAACAACAAGGUGCUGGUGCUGGGCUCCGAGAACCA |
| | | GAACAAGGGCAACCAGACCCCCUACGAGUACUUCAACGGCAAGGACAACUCCCGGGAGUGGCAGGAGUUCAAGGCCCGGGUGGAG |
| | | ACCUCCCGGUUCCCCCGGUCCAAGAAGCAGCGGAUCCUGCUGCAGAAGUUCGACGAGGACGGCUUCAAGGAGUGCAACCUGAACG |
| | | ACACCCGGUACGUGAACCGCUUCCUGUGCCAGUUCGUGGCCGACCACAUCCUGCUGACCGGCAAGGGCAAGCGGCGGGUGUUCGC |
| | | CUCCAACGGCCAGAUCACCAACCUGCUGCGGGGCUUCUGGGGCCUGCGGAAGGUGCGGGCCGAGAACGACCGGCACCACGCCCUG |
| | | GACGCCGUGGUGGUGGCCUGCUCCACCGUGGCCAUGCAGCAGAAGAUCACCCGGUUCGUGCGGUACAAGGAGAUGAACGCCUUCG |
| | | ACGGCAAGACCAUCGACAAGGAGACCGGCAAGGUGCUGCACCAGAAGACCCACUUCCCCCAGCCCUGGGAGUUCUUCGCCCAGGA |
| | | GGUGAUGAUCCGGGUGUUCGGCAAGCCCGACGGCAAGCCCGAGUUCGAGGAGGCCGACACCCCCGAGAAGCUGCGGACCCUGCUG |
| | | GCCGAGAAGCUGUCCUCCCGGCCCGAGGCCGUGCACGAGUACGUGACCCCCCUGUUCGUGUCCCGGGCCCCCAACCGGAAGAUGU |
| | | CCGGCGCCCACAAGGACACCCUGCGGUCCGCCAAGCGGUUCGUGAAGCACAACGAGAAGAUCUCCGUGAAGCGGGUGUGGCUGAC |
| | | CGAGAUCAAGCUGGCCGACCUGGAGAACAUGGUGAACUACAAGAACGGCCGGGAGAUCGAGCUGUACGAGGCCCUGAAGGCCCGG |
| | | CUGGAGGCCUACGGCGGCAACGCCAAGCAGGCCUUCGACCCCAAGGACAACCCCUUCUACAAGAAGGGCGGCCAGCUGGUGAAGG |
| | | CCGUGCGGGUGGAGAAGACCCAGGAGUCCGGCGUGCUGCUGAACAAGAAGAACGCCUACACCAUCGCCGACAACGGCGACAUGGU |
| | | GCGGGUGGACGUGUUCUGCAAGGUGGACAAGAAGGGCAAGAACCAGUACUUCAUCGUGCCCAUCUACGCCUGGCAGGUGGCCGAG |
| | | AACAUCCUGCCCGACAUCGACUGCAAGGGCUACCGGAUCGACGACUCCUACACCUUCUGCUUCUCCCUGCACAAGUACGACCUGA |
| | | UCGCCUUCCAGAAGGACGAGAAGUCCAAGGUGGAGUUCGCCUACUACAUCAACUGCGACUCCUCCAACGGCCGGUUCUACCUGGC |
| | | CUGGCACGACAAGGGCUCCAAGGAGCAGCAGUUCCGGAUCUCCACCCAGAACCUGGUGCUGAUCCAGAAGUACCAGGUGAACGAG |
| | | CUGGGCAAGGAGAUCCGGCCCUGCCGGCUGAAGAAGCGGCCCCCCGUGCGGUAGUGACUAGCACCAGCCUCAAGAACACCCGAAU |
| | | GGAGUCUCUAAGCUACAUAAUACCAACUUACACUUUACAAAAUGUUGUCCCCCAAAAUGUAGCCAUUCGUAUCUGCUCCUAAUAA |
| | | AAAGAAAGUUUCUUCACAUUCUCUCGAGAAAAAAAAAAAAUGGAAAAAAAAAAAACGGAAAAAAAAAAAGGUAAAAAAAAAAAAU |
| | | AUAAAAAAAAAAACAUAAAAAAAAAAAACGAAAAAAAAAAAACGUAAAAAAAAAAAACUCAAAAAAAAAAAGAUAAAAAAAAAAA |
| | | ACCUAAAAAAAAAAAAUGUAAAAAAAAAAAAGGGAAAAAAAAAAACGCAAAAAAAAAAAACACAAAAAAAAAAAAUGCAAAAAAA |
| | | AAAAAUCGAAAAAAAAAAAAUCUAAAAAAAAAAAACGAAAAAAAAAAAACCCAAAAAAAAAAAAGACAAAAAAAAAAAAUAGAAA |
| | | AAAAAAAAGUUAAAAAAAAAAAACUGAAAAAAAAAAAAUUUAAAAAAAAAAAAUCUAG |
| Open reading frame | 823 | ATGGCAGCATTCAAGCCGAACTCGATCAACTACATCCTGGGACTGGACATCGGAATCGCATCGGTCGGATGGGCAATGGTCGAAA |
| for NmelCas9 | | TCGACGAAGAAGAAAACCCGATCAGACTGATCGACCTGGGAGTCAGAGTCTTCGAAAGAGCAGAAGTCCCGAAGACAGGAGACTC |
| | | GCTGGCAATGGCAAGAAGACTGGCAAGATCGGTCAGAAGACTGACAAGAAGAAGAGCACACAGACTGCTGAGAACAAGAAGACTG |
| | | CTGAAGAGAGAAGGAGTCCTGCAGGCAGCAAACTTCGACGAAAACGGACTGATCAAGTCGCTGCCGAACACACCGTGGCAGCTGA |
| | | GAGCAGCAGCACTGGACAGAAAGCTGACACCGCTGGAATGGTCGGCAGTCCTGCTGCACCTGATCAAGCACAGAGGATACCTGTC |
| | | GCAGAGAAAGAACGAAGGAGAAACAGCAGACAAGGAACTGGGAGCACTGCTGAAGGGAGTCGCAGGAAACGCACACGCACTGCAG |
| | | ACAGGAGACTTCAGAACACCGGCAGAACTGGCACTGAACAAGTTCGAAAAGGAATCGGGACACATCAGAAACCAGAGATCGGACT |
| | | ACTCGCACACATTCTCGAGAAAGGACCTGCAGGCAGAACTGATCCTGCTGTTCGAAAAGCAGAAGGAATTCGGAAACCCGCACGT |
| | | CTCGGGAGGACTGAAGGAAGGAATCGAAACACTGCTGATGACACAGAGACCGGCACTGTCGGGAGACGCAGTCCAGAAGATGCTG |
| | | GGACACTGCACATTCGAACCGGCAGAACCGAAGGCAGCAAAGAACACATACACAGCAGAAAGATTCATCTGGCTGACAAAGCTGA |
| | | ACAACCTGAGAATCCTGGAACAGGGATCGGAAAGACCGCTGACAGACACAGAAAGAGCAACACTGATGGACGAACCGTACAGAAA |
| | | GTCGAAGCTGACATACGCACAGGCAAGAAAGCTGCTGGGACTGGAAGACACAGCATTCTTCAAGGGACTGAGATACGGAAAGGAC |
| | | AACGCAGAAGCATCGACACTGATGGAAATGAAGGCATACCACGCAATCTCGAGAGCACTGGAAAAGGAAGGACTGAAGGACAAGA |
| | | AGTCGCCGCTGAACCTGTCGCCGGAACTGCAGGACGAAATCGGAACAGCATTCTCGCTGTTCAAGACAGACGAAGACATCACAGG |
| | | AAGACTGAAGGACAGAATCCAGCCGGAAATCCTGGAAGCACTGCTGAAGCACATCTCGTTCGACAAGTTCGTCCAGATCTCGCTG |
| | | AAGGCACTGAGAAGAATCGTCCCGCTGATGGAACAGGGAAAGAGATACGACGAAGCATGCGCAGAAATCTACGGAGACCACTACG |
| | | GAAAGAAGAACACAGAAGAAAAGATCTACCTGCCGCCGATCCCGGCAGACGAAATCAGAAACCCGGTCGTCCTGAGAGCACTGTC |
| | | GCAGGCAAGAAAGGTCATCAACGGAGTCGTCAGAAGATACGGATCGCCGGCAAGAATCCACATCGAAACAGCAAGAGAAGTCGGA |
| | | AAGTCGTTCAAGGACAGAAAGGAAATCGAAAAGAGACAGGAAGAAAACAGAAAGGACAGAGAAAAGGCAGCAGCAAAGTTCAGAG |
| | | AATACTTCCCGAACTTCGTCGGAGAACCGAAGTCGAAGGACATCCTGAAGCTGAGACTGTACGAACAGCAGCACGGAAAGTGCCT |
| | | GTACTCGGGAAAGGAAATCAACCTGGGAAGACTGAACGAAAAGGGATACGTCGAAATCGACCACGCACTGCCGTTCTCGAGAACA |
| | | TGGGACGACTCGTTCAACAACAAGGTCCTGGTCCTGGGATCGGAAAACCAGAACAAGGGAAACCAGACACCGTACGAATACTTCA |
| | | ACGGAAAGGACAACTCGAGAGAATGGCAGGAATTCAAGGCAAGAGTCGAAACATCGAGATTCCCGAGATCGAAGAAGCAGAGAAT |
| | | CCTGCTGCAGAAGTTCGACGAAGACGGATTCAAGGAAAGAAACCTGAACGACACAAGATACGTCAACAGATTCCTGTGCCAGTTC |
| | | GTCGCAGACAGAATGAGACTGACAGGAAAGGGAAAGAAGAGAGTCTTCGCATCGAACGGACAGATCACAAACCTGCTGAGAGGAT |
| | | TCTGGGGACTGAGAAAGGTCAGAGCAGAAAACGACAGACACCACGCACTGGACGCAGTCGTCGTCGCATGCTCGACAGTCGCAAT |
| | | GCAGCAGAAGATCACAAGATTCGTCAGATACAAGGAAATGAACGCATTCGACGGAAAGACAATCGACAAGGAAACAGGAGAAGTC |
| | | CTGCACCAGAAGACACACTTCCCGCAGCCGTGGGAATTCTTCGCACAGGAAGTCATGATCAGAGTCTTCGGAAAGCCGGACGGAA |
| | | AGCCGGAATTCGAAGAAGCAGACACACTGGAAAAGCTGAGAACACTGCTGGCAGAAAAGCTGTCGTCGAGACCGGAAGCAGTCCA |
| | | CGAATACGTCACACCGCTGTTCGTCTCGAGAGCACCGAACAGAAAGATGTCGGGACAGGGACACATGGAAACAGTCAAGTCGGCA |
| | | AAGAGACTGGACGAAGGAGTCTCGGTCCTGAGAGTCCCGCTGACACAGCTGAAGCTGAAGGACCTGGAAAAGATGGTCAACAGAG |
| | | AAAGAGAACCGAAGCTGTACGAAGCACTGAAGGCAAGACTGGAAGCACACAAGGACGACCCGGCAAAGGCATTCGCAGAACCGTT |
| | | CTACAAGTACGACAAGGCAGGAAACAGAACACAGCAGGTCAAGGCAGTCAGAGTCGAACAGGTCCAGAAGACAGGAGTCTGGGTC |
| | | AGAAACCACAACGGAATCGCAGACAACGCAACAATGGTCAGAGTAGACGTCTTCGAAAAGGGAGACAAGTACTACCTGGTCCCGA |
| | | TCTACTCGTGGCAGGTCGCAAAGGGAATCCTGCCGGACAGAGCAGTCGTCCAGGGAAAGGACGAAGAAGACTGGCAGCTGATCGA |
| | | CGACTCGTTCAACTTCAAGTTCTCGCTGCACCCGAACGACCTGGTCGAAGTCATCACAAAGAAGGCAAGAATGTTCGGATACTTC |
| | | GCATCGTGCCACAGAGGAACAGGAAACATCAACATCAGAATCCACGACCTGGACCACAAGATCGGAAAGAACGGAATCCTGGAAG |
| | | GAATCGGAGTCAAGACAGCACTGTCGTTCCAGAAGTACCAGATCGACGAACTGGGAAAGGAAATCAGACCGTGCAGACTGAAGAA |
| | | GAGACCGCCGGTCAGATCCGGAAAGAGAACAGCAGACGGATCGGAATTCGAATCGCCGAAGAAGAAGAGAAAGGTCGAATGA |
| Open reading frame | 824 | ATGACCAACCTGTCCGACATCATCGAGAAGGAGACCGGCAAGCAGCTGGTGATCCAGGAGTCCATCCTGATGCTGCCCGAGGAGG |
| for UGI encoded by | | TGGAGGAGGTGATCGGCAACAAGCCCGAGTCCGACATCCTGGTGCACACCGCCTACGACGAGTCCACCGACGAGAACGTGATGCT |
| SEQ NO: 821 | | GCTGACCTCCGACGCCCCCGAGTACAAGCCCTGGGCCCTGGTGATCCAGGACTCCAACGGCGAGAACAAGATCAAGATGCTGTCC |
| | | GGCGGCTCCAAGCGGACCGCCGACGGCTCCGAGTTCGAGTCCCCCAAGAAGAAGCGGAAGGTGGAGTGATAG |
| mRNA encoding Nme2 | 825 | GGGAAGCUCAGAAUAAACGCUCAACUUUGGCCGGAUCUGCCACCAUGGACGGCUCCGGCGGCGGCUCCCCCAAGAAGAAGCGGAA |
| Cas9 | | GGUGGAGGACAAGCGGCCCGCCGCCACCAAGAAGGCCGGCCAGGCCAAGAAGAAGAAGGGCGGCUCCGGCGGCGGCGCCGCCUUC |
| | | AAGCCCAACCCCAUCAACUACAUCCUGGGCCUGGACAUCGGCAUCGCCUCCGUGGGCUGGGCCAUGGUGGAGAUCGACGAGGAGG |
| | | AGAACCCCAUCCGGCUGAUCGACCUGGGCGUGCGGGUGUUCGAGCGGGCCGAGGUGCCCAAGACCGGCGACUCCCUGGCCAUGGC |
| | | CCGGCGGCUGGCCCGGUCCGUGCGGCGGCUGACCCGGCGGCGGGCCCACCGGCUGCUGCGGGCCCGGCGGCUGCUGAAGCGGGAG |
| | | GGCGUGCUGCAGGCCGCCGACUUCGACGAGAACGGCCUGAUCAAGUCCCUGCCCAACACCCCCUGGCAGCUGCGGGCCGCCGCCC |
| | | UGGACCGGAAGCUGACCCCCCUGGAGUGGUCCGCCGUGCUGCUGCACCUGAUCAAGCACCGGGGCUACCUGUCCCAGCGGAAGAA |
| | | CGAGGGCGAGACCGCCGACAAGGAGCUGGGCGCCCUGCUGAAGGGCGUGGCCAACAACGCCCACGCCCUGCAGACCGGCGACUUC |
| | | CGGACCCCCGCCGAGCUGGCCCUGAACAAGUUCGAGAAGGAGUCCGGCCACAUCCGGAACCAGCGGGGCGACUACUCCCACACCU |
| | | UCUCCCGGAAGGACCUGCAGGCCGAGCUGAUCCUGCUGUUCGAGAAGCAGAAGGAGUUCGGCAACCCCCACGUGUCCGGCGGCCU |
| | | GAAGGAGGGCAUCGAGACCCUGCUGAUGACCCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCAUCUGGCUGACCAAGCUGAACAACCUGCGGA |
| | | UCCUGGAGCAGGGCUCCGAGCGGCCCCUGACCGACACCGAGCGGGCCACCCUGAUGGACGAGCCCUACCGGAAGUCCAAGCUGAC |
| | | CUACGCCCAGGCCCGGAAGCUGCUGGGCCUGGAGGACACCGCCUUCUUCAAGGGCCUGCGGUACGGCAAGGACAACGCCGAGGCC |
| | | UCCACCCUGAUGGAGAUGAAGGCCUACCACGCCAUCUCCCGGGCCCUGGAGAAGGAGGGCCUGAAGGACAAGAAGUCCCCCCUGA |
| | | ACCUGUCCUCCGAGCUGCAGGACGAGAUCGGCACCGCCUUCUCCCUGUUCAAGACCGACGAGGACAUCACCGGCCGGCUGAAGGA |
| | | CCGGGUGCAGCCCGAGAUCCUGGAGGCCCUGCUGAAGCACAUCUCCUUCGACAAGUUCGUGCAGAUCUCCCUGAAGGCCCUGCGG |
| | | CGGAUCGUGCCCCUGAUGGAGCAGGGCAAGCGGUACGACGAGGCCUGCGCCGAGAUCUACGGCGACCACUACGGCAAGAAGAACA |
| | | CCGAGGAGAAGAUCUACCUGCCCCCCAUCCCCGCCGACGAGAUCCGGAACCCCGUGGUGCUGCGGGCCCUGUCCCAGGCCCGGAA |
| | | GGUGAUCAACGGCGUGGUGCGGCGGUACGGCUCCCCCGCCCGGAUCCACAUCGAGACCGCCCGGGAGGUGGGCAAGUCCUUCAAG |
| | | GACCGGAAGGAGAUCGAGAAGCGGCAGGAGGAGAACCGGAAGGACCGGGAGAAGGCCGCCGCCAAGUUCCGGGAGUACUUCCCCA |
| | | ACUUCGUGGGCGAGCCCAAGUCCAAGGACAUCCUGAAGCUGCGGCUGUACGAGCAGCAGCACGGCAAGUGCCUGUACUCCGGCAA |
| | | GGAGAUCAACCUGGUGCGGCUGAACGAGAAGGGCUACGUGGAGAUCGACCACGCCCUGCCCUUCUCCCGGACCUGGGACGACUCC |
| | | UUCAACAACAAGGUGCUGGUGCUGGGCUCCGAGAACCAGAACAAGGGCAACCAGACCCCCUACGAGUACUUCAACGGCAAGGACA |
| | | ACUCCCGGGAGUGGCAGGAGUUCAAGGCCCGGGUGGAGACCUCCCGGUUCCCCCGGUCCAAGAAGCAGCGGAUCCUGCUGCAGAA |
| | | GUUCGACGAGGACGGCUUCAAGGAGUGCAACCUGAACGACACCCGGUACGUGAACCGGUUCCUGUGCCAGUUCGUGGCCGACCAC |
| | | AUCCUGCUGACCGGCAAGGGCAAGCGGCGGGUGUUCGCCUCCAACGGCCAGAUCACCAACCUGCUGCGGGGCUUCUGGGGCCUGC |
| | | GGAAGGUGCGGGCCGAGAACGACCGGCACCACGCCCUGGACGCCGUGGUGGUGGCCUGCUCCACCGUGGCCAUGCAGCAGAAGAU |
| | | CACCCGGUUCGUGCGGUACAAGGAGAUGAACGCCUUCGACGGCAAGACCAUCGACAAGGAGACCGGCAAGGUGCUGCACCAGAAG |
| | | ACCCACUUCCCCCAGCCCUGGGAGUUCUUCGCCCAGGAGGUGAUGAUCCGGGUGUUCGGCAAGCCCGACGGCAAGCCCGAGUUCG |
| | | AGGAGGCCGACACCCCCGAGAAGCUGCGGACCCUGCUGGCCGAGAAGCUGUCCUCCCGGCCCGAGGCCGUGCACGAGUACGUGAC |
| | | CCCCCUGUUCGUGUCCCGGGCCCCCAACCGGAAGAUGUCCGGCGCCCACAAGGACACCCUGCGGUCCGCCAAGCGGUUCGUGAAG |
| | | CACAACGAGAAGAUCUCCGUGAAGCGGGUGUGGCUGACCGAGAUCAAGCUGGCCGACCUGGAGAACAUGGUGAACUACAAGAACG |
| | | GCCGGGAGAUCGAGCUGUACGAGGCCCUGAAGGCCCGGCUGGAGGCCUACGGCGGCAACGCCAAGCAGGCCUUCGACCCCAAGGA |
| | | CAACCCCUUCUACAAGAAGGGCGGCCAGCUGGUGAAGGCCGUGCGGGUGGAGAAGACCCAGGAGUCCGGCGUGCUGCUGAACAAG |
| | | AAGAACGCCUACACCAUCGCCGACAACGGCGACAUGGUGCGGGUGGACGUGUUCUGCAAGGUGGACAAGAAGGGCAAGAACCAGU |
| | | ACUUCAUCGUGCCCAUCUACGCCUGGCAGGUGGCCGAGAACAUCCUGCCCGACAUCGACUGCAAGGGCUACCGGAUCGACGACUC |
| | | CUACACCUUCUGCUUCUCCCUGCACAAGUACGACCUGAUCGCCUUCCAGAAGGACGAGAAGUCCAAGGUGGAGUUCGCCUACUAC |
| | | AUCAACUGCGACUCCUCCAACGGCCGGUUCUACCUGGCCUGGCACGACAAGGGCUCCAAGGAGCAGCAGUUCCGGAUCUCCACCC |
| | | AGAACCUGGUGCUGAUCCAGAAGUACCAGGUGAACGAGCUGGGCAAGGAGAUCCGGCCCUGCCGGCUGAAGAAGCGGCCCCCCGU |
| | | GCGGUAGCUAGCACCAGCCUCAAGAACACCCGAAUGGAGUCUCUAAGCUACAUAAUACCAACUUACACUUUACAAAAUGUUGUCC |
| | | CCCAAAAUGUAGCCAUUCGUAUCUGCUCCUAAUAAAAAGAAAGUUUCUUCACAUUCUCUCGAGAAAAAAAAAAAAUGGAAAAAAA |
| | | AAAAACGGAAAAAAAAAAAAGGUAAAAAAAAAAAAUAUAAAAAAAAAAAACAUAAAAAAAAAAAACGAAAAAAAAAAAACGUAAA |
| | | AAAAAAAAACUCAAAAAAAAAAAGAUAAAAAAAAAAAACCUAAAAAAAAAAAAUGUAAAAAAAAAAAAGGGAAAAAAAAAAAACG |
| | | CAAAAAAAAAAAACACAAAAAAAAAAAAUGCAAAAAAAAAAAAUCGAAAAAAAAAAAAUCUAAAAAAAAAAAACGAAAAAAAAAA |
| | | AACCCAAAAAAAAAAAAGACAAAAAAAAAAAAUAGAAAAAAAAAAAGUUAAAAAAAAAAAACUGAAAAAAAAAAAAUUUAAAAAA |
| | | AAAAAAUCUAG |
| mRNA encoding Nme2 | 826 | GGGAAGCUCAGAAUAAACGCUCAACUUUGGCCGGAUCUGCCACCAUGGACGGCUCCGGCGGCGGCUCCCCCAAGAAGAAGCGGAA |
| Cas9 with Hibit tag | | GGUGGAGGACAAGCGGCCCGCCGCCACCAAGAAGGCCGGCCAGGCCAAGAAGAAGAAGGGCGGCUCCGGCGGCGGCGCCGCCUUC |
| | | AAGCCCAACCCCAUCAACUACAUCCUGGGCCUGGACAUCGGCAUCGCCUCCGUGGGCUGGGCCAUGGUGGAGAUCGACGAGGAGG |
| | | AGAACCCCAUCCGGCUGAUCGACCUGGGCGUGCGGGUGUUCGAGCGGGCCGAGGUGCCCAAGACCGGCGACUCCCUGGCCAUGGC |
| | | CCGGCGGCUGGCCCGGUCCGUGCGGCGGCUGACCCGGCGGCGGGCCCACCGGCUGCUGCGGGCCCGGCGGCUGCUGAAGCGGGAG |
| | | GGCGUGCUGCAGGCCGCCGACUUCGACGAGAACGGCCUGAUCAAGUCCCUGCCCAACACCCCCUGGCAGCUGCGGGCCGCCGCCC |
| | | UGGACCGGAAGCUGACCCCCCUGGAGUGGUCCGCCGUGCUGCUGCACCUGAUCAAGCACCGGGGCUACCUGUCCCAGCGGAAGAA |
| | | CGAGGGCGAGACCGCCGACAAGGAGCUGGGCGCCCUGCUGAAGGGCGUGGCCAACAACGCCCACGCCCUGCAGACCGGCGACUUC |
| | | CGGACCCCCGCCGAGCUGGCCCUGAACAAGUUCGAGAAGGAGUCCGGCCACAUCCGGAACCAGCGGGGCGACUACUCCCACACCU |
| | | UCUCCCGGAAGGACCUGCAGGCCGAGCUGAUCCUGCUGUUCGAGAAGCAGAAGGAGUUCGGCAACCCCCACGUGUCCGGCGGCCU |
| | | GAAGGAGGGCAUCGAGACCCUGCUGAUGACCCAGCGGCCCGCCCUGUCCGGCGACGCCGUGCAGAAGAUGCUGGGCCACUGCACC |
| | | UUCGAGCCCGCCGAGCCCAAGGCCGCCAAGAACACCUACACCGCCGAGCGGUUCAUCUGGCUGACCAAGCUGAACAACCUGCGGA |
| | | UCCUGGAGCAGGGCUCCGAGCGGCCCCUGACCGACACCGAGCGGGCCACCCUGAUGGACGAGCCCUACCGGAAGUCCAAGCUGAC |
| | | CUACGCCCAGGCCCGGAAGCUGCUGGGCCUGGAGGACACCGCCUUCUUCAAGGGCCUGCGGUACGGCAAGGACAACGCCGAGGCC |
| | | UCCACCCUGAUGGAGAUGAAGGCCUACCACGCCAUCUCCCGGGCCCUGGAGAAGGAGGGCCUGAAGGACAAGAAGUCCCCCCUGA |
| | | ACCUGUCCUCCGAGCUGCAGGACGAGAUCGGCACCGCCUUCUCCCUGUUCAAGACCGACGAGGACAUCACCGGCCGGCUGAAGGA |
| | | CCGGGUGCAGCCCGAGAUCCUGGAGGCCCUGCUGAAGCACAUCUCCUUCGACAAGUUCGUGCAGAUCUCCCUGAAGGCCCUGCGG |
| | | CGGAUCGUGCCCCUGAUGGAGCAGGGCAAGCGGUACGACGAGGCCUGCGCCGAGAUCUACGGCGACCACUACGGCAAGAAGAACA |
| | | CCGAGGAGAAGAUCUACCUGCCCCCCAUCCCCGCCGACGAGAUCCGGAACCCCGUGGUGCUGCGGGCCCUGUCCCAGGCCCGGAA |
| | | GGUGAUCAACGGCGUGGUGCGGCGGUACGGCUCCCCCGCCCGGAUCCACAUCGAGACCGCCCGGGAGGUGGGCAAGUCCUUCAAG |
| | | GACCGGAAGGAGAUCGAGAAGCGGCAGGAGGAGAACCGGAAGGACCGGGAGAAGGCCGCCGCCAAGUUCCGGGAGUACUUCCCCA |
| | | ACUUCGUGGGCGAGCCCAAGUCCAAGGACAUCCUGAAGCUGCGGCUGUACGAGCAGCAGCACGGCAAGUGCCUGUACUCCGGCAA |
| | | GGAGAUCAACCUGGUGCGGCUGAACGAGAAGGGCUACGUGGAGAUCGACCACGCCCUGCCCUUCUCCCGGACCUGGGACGACUCC |
| | | UUCAACAACAAGGUGCUGGUGCUGGGCUCCGAGAACCAGAACAAGGGCAACCAGACCCCCUACGAGUACUUCAACGGCAAGGACA |
| | | ACUCCCGGGAGUGGCAGGAGUUCAAGGCCCGGGUGGAGACCUCCCGGUUCCCCCGGUCCAAGAAGCAGCGGAUCCUGCUGCAGAA |
| | | GUUCGACGAGGACGGCUUCAAGGAGUGCAACCUGAACGACACCCGGUACGUGAACCGGUUCCUGUGCCAGUUCGUGGCCGACCAC |
| | | AUCCUGCUGACCGGCAAGGGCAAGCGGCGGGUGUUCGCCUCCAACGGCCAGAUCACCAACCUGCUGCGGGGCUUCUGGGGCCUGC |
| | | GGAAGGUGCGGGCCGAGAACGACCGGCACCACGCCCUGGACGCCGUGGUGGUGGCCUGCUCCACCGUGGCCAUGCAGCAGAAGAU |
| | | CACCCGGUUCGUGCGGUACAAGGAGAUGAACGCCUUCGACGGCAAGACCAUCGACAAGGAGACCGGCAAGGUGCUGCACCAGAAG |
| | | ACCCACUUCCCCCAGCCCUGGGAGUUCUUCGCCCAGGAGGUGAUGAUCCGGGUGUUCGGCAAGCCCGACGGCAAGCCCGAGUUCG |
| | | AGGAGGCCGACACCCCCGAGAAGCUGCGGACCCUGCUGGCCGAGAAGCUGUCCUCCCGGCCCGAGGCCGUGCACGAGUACGUGAC |
| | | CCCCCUGUUCGUGUCCCGGGCCCCCAACCGGAAGAUGUCCGGCGCCCACAAGGACACCCUGCGGUCCGCCAAGCGGUUCGUGAAG |
| | | CACAACGAGAAGAUCUCCGUGAAGCGGGUGUGGCUGACCGAGAUCAAGCUGGCCGACCUGGAGAACAUGGUGAACUACAAGAACG |
| | | GCCGGGAGAUCGAGCUGUACGAGGCCCUGAAGGCCCGGCUGGAGGCCUACGGCGGCAACGCCAAGCAGGCCUUCGACCCCAAGGA |
| | | CAACCCCUUCUACAAGAAGGGCGGCCAGCUGGUGAAGGCCGUGCGGGUGGAGAAGACCCAGGAGUCCGGCGUGCUGCUGAACAAG |
| | | AAGAACGCCUACACCAUCGCCGACAACGGCGACAUGGUGCGGGUGGACGUGUUCUGCAAGGUGGACAAGAAGGGCAAGAACCAGU |
| | | ACUUCAUCGUGCCCAUCUACGCCUGGCAGGUGGCCGAGAACAUCCUGCCCGACAUCGACUGCAAGGGCUACCGGAUCGACGACUC |
| | | CUACACCUUCUGCUUCUCCCUGCACAAGUACGACCUGAUCGCCUUCCAGAAGGACGAGAAGUCCAAGGUGGAGUUCGCCUACUAC |
| | | AUCAACUGCGACUCCUCCAACGGCCGGUUCUACCUGGCCUGGCACGACAAGGGCUCCAAGGAGCAGCAGUUCCGGAUCUCCACCC |
| | | AGAACCUGGUGCUGAUCCAGAAGUACCAGGUGAACGAGCUGGGCAAGGAGAUCCGGCCCUGCCGGCUGAAGAAGCGGCCCCCCGU |
| | | GCGGUCCGAGUCCGCCACCCCCGAGUCCGUGUCCGGCUGGCGGCUGUUCAAGAAGAUCUCCUAGCUAGCACCAGCCUCAAGAACA |
| | | CCCGAAUGGAGUCUCUAAGCUACAUAAUACCAACUUACACUUUACAAAAUGUUGUCCCCCAAAAUGUAGCCAUUCGUAUCUGCUC |
| | | CUAAUAAAAAGAAAGUUUCUUCACAUUCUCUCGAGAAAAAAAAAAAAUGGAAAAAAAAAAAACGGAAAAAAAAAAAAGGUAAAAA |
| | | AAAAAAAUAUAAAAAAAAAAAACAUAAAAAAAAAAAACGAAAAAAAAAAAACGUAAAAAAAAAAAACUCAAAAAAAAAAAAGAUA |
| | | AAAAAAAAAAACCUAAAAAAAAAAAAUGUAAAAAAAAAAAAGGGAAAAAAAAAAAACGCAAAAAAAAAAAACACAAAAAAAAAAA |
| | | AUGCAAAAAAAAAAAAUCGAAAAAAAAAAAAUCUAAAAAAAAAAAACGAAAAAAAAAAAACCCAAAAAAAAAAAAGACAAAAAAA |
| | | AAAAAUAGAAAAAAAAAAAAGUUAAAAAAAAAAAACUGAAAAAAAAAAAAUUUAAAAAAAAAAAAUCUAG |
| ORF encoding Spy Cas9 | 827 | ATGGACAAGAAGTACTCCATCGGCCTGGACATCGGCACCAACTCCGTGGGCTGGGCCGTGATCACCGACGAGTACAAGGTGCCCT |
| | | CCAAGAAGTTCAAGGTGCTGGGCAACACCGACCGGCACTCCATCAAGAAGAACCTGATCGGCGCCCTGCTGTTCGACTCCGGCGA |
| | | GACCGCCGAGGCCACCCGGCTGAAGCGGACCGCCCGGCGGCGGTACACCCGGCGGAAGAACCGGATCTGCTACCTGCAGGAGATC |
| | | TTCTCCAACGAGATGGCCAAGGTGGACGACTCCTTCTTCCACCGGCTGGAGGAGTCCTTCCTGGTGGAGGAGGACAAGAAGCACG |
| | | AGCGGCACCCCATCTTCGGCAACATCGTGGACGAGGTGGCCTACCACGAGAAGTACCCCACCATCTACCACCTGCGGAAGAAGCT |
| | | GGTGGACTCCACCGACAAGGCCGACCTGCGGCTGATCTACCTGGCCCTGGCCCACATGATCAAGTTCCGGGGCCACTTCCTGATC |
| | | GAGGGCGACCTGAACCCCGACAACTCCGACGTGGACAAGCTGTTCATCCAGCTGGTGCAGACCTACAACCAGCTGTTCGAGGAGA |
| | | ACCCCATCAACGCCTCCGGCGTGGACGCCAAGGCCATCCTGTCCGCCCGGCTGTCCAAGTCCCGGCGGCTGGAGAACCTGATCGC |
| | | CCAGCTGCCCGGCGAGAAGAAGAACGGCCTGTTCGGCAACCTGATCGCCCTGTCCCTGGGCCTGACCCCCAACTTCAAGTCCAAC |
| | | TTCGACCTGGCCGAGGACGCCAAGCTGCAGCTGTCCAAGGACACCTACGACGACGACCTGGACAACCTGCTGGCCCAGATCGGCG |
| | | ACCAGTACGCCGACCTGTTCCTGGCCGCCAAGAACCTGTCCGACGCCATCCTGCTGTCCGACATCCTGCGGGTGAACACCGAGAT |
| | | CACCAAGGCCCCCCTGTCCGCCTCCATGATCAAGCGGTACGACGAGCACCACCAGGACCTGACCCTGCTGAAGGCCCTGGTGCGG |
| | | CAGCAGCTGCCCGAGAAGTACAAGGAGATCTTCTTCGACCAGTCCAAGAACGGCTACGCCGGCTACATCGACGGCGGCGCCTCCC |
| | | AGGAGGAGTTCTACAAGTTCATCAAGCCCATCCTGGAGAAGATGGACGGCACCGAGGAGCTGCTGGTGAAGCTGAACCGGGAGGA |
| | | CCTGCTGCGGAAGCAGCGGACCTTCGACAACGGCTCCATCCCCCACCAGATCCACCTGGGCGAGCTGCACGCCATCCTGCGGCGG |
| | | CAGGAGGACTTCTACCCCTTCCTGAAGGACAACCGGGAGAAGATCGAGAAGATCCTGACCTTCCGGATCCCCTACTACGTGGGCC |
| | | CCCTGGCCCGGGGCAACTCCCGGTTCGCCTGGATGACCCGGAAGTCCGAGGAGACCATCACCCCCTGGAACTTCGAGGAGGTGGT |
| | | GGACAAGGGCGCCTCCGCCCAGTCCTTCATCGAGCGGATGACCAACTTCGACAAGAACCTGCCCAACGAGAAGGTGCTGCCCAAG |
| | | CACTCCCTGCTGTACGAGTACTTCACCGTGTACAACGAGCTGACCAAGGTGAAGTACGTGACCGAGGGCATGCGGAAGCCCGCCT |
| | | TCCTGTCCGGCGAGCAGAAGAAGGCCATCGTGGACCTGCTGTTCAAGACCAACCGGAAGGTGACCGTGAAGCAGCTGAAGGAGGA |
| | | CTACTTCAAGAAGATCGAGTGCTTCGACTCCGTGGAGATCTCCGGCGTGGAGGACCGGTTCAACGCCTCCCTGGGCACCTACCAC |
| | | GACCTGCTGAAGATCATCAAGGACAAGGACTTCCTGGACAACGAGGAGAACGAGGACATCCTGGAGGACATCGTGCTGACCCTGA |
| | | CCCTGTTCGAGGACCGGGAGATGATCGAGGAGCGGCTGAAGACCTACGCCCACCTGTTCGACGACAAGGTGATGAAGCAGCTGAA |
| | | GCGGCGGCGGTACACCGGCTGGGGCCGGCTGTCCCGGAAGCTGATCAACGGCATCCGGGACAAGCAGTCCGGCAAGACCATCCTG |
| | | GACTTCCTGAAGTCCGACGGCTTCGCCAACCGGAACTTCATGCAGCTGATCCACGACGACTCCCTGACCTTCAAGGAGGACATCC |
| | | AGAAGGCCCAGGTGTCCGGCCAGGGCGACTCCCTGCACGAGCACATCGCCAACCTGGCCGGCTCCCCCGCCATCAAGAAGGGCAT |
| | | CCTGCAGACCGTGAAGGTGGTGGACGAGCTGGTGAAGGTGATGGGCCGGCACAAGCCCGAGAACATCGTGATCGAGATGGCCCGG |
| | | GAGAACCAGACCACCCAGAAGGGCCAGAAGAACTCCCGGGAGCGGATGAAGCGGATCGAGGAGGGCATCAAGGAGCTGGGCTCCC |
| | | AGATCCTGAAGGAGCACCCCGTGGAGAACACCCAGCTGCAGAACGAGAAGCTGTACCTGTACTACCTGCAGAACGGCCGGGACAT |
| | | GTACGTGGACCAGGAGCTGGACATCAACCGGCTGTCCGACTACGACGTGGACCACATCGTGCCCCAGTCCTTCCTGAAGGACGAC |
| | | TCCATCGACAACAAGGTGCTGACCCGGTCCGACAAGAACCGGGGCAAGTCCGACAACGTGCCCTCCGAGGAGGTGGTGAAGAAGA |
| | | TGAAGAACTACTGGCGGCAGCTGCTGAACGCCAAGCTGATCACCCAGCGGAAGTTCGACAACCTGACCAAGGCCGAGCGGGGGG |
| | | CCTGTCCGAGCTGGACAAGGCCGGCTTCATCAAGCGGCAGCTGGTGGAGACCCGGCAGATCACCAAGCACGTGGCCCAGATCCTG |
| | | GACTCCCGGATGAACACCAAGTACGACGAGAACGACAAGCTGATCCGGGAGGTGAAGGTGATCACCCTGAAGTCCAAGCTGGTGT |
| | | CCGACTTCCGGAAGGACTTCCAGTTCTACAAGGTGCGGGAGATCAACAACTACCACCACGCCCACGACGCCTACCTGAACGCCGT |
| | | GGTGGGCACCGCCCTGATCAAGAAGTACCCCAAGCTGGAGTCCGAGTTCGTGTACGGCGACTACAAGGTGTACGACGTGCGGAAG |
| | | ATGATCGCCAAGTCCGAGCAGGAGATCGGCAAGGCCACCGCCAAGTACTTCTTCTACTCCAACATCATGAACTTCTTCAAGACCG |
| | | AGATCACCCTGGCCAACGGCGAGATCCGGAAGCGGCCCCTGATCGAGACCAACGGCGAGACCGGCGAGATCGTGTGGGACAAGGG |
| | | CCGGGACTTCGCCACCGTGCGGAAGGTGCTGTCCATGCCCCAGGTGAACATCGTGAAGAAGACCGAGGTGCAGACCGGCGGCTTC |
| | | TCCAAGGAGTCCATCCTGCCCAAGCGGAACTCCGACAAGCTGATCGCCCGGAAGAAGGACTGGGACCCCAAGAAGTACGGCGGCT |
| | | TCGACTCCCCCACCGTGGCCTACTCCGTGCTGGTGGTGGCCAAGGTGGAGAAGGGCAAGTCCAAGAAGCTGAAGTCCGTGAAGGA |
| | | GCTGCTGGGCATCACCATCATGGAGCGGTCCTCCTTCGAGAAGAACCCCATCGACTTCCTGGAGGCCAAGGGCTACAAGGAGGTG |
| | | AAGAAGGACCTGATCATCAAGCTGCCCAAGTACTCCCTGTTCGAGCTGGAGAACGGCCGGAAGCGGATGCTGGCCTCCGCCGGCG |
| | | AGCTGCAGAAGGGCAACGAGCTGGCCCTGCCCTCCAAGTACGTGAACTTCCTGTACCTGGCCTCCCACTACGAGAAGCTGAAGGG |
| | | CTCCCCCGAGGACAACGAGCAGAAGCAGCTGTTCGTGGAGCAGCACAAGCACTACCTGGACGAGATCATCGAGCAGATCTCCGAG |
| | | TTCTCCAAGCGGGTGATCCTGGCCGACGCCAACCTGGACAAGGTGCTGTCCGCCTACAACAAGCACCGGGACAAGCCCATCCGGG |
| | | AGCAGGCCGAGAACATCATCCACCTGTTCACCCTGACCAACCTGGGCGCCCCCGCCGCCTTCAAGTACTTCGACACCACCATCGA |
| | | CCGGAAGCGGTACACCTCCACCAAGGAGGTGCTGGACGCCACCCTGATCCACCAGTCCATCACCGGCCTGTACGAGACCCGGATC |
| | | GACCTGTCCCAGCTGGGCGGCGACGGCGGCGGCTCCCCCAAGAAGAAGCGGAAGGTGTGA |
| ORF encoding Spy Cas9 | 828 | aagctcagaataaacgctcaactttggccggatctgccaccATGGACGGCTCCGGCGGCGGCTCCCCCAAGAAGAAGCGGAAGGT |
| | | GGAGGACAAGCGGCCCGCCGCCACCAAGAAGGCCGGCCAGGCCAAGAAGAAGAAGGGCGGCTCCGGCGGCGGCGAGGCCTCCCCC |
| | | GCCTCCGGCCCCCGGCACCTGATGGACCCCCACATCTTCACCTCCAACTTCAACAACGGCATCGGCCGGCACAAGACCTACCTGT |
| | | GCTACGAGGTGGAGCGGCTGGACAACGGCACCTCCGTGAAGATGGACCAGCACCGGGGCTTCCTGCACAACCAGGCCAAGAACCT |
| | | GCTGTGCGGCTTCTACGGCCGGCACGCCGAGCTGCGGTTCCTGGACCTGGTGCCCTCCCTGCAGCTGGACCCCGCCCAGATCTAC |
| | | CGGGTGACCTGGTTCATCTCCTGGTCCCCCTGCTTCTCCTGGGGCTGCGCCGGCGAGGTGCGGGCCTTCCTGCAGGAGAACACCC |
| | | ACGTGCGGCTGCGGATCTTCGCCGCCCGGATCTACGACTACGACCCCCTGTACAAGGAGGCCCTGCAGATGCTGCGGGACGCCGG |
| | | CGCCCAGGTGTCCATCATGACCTACGACGAGTTCAAGCACTGCTGGGACACCTTCGTGGACCACCAGGGCTGCCCCTTCCAGCCC |
| | | TGGGACGGCCTGGACGAGCACTCCCAGGCCCTGTCCGGCCGGCTGCGGGCCATCCTGCAGAACCAGGGCAACTCCGGCTCCGAGA |
| | | CCCCCGGCACCTCCGAGTCCGCCACCCCCGAGTCCGCAGCGTTCAAACCAAATcccatcaactacatcctgggcctggccatcgg |
| | | catcgcctccgtgggctgggccatggtggagatcgacgaggaggagaaccccatccggctgatcgacctgggcgtgcgggtgttc |
| | | gagcgggccgaggtgcccaagaccggcgactccctggccatggcccggcggctggcccggtccgtgcggcggctgacccggcggc |
| | | gggcccaccggctgctgcgggcccggcggctgctgaagcgggagggcgtgctgcaggccgccgacttcgacgagaacggcctgat |
| | | caagtccctgcccaacaccccctggcagctgcgggccgccgccctggaccggaagctgacccccctggagtggtccgccgtgctg |
| | | ctgcacctgatcaagcaccggggctacctgtcccagcggaagaacgagggcgagaccgccgacaaggagctgggcgccctgctga |
| | | agggcgtggccaacaacgcccacgccctgcagaccggcgacttccggacccccgccgagctggccctgaacaagttcgagaagga |
| | | gtccggccacatccggaaccagcggggcgactactcccacaccttctcccggaaggacctgcaggccgagctgatcctgctgttc |
| | | gagaagcagaaggagttcggcaacccccacgtgtccggcggcctgaaggagggcatcgagaccctgctgatgacccagcggcccg |
| | | ccctgtccggcgacgccgtgcagaagatgctgggccactgcaccttcgagcccgccgagcccaaggccgccaagaacacctacac |
| | | cgccgagcggttcatctggctgaccaagctgaacaacctgcggatcctggagcagggctccgagcggcccctgaccgacaccgag |
| | | cgggccaccctgatggacgagccctaccggaagtccaagctgacctacgcccaggcccggaagctgctgggcctggaggacaccg |
| | | ccttcttcaagggcctgcggtacggcaaggacaacgccgaggcctccaccctgatggagatgaaggcctaccacgccatctcccg |
| | | ggccctggagaaggagggcctgaaggacaagaagtcccccctgaacctgtcctccgagctgcaggacgagatcggcaccgccttc |
| | | tccctgttcaagaccgacgaggacatcaccggccggctgaaggaccgggtgcagcccgagatcctggaggccctgctgaagcaca |
| | | tctccttcgacaagttcgtgcagatctccctgaaggccctgcggcggatcgtgcccctgatggagcagggcaagcggtacgacga |
| | | ggcctgcgccgagatctacggcgaccactacggcaagaagaacaccgaggagaagatctacctgccccccatccccgccgacgag |
| | | atccggaaccccgtggtgctgcgggccctgtcccaggcccggaaggtgatcaacggcgtggtgcggcggtacggctcccccgccc |
| | | ggatccacatcgagaccgcccgggaggtgggcaagtccttcaaggaccggaaggagatcgagaagcggcaggaggagaaccggaa |
| | | ggaccgggagaaggccgccgccaagttccgggagtacttccccaacttcgtgggcgagcccaagtccaaggacatcctgaagctg |
| | | cggctgtacgagcagcagcacggcaagtgcctgtactccggcaaggagatcaacctggtgcggctgaacgagaagggctacgtgg |
| | | agatcgaccacgccctgcccttctcccggacctgggacgactccttcaacaacaaggtgctggtgctgggctccgagaaccagaa |
| | | caagggcaaccagaccccctacgagtacttcaacggcaaggacaactcccgggagtggcaggagttcaaggcccgggtggagacc |
| | | tcccggttcccccggtccaagaagcagcggatcctgctgcagaagttcgacgaggacggcttcaaggagtgcaacctgaacgaca |
| | | cccggtacgtgaaccgcttcctgtgccagttcgtggccgaccacatcctgctgaccggcaagggcaagcggcgggtgttcgcctc |
| | | caacggccagatcaccaacctgctgcggggcttctggggcctgcggaaggtgcgggccgagaacgaccggcaccacgccctggac |
| | | gccgtggtggtggcctgctccaccgtggccatgcagcagaagatcacccggttcgtgcggtacaaggagatgaacgccttcgacg |
| | | gcaagaccatcgacaaggagaccggcaaggtgctgcaccagaagacccacttcccccagccctgggagttcttcgcccaggaggt |
| | | gatgatccgggtgttcggcaagcccgacggcaagcccgagttcgaggaggccgacacccccgagaagctgcggaccctgctggcc |
| | | gagaagctgtcctcccggcccgaggccgtgcacgagtacgtgacccccctgttcgtgtcccgggcccccaaccggaagatgtccg |
| | | gcgcccacaaggacaccctgcggtccgccaagcggttcgtgaagcacaacgagaagatctccgtgaagcgggtgtggctgaccga |
| | | gatcaagctggccgacctggagaacatggtgaactacaagaacggccgggagatcgagctgtacgaggccctgaaggcccggctg |
| | | gaggcctacggcggcaacgccaagcaggccttcgaccccaaggacaaccccttctacaagaagggcggccagctggtgaaggccg |
| | | tgcgggtggagaagacccaggagtccggcgtgctgctgaacaagaagaacgcctacaccatcgccgacaacggcgacatggtgcg |
| | | ggtggacgtgttctgcaaggtggacaagaagggcaagaaccagtacttcatcgtgcccatctacgcctggcaggtggccgagaac |
| | | atcctgcccgacatcgactgcaagggctaccggatcgacgactcctacaccttctgcttctccctgcacaagtacgacctgatcg |
| | | ccttccagaaggacgagaagtccaaggtggagttcgcctactacatcaactgcgactcctccaacggccggttctacctggcctg |
| | | gcacgacaagggctccaaggagcagcagttccggatctccacccagaacctggtgctgatccagaagtaccaggtgaacgagctg |
| | | ggcaaggagatccggccctgccggctgaagaagcggccccccgtgcggtagTGActagcaccagcctcaagaacacccgaatgga |
| | | gtctctaagctacataataccaacttacactttacaaaatgttgtcccccaaaatgtagccattcgtatctgctcctaataaaaa |
| | | gaaagtttcttcacattct |
| | 829-899 | Not used |
| Exemplary XTEN linker | 900 | SGSETPGTSESATPES |
| sequence | | |
| Exemplary XTEN linker | 901 | SGSETPGTSESA |
| sequence | | |
| Exemplary XTEN linker | 902 | SGSETPGTSESATPEGGSGGS |
| sequence | | |
| Exemplary NLS | 903 | PKKKRKV |
| sequence | | |
| Exemplary NLS | 904 | PKKKRRV |
| sequence | | |
| Exemplary bipartite | 905 | KRPAATKKAGQAKKKK |
| NLS sequence | | |
| exemplary XTEN | 906 | SGSETPGTSESATPES |
| exemplary XTEN | 907 | SGSETPGTSESA |
| exemplary XTEN | 908 | SGSETPGTSESATPEGGSGGS |
| amino acid sequence | 909 | GGS |
| for exemplary linker | | |
| amino acid sequence | 910 | GGGGS |
| for exemplary linker | | |
| amino acid sequence | 911 | EAAAK |
| for exemplary linker | | |
| amino acid sequence | 912 | SEGSA |
| for exemplary linker | | |
| amino acid sequence | 913 | SEGSAGTST |
| for exemplary linker | | |
| amino acid sequence | 914 | GGGGSGGGGS |
| for exemplary linker | | |
| amino acid sequence | 915 | GGGGSEAAAK |
| for exemplary linker | | |
| amino acid sequence | 916 | EAAAKGGGGS |
| for exemplary linker | | |
| amino acid sequence | 917 | EAAAKEAAAK |
| for exemplary linker | | |
| amino acid sequence | 918 | SEGSAGTSTESEGSA |
| for exemplary linker | | |
| amino acid sequence | 919 | GGGGSGGGGSGGGGS |
| for exemplary linker | | |
| amino acid sequence | 920 | GGGGSGGGGSEAAAK |
| for exemplary linker | | |
| amino acid sequence | 921 | GGGGSEAAAKGGGGS |
| for exemplary linker | | |
| amino acid sequence | 922 | EAAAKGGGGSEAAAK |
| for exemplary linker | | |
| amino acid sequence | 923 | EAAAKEAAAKGGGGS |
| for exemplary linker | | |
| amino acid sequence | 924 | SEGSAGTSTESEGSAGTSTE |
| for exemplary linker | | |
| amino acid sequence | 925 | GGGGSGGGGSGGGGSEAAAK |
| for exemplary linker | | |
| amino acid sequence | 926 | GGGGSGGGGSEAAAKGGGGS |
| for exemplary linker | | |
| amino acid sequence | 927 | GGGGSEAAAKGGGGSEAAAK |
| for exemplary linker | | |
| amino acid sequence | 928 | GGGGSEAAAKEAAAKGGGGS |
| for exemplary linker | | |
| amino acid sequence | 929 | GGGGSEAAAKEAAAKEAAAK |
| for exemplary linker | | |
| amino acid sequence | 930 | EAAAKGGGGSGGGGSGGGGS |
| for exemplary linker | | |
| amino acid sequence | 931 | EAAAKGGGGSGGGGSEAAAK |
| for exemplary linker | | |
| amino acid sequence | 932 | EAAAKGGGGSEAAAKGGGGS |
| for exemplary linker | | |
| amino acid sequence | 933 | EAAAKGGGGSEAAAKEAAAK |
| for exemplary linker | | |
| amino acid sequence | 934 | EAAAKEAAAKGGGGSGGGGS |
| for exemplary linker | | |
| amino acid sequence | 935 | EAAAKEAAAKGGGGSEAAAK |
| for exemplary linker | | |
| amino acid sequence | 936 | EAAAKEAAAKEAAAKGGGGS |
| for exemplary linker | | |
| amino acid sequence | 937 | SEGSAGTSTESEGSAGTSTESEGSA |
| for exemplary linker | | |
| amino acid sequence | 938 | GGGGSGGGGSGGGGSGGGGSGGGGS |
| for exemplary linker | | |
| amino acid sequence | 939 | GGGGSGGGGSGGGGSGGGGSEAAAK |
| for exemplary linker | | |
| amino acid sequence | 940 | GGGGGGGGSGGGGSEAAAKGGGGS |
| for exemplary linker | | |
| amino acid sequence | 941 | GGGGSGGGGSGGGGSEAAAKEAAAK |
| for exemplary linker | | |
| amino acid sequence | 942 | GGGGSGGGGSEAAAKGGGGSGGGGS |
| for exemplary linker | | |
| amino acid sequence | 943 | GGGGSGGGGSEAAAKGGGGSEAAAK |
| for exemplary linker | | |
| amino acid sequence | 944 | GGGGSGGGGSEAAAKEAAAKGGGGS |
| for exemplary linker | | |
| amino acid sequence | 945 | GGGGSGGGGSEAAAKEAAAKEAAAK |
| for exemplary linker | | |
| amino acid sequence | 946 | GGGGSEAAAKGGGGSGGGGSGGGGS |
| for exemplary linker | | |
| amino acid sequence | 947 | GGGGSEAAAKGGGGSGGGGSEAAAK |
| for exemplary linker | | |
| amino acid sequence | 948 | GGGGSEAAAKGGGGSEAAAKGGGGS |
| for exemplary linker | | |
| amino acid sequence | 949 | GGGGSEAAAKGGGGSEAAAKEAAAK |
| for exemplary linker | | |
| amino acid sequence | 950 | GGGGSEAAAKEAAAKGGGGSGGGGS |
| for exemplary linker | | |
| amino acid sequence | 951 | GGGGSEAAAKEAAAKEAAAKGGGGS |
| for exemplary linker | | |
| amino acid sequence | 952 | GGGGSEAAAKEAAAKEAAAKEAAAK |
| for exemplary linker | | |
| amino acid sequence | 953 | EAAAKGGGGSGGGGSGGGGSGGGGS |
| for exemplary linker | | |
| amino acid sequence | 954 | EAAAKGGGGSGGGGGGGGSEAAAK |
| for exemplary linker | | |
| amino acid sequence | 955 | EAAAKGGGGSGGGGSEAAAKGGGGS |
| for exemplary linker | | |
| amino acid sequence | 956 | EAAAKGGGGSGGGGSEAAAKEAAAK |
| for exemplary linker | | |
| amino acid sequence | 957 | EAAAKGGGGSEAAAKGGGGSGGGGS |
| for exemplary linker | | |
| amino acid sequence | 958 | EAAAKGGGGSEAAAKGGGGSEAAAK |
| for exemplary linker | | |
| amino acid sequence | 959 | EAAAKGGGGSEAAAKEAAAKGGGGS |
| for exemplary linker | | |
| amino acid sequence | 960 | EAAAKGGGGSEAAAKEAAAKEAAAK |
| for exemplary linker | | |
| amino acid sequence | 961 | EAAAKEAAAKGGGGSEAAAKGGGGS |
| for exemplary linker | | |
| amino acid sequence | 962 | EAAAKEAAAKGGGGSEAAAKEAAAK |
| for exemplary linker | | |
| amino acid sequence | 963 | EAAAKEAAAKEAAAKGGGGSEAAAK |
| for exemplary linker | | |
| amino acid sequence | 964 | EAAAKEAAAKEAAAKEAAAKGGGGS |
| for exemplary linker | | |
| amino acid sequence | 965 | EAAAKEAAAKEAAAKEAAAKEAAAK |
| for exemplary linker | | |
| amino acid sequence | 966 | GTKDSTKDIPETPSKD |
| for exemplary linker | | |
| amino acid sequence | 967 | GRDVRQPEVKEEKPES |
| for exemplary linker | | |
| amino acid sequence | 968 | EGKSSGSGSESKSTAG |
| for exemplary linker | | |
| amino acid sequence | 969 | TPGS PAGS PTSTEEGT |
| for exemplary linker | | |
| amino acid sequence | 970 | GSEPATSGSETPGTST |
| for exemplary linker | | |
| mRNA encoding BC22n | 972 | GGGAAGCUCAGAAUAAACGCUCAACUUUGGCCGGAUCUGCCACCAUGGAGGCCUCCCCCGCCUCCGGCCCCCGGCACCUGAUGGA |
| | | CCCCCACAUCUUCACCUCCAACUUCAACAACGGCAUCGGCCGGCACAAGACCUACCUGUGCUACGAGGUGGAGCGGCUGGACAAC |
| | | GGCACCUCCGUGAAGAUGGACCAGCACCGGGGCUUCCUGCACAACCAGGCCAAGAACCUGCUGUGCGGCUUCUACGGCCGGCACG |
| | | CCGAGCUGCGGUUCCUGGACCUGGUGCCCUCCCUGCAGCUGGACCCCGCCCAGAUCUACCGGGUGACCUGGUUCAUCUCCUGGUC |
| | | CCCCUGCUUCUCCUGGGGCUGCGCCGGCGAGGUGCGGGCCUUCCUGCAGGAGAACACCCACGUGCGGCUGCGGAUCUUCGCCGCC |
| | | CGGAUCUACGACUACGACCCCCUGUACAAGGAGGCCCUGCAGAUGCUGCGGGACGCCGGCGCCCAGGUGUCCAUCAUGACCUACG |
| | | ACGAGUUCAAGCACUGCUGGGACACCUUCGUGGACCACCAGGGCUGCCCCUUCCAGCCCUGGGACGGCCUGGACGAGCACUCCCA |
| | | GGCCCUGUCCGGCCGGCUGCGGGCCAUCCUGCAGAACCAGGGCAACUCCGGCUCCGAGACCCCCGGCACCUCCGAGUCCGCCACC |
| | | CCCGAGUCCGACAAGAAGUACUCCAUCGGCCUGGCCAUCGGCACCAACUCCGUGGGCUGGGCCGUGAUCACCGACGAGUACAAGG |
| | | UGCCCUCCAAGAAGUUCAAGGUGCUGGGCAACACCGACCGGCACUCCAUCAAGAAGAACCUGAUCGGCGCCCUGCUGUUCGACUC |
| | | CGGCGAGACCGCCGAGGCCACCCGGCUGAAGCGGACCGCCCGGCGGCGGUACACCCGGCGGAAGAACCGGAUCUGCUACCUGCAG |
| | | GAGAUCUUCUCCAACGAGAUGGCCAAGGUGGACGACUCCUUCUUCCACCGGCUGGAGGAGUCCUUCCUGGUGGAGGAGGACAAGA |
| | | AGCACGAGCGGCACCCCAUCUUCGGCAACAUCGUGGACGAGGUGGCCUACCACGAGAAGUACCCCACCAUCUACCACCUGCGGAA |
| | | GAAGCUGGUGGACUCCACCGACAAGGCCGACCUGCGGCUGAUCUACCUGGCCCUGGCCCACAUGAUCAAGUUCCGGGGCCACUUC |
| | | CUGAUCGAGGGCGACCUGAACCCCGACAACUCCGACGUGGACAAGCUGUUCAUCCAGCUGGUGCAGACCUACAACCAGCUGUUCG |
| | | AGGAGAACCCCAUCAACGCCUCCGGCGUGGACGCCAAGGCCAUCCUGUCCGCCCGGCUGUCCAAGUCCCGGCGGCUGGAGAACCU |
| | | GAUCGCCCAGCUGCCCGGCGAGAAGAAGAACGGCCUGUUCGGCAACCUGAUCGCCCUGUCCCUGGGCCUGACCCCCAACUUCAAG |
| | | UCCAACUUCGACCUGGCCGAGGACGCCAAGCUGCAGCUGUCCAAGGACACCUACGACGACGACCUGGACAACCUGCUGGCCCAGA |
| | | UCGGCGACCAGUACGCCGACCUGUUCCUGGCCGCCAAGAACCUGUCCGACGCCAUCCUGCUGUCCGACAUCCUGCGGGUGAACAC |
| | | CGAGAUCACCAAGGCCCCCCUGUCCGCCUCCAUGAUCAAGCGGUACGACGAGCACCACCAGGACCUGACCCUGCUGAAGGCCCUG |
| | | GUGCGGCAGCAGCUGCCCGAGAAGUACAAGGAGAUCUUCUUCGACCAGUCCAAGAACGGCUACGCCGGCUACAUCGACGGCGGCG |
| | | CCUCCCAGGAGGAGUUCUACAAGUUCAUCAAGCCCAUCCUGGAGAAGAUGGACGGCACCGAGGAGCUGCUGGUGAAGCUGAACCG |
| | | GGAGGACCUGCUGCGGAAGCAGCGGACCUUCGACAACGGCUCCAUCCCCCACCAGAUCCACCUGGGCGAGCUGCACGCCAUCCUG |
| | | CGGCGGCAGGAGGACUUCUACCCCUUCCUGAAGGACAACCGGGAGAAGAUCGAGAAGAUCCUGACCUUCCGGAUCCCCUACUACG |
| | | UGGGCCCCCUGGCCCGGGGCAACUCCCGGUUCGCCUGGAUGACCCGGAAGUCCGAGGAGACCAUCACCCCCUGGAACUUCGAGGA |
| | | GGUGGUGGACAAGGGCGCCUCCGCCCAGUCCUUCAUCGAGCGGAUGACCAACUUCGACAAGAACCUGCCCAACGAGAAGGUGCUG |
| | | CCCAAGCACUCCCUGCUGUACGAGUACUUCACCGUGUACAACGAGCUGACCAAGGUGAAGUACGUGACCGAGGGCAUGCGGAAGC |
| | | CCGCCUUCCUGUCCGGCGAGCAGAAGAAGGCCAUCGUGGACCUGCUGUUCAAGACCAACCGGAAGGUGACCGUGAAGCAGCUGAA |
| | | GGAGGACUACUUCAAGAAGAUCGAGUGCUUCGACUCCGUGGAGAUCUCCGGCGUGGAGGACCGGUUCAACGCCUCCCUGGGCACC |
| | | UACCACGACCUGCUGAAGAUCAUCAAGGACAAGGACUUCCUGGACAACGAGGAGAACGAGGACAUCCUGGAGGACAUCGUGCUGA |
| | | CCCUGACCCUGUUCGAGGACCGGGAGAUGAUCGAGGAGCGGCUGAAGACCUACGCCCACCUGUUCGACGACAAGGUGAUGAAGCA |
| | | GCUGAAGCGGCGGCGGUACACCGGCUGGGGCCGGCUGUCCCGGAAGCUGAUCAACGGCAUCCGGGACAAGCAGUCCGGCAAGACC |
| | | AUCCUGGACUUCCUGAAGUCCGACGGCUUCGCCAACCGGAACUUCAUGCAGCUGAUCCACGACGACUCCCUGACCUUCAAGGAGG |
| | | ACAUCCAGAAGGCCCAGGUGUCCGGCCAGGGCGACUCCCUGCACGAGCACAUCGCCAACCUGGCCGGCUCCCCCGCCAUCAAGAA |
| | | GGGCAUCCUGCAGACCGUGAAGGUGGUGGACGAGCUGGUGAAGGUGAUGGGCCGGCACAAGCCCGAGAACAUCGUGAUCGAGAUG |
| | | GCCCGGGAGAACCAGACCACCCAGAAGGGCCAGAAGAACUCCCGGGAGCGGAUGAAGCGGAUCGAGGAGGGCAUCAAGGAGCUGG |
| | | GCUCCCAGAUCCUGAAGGAGCACCCCGUGGAGAACACCCAGCUGCAGAACGAGAAGCUGUACCUGUACUACCUGCAGAACGGCCG |
| | | GGACAUGUACGUGGACCAGGAGCUGGACAUCAACCGGCUGUCCGACUACGACGUGGACCACAUCGUGCCCCAGUCCUUCCUGAAG |
| | | GACGACUCCAUCGACAACAAGGUGCUGACCCGGUCCGACAAGAACCGGGGCAAGUCCGACAACGUGCCCUCCGAGGAGGUGGUGA |
| | | AGAAGAUGAAGAACUACUGGCGGCAGCUGCUGAACGCCAAGCUGAUCACCCAGCGGAAGUUCGACAACCUGACCAAGGCCGAGCG |
| | | GGGCGGCCUGUCCGAGCUGGACAAGGCCGGCUUCAUCAAGCGGCAGCUGGUGGAGACCCGGCAGAUCACCAAGCACGUGGCCCAG |
| | | AUCCUGGACUCCCGGAUGAACACCAAGUACGACGAGAACGACAAGCUGAUCCGGGAGGUGAAGGUGAUCACCCUGAAGUCCAAGC |
| | | UGGUGUCCGACUUCCGGAAGGACUUCCAGUUCUACAAGGUGCGGGAGAUCAACAACUACCACCACGCCCACGACGCCUACCUGAA |
| | | CGCCGUGGUGGGCACCGCCCUGAUCAAGAAGUACCCCAAGCUGGAGUCCGAGUUCGUGUACGGCGACUACAAGGUGUACGACGUG |
| | | CGGAAGAUGAUCGCCAAGUCCGAGCAGGAGAUCGGCAAGGCCACCGCCAAGUACUUCUUCUACUCCAACAUCAUGAACUUCUUCA |
| | | AGACCGAGAUCACCCUGGCCAACGGCGAGAUCCGGAAGCGGCCCCUGAUCGAGACCAACGGCGAGACCGGCGAGAUCGUGUGGGA |
| | | CAAGGGCCGGGACUUCGCCACCGUGCGGAAGGUGCUGUCCAUGCCCCAGGUGAACAUCGUGAAGAAGACCGAGGUGCAGACCGGC |
| | | GGCUUCUCCAAGGAGUCCAUCCUGCCCAAGCGGAACUCCGACAAGCUGAUCGCCCGGAAGAAGGACUGGGACCCCAAGAAGUACG |
| | | GCGGCUUCGACUCCCCCACCGUGGCCUACUCCGUGCUGGUGGUGGCCAAGGUGGAGAAGGGCAAGUCCAAGAAGCUGAAGUCCGU |
| | | GAAGGAGCUGCUGGGCAUCACCAUCAUGGAGCGGUCCUCCUUCGAGAAGAACCCCAUCGACUUCCUGGAGGCCAAGGGCUACAAG |
| | | GAGGUGAAGAAGGACCUGAUCAUCAAGCUGCCCAAGUACUCCCUGUUCGAGCUGGAGAACGGCCGGAAGCGGAUGCUGGCCUCCG |
| | | CCGGCGAGCUGCAGAAGGGCAACGAGCUGGCCCUGCCCUCCAAGUACGUGAACUUCCUGUACCUGGCCUCCCACUACGAGAAGCU |
| | | GAAGGGCUCCCCCGAGGACAACGAGCAGAAGCAGCUGUUCGUGGAGCAGCACAAGCACUACCUGGACGAGAUCAUCGAGCAGAUC |
| | | UCCGAGUUCUCCAAGCGGGUGAUCCUGGCCGACGCCAACCUGGACAAGGUGCUGUCCGCCUACAACAAGCACCGGGACAAGCCCA |
| | | UCCGGGAGCAGGCCGAGAACAUCAUCCACCUGUUCACCCUGACCAACCUGGGCGCCCCCGCCGCCUUCAAGUACUUCGACACCAC |
| | | CAUCGACCGGAAGCGGUACACCUCCACCAAGGAGGUGCUGGACGCCACCCUGAUCCACCAGUCCAUCACCGGCCUGUACGAGACC |
| | | CGGAUCGACCUGUCCCAGCUGGGCGGCGACGGCGGCGGCUCCCCCAAGAAGAAGCGGAAGGUGUGACUAGCACCAGCCUCAAGAA |
| | | CACCCGAAUGGAGUCUCUAAGCUACAUAAUACCAACUUACACUUUACAAAAUGUUGUCCCCCAAAAUGUAGCCAUUCGUAUCUGC |
| | | UCCUAAUAAAAAGAAAGUUUCUUCACAUUCUCUCGAGAAAAAAAAAAAAUGGAAAAAAAAAAAACGGAAAAAAAAAAAAGGUAAA |
| | | AAAAAAAAAUAUAAAAAAAAAAAACAUAAAAAAAAAAAACGAAAAAAAAAAAACGUAAAAAAAAAAAACUCAAAAAAAAAAAAGA |
| | | UAAAAAAAAAAAACCUAAAAAAAAAAAAUGUAAAAAAAAAAAAGGGAAAAAAAAAAAACGCAAAAAAAAAAAACACAAAAAAAAA |
| | | AAAUGCAAAAAAAAAAAAUCGAAAAAAAAAAAAUCUAAAAAAAAAAAACGAAAAAAAAAAAACCCAAAAAAAAAAAAGACAAAAA |
| | | AAAAAAAUAGAAAAAAAAAAAAGUUAAAAAAAAAAAACUGAAAAAAAAAAAAUUUAAAAAAAAAAAAUCUAG |
| mRNA encoding BC22n | 973 | GGGAAGCUCAGAAUAAACGCUCAACUUUGGCCGGAUCUGCCACCAUGGAGGCCUCCCCCGCCUCCGGCCCCCGGCACCUGAUGGA |
| with HiBit tag | | CCCCCACAUCUUCACCUCCAACUUCAACAACGGCAUCGGCCGGCACAAGACCUACCUGUGCUACGAGGUGGAGCGGCUGGACAAC |
| | | GGCACCUCCGUGAAGAUGGACCAGCACCGGGGCUUCCUGCACAACCAGGCCAAGAACCUGCUGUGCGGCUUCUACGGCCGGCACG |
| | | CCGAGCUGCGGUUCCUGGACCUGGUGCCCUCCCUGCAGCUGGACCCCGCCCAGAUCUACCGGGUGACCUGGUUCAUCUCCUGGUC |
| | | CCCCUGCUUCUCCUGGGGCUGCGCCGGCGAGGUGCGGGCCUUCCUGCAGGAGAACACCCACGUGCGGCUGCGGAUCUUCGCCGCC |
| | | CGGAUCUACGACUACGACCCCCUGUACAAGGAGGCCCUGCAGAUGCUGCGGGACGCCGGCGCCCAGGUGUCCAUCAUGACCUACG |
| | | ACGAGUUCAAGCACUGCUGGGACACCUUCGUGGACCACCAGGGCUGCCCCUUCCAGCCCUGGGACGGCCUGGACGAGCACUCCCA |
| | | GGCCCUGUCCGGCCGGCUGCGGGCCAUCCUGCAGAACCAGGGCAACUCCGGCUCCGAGACCCCCGGCACCUCCGAGUCCGCCACC |
| | | CCCGAGUCCGACAAGAAGUACUCCAUCGGCCUGGCCAUCGGCACCAACUCCGUGGGCUGGGCCGUGAUCACCGACGAGUACAAGG |
| | | UGCCCUCCAAGAAGUUCAAGGUGCUGGGCAACACCGACCGGCACUCCAUCAAGAAGAACCUGAUCGGCGCCCUGCUGUUCGACUC |
| | | CGGCGAGACCGCCGAGGCCACCCGGCUGAAGCGGACCGCCCGGCGGCGGUACACCCGGCGGAAGAACCGGAUCUGCUACCUGCAG |
| | | GAGAUCUUCUCCAACGAGAUGGCCAAGGUGGACGACUCCUUCUUCCACCGGCUGGAGGAGUCCUUCCUGGUGGAGGAGGACAAGA |
| | | AGCACGAGCGGCACCCCAUCUUCGGCAACAUCGUGGACGAGGUGGCCUACCACGAGAAGUACCCCACCAUCUACCACCUGCGGAA |
| | | GAAGCUGGUGGACUCCACCGACAAGGCCGACCUGCGGCUGAUCUACCUGGCCCUGGCCCACAUGAUCAAGUUCCGGGGCCACUUC |
| | | CUGAUCGAGGGCGACCUGAACCCCGACAACUCCGACGUGGACAAGCUGUUCAUCCAGCUGGUGCAGACCUACAACCAGCUGUUCG |
| | | AGGAGAACCCCAUCAACGCCUCCGGCGUGGACGCCAAGGCCAUCCUGUCCGCCCGGCUGUCCAAGUCCCGGCGGCUGGAGAACCU |
| | | GAUCGCCCAGCUGCCCGGCGAGAAGAAGAACGGCCUGUUCGGCAACCUGAUCGCCCUGUCCCUGGGCCUGACCCCCAACUUCAAG |
| | | UCCAACUUCGACCUGGCCGAGGACGCCAAGCUGCAGCUGUCCAAGGACACCUACGACGACGACCUGGACAACCUGCUGGCCCAGA |
| | | UCGGCGACCAGUACGCCGACCUGUUCCUGGCCGCCAAGAACCUGUCCGACGCCAUCCUGCUGUCCGACAUCCUGCGGGUGAACAC |
| | | CGAGAUCACCAAGGCCCCCCUGUCCGCCUCCAUGAUCAAGCGGUACGACGAGCACCACCAGGACCUGACCCUGCUGAAGGCCCUG |
| | | GUGCGGCAGCAGCUGCCCGAGAAGUACAAGGAGAUCUUCUUCGACCAGUCCAAGAACGGCUACGCCGGCUACAUCGACGGCGGCG |
| | | CCUCCCAGGAGGAGUUCUACAAGUUCAUCAAGCCCAUCCUGGAGAAGAUGGACGGCACCGAGGAGCUGCUGGUGAAGCUGAACCG |
| | | GGAGGACCUGCUGCGGAAGCAGCGGACCUUCGACAACGGCUCCAUCCCCCACCAGAUCCACCUGGGCGAGCUGCACGCCAUCCUG |
| | | CGGCGGCAGGAGGACUUCUACCCCUUCCUGAAGGACAACCGGGAGAAGAUCGAGAAGAUCCUGACCUUCCGGAUCCCCUACUACG |
| | | UGGGCCCCCUGGCCCGGGGCAACUCCCGGUUCGCCUGGAUGACCCGGAAGUCCGAGGAGACCAUCACCCCCUGGAACUUCGAGGA |
| | | GGUGGUGGACAAGGGCGCCUCCGCCCAGUCCUUCAUCGAGCGGAUGACCAACUUCGACAAGAACCUGCCCAACGAGAAGGUGCUG |
| | | CCCAAGCACUCCCUGCUGUACGAGUACUUCACCGUGUACAACGAGCUGACCAAGGUGAAGUACGUGACCGAGGGCAUGCGGAAGC |
| | | CCGCCUUCCUGUCCGGCGAGCAGAAGAAGGCCAUCGUGGACCUGCUGUUCAAGACCAACCGGAAGGUGACCGUGAAGCAGCUGAA |
| | | GGAGGACUACUUCAAGAAGAUCGAGUGCUUCGACUCCGUGGAGAUCUCCGGCGUGGAGGACCGGUUCAACGCCUCCCUGGGCACC |
| | | UACCACGACCUGCUGAAGAUCAUCAAGGACAAGGACUUCCUGGACAACGAGGAGAACGAGGACAUCCUGGAGGACAUCGUGCUGA |
| | | CCCUGACCCUGUUCGAGGACCGGGAGAUGAUCGAGGAGCGGCUGAAGACCUACGCCCACCUGUUCGACGACAAGGUGAUGAAGCA |
| | | GCUGAAGCGGCGGCGGUACACCGGCUGGGGCCGGCUGUCCCGGAAGCUGAUCAACGGCAUCCGGGACAAGCAGUCCGGCAAGACC |
| | | AUCCUGGACUUCCUGAAGUCCGACGGCUUCGCCAACCGGAACUUCAUGCAGCUGAUCCACGACGACUCCCUGACCUUCAAGGAGG |
| | | ACAUCCAGAAGGCCCAGGUGUCCGGCCAGGGCGACUCCCUGCACGAGCACAUCGCCAACCUGGCCGGCUCCCCCGCCAUCAAGAA |
| | | GGGCAUCCUGCAGACCGUGAAGGUGGUGGACGAGCUGGUGAAGGUGAUGGGCCGGCACAAGCCCGAGAACAUCGUGAUCGAGAUG |
| | | GCCCGGGAGAACCAGACCACCCAGAAGGGCCAGAAGAACUCCCGGGAGCGGAUGAAGCGGAUCGAGGAGGGCAUCAAGGAGCUGG |
| | | GCUCCCAGAUCCUGAAGGAGCACCCCGUGGAGAACACCCAGCUGCAGAACGAGAAGCUGUACCUGUACUACCUGCAGAACGGCCG |
| | | GGACAUGUACGUGGACCAGGAGCUGGACAUCAACCGGCUGUCCGACUACGACGUGGACCACAUCGUGCCCCAGUCCUUCCUGAAG |
| | | GACGACUCCAUCGACAACAAGGUGCUGACCCGGUCCGACAAGAACCGGGGCAAGUCCGACAACGUGCCCUCCGAGGAGGUGGUGA |
| | | AGAAGAUGAAGAACUACUGGCGGCAGCUGCUGAACGCCAAGCUGAUCACCCAGCGGAAGUUCGACAACCUGACCAAGGCCGAGCG |
| | | GGGCGGCCUGUCCGAGCUGGACAAGGCCGGCUUCAUCAAGCGGCAGCUGGUGGAGACCCGGCAGAUCACCAAGCACGUGGCCCAG |
| | | AUCCUGGACUCCCGGAUGAACACCAAGUACGACGAGAACGACAAGCUGAUCCGGGAGGUGAAGGUGAUCACCCUGAAGUCCAAGC |
| | | UGGUGUCCGACUUCCGGAAGGACUUCCAGUUCUACAAGGUGCGGGAGAUCAACAACUACCACCACGCCCACGACGCCUACCUGAA |
| | | CGCCGUGGUGGGCACCGCCCUGAUCAAGAAGUACCCCAAGCUGGAGUCCGAGUUCGUGUACGGCGACUACAAGGUGUACGACGUG |
| | | CGGAAGAUGAUCGCCAAGUCCGAGCAGGAGAUCGGCAAGGCCACCGCCAAGUACUUCUUCUACUCCAACAUCAUGAACUUCUUCA |
| | | AGACCGAGAUCACCCUGGCCAACGGCGAGAUCCGGAAGCGGCCCCUGAUCGAGACCAACGGCGAGACCGGCGAGAUCGUGUGGGA |
| | | CAAGGGCCGGGACUUCGCCACCGUGCGGAAGGUGCUGUCCAUGCCCCAGGUGAACAUCGUGAAGAAGACCGAGGUGCAGACCGGC |
| | | GGCUUCUCCAAGGAGUCCAUCCUGCCCAAGCGGAACUCCGACAAGCUGAUCGCCCGGAAGAAGGACUGGGACCCCAAGAAGUACG |
| | | GCGGCUUCGACUCCCCCACCGUGGCCUACUCCGUGCUGGUGGUGGCCAAGGUGGAGAAGGGCAAGUCCAAGAAGCUGAAGUCCGU |
| | | GAAGGAGCUGCUGGGCAUCACCAUCAUGGAGCGGUCCUCCUUCGAGAAGAACCCCAUCGACUUCCUGGAGGCCAAGGGCUACAAG |
| | | GAGGUGAAGAAGGACCUGAUCAUCAAGCUGCCCAAGUACUCCCUGUUCGAGCUGGAGAACGGCCGGAAGCGGAUGCUGGCCUCCG |
| | | CCGGCGAGCUGCAGAAGGGCAACGAGCUGGCCCUGCCCUCCAAGUACGUGAACUUCCUGUACCUGGCCUCCCACUACGAGAAGCU |
| | | GAAGGGCUCCCCCGAGGACAACGAGCAGAAGCAGCUGUUCGUGGAGCAGCACAAGCACUACCUGGACGAGAUCAUCGAGCAGAUC |
| | | UCCGAGUUCUCCAAGCGGGUGAUCCUGGCCGACGCCAACCUGGACAAGGUGCUGUCCGCCUACAACAAGCACCGGGACAAGCCCA |
| | | UCCGGGAGCAGGCCGAGAACAUCAUCCACCUGUUCACCCUGACCAACCUGGGCGCCCCCGCCGCCUUCAAGUACUUCGACACCAC |
| | | CAUCGACCGGAAGCGGUACACCUCCACCAAGGAGGUGCUGGACGCCACCCUGAUCCACCAGUCCAUCACCGGCCUGUACGAGACC |
| | | CGGAUCGACCUGUCCCAGCUGGGCGGCGACGGCGGCGGCUCCCCCAAGAAGAAGCGGAAGGUGUCCGAGUCCGCCACCCCCGAGU |
| | | CCGUGUCCGGCUGGCGGCUGUUCAAGAAGAUCUCCUGACUAGCACCAGCCUCAAGAACACCCGAAUGGAGUCUCUAAGCUACAUA |
| | | AUACCAACUUACACUUUACAAAAUGUUGUCCCCCAAAAUGUAGCCAUUCGUAUCUGCUCCUAAUAAAAAGAAAGUUUCUUCACAU |
| | | UCUCUCGAGAAAAAAAAAAAAUGGAAAAAAAAAAAACGGAAAAAAAAAAAAGGUAAAAAAAAAAAAUAUAAAAAAAAAAAACAUA |
| | | AAAAAAAAAAACGAAAAAAAAAAAACGUAAAAAAAAAAAACUCAAAAAAAAAAAAGAUAAAAAAAAAAAACCUAAAAAAAAAAAA |
| | | UGUAAAAAAAAAAAAGGGAAAAAAAAAAAACGCAAAAAAAAAAAACACAAAAAAAAAAAAUGCAAAAAAAAAAAAUCGAAAAAAA |
| | | AAAAAUCUAAAAAAAAAAAACGAAAAAAAAAAAACCCAAAAAAAAAAAAGACAAAAAAAAAAAAUAGAAAAAAAAAAAAGUUAAA |
| | | AAAAAAAAACUGAAAAAAAAAAAAUUUAAAAAAAAAAAAUCUAG |
| | 974 | Not used |
| mRNA encoding UGI | 975 | GGGAGACCCAAGCUGGCUAGCUCCCGCAGUCGGCGUCCAGCGGCUCUGCUUGUUCGUGUGUGUGUCGUUGCAGGCCUUAUUCGGA |
| | | UCCGCCACCAUGGGACCGAAGAAGAAGAGAAAGGUCGGAGGAGGAAGCACAAACCUGUCGGACAUCAUCGAAAAGGAAACAGGAA |
| | | AGCAGCUGGUCAUCCAGGAAUCGAUCCUGAUGCUGCCGGAAGAAGUCGAAGAAGUCAUCGGAAACAAGCCGGAAUCGGACAUCCU |
| | | GGUCCACACAGCAUACGACGAAUCGACAGACGAAAACGUCAUGCUGCUGACAUCGGACGCACCGGAAUACAAGCCGUGGGCACUG |
| | | GUCAUCCAGGACUCGAACGGAGAAAACAAGAUCAAGAUGCUGUGAUAGUCUAGACAUCACAUUUAAAAGCAUCUCAGCCUACCAU |
| | | GAGAAUAAGAGAAAGAAAAUGAAGAUCAAUAGCUUAUUCAUCUCUUUUUCUUUUUCGUUGGUGUAAAGCCAACACCCUGUCUAAA |
| | | AAACAUAAAUUUCUUUAAUCAUUUUGCCUCUUUUCUCUGUGCUUCAAUUAAUAAAAAAUGGAAAGAACCUCGAGUCUAG |
| Exemplary amino acid | 976 | MDGSGGGSPKKKRKVEDKRPAATKKAGQAKKKKGGSGGGEASPASGPRHLMDPHIFTSNFNNGIGRHKTYLCYEVERLDNGTSVK |
| sequence for NLS-NLS- | | MDQHRGFLHNQAKNLLCGFYGRHAELRFLDLVPSLQLDPAQIYRVTWFISWSPCFSWGCAGEVRAFLQENTHVRLRIFAARIYDY |
| APOBEC3A-L070- | | DPLYKEALQMLRDAGAQVSIMTYDEFKHCWDTFVDHQGCPFQPWDGLDEHSQALSGRLRAILQNQGNGTKDSTKDIPETPSKDAA |
| Nme2D16A | | FKPNPINYILGLAIGIASVGWAMVEIDEEENPIRLIDLGVRVFERAEVPKTGDSLAMARRLARSVRRLTRRRAHRLLRARRLLKR |
| | | EGVLQAADFDENGLIKSLPNTPWQLRAAALDRKLTPLEWSAVLLHLIKHRGYLSQRKNEGETADKELGALLKGVANNAHALQTGD |
| | | FRTPAELALNKFEKESGHIRNQRGDYSHTFSRKDLQAELILLFEKQKEFGNPHVSGGLKEGIETLLMTQRPALSGDAVQKMLGHC |
| | | TFEPAEPKAAKNTYTAERFIWLTKLNNLRILEQGSERPLTDTERATLMDEPYRKSKLTYAQARKLLGLEDTAFFKGLRYGKDNAE |
| | | ASTLMEMKAYHAISRALEKEGLKDKKSPLNLSSELQDEIGTAFSLEKTDEDITGRLKDRVQPEILEALLKHISFDKFVQISLKAL |
| | | RRIVPLMEQGKRYDEACAEIYGDHYGKKNTEEKIYLPPIPADEIRNPVVLRALSQARKVINGVVRRYGSPARIHIETAREVGKSF |
| | | KDRKEIEKRQEENRKDREKAAAKFREYFPNFVGEPKSKDILKLRLYEQQHGKCLYSGKEINLVRLNEKGYVEIDHALPFSRTWDD |
| | | SFNNKVLVLGSENQNKGNQTPYEYFNGKDNSREWQEFKARVETSRFPRSKKQRILLQKFDEDGFKECNLNDTRYVNRFLCQFVAD |
| | | HILLTGKGKRRVFASNGQITNLLRGFWGLRKVRAENDRHHALDAVVVACSTVAMQQKITRFVRYKEMNAFDGKTIDKETGKVLHQ |
| | | KTHFPQPWEFFAQEVMIRVFGKPDGKPEFEEADTPEKLRTLLAEKLSSRPEAVHEYVTPLFVSRAPNRKMSGAHKDTLRSAKRFV |
| | | KHNEKISVKRVWLTEIKLADLENMVNYKNGREIELYEALKARLEAYGGNAKQAFDPKDNPFYKKGGQLVKAVRVEKTQESGVLLN |
| | | KKNAYTIADNGDMVRVDVFCKVDKKGKNQYFIVPIYAWQVAENILPDIDCKGYRIDDSYTFCFSLHKYDLIAFQKDEKSKVEFAY |
| | | YINCDSSNGRFYLAWHDKGSKEQQFRISTQNLVLIQKYQVNELGKEIRPCRLKKRPPVR |
| Amino acid sequence | 977 | MSSETGPVAVDPTLRRRIEPHEFEVFEDPRELRKETCLLYEINWGGRHSIWRHTSQNTNKHVEVNFIEKFTTERYFCPNTRCSIT |
| for BE3 | | WFLSWSPCGECSRAITEFLSRYPHVTLFIYIARLYHHADPRNRQGLRDLISSGVTIQIMTEQESGYCWRNFVNYSPSNEAHWPRY |
| | | PHLWVRLYVLELYCIILGLPPCLNILRRKQPQLTFFTIALQSCHYQRLPPHILWATGLKSGSETPGTSESATPESDKKYSIGLAI |
| | | GTNSVGWAVITDEYKVPSKKFKVLGNTDRHSIKKNLIGALLFDSGETAEATRLKRTARRRYTRRKNRICYLQEIFSNEMAKVDDS |
| | | FFHRLEESELVEEDKKHERHPIFGNIVDEVAYHEKYPTIYHLRKKLVDSTDKADLRLIYLALAHMIKFRGHFLIEGDLNPDNSDV |
| | | DKLFIQLVQTYNQLFEENPINASGVDAKAILSARLSKSRRLENLIAQLPGEKKNGLFGNLIALSLGLTPNEKSNFDLAEDAKLQL |
| | | SKDTYDDDLDNLLAQIGDQYADLFLAAKNLSDAILLSDILRVNTEITKAPLSASMIKRYDEHHQDLTLLKALVRQQLPEKYKEIF |
| | | FDQSKNGYAGYIDGGASQEEFYKFIKPILEKMDGTEELLVKLNREDLLRKQRTFDNGSIPHQIHLGELHAILRRQEDFYPFLKDN |
| | | REKIEKILTFRIPYYVGPLARGNSRFAWMTRKSEETITPWNFEEVVDKGASAQSFIERMTNEDKNLPNEKVLPKHSLLYEYFTVY |
| | | NELTKVKYVTEGMRKPAFLSGEQKKAIVDLLEKTNRKVTVKQLKEDYFKKIECFDSVEISGVEDRFNASLGTYHDLLKIIKDKDF |
| | | LDNEENEDILEDIVLTLTLFEDREMIEERLKTYAHLFDDKVMKQLKRRRYTGWGRLSRKLINGIRDKQSGKTILDFLKSDGFANR |
| | | NFMQLIHDDSLTFKEDIQKAQVSGQGDSLHEHIANLAGSPAIKKGILQTVKVVDELVKVMGRHKPENIVIEMARENQTTQKGQKN |
| | | SRERMKRIEEGIKELGSQILKEHPVENTQLQNEKLYLYYLQNGRDMYVDQELDINRLSDYDVDHIVPQSFLKDDSIDNKVLTRSD |
| | | KNRGKSDNVPSEEVVKKMKNYWRQLLNAKLITQRKFDNLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVGTALIKKYPKLESEFVYGDYKVYDVRKMIAKSEQEIGK |
| | | ATAKYFFYSNIMNFFKTEITLANGEIRKRPLIETNGETGEIVWDKGRDFATVRKVLSMPQVNIVKKTEVQTGGFSKESILPKRNS |
| | | DKLIARKKDWDPKKYGGFDSPTVAYSVLVVAKVEKGKSKKLKSVKELLGITIMERSSFEKNPIDFLEAKGYKEVKKDLIIKLPKY |
| | | SLFELENGRKRMLASAGELQKGNELALPSKYVNFLYLASHYEKLKGSPEDNEQKQLFVEQHKHYLDEIIEQISEFSKRVILADAN |
| | | LDKVLSAYNKHRDKPIREQAENIIHLFTLTNLGAPAAFKYFDTTIDRKRYTSTKEVLDATLIHQSITGLYETRIDLSQLGGDSGG |
| | | STNLSDIIEKETGKQLVIQESILMLPEEVEEVIGNKPESDILVHTAYDESTDENVMLLTSDAPEYKPWALVIQDSNGENKIKMLS |
| | | GGSPKKKRKV |
| Amino acid sequence | 978 | MSSETGPVAVDPTLRRRIEPHEFEVFFDPRELRKETCLLYEINWGGRHSIWRHTSQNTNKHVEVNFIEKFTTERYFCPNTRCSIT |
| for BE3 | | WFLSWSPCGECSRAITEFLSRYPHVTLFIYIARLYHHADPRNRQGLRDLISSGVTIQIMTEQESGYCWRNFVNYSPSNEAHWPRY |
| | | PHLWVRLYVLELYCIILGLPPCLNILRRKQPQLTFFTIALQSCHYQRLPPHILWATGLKSGSETPGTSESATPESDKKYSIGLAI |
| | | GTNSVGWAVITDEYKVPSKKFKVLGNTDRHSIKKNLIGALLFDSGETAEATRLKRTARRRYTRRKNRICYLQEIFSNEMAKVDDS |
| | | FFHRLEESFLVEEDKKHERHPIFGNIVDEVAYHEKYPTIYHLRKKLVDSTDKADLRLIYLALAHMIKERGHFLIEGDLNPDNSDV |
| | | DKLFIQLVQTYNQLFEENPINASGVDAKAILSARLSKSRRLENLIAQLPGEKKNGLFGNLIALSLGLTPNEKSNEDLAEDAKLQL |
| | | SKDTYDDDLDNLLAQIGDQYADLELAAKNLSDAILLSDILRVNTEITKAPLSASMIKRYDEHHQDLTLLKALVRQQLPEKYKEIF |
| | | FDQSKNGYAGYIDGGASQEEFYKFIKPILEKMDGTEELLVKLNREDLLRKQRTFDNGSIPHQIHLGELHAILRRQEDFYPFLKDN |
| | | REKIEKILTFRIPYYVGPLARGNSRFAWMTRKSEETITPWNFEEVVDKGASAQSFIERMTNFDKNLPNEKVLPKHSLLYEYETVY |
| | | NELTKVKYVTEGMRKPAFLSGEQKKAIVDLLFKTNRKVTVKQLKEDYFKKIECFDSVEISGVEDRENASLGTYHDLLKIIKDKDE |
| | | LDNEENEDILEDIVLTLTLFEDREMIEERLKTYAHLEDDKVMKQLKRRRYTGWGRLSRKLINGIRDKQSGKTILDFLKSDGFANR |
| | | NFMQLIHDDSLTFKEDIQKAQVSGQGDSLHEHIANLAGSPAIKKGILQTVKVVDELVKVMGRHKPENIVIEMARENQTTQKGQKN |
| | | SRERMKRIEEGIKELGSQILKEHPVENTQLQNEKLYLYYLQNGRDMYVDQELDINRLSDYDVDHIVPQSFLKDDSIDNKVLTRSD |
| | | KNRGKSDNVPSEEVVKKMKNYWRQLLNAKLITQRKFDNLTKAERGGLSELDKAGFIKRQLVETRQITKHVAQILDSRMNTKYDEN |
| | | DKLIREVKVITLKSKLVSDERKDFQFYKVREINNYHHAHDAYLNAVVGTALIKKYPKLESEFVYGDYKVYDVRKMIAKSEQEIGK |
| | | ATAKYFFYSNIMNFFKTEITLANGEIRKRPLIETNGETGEIVWDKGRDFATVRKVLSMPQVNIVKKTEVQTGGESKESILPKRNS |
| | | DKLIARKKDWDPKKYGGFDSPTVAYSVLVVAKVEKGKSKKLKSVKELLGITIMERSSFEKNPIDFLEAKGYKEVKKDLIIKLPKY |
| | | SLFELENGRKRMLASAGELQKGNELALPSKYVNFLYLASHYEKLKGSPEDNEQKQLFVEQHKHYLDEIIEQISEFSKRVILADAN |
| | | LDKVLSAYNKHRDKPIREQAENIIHLFTLTNLGAPAAFKYEDTTIDRKRYTSTKEVLDATLIHQSITGLYETRIDLSQLGGDSGG |
| | | STNLSDIIEKETGKQLVIQESILMLPEEVEEVIGNKPESDILVHTAYDESTDENVMLLTSDAPEYKPWALVIQDSNGENKIKMLS |
| | | GGSPKKKRKV |
| Amino acid sequence | 979 | MEASPASGPRHLMDPHIFTSNENNGIGRHKTYLCYEVERLDNGTSVKMDQHRGELHNQAKNLLCGFYGRHAELRELDLVPSLQLD |
| for BC22 with 2x UGI | | PAQIYRVTWFISWSPCFSWGCAGEVRAFLQENTHVRLRIFAARIYDYDPLYKEALQMLRDAGAQVSIMTYDEFKHCWDTFVDHQG |
| | | CPFQPWDGLDEHSQALSGRLRAILQNQGNSGSETPGTSESATPESDKKYSIGLAIGTNSVGWAVITDEYKVPSKKFKVLGNTDRH |
| | | SIKKNLIGALLFDSGETAEATRLKRTARRRYTRRKNRICYLQEIFSNEMAKVDDSFFHRLEESELVEEDKKHERHPIFGNIVDEV |
| | | AYHEKYPTIYHLRKKLVDSTDKADLRLIYLALAHMIKFRGHFLIEGDLNPDNSDVDKLFIQLVQTYNQLFEENPINASGVDAKAI |
| | | LSARLSKSRRLENLIAQLPGEKKNGLFGNLIALSLGLTPNFKSNEDLAEDAKLQLSKDTYDDDLDNLLAQIGDQYADLFLAAKNL |
| | | SDAILLSDILRVNTEITKAPLSASMIKRYDEHHQDLTLLKALVRQQLPEKYKEIFFDQSKNGYAGYIDGGASQEEFYKFIKPILE |
| | | KMDGTEELLVKLNREDLLRKQRTFDNGSIPHQIHLGELHAILRRQEDFYPFLKDNREKIEKILTFRIPYYVGPLARGNSRFAWMT |
| | | RKSEETITPWNFEEVVDKGASAQSFIERMTNEDKNLPNEKVLPKHSLLYEYFTVYNELTKVKYVTEGMRKPAFLSGEQKKAIVDL |
| | | LFKTNRKVTVKQLKEDYFKKIECFDSVEISGVEDRENASLGTYHDLLKIIKDKDELDNEENEDILEDIVLTLTLFEDREMIEERL |
| | | KTYAHLFDDKVMKQLKRRRYTGWGRLSRKLINGIRDKQSGKTILDELKSDGFANRNFMQLIHDDSLTFKEDIQKAQVSGQGDSLH |
| | | EHIANLAGSPAIKKGILQTVKVVDELVKVMGRHKPENIVIEMARENQTTQKGQKNSRERMKRIEEGIKELGSQILKEHPVENTQL |
| | | QNEKLYLYYLQNGRDMYVDQELDINRLSDYDVDHIVPQSFLKDDSIDNKVLTRSDKNRGKSDNVPSEEVVKKMKNYWRQLLNAKL |
| | | ITQRKFDNLTKAERGGLSELDKAGFIKRQLVETRQITKHVAQILDSRMNTKYDENDKLIREVKVITLKSKLVSDERKDFQFYKVR |
| | | EINNYHHAHDAYLNAVVGTALIKKYPKLESEFVYGDYKVYDVRKMIAKSEQEIGKATAKYFFYSNIMNFFKTEITLANGEIRKRP |
| | | LIETNGETGEIVWDKGRDFATVRKVLSMPQVNIVKKTEVQTGGFSKESILPKRNSDKLIARKKDWDPKKYGGEDSPTVAYSVLVV |
| | | AKVEKGKSKKLKSVKELLGITIMERSSFEKNPIDFLEAKGYKEVKKDLIIKLPKYSLFELENGRKRMLASAGELQKGNELALPSK |
| | | YVNFLYLASHYEKLKGSPEDNEQKQLFVEQHKHYLDEIIEQISEFSKRVILADANLDKVLSAYNKHRDKPIREQAENIIHLETLT |
| | | NLGAPAAFKYFDTTIDRKRYTSTKEVLDATLIHQSITGLYETRIDLSQLGGDSGGSGGSGGSTNLSDIIEKETGKQLVIQESILM |
| | | LPEEVEEVIGNKPESDILVHTAYDESTDENVMLLTSDAPEYKPWALVIQDSNGENKIKMLSGGSGGSGGSTNLSDIIEKETGKQL |
| | | VIQESILMLPEEVEEVIGNKPESDILVHTAYDESTDENVMLLTSDAPEYKPWALVIQDSNGENKIKMLSGGSKRTADGSEFEPKK |
| | | KRKV |
| Amino acid sequence | 980 | MKRTADGSEFESPKKKRKVSSETGPVAVDPTLRRRIEPHEFEVFFDPRELRKETCLLYEINWGGRHSIWRHTSQNTNKHVEVNFI |
| for BE4MAX protein | | EKFTTERYFCPNTRCSITWELSWSPCGECSRAITEFLSRYPHVTLFIYIARLYHHADPRNRQGLRDLISSGVTIQIMTEQESGYC |
| | | WRNFVNYSPSNEAHWPRYPHLWVRLYVLELYCIILGLPPCLNILRRKQPQLTFFTIALQSCHYQRLPPHILWATGLKSGGSSGGS |
| | | SGSETPGTSESATPESSGGSSGGSDKKYSIGLAIGTNSVGWAVITDEYKVPSKKFKVLGNTDRHSIKKNLIGALLEDSGETAEAT |
| | | RLKRTARRRYTRRKNRICYLQEIFSNEMAKVDDSFFHRLEESFLVEEDKKHERHPIFGNIVDEVAYHEKYPTIYHLRKKLVDSTD |
| | | KADLRLIYLALAHMIKFRGHFLIEGDLNPDNSDVDKLFIQLVQTYNQLFEENPINASGVDAKAILSARLSKSRRLENLIAQLPGE |
| | | KKNGLFGNLIALSLGLTPNEKSNEDLAEDAKLQLSKDTYDDDLDNLLAQIGDQYADLFLAAKNLSDAILLSDILRVNTEITKAPL |
| | | SASMIKRYDEHHQDLTLLKALVRQQLPEKYKEIFFDQSKNGYAGYIDGGASQEEFYKFIKPILEKMDGTEELLVKLNREDLLRKQ |
| | | RTFDNGSIPHQIHLGELHAILRRQEDFYPELKDNREKIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMRKPAFLSGEQKKAIVDLLFKTNRKVTVKQLKEDYFKKI |
| | | ECFDSVEISGVEDRENASLGTYHDLLKIIKDKDFLDNEENEDILEDIVLTLTLFEDREMIEERLKTYAHLEDDKVMKQLKRRRYT |
| | | GWGRLSRKLINGIRDKQSGKTILDELKSDGFANRNFMQLIHDDSLTFKEDIQKAQVSGQGDSLHEHIANLAGSPAIKKGILQTVK |
| | | VVDELVKVMGRHKPENIVIEMARENQTTQKGQKNSRERMKRIEEGIKELGSQILKEHPVENTQLQNEKLYLYYLQNGRDMYVDQE |
| | | LDINRLSDYDVDHIVPQSFLKDDSIDNKVLTRSDKNRGKSDNVPSEEVVKKMKNYWRQLLNAKLITQRKEDNLTKAERGGLSELD |
| | | KAGFIKRQLVETRQITKHVAQILDSRMNTKYDENDKLIREVKVITLKSKLVSDERKDFQFYKVREINNYHHAHDAYLNAVVGTAL |
| | | IKKYPKLESEFVYGDYKVYDVRKMIAKSEQEIGKATAKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKKYGGEDSPTVAYSVLVVAKVEKGKSKKLKSVKELLGIT |
| | | IMERSSFEKNPIDFLEAKGYKEVKKDLIIKLPKYSLFELENGRKRMLASAGELQKGNELALPSKYVNFLYLASHYEKLKGSPEDN |
| | | EQKQLFVEQHKHYLDEIIEQISEFSKRVILADANLDKVLSAYNKHRDKPIREQAENIIHLFTLTNLGAPAAFKYEDTTIDRKRYT |
| | | STKEVLDATLIHQSITGLYETRIDLSQLGGDSGGSGGSGGSTNLSDIIEKETGKQLVIQESILMLPEEVEEVIGNKPESDILVHT |
| | | AYDESTDENVMLLTSDAPEYKPWALVIQDSNGENKIKMLSGGSGGSGGSTNLSDIIEKETGKQLVIQESILMLPEEVEEVIGNKP |
| | | ESDILVHTAYDESTDENVMLLTSDAPEYKPWALVIQDSNGENKIKMLSGGSKRTADGSEFEPKKKRKV** |
| CD30 Construct (DNA) | 981 | ATGGACTTCCAGGTGCAGATCTTCAGCTTCCTGCTGATCAGCGCCAGCGTGATCATGAGCCGGATGGCCCAGGTGCAGCTGCAGC |
| | | AGAGCGGCGCCGAGCTGGCCCGGCCCGGCGCCAGCGTGAAGATGAGCTGCAAGGCCAGCGGCTACACCTTCACCACCTACACCAT |
| | | CCACTGGGTGCGGCGGCGGCCCGGCCACGACCTGGAGTGGATCGGCTACATCAACCCCAGCAGCGGCTGCAGCGACTACAACCAG |
| | | AACTTCAAGGGCAAGACCACCCTGACCGCCGACAAGAGCAGCAACACCGCCTACATGCAGCTGAACAGCCTGACCAGCGAGGACA |
| | | GCGCCGTGTACTACTGCGCCCGGCGGGCCGACTACGGCAACTACGAGTACACCTGGTTCGCCTACTGGGGCCAGGGCACCACCGT |
| | | GACCGTGAGCAGCAGCGGCGGCGGCAGCGGCGGCGGCGGCAGCGGCGGCGGCGGCAGCGTGATCGAGCTGACCCAGAGCCCCAAG |
| | | TTCATGAGCACCAGCGTGGGCGACCGGGTGAACGTGACCTACAAGGCCAGCCAGAACGTGGGCACCAACGTGGCCTGGTTCCAGC |
| | | AGAAGCCCGGCCAGAGCCCCAAGGTGCTGATCTACAGCGCCAGCTACCGGTACAGCGGCGTGCCCGACCGGTTCACCGGCAGCGG |
| | | CAGCGGCACCGACTTCACCCTGACCATCAGCAACGTGCAGAGCGAGGACCTGGCCGAGTACTTCTGCCAGCAGTACCACACCTAC |
| | | CCCCTGACCTTCGGCGGCGGCACCAAGCTGGAGATCAAGCGGAGCGACCCCGCCACCACCACCCCCGCCCCCCGGCCCCCCACCC |
| | | CCGCCCCCACCATCGCCAGCCAGCCCCTGAGCCTGCGGCCCGAGGCCTGCCGGCCCGCCGCCGGCGGCGCCGTGCACACCCGGGG |
| | | CCTGGACTTCGCCTGCGACAAGGACCCCAAGTTCTGGGTGCTGGTGGTGGTGGGCGGCGTGCTGGCCTGCTACAGCCTGCTGGTG |
| | | ACCGTGGCCTTCATCATCTTCTGGGTGCGGAGCAAGCGGAGCCGGCTGCTGCACAGCGACTACATGAACATGACCCCCCGGCGGC |
| | | CCGGCCCCACCCGGAAGCACTACCAGCCCTACGCCCCCCCCCGGGACTTCGCCGCCTACCGGAGCCTGCGGGTGAAGTTCAGCCG |
| | | GAGCGCCGACGCCCCCGCCTACCAGCAGGGCCAGAACCAGCTGTACAACGAGCTGAACCTGGGCCGGCGGGAGGAGTACGACGTG |
| | | CTGGACAAGCGGCGGGGCCGGGACCCCGAGATGGGCGGCAAGCCCCGGCGGAAGAACCCCCAGGAGGGCCTGTACAACGAGCTGC |
| | | AGAAGGACAAGATGGCCGAGGCCTACAGCGAGATCGGCATGAAGGGCGAGCGGCGGCGGGGCAAGGGCCACGACGGCCTGTACCA |
| | | GGGCCTGAGCACCGCCACCAAGGACACCTACGACGCCCTGCACATGCAGGCCCTGCCCCCCCGGTAA |
| CD30 Construct (Amino | 982 | MDFQVQIFSFLLISASVIMSRMAQVQLQQSGAELARPGASVKMSCKASGYTFTTYTIHWVRRRPGHDLEWIGYINPSSGCSDYNQ |
| Acid) | | NFKGKTTLTADKSSNTAYMQLNSLTSEDSAVYYCARRADYGNYEYTWFAYWGQGTTVTVSSSGGGSGGGGSGGGGSVIELTQSPK |
| | | FMSTSVGDRVNVTYKASQNVGTNVAWFQQKPGQSPKVLIYSASYRYSGVPDRFTGSGSGTDFTLTISNVQSEDLAEYFCQQYHTY |
| | | PLTFGGGTKLEIKRSDPATTTPAPRPPTPAPTIASQPLSLRPEACRPAAGGAVHTRGLDFACDKDPKFWVLVVVGGVLACYSLLV |
| | | TVAFIIFWVRSKRSRLLHSDYMNMTPRRPGPTRKHYQPYAPPRDFAAYRSLRVKFSRSADAPAYQQGQNQLYNELNLGRREEYDV |
| | | LDKRRGRDPEMGGKPRRKNPQEGLYNELQKDKMAEAYSEIGMKGERRRGKGHDGLYQGLSTATKDTYDALHMQALPPR* |
| CD30 Construct Signal | 983 | ATGGACTTCCAGGTGCAGATCTTCAGCTTCCTGCTGATCAGCGCCAGCGTGATCATGAGCCGGATGGCC |
| Peptide (DNA) | | |
| CD30 Construct Signal | 984 | MDFQVQIFSELLISASVIMSRMA |
| Peptide (Amino Acid) | | |
| CD30 Construct HRS3 | 985 | CAGGTGCAGCTGCAGCAGAGCGGCGCCGAGCTGGCCCGGCCCGGCGCCAGCGTGAAGATGAGCTGCAAGGCCAGCGGCTACACCT |
| Binder (DNA) | | TCACCACCTACACCATCCACTGGGTGCGGCGGCGGCCCGGCCACGACCTGGAGTGGATCGGCTACATCAACCCCAGCAGCGGCTG |
| | | CAGCGACTACAACCAGAACTTCAAGGGCAAGACCACCCTGACCGCCGACAAGAGCAGCAACACCGCCTACATGCAGCTGAACAGC |
| | | CTGACCAGCGAGGACAGCGCCGTGTACTACTGCGCCCGGCGGGCCGACTACGGCAACTACGAGTACACCTGGTTCGCCTACTGGG |
| | | GCCAGGGCACCACCGTGACCGTGAGCAGCAGCGGCGGCGGCAGCGGCGGCGGCGGCAGCGGCGGCGGCGGCAGCGTGATCGAGCT |
| | | GACCCAGAGCCCCAAGTTCATGAGCACCAGCGTGGGCGACCGGGTGAACGTGACCTACAAGGCCAGCCAGAACGTGGGCACCAAC |
| | | GTGGCCTGGTTCCAGCAGAAGCCCGGCCAGAGCCCCAAGGTGCTGATCTACAGCGCCAGCTACCGGTACAGCGGCGTGCCCGACC |
| | | GGTTCACCGGCAGCGGCAGCGGCACCGACTTCACCCTGACCATCAGCAACGTGCAGAGCGAGGACCTGGCCGAGTACTTCTGCCA |
| | | GCAGTACCACACCTACCCCCTGACCTTCGGCGGCGGCACCAAGCTGGAGATCAAGCGG |
| CD30 Construct HRS3 | 986 | QVQLQQSGAELARPGASVKMSCKASGYTFTTYTIHWVRRRPGHDLEWIGYINPSSGCSDYNQNEKGKTTLTADKSSNTAYMQLNS |
| Binder (Amino Acid) | | LTSEDSAVYYCARRADYGNYEYTWFAYWGQGTTVTVSSSGGGSGGGGSGGGGSVIELTQSPKFMSTSVGDRVNVTYKASQNVGTN |
| | | VAWFQQKPGQSPKVLIYSASYRYSGVPDRFTGSGSGTDFTLTISNVQSEDLAEYFCQQYHTYPLTFGGGTKLEIKR |
| CD30 Construct CD8 | 987 | ACCACCACCCCCGCCCCCCGGCCCCCCACCCCCGCCCCCACCATCGCCAGCCAGCCCCTGAGCCTGCGGCCCGAGGCCTGCCGGC |
| Hinge (DNA) | | CCGCCGCCGGCGGCGCCGTGCACACCCGGGGCCTGGACTTCGCCTGCGAC |
| CD30 Construct CD8 | 988 | TTTPAPRPPTPAPTIASQPLSLRPEACRPAAGGAVHTRGLDFACD |
| Hinge (Amino Acid) | | |
| CD30 Construct CD28 | 989 | TTCTGGGTGCTGGTGGTGGTGGGCGGCGTGCTGGCCTGCTACAGCCTGCTGGTGACCGTGGCCTTCATCATCTTCTGGGTGCGGA |
| TM (DNA) | | GCAAGCGGAGCCGGCTGCTGCACAGCGACTACATGAACATGACCCCCCGGCGGCCCGGCCCCACCCGGAAGCACTACCAGCCCTA |
| | | CGCCCCCCCCCGGGACTTCGCCGCCTACCGGAGC |
| CD30 Construct CD28 | 990 | FWVLVVVGGVLACYSLLVTVAFIIFWVRSKRSRLLHSDYMNMTPRRPGPTRKHYQPYAPPRDFAAYRS |
| TM (Amino Acid) | | |
| CD30 Construct | 991 | CTGCGGGTGAAGTTCAGCCGGAGCGCCGACGCCCCCGCCTACCAGCAGGGCCAGAACCAGCTGTACAACGAGCTGAACCTGGGCC |
| CD3zeta (DNA) | | GGCGGGAGGAGTACGACGTGCTGGACAAGCGGCGGGGCCGGGACCCCGAGATGGGCGGCAAGCCCCGGCGGAAGAACCCCCAGGA |
| | | GGGCCTGTACAACGAGCTGCAGAAGGACAAGATGGCCGAGGCCTACAGCGAGATCGGCATGAAGGGCGAGCGGGGGGGGCAAG |
| | | GGCCACGACGGCCTGTACCAGGGCCTGAGCACCGCCACCAAGGACACCTACGACGCCCTGCACATGCAGGCCCTGCCCCCCCGG |
| CD30 Construct | 992 | LRVKFSRSADAPAYQQGQNQLYNELNLGRREEYDVLDKRRGRDPEMGGKPRRKNPQEGLYNELQKDKMAEAYSEIGMKGERRRGK |
| CD3zeta (Amino Acid) | | GHDGLYQGLSTATKDTYDALHMQALPPR |
| B2M (G000529) | 993 | mG*mG*mC*CACGGAGCGAGACAUCUGUUUUAGAmGmCmUmAmGmAmAmAmUmAmGmCAAGUUAAAAUAAGGCUAGUCCGUUAUC |
| | | AmAmCmUmUmGmAmAmAmAmAmGmUmGmGmCmAmCmCmGmAmGmUmCmGmGmUmGmCmU*mU*mU*mU |
| G021469 TRAC | 994 | mA*mU*mA*mUmCCAmGmAAmCCmCUGACmCCUGmCCGmGUUGmUmAmGmCUCCCmUmGmAmAmAmCmCGUUmGmCUAmCAAU*A |
| | | AGmGmCCmGmUmCmGmAmAmAmGmAmUGUGCmCGmCAAmCGCUCUmGmCCmUmUmCmUGGCAUCG*mU*mU |
| G021475 TRAC | 995 | mA*mA*mC*mCmCUGmAmUCmCUmCUUGUmCCCAmCAGmGUUGmUmAmGmCUCCCmUmGmAmAmAmCmCGUUmGmCUAmCAAU*A |
| | | AGmGmCCmGmUmCmGmAmAmAmGmAmUGUGCmCGmCAAmCGCUCUmGmCCmUmUmCmUGGCAUCG*mU*mU |
| G021481 TRAC | 996 | mG*mC*mC*mGmUGUmAmCCmAGmCUGAGmAGACmUCUmGUUGmUmAmGmCUCCCmUmGmAmAmAmCmCGUUmGmCUAmCAAU*A |
| | | AGmGmCCmGmUmCmGmAmAmAmGmAmUGUGCmCGmCAAmCGCUCUmGmCCmUmUmCmUGGCAUCG*mU*mU |
| G028013 B2M | 997 | mG*mG*mC*CACGGAGCGAGACAUCUGUUUUAGAmGmCmUmAmGmAmAmAmUmAmGmCAAGUUAAAAUAAGGCUAGUCCGUUAUC |
| | | AmAmCmUmUmGmAmAmAmAmAmGmUmGmGmCmAmCmCmGmAmGmUmCmGmGmUmGmCmU*mU*mU*mU |
| G018995 HLA-A | 998 | mA*mC*mA*GCGACGCCGCGAGCCAGGUUUUAGAmGmCmUmAmGmAmAmAmUmAmGmCAAGUUAAAAUAAGGCUAGUCCGUUAUC |
| | | AmAmCmUmUmGmAmAmAmAmAmGmUmGmGmCmAmCmCmGmAmGmUmCmGmGmUmGmCmU*mU*mU*mU |
| G012086 TRAC | 999 | mA*mG*mA*GUCUCUCAGCUGGUACAGUUUUAGAmGmCmUmAmGmAmAmAmUmAmGmCAAGUUAAAAUAAGGCUAGUCCGUUAUC |
| | | AmAmCmUmUmGmAmAmAmAmAmGmUmGmGmCmAmCmCmGmAmGmUmCmGmGmUmGmCmU*mU*mU*mU |
| Guide Scaffold | 3002 | NNNNNNNNNNNNNNNNNNNNGUUUUAGAGCUAGAAAUAGCAAGUUAAAAUAAGGCUAGUCCGUUAUCACGAAAGGGCACCGAGUC |
| | | GGUGCU |
| Guide scaffold | 3003 | mN*mN*mN*NNNNNNNNNNNNNNNNNGUUUUAGAmGmCmUmAmGmAmAmAmUmAmGmCAAGUUAAAAUAAGGCUAGUCCGUUAUC |
| | | ACGAAAGGGCACCGAGUCGGmUmGmC*mU |
| | 3004 | Not Used |
| mRNA sequence | 3005 | GGGAAGCUCAGAAUAAACGCUCAACUUUGGCCGGAUCUGCCACCAUGACCAACCUGUCCGACAUCAUCGAGAAGGAGACCGGCAA |
| encoding UGI | | GCAGCUGGUGAUCCAGGAGUCCAUCCUGAUGCUGCCCGAGGAGGUGGAGGAGGUGAUCGGCAACAAGCCCGAGUCCGACAUCCUG |
| | | GUGCACACCGCCUACGACGAGUCCACCGACGAGAACGUGAUGCUGCUGACCUCCGACGCCCCCGAGUACAAGCCCUGGGCCCUGG |
| | | UGAUCCAGGACUCCAACGGCGAGAACAAGAUCAAGAUGCUGUCCGGCGGCUCCAAGCGGACCGCCGACGGCUCCGAGUUCGAGUC |
| | | CCCCAAGAAGAAGCGGAAGGUGGAGUGAUAGCUAGCACCAGCCUCAAGAACACCCGAAUGGAGUCUCUAAGCUACAUAAUACCAA |
| | | CUUACACUUUACAAAAUGUUGUCCCCCAAAAUGUAGCCAUUCGUAUCUGCUCCUAAUAAAAAGAAAGUUUCUUCACAUUCUCUCG |
| | | AGAAAAAAAAAAAAUGGAAAAAAAAAAAACGGAAAAAAAAAAAAGGUAAAAAAAAAAAAUAUAAAAAAAAAAAACAUAAAAAAAA |
| | | AAAACGAAAAAAAAAAAACGUAAAAAAAAAAAACUCAAAAAAAAAAAAGAUAAAAAAAAAAAACCUAAAAAAAAAAAAUGUAAAA |
| | | AAAAAAAAGGGAAAAAAAAAAAACGCAAAAAAAAAAAACACAAAAAAAAAAAAUGCAAAAAAAAAAAAUCGAAAAAAAAAAAAUC |
| | | UAAAAAAAAAAAACGAAAAAAAAAAAACCCAAAAAAAAAAAAGACAAAAAAAAAAAAUAGAAAAAAAAAAAAGUUAAAAAAAAAA |
| | | AACUGAAAAAAAAAAAAUUUAAAAAAAAAAAAUCUAG |
| Guide scaffold 90-mer | 3006 | GUUUUAGAGCUAGAAAUAGCAAGUUAAAAUAAGGCUAGUCCGUUAUCACGAAAGGGCACCGAGUCGGUGC |
| Guide scaffold 90-mer | 3007 | GUUUUAGAmGmCmUmAmGmAmAmAmUmAmGmCAAGUUAAAAUAAGGCUAGUCCGUUAUCACGAAAGGGCACCGAGUCGG*mU*mG |
| with modification | | *mC |
| Guide scaffold 90-mer | 3008 | GUUUUAGAmGmCmUmAmGmAmAmAmUmAmGmCAAGUUAAAAUAAGGCUAGUCCGUUAUCAmCmGmAmAmAmGmGmGmCmAmCmCm |
| with modification | | GmAmGmUmCmGmG*mU*mG*mC |
| Guide scaffold 88-mer | 3009 | GUUUUAGAmGmCmUmAmGmAmAmAmUmAmGmCAAGUUAAAAUAAGGCUAGUCCGUUAUCAACUUGGCACCGAGUCGG*mU*mG*m |
| with modification | | C |
| Guide scaffold 88-mer | 3010 | GUUUUAGAGCUAGAAAUAGCAAGUUAAAAUAAGGCUAGUCCGUUAUCAAAAUGGCACCGAGUCGGUGC |
| Guide scaffold 88-mer | 3011 | GUUUUAGAmGmCmUmAmGmAmAmAmUmAmGmCAAGUUAAAAUAAGGCUAGUCCGUUAUCAAAAUGGCACCGAGUCGG*mU*mG*m |
| with modification | | C |
| Guide scaffold 88-mer | 3012 | GUUUUAGAmGmCmUmAmGmAmAmAmUmAmGmCAAGUUAAAAUAAGGCUAGUCCGUUAUCAmAmAmAmUmGmGmCmAmCmCmGmAm |
| with modification | | GmUmCmGmG*mU*mG*mC |
| Guide scaffold | 3013 | GUUUUAGAmGmCmUmAmGmAmAmAmUmAmGmCAAGUUAAAAUAAGGCUAGUCCGUUAUCAmAmCmUmUmGmAmAmAmAmAmGmUm |
| | | GmGmCmAmCmCmGmAmGmUmCmGmGmUmGmCmU*mU*mU*mU |
| Guide scaffold | 3014 | mN*mN*mN*NNNNNNNNNNNNNNNNNGUUUUAGAmGmCmUmAmGmAmAmAmUmAmGmCAAGUUAAAAUAAGGCUAGUCCGUUAUC |
| | | AmAmCmUmUmGmAmAmAmAmAmGmUmGmGmCmAmCmCmGmAmGmUmCmGmGmUmGmCmU*mU*mU*mU |
| | 3015-3018 | Not Used |
| G025420 TRAC | 3019 | mC*mU*mC*UCAGCUGGUACACGGCAGUUUUAGAmGmCmUmAmGmAmAmAmUmAmGmCAAGUUAAAAUAAGGCUAGUCCGUUAUC |
| | | ACGAAAGGGCACCGAGUCGGmU*mG*mC*mU |
| B2M (G000529) (full, | 3100 | GGCCACGGAGCGAGACAUCUGUUUUAGAGCUAGAAAUAGCAAGUUAAAAUAAGGCUAGUCCGUUAUCAACUUGAAAAAGUGGCAC |
| unmodified) | | CGAGUCGGUGCUUUU |
| G021469 TRAC (full, | 3101 | AUAUCCAGAACCCUGACCCUGCCGGUUGUAGCUCCCUGAAACCGUUGCUACAAUAAGGCCGUCGAAAGAUGUGCCGCAACGCUCU |
| unmodified) | | GCCUUCUGGCAUCGUU |
| G021475 TRAC (full, | 3102 | AACCCUGAUCCUCUUGUCCCACAGGUUGUAGCUCCCUGAAACCGUUGCUACAAUAAGGCCGUCGAAAGAUGUGCCGCAACGCUCU |
| unmodified) | | GCCUUCUGGCAUCGUU |
| G021481 TRAC (full, | 3103 | GCCGUGUACCAGCUGAGAGACUCUGUUGUAGCUCCCUGAAACCGUUGCUACAAUAAGGCCGUCGAAAGAUGUGCCGCAACGCUCU |
| unmodified) | | GCCUUCUGGCAUCGUU |
| G028013 B2M (full, | 3104 | GGCCACGGAGCGAGACAUCUGUUUUAGAGCUAGAAAUAGCAAGUUAAAAUAAGGCUAGUCCGUUAUCAACUUGAAAAAGUGGCAC |
| unmodified) | | CGAGUCGGUGCUUUU |
| G018995 HLA-A (full, | 3105 | ACAGCGACGCCGCGAGCCAGGUUUUAGAGCUAGAAAUAGCAAGUUAAAAUAAGGCUAGUCCGUUAUCAACUUGAAAAAGUGGCAC |
| unmodified) | | CGAGUCGGUGCUUUU |
| G012086 TRAC (full, | 3106 | AGAGUCUCUCAGCUGGUACAGUUUUAGAGCUAGAAAUAGCAAGUUAAAAUAAGGCUAGUCCGUUAUCAACUUGAAAAAGUGGCAC |
| unmodified) | | CGAGUCGGUGCUUUU |
| G000644 EMX1 target | 3107 | GAGUCCGAGCAGAAGAAGAA |
| sequence | | |
| G000645 VEGFA target | 3108 | GACCCCCUCCACCCCGCCUC |
| sequence | | |
| G000646 RAG1B target | 3109 | GACUUGUUUUCAUUGUUCUC |
| sequence | | |
| G013675 CIITA target | 3110 | CCCCCGGACGGUUCAAGCAA |
| sequence | | |
| G013675 CIITA sgRNA | 3118 | mC*mC*mC*CCGGACGGUUCAAGCAAGUUUUAGAmGmCmUmAmGmAmAmAmUmAmGmCAAGUUAAAAUAAGGCUAGUCCGUUAUC |
| | | AmAmCmUmUmGmAmAmAmAmAmGmUmGmGmCmAmCmCmGmAmGmUmCmGmGmUmGmCmU*mU*mU*mU |

TABLE 9A
VII. Additional Exemplary Nme Guide RNAs

| | | | | | Genomic |
|---|---|---|---|---|---|
| Guide | | Guide | | | Coordinates |
| ID | Target | Sequence | Exemplary Guide RNA Full Sequence | Exemplary Guide RNA Modified Sequence | (hg38) |
| G034202 | HLA-A | GCUCUAU | GCUCUAUCCACGGCGCCCGCGGCUGUUGU | mG*mC*mU*mCmUAUmCmCAmCGmGCGCCmCGCGmGCUmGUUGmUm | chr6: |
| | | CCACGGC | AGCUCCCUGAAACCGUUGCUACAAUAAGG | AmGmCUCCCmUmGmAmAmAmCmCGUUmGmCUAmCAAUAAGmGmCC | 29942891- |
| | | GCCCGCG | CCGUCGAAAGAUGUGCCGCAACGCUCUGC | mGmUmCmGmAmAmAmGmAmUGUGCmCGmCAAmCGCUCUmGmCCmU | 29942915 |
| | | GCU (SEQ | CUUCUGGCAUCGUU (SEQ ID NO: 1576) | mUmCmUGGCAUCG*mU*mU (SEQ ID NO: 3111) | |
| | | ID NO: | | | |
| | | 576) | | | |
| G034617 | HLA-A | CACUCAC | CACUCACCCGCCCAGGUCUGGGUCGUUGU | mC*mA*mC*mUmCACmCmCGmCCmCAGGUmCUGGmGUCmGUUGmUm | chr6: |
| | | CCGCCCA | AGCUCCCUGAAACCGUUGCUACAAUAAGG | AmGmCUCCCmUmGmAmAmAmCmCGUUmGmCUAmCAAUAAGmGmCC | 29942609- |
| | | GGUCUGG | CCGUCGAAAGAUGUGCCGCAACGCUCUGC | mGmUmCmGmAmAmAmGmAmUGUGCmCGmCAAmCGCUCUmGmCCmU | 29942633 |
| | | GUC (SEQ | CUUCUGGCAUCGUU (SEQ ID NO: 1571) | mUmCmUGGCAUCG*mU*mU (SEQ ID NO: 3112) | |
| | | ID NO: | | | |
| | | 571) | | | |
| G028943 | TRAC | AAAACCU | AAAACCUGUCAGUGAUUGGGUUCCGUUGU | mA*mA*mA*mAmCCUmGmUCmAGmUGAUUmGGGUmUCCmGUUGmUm | chr14: |
| | | GUCAGUG | AGCUCCCUGAAACCGUUGCUACAAUAAGG | AmGmCUCCCmUmGmAmAmAmCmCGUUmGmCUAmCAAU*AAGmGmC | 22550574- |
| | | AUUGGG | CCGUCGAAAGAUGUGCCGCAACGCUCUGC | CmGmUmCmGmAmAmAmGmAmUGUGCmCGmCAAmCGCUCUmGmCCm | 22550598 |
| | | UUCC | CUUCUGGCAUCGUU (SEQ ID NO: 1605) | UmUmCmUGGCAUCG*mU*mU (SEQ ID NO: 2605) | |
| | | (SEQ ID | | | |
| | | NO: 605) | | | |
| G034982 | TRAC | AAAACCU | AAAACCUGUCAGUGAUUGGGUUCCGUUGU | mA*mA*mA*mAmCCUmGmUCmAGmUGAUUmGGGUmUCCmGUUGmUm | chr14: |
| | | GUCAGUG | AGCUCCCUGAAACCGUUGCUACAAUAAGG | AmGmCUCCCmUmGmAmAmAmCmCGUUmGmCUAmCAAUAAGmGmCC | 22550574- |
| | | AUUGGG | CCGUCGAAAGAUGUGCCGCAACGCUCUGC | mGmUmCmGmAmAmAmGmAmUGUGCmCGmCAAmCGCUCUmGmCCmU | 22550598 |
| | | UUCC | CUUCUGGCAUCGUU (SEQ ID NO: 1605) | mUmCmUGGCAUCG*mU*mU (SEQ ID NO: 3113) | |
| | | (SEQ ID | | | |
| | | NO: 605) | | | |
| G028939 | TRAC | UUAGGU | UUAGGUUCGUAUCUGUAAAACCAAGUUGU | mU*mU*mA*mGmGUUmCmGUmAUmCUGUAmAAACmCAAmGUUGmU | chr14: |
| | | UCGUAUC | AGCUCCCUGAAACCGUUGCUACAAUAAGG | mAmGmCUCCCmUmGmAmAmAmCmCGUUmGmCUAmCAAU*AAGmGm | 22550544- |
| | | UGUAAA | CCGUCGAAAGAUGUGCCGCAACGCUCUGC | CCmGmUmCmGmAmAmAmGmAmUGUGCmCGmCAAmCGCUCUmGmCC | 22550568 |
| | | ACCAA | CUUCUGGCAUCGUU (SEQ ID NO: 1606) | mUmUmCmUGGCAUCG*mU*mU (SEQ ID NO: 2606) | |
| | | (SEQ ID | | | |
| | | NO: 606) | | | |
| G034981 | TRAC | UUAGGU | UUAGGUUCGUAUCUGUAAAACCAAGUUGU | mU*mU*mA*mGmGUUmCmGUmAUmCUGUAmAAACmCAAmGUUGmU | chr14: |
| | | UCGUAUC | AGCUCCCUGAAACCGUUGCUACAAUAAGG | mAmGmCUCCCmUmGmAmAmAmCmCGUUmGmCUAmCAAUAAGmGmC | 22550544- |
| | | UGUAAA | CCGUCGAAAGAUGUGCCGCAACGCUCUGC | CmGmUmCmGmAmAmAmGmAmUGUGCmCGmCAAmCGCUCUmGmCCm | 22550568 |
| | | ACCAA | CUUCUGGCAUCGUU (SEQ ID NO: 1606) | UmUmCmUGGCAUCG*mU*mU (SEQ ID NO: 3114) | |
| | | (SEQ ID | | | |
| | | NO: 606) | | | |
| G013006 | TRAC | CUCUCAG | CUCUCAGCUGGUACACGGCAGUUUUAGAG | mC*mU*mC*UCAGCUGGUACACGGCAGUUUUAGAmGmCmUmAmGmA | chr14: |
| | | CUGGUAC | CUAGAAAUAGCAAGUUAAAAUAAGGCUAG | mAmAmUmAmGmCAAGUUAAAAUAAGGCUAGUCCGUUAUCAmAmCm | 22547524- |
| | | ACGGCA | UCCGUUAUCAACUUGAAAAAGUGGCACCG | UmUmGmAmAmAmAmAmGmUmGmGmCmAmCmCmGmAmGmUmCmGm | 22547544 |
| | | (SEQ ID | AGUCGGUGCUUUU (SEQ ID NO: 1613) | GmUmGmCmU*mU*mU*mU (SEQ ID NO: 2613) | |
| | | NO: 613) | | | |
| G028986 | TRBC1 | GUGUCCU | GUGUCCUACCAGCAAGGGGUCCUGGUUGU | mG*mU*mG*mUmCCUmAmCCmAGmCAAGGmGGUCmCUGmGUUGmUm | chr7: |
| | | ACCAGCA | AGCUCCCUGAAACCGUUGCUACAAUAAGG | AmGmCUCCCmUmGmAmAmAmCmCGUUmGmCUAmCAAU*AAGmGmC | 142792690- |
| | | AGGGGUC | CCGUCGAAAGAUGUGCCGCAACGCUCUGC | CmGmUmCmGmAmAmAmGmAmUGUGCmCGmCAAmCGCUCUmGmCCm | 142792714 |
| | | CUG (SEQ | CUUCUGGCAUCGUU (SEQ ID NO: 1607) | UmUmCmUGGCAUCG*mU*mU (SEQ ID NO: 2607) | |
| | | ID NO: | | | |
| | | 607) | | | |
| G034618 | TRBC1 | GUGUCCU | GUGUCCUACCAGCAAGGGGUCCUGGUUGU | mG*mU*mG*mUmCCUmAmCCmAGmCAAGGmGGUCmCUGmGUUGmUm | chr7: |
| | | ACCAGCA | AGCUCCCUGAAACCGUUGCUACAAUAAGG | AmGmCUCCCmUmGmAmAmAmCmCGUUmGmCUAmCAAUAAGmGmCC | 142792690- |
| | | AGGGGUC | CCGUCGAAAGAUGUGCCGCAACGCUCUGC | mGmUmCmGmAmAmAmGmAmUGUGCmCGmCAAmCGCUCUmGmCCmU | 142792714 |
| | | CUG (SEQ | CUUCUGGCAUCGUU (SEQ ID NO: 1607) | mUmCmUGGCAUCG*mU*mU (SEQ ID NO: 3115) | |
| | | ID NO: | | | |
| | | 607) | | | |
| G026584 | CIITA | UCAAAGU | UCAAAGUACCCUACAGGAGGACCAGUUGU | mU*mC*mA*mAmAGUmAmCCmCUmACAGGmAGGAmCCAmGUUGmUm | chr16: |
| | | ACCCUAC | AGCUCCCUGAAACCGUUGCUACAAUAAGG | AmGmCUCCCmUmGmAmAmAmCmCGUUmGmCUAmCAAU*AAGmGmC | 10907504- |
| | | AGGAGG | CCGUCGAAAGAUGUGCCGCAACGCUCUGC | CmGmUmCmGmAmAmAmGmAmUGUGCmCGmCAAmCGCUCUmGmCCm | 10907528 |
| | | ACCA | CUUCUGGCAUCGUU (SEQ ID NO: 1608) | UmUmCmUGGCAUCG*mU*mU (SEQ ID NO: 2608) | |
| | | (SEQ ID | | | |
| | | NO: 608) | | | |
| G034201 | CIITA | UCAAAGU | UCAAAGUACCCUACAGGAGGACCAGUUGU | mU*mC*mA*mAmAGUmAmCCmCUmACAGGmAGGAmCCAmGUUGmUm | chr16: |
| | | ACCCUAC | AGCUCCCUGAAACCGUUGCUACAAUAAGG | AmGmCUCCCmUmGmAmAmAmCmCGUUmGmCUAmCAAUAAGmGmCC | 10907504- |
| | | AGGAGG | CCGUCGAAAGAUGUGCCGCAACGCUCUGC | mGmUmCmGmAmAmAmGmAmUGUGCmCGmCAAmCGCUCUmGmCCmU | 10907528 |
| | | ACCA | CUUCUGGCAUCGUU (SEQ ID NO: 1608) | mUmCmUGGCAUCG*mU*mU (SEQ ID NO: 3116) | |
| | | (SEQ ID | | | |
| | | NO: 608) | | | |
| G029131 | CIITA | AGCUGCC | AGCUGCCGUUCUGCCCAGUCCGGGGUUGU | mA*mG*mC*mUmGCCmGmUUmCUmGCCCAmGUCCmGGGmGUUGmUm | chr16: |
| | | GUUCUGC | AGCUCCCUGAAACCGUUGCUACAAUAAGG | AmGmCUCCCmUmGmAmAmAmCmCGUUmGmCUAmCAAU*AAGmGmC | 10906643- |
| | | CCAGUCC | CCGUCGAAAGAUGUGCCGCAACGCUCUGC | CmGmUmCmGmAmAmAmGmAmUGUGCmCGmCAAmCGCUCUmGmCCm | 10906667 |
| | | GGG (SEQ | CUUCUGGCAUCGUU (SEQ ID NO: 1609) | UmUmCmUGGCAUCG*mU*mU (SEQ ID NO: 2609) | |
| | | ID NO: | | | |
| | | 609) | | | |
| G034619 | CIITA | AGCUGCC | AGCUGCCGUUCUGCCCAGUCCGGGGUUGU | mA*mG*mC*mUmGCCmGmUUmCUmGCCCAmGUCCmGGGmGUUGmUm | chr16: |
| | | GUUCUGC | AGCUCCCUGAAACCGUUGCUACAAUAAGG | AmGmCUCCCmUmGmAmAmAmCmCGUUmGmCUAmCAAUAAGmGmCC | 10906643- |
| | | CCAGUCC | CCGUCGAAAGAUGUGCCGCAACGCUCUGC | mGmUmCmGmAmAmAmGmAmUGUGCmCGmCAAmCGCUCUmGmCCmU | 10906667 |
| | | GGG (SEQ | CUUCUGGCAUCGUU (SEQ ID NO: 1609) | mUmCmUGGCAUCG*mU*mU (SEQ ID NO: 3117) | |
| | | ID NO: | | | |
| | | 609) | | | |
| G021557 | VEGFA | GCAUGGG | GCAUGGGCAGGGGCUGGGGUGCACGUUGU | mG*mC*mA*mUmGGGmCmAGmGGmGCUGGmGGUGmCACmGUUGmUm | chr6: |
| | | CAGGGGC | AGCUCCCUGAAACCGUUGCUACAAUAAGG | AmGmCUCCCmUmGmAmAmAmCmCGUUmGmCUAmCAAU*AAGmGmC | 43774288- |
| | | UGGGGU | CCGUCGAAAGAUGUGCCGCAACGCUCUGC | CmGmUmCmGmAmAmAmGmAmUGUGCmCGmCAAmCGCUCUmGmCCm | 43774312 |
| | | GCAC | CUUCUGGCAUCGUU (SEQ ID NO: 1610) | UmUmCmUGGCAUCG*mU*mU (SEQ ID NO: 2610) | |
| | | (SEQ ID | | | |
| | | NO: 610) | | | |
| G021558 | VEGFA | GAAUGGC | GAAUGGCAGGCGGAGGUUGUACUGGUUGU | mG*mA*mA*mUmGGCmAmGGmCGmGAGGUmUGUAmCUGmGUUGmU | chr6: |
| | | AGGCGGA | AGCUCCCUGAAACCGUUGCUACAAUAAGG | mAmGmCUCCCmUmGmAmAmAmCmCGUUmGmCUAmCAAU*AAGmGm | 43780852- |
| | | GGUUGU | CCGUCGAAAGAUGUGCCGCAACGCUCUGC | CCmGmUmCmGmAmAmAmGmAmUGUGCmCGmCAAmCGCUCUmGmCC | 43780876 |
| | | ACUG | CUUCUGGCAUCGUU (SEQ ID NO: 1611) | mUmUmCmUGGCAUCG*mU*mU (SEQ ID NO: 2611) | |
| | | (SEQ ID | | | |
| | | NO: 611) | | | |
| G021567 | VEGFA | GUGAGCA | GUGAGCAGGCACCUGUGCCAACAUGUUGU | mG*mU*mG*mAmGCAmGmGCmACmCUGUGmCCAAmCAUmGUUGmUm | chr6: |
| | | GGCACCU | AGCUCCCUGAAACCGUUGCUACAAUAAGG | AmGmCUCCCmUmGmAmAmAmCmCGUUmGmCUAmCAAU*AAGmGmC | 43781113- |
| | | GUGCCAA | CCGUCGAAAGAUGUGCCGCAACGCUCUGC | CmGmUmCmGmAmAmAmGmAmUGUGCmCGmCAAmCGCUCUmGmCCm | 43781137 |
| | | CAU (SEQ | CUUCUGGCAUCGUU (SEQ ID NO: 1612) | UmUmCmUGGCAUCG*mU*mU (SEQ ID NO: 2612) | |
| | | ID NO: | | | |
| | | 612) | | | |

### VII. EXAMPLES

The following examples are provided to illustrate certain disclosed embodiments and are not to be construed as limiting the scope of this disclosure in any way.

### Example 1. General Methods

### 1.1. T Cell Culture Media Preparation

T cell culture media compositions used below are described here. “TCAM Media” comprises of CTS OpTimizer T Cell Expansion SFM containing 2.5% (v/v) of Human AB Serum, 1% (v/v) Glutamax, 1% (v/v) 10 1M HEPES buffer, and 1% of Penicillin-Streptomycin. In addition to the above mentioned components, media was supplemented with 100 U/mL of recombinant human interleukin-2, 5 ng/mL of human interleukin-7, and 5 ng/mL of human interleukin-15.

### 1.2. Preparation of Lipid Nanoparticles

The lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), Lipid nanoparticles used 35% Lipid A, 47.5% cholesterol, 15% DSPC, and 2.5% PEG2k-DMG by molarity. The lipid nucleic acid assemblies were formulated with a lipid amine to RNA phosphate (N:P) molar ratio of about 6, and a ratio of gRNA to mRNA of 1:2 by weight.

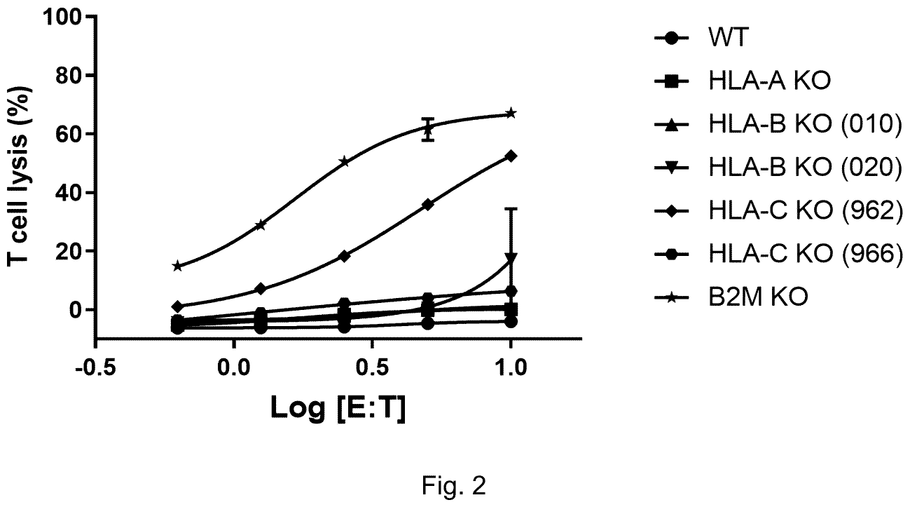

Lipid nanoparticles (LNP compositions) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2). The LNP compositions were held for 1 hour at room temperature (RT), and further diluted with water (approximately 1:1 v/v). LNP compositions were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

1.3. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation sequence was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating 50 ng/L linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer at 37° C. for 1.5-4 hours. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In an alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

S. pyogenes (“Spy”) Cas9 mRNA were generated from plasmid DNA encoding an open reading frame having a nucleic acid sequence of one of SEQ ID NOs: 801-803 and 806 (see sequences in Table 9). When SEQ ID NOs: 801-803 and 806 are referred to below with respect to RNAs, it is understood that Ts should be replaced with Us (which were N1-methyl pseudouridines as described above). Messenger RNAs used in the Examples include a 5′ cap and a 3′ polyadenylation region, e.g., up to 100 nucleotides, and have a nucleic acid sequence of one of SEQ ID NOs: 801-803 and 806 in Table 9.

### Example 2: Screening of HLA-B Guide RNAs with Spy Cas9

48 sgRNAs in the 100-nt modified sgRNA format designed for the disruption of the HLA-B gene were screened for efficacy in T cells by assessing loss of HLA-B surface protein. The donor had an HLA-B phenotype of B*07:02 and B*07:02. The percentage of T cells negative for HLA-B7 was determined by flow cytometry following editing at the HLA-B locus by electroporation with Cas9 ribonucleoprotein (RNP) and each test guide.

### 2.1. RNP Electroporation of T Cells

Cas9 editing activity was assessed using electroporation of Cas9 ribonucleoprotein (RNP). Upon thaw, Pan CD3+ T cells (StemCell, HLA-B*07:02/B*07:02) were plated at a density of 0.5×10\hat{ }6 cells/mL in T cell RPMI media composed of RPMI 1640 (Invitrogen, Cat. 22400-089) containing 5% (v/v) of fetal bovine serum, 1× Glutamax (Gibco, Cat. 35050-061), 50 μM of 2-Mercaptoethanol, 100 μM non-essential amino acids (Invitrogen, Cat. 11140-050), 1 mM sodium pyruvate, 10 mM HEPES buffer, 1% of Penicillin-Streptomycin, and 100 U/mL of recombinant human interleukin-2 (Peprotech, Cat. 200-02). T cells were activated with TransAct™ (1:100 dilution, Miltenyi Biotec). Cells were expanded in T cell RPMI media for 72 hours prior to RNP transfection.

HLA-B targeting sgRNAs and control B2M and HLA-A targeting sgRNAs were removed from their storage plates and denatured for 2 minutes at 95° C. before cooling at room temperature for 10 minutes. RNP mixture of 20 μM sgRNA and 10 μM Cas9-NLS protein (SEQ ID NO: 800) was prepared and incubated at 25° C. for 10 minutes. Five μL of RNP mixture was combined with 100,000 cells in 20 μL P3 electroporation Buffer (Lonza). 22 μL of RNP/cell mix was transferred to the corresponding wells of a Lonza shuttle 96-well electroporation plate. Cells were electroporated in duplicate with the manufacturer's pulse code. T cell RPMI media was added to the cells immediately post electroporation.

### 2.2. Flow Cytometry

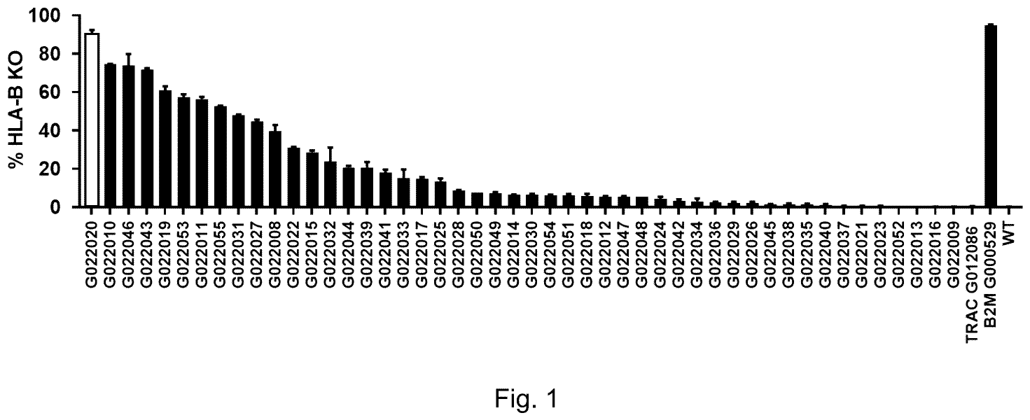

On day 7 post-edit, T cells were phenotyped by flow cytometry to determine HLA-B protein expression following editing at the HLA-B locus. Briefly, T cells were incubated in antibody targeting HLA-B7, B27 (Miltenyi, Clone REA176, 130-120-234) surface protein corresponding the cells donor's genotype (HLA-B*07:02/B*07:02). Cells were subsequently washed, processed on a Cytoflex flow cytometer (Beckman Coulter) and analyzed using the FlowJo software package. T cells were gated based on size, shape, viability, and HLA-B7 expression. Table 10 and FIG. 1 shows the mean percentage of cells negative for HLA-B7 following editing at the HLA-B locus.

TABLE 10
Mean percentage of T cells HLA-B7 negative
following editing at the HLA-B locus

| Guide ID | Mean % B7− | SD % B7− |
|---|---|---|
| G022020 | 90.55 | 1.767767 |
| G022010 | 74.55 | 0.212132 |
| G022046 | 73.95 | 5.868986 |
| G022043 | 71.65 | 0.777817 |
| G022019 | 60.8 | 2.262742 |
| G022053 | 57.25 | 1.626346 |
| G022011 | 56.4 | 1.131371 |
| G022055 | 52.8 | 0.141421 |
| G022031 | 47.85 | 0.494975 |
| G022027 | 44.55 | 1.06066 |
| G022008 | 39.55 | 3.323402 |
| G022022 | 31.1 | 0.424264 |
| G022015 | 28.65 | 0.919239 |
| G022032 | 23.8 | 7.353911 |
| G022044 | 20.7 | 0.848528 |
| G022039 | 20.65 | 2.899138 |
| G022041 | 17.9 | 1.697056 |
| G022033 | 15.05 | 4.596194 |
| G022017 | 14.8 | 0.848528 |
| G022025 | 13.45 | 1.484924 |
| G022028 | 8.575 | 0.360624 |
| G022050 | 7.73 | 0.028284 |
| G022049 | 7.01 | 0.876812 |
| G022014 | 6.5 | 0.042426 |
| G022030 | 6.37 | 0.579828 |
| G022054 | 6.25 | 0.183848 |
| G022051 | 6.225 | 0.586899 |
| G022018 | 5.83 | 1.06066 |
| G022012 | 5.495 | 0.26163 |
| G022047 | 5.465 | 0.318198 |
| G022048 | 5.44 | 0.028284 |
| G022024 | 4.35 | 1.046518 |
| G022042 | 3.255 | 0.855599 |
| G022034 | 3.07 | 1.499066 |
| G022036 | 2.595 | 0.205061 |
| G022029 | 2.4 | 0.452548 |
| G022026 | 2.08 | 0.692965 |
| G022045 | 1.525 | 0.148492 |
| G022038 | 1.47 | 0.523259 |
| G022035 | 1.34 | 0.39598 |
| G022040 | 1.165 | 0.487904 |
| G022037 | 0.74 | 0.056569 |
| G022021 | 0.64 | 0.155563 |
| G022023 | 0.57 | 0.212132 |
| G022052 | 0.43 | 0.028284 |
| G022013 | 0.41 | 0.028284 |
| G022016 | 0.195 | 0.06364 |
| G022009 | 0.1845 | 0.120915 |

### Example 3: NK Cell Functional Killing Assays

T cells edited to disrupt HLA-B (G022010 and G022020), HLA-A, or B2M were tested for their ability to resist natural killer (NK) cell mediated killing.

### 3.1 Flow Cytometry

NK cell mediated cytotoxicity towards engineered T cells was assayed. For this the T cells were co-cultured with the HLA-B/C matched CTV labelled NK cells at effector to target ratios (E:T) of 10:1, 5:1, 2.5:1, 1.25:1 and 0.625:1 for 21 hours. The cells were stained with 7AAD (BD Pharmingen, Cat. 559925), processed on a Cytoflex flow cytometer (Beckman Coulter) and analyzed using the FlowJo software package. T cells were gated based on CTV negativity, size, and shape and viability. Table 11 and FIG. 2 shows the percentage of T cell lysis following NK cell challenge.

TABLE 11
Percentage T cell lysis following NK cell challenge to engineered T cells

| | WT | | HLA-A KO | | HLA-B KO (010) | | HLA-B KO (020) | | B2M KO | |
|---|---|---|---|---|---|---|---|---|---|---|
| E:T | Mean | SD | Mean | SD | Mean | SD | Mean | SD | Mean | SD |
| Basal | 26.90 | 1.13 | 27.55 | 0.21 | 35.20 | 0.14 | 29.95 | 0.49 | 27.75 | 1.06 |
| 0.625 | 20.70 | 0.00 | 22.30 | 0.71 | 31.50 | 0.42 | 25.50 | 0.14 | 42.60 | 0.71 |
| to 1 | | | | | | | | | | |
| 1.25 to | 20.80 | 0.42 | 23.85 | 0.64 | 32.00 | 1.70 | 25.65 | 0.21 | 56.60 | 1.13 |
| 1 | | | | | | | | | | |
| 2.5 to 1 | 21.10 | 0.14 | 25.75 | 0.35 | 33.00 | 1.27 | 28.30 | 0.28 | 78.25 | 1.34 |
| 5 to 1 | 22.25 | 0.49 | 27.40 | 0.28 | 35.05 | 0.92 | 30.40 | 0.14 | 89.19 | 3.66 |
| 10 to 1 | 22.90 | 0.14 | 27.65 | 1.20 | 36.35 | 1.63 | 47.00 | 17.39 | 94.79 | 0.38 |

### Example 4: Off-Target Analysis of HLA-B Human Guides

Screening for potential off-target genomic sites cleaved by Cas9 targeting HLA-B was performed. (See, e.g., Cameron et al., Nature Methods. 6, 600-606; 2017). In this experiment, 2 sgRNAs targeting human HLA-B and three control guides targeting EMX1, VEGFA, and RAG1B with known off-target profiles were screened using purified genomic DNA from lymphoblast cell line NA24385 (Coriell Institute). Genomic DNA was treated with Quick CIP (NEB M0525) prior to running SITE-Seq. The number of potential off-target sites were detected using a sgRNA as shown in Table 12 at a concentration of 48 nM sgRNA and 16 nM RNP in the biochemical assay. The assay identified potential off-target sites for the sgRNAs tested.

TABLE 12
Off-Target Analysis

| | | | Off- |
|---|---|---|---|
| | | | Target |
| | | Guide Sequence | Site |
| gRNA ID | Target | (SEQ ID NO:) | Count |
| G022010 | HLA-B | AACAAUGCCCACGAUGGGGA | 37 |
| | | (SEQ ID NO: 3) | |
| G022020 | HLA-B | ACAUGCCAUGUACAGCAUGA | 24 |
| | | (SEQ ID NO: 13) | |
| G000644 | EMX1 | GAGUCCGAGCAGAAGAAGAA | 58 |
| | | (SEQ ID NO: 3107) | |
| G000645 | VEGFA | GACCCCCUCCACCCCGCCUC | 793 |
| | | (SEQ ID NO: 3108) | |
| G000646 | RAG1B | GACUUGUUUUCAUUGUUCUC | 47 |
| | | (SEQ ID NO: 3109) | |

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically overrepresents the number of potential off-target sites as the assay utilizes purified high molecular weight genomic DNA free of the cell environment and is dependent on the dose of Cas9 RNP used. Accordingly, potential off-target sites identified by these methods may be validated using targeted sequencing of the identified potential off-target sites.

### Example 5: Re-Screening of HLA-B Guide RNAs with Cas9

91 sgRNAs in either 100-mer or 91-mer format designed for the disruption of the HLA-B gene were screened for efficacy in T cells by assessing loss of HLA-B surface protein. The donors had an HLA-B phenotype of B*07:02/B*08:01. The percentage of T cells negative for HLA-B7 or HLA-B8 was determined by flow cytometry following editing at the HLA-B locus by HTP-LNP delivery and each test guide.

5.1. Cell Activation and Transfection with HTP-LNP

One day post thaw, T cells (StemCell, HLA-B*07:02/B*08:01) were plated at a density of 0.5×10\hat{ }6 cells/mL in TCAM media supplemented with cytokines: 100 U/mL of IL-2 (Peprotech, Cat. 200-02), 5 ng/mL of IL-7, and 5 ng/mL of IL-15. T cells were activated with TransAct™ (1:100 dilution, Miltenyi Biotec). HTP-LNPs of each guide or control guide were added to the respective wells in the 96 well-plate at a concentration of 2.5 ug/mL on the cells. A solution of ApoE3 in TCAM was added to all wells to make the final concentration of ApoE3 2.5 μg/mL, and plates were incubated at 37° C. Cell plates were split every 2-3 days and each replicate plate (as well as the original plate) were supplemented with TCAM supplemented with cytokines.

8-11 days post transfection with LNPs, plates were spun down for 5 minutes at 500×g, media was aspirated, and cells were stained for flow cytometry readout.

### 5.2. Flow Cytometry

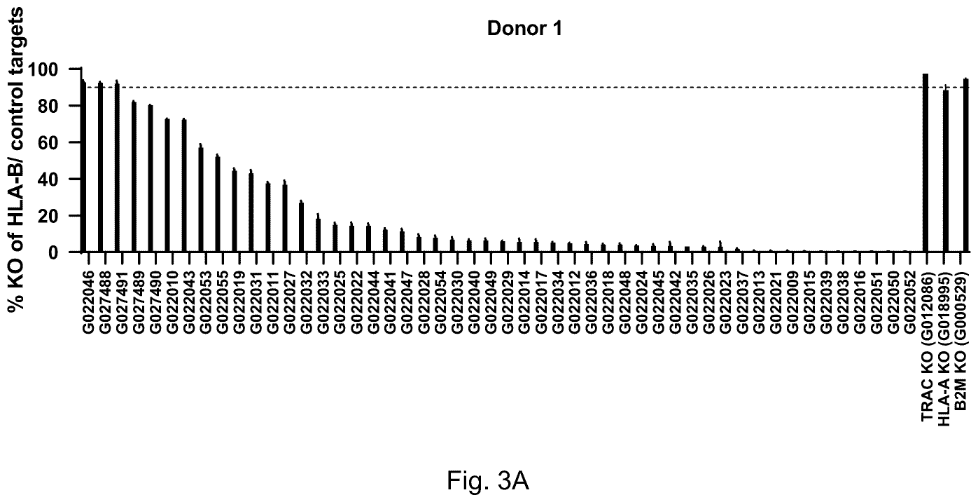

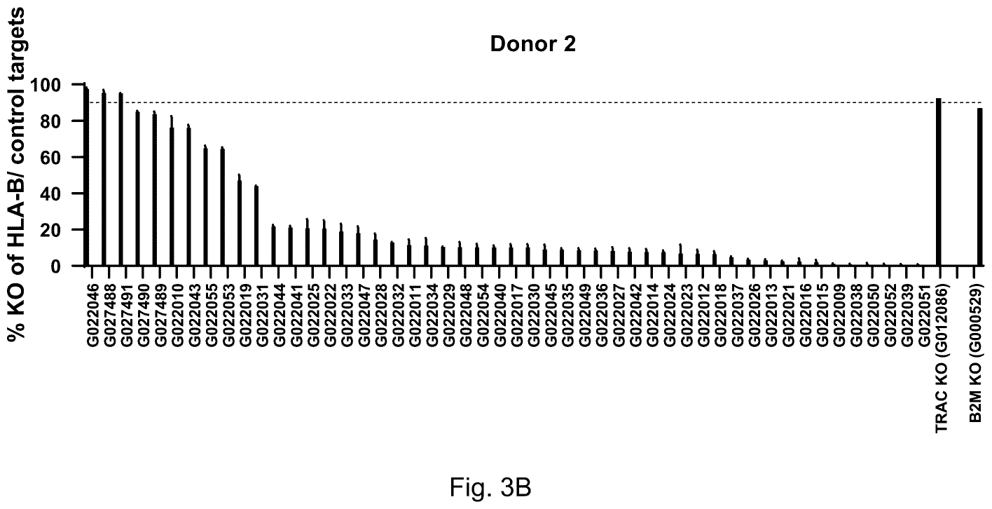

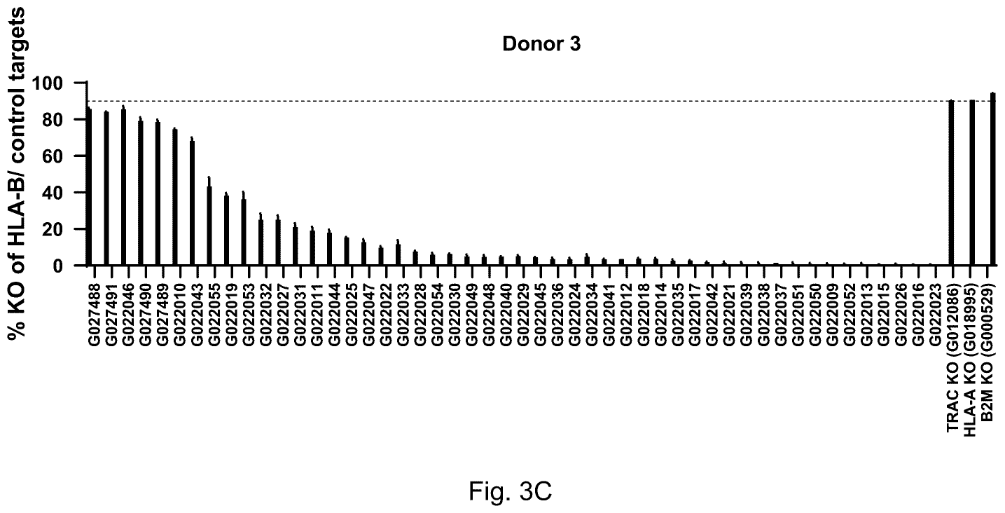

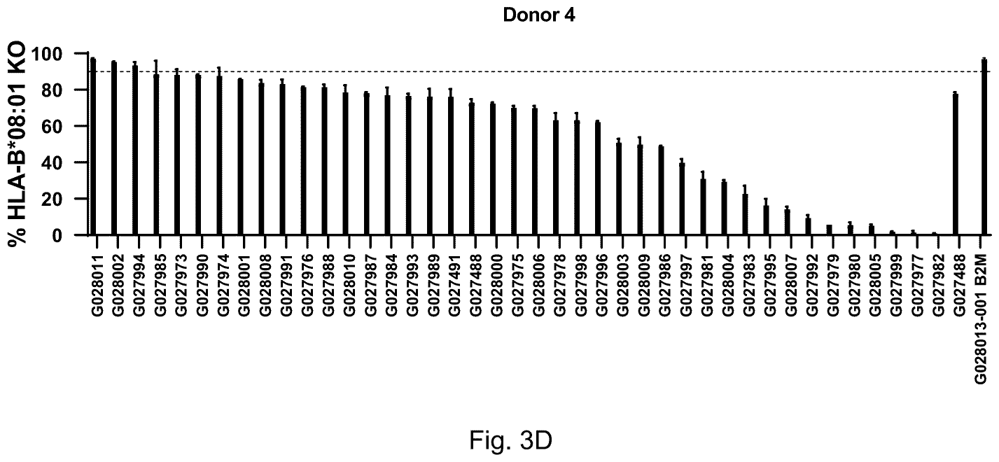

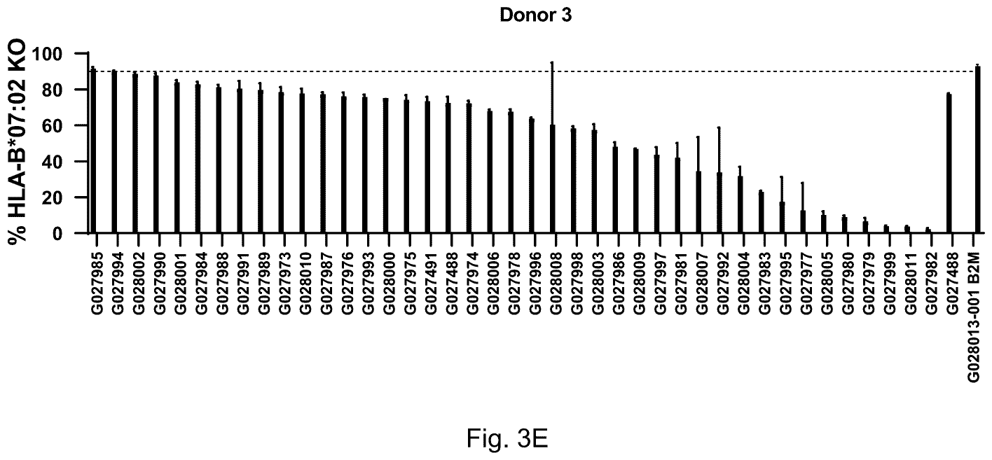

T cells were resuspended in a master mix of 100 μL of FACS buffer containing a 1:100 v/v solution of antibodies targeting HLA-A2 (Invitrogen, Cat. 17-9876-43), HLA-A3 (Invitrogen, Cat. 12-5754-42), HLA-B7 (Miltenyi, Cat. 130-120-234), or HLA-B8 (Miltenyi, Cat. 130-118-366) corresponding to the cells donor's HLA-A or HLA-B (HLA-B*07:02/B*08:01) genotype. Cells were subsequently washed to remove any excess unbound antibodies and run on a Cytoflex flow cytometer (Beckman Coulter) and analyzed using the FlowJo software package. T cells were gated based on size, shape, viability, and HLA-B7, HLA-B8 expression. Tables 13 and 14 and FIGS. 3A-C and 3D-E show the mean percentage of cells negative for HLA-B following editing at the HLA-B locus.

TABLE 13
Mean percentage of HLA-B knockout across 3 donors in 100-mers plus four 91-mers

| | Donor 2 | | Donor 3 | | Donor 1 | |
|---|---|---|---|---|---|---|
| | Mean % | SD % | Mean % | SD % | Mean % | SD % |
| Guide ID | HLA-B KO | HLA-B KO | HLA-B KO | HLA-B KO | HLA-B KO | HLA-B KO |
| G022008 (100mer) | N/A | N/A | N/A | N/A | N/A | N/A |
| G022009 (100mer) | 0.605 | 0.262 | 1.165 | 0.177 | 1.155 | 0.078 |
| G022010 (100mer) | 72.8 | 0.141 | 76.15 | 6.293 | 74.7 | 0.283 |
| G022011 (100mer) | 37.5 | 0.849 | 11.44 | 3.055 | 19.1 | 2.121 |
| G022012 (100mer) | 4.725 | 0.262 | 6.62 | 2.249 | 3.57 | 0.014 |
| G022013 (100mer) | 0.73 | 0.156 | 2.905 | 0.629 | 0.99 | 0.382 |
| G022014 (100mer) | 5.565 | 1.761 | 7.605 | 1.747 | 3.71 | 0.438 |
| G022015 (100mer) | 0.345 | 0.29 | 1.84 | 1.344 | 0.66 | 0.085 |
| G022016 (100mer) | 0.265 | 0.035 | 2.225 | 1.945 | 0.54 | 0.042 |
| G022017 (100mer) | 5.535 | 1.379 | 10.115 | 1.817 | 2.82 | 0.099 |
| G022018 (100mer) | 4.145 | 0.375 | 6.465 | 1.563 | 3.81 | 0.396 |
| G022019 (100mer) | 44.4 | 1.273 | 47.1 | 3.111 | 38.2 | 1.414 |
| G022020 (100mer) | N/A | N/A | N/A | N/A | N/A | N/A |
| G022021 (100mer) | 0.71 | 0.127 | 2.375 | 0.488 | 1.31 | 0.834 |
| G022022 (100mer) | 14.35 | 1.768 | 20.55 | 4.596 | 9.715 | 0.969 |
| G022023 (100mer) | 2.96 | 2.729 | 6.675 | 4.985 | 0.465 | 0.078 |
| G022024 (100mer) | 3.615 | 0.163 | 7.515 | 0.955 | 3.425 | 0.742 |
| G022025 (100mer) | 14.95 | 1.061 | 20.65 | 5.162 | 15.45 | 0.212 |
| G022026 (100mer) | 2.965 | 0.29 | 3.31 | 0.509 | 0.775 | 0.262 |
| G022027 (100mer) | 36.75 | 2.333 | 8.2 | 2.008 | 25.05 | 2.333 |
| G022028 (100mer) | 8.4 | 1.344 | 14.4 | 3.394 | 7.72 | 0.396 |
| G022029 (100mer) | 5.92 | 0.212 | 10.35 | 0.354 | 5.27 | 0.467 |
| G022030 (100mer) | 6.795 | 1.365 | 10.095 | 1.846 | 6.33 | 0.311 |
| G022031 (100mer) | 43.1 | 1.697 | 43.9 | 0.283 | 21.1 | 2.121 |
| G022032 (100mer) | 27 | 0.99 | 12.85 | 0.212 | 25.1 | 3.394 |
| G022033 (100mer) | 18.35 | 2.333 | 18.85 | 4.313 | 11.7 | 2.121 |
| G022034 (100mer) | 5.22 | 0.354 | 11.195 | 4.108 | 4.785 | 1.28 |
| G022035 (100mer) | 3.165 | 0.021 | 8.795 | 0.983 | 2.585 | 0.785 |
| G022036 (100mer) | 4.37 | 0.99 | 8.395 | 1.110 | 3.5 | 0.792 |
| G022037 (100mer) | 1.905 | 0.177 | 4.58 | 0.580 | 1.53 | 0.014 |
| G022038 (100mer) | 0.305 | 0.035 | 1.01 | 0.042 | 1.05 | 0.82 |
| G022039 (100mer) | 0.315 | 0.035 | 0.76 | 0.226 | 1.115 | 0.926 |
| G022040 (100mer) | 6.35 | 0.552 | 10.12 | 1.103 | 4.965 | 0.177 |
| G022041 (100mer) | 12.25 | 0.636 | 21.05 | 0.919 | 3.36 | 0.368 |
| G022042 (100mer) | 3.34 | 2.164 | 7.72 | 2.051 | 1.875 | 0.29 |
| G022043 (100mer) | 72.4 | 0.424 | 76 | 1.697 | 68.25 | 1.909 |
| G022044 (100mer) | 14.3 | 1.273 | 21.65 | 0.919 | 17.95 | 1.768 |
| G022045 (100mer) | 3.355 | 0.969 | 8.945 | 2.765 | 4.575 | 0.163 |
| G022046 (100mer) | 92.75 | 1.061 | 97.4 | 0.849 | 85.5 | 1.838 |
| G022047 (100mer) | 11.25 | 1.485 | 17.95 | 3.748 | 12.85 | 1.485 |
| G022048 (100mer) | 4.13 | 0.622 | 10.31 | 2.814 | 4.765 | 1.025 |
| G022049 (100mer) | 6.26 | 1.188 | 8.495 | 1.280 | 5.01 | 1.004 |
| G022050 (100mer) | 0.195 | 0.092 | 0.985 | 0.643 | 0.995 | 0.417 |
| G022051 (100mer) | 0.255 | 0.092 | 0.67 | 0.141 | 1.03 | 0.721 |
| G022052 (100mer) | 0.155 | 0.049 | 0.78 | 0.382 | 0.845 | 0.276 |
| G022053 (100mer) | 57.05 | 1.909 | 64.4 | 0.990 | 36.3 | 3.96 |
| G022054 (100mer) | 7.855 | 1.096 | 10.18 | 1.867 | 6.04 | 0.849 |
| G022055 (100mer) | 52.05 | 1.202 | 64.75 | 1.485 | 43.25 | 5.02 |
| G027488 (91mer) | 92.55 | 0.354 | 95.3 | 1.697 | 85.65 | 0.778 |
| G027489 (91mer) | 81.9 | 0.566 | 83.45 | 1.626 | 78.65 | 1.202 |
| G027490 (91mer) | 80.35 | 0.071 | 84.75 | 0.636 | 79.2 | 1.838 |
| G027491 (91mer) | 92 | 1.556 | 95.05 | 0.212 | 84.25 | 0.071 |
| G000529 B2M | 94.7 | 0.283 | 86.9 | 0 | 94.4 | 0.424 |
| G028013 B2M | N/A | N/A | N/A | N/A | 92.95 | 0.919 |
| G018995 HLA-A | 88.4 | 2.828 | N/A | N/A | 90.7 | 0 |
| G012086 TRAC | 97.5 | 0 | 92.4 | 0 | 90.4 | 0.283 |

TABLE 14
Mean percentage of HLA-B knockout
across 2 donors in 91-mer format

| | |
|---|---|
| Donor 3 | Donor 4 |

| | Mean % | SD % | Mean % | SD % |
|---|---|---|---|---|
| | HLA-B | HLA-B | HLA-B | HLA-B |
| Guide ID | KO | KO | KO | KO |
| G027488 (91mer) | 72.4 | 3.536 | 72.85 | 1.909 |
| G027488 (91mer) | 77.35 | 0.495 | 77.85 | 0.778 |
| G027491 (91mer) | 73.35 | 2.475 | 76.15 | 4.172 |
| G027973 (91mer) | 78.6 | 2.687 | 88.1 | 3.111 |
| G027974 (91mer) | 72.2 | 1.414 | 87.55 | 4.596 |
| G027975 (91mer) | 74.2 | 2.687 | 69.95 | 1.202 |
| G027976 (91mer) | 76.05 | 2.192 | 81.4 | 0.424 |
| G027977 (91mer) | 12.65 | 15.203 | 1.415 | 0.898 |
| G027978 (91mer) | 67.65 | 1.344 | 63.2 | 3.96 |
| G027979 (91mer) | 6.46 | 2.093 | 5.725 | 0.007 |
| G027980 (91mer) | 8.945 | 0.856 | 5.51 | 1.471 |
| G027981 (91mer) | 41.9 | 8.202 | 30.95 | 3.889 |
| G027982 (91mer) | 2.28 | 0.566 | 0.875 | 0.134 |
| G027983 (91mer) | 23.05 | 0.495 | 22.6 | 4.525 |
| G027984 (91mer) | 82.85 | 1.485 | 76.95 | 4.313 |
| G027985 (91mer) | 91.5 | 0.99 | 88.45 | 7.566 |
| G027986 (91mer) | 48.2 | 2.404 | 48.9 | 0.283 |
| G027987 (91mer) | 77.3 | 1.131 | 78.05 | 0.636 |
| G027988 (91mer) | 81.2 | 1.414 | 81.3 | 1.556 |
| G027989 (91mer) | 79.65 | 3.748 | 76.3 | 4.243 |
| G027990 (91mer) | 87.75 | 1.061 | 88.1 | 0.283 |
| G027991 (91mer) | 80.45 | 4.313 | 83 | 2.546 |
| G027992 (91mer) | 33.75 | 24.961 | 9.25 | 1.768 |
| G027993 (91mer) | 75.85 | 1.202 | 76.5 | 1.273 |
| G027994 (91mer) | 90.05 | 0.354 | 93.45 | 1.768 |
| G027995 (91mer) | 17.4 | 14.001 | 16.3 | 3.677 |
| G027996 (91mer) | 63.75 | 0.636 | 62.05 | 0.778 |
| G027997 (91mer) | 43.7 | 4.101 | 39.8 | 2.121 |
| G027998 (91mer) | 58.35 | 1.061 | 63.2 | 3.96 |
| G027999 (91mer) | 3.78 | 0.424 | 1.77 | 0.24 |
| G028000 (91mer) | 75.3 | 0 | 72.2 | 0.849 |
| G028001 (91mer) | 83.85 | 1.344 | 85.8 | 0.141 |
| G028002 (91mer) | 88.6 | 0.99 | 95.3 | 0.283 |
| G028003 (91mer) | 57.45 | 3.182 | 50.9 | 2.121 |
| G028004 (91mer) | 31.75 | 5.162 | 29.25 | 1.061 |
| G028005 (91mer) | 10.13 | 1.937 | 5.19 | 0.75 |
| G028006 (91mer) | 67.95 | 0.778 | 69.8 | 1.273 |
| G028007 (91mer) | 34.5 | 18.95 | 14.1 | 1.556 |
| G028008 (91mer) | 60.35 | 34.578 | 83.75 | 1.768 |
| G028009 (91mer) | 46.9 | 0.283 | 49.9 | 3.96 |
| G028010 (91mer) | 77.7 | 2.687 | 78.5 | 3.96 |
| G028011 (91mer) | 3.675 | 0.247 | 97.15 | 0.212 |
| G000529 B2M | 94.4 | 0.424 | N/A | N/A |
| G028013 B2M | 92.95 | 0.919 | 96.85 | 0.636 |
| G018995 HLA-A | 90.7 | 0 | N/A | N/A |
| G012086 TRAC | 90.4 | 0.283 | N/A | N/A |

### Example 6: LNP Dose Response Curves for Top HLA-B Spy Cleavase Guides

### 6.1 T Cell Preparation

One day post thaw, T cells (Cellex, HLA-B*07:02/B*08:01) were plated at a density of 0.5×10\hat{ }6 cells/mL in TCAM media supplemented with 2× cytokines: 200 U/mL of IL-2 (Peprotech, Cat. 200-02), 10 ng/mL of IL-7, and 10 ng/mL of IL-15. T cells were activated with TransAct™ (1:100 dilution, Miltenyi Biotec). TCAM Media containing 4× ApoE3 (10 ug/mL) was prepared and filled in a reservoir for the Hamilton. To “Column 1” of a Hamilton compatible deep well dilution block, a 1 mL 4× stock solution (20 μg/mL) of each HLA-B LNP in TCAM (for guides G027488, G027489, G027490, G027491) was added in duplicates, according to the plate layout. Transfection was performed on Hamilton by diluting the LNP stock and ApoE3 media 4-fold to generate the highest point in the standard curve (5 ug/mL LNP), followed by a 2-fold serial dilution to obtain a 12-point DRC. The ApoE3 concentration was kept constant at 2.5 μg/mL in each well. The total volume of LNP+ ApoE3 media added to each well by the Hamilton to get the desired concentrations was 100 μL. Transfection of the B2M LNP was performed manually without the Hamilton, by adding B2M LNP to the cells at a concentration of 2.5 μg/mL and ApoE3 at a concentration of 2.5 ug/mL in TCAM. It is to be noted that since cells were plated with 2× cytokines, LNP and ApoE3 containing media was cytokine free to result in a 1× final concentration of cytokines on the cells (100 U/mL of IL-2 (Peprotech, Cat. 200-02), 5 ng/mL of IL-7, and 5 ng/mL of IL-15). Plates were transferred to at 37° C. incubator. Cell plates were split every 2-3 days and each replicate plate (as well as the original plate) were supplemented with TCAM supplemented with cytokines.
9 days post transfection with LNPs, plates were spun down for 5 minutes at 500×g, media was aspirated, and cells were stained for flow cytometry readout.

### 6.2 Flow Cytometry

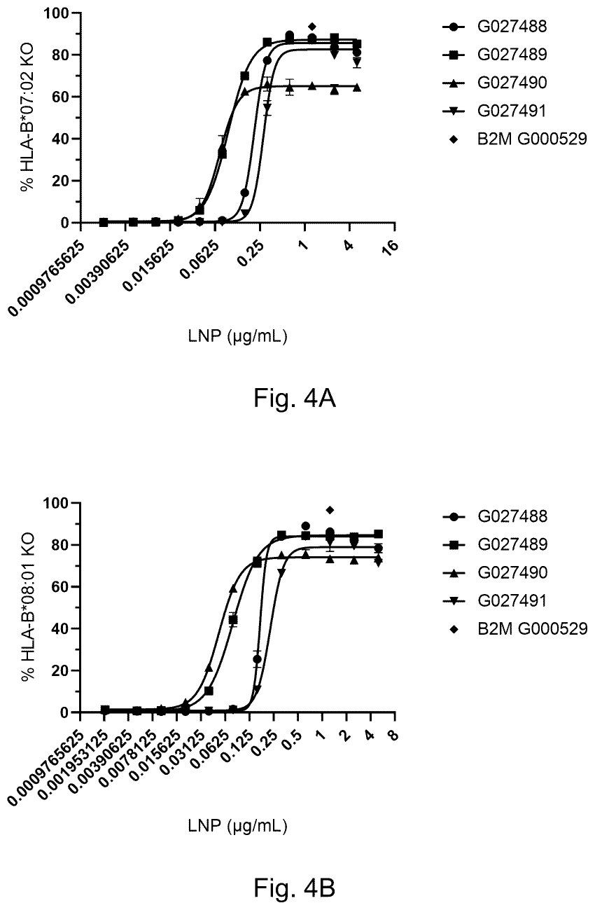

Flow cytometry was performed as in Example 5.2. Tables 15 and 16 and FIGS. 4A and 4B show the percent knockout at each LNP dose.

TABLE 15

| | G027488 | | | G027489 | | | G027490 | | |
|---|---|---|---|---|---|---|---|---|---|
| | Mean % | SD % | | Mean % | SD % | | Mean % | SD % | |
| Guide | HLA- | HLA- | | HLA- | HLA- | | HLA- | HLA- | |
| sgRNA | B*07:02 | B*07:02 | | B*07:02 | B*07:02 | | B*07:02 | B*07:02 | |
| (μg/mL) | KO | KO | N | KO | KO | N | KO | KO | N |
| 5 | 81.20 | 1.27 | 2 | 85.25 | 1.63 | 2 | 64.65 | 1.06 | 2 |
| 2.5 | 83.80 | 2.12 | 2 | 88.30 | 1.41 | 2 | 63.50 | 2.40 | 2 |
| 1.25 | 88.25 | 0.21 | 2 | 87.20 | 1.70 | 2 | 65.35 | 1.48 | 2 |
| 0.625 | 89.65 | 0.92 | 2 | 87.05 | 0.49 | 2 | 64.65 | 3.89 | 2 |
| 0.313 | 77.35 | 0.64 | 2 | 86.15 | 0.35 | 2 | 66.15 | 3.32 | 2 |
| 0.156 | 14.25 | 0.78 | 2 | 70.05 | 1.91 | 2 | 62.70 | 1.41 | 2 |
| 0.078 | 1.13 | 0.37 | 2 | 32.60 | 0.42 | 2 | 36.60 | 4.95 | 2 |
| 0.039 | 0.53 | 0.44 | 2 | 5.92 | 0.22 | 2 | 8.01 | 3.52 | 2 |
| 0.02 | 0.22 | 0.21 | 2 | 1.02 | 0.01 | 2 | 1.90 | 0.07 | 2 |
| 0.01 | 0.62 | 0.16 | 2 | 0.33 | 0.04 | 2 | 0.40 | 0.11 | 2 |
| 0.005 | 0.38 | 0.06 | 2 | 0.31 | 0.00 | 2 | 0.29 | 0.18 | 2 |
| 0.002 | 0.28 | 0.10 | 2 | 0.12 | 0.00 | 2 | 0.13 | 0.01 | 2 |

| | G027491 | | | G000529 B2M | | |
|---|---|---|---|---|---|---|
| | Mean % | SD % | | Mean % | SD % | |
| Guide | HLA- | HLA- | | HLA- | HLA- | |
| sgRNA | B*07:02 | B*07:02 | | B*07:02 | B*07:02 | |
| (μg/mL) | KO | KO | N | KO | KO | N |
| 5 | 76.25 | 2.47 | 2 | | | |
| 2.5 | 79.80 | 0.14 | 2 | | | |
| 1.25 | 87.00 | 0.71 | 2 | 93.5 | | 1 |
| 0.625 | 86.95 | 0.92 | 2 | | | |
| 0.313 | 54.70 | 3.54 | 2 | | | |
| 0.156 | 4.28 | 0.53 | 2 | | | |
| 0.078 | 0.33 | 0.01 | 2 | | | |
| 0.039 | 0.14 | 0.01 | 2 | | | |
| 0.02 | 0.13 | 0.00 | 2 | | | |
| 0.01 | 0.13 | 0.03 | 2 | | | |
| 0.005 | 0.10 | 0.00 | 2 | | | |
| 0.002 | 0.22 | 0.11 | 2 | | | |

TABLE 16

| | G027488 | | | G027489 | | | G027490 | | |
|---|---|---|---|---|---|---|---|---|---|
| | Mean % | SD % | | Mean % | SD % | | Mean % | SD % | |
| Guide | HLA- | HLA- | | HLA- | HLA- | | HLA- | HLA- | |
| sgRNA | B*08:01 | B*08:01 | | B*08:01 | B*08:01 | | B*08:01 | B*08:01 | |
| (μg/mL) | KO | KO | N | KO | KO | N | KO | KO | N |
| 5 | 78.45 | 2.05 | 2 | 85.20 | 0.14 | 2 | 74.05 | 2.19 | 2 |
| 2.5 | 82.20 | 1.27 | 2 | 83.80 | 0.14 | 2 | 72.75 | 0.49 | 2 |
| 1.25 | 86.25 | 0.07 | 2 | 84.10 | 0.28 | 2 | 73.35 | 1.91 | 2 |
| 0.625 | 89.00 | 0.71 | 2 | 84.45 | 0.78 | 2 | 75.60 | 2.12 | 2 |
| 0.313 | 84.05 | 1.63 | 2 | 84.75 | 0.92 | 2 | 75.20 | 1.27 | 2 |
| 0.156 | 25.45 | 3.89 | 2 | 71.80 | 2.40 | 2 | 71.60 | 1.41 | 2 |
| 0.078 | 1.64 | 0.35 | 2 | 44.30 | 3.39 | 2 | 59.25 | 1.91 | 2 |
| 0.039 | 0.61 | 0.22 | 2 | 10.30 | 1.00 | 2 | 21.55 | 0.21 | 2 |
| 0.02 | 0.47 | 0.18 | 2 | 1.88 | 0.23 | 2 | 4.84 | 0.59 | 2 |
| 0.01 | 0.48 | 0.20 | 2 | 0.90 | 0.02 | 2 | 1.98 | 0.35 | 2 |
| 0.005 | 0.61 | 0.11 | 2 | 0.81 | 0.04 | 2 | 1.02 | 0.22 | 2 |
| 0.002 | 0.76 | 0.11 | 2 | 1.37 | 0.65 | 2 | 1.36 | 0.76 | 2 |

| | G027491 | | | G000529 B2M | | |
|---|---|---|---|---|---|---|
| | Mean % | SD % | | Mean % | SD % | |
| Guide | HLA- | HLA- | | HLA- | HLA- | |
| sgRNA | B*08:01 | B*08:01 | | B*08:01 | B*08:01 | |
| (μg/mL) | KO | KO | N | KO | KO | N |
| 5 | 71.25 | 0.49 | 2 | | | |
| 2.5 | 79.45 | 0.21 | 2 | | | |
| 1.25 | 81.10 | 4.24 | 2 | 96.6 | | 1 |
| 0.625 | 84.00 | 1.84 | 2 | | | |
| 0.313 | 66.45 | 1.06 | 2 | | | |
| 0.156 | 10.75 | 0.64 | 2 | | | |
| 0.078 | 1.24 | 0.27 | 2 | | | |
| 0.039 | 0.73 | 0.08 | 2 | | | |
| 0.02 | 1.09 | 0.52 | 2 | | | |
| 0.01 | 0.95 | 0.23 | 2 | | | |
| 0.005 | 0.78 | 0.25 | 2 | | | |
| 0.002 | 0.72 | 0.13 | 2 | | | |

### Example 7: Screening of HLA-B Guides with Nme2 BC22n and Nme2 Cleavase

57 sgRNAs targeting HLA-B were screened at a fixed concentration of 100 μM. sgRNAs targeting TRAC and B2M were used as controls. Guides were either electroporated with mRNA encoding UGI (3490 ng/mL) (SEQ ID NO: 821) as well as either mRNA encoding Nme2 BC22n (1709 ng/mL) (SEQ ID NO: 822), an mRNA encoding Nme2-cleavase (1660 ng/mL) (SEQ ID NO: 825), or an mRNA encoding Spy-cleavase (2230 ng/mL) (SEQ ID NO: 827).

7.1 T cell Preparation

Healthy human donor apheresis was obtained commercially (Hemacare), and cells were washed and resuspended in in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930). Upon thaw, T cells were plated at a density of 1.0×10⁶ cells/mL in T cell growth media (TCGM) composed of CTS OpTmizer T Cell Expansion SFM and T Cell Expansion Supplement (ThermoFisher Cat. A1048501) containing 5% human AB serum (GeminiBio, Cat. 100-512), 1× Penicillin-Streptomycin, 1× Glutamax, 10 mM HEPES, 1× cytokines (200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15)). T cells were rested in the T cell growth media for 24 hours at which time they were activated with TransAct™ (1:100 dilution, Miltenyi Biotec, Cat. 130-111-160). T cells were activated 48 hours prior to electroporation.

7.2 T Cell Editing with RNA Electroporation

T cells were edited at the HLA-B locus with Cas9 (SEQ ID NO: 827), mRNA encoding Nine BC22n (SEQ ID NO: 822) and UGI (SEQ ID NO: 821) to assess sgRNA editing efficacy and the corresponding loss of HLA-B7 expression.

A solution containing mRNA encoding BC22n (SEQ ID: 822) and UGI (SEQ ID NO: 821) was prepared in P3 electroporation buffer (Lonza Catalog #V4SP-3960). 100 μM HLA-B targeting sgRNAs included in Table 17 were removed from their storage plates and denatured for 2 minutes at 95° C. and incubated at room temperature for 5 minutes. Forty-eight hours post activation, T cells were harvested, centrifuged at 500 g for 5 minutes, and resuspended at a concentration of 12.5×10⁶ T cells/mL in P3 electroporation buffer (Lonza Catalog #V4SP-3960). For each well to be electroporated, 1×10⁵ T cells were mixed with 20 ng/μL of BC22n mRNAs, 20 ng/μL of UGI mRNA and 20 pmols of sgRNA in a final volume of 20 μL of P3 electroporation buffer. This mix was transferred in duplicate to a 96-well Nucleofector™ plate (manufacturer, catalog #) and electroporated using manufacturer's pulse code. Electroporated T cells were immediately rested in 80 μL of CTS Optimizer T cell growth media (manufacturer, catalog #) without cytokines for 15 minutes. After resting, T cells were transferred to flat-bottom 96-well plates (manufacturer, catalog #) containing 80 μL of CTS Optimizer T cell growth media (manufacturer, catalog #) supplemented with 2× cytokines (200 U/mL recombinant human interleukin-2 (Peprotech, Cat. 200-02), 5 ng/mL recombinant human interleukin-7 (Peprotech, Cat. 200-07), and 5 ng/mL recombinant human interleukin-15 (Peprotech, Cat. 200-15) per well. The plates were incubated at 37° C. for 10 days.

On day 7 post-electroporation, cells were collected for flow cytometry analysis. NGS analysis of cells was run by a third-party facility using standard methods.

### 7.3 Flow Cytometry

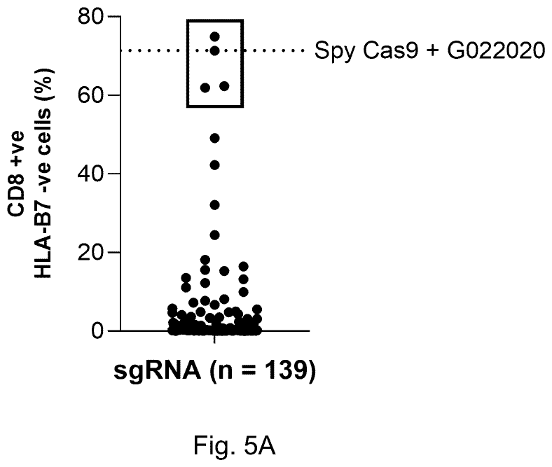

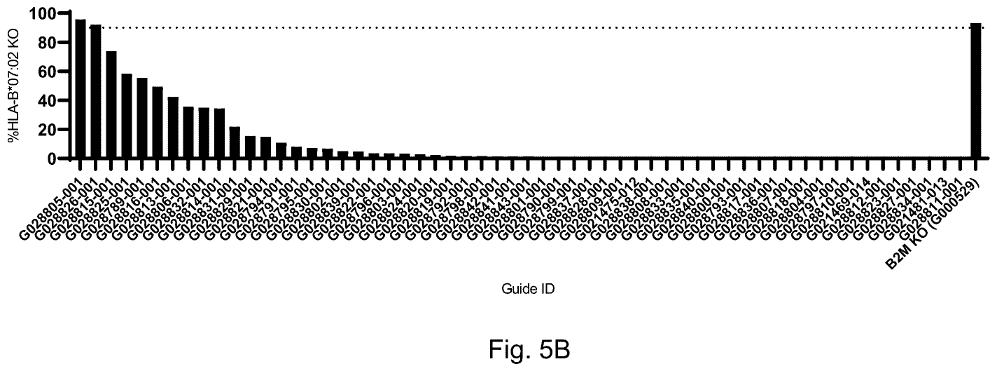

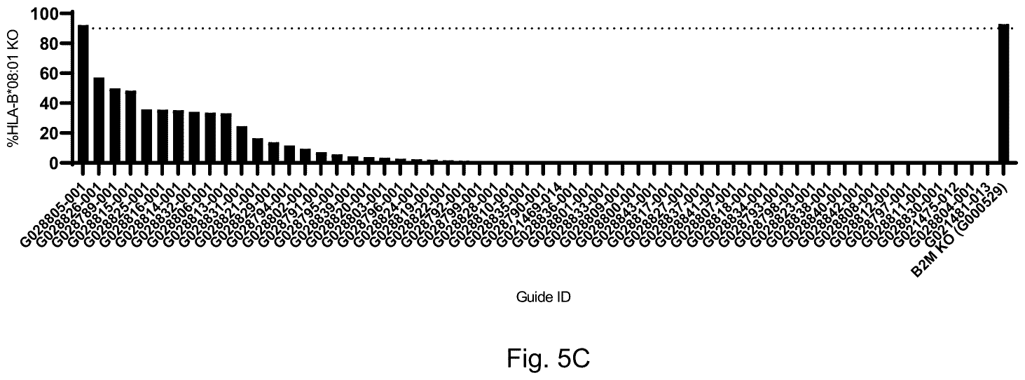

T cells were phenotyped by flow cytometry to determine HLA-B7 protein expression. Briefly, T cells were incubated for 30 min at 4° C. with a mixture of antibodies against CD3 (BioLegend, Cat. No. 316314), CD4 (BioLegend, Cat. No. 317434), CD8 (BioLegend, Cat. No. 301046), Viakrome (Immunotech, Cat. No. C36628), HLA B7 (Milteny Biotech, Cat. No. 130-120-234) diluted at 1:200 in cell staining buffer. Cells were subsequently washed and resuspended in 100 μL of cell staining buffer. Cells were then processed on a Cytoflex flow cytometer (Beckman Coulter) and analyzed using the FlowJo software package. T cells were gated based on size, shape, viability, CD8, CD3, and HLA-B7 expression. Table 17 and FIG. 5A shows the mean percentage of cells negative for HLA-B7 following editing at the HLA-B locus.

TABLE 17
Mean % HLA-B7 T cells following editing at the HLA-B locus.

| | % HLA-B7-ve | | | Guide | % HLA-B7-ve | | |
|---|---|---|---|---|---|---|---|
| Guide ID | Mean | SD | N | ID | Mean | SD | N |
| G018995 | 0.27 | 0.170 | 2 | G028828 | 2.13 | 0.834 | 2 |
| HLA-A | | | | | | | |
| G021469 | 0.08 | 0.000 | 2 | G028829 | 3.375 | 0.940 | 2 |
| TRAC | | | | | | | |
| G021481 | 1.38 | 1.796 | 2 | G028830 | 0.32 | 0.297 | 2 |
| TRAC | | | | | | | |
| G028789 | 49.05 | 1.202 | 2 | G028831 | 32.05 | 0.636 | 2 |
| G028790 | 1.295 | 0.672 | 2 | G028832 | 74.9 | 9.334 | 2 |
| G028791 | 11.075 | 1.591 | 2 | G028833 | 7.215 | 5.353 | 2 |
| G028792 | 0.36 | 0.085 | 2 | G028834 | 0.27 | 0.198 | 2 |
| G028793 | 0.31 | 0.297 | 2 | G028835 | 0.185 | 0.078 | 2 |
| G028794 | 4.295 | 0.530 | 2 | G028836 | 0.415 | 0.120 | 2 |
| G028795 | 71.3 | 6.505 | 2 | G028837 | 0.29 | 0.099 | 2 |
| G028796 | 16.425 | 10.713 | 2 | G028838 | 0.45 | 0.042 | 2 |
| G028797 | 0.51 | 0.113 | 2 | G028839 | 6.69 | 0.990 | 2 |
| G028798 | 0.08 | 0.057 | 2 | G028840 | 2.1 | 0.594 | 2 |
| G028799 | 1.795 | 0.035 | 2 | G028841 | 0.135 | 0.106 | 2 |
| G028800 | 0.12 | 0.071 | 2 | G028842 | 0.06 | 0.042 | 2 |
| G028801 | 0.335 | 0.148 | 2 | G028843 | 0.185 | 0.035 | 2 |
| G028802 | 24.45 | 2.899 | 2 | G028931 | 0.065 | 0.035 | 2 |
| G028803 | 42.25 | 3.465 | 2 | G028932 | 0.12 | 0.028 | 2 |
| G028804 | 7.695 | 2.397 | 2 | | | | |
| G028805 | 9.915 | 0.686 | 2 | | | | |
| G028806 | 18.15 | 0.495 | 2 | | | | |
| G028807 | 4.615 | 0.742 | 2 | | | | |
| G028808 | 0.37 | 0.240 | 2 | | | | |
| G028809 | 0.03 | 0.000 | 2 | | | | |
| G028810 | 0.055 | 0.035 | 2 | | | | |
| G028811 | 0.24 | 0.156 | 2 | | | | |
| G028812 | 0.035 | 0.035 | 2 | | | | |
| G028813 | 1.625 | 0.134 | 2 | | | | |
| G028814 | 3.495 | 0.672 | 2 | | | | |
| G028815 | 12.2 | 0.849 | 2 | | | | |
| G028816 | 13.5 | 0.424 | 2 | | | | |
| G028817 | 1.52 | 0.778 | 2 | | | | |
| G028818 | 0.92 | 1.273 | 2 | | | | |
| G028819 | 0.1 | 0.014 | 2 | | | | |
| G028820 | 0.365 | 0.346 | 2 | | | | |
| G028821 | 0.085 | 0.007 | 2 | | | | |
| G028822 | 0.185 | 0.035 | 2 | | | | |
| G028823 | 1.49 | 0.071 | 2 | | | | |
| G028824 | 0.215 | 0.247 | 2 | | | | |
| G028825 | 1.585 | 0.205 | 2 | | | | |
| G028826 | 0.825 | 0.035 | 2 | | | | |
| G028827 | 0.605 | 0.375 | 2 | | | | |

### 7.4 Nme2 Cleavase Screen

55 sgRNAs targeting HLA-B were screened at a fixed concentration of 100 μM. Three sgRNAs targeting TRAC and a sgRNA targeting B2M were used as controls. The donors had an HLA-B phenotype of B*07:02/B*08:01. The percentage of T cells negative for HLA-B7 or HLA-B8 was determined by flow cytometry following editing at the HLA-B locus by HTP-LNP delivery and each test guide.

T cells were transfected with HTP-LNP and flow cytometry was performed as in Example 5. Table 18 and FIGS. 5B and 5C show the mean percentage of knockout for HLA*B07:02 or HLAB*08:01.

TABLE 18
Mean percentage HLA-B*07:02 or HLA-B*08:01 knockout
following editing at the HLA-B locus

| | % HLA-B*07:02 KO | % HLA-B*08:01 KO |
|---|---|---|
| Guide ID | Mean | Mean |
| G000529 B2M | 93.25 | 92.95 |
| G028789 | 55.5 | 49.8 |
| G028790 | 1.08 | 0.68 |
| G028791 | 8.05 | 7.22 |
| G028792 | 1.73 | 1.39 |
| G028793 | 0.65 | 0.26 |
| G028794 | 10.8 | 11.6 |
| G028795 | 7.24 | 5.67 |
| G028796 | 3.45 | 2.88 |
| G028797 | 0.43 | 0.16 |
| G028798 | 1.69 | 0.22 |
| G028799 | 1.05 | 1.05 |
| G028800 | 0.67 | 0.44 |
| G028801 | 1.21 | 0.48 |
| G028802 | 5.09 | 9.4 |
| G028803 | 3.36 | 3.46 |
| G028804 | 0.44 | 0.13 |
| G028805 | 95.6 | 92.2 |
| G028806 | 35.7 | 33.6 |
| G028807 | 0.49 | 0.34 |
| G028808 | 0.79 | 0.17 |
| G028809 | 0.88 | 0.47 |
| G028810 | 0.42 | 0.83 |
| G028811 | 0.14 | 0.15 |
| G028812 | 0.38 | 0.17 |
| G028813 | 42.4 | 33.2 |
| G028814 | 34.3 | 35.3 |
| G028815 | 73.7 | 48.4 |
| G028816 | 49.4 | 35.7 |
| G028817 | 0.64 | 0.39 |
| G028818 | 0.46 | 0.32 |
| G028819 | 1.81 | 2.14 |
| G028820 | 2.45 | 3.88 |
| G028821 | 14.9 | 16.5 |
| G028822 | 3.47 | 1.71 |
| G028823 | 0.31 | 0.22 |
| G028824 | 2.77 | 2.47 |
| G028825 | 58.3 | 35.8 |
| G028826 | 92.1 | 57.2 |
| G028827 | 0.28 | 0.37 |
| G028828 | 0.89 | 1.01 |
| G028829 | 15.5 | 13.9 |
| G028830 | 6.77 | 0.15 |
| G028831 | 21.9 | 24.6 |
| G028832 | 34.9 | 34.1 |
| G028833 | 0.76 | 0.48 |
| G028834 | 0.21 | 0.3 |
| G028835 | 0.75 | 0.72 |
| G028836 | 0.5 | 0.49 |
| G028837 | 1.03 | 0.36 |
| G028838 | 0.82 | 0.21 |
| G028839 | 4.75 | 4.34 |
| G028840 | 0.68 | 0.21 |
| G028841 | 1.28 | 0.36 |
| G028842 | 1.31 | 0.19 |
| G028843 | 1.25 | 0.43 |
| G021469 TRAC | 0.42 | 0.63 |
| G021475 TRAC | 0.83 | 0.14 |
| G021481 TRAC | 0.19 | 0.12 |

### Example 8: LNP Dose Response Curves (DRC) for Top HLA-A and HLA-B Nme2 Guides

A DRC was run for lead HLA-A and HLA-B sgRNAs along with Nme2 BC22 to determine the best guide for knocking out HLA genes. sgRNAs were titrated in 8-point DRC along with fixed concentration of an mRNA encoding UGI (SEQ ID NO: 821) (3490 ng/L) and an mRNA encoding Nme2 BC22n base editor (SEQ ID NO: 822) (1709 ng/L) or an mRNA encoding a Spy-cleavase (SEQ ID NO: 827) (2230 ng/L) in T cells using electroporation. T cells were then analyzed by flow cytometry to determine editing efficiencies. T cells were prepared as described in Example 1.

8.1 mRNA Electroporation

Solutions containing mRNA encoding BC22n (SEQ ID NO: 822) and UGI (SEQ ID NO: 821) were prepared in P3 buffer. 100 μM HLA-B targeting sgRNAs were removed from their storage plates and denatured for 2 minutes at 95° C. and incubated at room temperature for 5 minutes. Forty-eight hours post activation, T cells were harvested, centrifuged, and resuspended at a concentration of 12.5×10\hat{ }₆ T cells/mL in P3 electroporation buffer (Lonza Catalog #V4SP-3960). Each sgRNA was serially diluted in ratio of 1:2 in P3 electroporation buffer starting from 5 μM in a 96 well PCR plate in duplicate as described in Table 19. Following dilution, 1×10\hat{ }5 T cells, 20 ng/μL of BC22n mRNAs and 20 ng/μL of UGI mRNA were mixed with sgRNA plate to make the final volume of 20 μL of P3 electroporation buffer. The mix was transferred to two 96-well Nucleofector™ plates. Cells were electroporated in duplicate using Lonza shuttle 96w using manufacturer's pulse code. Immediately post electroporation, cells were recovered in 80 μL of TCGM without cytokines at 37° C. for 15 minutes. Electroporated T cells were subsequently cultured in TCGM further supplemented with 2× cytokines (200 U/mL recombinant human interleukin-2 (Peprotech, Cat. 200-02), 5 ng/mL recombinant human interleukin-7 (Peprotech, Cat. 200-07), and 5 ng/mL recombinant human interleukin-15 (Peprotech, Cat. 200-15) per well. The plates were incubated at 37° C. for 10 days. On day 7 post-edit, edited T cells were collected for flow cytometry analysis.

### 8.2 Flow Cytometry

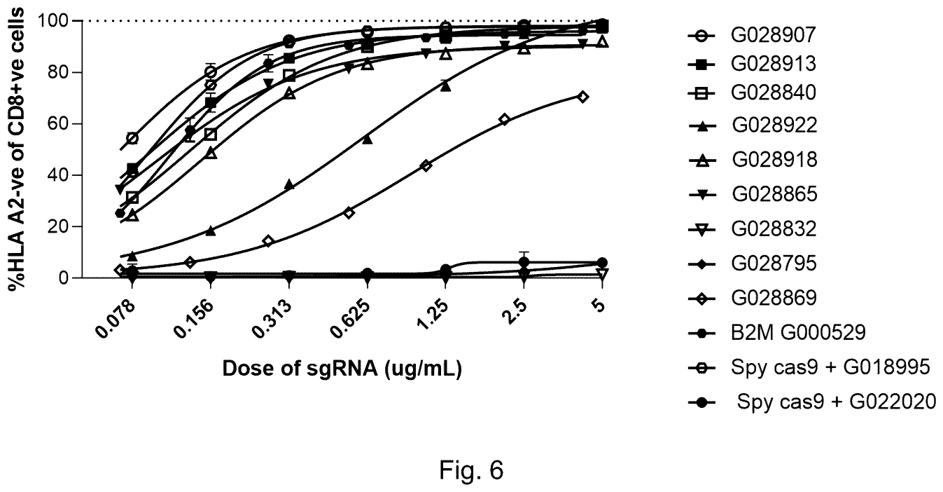

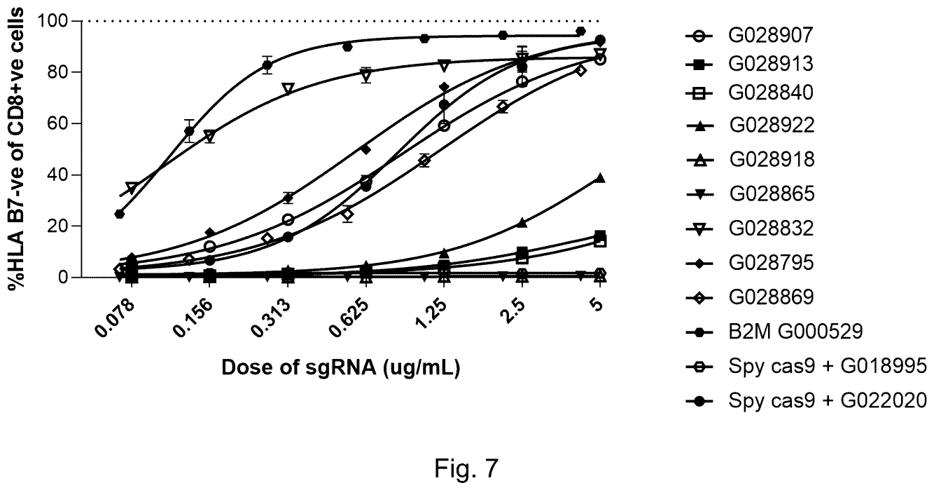

On day 10, cells were transferred to U bottom plates, spun and resuspended in master mix containing antibodies for PerCP/Cy5.5 CD3 (BioLegend, Cat. 317434), BV421 CD4 (BioLegend, Cat. 317434), BV785 CD8 (BioLegend, Cat. 301046), HLA A2 (BioLegend Inc., Cat. 343306), HLA B7 (Miltenyi Biotec Inc., Cat. 130-120-234) at a 1:200 dilution and Viakrome (Immunotech, Cat. C36628) at 1:100 final concentration in FACs buffer and then incubated at 4° C. for 30 minutes. After the incubation, the cells were washed and resuspended in 100 μL FACs buffer (PBS+2% FBS+2 mM EDTA) and processed by flow cytometry using a Beckman Coulter CytoflexS, and analyzed using the FlowJo software package. Tables 19 and 20 and FIGS. 6 and 7 show the percent editing at each sgRNA dose.

TABLE 19
Dose response curve for the percent of HLA-A2⁻ of CD8⁺ cells with
various doses of sgRNA

| | G028907 | | | G028913 | | | G028840 | | |
|---|---|---|---|---|---|---|---|---|---|
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 98.65 | 0.07 | 2 | 97.20 | 0.42 | 2 | 98.00 | 0.14 | 2 |
| 2.5 | 98.25 | 0.21 | 2 | 96.15 | 0.07 | 2 | 97.30 | 0.28 | 2 |
| 1.25 | 97.40 | 0.14 | 2 | 93.30 | 0.00 | 2 | 94.35 | 0.92 | 2 |
| 0.625 | 95.70 | 0.42 | 2 | 90.85 | 0.92 | 2 | 90.00 | 0.99 | 2 |
| 0.313 | 92.40 | 0.00 | 2 | 85.55 | 1.77 | 2 | 78.85 | 0.92 | 2 |
| 0.078 | 80.20 | 3.25 | 2 | 68.25 | 3.75 | 2 | 55.95 | 0.35 | 2 |
| 0 | 54.45 | 2.05 | 2 | 42.70 | 1.13 | 2 | 31.45 | 0.64 | 2 |
| EC50 | 0.07038 | | | 0.08998 | | | 0.1289 | | |
| | G028922 | | | G028918 | | | G028865 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 98.65 | 0.21 | 2 | 92.15 | 0.07 | 2 | 90.95 | 1.06 | 2 |
| 2.5 | 95.00 | 0.99 | 2 | 89.45 | 1.20 | 2 | 90.20 | 1.27 | 2 |
| 1.25 | 74.90 | 2.12 | 2 | 87.25 | 1.06 | 2 | 87.20 | 0.42 | 2 |
| 0.625 | 54.20 | 0.42 | 2 | 83.40 | 0.71 | 2 | 81.35 | 1.34 | 2 |
| 0.313 | 36.80 | 0.85 | 2 | 72.05 | 0.64 | 2 | 75.45 | 1.06 | 2 |
| 0.078 | 18.60 | 0.42 | 2 | 48.80 | 0.85 | 2 | 55.60 | 2.26 | 2 |
| 0 | 8.73 | 0.02 | 2 | 24.65 | 1.20 | 2 | 34.30 | 1.56 | 2 |
| EC50 | 0.6028 | | | 0.1429 | | | 0.09631 | | |
| | G028832 | | | G028795 | | | G028869 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 1.45 | 0.13 | 2 | 5.74 | 0.32 | 2 | 70.55 | 0.49 | 2 |
| 2.5 | 0.71 | 0.03 | 2 | 2.99 | 0.76 | 2 | 61.75 | 0.92 | 2 |
| 1.25 | 0.37 | 0.04 | 2 | 1.65 | 0.10 | 2 | 43.75 | 1.91 | 2 |
| 0.625 | 0.33 | 0.04 | 2 | 0.96 | 0.03 | 2 | 25.45 | 0.92 | 2 |
| 0.313 | 0.49 | 0.15 | 2 | 0.82 | 0.08 | 2 | 14.55 | 0.92 | 2 |
| 0.078 | 0.29 | 0.08 | 2 | 0.39 | 0.01 | 2 | 6.20 | 0.33 | 2 |
| 0 | 0.40 | 0.13 | 2 | 0.35 | 0.10 | 2 | 3.19 | 0.40 | 2 |
| EC50 | 2.654 | | | 15.82 | | | 0.9213 | | |
| | G000529 B2M | | | G018995 Spy Cas | | | G022020 Spy Cas | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 96.15 | 0.07 | 2 | 98.20 | 0.42 | 2 | 6.11 | 1.70 | 2 |
| 2.5 | 94.85 | 0.49 | 2 | 97.80 | 0.00 | 2 | 6.39 | 3.83 | 2 |
| 1.25 | 93.45 | 1.20 | 2 | 97.60 | 1.13 | 2 | 3.52 | 0.04 | 2 |
| 0.625 | 90.55 | 0.07 | 2 | 96.10 | 0.42 | 2 | 2.04 | 0.66 | 2 |
| 0.313 | 83.50 | 3.39 | 2 | 91.50 | 1.84 | 2 | 1.12 | 0.07 | 2 |
| 0.078 | 57.60 | 4.67 | 2 | 75.10 | 0.00 | 2 | 0.69 | 0.16 | 2 |
| 0 | 25.25 | 1.48 | 2 | 41.20 | 0.85 | 2 | 3.00 | 2.61 | 2 |

| | | | |
|---|---|---|---|
| EC50 | 0.1087 | 0.09102 | 1.286 |

TABLE 20
Dose response curve for the percent of HLA-B7⁻ of CD8+ cells with
various doses of sgRNA

| | G028907 | | | G028913 | | | G028840 | | |
|---|---|---|---|---|---|---|---|---|---|
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 85.15 | 0.35 | 2 | 16.35 | 0.21 | 2 | 14.20 | 1.84 | 2 |
| 2.5 | 76.45 | 0.49 | 2 | 10.00 | 1.41 | 2 | 7.65 | 0.90 | 2 |
| 1.25 | 59.25 | 0.64 | 2 | 4.87 | 0.23 | 2 | 3.69 | 1.02 | 2 |
| 0.625 | 37.50 | 2.26 | 2 | 2.62 | 0.04 | 2 | 2.11 | 0.56 | 2 |
| 0.313 | 22.65 | 0.92 | 2 | 1.27 | 0.25 | 2 | 1.34 | 0.11 | 2 |
| 0.078 | 12.15 | 1.63 | 2 | 0.79 | 0.12 | 2 | 0.82 | 0.06 | 2 |
| 0 | 5.44 | 0.15 | 2 | 0.43 | 0.08 | 2 | 0.64 | 0.08 | 2 |
| EC50 | 0.8643 | | | 4.531 | | | 24.96 | | |
| | G028922 | | | G028918 | | | G028865 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 39.00 | 0.28 | 2 | 0.63 | 0.01 | 2 | 0.52 | 0.06 | 2 |
| 2.5 | 21.65 | 1.20 | 2 | 0.49 | 0.33 | 2 | 0.41 | 0.12 | 2 |
| 1.25 | 9.60 | 0.13 | 2 | 0.47 | 0.03 | 2 | 0.20 | 0.14 | 2 |
| 0.625 | 4.81 | 0.13 | 2 | 0.19 | 0.00 | 2 | 0.20 | 0.01 | 2 |
| 0.313 | 3.08 | 0.62 | 2 | 0.21 | 0.16 | 2 | 0.20 | 0.09 | 2 |
| 0.078 | 1.42 | 0.00 | 2 | 0.31 | 0.23 | 2 | 0.13 | 0.03 | 2 |
| 0 | 1.42 | 1.19 | 2 | 0.21 | 0.13 | 2 | 0.09 | 0.08 | 2 |
| EC50 | 5.494 | | | 1.217 | | | 0.03500 | | |
| | G028832 | | | G028795 | | | G028869 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 87.05 | 1.77 | 2 | 91.75 | 1.34 | 2 | 80.80 | 1.70 | 2 |
| 2.5 | 85.20 | 5.09 | 2 | 84.50 | 3.82 | 2 | 66.70 | 2.40 | 2 |
| 1.25 | 82.60 | 1.84 | 2 | 74.40 | 1.27 | 2 | 45.70 | 2.55 | 2 |
| 0.625 | 78.85 | 3.04 | 2 | 49.90 | 0.85 | 2 | 24.75 | 3.32 | 2 |
| 0.313 | 73.50 | 0.42 | 2 | 31.05 | 2.19 | 2 | 15.30 | 0.85 | 2 |
| 0.078 | 55.05 | 2.47 | 2 | 17.60 | 1.56 | 2 | 7.17 | 0.26 | 2 |
| 0 | 34.85 | 1.63 | 2 | 7.86 | 0.65 | 2 | 3.25 | 0.13 | 2 |
| EC50 | 0.1029 | | | 0.5729 | | | 1.205 | | |
| | G000529 B2M | | | G018995 Spy Cas | | | G022020 Spy Cas | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5 | 96.15 | 0.07 | 2 | 1.74 | 0.25 | 2 | 92.70 | 1.70 | 2 |
| 2.5 | 94.50 | 0.57 | 2 | 1.55 | 0.08 | 2 | 82.25 | 7.71 | 2 |
| 1.25 | 93.20 | 0.99 | 2 | 1.94 | 0.28 | 2 | 67.45 | 7.42 | 2 |
| 0.625 | 89.95 | 0.07 | 2 | 2.06 | 0.73 | 2 | 35.55 | 0.49 | 2 |
| 0.313 | 82.85 | 3.46 | 2 | 1.82 | 0.36 | 2 | 15.90 | 1.41 | 2 |
| 0.078 | 57.15 | 4.45 | 2 | 1.52 | 0.35 | 2 | 6.70 | 0.18 | 2 |
| 0 | 24.80 | 1.41 | 2 | 1.12 | 0.01 | 2 | 4.84 | 1.07 | 2 |

| | | | |
|---|---|---|---|
| EC50 | 0.1091 | 0.1053 | 0.8285 |

### Example 9. HLA-B KO in Induced Pluripotent Stem Cells (iPSC)

Induced pluripotent stem cells were edited using HLA-B guide G022020 in a single KO experiment. Additionally, iPSCs were edited using HLA-B guide G022020, HLA-A guide 018995, and CIITA guide G013675 (CCCCCGGACGGUUCAAGCAA targeting sequence; SEQ ID NO: 3110) in a triple KO experiment. The results are shown below in Table 21.

TABLE 21
Editing efficiency in iPSC using HLA-B targeting guide RNA

| | |
|---|---|
| ddPCR | Flow Cytometry |

| Efficiency | HLA-B | Efficiency | HLA-B |
|---|---|---|---|
| Single KO | 77% | Single KO | 83% |
| Triple KO | 79% | Triple KO | 71% |

### Example 10: Screening of HLA-B Guides with Nme2 BC22n

28 sgRNAs targeting HLA-B were screened at a fixed concentration of 3 μg/mL. Previously tested sgRNA (G028907) targeting HLA-B was used as a control. Guides, mRNA encoding UGI (0.5 μg/mL) (SEQ ID NO: 821), and mRNA encoding Nme2 BC22n (1 μg/mL) (SEQ ID NO: 828) were individually delivered using LNPs in parallel.

10.1 T cell Preparation

Healthy human donor apheresis was obtained commercially (Hemacare), and cells were washed and resuspended in CliniMACS® PBS/EDTA buffer (Miltenyi Biotec Cat. 130-070-525) and processed in a MultiMACS™ Cell 24 Separator Plus device (Miltenyi Biotec).
T cells were isolated via positive selection
using a Straight from Leukopak® CD4/CD8 MicroBead kit,
human (Miltenyi Biotec Cat. 130-122-352). T cells were aliquoted and cryopreserved for future use in Cryostor® CS10 (StemCell Technologies Cat. 07930). Upon thaw, T cells were plated at a density of 1.0×10⁶ cells/mL in T cell growth media (TCGM) composed of CTS OpTmizer T Cell Expansion SFM and T Cell Expansion Supplement (ThermoFisher, Cat. A1048501) containing 5% human AB serum (GeminiBio, Cat. 100-512), 1× Penicillin-Streptomycin, 1× Glutamax, 10 mM HEPES, 1× cytokines (200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15)). T cells were rested in the T cell growth media for 24 hours at which time they were activated with TransAct™ (1:100 dilution, Miltenyi Biotec, Cat. 130-111-160). T cells were activated 48 hours prior to LNP treatment.
   10.2 T Cell Editing with HTP LNPs

T cells were edited at the HLA-B locus with mRNA encoding Nme2 BC22n (SEQ ID NO: 828) and UGI (SEQ ID NO: 821) to assess sgRNA editing efficacy and the corresponding loss of HLA-B7 and HLA-B8 expression.

Forty-eight hours post activation, T cells were harvested, centrifuged at 500 g for 5 minutes, and resuspended at a concentration of 1×10⁶ T cells/mL in T cell growth media and plated in 96 well plates accordingly. For each well to be treated with LNPs, 0.5×10⁵ T cells were mixed 2:1 ratio with LNP containing 4 μg/mL of Nme2 BC22n mRNA and LNP containing 2 μg/mL of UGI mRNA and 2:1 with LNP containing 12 μg/mL HLA-B sgRNA in a final volume of 100 μL of T cell growth media. The plates were incubated at 37° C. for 10 days. On day 10 post-thaw, T cells were collected for flow cytometry analysis.

### 10.3 Flow Cytometry

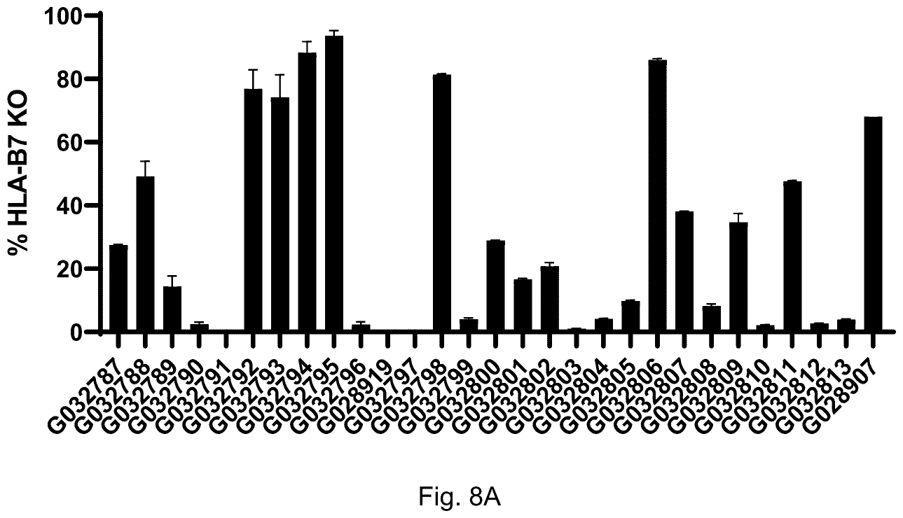

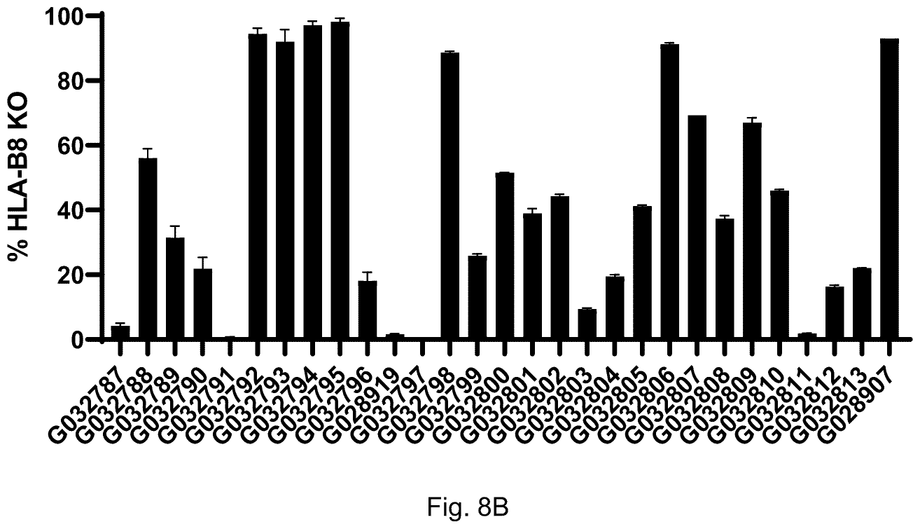

T cells were phenotyped by flow cytometry to determine HLA-B7 and HLA-B8 protein expression. Briefly, T cells were incubated for 30 minutes at 4° C. with a mixture of antibodies against CD3 (BioLegend, Cat. No. 317334), CD4 (BioLegend, Cat. No. 300536), CD8 (BioLegend, Cat. No. 344740), Viakrome (Immunotech, Cat. No. C36628), HLA-B7 (Miltenyi Biotec, Cat. No. 130-120-234), HLA-B8 (Miltenyi Biotec, Cat. No. 130-118-366), HLA-A2 (eBioscience, Cat. No. 17-9876-42), HLA-A3 (eBioscience, Cat. No. 12-5754-42). HLA-E (BioLegend, Cat. No. 342612), and HLA-C (BD Pharmingen Cat. No. 566372), diluted at 1:100 in cell staining buffer. Cells were subsequently washed and resuspended in 100 μL of cell staining buffer. Cells were then processed on a Cytoflex flow cytometer (Beckman Coulter) and analyzed using the FlowJo software package. T cells were gated based on size, shape, viability, CD8, CD3, HLA-E retention, HLA-C retention, and HLA-B7 and HLA-B8 expression. Table 22 and FIG. 8A show the mean percentage of cells negative for HLA-B7 following editing at the HLA-B locus. Table 23 and FIG. 8B show the mean percentage of cells negative for HLA-B8 following editing at the HLA-B locus.

TABLE 22
Mean % HLA-B7⁻ T cells following editing at the HLA-B locus

| | % HLA-B7 −ve | | |
|---|---|---|---|
| Guide ID | Mean | SD | N |
| G032787 | 27.46 | 0.226274 | 2 |
| G032788 | 49.24 | 4.723473 | 2 |
| G032789 | 14.4 | 3.323402 | 2 |
| G032790 | 2.48 | 0.650538 | 2 |
| G032791 | 0.035 | 0.007071 | 2 |
| G032792 | 76.87 | 5.996266 | 2 |
| G032793 | 74.19 | 7.113494 | 2 |
| G032794 | 88.33 | 3.422397 | 2 |
| G032795 | 93.61 | 1.668772 | 2 |
| G032796 | 2.365 | 0.869741 | 2 |
| G028919 | 0.165 | 0.176777 | 2 |
| G032797 | 0.125 | 0.021213 | 2 |
| G032798 | 81.355 | 0.304056 | 2 |
| G032799 | 4.075 | 0.374767 | 2 |
| G032800 | 28.985 | 0.049497 | 2 |
| G032801 | 16.63 | 0.296985 | 2 |
| G032802 | 20.805 | 1.152584 | 2 |
| G032803 | 1.035 | 0.049497 | 2 |
| G032804 | 4.1 | 0.19799 | 2 |
| G032805 | 9.765 | 0.304056 | 2 |
| G032806 | 85.975 | 0.374767 | 2 |
| G032807 | 38.13 | 0.042426 | 2 |
| G032808 | 8.245 | 0.657609 | 2 |
| G032809 | 34.675 | 2.750645 | 2 |
| G032810 | 2.105 | 0.162635 | 2 |
| G032811 | 47.61 | 0.268701 | 2 |
| G032812 | 2.72 | 0.113137 | 2 |
| G032813 | 3.965 | 0.120208 | 2 |

TABLE 23
Mean % HLA-B8⁻ T cells following editing at the HLA-B locus

| | % HLA-B8 −ve | | |
|---|---|---|---|
| Guide ID | Mean | SD | N |
| G032787 | 4.215 | 0.799031 | 2 |
| G032788 | 56.06 | 2.91328 | 2 |
| G032789 | 31.46 | 3.563818 | 2 |
| G032790 | 21.9 | 3.436539 | 2 |
| G032791 | 0.7 | 0.056569 | 2 |
| G032792 | 94.43 | 1.810193 | 2 |
| G032793 | 92.05 | 3.719382 | 2 |
| G032794 | 97.135 | 1.265721 | 2 |
| G032795 | 98.2 | 1.117229 | 2 |
| G032796 | 18.15 | 2.672864 | 2 |
| G028919 | 1.64 | 0.141421 | 2 |
| G032797 | 0.28 | 0.014142 | 2 |
| G032798 | 88.635 | 0.431335 | 2 |
| G032799 | 25.84 | 0.650538 | 2 |
| G032800 | 51.57 | 0.070711 | 2 |
| G032801 | 38.985 | 1.491995 | 2 |
| G032802 | 44.305 | 0.544472 | 2 |
| G032803 | 9.38 | 0.353553 | 2 |
| G032804 | 19.48 | 0.551543 | 2 |
| G032805 | 41.23 | 0.296985 | 2 |
| G032806 | 91.28 | 0.46669 | 2 |
| G032807 | 69.295 | 0.021213 | 2 |
| G032808 | 37.38 | 0.876812 | 2 |
| G032809 | 67.05 | 1.484924 | 2 |
| G032810 | 46.075 | 0.275772 | 2 |
| G032811 | 1.9 | 0.084853 | 2 |
| G032812 | 16.26 | 0.509117 | 2 |
| G032813 | 22.035 | 0.049497 | 2 |

### Example 11: NK Cell Functional Killing Assays

T cells edited in various combinations to disrupt CIITA, HLA-A, HLA-B, or B2M were tested for their ability to resist natural killer (NK) cell mediated killing.

### 11.1. Engineering T Cells and Purification

Upon thaw, Pan CD3+ T cells (StemCell, HLA-A*02:01:01; B*08:01:01; C*07:01:01) were plated at a density of 0.5×10⁶ cells/mL in T cell TCAM media composed of CTS OpTmizer T Cell Expansion SFM (Gibco, Cat. A3705001) containing 2.5% (v/v) of Human AB Serum, 1% (v/v) Glutamax (Gibco, Cat. 35050-061) and 1% (v/v) 1M HEPES buffer (Gibco, Cat. 15630080), 1% of Penicillin-Streptomycin, and supplemented with 100 U/mL of recombinant human interleukin-2 (Peprotech, Cat. 200-02), 5 ng/mL of human interleukin-7 (Peprotech, Cat. 200-07) and 5 ng/mL of human interleukin-15 (Peprotech, Cat. 200-15).

On Day 1 (one day post-thaw), T cells were activated with TransAct™ (1:100 dilution, Miltenyi Biotec). As described in Table 24, T cells were edited to disrupt the HLA-A, HLA-B, or B2M gene. LNP compositions containing Spy Cas9 mRNA and one sgRNA G000529 targeting B2M, sgRNA G018995 targeting HLA-A, or sgRNAs G027488, G027490, G027994, G028001, and G028002 targeting HLA-B were formulated as described in Example 1. LNP compositions were incubated in TCAM with cytokines as described above supplemented with 5 μg/ml recombinant human ApoE3 (Peprotech, Cat. 350-02) for 15 minutes at 37° C. An equal volume of LNP mix was added to one million activated T cells to yield a final concentration of 2.5 μg total LNP/mL for the B2M LNP; 1.25 μg total LNP/mL for the HLA-B LNPs, and 0.625 μg total LNP/mL for the HLA-A LNP.

TABLE 24
Order of sequential editing and viral transduction

| Condition | Day 1 | Day 2 |
|---|---|---|
| Unedited | | |
| B2M KO | B2M LNP | |
| HLA-A KO | HLA-A LNP | |
| HLA-B KO | HLA-B LNP | |
| (G027488) | | |
| HLA-B KO | HLA-B LNP | |
| (G027490) | | |
| HLA-B KO | HLA-B LNP | |
| (G027994) | | |
| HLA-B KO | HLA-B LNP | |
| (G028001) | | |
| HLA-B KO | HLA-B LNP | |
| (G028002) | | |
| HLA-A + HLA-B | HLA-A LNP | HLA-B LNP |
| KO (G027488) | | |
| HLA-A + HLA-B | HLA-A LNP | HLA-B LNP |
| KO (G027490) | | |
| HLA-A + HLA-B | HLA-A LNP | HLA-B LNP |
| KO (G027994) | | |
| HLA-A + HLA-B | HLA-A LNP | HLA-B LNP |
| KO (G028001) | | |
| HLA-A + HLA-B | HLA-A LNP | HLA-B LNP |
| KO (G028002) | | |

One day post activation (i.e., Day 2), additional T cells were edited with LNP compositions to disrupt the HLA-B gene (refer to Table 24, Day 2 column). This was performed for HLA-B editing using LNP compositions containing Spy Cas9 mRNA and sgRNAs G027488, G027490, G027994, G028001 and G028002 targeting HLA-B. LNP compositions were incubated in TCAM with cytokines as described above supplemented with 25 μg/ml recombinant human ApoE3 (Peprotech, Cat. 350-02) for 15 minutes at 37° C. A volume equal to 1/10^{th} the volume of the cells was added to each well containing approximately 1 million cells to yield a final concentration of 1.25 μg total LNP/mL for each of the HLA-B LNPs.

Two days post activation (i.e, Day 3), all cells were transferred to GREX plate (Wilson Wolf, Cat. 80240M) for expansion with TCEM media composed of CTS OpTmizer T Cell Expansion SFM (Gibco, Cat. A3705001) containing 5% (v/v) of CTS™ Immune Cell SR (Gibco, Cat. A2596101), 1% (v/v) Glutamax (Gibco, Cat. 35050-061) and 1% (v/v) 1M HEPES buffer (Gibco, Cat. 15630080), 1% of Penicillin-Streptomycin, and supplemented with 100 U/mL of recombinant human interleukin-2 (Peprotech, Cat. 200-02), 5 ng/mL of human interleukin-7 (Peprotech, Cat. 200-07) and 5 ng/mL of human interleukin-15 (Peprotech, Cat. 200-15). Half of the media from the GREX wells was replaced with fresh TCEM supplemented with cytokines on Days 5, 7 and 9.

Five days post activation (i.e., Day 6) cells were stained by flow cytometry antibodies to determine HLA-A2 expression (HLA-A+), HLA-B8 expression (HLA-B8+), and HLA-Class-I expression (MHC I+) following knockout of B2M. T cells were incubated with an antibody cocktail targeting the following molecules: HLA-A2 (Biolegend, Cat. 343326), HLA-B8 (Miltenyi Biotec, Cat. 130-118-366), and B2M (Biolegend, Cat. 316304). Cells were subsequently washed, and analyzed on a Cytoflex LX instrument (Beckman Coulter) using the FlowJo software package.

### 11.2 Flow Cytometry

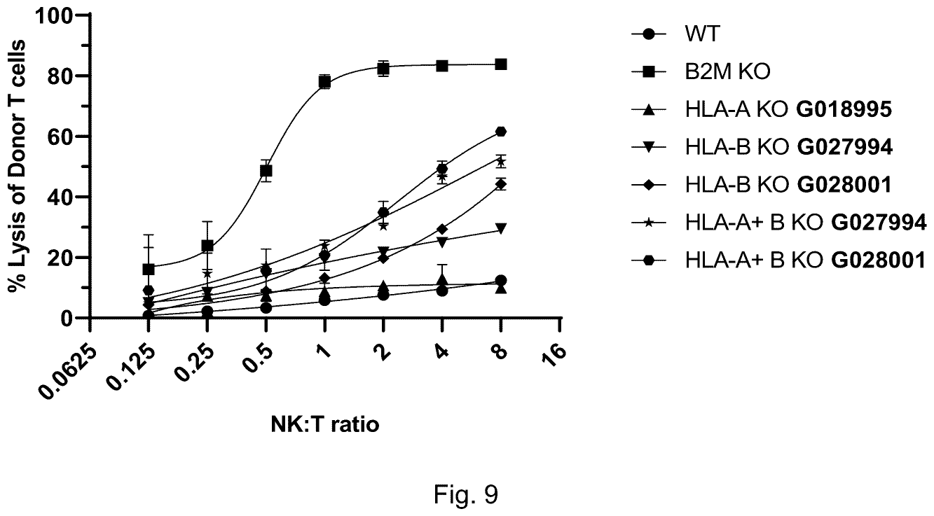

NK cell mediated cytotoxicity towards engineered T cells was assayed. T cells were co-cultured with the HLA-B/C matched CTV labelled NK cells at effector to target ratios (E:T) of 8:1, 4:1, 2:1, 1:1, 0.5:1, 0.25:1, and 0.125:1 for 21 hours. The cells were stained with 7AAD (BD Pharmingen, Cat. 559925), processed on a Cytoflex flow cytometer (Beckman Coulter) and analyzed using the FlowJo software package. T cells were gated based on CTV negativity, size, and shape and viability. Table 25 and FIG. 9 show the mean percentage of T cell lysis following NK cell challenge.

TABLE 25
Percentage T cell lysis following NK cell challenge to engineered T cells

| | | | | | HLA-A KO | | HLA-B KO | | |
|---|---|---|---|---|---|---|---|---|---|
| | WT | | B2M KO | | G018995 | | G027994 | | |
| NK:T | Mean | SD | Mean | SD | Mean | SD | Mean | SD | n |
| 8 | 12.45 | 1.272792 | 83.78 | 0.19799 | 10 | 0.565685 | 29.4 | 0.424264 | 2 |
| 4 | 8.95 | 0.141421 | 83.29 | 0.226274 | 13.1 | 4.525483 | 24.85 | 0.353553 | 2 |
| 2 | 7.65 | 0.282843 | 82.34 | 2.545584 | 10.7 | 1.131371 | 21.65 | 1.06066 | 2 |
| 1 | 5.85 | 0.707107 | 78.07 | 2.291026 | 8.8 | 2.687006 | 19.45 | 0.494975 | 2 |
| 0.5 | 3.3 | 0.919239 | 48.6 | 3.606245 | 7.45 | 1.909188 | 14.5 | 1.131371 | 2 |
| 0.25 | 2.25 | 0.282843 | 23.95 | 7.919596 | 7.25 | 0.919239 | 8.4 | 0.848528 | 2 |
| 0.125 | 0.8 | 0.212132 | 16.05 | 11.45513 | 1.25 | 0.212132 | 5.05 | 0.919239 | 2 |
| (Basal) | 0 | 0.070711 | −5.225 | 7.389266 | 0 | 1.697056 | 0 | 0 | 2 |
| 0 | | | | | | | | | |

| | HLA-B KO | | HLA-A+ B KO | | HLA-A+ B KO | | |
|---|---|---|---|---|---|---|---|
| | G028001 | | G027994 | | G028001 | | |
| NK:T | Mean | SD | Mean | SD | Mean | SD | n |
| 8 | 44.25 | 1.979899 | 51.75 | 2.12132 | 61.6 | 0.070711 | 2 |
| 4 | 29.3 | 0.212132 | 46.85 | 2.545584 | 49.25 | 2.545584 | 2 |
| 2 | 19.7 | 1.484924 | 30.35 | 1.131371 | 34.85 | 3.676955 | 2 |
| 1 | 13.25 | 1.414214 | 23.85 | 1.555635 | 20.75 | 4.949747 | 2 |
| 0.5 | 8.9 | 1.626346 | 17.4 | 0.919239 | 15.5 | 7.2832 | 2 |
| 0.25 | 2 | 0.070711 | 14.65 | 6.788225 | 0.45 | 0.707107 | 2 |
| 0.125 | 4.35 | 5.23259 | 4.9 | 1.626346 | 9.1 | 14.21285 | 2 |
| (Basal) | 0 | 2.474874 | 0 | 0.353553 | 0 | 8.980256 | 2 |
| 0 | | | | | | | |

### Example 12: LNP Dose Response Curves (DRC) for Select HLA-B Nme2 BC22n Guides

A DRC was run for select HLA-B sgRNAs along with Nme2 BC22 to determine suitable guides for knocking out the HLA-B gene. sgRNAs were titrated in 8-point DRC along with fixed concentration of an mRNA encoding UGI (SEQ ID NO: 824) (3490 ng/μL) and an mRNA encoding Nme2 BC22n base editor (SEQ ID NO: 828) (1709 ng/μL) in T cells using LNPs. T cells were then analysed by flow cytometry to determine editing efficiencies. T cells were prepared as described in Example 1.

12.1 T Cell Editing with HPLC-Purified Guide LNPs

T cells were edited at the HLA-B locus with mRNA encoding Nme2 BC22n (SEQ ID NO: 828) and UGI (SEQ ID NO: 824) to assess sgRNA editing efficacy and the corresponding loss of HLA-B7 and HLA-B8 expression.

Forty-eight hours post activation, T cells were adjusted to a concentration of 1×10⁶ T cells/mL in T cell growth media and plated in 96 well plates accordingly. For each well to be treated with LNPs, 0.5×10⁵ T cells were mixed 2:1 ratio with LNP containing 10 μg/mL of Nme2 BC22n mRNA and HLA-B sgRNA; and LNP containing 0.4 μg/mL of UGI mRNA in a final volume of 100 μL of T cell growth media. The plates were incubated at 37° C. for 10 days with every other day media refreshing. On day 10 post-thaw, T cells were collected for flow cytometry analysis.

### 12.2 Flow Cytometry

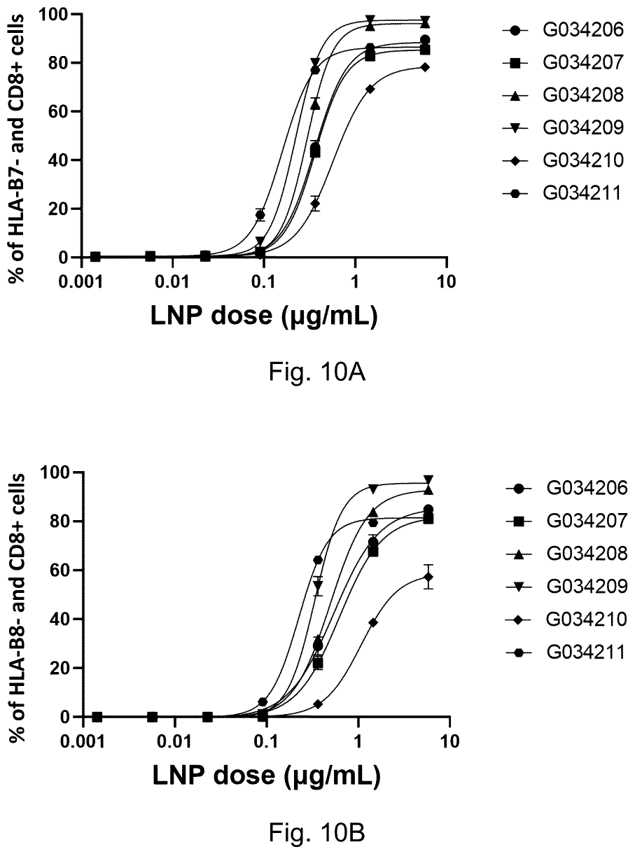

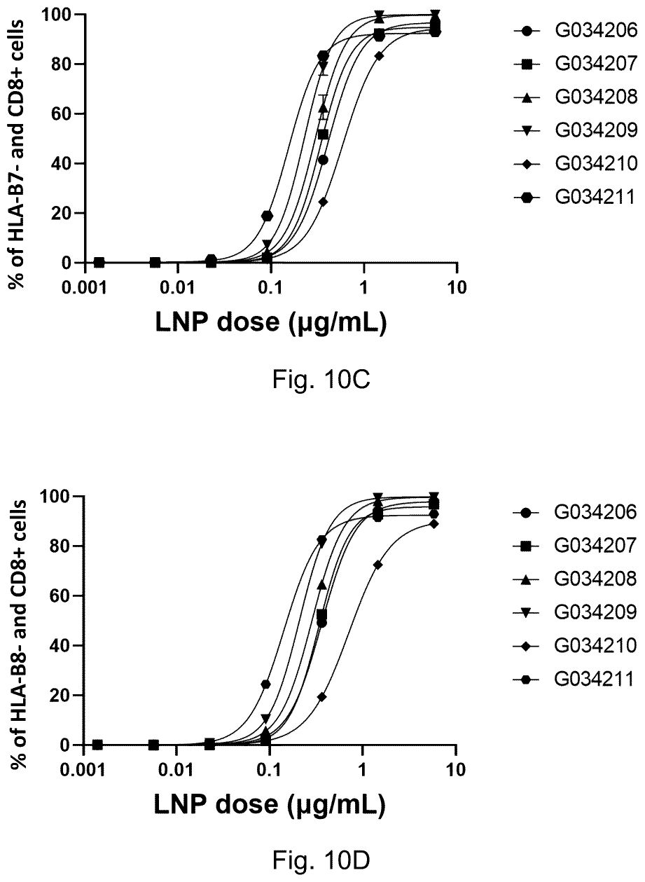

On day 10, cells were transferred to U bottom plates, spun and resuspended in master mix containing antibodies for PerCP/Cy5.5 CD3 (BioLegend, Cat. 317336), BV605 CD4 (BioLegend, Cat. 300536), BV785 CD8 (BioLegend, Cat. 344740), BV510 HLA A2 (BioLegend Inc., Cat. 343320), APC HLA A3 (eBioscience, Cat. 12-5754-42), FITC HLA B7 (Miltenyi Biotec Inc., Cat. 130-120-234), FITC HLA B8 (Miltenyi Biotec Inc., Cat. 130-118-366), and BV421 HLA-E (Biolegend, Cat. 342612), PE HLA-C (BD Pharmigen, Cat. 566372) and Viakrome (Immunotech, Cat. C36628) at 1:100 final concentration in FACs buffer and then incubated at 4° C. for 30 minutes. After the incubation, the cells were washed and resuspended in 100 μL FACs buffer (PBS+2% FBS+2 mM EDTA) and processed by flow cytometry using a Beckman Coulter CytoflexS, and analyzed using the FlowJo software package. Tables 26A and 26B and FIGS. 10A and 10C show the percent editing at each sgRNA dose in an HLA-B7 homozygous or heterozygous donor. Tables 27A and 27B and FIGS. 10B and 10D show the percent editing at each sgRNA dose in an HLA-B8 homozygous or heterozygous donor.

TABLE 26A
Dose response curve for the percent of HLA-B7⁻ and CD8⁺ cells
in an HLA-B7 homozygous donor

| | G034206 | | | G034207 | | | G034208 | | |
|---|---|---|---|---|---|---|---|---|---|
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5.83 | 89.65 | 0.13 | 2 | 85.47 | 1.44 | 2 | 96.39 | 0.17 | 2 |
| 1.46 | 84.24 | 1.87 | 2 | 82.77 | 0.01 | 2 | 95.17 | 0.13 | 2 |
| 0.36 | 45.40 | 2.73 | 2 | 43.17 | 0.74 | 2 | 63.27 | 2.40 | 2 |
| 0.09 | 1.83 | 0.42 | 2 | 2.40 | 0.08 | 2 | 2.95 | 0.49 | 2 |
| 0.02 | 0.43 | 0.10 | 2 | 0.46 | 0.06 | 2 | 0.53 | 0.19 | 2 |
| 0.005 | 0.52 | 0.18 | 2 | 0.52 | 0.32 | 2 | 0.50 | 0.43 | 2 |
| 0.001 | 0.48 | 0.15 | 2 | 0.39 | 0.11 | 2 | 0.37 | 0.23 | 2 |
| 0 | 0.43 | 0.02 | 2 | 0.30 | 0.06 | 2 | 0.49 | 0.23 | 2 |
| | G034209 | | | G034210 | | | G034211 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5.83 | 97.30 | 0.42 | 2 | 78.29 | 1.00 | 2 | 86.19 | 2.25 | 2 |
| 1.46 | 97.60 | 0.01 | 2 | 69.27 | 0.65 | 2 | 86.42 | 1.47 | 2 |
| 0.36 | 79.96 | 0.37 | 2 | 22.22 | 3.06 | 2 | 77.08 | 1.28 | 2 |
| 0.09 | 6.58 | 0.50 | 2 | 1.10 | 0.13 | 2 | 17.57 | 2.56 | 2 |
| 0.02 | 0.68 | 0.08 | 2 | 0.92 | 0.13 | 2 | 1.21 | 0.06 | 2 |
| 0.005 | 0.64 | 0.01 | 2 | 0.70 | 0.06 | 2 | 0.63 | 0.29 | 2 |
| 0.001 | 0.29 | 0.02 | 2 | 0.31 | 0.20 | 2 | 0.24 | 0.15 | 2 |
| 0 | 0.46 | 0.06 | 2 | 0.45 | 0.18 | 2 | 0.70 | 0.04 | 2 |

TABLE 26B
Dose response curve for the percent of HLA-B7⁻ and CD8⁺ cells
in an HLA-B7 heterozygous donor

| | G034206 | | | G034207 | | | G034208 | | |
|---|---|---|---|---|---|---|---|---|---|
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5.83 | 96.82 | 0.08 | 2 | 95.45 | 0.01 | 2 | 99.55 | 0.07 | 2 |
| 1.46 | 92.36 | 1.27 | 2 | 92.25 | 0.88 | 2 | 98.53 | 0.32 | 2 |
| 0.36 | 41.46 | 0.18 | 2 | 51.70 | 1.21 | 2 | 62.67 | 4.94 | 2 |
| 0.09 | 2.02 | 0.45 | 2 | 2.06 | 0.37 | 2 | 4.28 | 0.81 | 2 |
| 0.02 | 0.20 | 0.06 | 2 | 0.15 | 0.02 | 2 | 0.35 | 0.04 | 2 |
| 0.005 | 0.13 | 0.08 | 2 | 0.11 | 0.01 | 2 | 0.14 | 0.02 | 2 |
| 0.001 | 0.18 | 0.01 | 2 | 0.18 | 0.03 | 2 | 0.15 | 0.08 | 2 |
| 0 | 0.11 | 0.01 | 2 | 0.21 | 0.08 | 2 | 0.17 | 0.01 | 2 |
| | G034209 | | | G034210 | | | G034211 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5.83 | 99.84 | 0.01 | 2 | 94.04 | 0.43 | 2 | 93.08 | 0.25 | 2 |
| 1.46 | 99.52 | 0.04 | 2 | 83.31 | 0.78 | 2 | 91.28 | 0.13 | 2 |
| 0.36 | 78.88 | 3.32 | 2 | 24.51 | 0.30 | 2 | 83.39 | 1.46 | 2 |
| 0.09 | 7.19 | 1.50 | 2 | 1.46 | 0.05 | 2 | 18.89 | 1.68 | 2 |
| 0.02 | 0.28 | 0.11 | 2 | 0.30 | 0.14 | 2 | 1.32 | 0.86 | 2 |
| 0.005 | 0.23 | 0.03 | 2 | 0.27 | 0.01 | 2 | 0.21 | 0.00 | 2 |
| 0.001 | 0.14 | 0.07 | 2 | 0.24 | 0.02 | 2 | 0.18 | 0.01 | 2 |
| 0 | 0.17 | 0.05 | 2 | 0.22 | 0.06 | 2 | 0.18 | 0.06 | 2 |

TABLE 27A
Dose response curve for the percent of HLA-B8⁻ and CD8⁺ cells
in an HLA-B8 homozygous donor

| | G034206 | | | G034207 | | | G034208 | | |
|---|---|---|---|---|---|---|---|---|---|
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5.83 | 84.88 | 1.32 | 2 | 80.95 | 0.42 | 2 | 92.87 | 0.18 | 2 |
| 1.46 | 71.55 | 2.94 | 2 | 67.64 | 1.03 | 2 | 83.77 | 1.66 | 2 |
| 0.36 | 29.04 | 3.68 | 2 | 22.14 | 2.69 | 2 | 31.88 | 0.41 | 2 |
| 0.09 | 0.29 | 0.10 | 2 | 0.21 | 0.06 | 2 | 0.40 | 0.04 | 2 |
| 0.02 | 0.01 | 0.00 | 2 | 0.01 | 0.01 | 2 | 0.00 | 0.00 | 2 |
| 0.005 | 0.01 | 0.01 | 2 | 0.02 | 0.03 | 2 | 0.02 | 0.02 | 2 |
| 0.001 | 0.01 | 0.01 | 2 | 0.00 | 0.00 | 2 | 0.00 | 0.00 | 2 |
| 0 | 0.01 | 0.00 | 2 | 0.00 | 0.00 | 2 | 0.05 | 0.01 | 2 |
| | G034209 | | | G034210 | | | G034211 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5.83 | 96.75 | 0.34 | 2 | 57.29 | 4.96 | 2 | 82.85 | 0.35 | 2 |
| 1.46 | 93.01 | 0.52 | 2 | 38.65 | 0.18 | 2 | 79.35 | 0.54 | 2 |
| 0.36 | 53.42 | 3.96 | 2 | 5.28 | 0.22 | 2 | 64.19 | 0.30 | 2 |
| 0.09 | 1.17 | 0.16 | 2 | 0.04 | 0.01 | 2 | 6.25 | 0.87 | 2 |
| 0.02 | 0.03 | 0.01 | 2 | 0.02 | 0.01 | 2 | 0.09 | 0.07 | 2 |
| 0.005 | 0.01 | 0.01 | 2 | 0.02 | 0.03 | 2 | 0.02 | 0.01 | 2 |
| 0.001 | 0.00 | 0.00 | 2 | 0.01 | 0.00 | 2 | 0.02 | 0.01 | 2 |
| 0 | 0.00 | 0.00 | 2 | 0.00 | 0.00 | 2 | 0.01 | 0.01 | 2 |

TABLE 27B
Dose response curve for the percent of HLA-B8⁻ and CD8⁺ cells
in an HLA-B8 heterozygous donor

| | G034206 | | | G034207 | | | G034208 | | |
|---|---|---|---|---|---|---|---|---|---|
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5.83 | 97.98 | 0.23 | 2 | 96.75 | 0.55 | 2 | 99.44 | 0.18 | 2 |
| 1.46 | 94.16 | 0.66 | 2 | 92.78 | 0.26 | 2 | 98.16 | 0.11 | 2 |
| 0.36 | 49.19 | 1.39 | 2 | 52.56 | 0.23 | 2 | 64.63 | 0.85 | 2 |
| 0.09 | 3.25 | 0.48 | 2 | 1.89 | 0.11 | 2 | 5.70 | 0.64 | 2 |
| 0.02 | 0.19 | 0.04 | 2 | 0.02 | 0.00 | 2 | 0.24 | 0.14 | 2 |
| 0.005 | 0.11 | 0.06 | 2 | 0.04 | 0.01 | 2 | 0.07 | 0.07 | 2 |
| 0.001 | 0.07 | 0.04 | 2 | 0.10 | 0.02 | 2 | 0.12 | 0.00 | 2 |
| 0 | 0.14 | 0.01 | 2 | 0.17 | 0.11 | 2 | 0.16 | 0.05 | 2 |
| | G034209 | | | G034210 | | | G034211 | | |
| Guide | Mean | SD | | Mean | SD | | Mean | SD | |
| sgRNA | % CD8⁺ | % CD8⁺ | | % CD8⁺ | % CD8⁺ | | % CD8⁺ | % CD8⁺ | |
| (μg/mL) | Cells | Cells | N | Cells | Cells | N | Cells | Cells | N |
| 5.83 | 99.66 | 0.11 | 2 | 88.96 | 0.16 | 2 | 92.88 | 0.25 | 2 |
| 1.46 | 99.31 | 0.04 | 2 | 72.50 | 0.57 | 2 | 91.49 | 0.72 | 2 |
| 0.36 | 80.98 | 0.74 | 2 | 19.39 | 1.07 | 2 | 82.62 | 0.29 | 2 |
| 0.09 | 10.34 | 0.35 | 2 | 1.59 | 0.06 | 2 | 24.49 | 0.51 | 2 |
| 0.02 | 0.79 | 0.52 | 2 | 0.14 | 0.03 | 2 | 0.74 | 0.07 | 2 |
| 0.005 | 0.13 | 0.13 | 2 | 0.14 | 0.01 | 2 | 0.13 | 0.05 | 2 |
| 0.001 | 0.11 | 0.06 | 2 | 0.11 | 0.03 | 2 | 0.10 | 0.06 | 2 |
| 0 | 0.14 | 0.01 | 2 | 0.17 | 0.11 | 2 | 0.16 | 0.05 | 2 |

### Example 13: Off-Target Analysis of HLA-B Human Guides

Screening for potential off-target genomic sites cleaved by Cas9 targeting HLA-B was performed. (See, e.g., Cameron et al., Nature Methods. 6, 600-606; 2017). In this experiment, 6 sgRNA targeting human HLA-B and three control guides targeting VEGFA with known off-target profiles were screened using purified genomic DNA from lymphoblast cell line NA24385 (Coriell Institute). The number of potential off-target sites were detected using a sgRNA as shown in Table 28 at a concentration of 192 nM sgRNA and 64 nM RNP in the biochemical assay. The assay identified potential off-target sites for the sgRNAs tested.

TABLE 28
Off-Target Analysis

| | | | Off- |
|---|---|---|---|
| | | | Target |
| gRNA | | Guide Sequence | Site |
| ID | Target | (SEQ ID NO:) | Count |
| G034206 | HLA-B | CAAACUCAGGACACUGAGCUUGUG | 1 |
| | | (SEQ ID NO: 163) | |
| G034207 | HLA-B | UCAGGACACUGAGCUUGUGGAGAC | 1 |
| | | (SEQ ID NO: 164) | |
| G034208 | HLA-B | UCUGGGAAAGGAGGGGAAGAUGAG | 1 |
| | | (SEQ ID NO: 165) | |
| G034209 | HLA-B | CUCUGGGAAAGGAGGGGAAGAUGA | 1 |
| | | (SEQ ID NO: 166) | |
| G034210 | HLA-B | CUGGAGGGUGUGAGACCCUGGCCC | 3 |
| | | (SEQ ID NO: 169) | |
| G034211 | HLA-B | UCCCAGAGCCGUCUUCCCAGUCCA | 2 |
| | | (SEQ ID NO: 177) | |
| G021557 | VEGFA | GCAUGGGCAGGGGCUGGGGUGCAC | 2 |
| | | (SEQ ID NO: 610) | |
| G021558 | VEGFA | GAAUGGCAGGCGGAGGUUGUACUG | 1 |
| | | (SEQ ID NO: 611) | |
| G021567 | VEGFA | GUGAGCAGGCACCUGUGCCAACAU | 1 |
| | | (SEQ ID NO: 612) | |

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically overrepresents the number of potential off-target sites as the assay utilizes purified high molecular weight genomic DNA free of the cell environment and is dependent on the dose of Cas9 RNP used. Accordingly, potential off-target sites identified by these methods may be validated using targeted sequencing of the identified potential off-target sites.

### Example 14: In Vivo NK Cell Killing of Engineered T Cells in a Mouse Model

Female NOG-hIL-15 mice were engrafted with 1.5×10⁶ primary NK cells. Engineered T cells containing luciferase and edited according to Table 29 (various combinations of B2M/HLA-A/HLA-B/TRAC/CIITA KO) were injected 4 weeks later in order to assess protection of the engineered T cells from NK cell killing. 14.1. Preparation of T cells containing luciferase +/−B2M/HLA-A/HLA-B/TRAC/CIITA KO and HD1 TCR

On day 0, upon thaw, Pan CD3+ T cells (StemCell, HLA-A*02:01:01 A*11:01:01; B*08:01:01; C*07:01:01) were plated at a density of 1-1.5×10⁶ cells/mL in T cell TCAM media composed of CTS OpTmizer T Cell Expansion SFM (Gibco, Cat. A3705001) containing 2.5% (v/v) of Human AB Serum, 1% (v/v) Glutamax (Gibco, Cat. 35050-061) and 1% (v/v) 10 1M HEPES buffer (Gibco, Cat. 15630080), 1% of Penicillin-Streptomycin, and supplemented with 200 U/mL of recombinant human interleukin-2 (Peprotech, Cat. 200-02), 5 ng/mL of human interleukin-7 (Peprotech, Cat. 200-07) and 5 ng/mL of human interleukin-15 (Peprotech, Cat. 200-15).

On day 1, T cells were activated with TransAct™ (1:100 dilution, Miltenyi Biotec).

On day 2, cells were transduced with a Lentivirus—LV-SFFV-Luc2-P2A-EmGFP to express GFP/Firefly luciferase (Imanis, Cat. LV050L) at a MOI of 0.5. This was performed by centrifuging the T cells at 500×g for 5 minutes, resuspending them at 2E6 cells/mL and transferring them to sterile 1.5 mL Eppendorf tubes such that each tube received 1E6 cells in 0.5 mL. 100 μL of LV-SFFV-Luc2-P2A-EmGFP (Imanis, Cat. LV050L) was added to each tube and the transduction was performed by centrifuging the tubes at 1000×g for 60 minutes at 37° C. Post centrifugation, cells were combined and transferred back to a cell culture flask and rested overnight in a 37° C. incubator.

On day 3, as described in Table 29, T cells were edited with LNPs to disrupt the HLA-A or CIITA genes. Briefly, LNPs for each of BC22 mRNA, UGI mRNA and sgRNA G028918 targeting HLA-A or sgRNA G026584 targeting CIITA were formulated as described in Example 1. LNP compositions were incubated in TCAM with cytokines as described above supplemented with 20 μg/mL recombinant human ApoE3 (Peprotech, Cat. 350-02). An equal volume of LNP mix was added to one million activated T cells to yield a final concentration of 0.266 μg total LNP/mL for the HLA-A LNP and 0.34 μg total LNP/mL for the CIITA LNP.

TABLE 29
Order of sequential editing and viral transduction

| Condition | Day 3 | Day 4 | Day 8 |
|---|---|---|---|
| Unedited | | | |
| B2M + CIITA KO | CIITA LNP | B2M LNP | TRAC LNP |
| HLA-A + CIITA | HLA-A LNP, | | TRAC LNP |
| | CIITA LNP | | |
| HLA-B + CIITA | CIITA LNP | HLA-B LNP | TRAC LNP |
| HLA-A + HLA- | HLA-A LNP, | | TRAC LNP |
| B + CIITA | CIITA LNP | | |

On day 4, the T cells from each group were counted, re-plated at 2×10⁶ cells/mL and edited with LNP compositions to disrupt the HLA-B or B2M genes (where noted in Table 29, Day 4 column). This was performed with LNPs co-formulated with either Nme2 BC22n mRNA and sgRNA G032795 targeting the HLA-B gene or Spy Cas9 mRNA and sgRNA G000529 targeting the B2M gene. LNP compositions were incubated in TCAM with cytokines as described above supplemented with 5 μg/mL recombinant human ApoE3 (Peprotech, Cat. 350-02). A volume equal to the volume of the cells was added to each well containing ˜2 million cells to yield a final concentration of 1.25 μg total LNP/mL for the HLA-B LNP and 2.5 μg total LNP/mL for the B2M LNP.

On day 5, all cells were transferred to GREX plate (Wilson Wolf, Cat. 80240M) for expansion with TCAM media composed of CTS OpTmizer T Cell Expansion SFM (Gibco, Cat. A3705001) containing 2.5% (v/v) of Human AB Serum, 1% (v/v) Glutamax (Gibco, Cat. 35050-061) and 1% (v/v) 10 1M HEPES buffer (Gibco, Cat. 15630080), 1% of Penicillin-Streptomycin, and supplemented with 100 U/mL of recombinant human interleukin-2 (Peprotech, Cat. 200-02), 5 ng/mL of human interleukin-7 (Peprotech, Cat. 200-07) and 5 ng/mL of human interleukin-15 (Peprotech, Cat. 200-15).

On day 7, each of the T cell groups was sorted on the GFP+ population, and cells were put back in culture in TCAM media composed of CTS OpTmizer T Cell Expansion SFM (Gibco, Cat. A3705001) containing 2.5% (v/v) of Human AB Serum, 1% (v/v) Glutamax (Gibco, Cat. 35050-061) and 1% (v/v) 10 1M HEPES buffer (Gibco, Cat. 15630080), 1% of Penicillin-Streptomycin, and supplemented with 100 U/mL of recombinant human interleukin-2 (Peprotech, Cat. 200-02), 5 ng/mL of human interleukin-7 (Peprotech, Cat. 200-07) and 5 ng/mL of human interleukin-15 (Peprotech, Cat. 200-15).

On day 8, sorted cells were activated with TransAct™ (1:100 dilution, Miltenyi Biotec), and edited with LNP formulations to disrupt the TRAC gene. Briefly, LNPs for each Nme2 BC22 mRNA (mRNA100418), UGI mRNA (mRNA100032) and sgRNA G028939 targeting TRAC were formulated as described in Example 1. LNP compositions were incubated in TCAM with cytokines as described above supplemented with 20 μg/mL recombinant human ApoE3 (Peprotech, Cat. 350-02). An equal volume of LNP mix was added to one million activated T cells to yield a final concentration of 0.209 μg total LNP/mL for the TRAC LNP.

On day 10, all cells were transferred to GREX plate (Wilson Wolf, Cat. 80240M) for expansion with TCAM media composed of CTS OpTmizer T Cell Expansion SFM (Gibco, Cat. A3705001) containing 2.5% (v/v) of Human AB Serum, 1% (v/v) Glutamax (Gibco, Cat. 35050-061) and 1% (v/v) 10 1M HEPES buffer (Gibco, Cat. 15630080), 1% of Penicillin-Streptomycin, and supplemented with 100 U/mL of recombinant human interleukin-2 (Peprotech, Cat. 200-02), 5 ng/mL of human interleukin-7 (Peprotech, Cat. 200-07) and 5 ng/mL of human interleukin-15 (Peprotech, Cat. 200-15).

Half of the media from the GREX wells was replaced with fresh TCEM supplemented with cytokines on Days 12 and 14.

On day 17, an aliquot of cells was stained by flow cytometry antibodies to determine HLA-A2 expression (HLA-A+), HLA-B8 expression (HLA-B8+), HLA-Class-I expression (MHC I+) following knockout of B2M, HLA-Class-II expression (MHC II+) following knockout of CIITA, and CD3 expression following knockout of TRAC. T cells were incubated with an antibody cocktail targeting the following molecules: HLA-A2 (B eBioscience, Cat. 17-9876-42), HLA-B8 (Miltenyi Biotec, Cat. 130-118-960), CD3 (Biolegend, Cat. 317334), CD4 (Biolegend, Cat. 300536), CD8 (Biolegend, Cat. 344740), HLA-DR, DP, DQ (HLA Class-II) (Biolegend, Cat. 361712), HLA-E (Biolegend, Cat. 342612), and ViaKrome 808 Fixable Viability Dye (Beckman Coulter, Cat. C36628). Cells were subsequently washed, analyzed on a Cytoflex LX instrument (Beckman Coulter) using the FlowJo software package. The rest of the cells were frozen down for subsequent T cell injections in mice using a 1:1 dilution for CSB (Stemcells, 100-0237) and CS10 (Stemcells, 7930).

14.2. HLA-A and/or HLA-B with CIITA Knockout T Cells Show Greater Protection from NK Killing than B2M/CIITA KO T Cells

NK cells were isolated from a leukopak by methods known in the art, washed with HBSS (Gibco, Cat. No. 14025-092) and resuspended at 10×10⁶ cells/mL for injection in 150 μL HBSS. Thirty female NOG-hIL-15 mice (Taconic) were dosed by tail vein injection with 1.5×10⁶ isolated NK cells. An additional twenty-five female NOG-hIL-15 served NK-non-injected controls.

Twenty-seven days after NK cell injection, mice were injected with unedited or engineered T cells as described in Table 29. Briefly, engineered T cells were injected 16 days post first activation after washing in PBS and resuspending in HBSS solution at a concentration of 6.0×10⁶ cells/150 μL.

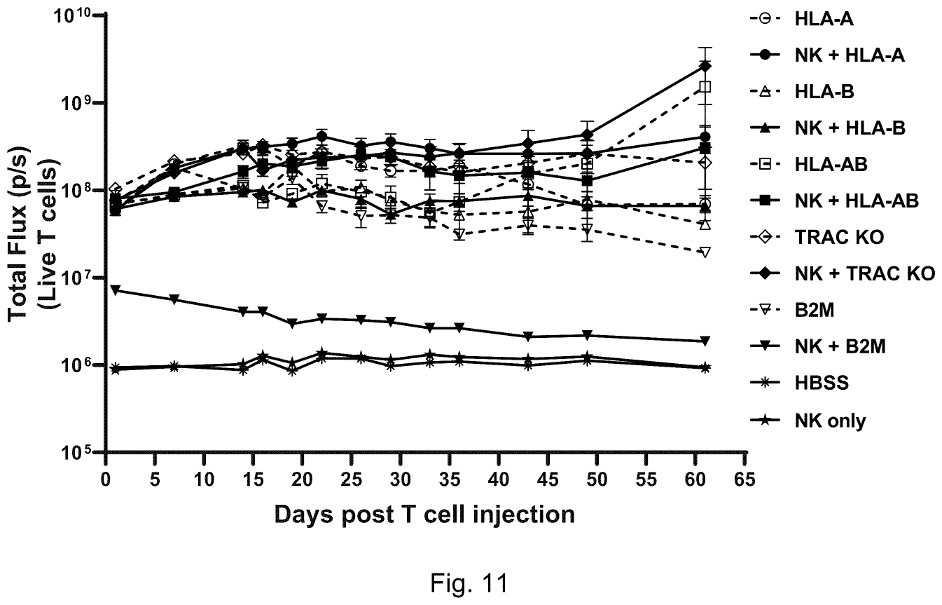

IVIS imaging of live mice was performed to identify luciferase-positive T cells by IVIS spectrum. IVIS imaging was done at 24 hours, 7 days, 14 days, 16 days, 19 days, 22 days, 26 days, 29 days, 33 days, 36 days, 43 days, 49 days, and 61 days after T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 30 and FIG. 11 shows total flux (photons/s) from luciferase expressing T cells present at the various time points after injection. In vivo, B32M edited cells showed sensitivity to NK killing, while HLA-A, HLA-B, TRAC and CIITA edited cells (as shown in Table 29) showed protection from NK mediated lysis.

TABLE 30
Total Flux (photons/s) from luciferase expressing T cells in
treated mice at intervals after T cell injection.

| | Days | | | | | | |
|---|---|---|---|---|---|---|---|
| | post | | | | | | |
| | T cell | | | | | | |
| | injec- | No NK injection | | | NK injection | | |
| Group | tion | Mean | SD | N | Mean | SD | N |
| TRAC | 1 | 1.03E+08 | 1.70E+07 | 4 | 7.70E+07 | 1.04E+07 | 5 |
| KO | 7 | 2.15E+08 | 1.36E+07 | 4 | 1.57E+08 | 2.85E+07 | 5 |
| | 14 | 2.66E+08 | 4.04E+07 | 4 | 3.01E+08 | 5.64E+07 | 5 |
| | 16 | 3.27E+08 | 4.84E+07 | 4 | 1.69E+08 | 4.40E+07 | 5 |
| | 19 | 2.57E+08 | 5.99E+07 | 4 | 2.22E+08 | 7.06E+07 | 5 |
| | 22 | 2.74E+08 | 3.40E+07 | 4 | 2.38E+08 | 1.19E+08 | 5 |
| | 26 | 2.33E+08 | 3.74E+07 | 4 | 2.45E+08 | 1.45E+08 | 5 |
| | 29 | 2.39E+08 | 5.41E+07 | 4 | 2.70E+08 | 1.46E+08 | 5 |
| | 33 | 1.85E+08 | 4.72E+07 | 4 | 2.42E+08 | 1.47E+08 | 5 |
| | 36 | 1.62E+08 | 1.71E+07 | 4 | 2.67E+08 | 1.51E+08 | 5 |
| | 43 | 2.05E+08 | 6.97E+07 | 4 | 3.45E+08 | 2.75E+08 | 5 |
| | 49 | 2.62E+08 | 1.85E+08 | 4 | 4.34E+08 | 3.66E+08 | 5 |
| | 61 | 2.07E+08 | 1.80E+08 | 4 | 2.63E+09 | 3.35E+09 | 5 |
| B2M | 1 | 7.23E+07 | 1.05E+07 | 4 | 7.14E+06 | 1.44E+06 | 5 |
| | 7 | 8.40E+07 | 1.00E+07 | 4 | 5.57E+06 | 7.80E+05 | 5 |
| | 14 | 1.07E+08 | 1.65E+07 | 4 | 4.06E+06 | 6.93E+05 | 5 |
| | 16 | 8.41E+07 | 2.04E+07 | 4 | 4.05E+06 | 5.02E+05 | 5 |
| | 19 | 1.38E+08 | 3.78E+07 | 4 | 2.96E+06 | 2.46E+05 | 5 |
| | 22 | 6.59E+07 | 1.77E+07 | 4 | 3.38E+06 | 4.43E+05 | 5 |
| | 26 | 5.09E+07 | 2.33E+07 | 4 | 3.26E+06 | 4.57E+05 | 5 |
| | 29 | 5.20E+07 | 1.76E+07 | 4 | 3.11E+06 | 5.11E+05 | 5 |
| | 33 | 4.86E+07 | 1.75E+07 | 4 | 2.65E+06 | 2.23E+05 | 5 |
| | 36 | 3.13E+07 | 7.28E+06 | 4 | 2.64E+06 | 2.87E+05 | 5 |
| | 43 | 3.96E+07 | 1.47E+07 | 4 | 2.10E+06 | 2.80E+05 | 5 |
| | 49 | 3.56E+07 | 3.23E+06 | 4 | 2.18E+06 | 2.29E+05 | 5 |
| | 61 | 1.94E+07 | 3.89E+06 | 4 | 1.86E+06 | 2.74E+05 | 5 |
| HLA-A | 1 | 5.93E+07 | 5.19E+06 | 4 | 5.92E+07 | 1.01E+07 | 5 |
| | 7 | 1.99E+08 | 2.97E+07 | 4 | 1.80E+08 | 4.31E+07 | 5 |
| | 14 | 3.23E+08 | 8.66E+07 | 4 | 3.01E+08 | 9.32E+07 | 5 |
| | 16 | 3.05E+08 | 9.72E+07 | 4 | 3.13E+08 | 8.01E+07 | 5 |
| | 19 | 1.88E+08 | 3.55E+07 | 4 | 3.41E+08 | 1.10E+08 | 5 |
| | 22 | 2.75E+08 | 2.41E+07 | 4 | 4.14E+08 | 1.68E+08 | 5 |
| | 26 | 1.89E+08 | 3.99E+07 | 4 | 3.24E+08 | 1.40E+08 | 5 |
| | 29 | 1.67E+08 | 4.29E+07 | 4 | 3.60E+08 | 1.65E+08 | 5 |
| | 33 | 1.67E+08 | 4.25E+07 | 4 | 3.03E+08 | 1.59E+08 | 5 |
| | 36 | 1.91E+08 | 5.23E+07 | 4 | 2.63E+08 | 1.43E+08 | 5 |
| | 43 | 1.17E+08 | 2.24E+07 | 4 | 2.62E+08 | 1.50E+08 | 5 |
| | 49 | 6.79E+07 | 1.14E+07 | 4 | 2.64E+08 | 1.27E+08 | 5 |
| | 61 | 6.99E+07 | 2.02E+07 | 4 | 4.09E+08 | 2.92E+08 | 5 |
| HLA-B | 1 | 7.56E+07 | 4.17E+07 | 4 | 6.13E+07 | 8.43E+06 | 5 |
| | 7 | 1.81E+08 | 1.85E+07 | 4 | 8.47E+07 | 1.53E+07 | 5 |
| | 14 | 1.04E+08 | 9.88E+06 | 4 | 9.54E+07 | 8.16E+06 | 5 |
| | 16 | 9.11E+07 | 5.68E+06 | 4 | 1.01E+08 | 2.80E+07 | 5 |
| | 19 | 1.90E+08 | 2.08E+07 | 4 | 7.30E+07 | 1.48E+07 | 5 |
| | 22 | 9.67E+07 | 3.17E+06 | 4 | 1.00E+08 | 2.37E+07 | 5 |
| | 26 | 1.08E+08 | 2.21E+07 | 4 | 7.96E+07 | 2.17E+07 | 5 |
| | 29 | 7.63E+07 | 1.59E+07 | 4 | 5.30E+07 | 8.34E+06 | 5 |
| | 33 | 5.77E+07 | 1.32E+07 | 4 | 7.60E+07 | 2.91E+07 | 5 |
| | 36 | 5.23E+07 | 9.16E+06 | 4 | 7.50E+07 | 3.48E+07 | 5 |
| | 43 | 5.72E+07 | 8.07E+06 | 4 | 8.64E+07 | 4.06E+07 | 5 |
| | 49 | 7.81E+07 | 3.27E+07 | 4 | 6.60E+07 | 3.65E+07 | 5 |
| | 61 | 4.10E+07 | 7.65E+06 | 4 | 6.60E+07 | 4.21E+07 | 5 |
| HLA- | 1 | 6.89E+07 | 5.43E+06 | 4 | 8.32E+07 | 6.69E+06 | 5 |
| AB | 7 | 8.96E+07 | 8.11E+06 | 4 | 1.53E+08 | 1.17E+08 | 5 |
| | 14 | 1.13E+08 | 1.06E+07 | 4 | 4.65E+08 | 6.03E+08 | 5 |
| | 16 | 7.28E+07 | 1.70E+07 | 4 | 6.94E+08 | 9.88E+08 | 5 |
| | 19 | 9.11E+07 | 2.35E+07 | 4 | 1.27E+09 | 2.15E+09 | 5 |
| | 22 | 1.20E+08 | 5.52E+07 | 4 | 2.58E+09 | 4.72E+09 | 5 |
| | 26 | 9.65E+07 | 5.94E+07 | 4 | 2.95E+09 | 5.40E+09 | 5 |
| | 29 | 8.27E+07 | 5.16E+07 | 4 | 3.10E+09 | 5.72E+09 | 5 |
| | 33 | 5.52E+07 | 3.10E+07 | 4 | 1.66E+08 | 1.11E+08 | 5 |
| | 36 | 7.34E+07 | 8.04E+07 | 4 | 1.47E+08 | 9.66E+07 | 5 |
| | 43 | 1.55E+08 | 2.13E+08 | 4 | 1.60E+08 | 9.99E+07 | 5 |
| | 49 | 2.02E+08 | 3.05E+08 | 4 | 1.29E+08 | 5.71E+07 | 5 |
| | 61 | 1.53E+09 | 2.56E+09 | 4 | 3.08E+08 | 3.14E+08 | 5 |
| NK only | 1 | | | | 8.88E+05 | 8.15E+04 | 5 |
| | 7 | | | | 9.56E+05 | 4.69E+04 | 5 |
| | 14 | | | | 1.03E+06 | 9.95E+04 | 5 |
| | 16 | | | | 1.28E+06 | 1.22E+05 | 5 |
| | 19 | | | | 1.06E+06 | 5.24E+04 | 5 |
| | 22 | | | | 1.38E+06 | 1.30E+05 | 5 |
| | 26 | | | | 1.24E+06 | 1.03E+05 | 5 |
| | 29 | | | | 1.14E+06 | 1.48E+05 | 5 |
| | 33 | | | | 1.32E+06 | 1.81E+05 | 5 |
| | 36 | | | | 1.24E+06 | 8.79E+04 | 5 |
| | 43 | | | | 1.17E+06 | 9.95E+04 | 5 |
| | 49 | | | | 1.25E+06 | 1.65E+05 | 5 |
| | 61 | | | | 9.40E+05 | 3.32E+04 | 5 |
| HBSS | 1 | 9.41E+05 | 7.08E+04 | 5 | | | |
| | 7 | 9.77E+05 | 9.09E+04 | 5 | | | |
| | 14 | 8.80E+05 | 4.23E+04 | 5 | | | |
| | 16 | 1.15E+06 | 7.44E+04 | 5 | | | |
| | 19 | 8.56E+05 | 1.00E+05 | 5 | | | |
| | 22 | 1.19E+06 | 2.02E+05 | 5 | | | |
| | 26 | 1.19E+06 | 9.09E+04 | 5 | | | |
| | 29 | 9.74E+05 | 8.72E+04 | 5 | | | |
| | 33 | 1.07E+06 | 6.09E+04 | 5 | | | |
| | 36 | 1.10E+06 | 8.36E+04 | 5 | | | |
| | 43 | 9.89E+05 | 5.35E+04 | 5 | | | |
| | 49 | 1.12E+06 | 1.46E+05 | 5 | | | |
| | 61 | 9.24E+05 | 7.21E+04 | 5 | | | |

### Example 15: In Vivo NK Cell Killing of Engineered T Cells in a Mouse Model

Female NOG-hIL-15 mice were engrafted with 1.5×10⁶ primary NKI cells. Engineered T cells containing luciferase and edited according to Table 29 (various combinations of HLA-A/HLA-B/TRAC/CIITA KO) were injected 4 weeks later in order to assess protection of the engineered T cells from NKI cell killing.

### 15.1. Preparation of T Cells Containing Luciferase +/−B2M/HLA-A/HLA-B/TRAC/CIITA KO and HD1 TCR

T cells were prepared as in Example 14.1 except on day 4, the edit was performed with LNPs co-formulated with Spy Cas9 mRNA gRNA G027994 targeting the HLA-B gene.

15.2. HLA-A and/or HLA-B with CIITA Knockout T Cells Show Greater Protection from NK Killing than B2M/CIITA KO T Cells

The NK cells and mice were prepared as in Example 14.2.

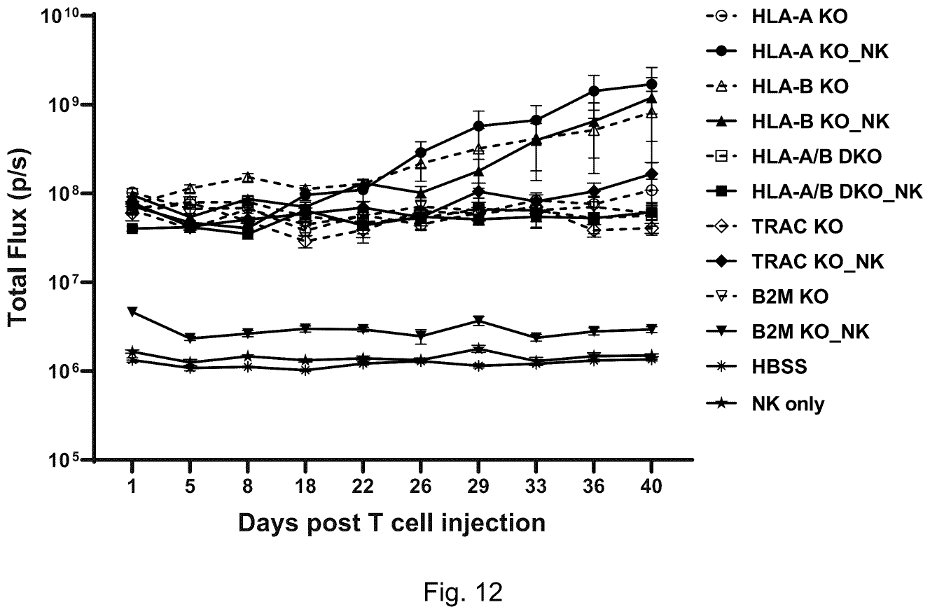

IVIS imaging of live mice was performed to identify luciferase-positive T cells by IVIS spectrum. IVIS imaging was done at 24 hours, 7 days, 14 days, 16 days, 19 days, 22 days, 26 days, 29 days, 33 days, 36 days, and 40 days after T cell injection. Mice were prepared for imaging as in Example 14.2. Table 31 and FIG. 12 shows total flux (photons/s) from luciferase expressing T cells present at the various time points after injection. In vivo, B32M edited cells showed sensitivity to NK killing, while HLA-A, HLA-B, TRAC and CIITA edited cells (as shown in Table 29) showed protection from NK mediated lysis.

TABLE 31
Total Flux (photons/s) from luciferase expressing T cells in treated
mice at indicated days after T cell injection.

| | | No NK injection | | | NK injection | | |
|---|---|---|---|---|---|---|---|
| Group | Days | Mean | SD | N | Mean | SD | N |
| TRAC | 1 | 60237500 | 19287091 | 4 | 78610000 | 5481726 | 5 |
| KO | 5 | 79142500 | 21375333 | 4 | 42522000 | 7716803 | 5 |
| | 8 | 48687500 | 4504023 | 4 | 50922000 | 5762317 | 5 |
| | 18 | 28987500 | 7896542 | 4 | 59024000 | 19425221 | 5 |
| | 22 | 38695000 | 11931246 | 4 | 69482000 | 23737977 | 5 |
| | 26 | 62917500 | 12062851 | 4 | 54070000 | 21862370 | 5 |
| | 29 | 59235000 | 17354556 | 4 | 1.05E+08 | 51444384 | 5 |
| | 33 | 70220000 | 18308445 | 4 | 81094000 | 30428047 | 5 |
| | 36 | 38415000 | 10687349 | 4 | 1.06E+08 | 50005246 | 5 |
| | 40 | 40777500 | 8983302 | 4 | 1.66E+08 | 1.14E+08 | 5 |
| B2M | 1 | 61697500 | 3710178 | 4 | 4625200 | 1017123 | 5 |
| | 5 | 40126000 | 7705511 | 4 | 2337400 | 244229.9 | 5 |
| | 8 | 66758000 | 14162942 | 4 | 2650600 | 417803.6 | 5 |
| | 18 | 44676000 | 16960004 | 4 | 2997800 | 477095.1 | 5 |
| | 22 | 56008000 | 25277849 | 4 | 2953600 | 344565 | 5 |
| | 26 | 71536000 | 36732385 | 4 | 2469720 | 916718.8 | 5 |
| | 29 | 64236000 | 29804451 | 4 | 3688200 | 846342.1 | 5 |
| | 33 | 64786000 | 33498350 | 4 | 2378200 | 408716.7 | 5 |
| | 36 | 70582000 | 38523286 | 4 | 2803200 | 512631 | 5 |
| | 40 | 59696000 | 33547829 | 4 | 2952000 | 459698.6 | 5 |
| HLA-A | 1 | 1.03E+08 | 18126125 | 4 | 72340000 | 10886537 | 5 |
| | 5 | 67105000 | 14635427 | 4 | 47038000 | 4848218 | 5 |
| | 8 | 68767500 | 7969433 | 4 | 40606000 | 10750222 | 5 |
| | 18 | 38107500 | 9255540 | 4 | 96062000 | 25313313 | 5 |
| | 22 | 56917500 | 7445550 | 4 | 1.1E+08 | 60299107 | 5 |
| | 26 | 44572500 | 8581807 | 4 | 2.89E+08 | 1.86E+08 | 5 |
| | 29 | 60602500 | 18917399 | 4 | 5.74E+08 | 5.45E+08 | 5 |
| | 33 | 83882500 | 30944594 | 4 | 6.68E+08 | 6.13E+08 | 5 |
| | 36 | 75797500 | 29148427 | 4 | 1.42E+09 | 1.44E+09 | 5 |
| | 40 | 1.09E+08 | 61468496 | 4 | 1.7E+09 | 1.83E+09 | 5 |
| HLA-B | 1 | 75120000 | 13263039 | 4 | 95964000 | 11361716 | 5 |
| | 5 | 1.14E+08 | 2656478 | 4 | 54178000 | 11174948 | 5 |
| | 8 | 1.53E+08 | 30114147 | 4 | 86706000 | 15060945 | 5 |
| | 18 | 1.12E+08 | 19480373 | 4 | 70712000 | 17193704 | 5 |
| | 22 | 1.29E+08 | 23319638 | 4 | 1.31E+08 | 28386687 | 5 |
| | 26 | 2.18E+08 | 1.76E+08 | 4 | 1.02E+08 | 36123989 | 5 |
| | 29 | 3.19E+08 | 4.79E+08 | 4 | 1.79E+08 | 1.28E+08 | 5 |
| | 33 | 4.15E+08 | 6.04E+08 | 4 | 3.96E+08 | 4.33E+08 | 5 |
| | 36 | 5.16E+08 | 7.64E+08 | 4 | 6.5E+08 | 7.98E+08 | 5 |
| | 40 | 8.16E+08 | 1.3E+09 | 4 | 1.2E+09 | 1.63E+09 | 5 |
| HLA-AB | 1 | 54503800 | 31113691 | 4 | 40374000 | 5322889 | 5 |
| | 5 | 79315000 | 10423954 | 4 | 41694000 | 7467100 | 5 |
| | 8 | 79832500 | 12777999 | 4 | 34938000 | 8491715 | 5 |
| | 18 | 55077500 | 14565978 | 4 | 64404000 | 21336274 | 5 |
| | 22 | 46923250 | 33468493 | 4 | 43546000 | 16951128 | 5 |
| | 26 | 52755000 | 25167748 | 4 | 54498000 | 15582203 | 5 |
| | 29 | 66772500 | 37915812 | 4 | 51224000 | 14570855 | 5 |
| | 33 | 64415000 | 41401597 | 4 | 54594000 | 25814216 | 5 |
| | 36 | 52932500 | 30505738 | 4 | 52580000 | 16331823 | 5 |
| | 40 | 56737500 | 39401755 | 4 | 61682000 | 21887079 | 5 |
| NK only | 1 | | | | 1659800 | 144107.5 | 5 |
| | 5 | | | | 1252000 | 143766 | 5 |
| | 8 | | | | 1468667 | 21746.01 | 5 |
| | 18 | | | | 1321667 | 85253.87 | 5 |
| | 22 | | | | 1393333 | 114106.2 | 5 |
| | 26 | | | | 1329000 | 97857.04 | 5 |
| | 29 | | | | 1786000 | 227694.2 | 5 |
| | 33 | | | | 1290667 | 190931.6 | 5 |
| | 36 | | | | 1475000 | 197293.4 | 5 |
| | 40 | | | | 1505333 | 81667.35 | 5 |
| HBSS | 1 | 1335000 | 150939.7 | 5 | | | |
| | 5 | 1086560 | 154003 | 5 | | | |
| | 8 | 1116000 | 43446.52 | 5 | | | |
| | 18 | 1023540 | 60430.84 | 5 | | | |
| | 22 | 1214800 | 96373.03 | 5 | | | |
| | 26 | 1295000 | 109750.6 | 5 | | | |
| | 29 | 1145520 | 120455 | 5 | | | |
| | 33 | 1211400 | 60546.18 | 5 | | | |
| | 36 | 1319200 | 85889.23 | 5 | | | |
| | 40 | 1357000 | 112189.1 | 5 | | | |

### Example 16: Functional Analysis of T Cells with Double/Triple Knockout (KO) Edits and Expressing Anti-CD30 CAR-T

### 16.1. Engineering T Cells

Upon thaw (day 0), CD4+CD8+ T cells (Cellex) at a ratio of 1:1 were plated at a density of ˜1.2×10⁶ cells/mL in T cell activation media (TCAM) composed of CTS OpTmizer T cell Expansion SFM (Gibco, Cat. A3705001) containing CTS Supplement, 2.5% (v/v) of Human AB Serum (Valley Biomedical, HP1022HI), 1× Glutamax (Gibco, Cat. 35050-061), 10 mM HEPES buffer, 1% of Penicillin-Streptomycin, and 200 IU/mL of recombinant human interleukin-2 (Peprotech, Cat. 200-02), 5 ng/mL of recombinant human interleukin-7 (Peprotech, Cat. 200-07), and 5 ng/mL of recombinant human interleukin-15 (Peprotech, Cat. 200-15). Cells were incubated in a 37° C. incubator overnight.

24 hours post thaw (day 1), cells were counted and resuspended at 1×10⁶ cells/mL in TCAM. Cells were activated with TransAct™ (1:100 dilution, Miltenyi Biotec).

Different T cell groups were edited as described in Table 32. Briefly, LNP compositions containing 1) Nme2 BC22 mRNA and 2) sgRNA G034202 targeting HLA-A; or sgRNA G034201 targeting CIITA; or sgRNA G025420 targeting TRAC; or sgRNA G034209 targeting HLA-B. UGI mRNA LNP was separately formulated. See Example 1 describing LNP formulation.

TABLE 32
Editing scheme and viral transduction

| Condition | Day 0 | Day 1 | Day 3 | Day 5 |
|---|---|---|---|---|
| CD30 CAR | Thaw | TransAct | Nme2 BC22 HLA-A + | GREX 6 |
| Double KO | | | CIITA + Spy Cas9 | transfer |
| | | | TRAC + AAV | |
| CD30 CAR | Thaw | TransAct | Nme2 BC22 HLA-A + | GREX 6 |
| Triple KO | | | CIITA + HLA-B + | transfer |
| | | | Spy Cas9 TRAC + | |
| | | | AAV | |
| Untransduced/ | Thaw | TransAct | Spy Cas9 TRAC | GREX 6 |
| TCR KO only | | | | transfer |
| Unedited | Thaw | TransAct | | GREX 6 |
| | | | | transfer |

On day 3, cells were counted and resuspended in TCAM with cytokines as described above at 1×10⁶ cells/mL. A mixture of LNP, ApoE, AAV in TCAM was prepared at 2× concentration such that when equal volume of LNP mix was added to the T cells the concentration would be 1.5 μg/mL for each HLA-A, CIITA, or HLA-B sgRNA, with BC22 mRNA co-formulation LNPs, 0.2 μg/mL for UGI mRNA LNP, 1 μg/mL for TRAC sgRNA LNP, 10 μg/mL for ApoE, 1E5 GC/cell for AAV and the cells would be at final density 0.5×10⁶ cells/mL. Cells were incubated in a 37° C. incubator overnight.

24 hours post transfection (day 4), cells were counted and again brought to 0.5×10⁶ cells/mL density.

48 hours post transfection, (day 5) cells were cultured in 6-well GREX (Wilson Wolf, Cat. 80240M) in T cell expansion media (TCEM) composed of CTS OpTmizer T cell Expansion SFM (Gibco, Cat. A3705001) containing CTS Supplement, 5% (v/v) of Human AB Serum (Valley Biomedical, HP1022HI), 1× Glutamax (Gibco, Cat. 35050-061), 10 mM HEPES buffer, 1% of Penicillin-Streptomycin, and 200 IU/mL of recombinant human interleukin-2 (Peprotech, Cat. 200-02), 5 ng/mL of recombinant human interleukin-7 (Peprotech, Cat. 200-07), and 5 ng/mL of recombinant human interleukin-15 (Peprotech, Cat. 200-15). Cells were cultured till day 9 with regular media changes.

Additionally, T cells were also engineered to disrupt B2M and CIITA, to generate a double KO of these two genes.

Upon thaw (day 0), Donor DR26 T cells (Cellex) were plated at a density of ˜1.5×10⁶ cells/mL in T cell activation media (TCAM) composed of CTS OpTmizer T cell Expansion SFM (Gibco, Cat. A3705001) containing CTS Supplement, 2.5% (v/v) of Human AB Serum (Valley Biomedical, HP1022HI), 1× Glutamax (Gibco, Cat. 35050-061), 10 mM HEPES buffer, 1% of Penicillin-Streptomycin, and 200 IU/mL of recombinant human interleukin-2 (Peprotech, Cat. 200-02), 5 ng/mL of recombinant human interleukin-7 (Peprotech, Cat. 200-07), and 5 ng/mL of recombinant human interleukin-15 (Peprotech, Cat. 200-15). Cells were rested in a 37° C. incubator overnight.

Different T cell groups were edited as described in Table 33. Briefly, LNP compositions containing sgRNA G000529 (SEQ ID NO: 993) targeting B2M and sgRNA G013675 (SEQ ID NO: 3118) targeting CIITA.

TABLE 33
Order of editing and Harvest

| Condition | Day 0 | Day 1 | Day 2 | Day 3 | Day 8 |
|---|---|---|---|---|---|
| B2M + | Thaw | TransAct + | B2M | 24-well Grex | Harvest |
| CIITA | | CIITA | | transfer | |
| Unedited | Thaw | TransAct | | 24-well Grex | Harvest |
| | | | | transfer | |

24 hrs post thaw (day 1), cells were counted and resuspended in TCAM with cytokines as described above at 2×10⁶ cells/mL. Cells were activated with TransAct™ (1:100 dilution, Miltenyi Biotec). A mixture of LNP and ApoE3 in TCAM was prepared at 2× concentration such that when equal volume of LNP mix was added to T cells, the concentration would be 1.25 μg/mL for CIITA sgRNA formulation LNPs, and 2.5 μg/mL for ApoE and cell density would be 1×10⁶ cells/mL. Cells were incubated in a 37° C. incubator overnight.

On day 2, cells were counted and again brought to 1×10⁶ cells/mL density. A mixture of LNP and ApoE3 in TCAM was prepared at 10× concentration such that when equal volume of LNP mix was added to T cells resuspended at 1×10⁶ cells/mL, the concentration would be 2.5 μg/mL for B2M and 2.5 μg/mL for ApoE3. Cells were incubated in a 37° C. incubator overnight

24 hours later (on day 3) cells were cultured in 24-well GREX (Wilson Wolf, Cat. 80192M) in T cell expansion media (TCEM) composed of CTS OpTmizer T cell Expansion SFM (Gibco, Cat. A3705001) containing CTS Supplement, 5% (v/v) of Human AB Serum (Valley Biomedical, HP1022HI), 1× Glutamax (Gibco, Cat. 35050-061), 10 mM HEPES buffer, 1% of Penicillin-Streptomycin, and 200 IU/mL of recombinant human interleukin-2 (Peprotech, Cat. 200-02), 5 ng/mL of recombinant human interleukin-7 (Peprotech, Cat. 200-07), and 5 ng/mL of recombinant human interleukin-15 (Peprotech, Cat. 200-15). Cells were cultured until day 8 with regular media changes and frozen down on day 8.

### Example 16.2. Tumor Cell Killing Assay Using Engineered T Cells Expressing Anti-CD30 CAR-T Cells and with Double and Triple Knockout Edits

T cells were engineered with anti-CD30 CAR constructs and different edits as described in Example 16.1 and were tested for their cytotoxicity against CD30 expressing HH and MOLT-4 tumor cell lines. HH cells and MOLT-4 cells were thawed and maintained in culture for at least 7 days before setting up the killing assay. CD30 CAR-T cells and control T-cells were removed from liquid nitrogen, thawed, and rested overnight in pre-warmed T cell media. The killing assay was setup the following day by first seeding 20,000 cells/well of HH or MOLT-4 cells in 100 μL in a 96 well plate. CD30-CAR T-cells and control T-cells were counted, centrifuged, and resuspended in T-cell media without any cytokines. They were then serially diluted 3-fold starting with E:T ratio of 3.3 and then diluted up to 5 points. They were then added to target cells to their respective wells and kept in incubator at 37° C.

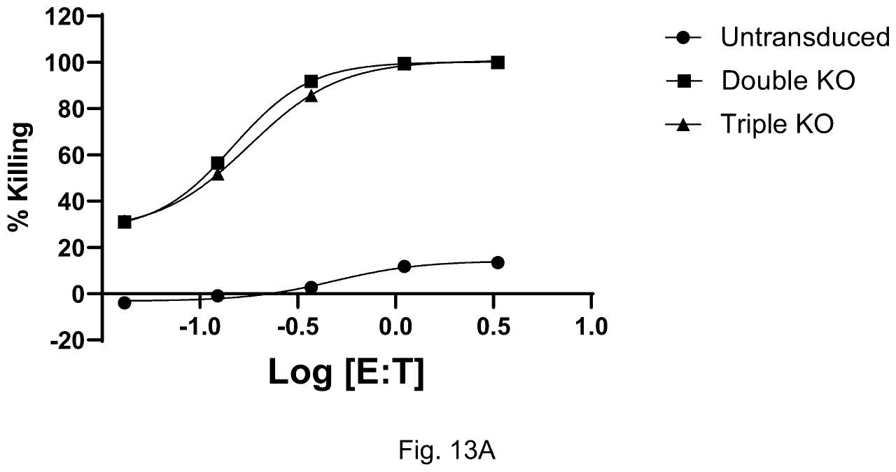

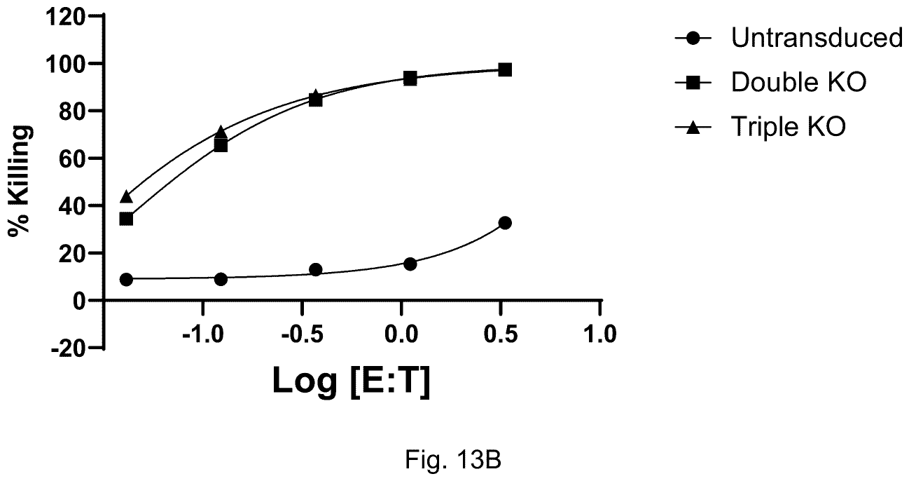

Bright-Glo™ Luciferase Assay System (Promega, Cat. E2620) was pre-thawed in dark at room temperature. The killing assay plate was taken out from the incubator. 50 μL of Bright-Glo™ Luciferase Assay System was added to each well and the plate was shaken briefly on a shaker and then incubated in dark at room temperature for 5 minutes. The plate was then read for luminescence with a CLARIOstar plate reader. The percentage killing was calculated from the luminescence with the average of T cell to tumor cell ratio 0 as 0% killing. The percent killing results are shown in for HH cells in Table 34 and FIG. 13A and the percent killing results for MOLT-4 cells are shown in Table 35 and FIG. 13B.

TABLE 34
percentage killing in HH cells for double and triple KO edits

| | Untransduced | | Double KO | | Triple KO | |
|---|---|---|---|---|---|---|
| | Mean % | SD % | Mean % | SD % | Mean % | SD % |
| E:T | Killing | Killing | Killing | Killing | Killing | Killing |
| 3.33 | 13.48 | 2.23 | 99.94 | 0.02 | 99.92 | 0.04 |
| 1.11 | 11.84 | 0.42 | 99.53 | 0.12 | 99.26 | 0.13 |
| 0.37 | 2.71 | 1.91 | 91.74 | 0.65 | 85.62 | 0.81 |
| 0.12 | −0.93 | 0.57 | 56.45 | 0.02 | 51.78 | 2.09 |
| 0.04 | −3.92 | 1.43 | 31.10 | 2.18 | 31.44 | 2.59 |

TABLE 35
percentage killing in MOLT-4 cells for double and triple KO edits

| | Untransduced | | Double KO | | Triple KO | |
|---|---|---|---|---|---|---|
| | Mean % | SD % | Mean % | SD % | Mean % | SD % |
| E:T | Killing | Killing | Killing | Killing | Killing | Killing |
| 3.33 | 32.73 | 0.48 | 97.37 | 0.17 | 97.29 | 0.64 |
| 1.11 | 15.28 | 1.08 | 94.08 | 0.01 | 93.36 | 0.99 |
| 0.37 | 12.97 | 0.28 | 84.65 | 1.60 | 86.61 | 0.90 |
| 0.12 | 8.90 | 0.81 | 65.51 | 2.59 | 71.31 | 1.11 |
| 0.04 | 8.79 | 0.72 | 34.37 | 0.95 | 44.03 | 2.53 |

### 16.3. MLR Assay

### Thawing & Resting Host PBMCs and Engineered Donor T Cells

Cryopreserved host PBMCs and engineered donor T cells as described in Table 36 were thawed at a cell concentration of 1.5×10⁶ cells/mL into T cell growth media (TCGM) composed of OpTmizer TCGM (Gibco, A1048501), Human Serum AB (GeminiBio, 100-512), HEPES 1M (Gibco, 15630-080), GlutaMAX Supplement (Gibco, 35050-061), and Penicillin-Streptomycin (Gibco, 15070-063) and further supplemented with 100 U/mL of recombinant human interleukin-2 (Peprotech, Cat. 200-02), 5 ng/mL IL-7 (Peprotech, Cat. 200-07), 5 ng/mL IL-15 (Peprotech, Cat. 200-15). Cells were rested in 37° C. incubator overnight.

TABLE 36
Donor T cells Editing Schematic and Host PBMCs

| Cell Type | | Donor | HLA-A | HLA-A | HLA-B | HLA-B | HLA-C | HLA-C |
|---|---|---|---|---|---|---|---|---|
| Donor T | WT (unedited) | Donor 1 | A*02:01 | A*02:01 | B*08:01 | B*08:01 | C*07:01 | C*07:01 |
| cells | | | | | | | | |
| | Triple KO | Donor 1 | A*02:01 | A*02:01 | B*08:01 | B*08:01 | C*07:01 | C*07:01 |
| | (HLA-A/HLA- | | | | | | | |
| | B/CIITA) | | | | | | | |
| Host | HLA-C Match | Host 1 | A*03:02 | A*03:02 | B*49:01 | B*49:01 | C*07:01 | C*07:01 |
| PBMCs | Host | | | | | | | |
| | Autologous | Auto Host | A*02:01 | A*02:01 | B*08:01 | B*08:01 | C*07:01 | C*07:01 |
| | | (Donor 1) | | | | | | |

Assay Setup with Engineered Donor T Cells and Host PBMCs

The following day, engineered donor T cells as described in Table 36 were irradiated at 5000 rad (Program C) and spun down, and each group was resuspended at 1×10⁶/mL in TCGM without cytokines. Host PBMCs will undergo CD56 depletion using CD56 microbeads from miltenyi as described in manufacturer's protocol. CD56 depleted were then counted using Nexcelom Celleca cell counter and desired number of cells were collected in a 15 mL conical tube and then spun down in a centrifuge at 500×G for 5 minutes followed by resuspending at 1×10⁶ cells/mL in phosphate buffered saline (Corning, Cat. No. 21-040-CV). A vial of Cell Trace Violet (Thermo Fisher, Cat. No. C34571) per donor was brought to room temperature and reconstituted using 20 μL DMSO to generate a stock of 5 mM CTV. A total of 10 million host PBMCs were transferred to a 50 mL conical tube and stained with 10 μL CTV at 5 μM concentration and incubated for 15 minutes in a 37° C. incubator. The labelled host cells were then spun down at 500×g for 5 minutes. CTV labelled host PBMCs were then resuspended at 1×10⁶ cells/mL in pre-warmed TCGM media composed of OpTimizer TCGM as described in Example 16.1. For the Donor+Host co-culture at 3:1 donor: host ratio, 50 μL of host PBMCs and 150 μL of donor T cells were added per each well in a sterile 96 well round bottom plate. The plate was transferred to 37° C. incubator and incubated for 6 days. On day 6 post co-culture, half the media (˜100 L) from each well was replaced with fresh media (TCGM without cytokines).

### Alloreactivity Readout by Flow Cytometry

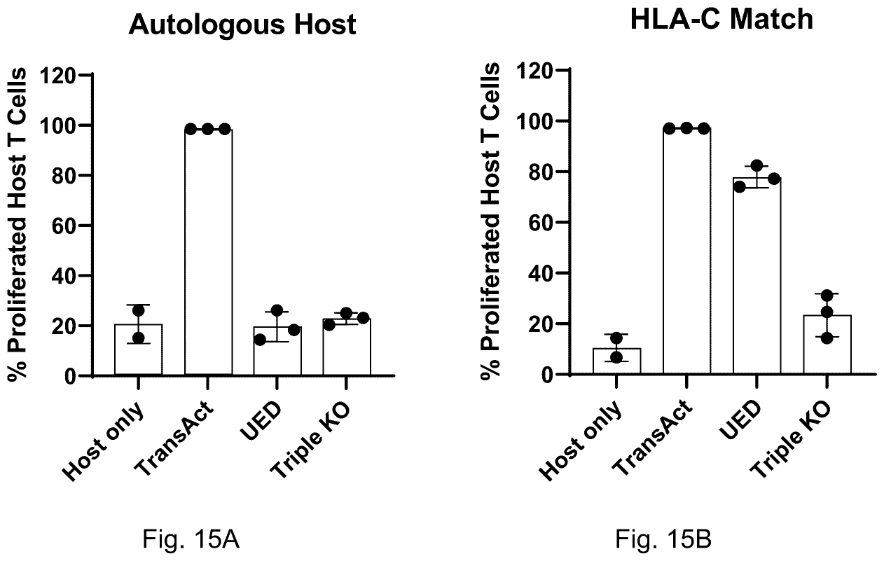

On day 8 post co-culture, the assay plate was stained and analyzed by flow cytometry. For the staining, the plate was spun at 600×g for 3 minutes, flicked to remove media, and 100 μL of a 1:100 v/v solution of Fc blocker (Biolegend, Cat #422302) in FACS buffer (Phosphate-buffered saline (Corning, Cat. 21-040-CV), Fetal Bovine Serum (Gibco, Cat. A3840201), UltraPure 0.5M EDTA (Invitrogen, Cat. 15575-020)) was added to each well. Cells were resuspended in the Fc blocker, and the plate was incubated at room temperature for 5 minutes. T cells were incubated with an antibody cocktail targeting the following molecules: CD3 (Biolegend, Cat. 317336), CD56 (Biolegend, Cat. 362546), CD4 (Biolegend, Cat. 300518), CD8 (Biolegend, Cat. 344742) and ViaKrome 808 Fixable Viability Dye (Beckman Coulter, Cat. C36628). Each antibody was prepared at a 1:100 v/v dilution, and 100 μL of this antibody mixture was added to each sample well. The plate was protected from light by covering with an aluminum foil and incubated at 2-8° C. for 20-30 minutes. After staining, the plate was spun at 600×g for 3 minutes, flicked to remove media and washed with 200 μL of FACS buffer. The plate was washed again, and the cell pellets were resuspended in 60 μL of FACS buffer. To each well, 10 μL of Count Bright Absolute Counting Beads was added and mixed well. The plate was run and recorded on fast mode with 70 μL total volume as a stopping rule on Cytoflex flow cytometer. FIGS. 15A and 15B show the percentage of host T cell proliferation as a result of donor T cell treatment. FIG. 15A shows that, in an autologous context, host T cells co-cultured with unedited T cells (UED) or edited T cells (Triple KO) show similar proliferation as host T cells alone. FIG. 15B shows host T cells co-cultured with unedited T cells (UED) show higher proliferation due to mismatch of HLA-A and B alleles (while C is matched) as compared to host only control. Additionally, host T cells co-cultured with unedited T cells (UED) show higher proliferation due to mismatch of HLA-A and B alleles (while C is matched), as compared to triple KO engineered cells, where HLA-A and HLA-B are disrupted, and HLA-C is matched.

### In Vitro NK Cell Killing Assays

CD30-CAR T cells edited in various combinations to disrupt CIITA, HLA-A, HLA-B, and/or B2M as described in Example 16.1, were tested for their ability to resist natural killer (NK) cell mediated killing.

### Thawing & Resting Host PBMCs and Engineered Donor T Cells

Cryopreserved NK cells and donor T cells as described in Table 36 were thawed at a cell concentration of 1.5×10⁶ cells/ml into T cell growth media (TCGM) composed of OpTmizer TCGM as described in Example 16.3 and further supplemented with 100 U/mL of recombinant human interleukin-2 (Peprotech, Cat. 200-02), 5 ng/mL IL-7 (Peprotech, Cat. 200-07), 5 ng/mL IL-15 (Peprotech, Cat. 200-15) for T-cells and 500 U/mL of IL-2 only for NK cells. Cells were rested in 37° C. incubator overnight.

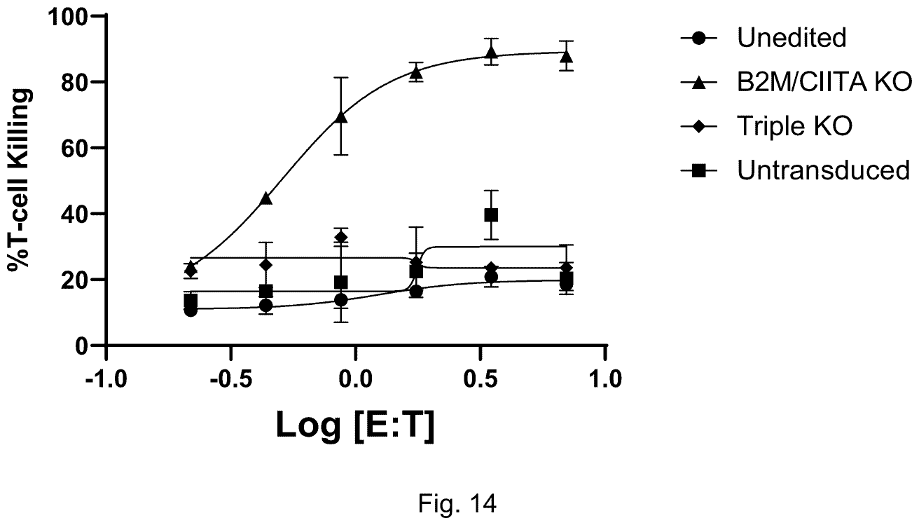

Flow cytometry was performed as in Example 11.2. Table 37 shows the genotypes of the donor T cells. Table 38 and FIG. 14 show the mean percentage of T cell lysis following NK cell challenge.

TABLE 37
Donor T cells and Host NK cells

| Cell Type | | Donor ID | HLA-A | HLA-A | HLA-B | HLA-B | HLA-C | HLA-C |
|---|---|---|---|---|---|---|---|---|
| | WT | Donor 1 | A*02:01 | A*02:01 | B*08:01 | B*08:01 | C*07:01 | C*07:01 |
| Donor T | B2M + CIITA | Donor 1 | A*02:01 | A*02:01 | B*08:01 | B*08:01 | C*07:01 | C*07:01 |
| cells | Triple KO | Donor 1 | A*02:01 | A*02:01 | B*08:01 | B*08:01 | C*07:01 | C*07:01 |
| | (HLA-A/HLA- | | | | | | | |
| | B/CIITA) | | | | | | | |

TABLE 38
T cell lysis following NK cell challenge

| | | | | | B2M/ | | | |
|---|---|---|---|---|---|---|---|---|
| | Unedited | | Untransduced | | CIITA KO | | Triple KO | |
| | Mean | | Mean | | Mean | | Mean | |
| | % | SD % | % | SD % | % | SD % | % | SD % |
| E:T | Killing | Killing | Killing | Killing | Killing | Killing | Killing | Killing |
| 7.00 | 18.65 | 1.91 | 20.35 | 4.88 | 87.98 | 4.50 | 23.60 | 6.93 |
| 3.50 | 20.90 | 3.11 | 39.65 | 7.42 | 89.18 | 4.07 | 23.55 | 1.48 |
| 1.75 | 16.45 | 0.21 | 22.45 | 5.59 | 83.00 | 2.97 | 25.25 | 10.68 |
| 0.88 | 13.85 | 2.62 | 19.20 | 12.16 | 69.65 | 11.81 | 32.85 | 2.76 |
| 0.44 | 12.20 | 2.69 | 16.50 | 1.70 | 44.90 | 0.42 | 24.45 | 6.86 |
| 0.22 | 10.70 | 0.28 | 13.70 | 2.69 | 24.00 | 0.00 | 22.60 | 2.26 |

### Example 17: Double and Triple Knockout Edits in Differentiated iPSC Cells

iPSCs were reprogrammed from human PBMCs at CORM (Centre for Commercialization of Regenerative Medicine). After multiple clone characterization assays, including pluripotency marker expression and karyotyping, a single iPSC clone was selected for CRISPR/Cas9 mediated gene-editing. The selected iPSC cells were edited using HLA-A guide G018995, HLA-B guide G022020, and CIITA guide G013675, to generate double knockout (HLA-A and CIITA DKO) or triple knockout (HLA-A, HLA-B, and CIITA TKO) samples.

As shown in Table 39 below, ddPCR data showed high editing efficiency in all three targets in bulk iPSC cells.

TABLE 39
Editing Efficiencies for HLA-A, CIITA, and HLA-B

| | HLA-A Editing | CIITA Editing | HLA-B Editing |
|---|---|---|---|
| | (%) | (%) | (%) |
| WT unedited | −5 | 0 | −8 |
| DKO | 87 | 84 | N/A |
| TKO | 87 | 84 | 79 |

After single cell plating, clonal expansion, and clone characterization, a DKO or TKO iPSC clone was further selected to differentiate into cardiomyocytes or pancreatic progenitors. Differentiation was also carried out at CCRM.

Differentiated cardiomyocytes and pancreatic progenitors were verified at CCRM. Briefly, the differentiated cardiomyocytes were assayed for a cardiac marker. More than 80% of the differentiated iPSCs from different groups (wild type, DKO, and TKO) were positive for cardiac Troponin (cTNT), a cardiac marker. Similarly, after differentiation, more than 80% wild type or TKO cells were observed to express pancreatic and duodenal homeobox 1 (PDX1) and NK6 homeobox 1 (NKX6.1), two key transcription factors that drive the development of beta cells. These data suggest DKO or TKO iPSCs have comparable differentiation capability as wild type iPSCs.

1. An engineered human cell, which has reduced or eliminated surface expression of:
I) HLA-A and HLA-B protein relative to an unmodified cell, comprising a genetic modification in the HLA-A gene and a genetic modification in the HLA-B gene, wherein the cell is homozygous for HLA-C;
II) HLA-A and HLA-B protein relative to an unmodified cell, comprising a genetic modification in the HLA-A gene and a genetic modification in the HLA-B gene, wherein
(i) the genetic modification in the HLA-A gene comprises at least one nucleotide within the genomic coordinates chosen from: (a) chr6:29942854-chr6:29942913 and chr6:29943518-chr6:29943619, and (b) chr6:29942540-29945459, and
(ii) the genetic modification in the HLA-B gene comprises at least one nucleotide within the genomic coordinates chosen from: (a) chr6:31354480-31357174 or (b) chr6:31354497-31357157;
III) HLA-B protein relative to an unmodified cell, comprising a genetic modification in the HLA-B gene, wherein the cell is homozygous for HLA-A and homozygous for HLA-C: or
IV) HLA-B protein relative to an unmodified cell, comprising a genetic modification in the HLA-B gene, wherein the genetic modification comprises at least one nucleotide within the genomic coordinates chosen from: (a) chr6:31354480-31357174 and (b) chr6:31354497-31357157.

2. (canceled)

3. (canceled)

4. (canceled)

5. The engineered human cell of claim 1, wherein the cell has reduced or eliminated expression of:
a) at least one HLA-B allele selected from HLA-B7, HLA-B8, HLA-B35, HLA-B40, HLA-B44, HLA-B15, HLA-B14, HLA-B18 and HLA-B51; and/or
b) at least one HLA-A allele selected from: HLA-A1, HLA-A2, HLA-A3, HLA-A11, HLA-A29, HLA-A26, HLA-A33, and HLA-A24.

6. (canceled)

7. The engineered human cell of claim 1, wherein the genetic modification in the HLA-B gene comprises at least one nucleotide within the genomic coordinates chosen from: (a) chr6:31355182-31355596; (b) chr6:31355203-31356461;
(c) chr6:31355182-31355202 chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; chr6:31355414-31355434; and chr6:31355409-31355429;
(d) chr6:31355222-31355246; chr6:31356777-31356801: chr6:31355492-31355516; chr6: 31355379-31355403; chr6:31355491-31355515; chr6:31355361-31355385; chr6:31355356-31355380; chr6:31355460-31355484; chr6:31357078-31357102; chr6:31355417-31355441; chr6:31355366-31355390; chr6:31355415-31355439; chr6:31355378-31355402; chr6:31355166-31355190; chr6:31355401-31355425; ch6:31355469-31355493; chr6:31356262-31356286: chr6:31355419-31355443: chr6:31355390-31355414 chr6:31355369-31355393; chr6:31355221-31355245; chr6:31355205-31355229; chr6:31355446-31355470; chr6:31356425-31356449; chr6:31355441-31355465; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; and chr6:31356767-31356791;
(e) chr6:31355348-31355368, chr6:31355347-31355367, chr6:31355349-31355369, chr6:31355192-31355212, chr6:31355340-31355360, and chr6:31355409-31355429; and
(f) chr6:31355222-31355246; chr6:31355221-31355245: chr6:31355205-31355229; chr6:31355446-31355470; chr6:31356425-31356449; and chr6:31355441-31355465.

8. (canceled)

9. (canceled)

10. (canceled)

11. The engineered human cell of claim 1, wherein the genetic modification in the HLA-A gene comprises at least one nucleotide within the genomic coordinates chosen from:
a) chr6:29942891-29942915: chr6:29942609-29942633: chr6:29942864-29942884;
chr6:29944266-29944290; chr6:29942889-29942913; chr6:29944471-29944495; and
chr6:29944470-29944494;
b) chr6:29942891-29942915;
c) chr6:29942864-29942884; chr6:29942868-29942888 chr6:29942876-29942896 chr6:29942877-29942897; and chr6:29942883-29942903; and
d) chr6:29942609-29942633.

12. (canceled)

13. (canceled)

14. (canceled)

15. (canceled)

16. (canceled)

17. (canceled)

18. (canceled)

19. The engineered human cell of claim 1, wherein the HLA-B expression is reduced or eliminated by:
a) a gene editing system that binds to an HLA-B genomic target sequence comprising at least 5 or at least 10 contiguous nucleotides within the genomic coordinates chosen from: chr6:31355348-31355368; or chr6:31355347-31355367; chr6:31355182-31355202; chr6:31355348-31355368; chr6:31355180-31355200; chr6:31355145-31355165; chr6:31355349-31355369; chr6:31355157-31355177; chr6:31356381-31356401; chr6:31356380-31356400; chr6:31355204-31355224; chr6:31355205-31355225; chr6:31355185-31355205; chr6:31355191-31355211; chr6:31355192-31355212; chr6:31355190-31355210; chr6:31355193-31355213; chr6:31355198-31355218; chr6:31355320-31355340; chr6:31355319-31355339; chr6:31355178-31355198; chr6:31355347-31355367; chr6:31355432-31355452; chr6:31355340-31355360; chr6:31355576-31355596; chr6:31355410-31355430; chr6:31355419-31355439; and chr6:31355414-31355434; chr6:31355409-31355429; or
b) a gene editing system that binds to an HLA-B genomic target sequence comprising at least 5 or at least 10 contiguous nucleotides within the genomic coordinates chosen from: chr6:31355222-31355246: chr6:31355221-31355245: chr6:31355205-31355229; chr6:31355446-31355470: chr6:31356425-31356449: chr6:31355441-31355465; chr6:31356777-31356801: chr6:31355492-31355516: chr6: 31355379-31355403; chr6:31355491-31355515: chr6:31355361-31355385: chr6:31355356-31355380; chr6:31355460-31355484: chr6:31357078-31357102: chr6:31355417-31355441; chr6:31355366-31355390: chr6:31355415-31355439: chr6:31355378-31355402; chr6:31355166-31355190: chr6:31355401-31355425: ch6:31355469-31355493; chr6:31356262-31356286; chr6:31355419-31355443; chr6:31355390-31355414; chr6:31355369-31355393; chr6:31355203-31355227; chr6:31356437-31356461; chr6:31356426-31356450; chr6:31356763-31356787; chr6:31356764-31356788; chr6:31356762-31356786; chr6:31355204-31355228; chr6:31356436-31356460; and chr6:31356767-31356791.

20. (canceled)

21. The engineered human cell of claim 1, wherein HLA-A expression is reduced or eliminated by a gene editing system that binds to an HLA-A genomic target sequence comprising at least 5 or at least 10 contiguous nucleotides within the genomic coordinates chosen from: (a) chr6:29942891-29942915; chr6:29942609-29942633; chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; chr6:29944026-29944046; chr6:29934330-29934350, chr6:29943115-29943135, chr6:29943135-29943155, chr6:29943140-29943160, chr6:29943590-29943610, chr6:29943824-29943844, chr6:29943858-29943878, chr6:29944478-29944498, and chr6:29944850-29944870; and (b) chr6:29942891-29942915 and chr6:29942609-29942633.

22. (canceled)

23. (canceled)

24. The engineered human cell of claim 1, wherein the cell is homozygous for HLA-C.

25. The engineered human cell of claim 1, wherein the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*08:01; HLA-C*03:02; HLA-C*06:02; HLA-C*16:01; HLA-C*12:03; HLA-C*04:01; HLA-C*15:02; HLA-C*07:01; HLA-C*03:04; HLA-C*12:03; HLA-C*02:10; HLA-C*05:01; HLA-C*12:02; HLA-C*14:02; HLA-C*06:02; HLA-C*04:01; HLA-C*03:03; HLA-C*07:04; HLA-C*07:04; HLA-C*04:01; HLA-C*17:01; HLA-C*01:02; and HLA-C*02:02.

26. The engineered human cell of claim 1, wherein the engineered cell is homozygous for HLA-A, the HLA-A allele is selected from any one of the following HLA-A alleles: HLA-A*02:01; HLA-A*01:01; HLA-A*03:01; HLA-A*11:01; HLA-A*26:01; HLA-A*68:01; HLA-A*29:02; HLA-A*31:01; HLA-A*32:01; HLA-A*30:02; HLA-A*25:01; HLA-A*33:01; HLA-A*02:02; HLA-A*74:01; HLA-A*02:02; HLA-A*29:01; HLA-A*02:03; HLA-A*02:05; HLA-A*24:07; HLA-A*11:02; HLA-A*36:01; HLA-A*02:22; HLA-A*34:02; HLA-A*01:03; HLA-A*24:02; HLA-A*02:07; HLA-A*23:01; HLA-A*30:01; HLA-A*33:03; HLA-A*02:06; HLA-A*34:02; and HLA-A*68:02.

27. The engineered human cell of claim 1, wherein the engineered cell is homozygous for HLA-A and wherein the engineered cell is homozygous for HLA-C, wherein the HLA-A allele is selected from any one of the following HLA-A alleles: HLA-A*02:01; HLA-A*01:01; HLA-A*03:01; HLA-A*11:01; HLA-A*26:01; HLA-A*68:01; HLA-A*29:02; HLA-A*31:01; HLA-A*32:01; HLA-A*30:02; HLA-A*25:01; HLA-A*33:01; HLA-A*02:02; HLA-A*74:01; HLA-A*02:02; HLA-A*29:01; HLA-A*02:03; HLA-A*02:05; HLA-A*24:07; HLA-A*11:02; HLA-A*36:01; HLA-A*02:22; HLA-A*34:02; HLA-A*01:03; HLA-A*24:02; HLA-A*02:07; HLA-A*23:01; HLA-A*30:01; HLA-A*33:03; HLA-A*02:06; HLA-A*34:02; and HLA-A*68:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*08:01; HLA-C*03:02; HLA-C*16:01; HLA-C*15:02; HLA-C*03:04; HLA-C*12:03; HLA-C*02:10; HLA-C*05:01; HLA-C*12:02; HLA-C*14:02; HLA-C*04:01; HLA-C*03:03; HLA-C*07:04; HLA-C*17:01; HLA-C*01:02; and HLA-C*02:02.

28. The engineered human cell of claim 1, wherein the cell has:
a) reduced or eliminated surface expression of MHC class II protein,
b) a genetic modification of a gene selected from CIITA, HLA-DR, HLA-DQ, HLA-DP, RFX5, RFXB/ANK, RFXAP, CREB, NF-YA, NF-YB, and NF-YC;
c) a genetic modification in the CIITA gene;
d) reduced or eliminated surface expression of TRAC protein; and/or
e) reduced or eliminated surface expression of TRBC protein.

29. (canceled)

30. (canceled)

31. (canceled)

32. (canceled)

33. The engineered human cell of claim 1, wherein the genetic modification comprises:
a) at least 2, at least 3, at least 4, at least 5, at least 6, at least 7, at least 8, at least 9, or at least 10 contiguous nucleotides within the genomic coordinates;
b) an indel; and/or
c) at least one C to T substitution or at least one A to G substitution within the genomic coordinates.

34. (canceled)

35. (canceled)

36. A pharmaceutical composition comprising the engineered human cell of claim 1.

37. A population of cells comprising the engineered human cell of claim 1.

38. A pharmaceutical composition comprising the population of cells of claim 37.

39. The population of claim 37, wherein
a) at least 65%, at least 70%, at least 80%, at least 90%, at least 91%, at least 92%, at least 93%, at least 94%, at least 95%, at least 96%, at least 97%, at least 98%, or at least 99% of the population of cells is HLA-A negative or HLA-B negative as measured by flow cytometry;
b) at least 65%, at least 70%, at least 80%, at least 85%, at least 90%, at least 95%, at least 96%, at least 97%, at least 98%, or at least 99% of the population of cells comprises the genetic modification in the HLA-A gene or the genetic modification in the HLA-B gene, as measured by next-generation sequencing (NGS);
c) at least 94%, at least 95%, at least 96%, at least 97%, at least 98%, or at least 99% of the population of cells is CIITA negative as measured by flow cytometry; and/or
d) at least 95%, at least 97%, at least 98%, at least 99%, or at least 99.5% of the population of cells is endogenous TCR protein negative as measured by flow cytometry.

40. (canceled)

41. (canceled)

42. (canceled)

43. (canceled)

44. (canceled)

45. A method of treating a disease or disorder comprising administering the engineered human cell of claim 1 to a subject in need thereof, optionally wherein the disease or disorder is a cancer, an infectious disease, or an autoimmune disease.

46. A composition, comprising:
a) an HLA-B guide RNA; or
b) an HLA-B guide RNA and an HLA-A guide RNA,
wherein the HLA-B guide RNA comprises:
i. a guide sequence selected from SEQ ID NOs: 165, 166, 177, 13, 74, 1-12, 14-73, 75-91, 101-164, 167-176, and 178-185;
ii. at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185;
iii. a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185;
iv. a guide sequence that binds a target site comprising a genomic region listed in Table 2 or 3; or
v. a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3; and
wherein the HLA-A guide RNA, if present, comprises:
i. a guide sequence selected from SEQ ID NOs: 576, 571, 301-570, 572-575, and 577-590; or
ii. at least 17, 18, 19, or 20 contiguous nucleotides of a sequence selected from SEQ ID NOs: 301-428 and 463-511: or at least 17, 18, 19, 20, 21, 22, 23, or 24 contiguous nucleotides of a sequence selected from SEQ ID NOs: 429-462 and 512-590; or
iii. a guide sequence at least 95%, 90%, or 85% identical to a sequence selected from SEQ ID NOs: 301-428 and 463-511: or a guide sequence at least 95%, 90%, 85%, 80%, 75%, or 70% identical to a sequence selected from SEQ ID NOs: 512-590; or
iv. a guide sequence that binds a target site comprising a genomic region listed in Tables 4-7; or
v. a guide sequence that is complementary to at least 17, 18, 19, 20, 21, 22, 23, or 24 contiguous nucleotides of a genomic region listed in Tables 4-7.

47. (canceled)

48. A method of making an engineered human cell, wherein the engineered human cell has reduced or eliminated surface expression of:
I) HLA-B protein relative to an unmodified cell, the method comprising: contacting a cell with a composition comprising an HLA-B guide RNA; or
II) HLA-A and HLA-B protein relative to an unmodified cell, the method comprising: (a) contacting a cell with a first composition comprising an HLA-B guide RNA; and (b) contacting the cell with a second composition comprising an HLA-A guide RNA,
wherein the HLA-B guide RNA comprises:
i. a guide sequence selected from SEQ ID NOs: 165, 166, 177, 13, 74, 1-12, 14-73, 75-91, 101-164, 167-176, and 178-185;
ii. at least 17, 18, 19, or 20 contiguous nucleotides of a sequence selected from SEQ ID NOs: 1-91; or at least 17, 18, 19, 20, 21, 22, 23, or 24 contiguous nucleotides of a sequence selected from SEQ ID NOs: 101-185;
iii. a guide sequence at least 95%, 90%, or 85% identical to a sequence selected from SEQ ID NOs: 1-91; or a guide sequence at least 95%, 90%, 85%, 80%, 75%, or 70% identical to a sequence selected from SEQ ID NOs: 101-185;
iv. a guide sequence that binds a target site comprising a genomic region listed in Table 2 or 3; or
v. a guide sequence that is complementary to at least 17, 18, 19, or 20 contiguous nucleotides of a genomic region listed in Table 2 or a guide sequence that is complementary to at least 17, 18, 19, 20, 21, 22, 23, or 24 contiguous nucleotides of a genomic region listed in Table 3;
wherein the HLA-A guide RNA, if present, comprises:
i. a guide sequence selected from SEQ ID NOs: 576, 571, 301-570, 572-575, and 577-590; or
ii. at least 17, 18, 19, or 20 contiguous nucleotides of a sequence selected from SEQ ID NOs: 301-428 and 463-511: or at least 17, 18, 19, 20, 21, 22, 23, or 24 contiguous nucleotides of a sequence selected from SEQ ID NOs: 429-462 and 512-590; or
iii. a guide sequence at least 95%, 90%, or 85% identical to a sequence selected from SEQ ID NOs: 301-428 and 463-511: or a guide sequence at least 95%, 90%, 85%, 80%, 75%, or 70% identical to a sequence selected from SEQ ID NOs: 512-590; or
iv. a guide sequence that binds a target site comprising a genomic region listed in Tables 4-7; or
v. a guide sequence that is complementary to at least 17, 18, 19, 20, 21, 22, 23, or 24 contiguous nucleotides of a genomic region listed in Tables 4-7.

49. (canceled)

50. (canceled)

51. (canceled)

52. (canceled)

53. (canceled)

54. The engineered human cell of claim 1, further comprising an RNA-guided DNA-binding agent or nucleic acid encoding the RNA-guided DNA binding agent, wherein the RNA-guided DNA-binding agent is NmeCas9, and the HLA-B guide RNA comprises: (i) a guide sequence selected from SEQ ID NOs: 165, 166, 163, 164, 169, and 177; or (ii) a guide sequence that is at least 17, 18, 19, 20, 21, 22, 23, or 24 contiguous nucleotides of a sequence selected from SEQ ID NOs: 165, 166, 163, 164, and 177; or (iii) a guide sequence at least 95%, 90%, 85%, 80%, 75%, or 70% identical to a sequence selected from SEQ ID NOs: 165, 166, 163, 164, and 177.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. The engineered human cell of claim 1, wherein the cell is an allogeneic cell and/or a stem cell.

67. (canceled)

68. The engineered human cell of claim 1, further comprising:
a) an exogenous nucleic acid encoding a polypeptide, wherein the polypeptide is an antibody or antibody fragment;
b) an exogenous nucleic acid encoding a polypeptide that is secreted by the cell, wherein the secreted polypeptide is an enzyme;
c) an exogenous nucleic acid encoding a polypeptide that is secreted by the cell, wherein the secreted polypeptide is a cytokine;
d) an exogenous nucleic acid encoding a targeting receptor, wherein the targeting receptor is a T cell receptor (TCR);
e) an exogenous nucleic acid encoding a targeting receptor, wherein the targeting receptor is a genetically modified TCR;
f) an exogenous nucleic acid encoding a targeting receptor, wherein the targeting receptor is a WT1 TCR;
g) an exogenous nucleic acid encoding a targeting receptor, wherein the targeting receptor is a CAR;
h) an exogenous nucleic acid encoding a targeting receptor, wherein the targeting receptor is a universal CAR; or
i) an exogenous nucleic acid encoding a targeting receptor, wherein the targeting receptor is an anti-CD30 CAR.

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. The engineered human cell of claim 68, wherein the exogenous nucleic acid is provided to the cell in a vector, optionally wherein the vector is a viral vector.

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. A cell bank comprising: (a) the engineered human cell of claim 1; and (b) a catalogue comprising information documenting the HLA-C alleles of the cell in the cell bank.

112. A method of administering an engineered human cell to a recipient subject in need thereof, the method comprising: (a) determining the HLA-C alleles of the recipient subject; (b) selecting the engineered human cell of claim 1, wherein the engineered human cell is homozygous for one of the HLA-C alleles of the recipient subject; and (c) administering the selected engineered human cell to the recipient subject.
